(19) United States
(12) Patent Application Publication
(10) Pub. No.: US 2021/0275518 A1
(43) Pub. Date: Sep. 9, 2021

(54) METHODS OF USING REBASTINIB IN THE TREATMENT OF DISORDERS

(71) Applicant: Deciphera Pharmaceuticals, LLC, Waltham, MA (US)

(72) Inventors: Rodrigo Ruiz Soto, Waltham, MA (US); Keisuke Kuida, Waltham, MA (US); Haroun Achour, Waltham, MA (US)

(21) Appl. No.: 17/193,707

(22) Filed: Mar. 5, 2021

Related U.S. Application Data
(60) Provisional application No. 63/121,097, filed on Dec. 3, 2020.
(60) Provisional application No. 63/079,356, filed on Sep. 16, 2020.
(60) Provisional application No. 63/023,925, filed on May 13, 2020.
(60) Provisional application No. 62/986,222, filed on Mar. 6, 2020.

(51) Int. Cl. A61K 31/4709; A61K 31/337; A61K 31/475; A61K 31/436; A61K 31/404; A61K 31/704; A61K 31/44; A61K 31/513; A61K 31/7068; A61K 31/519; A61K 31/495; A61K 33/243; A61K 31/282; A61K 31/675; A61K 31/427; A61K 31/517; A61K 31/496; A61K 31/4545; A61K 31/506; A61K 31/437; A61K 31/454; A61K 31/502; A61K 31/55; A61P 35/00

(52) U.S. Cl. CPC A61K 31/4709; A61P 35/00; A61K 31/475; A61K 31/436; A61K 31/404; A61K 31/704; A61K 31/44; A61K 31/513; A61K 31/7068; A61K 31/519; A61K 31/495; A61K 33/243; A61K 31/282; A61K 31/675; A61K 31/427; A61K 31/517; A61K 31/496; A61K 31/4545; A61K 31/506; A61K 31/437; A61K 31/454; A61K 31/502; A61K 31/55; A61K 31/337

(57) ABSTRACT

Described herein are methods of treating various disorders in patients in need thereof, comprising administering to the patient the compound of Formula (I)

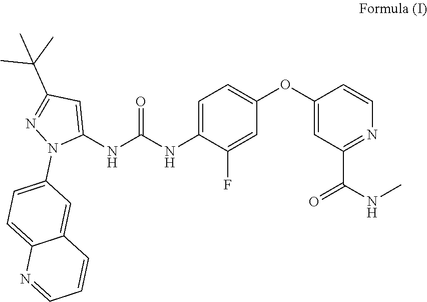 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof. Exemplary disorders that can be treated by the methods described herein include gynecologic carcinosarcomas, endometrial adenocarcinomas, mesotheliomas, ovarian cancers, pancreatic ductal adenocarcinomas, and lung cancers.

### CROSS REFERENCES TO RELATED APPLICATIONS

The application claims priority to U.S. Ser. No. 62/986,222 filed Mar. 6, 2020, U.S. Ser. No. 63/023,925 filed May 13, 2020, U.S. Ser. No. 63/079,356 filed Sep. 16, 2020, and U.S. Ser. No. 63/121,097 filed Dec. 3, 2020, the contents of each of which are incorporated herein by reference.

### BACKGROUND

The angiopoietin (ANG)/tunica internal endothelial cell kinase 2 (TIE2) kinase signaling pathway is a pivotal angiogenic signaling axis in endothelial cells. TIE2 is also expressed on a subset of macrophages (i.e., TIE2-expressing macrophages [TEMs]) which not only promote cancer growth, cancer cell survival, and motility, but also can limit the efficacy of the tumor response to chemotherapy or radiotherapy. In particular, TEMs are known to be proangiogenic, prometastatic, and immunosuppressive in the tumor microenvironment. Since TIE2 expression is restricted to endothelial cells and highly protumoral TEMs, TIE2 is an attractive target for interrupting tumor cell/microenvironment interactions. Regarding its role in tumor angiogenesis, TIE2 signaling is necessary for upregulation and function of protumoral TEMs that mediate angiogenesis in breast cancer and pancreatic islet tumors syngeneic mouse models, and adaptive or evasive revascularization in pancreatic neuroendocrine cancer treated with anti-vascular endothelial growth factor (VEGF) therapy. Chemotherapeutic regimens have been shown to recruit TIE2-expressing monocytes to tumor sites, promoting tumor angiogenesis and tumor invasion and metastasis. Proangiogenic TEMs have also been shown to limit therapeutic effectiveness of vascular disrupting agents.

It has been demonstrated that TEMs mediate invasion and metastasis in the polyoma middle T antigen syngeneic breast cancer model (PyMT), and that TIE2 expression correlates with poor overall survival (OS) and a high risk of metastasis in breast cancer patients. Perivascular TEMs assemble into special structures with endothelial and tumor cells termed tumor microenvironment of metastasis (TMEM) to promote tumor cell intravasation into the blood stream, leading to circulating tumor cells (CTCs) and systemic dissemination and metastasis. Moreover, chemotherapy increases the density and activity of TMEM sites and promotes distant metastasis in the PyMT and patient-derived xenografts. Inhibition of TMEM function may thus improve clinical benefits of chemotherapy in the neoadjuvant setting or in metastatic disease.

It has also been demonstrated that TIE2 expression can be upregulated in ovarian cancer cells and mesothelioma cells, conferring a tumor cell autonomous expression in addition to TIE2 expression in the tumor microenvironment. It has also been reported that TIE2 signaling in pancreatic ductal adenocarcinoma or lung cancer contributes to a protumoral tumor microenvironment enabling cancer progression and metastasis.

There is accordingly a need to develop new therapies to treat patients suffering from various cancers and related disorders using TIE2 kinase inhibitors.

### SUMMARY

Provided herein, in one embodiment, are methods of treating various disorders, such as a gynecologic carcinosarcoma, endometrial adenocarcinoma, mesothelioma, ovarian cancer, pancreatic ductal adenocarcinoma, or lung cancer, in a patient in need thereof, comprising administering to the patient an effective amount of the compound of Formula (I):

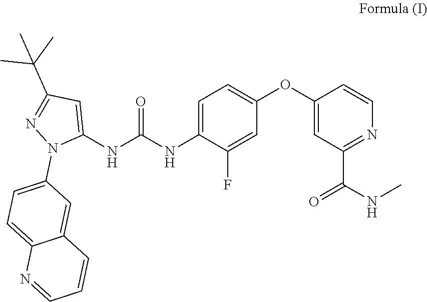 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In one embodiment, provided herein is a method of treating a gynecologic carcinosarcoma in a patient that has previously received at least one line of treatment for the gynecologic carcinosarcoma, and is in need thereof, comprising administering to the patient an effective amount of the compound of Formula (I):

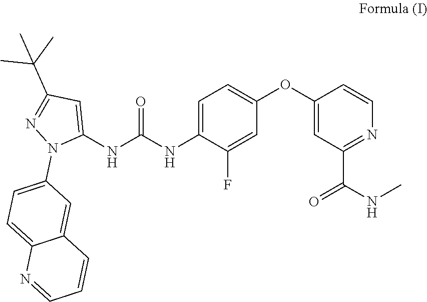 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In another embodiment, described herein is a method of treating a gynecologic carcinosarcoma in a patient that has previously received at least one line of treatment for the gynecologic carcinosarcoma, and is in need thereof, comprising administering to the patient an effective amount of: a) the compound of Formula (I)

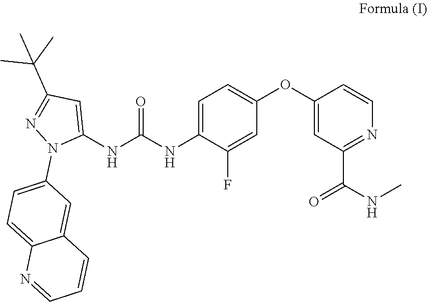 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof; and b) paclitaxel.

In another embodiment, also provided herein is a method of treating endometrial adenocarcinoma in a patient in need thereof, comprising administering to the patient an effective amount of the compound of Formula (I)

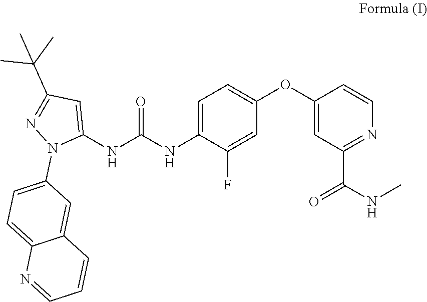 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In another embodiment, provided herein is a method for achieving at least a partial response in a patient having endometrial adenocarcinoma, comprising administering to the patient an effective amount of the compound of Formula (I)

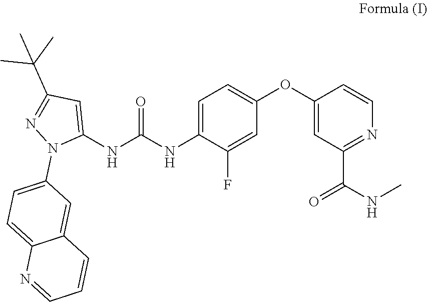 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In another embodiment, provided herein is a method for achieving a stable disease condition in a patient having endometrial adenocarcinoma, comprising administering to the patient an effective amount of the compound of Formula (I)

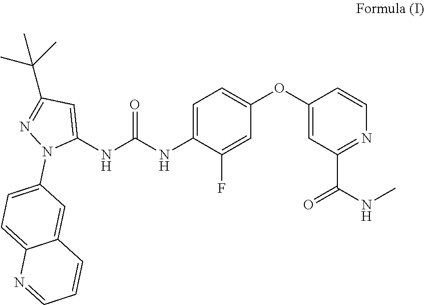 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In one embodiment, also described herein are methods of treating mesothelioma in a patient in need thereof, comprising administering to the patient an effective amount of the compound of Formula (I)

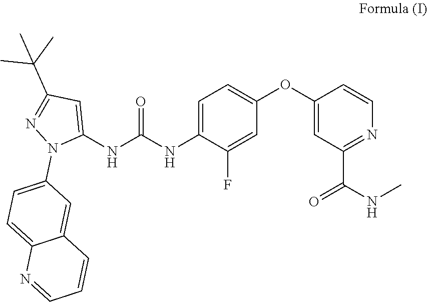 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In one embodiment, provided herein is a method of treating mesothelioma in a patient in need thereof, comprising administering to the patient about 50 mg to about 200 mg of the compound of Formula (I)

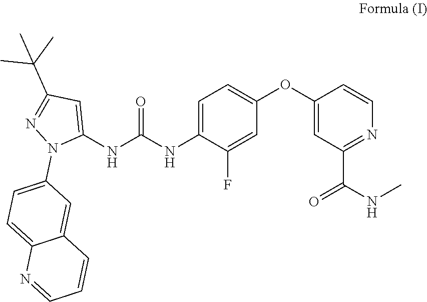 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof per day.

In another embodiment, provided herein is a method for achieving at least a partial response in a patient having mesothelioma, comprising administering to the patient an effective amount of the compound of Formula (I)

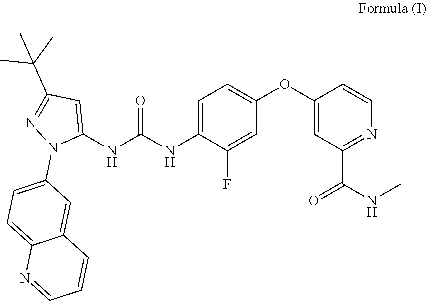 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In yet another embodiment, provided herein is a method for achieving a stable disease condition in a patient having mesothelioma, comprising to the patient administering the compound of Formula (I)

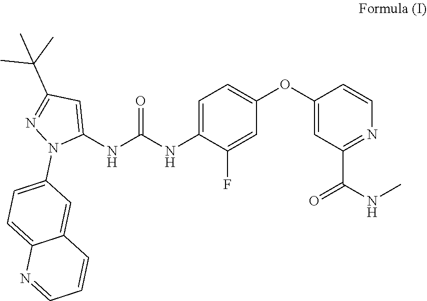 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

Also provided herein is a method of treating ovarian cancer (for example, a platinum-resistant ovarian cancer) in a patient in need thereof, comprising administering to the patient an effective amount of the compound of Formula (I)

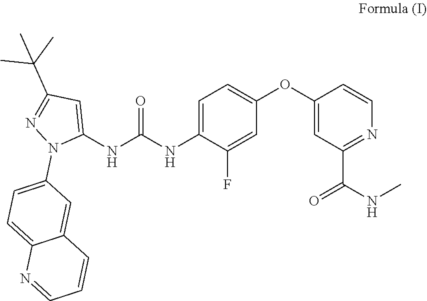 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In another embodiment, provided herein is a method for achieving at least a partial response in a patient having ovarian cancer (for example, a platinum-resistant ovarian cancer) in a patient in need thereof, comprising administering to the patient an effective amount of the compound of Formula (I)

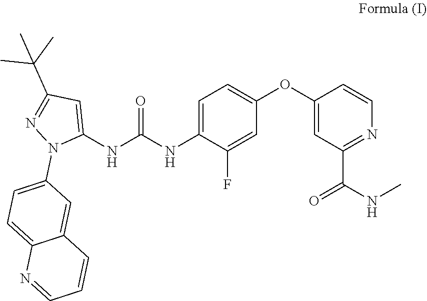 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In another embodiment, provided herein is a method for achieving at least a partial response in a patient having ovarian cancer (for example, a platinum-resistant ovarian cancer) in a patient in need thereof, comprising administering to the patient an effective amount of the compound of Formula (I)

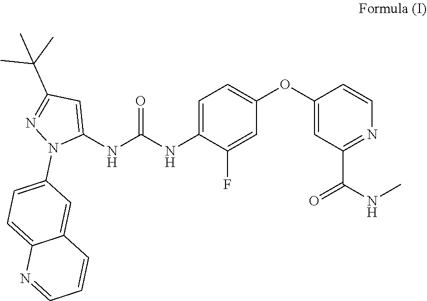 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In yet another embodiment, provided herein is a method for treating a patient having pancreatic ductal adenocarcinoma, comprising to the patient administering the compound of Formula (I)

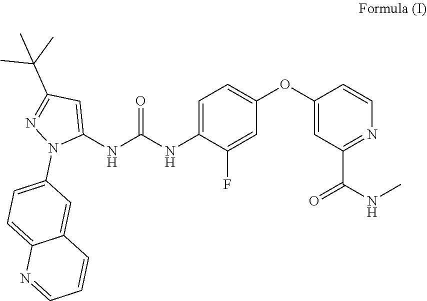 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In yet another embodiment, provided herein is a method for treating a patient having lung cancer, comprising to the patient administering the compound of Formula (I)

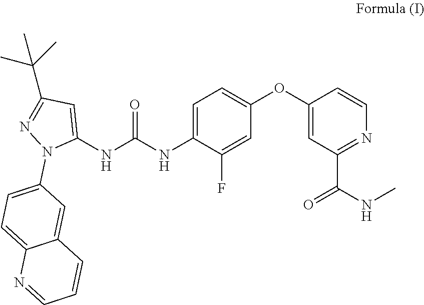 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

### BRIEF DESCRIPTION OF THE DRAWINGS

FIG. 1 shows the TEK/ANG1/Ang2 gene expression in exemplary mesothelioma PDx models studied in a combination efficacy study of rebastinib and carboplatin.

FIG. 2 shows the VEGF/CSF1/CSF1R gene expression in exemplary mesothelioma PDx models studied in a combination efficacy study of rebastinib and carboplatin.

FIG. 3A shows the tumor growth inhibition in exemplary evaluated mesothelioma PDx models using various dosing of drugs and drug combinations. FIG. 3B depicts dosing information for an exemplary model in this tumor growth inhibition study.

FIG. 4 and FIG. 5 demonstrate tumor volume changes in exemplary mesothelioma PDx models after administering vehicle, rebastinib, a combination of carboplatin and pemetrexed, and a combination of rebastinib, pemetrexed, and carboplatin.

FIG. 6 depicts exemplary RECIST v1.1 assessments of target lesions in two stage IV endometrial cancer patients with corresponding anticancer history listed.

FIG. 7 shows objective response rate (ORR) data for the mITT population by Subgroup Analyses in the mITT population of an exemplary endometrial cancer cohort treated with the compound of Formula (I) and paclitaxel.

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

The present disclosure provides methods of treating disorders that comprise administering to a patient in need thereof the compound of Formula (I)

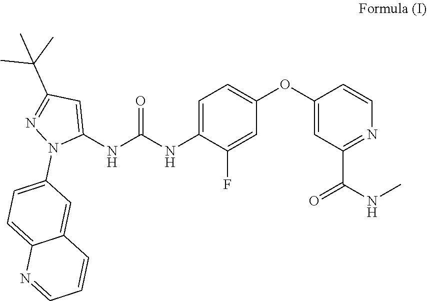 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof. The compound of Formula (I) is also referred to herein as “rebastinib.” Rebastinib and pharmaceutically acceptable salts thereof act as inhibitors of tunica interna endothelial cell kinase-2 (TIE2 kinase).

### Definitions

As used herein, a “combination therapy” is a treatment that includes the administration of two or more therapeutic agents, e.g., rebastinib or pharmaceutically acceptable salt thereof in combination with an additional therapeutic agents such as paclitaxel, carboplatin, cisplatin, gemcitabine, taxane therapy including nab-abraxane, FOLFOXIRI, FOLFIRI, anti-EGFR therapy, anti-HER pathway therapy, ALK kinase inhibitor therapy, RAF kinase inhibitor therapy, anti-VEGF therapy, anti-PD1 or anti-PDL1 therapy, or anti-CTLA4 therapy, to a patient.

“Individual,” “patient,” or “subject” are used interchangeably herein and include any animal, including mammals, including mice, rats, other rodents, rabbits, dogs, cats, swine, cattle, sheep, horses, or primates, and humans. The compounds described herein can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods described herein is desirably a mammal in which treatment of a disorder described herein is desired, such as a human.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

As used herein, “treating” includes any effect, e.g., lessening, reducing, modulating, or eliminating, that results in the improvement of the condition, disease, disorder and the like.

The terms “partial response,” “complete response,” “stable disease,” and “progressive disease” are used herein in accordance with their definitions provided by the Response Evaluation Criteria in Solid Tumors (RECIST) Version 1.1. “Stable disease condition” and “progressive disease condition” refer to the condition of patients who have achieved stable disease and progressive disease, respectively, as a result of the present methods described herein.

In the present specification, the term “effective amount” means the amount of the subject compound that will elicit the biological or medical response of a tissue, system or animal, (e.g. mammal or human) that is being sought by the researcher, veterinarian, medical doctor or other clinician.

### Pharmaceutical Compositions

A compound described herein, e.g., rebastinib or pharmaceutically acceptable salt thereof, or paclitaxel, can be formulated as a pharmaceutical composition using a pharmaceutically acceptable carrier and administered by a variety of routes. In some embodiments, such compositions are for oral administration. In some embodiments, a pharmaceutical composition comprising rebastinib or pharmaceutically acceptable salt thereof is formulated for oral administration. In some embodiments, such compositions are for parenteral (by injection) administration. In some embodiments, such compositions are for transdermal administration. In some embodiments, such compositions are for intravenous (IV) administration. In some embodiments, a pharmaceutical composition comprising paclitaxel or carboplatin is formulated for intravenous administration. In some embodiments, a pharmaceutical composition comprising paclitaxel is formulated for intraperitoneal administration (e.g., nanoparticulate paclitaxel, e.g., NANOTAX®). In some embodiments, such compositions are for intramuscular (IM) administration. Such pharmaceutical compositions and processes for preparing them are well known in the art. See, e.g., REMINGTON: THE SCIENCE AND PRACTICE OF PHARMACY (A. Gennaro, et al., eds., 19^{th} ed., Mack Publishing Co., 1995).

### Methods of Treatment

The present disclosure provides methods of treating disorders in patients in need thereof, comprising administering to these patients rebastinib or a pharmaceutically acceptable salt thereof. Exemplary disorders that can be treated by the present methods include but are not limited to gynecologic carcinosarcomas, endometrial adenocarcinomas, breast cancers, ovarian cancers (e.g., platinum-resistant ovarian cancers), mesotheliomas, pancreatic ductal adenocarcinomas, and lung cancers. In some embodiments, the disorders described herein are each characterized by a tumor microenvironment comprising TIE2-expressing macrophages (TEMs).

In some embodiments, the therapeutic efficacy of the administered compounds, e.g., rebastinib or pharmaceutically acceptable salt thereof, is determined by the progression-free survival of the patient after independent radiologic review using Response Evaluation Criteria in Solid Tumors (RECIST). In some embodiments, the therapeutic efficacy of the administered compounds, e.g., rebastinib or pharmaceutically acceptable salt thereof, is determined by the Objective Response Rate (ORR), Time to Tumor Progression (TTP) or Overall Survival (OS) of the patient after independent radiologic review using RECIST. In some embodiments, the therapeutic efficacy of the administered compounds, e.g., rebastinib or pharmaceutically acceptable salt thereof, is determined by the progression-free survival of the patient based on investigator assessment. In some embodiments, the therapeutic efficacy of the administered compounds, e.g., rebastinib or pharmaceutically acceptable salt thereof, is determined by the quality of life of the patient in accordance with European Organisation for Research and Treatment of Cancer Quality of Life Questionnaire for Cancer 30-item (EORTC-QLQ-C30) and the EuroQol 5-Dimension 5-Level (EQ-5D-5L) questionnaires. In some embodiments, the therapeutic efficacy of the administered compounds, e.g., rebastinib or pharmaceutically acceptable salt thereof, is determined by the disease control rate of the patient. In some embodiments, the therapeutic efficacy of the administered compounds, e.g., rebastinib or pharmaceutically acceptable salt thereof, is determined by the duration of response of the patient.

### Gynecologic Carcinosarcomas

Gynecologic carcinosarcomas (previously called malignant mixed Müllerian tumors [MMTs]) are rare and aggressive cancers that are dedifferentiated (metaplastic) carcinomas comprised of carcinomatous and sarcomatous elements arising from a single malignant epithelial clone.

Provided herein, inter alia, are methods of treating a gynecologic carcinosarcoma in a patient in need thereof, comprising administering to the patient an effective amount of the compound of Formula (I):

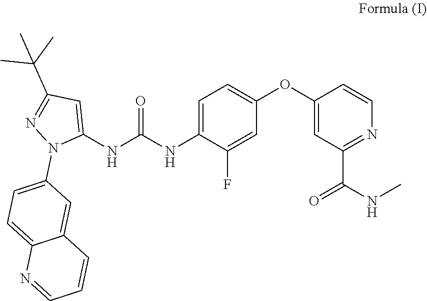 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof. The compound of Formula (I) may be administered in combination with one or more additional therapeutic agents, such as paclitaxel or another therapeutic agent described herein.

In one embodiment, provided herein is a method of treating a gynecologic carcinosarcoma in a patient that has previously received at least one line of treatment for the gynecologic carcinosarcoma, and is in need thereof, comprising administering to the patient an effective amount of the compound of Formula (I):

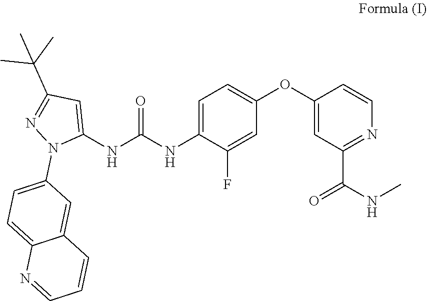 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In another embodiment, described herein is a method of treating a gynecologic carcinosarcoma in a patient that has previously received at least one line of treatment for the gynecologic carcinosarcoma, and is in need thereof, comprising administering to the patient an effective amount of: a) the compound of Formula (I)

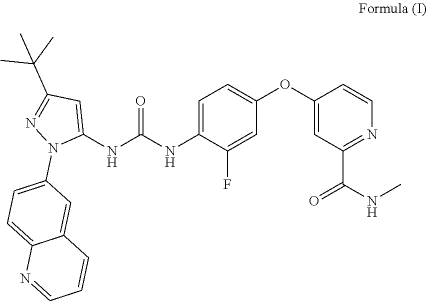 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof; and b) paclitaxel.

In some embodiments, the gynecologic carcinosarcoma is advanced gynecologic carcinosarcoma. In some embodiments, the gynecologic carcinosarcoma is a metastatic gynecologic carcinosarcoma. In some embodiments, the gynecologic carcinosarcoma is an endometrial carcinosarcoma. In some embodiments, the gynecologic carcinosarcoma is an ovarian carcinosarcoma. In some embodiments, the gynecologic carcinosarcoma is a vaginal carcinosarcoma. In some embodiments, the gynecologic carcinosarcoma is a cervical carcinosarcoma. In some embodiments, the gynecologic carcinosarcoma is of a homologous type or heterologous type.

In another embodiment, a method of treating a gynecologic carcinosarcoma described herein further comprises histologically determining if the gynecologic carcinosarcoma is advanced, persistent or recurrent. After several cycles (e.g., 4 to 6 cycles) of treatment with the compound of Formula (I) or pharmaceutically acceptable salt thereof and the paclitaxel, or after occurrence of toxicity, the method can further comprise administering to the patient an effective amount of the compound of Formula (I) or pharmaceutically acceptable salt thereof until disease progression or toxicity in the patient occurs.

In some embodiments, the methods of treating a gynecologic carcinosarcoma described herein comprise administering to a patient at least one line of treatment selected from the group consisting of a chemotherapy treatment, a checkpoint inhibitor treatment, a tyrosine-kinase inhibitor treatment, and a PARP-inhibitor treatment. In some embodiments, the chemotherapy treatment comprises a chemotherapeutic agent selected from the group consisting of paclitaxel, paclitaxel protein-bound particles for injectable suspension, eribulin, docetaxel, ixabepilone, vincristine, vinorelbine, cisplatin, carboplatin, oxaliplatin, cyclophosphamide, ifosfamide, temozolomide, doxorubicin, pegylated liposomal doxorubicin, daunorubicin, idarubicin, epirubicin, 5-fluorouracil, capecitabine, cytarabine, decitabine, 5-aza cytadine, gemcitabine, and methotrexate. In some embodiments, the checkpoint inhibitor treatment comprises an anti-PD1 therapeutic (an anti-PD1 antibody including pembrolizumab and nivolumab) or an anti-PD-L1 therapeutic (atezolizumab, durvalumab, BMS-936559, and avelumab). In some embodiments, the tyrosine-kinase inhibitor treatment comprises a tyrosine kinase inhibitor selected from the group consisting of afatinib, axitinib, binimetinib, bosutinib, cabozantinib, cediranib, ceritinib, cobimetinib, crizotinib, dabrafenib, dasatinib, erlotinib, everolimus, gefitinib, ibrutinib, imatinib, lapatinib, lenvatinib, lestaurtinib, nilotinib, nintedanib, palbociclib, pazopanib, ponatinib, regorafenib, ruxolitinib, semananib, sirolimus, sorafenib, sunitinib, temsirolimus, tofacitinib, trametinib, vandetanib, and vemurafenib. In some embodiments, the at least one line of treatment is a kinase inhibitor selected from the group consisting of erlotinib, gefitinib, lapatanib, lenvatinib, everolimus, temsirolimus, LY2835219, LEE011, PD 0332991, crizotinib, cabozantinib, sunitinib, pazopanib, sorafenib, regorafenib, axitinib, dasatinib, imatinib, nilotinib, vemurafenib, dabrafenib, trametinib, idelalisib, and quizartinib.

In some embodiments, the combination agent is an anti-VEGF or an anti-ANG biological including but not limited to bevacizumab, AMG386, or the bispecific anti-ANG+anti-VEGF agent RG7716.

In some embodiments, the PARP-inhibitor treatment comprises a PARP inhibitor selected from the group consisting of neraparib, olaparib, and rucaparib.

In some embodiments, the chemotherapy treatment comprises paclitaxel and/or carboplatin. In some embodiments, the chemotherapy treatment comprises paclitaxel and carboplatin.

A patient treated by the methods of treating a gynecologic carcinosarcoma described herein can achieve a partial response condition or a stable disease condition. In some embodiments, after 28 days or more of administering the compound of Formula (I) or pharmaceutically acceptable salt to the patient, the patient has achieved a partial response condition or a stable disease condition.

In embodiments of the methods of treating a gynecologic carcinosarcoma described herein, the compound of Formula (I) or pharmaceutically acceptable salt thereof can be orally administered to the patient in need thereof. In certain embodiments, the compound of Formula (I) or pharmaceutically acceptable salt thereof is administered to the patient daily. In some embodiments, the method comprises administering to the patient about 50 mg to about 200 mg of the compound of Formula (I) or pharmaceutically acceptable salt thereof per day. In some embodiments, the method comprises administering to the patient about 50 mg to about 150 mg of the compound of Formula (I) or pharmaceutically acceptable salt thereof per day. In some embodiments, the method comprises administering to the patient about 50 mg to about 100 mg of the compound of Formula (I) or pharmaceutically acceptable salt thereof per day. In some embodiments, the method comprises administering to the patient about 60 mg to about 200 mg of the compound of Formula (I) or pharmaceutically acceptable salt thereof per day. In some embodiments, the method comprises administering to the patient about 60 mg to about 150 mg of the compound of Formula (I) or pharmaceutically acceptable salt thereof per day. In some embodiments, the method comprises administering to the patient about 60 mg to about 100 mg of the compound of Formula (I) or pharmaceutically acceptable salt thereof per day. In some embodiments, the method comprises administering to the patient about 70 mg to about 200 mg of the compound of Formula (I) or pharmaceutically acceptable salt thereof per day. In some embodiments, the method comprises administering to the patient about 70 mg to about 150 mg of the compound of Formula (I) or pharmaceutically acceptable salt thereof per day. In some embodiments, the method comprises administering to the patient about 70 mg to about 100 mg of the compound of Formula (I) or pharmaceutically acceptable salt thereof per day. In some embodiments, the method comprises administering to the patient about 80 mg to about 200 mg of the compound of Formula (I) or pharmaceutically acceptable salt thereof per day. In some embodiments, the method comprises administering to the patient about 80 mg to about 150 mg of the compound of Formula (I) or pharmaceutically acceptable salt thereof per day. In some embodiments, the method comprises administering to the patient about 80 mg to about 100 mg of the compound of Formula (I) or pharmaceutically acceptable salt thereof per day. In some embodiments, the method comprises administering to the patient about 90 mg to about 200 mg of the compound of Formula (I) or pharmaceutically acceptable salt thereof per day. In some embodiments, the method comprises administering to the patient about 90 mg to about 150 mg of the compound of Formula (I) or pharmaceutically acceptable salt thereof per day. In some embodiments, the method comprises administering to the patient about 90 mg to about 100 mg of the compound of Formula (I) or pharmaceutically acceptable salt thereof per day. In some embodiments, the method comprises administering to the patient about 100 mg to about 200 mg of the compound of Formula (I) or pharmaceutically acceptable salt thereof per day. In some embodiments, the method comprises administering to the patient about 100 mg to about 150 mg of the compound of Formula (I) or pharmaceutically acceptable salt thereof per day.

In some embodiments, the method comprises administering to the patient about 50 mg of the compound of Formula (I) or pharmaceutically acceptable salt thereof per day. In some embodiments, the method comprises administering to the patient about 55 mg of the compound of Formula (I) or pharmaceutically acceptable salt thereof per day. In some embodiments, the method comprises administering to the patient about 60 mg of the compound of Formula (I) or pharmaceutically acceptable salt thereof per day. In some embodiments, the method comprises administering to the patient about 65 mg of the compound of Formula (I) or pharmaceutically acceptable salt thereof per day. In some embodiments, the method comprises administering to the patient about 70 mg of the compound of Formula (I) or pharmaceutically acceptable salt thereof per day. In some embodiments, the method comprises administering to the patient about 75 mg of the compound of Formula (I) or pharmaceutically acceptable salt thereof per day. In some embodiments, the method comprises administering to the patient about 80 mg of the compound of Formula (I) or pharmaceutically acceptable salt thereof per day. In some embodiments, the method comprises administering to the patient about 85 mg of the compound of Formula (I) or pharmaceutically acceptable salt thereof per day. In some embodiments, the method comprises administering to the patient about 90 mg of the compound of Formula (I) or pharmaceutically acceptable salt thereof per day. In some embodiments, the method comprises administering to the patient about 95 mg of the compound of Formula (I) or pharmaceutically acceptable salt thereof per day. In some embodiments, the method comprises administering to the patient about 100 mg of the compound of Formula (I) or pharmaceutically acceptable salt thereof per day. In some embodiments, the method comprises administering to the patient about 105 mg of the compound of Formula (I) or pharmaceutically acceptable salt thereof per day. In some embodiments, the method comprises administering to the patient about 110 mg of the compound of Formula (I) or pharmaceutically acceptable salt thereof per day. In some embodiments, the method comprises administering to the patient about 115 mg of the compound of Formula (I) or pharmaceutically acceptable salt thereof per day. In some embodiments, the method comprises administering to the patient about 120 mg of the compound of Formula (I) or pharmaceutically acceptable salt thereof per day. In some embodiments, the method comprises administering to the patient about 125 mg of the compound of Formula (I) or pharmaceutically acceptable salt thereof per day. In some embodiments, the method comprises administering to the patient about 130 mg of the compound of Formula (I) or pharmaceutically acceptable salt thereof per day. In some embodiments, the method comprises administering to the patient about 135 mg of the compound of Formula (I) or pharmaceutically acceptable salt thereof per day. In some embodiments, the method comprises administering to the patient about 140 mg of the compound of Formula (I) or pharmaceutically acceptable salt thereof per day. In some embodiments, the method comprises administering to the patient about 145 mg of the compound of Formula (I) or pharmaceutically acceptable salt thereof per day. In some embodiments, the method comprises administering to the patient about 150 mg of the compound of Formula (I) or pharmaceutically acceptable salt thereof per day. In some embodiments, the method comprises administering to the patient about 155 mg of the compound of Formula (I) or pharmaceutically acceptable salt thereof per day. In some embodiments, the method comprises administering to the patient about 160 mg of the compound of Formula (I) or pharmaceutically acceptable salt thereof per day. In some embodiments, the method comprises administering to the patient about 165 mg of the compound of Formula (I) or pharmaceutically acceptable salt thereof per day. In some embodiments, the method comprises administering to the patient about 170 mg of the compound of Formula (I) or pharmaceutically acceptable salt thereof per day. In some embodiments, the method comprises administering to the patient about 175 mg of the compound of Formula (I) or pharmaceutically acceptable salt thereof per day. In some embodiments, the method comprises administering to the patient about 180 mg of the compound of Formula (I) or pharmaceutically acceptable salt thereof per day. In some embodiments, the method comprises administering to the patient about 185 mg of the compound of Formula (I) or pharmaceutically acceptable salt thereof per day. In some embodiments, the method comprises administering to the patient about 190 mg of the compound of Formula (I) or pharmaceutically acceptable salt thereof per day. In some embodiments, the method comprises administering to the patient about 195 mg of the compound of Formula (I) or pharmaceutically acceptable salt thereof per day. In some embodiments, the method comprises administering to the patient about 200 mg of the compound of Formula (I) or pharmaceutically acceptable salt thereof per day.

In some embodiments, the method comprises administering to the patient about 50 mg to about 200 mg of the compound of Formula (I) or pharmaceutically acceptable salt thereof once per day. In some embodiments, the method comprises administering to the patient about 50 mg to about 150 mg of the compound of Formula (I) or pharmaceutically acceptable salt thereof once per day. In some embodiments, the method comprises administering to the patient about 50 mg to about 100 mg of the compound of Formula (I) or pharmaceutically acceptable salt thereof once per day. In some embodiments, the method comprises administering to the patient about 60 mg to about 200 mg of the compound of Formula (I) or pharmaceutically acceptable salt thereof once per day. In some embodiments, the method comprises administering to the patient about 60 mg to about 150 mg of the compound of Formula (I) or pharmaceutically acceptable salt thereof once per day. In some embodiments, the method comprises administering to the patient about 60 mg to about 100 mg of the compound of Formula (I) or pharmaceutically acceptable salt thereof once per day. In some embodiments, the method comprises administering to the patient about 70 mg to about 200 mg of the compound of Formula (I) or pharmaceutically acceptable salt thereof once per day. In some embodiments, the method comprises administering to the patient about 70 mg to about 150 mg of the compound of Formula (I) or pharmaceutically acceptable salt thereof once per day. In some embodiments, the method comprises administering to the patient about 70 mg to about 100 mg of the compound of Formula (I) or pharmaceutically acceptable salt thereof once per day. In some embodiments, the method comprises administering to the patient about 80 mg to about 200 mg of the compound of Formula (I) or pharmaceutically acceptable salt thereof once per day. In some embodiments, the method comprises administering to the patient about 80 mg to about 150 mg of the compound of Formula (I) or pharmaceutically acceptable salt thereof once per day. In some embodiments, the method comprises administering to the patient about 80 mg to about 100 mg of the compound of Formula (I) or pharmaceutically acceptable salt thereof once per day. In some embodiments, the method comprises administering to the patient about 90 mg to about 200 mg of the compound of Formula (I) or pharmaceutically acceptable salt thereof once per day. In some embodiments, the method comprises administering to the patient about 90 mg to about 150 mg of the compound of Formula (I) or pharmaceutically acceptable salt thereof once per day. In some embodiments, the method comprises administering to the patient about 90 mg to about 100 mg of the compound of Formula (I) or pharmaceutically acceptable salt thereof once per day. In some embodiments, the method comprises administering to the patient about 100 mg to about 200 mg of the compound of Formula (I) or pharmaceutically acceptable salt thereof once per day. In some embodiments, the method comprises administering to the patient about 100 mg to about 150 mg of the compound of Formula (I) or pharmaceutically acceptable salt thereof once per day.

In some embodiments, the method comprises administering to the patient about 50 mg of the compound of Formula (I) or pharmaceutically acceptable salt thereof once per day. In some embodiments, the method comprises administering to the patient about 55 mg of the compound of Formula (I) or pharmaceutically acceptable salt thereof once per day. In some embodiments, the method comprises administering to the patient about 60 mg of the compound of Formula (I) or pharmaceutically acceptable salt thereof once per day. In some embodiments, the method comprises administering to the patient about 65 mg of the compound of Formula (I) or pharmaceutically acceptable salt thereof once per day. In some embodiments, the method comprises administering to the patient about 70 mg of the compound of Formula (I) or pharmaceutically acceptable salt thereof once per day. In some embodiments, the method comprises administering to the patient about 75 mg of the compound of Formula (I) or pharmaceutically acceptable salt thereof once per day. In some embodiments, the method comprises administering to the patient about 80 mg of the compound of Formula (I) or pharmaceutically acceptable salt thereof once per day. In some embodiments, the method comprises administering to the patient about 85 mg of the compound of Formula (I) or pharmaceutically acceptable salt thereof once per day. In some embodiments, the method comprises administering to the patient about 90 mg of the compound of Formula (I) or pharmaceutically acceptable salt thereof once per day. In some embodiments, the method comprises administering to the patient about 95 mg of the compound of Formula (I) or pharmaceutically acceptable salt thereof once per day. In some embodiments, the method comprises administering to the patient about 100 mg of the compound of Formula (I) or pharmaceutically acceptable salt thereof once per day. In some embodiments, the method comprises administering to the patient about 105 mg of the compound of Formula (I) or pharmaceutically acceptable salt thereof once per day. In some embodiments, the method comprises administering to the patient about 110 mg of the compound of Formula (I) or pharmaceutically acceptable salt thereof once per day. In some embodiments, the method comprises administering to the patient about 115 mg of the compound of Formula (I) or pharmaceutically acceptable salt thereof once per day. In some embodiments, the method comprises administering to the patient about 120 mg of the compound of Formula (I) or pharmaceutically acceptable salt thereof once per day. In some embodiments, the method comprises administering to the patient about 125 mg of the compound of Formula (I) or pharmaceutically acceptable salt thereof once per day. In some embodiments, the method comprises administering to the patient about 130 mg of the compound of Formula (I) or pharmaceutically acceptable salt thereof once per day. In some embodiments, the method comprises administering to the patient about 135 mg of the compound of Formula (I) or pharmaceutically acceptable salt thereof once per day. In some embodiments, the method comprises administering to the patient about 140 mg of the compound of Formula (I) or pharmaceutically acceptable salt thereof once per day. In some embodiments, the method comprises administering to the patient about 145 mg of the compound of Formula (I) or pharmaceutically acceptable salt thereof once per day. In some embodiments, the method comprises administering to the patient about 150 mg of the compound of Formula (I) or pharmaceutically acceptable salt thereof once per day. In some embodiments, the method comprises administering to the patient about 155 mg of the compound of Formula (I) or pharmaceutically acceptable salt thereof once per day. In some embodiments, the method comprises administering to the patient about 160 mg of the compound of Formula (I) or pharmaceutically acceptable salt thereof once per day. In some embodiments, the method comprises administering to the patient about 165 mg of the compound of Formula (I) or pharmaceutically acceptable salt thereof once per day. In some embodiments, the method comprises administering to the patient about 170 mg of the compound of Formula (I) or pharmaceutically acceptable salt thereof once per day. In some embodiments, the method comprises administering to the patient about 175 mg of the compound of Formula (I) or pharmaceutically acceptable salt thereof once per day. In some embodiments, the method comprises administering to the patient about 180 mg of the compound of Formula (I) or pharmaceutically acceptable salt thereof once per day. In some embodiments, the method comprises administering to the patient about 185 mg of the compound of Formula (I) or pharmaceutically acceptable salt thereof once per day. In some embodiments, the method comprises administering to the patient about 190 mg of the compound of Formula (I) or pharmaceutically acceptable salt thereof once per day. In some embodiments, the method comprises administering to the patient about 195 mg of the compound of Formula (I) or pharmaceutically acceptable salt thereof once per day. In some embodiments, the method comprises administering to the patient about 200 mg of the compound of Formula (I) or pharmaceutically acceptable salt thereof once per day.

In some embodiments, the method comprises administering to the patient about 50 mg to about 100 mg of the compound of Formula (I) or pharmaceutically acceptable salt thereof twice per day. In some embodiments, the method comprises administering to the patient about 60 mg to about 100 mg of the compound of Formula (I) or pharmaceutically acceptable salt thereof twice per day. In some embodiments, the method comprises administering to the patient about 70 mg to about 100 mg of the compound of Formula (I) or pharmaceutically acceptable salt thereof twice per day. In some embodiments, the method comprises administering to the patient about 80 mg to about 100 mg of the compound of Formula (I) or pharmaceutically acceptable salt thereof twice per day. In some embodiments, the method comprises administering to the patient about 90 mg to about 100 mg of the compound of Formula (I) or pharmaceutically acceptable salt thereof twice per day.

In some embodiments, the method comprises administering to the patient about 50 mg of the compound of Formula (I) or pharmaceutically acceptable salt thereof twice per day. In some embodiments, the method comprises administering to the patient about 55 mg of the compound of Formula (I) or pharmaceutically acceptable salt thereof twice per day. In some embodiments, the method comprises administering to the patient about 60 mg of the compound of Formula (I) or pharmaceutically acceptable salt thereof twice per day. In some embodiments, the method comprises administering to the patient about 65 mg of the compound of Formula (I) or pharmaceutically acceptable salt thereof twice per day. In some embodiments, the method comprises administering to the patient about 70 mg of the compound of Formula (I) or pharmaceutically acceptable salt thereof twice per day. In some embodiments, the method comprises administering to the patient about 75 mg of the compound of Formula (I) or pharmaceutically acceptable salt thereof twice per day. In some embodiments, the method comprises administering to the patient about 80 mg of the compound of Formula (I) or pharmaceutically acceptable salt thereof twice per day. In some embodiments, the method comprises administering to the patient about 85 mg of the compound of Formula (I) or pharmaceutically acceptable salt thereof twice per day. In some embodiments, the method comprises administering to the patient about 90 mg of the compound of Formula (I) or pharmaceutically acceptable salt thereof twice per day. In some embodiments, the method comprises administering to the patient about 95 mg of the compound of Formula (I) or pharmaceutically acceptable salt thereof twice per day. In some embodiments, the method comprises administering to the patient about 100 mg of the compound of Formula (I) or pharmaceutically acceptable salt thereof twice per day.

The compound of Formula (I) or pharmaceutically acceptable salt thereof may, in another embodiment, be administered to the patient on an intermittent basis in the methods of treating a gynecologic carcinosarcoma described herein. In some embodiments, the compound of Formula (I) or pharmaceutically acceptable salt thereof is administered the patient every other day. In some embodiments, the compound of Formula (I) or pharmaceutically acceptable salt thereof is administered the patient every other day. In some embodiments, the compound of Formula (I) or pharmaceutically acceptable salt thereof is administered the patient once per week. In some embodiments, the compound of Formula (I) or pharmaceutically acceptable salt thereof is administered the patient twice per week. In some embodiments, the compound of Formula (I) or pharmaceutically acceptable salt thereof is administered the patient three times per week.

In some embodiments, the method comprises administering to the patient about 50 mg to about 200 mg of the compound of Formula (I) or pharmaceutically acceptable salt thereof once a week. In some embodiments, the method comprises administering to the patient about 50 mg to about 150 mg of the compound of Formula (I) or pharmaceutically acceptable salt thereof once a week. In some embodiments, the method comprises administering to the patient about 50 mg to about 100 mg of the compound of Formula (I) or pharmaceutically acceptable salt thereof once a week. In some embodiments, the method comprises administering to the patient about 60 mg to about 200 mg of the compound of Formula (I) or pharmaceutically acceptable salt thereof once a week. In some embodiments, the method comprises administering to the patient about 60 mg to about 150 mg of the compound of Formula (I) or pharmaceutically acceptable salt thereof once a week. In some embodiments, the method comprises administering to the patient about 60 mg to about 100 mg of the compound of Formula (I) or pharmaceutically acceptable salt thereof once a week. In some embodiments, the method comprises administering to the patient about 70 mg to about 200 mg of the compound of Formula (I) or pharmaceutically acceptable salt thereof once a week. In some embodiments, the method comprises administering to the patient about 70 mg to about 150 mg of the compound of Formula (I) or pharmaceutically acceptable salt thereof once a week. In some embodiments, the method comprises administering to the patient about 70 mg to about 100 mg of the compound of Formula (I) or pharmaceutically acceptable salt thereof once a week. In some embodiments, the method comprises administering to the patient about 80 mg to about 200 mg of the compound of Formula (I) or pharmaceutically acceptable salt thereof once a week. In some embodiments, the method comprises administering to the patient about 80 mg to about 150 mg of the compound of Formula (I) or pharmaceutically acceptable salt thereof once a week. In some embodiments, the method comprises administering to the patient about 80 mg to about 100 mg of the compound of Formula (I) or pharmaceutically acceptable salt thereof once a week. In some embodiments, the method comprises administering to the patient about 90 mg to about 200 mg of the compound of Formula (I) or pharmaceutically acceptable salt thereof once a week. In some embodiments, the method comprises administering to the patient about 90 mg to about 150 mg of the compound of Formula (I) or pharmaceutically acceptable salt thereof once a week. In some embodiments, the method comprises administering to the patient about 90 mg to about 100 mg of the compound of Formula (I) or pharmaceutically acceptable salt thereof once a week. In some embodiments, the method comprises administering to the patient about 100 mg to about 200 mg of the compound of Formula (I) or pharmaceutically acceptable salt thereof once a week. In some embodiments, the method comprises administering to the patient about 100 mg to about 150 mg of the compound of Formula (I) or pharmaceutically acceptable salt thereof once a week.

In some embodiments, the method comprises administering to the patient about 50 mg of the compound of Formula (I) or pharmaceutically acceptable salt thereof once a week. In some embodiments, the method comprises administering to the patient about 55 mg of the compound of Formula (I) or pharmaceutically acceptable salt thereof once a week. In some embodiments, the method comprises administering to the patient about 60 mg of the compound of Formula (I) or pharmaceutically acceptable salt thereof once a week. In some embodiments, the method comprises administering to the patient about 65 mg of the compound of Formula (I) or pharmaceutically acceptable salt thereof once a week. In some embodiments, the method comprises administering to the patient about 70 mg of the compound of Formula (I) or pharmaceutically acceptable salt thereof once a week. In some embodiments, the method comprises administering to the patient about 75 mg of the compound of Formula (I) or pharmaceutically acceptable salt thereof once a week. In some embodiments, the method comprises administering to the patient about 80 mg of the compound of Formula (I) or pharmaceutically acceptable salt thereof once a week. In some embodiments, the method comprises administering to the patient about 85 mg of the compound of Formula (I) or pharmaceutically acceptable salt thereof once a week. In some embodiments, the method comprises administering to the patient about 90 mg of the compound of Formula (I) or pharmaceutically acceptable salt thereof once a week. In some embodiments, the method comprises administering to the patient about 95 mg of the compound of Formula (I) or pharmaceutically acceptable salt thereof once a week. In some embodiments, the method comprises administering to the patient about 100 mg of the compound of Formula (I) or pharmaceutically acceptable salt thereof once a week. In some embodiments, the method comprises administering to the patient about 105 mg of the compound of Formula (I) or pharmaceutically acceptable salt thereof once a week. In some embodiments, the method comprises administering to the patient about 110 mg of the compound of Formula (I) or pharmaceutically acceptable salt thereof once a week. In some embodiments, the method comprises administering to the patient about 115 mg of the compound of Formula (I) or pharmaceutically acceptable salt thereof once a week. In some embodiments, the method comprises administering to the patient about 120 mg of the compound of Formula (I) or pharmaceutically acceptable salt thereof once a week. In some embodiments, the method comprises administering to the patient about 125 mg of the compound of Formula (I) or pharmaceutically acceptable salt thereof once a week. In some embodiments, the method comprises administering to the patient about 130 mg of the compound of Formula (I) or pharmaceutically acceptable salt thereof once a week. In some embodiments, the method comprises administering to the patient about 135 mg of the compound of Formula (I) or pharmaceutically acceptable salt thereof once a week. In some embodiments, the method comprises administering to the patient about 140 mg of the compound of Formula (I) or pharmaceutically acceptable salt thereof once a week. In some embodiments, the method comprises administering to the patient about 145 mg of the compound of Formula (I) or pharmaceutically acceptable salt thereof once a week. In some embodiments, the method comprises administering to the patient about 150 mg of the compound of Formula (I) or pharmaceutically acceptable salt thereof once a week. In some embodiments, the method comprises administering to the patient about 155 mg of the compound of Formula (I) or pharmaceutically acceptable salt thereof once a week. In some embodiments, the method comprises administering to the patient about 160 mg of the compound of Formula (I) or pharmaceutically acceptable salt thereof once a week. In some embodiments, the method comprises administering to the patient about 165 mg of the compound of Formula (I) or pharmaceutically acceptable salt thereof once a week. In some embodiments, the method comprises administering to the patient about 170 mg of the compound of Formula (I) or pharmaceutically acceptable salt thereof once a week. In some embodiments, the method comprises administering to the patient about 175 mg of the compound of Formula (I) or pharmaceutically acceptable salt thereof once a week. In some embodiments, the method comprises administering to the patient about 180 mg of the compound of Formula (I) or pharmaceutically acceptable salt thereof once a week. In some embodiments, the method comprises administering to the patient about 185 mg of the compound of Formula (I) or pharmaceutically acceptable salt thereof once a week. In some embodiments, the method comprises administering to the patient about 190 mg of the compound of Formula (I) or pharmaceutically acceptable salt thereof once a week. In some embodiments, the method comprises administering to the patient about 195 mg of the compound of Formula (I) or pharmaceutically acceptable salt thereof once a week. In some embodiments, the method comprises administering to the patient about 200 mg of the compound of Formula (I) or pharmaceutically acceptable salt thereof once a week.

In some embodiments, the method comprises administering to the patient about 50 mg to about 200 mg of the compound of Formula (I) or pharmaceutically acceptable salt thereof twice a week. In some embodiments, the method comprises administering to the patient about 50 mg to about 150 mg of the compound of Formula (I) or pharmaceutically acceptable salt thereof twice a week. In some embodiments, the method comprises administering to the patient about 50 mg to about 100 mg of the compound of Formula (I) or pharmaceutically acceptable salt thereof twice a week. In some embodiments, the method comprises administering to the patient about 60 mg to about 200 mg of the compound of Formula (I) or pharmaceutically acceptable salt thereof twice a week. In some embodiments, the method comprises administering to the patient about 60 mg to about 150 mg of the compound of Formula (I) or pharmaceutically acceptable salt thereof twice a week. In some embodiments, the method comprises administering to the patient about 60 mg to about 100 mg of the compound of Formula (I) or pharmaceutically acceptable salt thereof twice a week. In some embodiments, the method comprises administering to the patient about 70 mg to about 200 mg of the compound of Formula (I) or pharmaceutically acceptable salt thereof twice a week. In some embodiments, the method comprises administering to the patient about 70 mg to about 150 mg of the compound of Formula (I) or pharmaceutically acceptable salt thereof twice a week. In some embodiments, the method comprises administering to the patient about 70 mg to about 100 mg of the compound of Formula (I) or pharmaceutically acceptable salt thereof twice a week. In some embodiments, the method comprises administering to the patient about 80 mg to about 200 mg of the compound of Formula (I) or pharmaceutically acceptable salt thereof twice a week. In some embodiments, the method comprises administering to the patient about 80 mg to about 150 mg of the compound of Formula (I) or pharmaceutically acceptable salt thereof twice a week. In some embodiments, the method comprises administering to the patient about 80 mg to about 100 mg of the compound of Formula (I) or pharmaceutically acceptable salt thereof twice a week. In some embodiments, the method comprises administering to the patient about 90 mg to about 200 mg of the compound of Formula (I) or pharmaceutically acceptable salt thereof twice a week. In some embodiments, the method comprises administering to the patient about 90 mg to about 150 mg of the compound of Formula (I) or pharmaceutically acceptable salt thereof twice a week. In some embodiments, the method comprises administering to the patient about 90 mg to about 100 mg of the compound of Formula (I) or pharmaceutically acceptable salt thereof twice a week. In some embodiments, the method comprises administering to the patient about 100 mg to about 200 mg of the compound of Formula (I) or pharmaceutically acceptable salt thereof twice a week. In some embodiments, the method comprises administering to the patient about 100 mg to about 150 mg of the compound of Formula (I) or pharmaceutically acceptable salt thereof twice a week.

In some embodiments, the method comprises administering to the patient about 50 mg of the compound of Formula (I) or pharmaceutically acceptable salt thereof twice a week. In some embodiments, the method comprises administering to the patient about 55 mg of the compound of Formula (I) or pharmaceutically acceptable salt thereof twice a week. In some embodiments, the method comprises administering to the patient about 60 mg of the compound of Formula (I) or pharmaceutically acceptable salt thereof twice a week. In some embodiments, the method comprises administering to the patient about 65 mg of the compound of Formula (I) or pharmaceutically acceptable salt thereof twice a week. In some embodiments, the method comprises administering to the patient about 70 mg of the compound of Formula (I) or pharmaceutically acceptable salt thereof twice a week. In some embodiments, the method comprises administering to the patient about 75 mg of the compound of Formula (I) or pharmaceutically acceptable salt thereof twice a week. In some embodiments, the method comprises administering to the patient about 80 mg of the compound of Formula (I) or pharmaceutically acceptable salt thereof twice a week. In some embodiments, the method comprises administering to the patient about 85 mg of the compound of Formula (I) or pharmaceutically acceptable salt thereof twice a week. In some embodiments, the method comprises administering to the patient about 90 mg of the compound of Formula (I) or pharmaceutically acceptable salt thereof twice a week. In some embodiments, the method comprises administering to the patient about 95 mg of the compound of Formula (I) or pharmaceutically acceptable salt thereof twice a week. In some embodiments, the method comprises administering to the patient about 100 mg of the compound of Formula (I) or pharmaceutically acceptable salt thereof twice a week. In some embodiments, the method comprises administering to the patient about 105 mg of the compound of Formula (I) or pharmaceutically acceptable salt thereof twice a week. In some embodiments, the method comprises administering to the patient about 110 mg of the compound of Formula (I) or pharmaceutically acceptable salt thereof twice a week. In some embodiments, the method comprises administering to the patient about 115 mg of the compound of Formula (I) or pharmaceutically acceptable salt thereof twice a week. In some embodiments, the method comprises administering to the patient about 120 mg of the compound of Formula (I) or pharmaceutically acceptable salt thereof twice a week. In some embodiments, the method comprises administering to the patient about 125 mg of the compound of Formula (I) or pharmaceutically acceptable salt thereof twice a week. In some embodiments, the method comprises administering to the patient about 130 mg of the compound of Formula (I) or pharmaceutically acceptable salt thereof twice a week. In some embodiments, the method comprises administering to the patient about 135 mg of the compound of Formula (I) or pharmaceutically acceptable salt thereof twice a week. In some embodiments, the method comprises administering to the patient about 140 mg of the compound of Formula (I) or pharmaceutically acceptable salt thereof twice a week. In some embodiments, the method comprises administering to the patient about 145 mg of the compound of Formula (I) or pharmaceutically acceptable salt thereof twice a week. In some embodiments, the method comprises administering to the patient about 150 mg of the compound of Formula (I) or pharmaceutically acceptable salt thereof twice a week. In some embodiments, the method comprises administering to the patient about 155 mg of the compound of Formula (I) or pharmaceutically acceptable salt thereof twice a week. In some embodiments, the method comprises administering to the patient about 160 mg of the compound of Formula (I) or pharmaceutically acceptable salt thereof twice a week. In some embodiments, the method comprises administering to the patient about 165 mg of the compound of Formula (I) or pharmaceutically acceptable salt thereof twice a week. In some embodiments, the method comprises administering to the patient about 170 mg of the compound of Formula (I) or pharmaceutically acceptable salt thereof twice a week. In some embodiments, the method comprises administering to the patient about 175 mg of the compound of Formula (I) or pharmaceutically acceptable salt thereof twice a week. In some embodiments, the method comprises administering to the patient about 180 mg of the compound of Formula (I) or pharmaceutically acceptable salt thereof twice a week. In some embodiments, the method comprises administering to the patient about 185 mg of the compound of Formula (I) or pharmaceutically acceptable salt thereof twice a week. In some embodiments, the method comprises administering to the patient about 190 mg of the compound of Formula (I) or pharmaceutically acceptable salt thereof twice a week. In some embodiments, the method comprises administering to the patient about 195 mg of the compound of Formula (I) or pharmaceutically acceptable salt thereof twice a week. In some embodiments, the method comprises administering to the patient about 200 mg of the compound of Formula (I) or pharmaceutically acceptable salt thereof twice a week.

In some embodiments, the method comprises administering to the patient about 50 mg to about 200 mg of the compound of Formula (I) or pharmaceutically acceptable salt thereof three times a week. In some embodiments, the method comprises administering to the patient about 50 mg to about 150 mg of the compound of Formula (I) or pharmaceutically acceptable salt thereof three times a week. In some embodiments, the method comprises administering to the patient about 50 mg to about 100 mg of the compound of Formula (I) or pharmaceutically acceptable salt thereof three times a week. In some embodiments, the method comprises administering to the patient about 60 mg to about 200 mg of the compound of Formula (I) or pharmaceutically acceptable salt thereof three times a week. In some embodiments, the method comprises administering to the patient about 60 mg to about 150 mg of the compound of Formula (I) or pharmaceutically acceptable salt thereof three times a week. In some embodiments, the method comprises administering to the patient about 60 mg to about 100 mg of the compound of Formula (I) or pharmaceutically acceptable salt thereof three times a week. In some embodiments, the method comprises administering to the patient about 70 mg to about 200 mg of the compound of Formula (I) or pharmaceutically acceptable salt thereof three times a week. In some embodiments, the method comprises administering to the patient about 70 mg to about 150 mg of the compound of Formula (I) or pharmaceutically acceptable salt thereof three times a week. In some embodiments, the method comprises administering to the patient about 70 mg to about 100 mg of the compound of Formula (I) or pharmaceutically acceptable salt thereof three times a week. In some embodiments, the method comprises administering to the patient about 80 mg to about 200 mg of the compound of Formula (I) or pharmaceutically acceptable salt thereof three times a week. In some embodiments, the method comprises administering to the patient about 80 mg to about 150 mg of the compound of Formula (I) or pharmaceutically acceptable salt thereof three times a week. In some embodiments, the method comprises administering to the patient about 80 mg to about 100 mg of the compound of Formula (I) or pharmaceutically acceptable salt thereof three times a week. In some embodiments, the method comprises administering to the patient about 90 mg to about 200 mg of the compound of Formula (I) or pharmaceutically acceptable salt thereof three times a week. In some embodiments, the method comprises administering to the patient about 90 mg to about 150 mg of the compound of Formula (I) or pharmaceutically acceptable salt thereof three times a week. In some embodiments, the method comprises administering to the patient about 90 mg to about 100 mg of the compound of Formula (I) or pharmaceutically acceptable salt thereof three times a week. In some embodiments, the method comprises administering to the patient about 100 mg to about 200 mg of the compound of Formula (I) or pharmaceutically acceptable salt thereof three times a week. In some embodiments, the method comprises administering to the patient about 100 mg to about 150 mg of the compound of Formula (I) or pharmaceutically acceptable salt thereof three times a week.

In some embodiments, the method comprises administering to the patient about 50 mg of the compound of Formula (I) or pharmaceutically acceptable salt thereof three times a week. In some embodiments, the method comprises administering to the patient about 55 mg of the compound of Formula (I) or pharmaceutically acceptable salt thereof three times a week. In some embodiments, the method comprises administering to the patient about 60 mg of the compound of Formula (I) or pharmaceutically acceptable salt thereof three times a week. In some embodiments, the method comprises administering to the patient about 65 mg of the compound of Formula (I) or pharmaceutically acceptable salt thereof three times a week. In some embodiments, the method comprises administering to the patient about 70 mg of the compound of Formula (I) or pharmaceutically acceptable salt thereof three times a week. In some embodiments, the method comprises administering to the patient about 75 mg of the compound of Formula (I) or pharmaceutically acceptable salt thereof three times a week. In some embodiments, the method comprises administering to the patient about 80 mg of the compound of Formula (I) or pharmaceutically acceptable salt thereof three times a week. In some embodiments, the method comprises administering to the patient about 85 mg of the compound of Formula (I) or pharmaceutically acceptable salt thereof three times a week. In some embodiments, the method comprises administering to the patient about 90 mg of the compound of Formula (I) or pharmaceutically acceptable salt thereof three times a week. In some embodiments, the method comprises administering to the patient about 95 mg of the compound of Formula (I) or pharmaceutically acceptable salt thereof three times a week. In some embodiments, the method comprises administering to the patient about 100 mg of the compound of Formula (I) or pharmaceutically acceptable salt thereof three times a week. In some embodiments, the method comprises administering to the patient about 105 mg of the compound of Formula (I) or pharmaceutically acceptable salt thereof three times a week. In some embodiments, the method comprises administering to the patient about 110 mg of the compound of Formula (I) or pharmaceutically acceptable salt thereof three times a week. In some embodiments, the method comprises administering to the patient about 115 mg of the compound of Formula (I) or pharmaceutically acceptable salt thereof three times a week. In some embodiments, the method comprises administering to the patient about 120 mg of the compound of Formula (I) or pharmaceutically acceptable salt thereof three times a week. In some embodiments, the method comprises administering to the patient about 125 mg of the compound of Formula (I) or pharmaceutically acceptable salt thereof three times a week. In some embodiments, the method comprises administering to the patient about 130 mg of the compound of Formula (I) or pharmaceutically acceptable salt thereof three times a week. In some embodiments, the method comprises administering to the patient about 135 mg of the compound of Formula (I) or pharmaceutically acceptable salt thereof three times a week. In some embodiments, the method comprises administering to the patient about 140 mg of the compound of Formula (I) or pharmaceutically acceptable salt thereof three times a week. In some embodiments, the method comprises administering to the patient about 145 mg of the compound of Formula (I) or pharmaceutically acceptable salt thereof three times a week. In some embodiments, the method comprises administering to the patient about 150 mg of the compound of Formula (I) or pharmaceutically acceptable salt thereof three times a week. In some embodiments, the method comprises administering to the patient about 155 mg of the compound of Formula (I) or pharmaceutically acceptable salt thereof three times a week. In some embodiments, the method comprises administering to the patient about 160 mg of the compound of Formula (I) or pharmaceutically acceptable salt thereof three times a week. In some embodiments, the method comprises administering to the patient about 165 mg of the compound of Formula (I) or pharmaceutically acceptable salt thereof three times a week. In some embodiments, the method comprises administering to the patient about 170 mg of the compound of Formula (I) or pharmaceutically acceptable salt thereof three times a week. In some embodiments, the method comprises administering to the patient about 175 mg of the compound of Formula (I) or pharmaceutically acceptable salt thereof three times a week. In some embodiments, the method comprises administering to the patient about 180 mg of the compound of Formula (I) or pharmaceutically acceptable salt thereof three times a week. In some embodiments, the method comprises administering to the patient about 185 mg of the compound of Formula (I) or pharmaceutically acceptable salt thereof three times a week. In some embodiments, the method comprises administering to the patient about 190 mg of the compound of Formula (I) or pharmaceutically acceptable salt thereof three times a week. In some embodiments, the method comprises administering to the patient about 195 mg of the compound of Formula (I) or pharmaceutically acceptable salt thereof three times a week. In some embodiments, the method comprises administering to the patient about 200 mg of the compound of Formula (I) or pharmaceutically acceptable salt thereof three times a week.

In some embodiments, the method comprises administering to the patient about 50 mg to about 200 mg of the compound of Formula (I) or pharmaceutically acceptable salt thereof every other day. In some embodiments, the method comprises administering to the patient about 50 mg to about 150 mg of the compound of Formula (I) or pharmaceutically acceptable salt thereof every other day. In some embodiments, the method comprises administering to the patient about 50 mg to about 100 mg of the compound of Formula (I) or pharmaceutically acceptable salt thereof every other day. In some embodiments, the method comprises administering to the patient about 60 mg to about 200 mg of the compound of Formula (I) or pharmaceutically acceptable salt thereof every other day. In some embodiments, the method comprises administering to the patient about 60 mg to about 150 mg of the compound of Formula (I) or pharmaceutically acceptable salt thereof every other day. In some embodiments, the method comprises administering to the patient about 60 mg to about 100 mg of the compound of Formula (I) or pharmaceutically acceptable salt thereof every other day. In some embodiments, the method comprises administering to the patient about 70 mg to about 200 mg of the compound of Formula (I) or pharmaceutically acceptable salt thereof every other day. In some embodiments, the method comprises administering to the patient about 70 mg to about 150 mg of the compound of Formula (I) or pharmaceutically acceptable salt thereof every other day. In some embodiments, the method comprises administering to the patient about 70 mg to about 100 mg of the compound of Formula (I) or pharmaceutically acceptable salt thereof every other day. In some embodiments, the method comprises administering to the patient about 80 mg to about 200 mg of the compound of Formula (I) or pharmaceutically acceptable salt thereof every other day. In some embodiments, the method comprises administering to the patient about 80 mg to about 150 mg of the compound of Formula (I) or pharmaceutically acceptable salt thereof every other day. In some embodiments, the method comprises administering to the patient about 80 mg to about 100 mg of the compound of Formula (I) or pharmaceutically acceptable salt thereof every other day. In some embodiments, the method comprises administering to the patient about 90 mg to about 200 mg of the compound of Formula (I) or pharmaceutically acceptable salt thereof every other day. In some embodiments, the method comprises administering to the patient about 90 mg to about 150 mg of the compound of Formula (I) or pharmaceutically acceptable salt thereof every other day. In some embodiments, the method comprises administering to the patient about 90 mg to about 100 mg of the compound of Formula (I) or pharmaceutically acceptable salt thereof every other day. In some embodiments, the method comprises administering to the patient about 100 mg to about 200 mg of the compound of Formula (I) or pharmaceutically acceptable salt thereof every other day. In some embodiments, the method comprises administering to the patient about 100 mg to about 150 mg of the compound of Formula (I) or pharmaceutically acceptable salt thereof every other day.

In some embodiments, the method comprises administering to the patient about 50 mg of the compound of Formula (I) or pharmaceutically acceptable salt thereof every other day. In some embodiments, the method comprises administering to the patient about 55 mg of the compound of Formula (I) or pharmaceutically acceptable salt thereof every other day. In some embodiments, the method comprises administering to the patient about 60 mg of the compound of Formula (I) or pharmaceutically acceptable salt thereof every other day. In some embodiments, the method comprises administering to the patient about 65 mg of the compound of Formula (I) or pharmaceutically acceptable salt thereof every other day. In some embodiments, the method comprises administering to the patient about 70 mg of the compound of Formula (I) or pharmaceutically acceptable salt thereof every other day. In some embodiments, the method comprises administering to the patient about 75 mg of the compound of Formula (I) or pharmaceutically acceptable salt thereof every other day. In some embodiments, the method comprises administering to the patient about 80 mg of the compound of Formula (I) or pharmaceutically acceptable salt thereof every other day. In some embodiments, the method comprises administering to the patient about 85 mg of the compound of Formula (I) or pharmaceutically acceptable salt thereof every other day. In some embodiments, the method comprises administering to the patient about 90 mg of the compound of Formula (I) or pharmaceutically acceptable salt thereof every other day. In some embodiments, the method comprises administering to the patient about 95 mg of the compound of Formula (I) or pharmaceutically acceptable salt thereof every other day. In some embodiments, the method comprises administering to the patient about 100 mg of the compound of Formula (I) or pharmaceutically acceptable salt thereof every other day. In some embodiments, the method comprises administering to the patient about 105 mg of the compound of Formula (I) or pharmaceutically acceptable salt thereof every other day. In some embodiments, the method comprises administering to the patient about 110 mg of the compound of Formula (I) or pharmaceutically acceptable salt thereof every other day. In some embodiments, the method comprises administering to the patient about 115 mg of the compound of Formula (I) or pharmaceutically acceptable salt thereof every other day. In some embodiments, the method comprises administering to the patient about 120 mg of the compound of Formula (I) or pharmaceutically acceptable salt thereof every other day. In some embodiments, the method comprises administering to the patient about 125 mg of the compound of Formula (I) or pharmaceutically acceptable salt thereof every other day. In some embodiments, the method comprises administering to the patient about 130 mg of the compound embodiments, the method comprises administering to the patient about 135 mg of the compound of Formula (I) or pharmaceutically acceptable salt thereof every other day. In some embodiments, the method comprises administering to the patient about 140 mg of the compound of Formula (I) or pharmaceutically acceptable salt thereof every other day. In some embodiments, the method comprises administering to the patient about 145 mg of the compound of Formula (I) or pharmaceutically acceptable salt thereof every other day. In some embodiments, the method comprises administering to the patient about 150 mg of the compound of Formula (I) or pharmaceutically acceptable salt thereof every other day. In some embodiments, the method comprises administering to the patient about 155 mg of the compound of Formula (I) or pharmaceutically acceptable salt thereof every other day. In some embodiments, the method comprises administering to the patient about 160 mg of the compound of Formula (I) or pharmaceutically acceptable salt thereof every other day. In some embodiments, the method comprises administering to the patient about 165 mg of the compound of Formula (I) or pharmaceutically acceptable salt thereof every other day. In some embodiments, the method comprises administering to the patient about 170 mg of the compound of Formula (I) or pharmaceutically acceptable salt thereof every other day. In some embodiments, the method comprises administering to the patient about 175 mg of the compound of Formula (I) or pharmaceutically acceptable salt thereof every other day. In some embodiments, the method comprises administering to the patient about 180 mg of the compound of Formula (I) or pharmaceutically acceptable salt thereof every other day. In some embodiments, the method comprises administering to the patient about 185 mg of the compound of Formula (I) or pharmaceutically acceptable salt thereof every other day. In some embodiments, the method comprises administering to the patient about 190 mg of the compound of Formula (I) or pharmaceutically acceptable salt thereof every other day. In some embodiments, the method comprises administering to the patient about 195 mg of the compound of Formula (I) or pharmaceutically acceptable salt thereof every other day. In some embodiments, the method comprises administering to the patient about 200 mg of the compound of Formula (I) or pharmaceutically acceptable salt thereof every other day.

In some embodiments, a method of treating a gynecologic carcinosarcoma described herein further comprises administering paclitaxel to the patient in need thereof. In one embodiment, the paclitaxel is intravenously administered to the patient, e.g., by an intravenous infusion. The intravenously administered paclitaxel can be administered to a patient at various dose levels. In some embodiments, the paclitaxel is administered to the patient at about 50 mg/m² to about 150 mg/m². In some embodiments, the paclitaxel is administered to the patient at about 50 mg/m² to about 100 mg/m². In some embodiments, the paclitaxel is administered to the patient at about 60 mg/m² to about 80 mg/m². In some embodiments, the paclitaxel is administered to the patient at about 60 mg/m². In some embodiments, the paclitaxel is administered to the patient at about 80 mg/m². In some embodiments, the paclitaxel is administered to the patient at about 135 mg/m². In some embodiments, the paclitaxel is administered to the patient at about 175 mg/m².

Furthermore, the paclitaxel can be administered to the patient at various frequencies. In some embodiments, the paclitaxel is administered to the patient once per week (e.g., at about 60 mg/m² once per week or about 80 mg/m² once per week). In some embodiments, the paclitaxel is administered to the patient once every three weeks (e.g., at about 135 mg/m² once every three weeks or about 175 mg/m² once every three weeks). In some embodiments, the paclitaxel is administered to the patient by intravenous infusion once per week. In some embodiments, the paclitaxel is administered to the patient by intravenous infusion once per week for three consecutive weeks. In some embodiments, the method comprises administering paclitaxel to the patient by intravenous infusion for more than one cycle, wherein each cycle comprises administering paclitaxel to the patient by intravenous infusion once per week for three consecutive weeks (e.g., once per week for three consecutive weeks at about 60 mg/m² or about 80 mg/m²). In some embodiments, the method comprises not administering paclitaxel to the patient for at least one week between each cycle.

In some embodiments, the paclitaxel is administered to the patient by infusion once every three weeks. In some embodiments, the paclitaxel is administered to the patient for about 60 minutes once per week. In some embodiments, the paclitaxel is administered to the patient for about 3 hours once per week. In some embodiments, the paclitaxel is administered to the patient for about 60 minutes once every three weeks. In some embodiments, the paclitaxel is administered to the patient for about 3 hours once every three weeks. In some embodiments, the paclitaxel is administered to the patient by intravenous infusion for about 60 minutes once per week. In some embodiments, the paclitaxel is administered to the patient by infusion for about 3 hours once per week. In some embodiments, the paclitaxel is administered to the patient by intravenous infusion for about 60 minutes once every three weeks. In some embodiments, the paclitaxel is administered to the patient by infusion for about 3 hours once every three weeks. In some embodiments, the paclitaxel is continuously administered to the patient for about 60 minutes once per week. In some embodiments, the paclitaxel is continuously administered to the patient for about 3 hours once per week. In some embodiments, the paclitaxel is continuously administered to the patient for about 60 minutes once every three weeks. In some embodiments, the paclitaxel is continuously administered to the patient for about 3 hours once every three weeks. In some embodiments, the paclitaxel is continuously administered to the patient by infusion for about 60 minutes once per week. In some embodiments, the paclitaxel is continuously administered to the patient by intravenous infusion for about 3 hours once per week. In some embodiments, the paclitaxel is continuously administered to the patient by infusion for about 60 minutes once every three weeks. In some embodiments, the paclitaxel is continuously administered to the patient by intravenous infusion for about 3 hours once every three weeks.

In embodiments of the methods of treating a gynecologic carcinosarcoma described herein, the disclosure also contemplates administration of the compound of Formula (I) or a pharmaceutically acceptable salt to a patient in need thereof prior to (neo-adjuvant) or after (adjuvant) surgery (e.g., surgical treatment of a solid tumor). In some embodiments, the compound of Formula (I) or a pharmaceutically acceptable salt is administered as an adjuvant. In some embodiments, the compound of Formula (I) or a pharmaceutically acceptable salt is administered as a neo-adjuvant. In some embodiments, the compound of Formula (I) or a pharmaceutically acceptable salt is administered as a neo-adjuvant and an adjuvant. In some embodiments, the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 100 years. In some embodiments, administration of the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 5 years. In some embodiments, administration of the compound of Formula (I) or a pharmaceutically acceptable salt is administered to a patient in need thereof as a neo-adjuvant for a period of 3 months to 6 months, followed by administration of the compound of Formula (I) or a pharmaceutically acceptable salt as an adjuvant for a period from 1 day to 5 years. In some embodiments, no administration of the compound of Formula (I) or a pharmaceutically acceptable salt occurs prior to surgery. In some embodiments, the compound of Formula (I) or a pharmaceutically acceptable salt is administered as an adjuvant for a period from 1 day to 100 years. In some embodiments, the compound of Formula (I) or a pharmaceutically acceptable salt is administered as an adjuvant for a period from 1 day to 5 years.

### Endometrial Adenocarcinomas

Also provided herein is a method of treating endometrial adenocarcinoma in a patient in need thereof, comprising administering to the patient an effective amount of the compound of Formula (I)

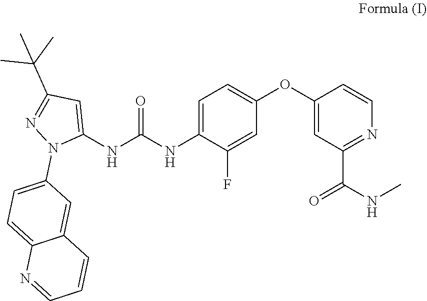 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In an embodiment, the patient has previously been administered at least one prior line of platinum-based therapy.

In some embodiments, the endometrial adenocarcinoma is characterized as having a serous histology. In some embodiments, the endometrial adenocarcinoma is characterized as having an endometroid histology. In some embodiments, the endometrial adenocarcinoma is a Grade 1 endometrioid adenocarcinoma. In some embodiments, the endometrial cancer is a Grade 2 endometrioid adenocarcinoma. In some embodiments, the endometrial cancer is a Grade 3 endometrioid adenocarcinoma.

A patient treated by the methods of treating an endometrial adenocarcinoma described herein can achieve a partial response condition or a stable disease condition. In some embodiments, after 28 days or more of administering the compound of Formula (I) or pharmaceutically acceptable salt thereof to the patient, the patient has at least a partial response or a stable disease condition.

In some embodiments, the endometrial adenocarcinoma is characterized as having a serous histology. In some embodiments, the endometrial adenocarcinoma is characterized as having an endometroid histology. In some embodiments, the endometrial adenocarcinoma is a Grade 1 endometrioid adenocarcinoma. In some embodiments, the endometrial cancer is a Grade 2 endometrioid adenocarcinoma. In some embodiments, the endometrial cancer is a Grade 3 endometrioid adenocarcinoma.

In embodiments of the methods of treating an endometrial adenocarcinoma described herein, the compound of Formula (I) or pharmaceutically acceptable salt thereof can be orally administered to the patient in need thereof. In some embodiments, the method comprises administering to the patient about 50 mg to about 200 mg of the compound of Formula (I) or pharmaceutically acceptable salt thereof per day. In certain embodiments, the compound of Formula (I) or pharmaceutically acceptable salt thereof is administered to the patient daily. In some embodiments, the method comprises administering to the patient about 50 mg to about 150 mg of the compound of Formula (I) or pharmaceutically acceptable salt thereof per day. In some embodiments, the method comprises administering to the patient about 50 mg to about 100 mg of the compound of Formula (I) or pharmaceutically acceptable salt thereof per day. In some embodiments, the method comprises administering to the patient about 60 mg to about 200 mg of the compound of Formula (I) or pharmaceutically acceptable salt thereof per day. In some embodiments, the method comprises administering to the patient about 60 mg to about 150 mg of the compound of Formula (I) or pharmaceutically acceptable salt thereof per day. In some embodiments, the method comprises administering to the patient about 60 mg to about 100 mg of the compound of Formula (I) or pharmaceutically acceptable salt thereof per day. In some embodiments, the method comprises administering to the patient about 70 mg to about 200 mg of the compound of Formula (I) or pharmaceutically acceptable salt thereof per day. In some embodiments, the method comprises administering to the patient about 70 mg to about 150 mg of the compound of Formula (I) or pharmaceutically acceptable salt thereof per day. In some embodiments, the method comprises administering to the patient about 70 mg to about 100 mg of the compound of Formula (I) or pharmaceutically acceptable salt thereof per day. In some embodiments, the method comprises administering to the patient about 80 mg to about 200 mg of the compound of Formula (I) or pharmaceutically acceptable salt thereof per day. In some embodiments, the method comprises administering to the patient about 80 mg to about 150 mg of the compound of Formula (I) or pharmaceutically acceptable salt thereof per day. In some embodiments, the method comprises administering to the patient about 80 mg to about 100 mg of the compound of Formula (I) or pharmaceutically acceptable salt thereof per day. In some embodiments, the method comprises administering to the patient about 90 mg to about 200 mg of the compound of Formula (I) or pharmaceutically acceptable salt thereof per day. In some embodiments, the method comprises administering to the patient about 90 mg to about 150 mg of the compound of Formula (I) or pharmaceutically acceptable salt thereof per day. In some embodiments, the method comprises administering to the patient about 90 mg to about 100 mg of the compound of Formula (I) or pharmaceutically acceptable salt thereof per day. In some embodiments, the method comprises administering to the patient about 100 mg to about 200 mg of the compound of Formula (I) or pharmaceutically acceptable salt thereof per day. In some embodiments, the method comprises administering to the patient about 100 mg to about 150 mg of the compound of Formula (I) or pharmaceutically acceptable salt thereof per day.

In some embodiments, the method comprises administering to the patient about 50 mg of the compound of Formula (I) or pharmaceutically acceptable salt thereof per day. In some embodiments, the method comprises administering to the patient about 55 mg of the compound of Formula (I) or pharmaceutically acceptable salt thereof per day. In some embodiments, the method comprises administering to the patient about 60 mg of the compound of Formula (I) or pharmaceutically acceptable salt thereof per day. In some embodiments, the method comprises administering to the patient about 65 mg of the compound of Formula (I) or pharmaceutically acceptable salt thereof per day. In some embodiments, the method comprises administering to the patient about 70 mg of the compound of Formula (I) or pharmaceutically acceptable salt thereof per day. In some embodiments, the method comprises administering to the patient about 75 mg of the compound of Formula (I) or pharmaceutically acceptable salt thereof per day. In some embodiments, the method comprises administering to the patient about 80 mg of the compound of Formula (I) or pharmaceutically acceptable salt thereof per day. In some embodiments, the method comprises administering to the patient about 85 mg of the compound of Formula (I) or pharmaceutically acceptable salt thereof per day. In some embodiments, the method comprises administering to the patient about 90 mg of the compound of Formula (I) or pharmaceutically acceptable salt thereof per day. In some embodiments, the method comprises administering to the patient about 95 mg of the compound of Formula (I) or pharmaceutically acceptable salt thereof per day. In some embodiments, the method comprises administering to the patient about 100 mg of the compound of Formula (I) or pharmaceutically acceptable salt thereof per day. In some embodiments, the method comprises administering to the patient about 105 mg of the compound of Formula (I) or pharmaceutically acceptable salt thereof per day. In some embodiments, the method comprises administering to the patient about 110 mg of the compound of Formula (I) or pharmaceutically acceptable salt thereof per day. In some embodiments, the method comprises administering to the patient about 115 mg of the compound of Formula (I) or pharmaceutically acceptable salt thereof per day. In some embodiments, the method comprises administering to the patient about 120 mg of the compound of Formula (I) or pharmaceutically acceptable salt thereof per day. In some embodiments, the method comprises administering to the patient about 125 mg of the compound of Formula (I) or pharmaceutically acceptable salt thereof per day. In some embodiments, the method comprises administering to the patient about 130 mg of the compound of Formula (I) or pharmaceutically acceptable salt thereof per day. In some embodiments, the method comprises administering to the patient about 135 mg of the compound of Formula (I) or pharmaceutically acceptable salt thereof per day. In some embodiments, the method comprises administering to the patient about 140 mg of the compound of Formula (I) or pharmaceutically acceptable salt thereof per day. In some embodiments, the method comprises administering to the patient about 145 mg of the compound of Formula (I) or pharmaceutically acceptable salt thereof per day. In some embodiments, the method comprises administering to the patient about 150 mg of the compound of Formula (I) or pharmaceutically acceptable salt thereof per day. In some embodiments, the method comprises administering to the patient about 155 mg of the compound of Formula (I) or pharmaceutically acceptable salt thereof per day. In some embodiments, the method comprises administering to the patient about 160 mg of the compound of Formula (I) or pharmaceutically acceptable salt thereof per day. In some embodiments, the method comprises administering to the patient about 165 mg of the compound of Formula (I) or pharmaceutically acceptable salt thereof per day. In some embodiments, the method comprises administering to the patient about 170 mg of the compound of Formula (I) or pharmaceutically acceptable salt thereof per day. In some embodiments, the method comprises administering to the patient about 175 mg of the compound of Formula (I) or pharmaceutically acceptable salt thereof per day. In some embodiments, the method comprises administering to the patient about 180 mg of the compound of Formula (I) or pharmaceutically acceptable salt thereof per day. In some embodiments, the method comprises administering to the patient about 185 mg of the compound of Formula (I) or pharmaceutically acceptable salt thereof per day. In some embodiments, the method comprises administering to the patient about 190 mg of the compound of Formula (I) or pharmaceutically acceptable salt thereof per day. In some embodiments, the method comprises administering to the patient about 195 mg of the compound of Formula (I) or pharmaceutically acceptable salt thereof per day. In some embodiments, the method comprises administering to the patient about 200 mg of the compound of Formula (I) or pharmaceutically acceptable salt thereof per day.

In some embodiments, the method comprises administering to the patient about 50 mg to about 200 mg of the compound of Formula (I) or pharmaceutically acceptable salt thereof once per day. In some embodiments, the method comprises administering to the patient about 50 mg to about 150 mg of the compound of Formula (I) or pharmaceutically acceptable salt thereof once per day. In some embodiments, the method comprises administering to the patient about 50 mg to about 100 mg of the compound of Formula (I) or pharmaceutically acceptable salt thereof once per day. In some embodiments, the method comprises administering to the patient about 60 mg to about 200 mg of the compound of Formula (I) or pharmaceutically acceptable salt thereof once per day. In some embodiments, the method comprises administering to the patient about 60 mg to about 150 mg of the compound of Formula (I) or pharmaceutically acceptable salt thereof once per day. In some embodiments, the method comprises administering to the patient about 60 mg to about 100 mg of the compound of Formula (I) or pharmaceutically acceptable salt thereof once per day. In some embodiments, the method comprises administering to the patient about 70 mg to about 200 mg of the compound of Formula (I) or pharmaceutically acceptable salt thereof once per day. In some embodiments, the method comprises administering to the patient about 70 mg to about 150 mg of the compound of Formula (I) or pharmaceutically acceptable salt thereof once per day. In some embodiments, the method comprises administering to the patient about 70 mg to about 100 mg of the compound of Formula (I) or pharmaceutically acceptable salt thereof once per day. In some embodiments, the method comprises administering to the patient about 80 mg to about 200 mg of the compound of Formula (I) or pharmaceutically acceptable salt thereof once per day. In some embodiments, the method comprises administering to the patient about 80 mg to about 150 mg of the compound of Formula (I) or pharmaceutically acceptable salt thereof once per day. In some embodiments, the method comprises administering to the patient about 80 mg to about 100 mg of the compound of Formula (I) or pharmaceutically acceptable salt thereof once per day. In some embodiments, the method comprises administering to the patient about 90 mg to about 200 mg of the compound of Formula (I) or pharmaceutically acceptable salt thereof once per day. In some embodiments, the method comprises administering to the patient about 90 mg to about 150 mg of the compound of Formula (I) or pharmaceutically acceptable salt thereof once per day. In some embodiments, the method comprises administering to the patient about 90 mg to about 100 mg of the compound of Formula (I) or pharmaceutically acceptable salt thereof once per day. In some embodiments, the method comprises administering to the patient about 100 mg to about 200 mg of the compound of Formula (I) or pharmaceutically acceptable salt thereof once per day. In some embodiments, the method comprises administering to the patient about 100 mg to about 150 mg of the compound of Formula (I) or pharmaceutically acceptable salt thereof once per day.

In some embodiments, the method comprises administering to the patient about 50 mg of the compound of Formula (I) or pharmaceutically acceptable salt thereof once per day. In some embodiments, the method comprises administering to the patient about 55 mg of the compound of Formula (I) or pharmaceutically acceptable salt thereof once per day. In some embodiments, the method comprises administering to the patient about 60 mg of the compound of Formula (I) or pharmaceutically acceptable salt thereof once per day. In some embodiments, the method comprises administering to the patient about 65 mg of the compound of Formula (I) or pharmaceutically acceptable salt thereof once per day. In some embodiments, the method comprises administering to the patient about 70 mg of the compound of Formula (I) or pharmaceutically acceptable salt thereof once per day. In some embodiments, the method comprises administering to the patient about 75 mg of the compound of Formula (I) or pharmaceutically acceptable salt thereof once per day. In some embodiments, the method comprises administering to the patient about 80 mg of the compound of Formula (I) or pharmaceutically acceptable salt thereof once per day. In some embodiments, the method comprises administering to the patient about 85 mg of the compound of Formula (I) or pharmaceutically acceptable salt thereof once per day. In some embodiments, the method comprises administering to the patient about 90 mg of the compound of Formula (I) or pharmaceutically acceptable salt thereof once per day. In some embodiments, the method comprises administering to the patient about 95 mg of the compound of Formula (I) or pharmaceutically acceptable salt thereof once per day. In some embodiments, the method comprises administering to the patient about 100 mg of the compound of Formula (I) or pharmaceutically acceptable salt thereof once per day. In some embodiments, the method comprises administering to the patient about 105 mg of the compound of Formula (I) or pharmaceutically acceptable salt thereof once per day. In some embodiments, the method comprises administering to the patient about 110 mg of the compound of Formula (I) or pharmaceutically acceptable salt thereof once per day. In some embodiments, the method comprises administering to the patient about 115 mg of the compound of Formula (I) or pharmaceutically acceptable salt thereof once per day. In some embodiments, the method comprises administering to the patient about 120 mg of the compound of Formula (I) or pharmaceutically acceptable salt thereof once per day. In some embodiments, the method comprises administering to the patient about 125 mg of the compound of Formula (I) or pharmaceutically acceptable salt thereof once per day. In some embodiments, the method comprises administering to the patient about 130 mg of the compound of Formula (I) or pharmaceutically acceptable salt thereof once per day. In some embodiments, the method comprises administering to the patient about 135 mg of the compound of Formula (I) or pharmaceutically acceptable salt thereof once per day. In some embodiments, the method comprises administering to the patient about 140 mg of the compound of Formula (I) or pharmaceutically acceptable salt thereof once per day. In some embodiments, the method comprises administering to the patient about 145 mg of the compound of Formula (I) or pharmaceutically acceptable salt thereof once per day. In some embodiments, the method comprises administering to the patient about 150 mg of the compound of Formula (I) or pharmaceutically acceptable salt thereof once per day. In some embodiments, the method comprises administering to the patient about 155 mg of the compound of Formula (I) or pharmaceutically acceptable salt thereof once per day. In some embodiments, the method comprises administering to the patient about 160 mg of the compound of Formula (I) or pharmaceutically acceptable salt thereof once per day. In some embodiments, the method comprises administering to the patient about 165 mg of the compound of Formula (I) or pharmaceutically acceptable salt thereof once per day. In some embodiments, the method comprises administering to the patient about 170 mg of the compound of Formula (I) or pharmaceutically acceptable salt thereof once per day. In some embodiments, the method comprises administering to the patient about 175 mg of the compound of Formula (I) or pharmaceutically acceptable salt thereof once per day. In some embodiments, the method comprises administering to the patient about 180 mg of the compound of Formula (I) or pharmaceutically acceptable salt thereof once per day. In some embodiments, the method comprises administering to the patient about 185 mg of the compound of Formula (I) or pharmaceutically acceptable salt thereof once per day. In some embodiments, the method comprises administering to the patient about 190 mg of the compound of Formula (I) or pharmaceutically acceptable salt thereof once per day. In some embodiments, the method comprises administering to the patient about 195 mg of the compound of Formula (I) or pharmaceutically acceptable salt thereof once per day. In some embodiments, the method comprises administering to the patient about 200 mg of the compound of Formula (I) or pharmaceutically acceptable salt thereof once per day.

In some embodiments, the method comprises administering to the patient about 50 mg to about 100 mg of the compound of Formula (I) or pharmaceutically acceptable salt thereof twice per day. In some embodiments, the method comprises administering to the patient about 60 mg to about 100 mg of the compound of Formula (I) or pharmaceutically acceptable salt thereof twice per day. In some embodiments, the method comprises administering to the patient about 70 mg to about 100 mg of the compound of Formula (I) or pharmaceutically acceptable salt thereof twice per day. In some embodiments, the method comprises administering to the patient about 80 mg to about 100 mg of the compound of Formula (I) or pharmaceutically acceptable salt thereof twice per day. In some embodiments, the method comprises administering to the patient about 90 mg to about 100 mg of the compound of Formula (I) or pharmaceutically acceptable salt thereof twice per day.

In some embodiments, the method comprises administering to the patient about 50 mg of the compound of Formula (I) or pharmaceutically acceptable salt thereof twice per day. In some embodiments, the method comprises administering to the patient about 55 mg of the compound of Formula (I) or pharmaceutically acceptable salt thereof twice per day. In some embodiments, the method comprises administering to the patient about 60 mg of the compound of Formula (I) or pharmaceutically acceptable salt thereof twice per day. In some embodiments, the method comprises administering to the patient about 65 mg of the compound of Formula (I) or pharmaceutically acceptable salt thereof twice per day. In some embodiments, the method comprises administering to the patient about 70 mg of the compound of Formula (I) or pharmaceutically acceptable salt thereof twice per day. In some embodiments, the method comprises administering to the patient about 75 mg of the compound of Formula (I) or pharmaceutically acceptable salt thereof twice per day. In some embodiments, the method comprises administering to the patient about 80 mg of the compound of Formula (I) or pharmaceutically acceptable salt thereof twice per day. In some embodiments, the method comprises administering to the patient about 85 mg of the compound of Formula (I) or pharmaceutically acceptable salt thereof twice per day. In some embodiments, the method comprises administering to the patient about 90 mg of the compound of Formula (I) or pharmaceutically acceptable salt thereof twice per day. In some embodiments, the method comprises administering to the patient about 95 mg of the compound of Formula (I) or pharmaceutically acceptable salt thereof twice per day. In some embodiments, the method comprises administering to the patient about 100 mg of the compound of Formula (I) or pharmaceutically acceptable salt thereof twice per day.

The compound of Formula (I) or pharmaceutically acceptable salt thereof may, in another embodiment, be administered to the patient on an intermittent basis in the methods of treating endometrial adenocarcinoma described herein. In some embodiments, the compound of Formula (I) or pharmaceutically acceptable salt thereof is administered the patient every other day. In some embodiments, the compound of Formula (I) or pharmaceutically acceptable salt thereof is administered the patient every other day. In some embodiments, the compound of Formula (I) or pharmaceutically acceptable salt thereof is administered the patient once per week. In some embodiments, the compound of Formula (I) or pharmaceutically acceptable salt thereof is administered the patient twice per week. In some embodiments, the compound of Formula (I) or pharmaceutically acceptable salt thereof is administered the patient three times per week.

In some embodiments, the method comprises administering to the patient about 50 mg to about 200 mg of the compound of Formula (I) or pharmaceutically acceptable salt thereof once a week. In some embodiments, the method comprises administering to the patient about 50 mg to about 150 mg of the compound of Formula (I) or pharmaceutically acceptable salt thereof once a week. In some embodiments, the method comprises administering to the patient about 50 mg to about 100 mg of the compound of Formula (I) or pharmaceutically acceptable salt thereof once a week. In some embodiments, the method comprises administering to the patient about 60 mg to about 200 mg of the compound of Formula (I) or pharmaceutically acceptable salt thereof once a week. In some embodiments, the method comprises administering to the patient about 60 mg to about 150 mg of the compound of Formula (I) or pharmaceutically acceptable salt thereof once a week. In some embodiments, the method comprises administering to the patient about 60 mg to about 100 mg of the compound of Formula (I) or pharmaceutically acceptable salt thereof once a week. In some embodiments, the method comprises administering to the patient about 70 mg to about 200 mg of the compound of Formula (I) or pharmaceutically acceptable salt thereof once a week. In some embodiments, the method comprises administering to the patient about 70 mg to about 150 mg of the compound of Formula (I) or pharmaceutically acceptable salt thereof once a week. In some embodiments, the method comprises administering to the patient about 70 mg to about 100 mg of the compound of Formula (I) or pharmaceutically acceptable salt thereof once a week. In some embodiments, the method comprises administering to the patient about 80 mg to about 200 mg of the compound of Formula (I) or pharmaceutically acceptable salt thereof once a week. In some embodiments, the method comprises administering to the patient about 80 mg to about 150 mg of the compound of Formula (I) or pharmaceutically acceptable salt thereof once a week. In some embodiments, the method comprises administering to the patient about 80 mg to about 100 mg of the compound of Formula (I) or pharmaceutically acceptable salt thereof once a week. In some embodiments, the method comprises administering to the patient about 90 mg to about 200 mg of the compound of Formula (I) or pharmaceutically acceptable salt thereof once a week. In some embodiments, the method comprises administering to the patient about 90 mg to about 150 mg of the compound of Formula (I) or pharmaceutically acceptable salt thereof once a week. In some embodiments, the method comprises administering to the patient about 90 mg to about 100 mg of the compound of Formula (I) or pharmaceutically acceptable salt thereof once a week. In some embodiments, the method comprises administering to the patient about 100 mg to about 200 mg of the compound of Formula (I) or pharmaceutically acceptable salt thereof once a week. In some embodiments, the method comprises administering to the patient about 100 mg to about 150 mg of the compound of Formula (I) or pharmaceutically acceptable salt thereof once a week.

In some embodiments, the method comprises administering to the patient about 50 mg of the compound of Formula (I) or pharmaceutically acceptable salt thereof once a week. In some embodiments, the method comprises administering to the patient about 55 mg of the compound of Formula (I) or pharmaceutically acceptable salt thereof once a week. In some embodiments, the method comprises administering to the patient about 60 mg of the compound of Formula (I) or pharmaceutically acceptable salt thereof once a week. In some embodiments, the method comprises administering to the patient about 65 mg of the compound of Formula (I) or pharmaceutically acceptable salt thereof once a week. In some embodiments, the method comprises administering to the patient about 70 mg of the compound of Formula (I) or pharmaceutically acceptable salt thereof once a week. In some embodiments, the method comprises administering to the patient about 75 mg of the compound of Formula (I) or pharmaceutically acceptable salt thereof once a week. In some embodiments, the method comprises administering to the patient about 80 mg of the compound of Formula (I) or pharmaceutically acceptable salt thereof once a week. In some embodiments, the method comprises administering to the patient about 85 mg of the compound of Formula (I) or pharmaceutically acceptable salt thereof once a week. In some embodiments, the method comprises administering to the patient about 90 mg of the compound of Formula (I) or pharmaceutically acceptable salt thereof once a week. In some embodiments, the method comprises administering to the patient about 95 mg of the compound of Formula (I) or pharmaceutically acceptable salt thereof once a week. In some embodiments, the method comprises administering to the patient about 100 mg of the compound of Formula (I) or pharmaceutically acceptable salt thereof once a week. In some embodiments, the method comprises administering to the patient about 105 mg of the compound of Formula (I) or pharmaceutically acceptable salt thereof once a week. In some embodiments, the method comprises administering to the patient about 110 mg of the compound of Formula (I) or pharmaceutically acceptable salt thereof once a week. In some embodiments, the method comprises administering to the patient about 115 mg of the compound of Formula (I) or pharmaceutically acceptable salt thereof once a week. In some embodiments, the method comprises administering to the patient about 120 mg of the compound of Formula (I) or pharmaceutically acceptable salt thereof once a week. In some embodiments, the method comprises administering to the patient about 125 mg of the compound of Formula (I) or pharmaceutically acceptable salt thereof once a week. In some embodiments, the method comprises administering to the patient about 130 mg of the compound of Formula (I) or pharmaceutically acceptable salt thereof once a week. In some embodiments, the method comprises administering to the patient about 135 mg of the compound of Formula (I) or pharmaceutically acceptable salt thereof once a week. In some embodiments, the method comprises administering to the patient about 140 mg of the compound of Formula (I) or pharmaceutically acceptable salt thereof once a week. In some embodiments, the method comprises administering to the patient about 145 mg of the compound of Formula (I) or pharmaceutically acceptable salt thereof once a week. In some embodiments, the method comprises administering to the patient about 150 mg of the compound of Formula (I) or pharmaceutically acceptable salt thereof once a week. In some embodiments, the method comprises administering to the patient about 155 mg of the compound of Formula (I) or pharmaceutically acceptable salt thereof once a week. In some embodiments, the method comprises administering to the patient about 160 mg of the compound of Formula (I) or pharmaceutically acceptable salt thereof once a week. In some embodiments, the method comprises administering to the patient about 165 mg of the compound of Formula (I) or pharmaceutically acceptable salt thereof once a week. In some embodiments, the method comprises administering to the patient about 170 mg of the compound of Formula (I) or pharmaceutically acceptable salt thereof once a week. In some embodiments, the method comprises administering to the patient about 175 mg of the compound of Formula (I) or pharmaceutically acceptable salt thereof once a week. In some embodiments, the method comprises administering to the patient about 180 mg of the compound of Formula (I) or pharmaceutically acceptable salt thereof once a week. In some embodiments, the method comprises administering to the patient about 185 mg of the compound of Formula (I) or pharmaceutically acceptable salt thereof once a week. In some embodiments, the method comprises administering to the patient about 190 mg of the compound of Formula (I) or pharmaceutically acceptable salt thereof once a week. In some embodiments, the method comprises administering to the patient about 195 mg of the compound of Formula (I) or pharmaceutically acceptable salt thereof once a week. In some embodiments, the method comprises administering to the patient about 200 mg of the compound of Formula (I) or pharmaceutically acceptable salt thereof once a week.

In some embodiments, the method comprises administering to the patient about 50 mg to about 200 mg of the compound of Formula (I) or pharmaceutically acceptable salt thereof twice a week. In some embodiments, the method comprises administering to the patient about 50 mg to about 150 mg of the compound of Formula (I) or pharmaceutically acceptable salt thereof twice a week. In some embodiments, the method comprises administering to the patient about 50 mg to about 100 mg of the compound of Formula (I) or pharmaceutically acceptable salt thereof twice a week. In some embodiments, the method comprises administering to the patient about 60 mg to about 200 mg of the compound of Formula (I) or pharmaceutically acceptable salt thereof twice a week. In some embodiments, the method comprises administering to the patient about 60 mg to about 150 mg of the compound of Formula (I) or pharmaceutically acceptable salt thereof twice a week. In some embodiments, the method comprises administering to the patient about 60 mg to about 100 mg of the compound of Formula (I) or pharmaceutically acceptable salt thereof twice a week. In some embodiments, the method comprises administering to the patient about 70 mg to about 200 mg of the compound of Formula (I) or pharmaceutically acceptable salt thereof twice a week. In some embodiments, the method comprises administering to the patient about 70 mg to about 150 mg of the compound of Formula (I) or pharmaceutically acceptable salt thereof twice a week. In some embodiments, the method comprises administering to the patient about 70 mg to about 100 mg of the compound of Formula (I) or pharmaceutically acceptable salt thereof twice a week. In some embodiments, the method comprises administering to the patient about 80 mg to about 200 mg of the compound of Formula (I) or pharmaceutically acceptable salt thereof twice a week. In some embodiments, the method comprises administering to the patient about 80 mg to about 150 mg of the compound of Formula (I) or pharmaceutically acceptable salt thereof twice a week. In some embodiments, the method comprises administering to the patient about 80 mg to about 100 mg of the compound of Formula (I) or pharmaceutically acceptable salt thereof twice a week. In some embodiments, the method comprises administering to the patient about 90 mg to about 200 mg of the compound of Formula (I) or pharmaceutically acceptable salt thereof twice a week. In some embodiments, the method comprises administering to the patient about 90 mg to about 150 mg of the compound of Formula (I) or pharmaceutically acceptable salt thereof twice a week. In some embodiments, the method comprises administering to the patient about 90 mg to about 100 mg of the compound of Formula (I) or pharmaceutically acceptable salt thereof twice a week. In some embodiments, the method comprises administering to the patient about 100 mg to about 200 mg of the compound of Formula (I) or pharmaceutically acceptable salt thereof twice a week. In some embodiments, the method comprises administering to the patient about 100 mg to about 150 mg of the compound of Formula (I) or pharmaceutically acceptable salt thereof twice a week.

In some embodiments, the method comprises administering to the patient about 50 mg of the compound of Formula (I) or pharmaceutically acceptable salt thereof twice a week. In some embodiments, the method comprises administering to the patient about 55 mg of the compound of Formula (I) or pharmaceutically acceptable salt thereof twice a week. In some embodiments, the method comprises administering to the patient about 60 mg of the compound of Formula (I) or pharmaceutically acceptable salt thereof twice a week. In some embodiments, the method comprises administering to the patient about 65 mg of the compound of Formula (I) or pharmaceutically acceptable salt thereof twice a week. In some embodiments, the method comprises administering to the patient about 70 mg of the compound of Formula (I) or pharmaceutically acceptable salt thereof twice a week. In some embodiments, the method comprises administering to the patient about 75 mg of the compound of Formula (I) or pharmaceutically acceptable salt thereof twice a week. In some embodiments, the method comprises administering to the patient about 80 mg of the compound of Formula (I) or pharmaceutically acceptable salt thereof twice a week. In some embodiments, the method comprises administering to the patient about 85 mg of the compound of Formula (I) or pharmaceutically acceptable salt thereof twice a week. In some embodiments, the method comprises administering to the patient about 90 mg of the compound of Formula (I) or pharmaceutically acceptable salt thereof twice a week. In some embodiments, the method comprises administering to the patient about 95 mg of the compound of Formula (I) or pharmaceutically acceptable salt thereof twice a week. In some embodiments, the method comprises administering to the patient about 100 mg of the compound of Formula (I) or pharmaceutically acceptable salt thereof twice a week. In some embodiments, the method comprises administering to the patient about 105 mg of the compound of Formula (I) or pharmaceutically acceptable salt thereof twice a week. In some embodiments, the method comprises administering to the patient about 110 mg of the compound of Formula (I) or pharmaceutically acceptable salt thereof twice a week. In some embodiments, the method comprises administering to the patient about 115 mg of the compound of Formula (I) or pharmaceutically acceptable salt thereof twice a week. In some embodiments, the method comprises administering to the patient about 120 mg of the compound of Formula (I) or pharmaceutically acceptable salt thereof twice a week. In some embodiments, the method comprises administering to the patient about 125 mg of the compound of Formula (I) or pharmaceutically acceptable salt thereof twice a week. In some embodiments, the method comprises administering to the patient about 130 mg of the compound of Formula (I) or pharmaceutically acceptable salt thereof twice a week. In some embodiments, the method comprises administering to the patient about 135 mg of the compound of Formula (I) or pharmaceutically acceptable salt thereof twice a week. In some embodiments, the method comprises administering to the patient about 140 mg of the compound of Formula (I) or pharmaceutically acceptable salt thereof twice a week. In some embodiments, the method comprises administering to the patient about 145 mg of the compound of Formula (I) or pharmaceutically acceptable salt thereof twice a week. In some embodiments, the method comprises administering to the patient about 150 mg of the compound of Formula (I) or pharmaceutically acceptable salt thereof twice a week. In some embodiments, the method comprises administering to the patient about 155 mg of the compound of Formula (I) or pharmaceutically acceptable salt thereof twice a week. In some embodiments, the method comprises administering to the patient about 160 mg of the compound of Formula (I) or pharmaceutically acceptable salt thereof twice a week. In some embodiments, the method comprises administering to the patient about 165 mg of the compound of Formula (I) or pharmaceutically acceptable salt thereof twice a week. In some embodiments, the method comprises administering to the patient about 170 mg of the compound of Formula (I) or pharmaceutically acceptable salt thereof twice a week. In some embodiments, the method comprises administering to the patient about 175 mg of the compound of Formula (I) or pharmaceutically acceptable salt thereof twice a week. In some embodiments, the method comprises administering to the patient about 180 mg of the compound of Formula (I) or pharmaceutically acceptable salt thereof twice a week. In some embodiments, the method comprises administering to the patient about 185 mg of the compound of Formula (I) or pharmaceutically acceptable salt thereof twice a week. In some embodiments, the method comprises administering to the patient about 190 mg of the compound of Formula (I) or pharmaceutically acceptable salt thereof twice a week. In some embodiments, the method comprises administering to the patient about 195 mg of the compound of Formula (I) or pharmaceutically acceptable salt thereof twice a week. In some embodiments, the method comprises administering to the patient about 200 mg of the compound of Formula (I) or pharmaceutically acceptable salt thereof twice a week.

In some embodiments, the method comprises administering to the patient about 50 mg to about 200 mg of the compound of Formula (I) or pharmaceutically acceptable salt thereof three times a week. In some embodiments, the method comprises administering to the patient about 50 mg to about 150 mg of the compound of Formula (I) or pharmaceutically acceptable salt thereof three times a week. In some embodiments, the method comprises administering to the patient about 50 mg to about 100 mg of the compound of Formula (I) or pharmaceutically acceptable salt thereof three times a week. In some embodiments, the method comprises administering to the patient about 60 mg to about 200 mg of the compound of Formula (I) or pharmaceutically acceptable salt thereof three times a week. In some embodiments, the method comprises administering to the patient about 60 mg to about 150 mg of the compound of Formula (I) or pharmaceutically acceptable salt thereof three times a week. In some embodiments, the method comprises administering to the patient about 60 mg to about 100 mg of the compound of Formula (I) or pharmaceutically acceptable salt thereof three times a week. In some embodiments, the method comprises administering to the patient about 70 mg to about 200 mg of the compound of Formula (I) or pharmaceutically acceptable salt thereof three times a week. In some embodiments, the method comprises administering to the patient about 70 mg to about 150 mg of the compound of Formula (I) or pharmaceutically acceptable salt thereof three times a week. In some embodiments, the method comprises administering to the patient about 70 mg to about 100 mg of the compound of Formula (I) or pharmaceutically acceptable salt thereof three times a week. In some embodiments, the method comprises administering to the patient about 80 mg to about 200 mg of the compound of Formula (I) or pharmaceutically acceptable salt thereof three times a week. In some embodiments, the method comprises administering to the patient about 80 mg to about 150 mg of the compound of Formula (I) or pharmaceutically acceptable salt thereof three times a week. In some embodiments, the method comprises administering to the patient about 80 mg to about 100 mg of the compound of Formula (I) or pharmaceutically acceptable salt thereof three times a week. In some embodiments, the method comprises administering to the patient about 90 mg to about 200 mg of the compound of Formula (I) or pharmaceutically acceptable salt thereof three times a week. In some embodiments, the method comprises administering to the patient about 90 mg to about 150 mg of the compound of Formula (I) or pharmaceutically acceptable salt thereof three times a week. In some embodiments, the method comprises administering to the patient about 90 mg to about 100 mg of the compound of Formula (I) or pharmaceutically acceptable salt thereof three times a week. In some embodiments, the method comprises administering to the patient about 100 mg to about 200 mg of the compound of Formula (I) or pharmaceutically acceptable salt thereof three times a week. In some embodiments, the method comprises administering to the patient about 100 mg to about 150 mg of the compound of Formula (I) or pharmaceutically acceptable salt thereof three times a week.

In some embodiments, the method comprises administering to the patient about 50 mg of the compound of Formula (I) or pharmaceutically acceptable salt thereof three times a week. In some embodiments, the method comprises administering to the patient about 55 mg of the compound of Formula (I) or pharmaceutically acceptable salt thereof three times a week. In some embodiments, the method comprises administering to the patient about 60 mg of the compound of Formula (I) or pharmaceutically acceptable salt thereof three times a week. In some embodiments, the method comprises administering to the patient about 65 mg of the compound of Formula (I) or pharmaceutically acceptable salt thereof three times a week. In some embodiments, the method comprises administering to the patient about 70 mg of the compound of Formula (I) or pharmaceutically acceptable salt thereof three times a week. In some embodiments, the method comprises administering to the patient about 75 mg of the compound of Formula (I) or pharmaceutically acceptable salt thereof three times a week. In some embodiments, the method comprises administering to the patient about 80 mg of the compound of Formula (I) or pharmaceutically acceptable salt thereof three times a week. In some embodiments, the method comprises administering to the patient about 85 mg of the compound of Formula (I) or pharmaceutically acceptable salt thereof three times a week. In some embodiments, the method comprises administering to the patient about 90 mg of the compound of Formula (I) or pharmaceutically acceptable salt thereof three times a week. In some embodiments, the method comprises administering to the patient about 95 mg of the compound of Formula (I) or pharmaceutically acceptable salt thereof three times a week. In some embodiments, the method comprises administering to the patient about 100 mg of the compound of Formula (I) or pharmaceutically acceptable salt thereof three times a week. In some embodiments, the method comprises administering to the patient about 105 mg of the compound of Formula (I) or pharmaceutically acceptable salt thereof three times a week. In some embodiments, the method comprises administering to the patient about 110 mg of the compound of Formula (I) or pharmaceutically acceptable salt thereof three times a week. In some embodiments, the method comprises administering to the patient about 115 mg of the compound of Formula (I) or pharmaceutically acceptable salt thereof three times a week. In some embodiments, the method comprises administering to the patient about 120 mg of the compound of Formula (I) or pharmaceutically acceptable salt thereof three times a week. In some embodiments, the method comprises administering to the patient about 125 mg of the compound of Formula (I) or pharmaceutically acceptable salt thereof three times a week. In some embodiments, the method comprises administering to the patient about 130 mg of the compound of Formula (I) or pharmaceutically acceptable salt thereof three times a week. In some embodiments, the method comprises administering to the patient about 135 mg of the compound of Formula (I) or pharmaceutically acceptable salt thereof three times a week. In some embodiments, the method comprises administering to the patient about 140 mg of the compound of Formula (I) or pharmaceutically acceptable salt thereof three times a week. In some embodiments, the method comprises administering to the patient about 145 mg of the compound of Formula (I) or pharmaceutically acceptable salt thereof three times a week. In some embodiments, the method comprises administering to the patient about 150 mg of the compound of Formula (I) or pharmaceutically acceptable salt thereof three times a week. In some embodiments, the method comprises administering to the patient about 155 mg of the compound of Formula (I) or pharmaceutically acceptable salt thereof three times a week. In some embodiments, the method comprises administering to the patient about 160 mg of the compound of Formula (I) or pharmaceutically acceptable salt thereof three times a week. In some embodiments, the method comprises administering to the patient about 165 mg of the compound of Formula (I) or pharmaceutically acceptable salt thereof three times a week. In some embodiments, the method comprises administering to the patient about 170 mg of the compound of Formula (I) or pharmaceutically acceptable salt thereof three times a week. In some embodiments, the method comprises administering to the patient about 175 mg of the compound of Formula (I) or pharmaceutically acceptable salt thereof three times a week. In some embodiments, the method comprises administering to the patient about 180 mg of the compound of Formula (I) or pharmaceutically acceptable salt thereof three times a week. In some embodiments, the method comprises administering to the patient about 185 mg of the compound of Formula (I) or pharmaceutically acceptable salt thereof three times a week. In some embodiments, the method comprises administering to the patient about 190 mg of the compound of Formula (I) or pharmaceutically acceptable salt thereof three times a week. In some embodiments, the method comprises administering to the patient about 195 mg of the compound of Formula (I) or pharmaceutically acceptable salt thereof three times a week. In some embodiments, the method comprises administering to the patient about 200 mg of the compound of Formula (I) or pharmaceutically acceptable salt thereof three times a week.

In some embodiments, the method comprises administering to the patient about 50 mg to about 200 mg of the compound of Formula (I) or pharmaceutically acceptable salt thereof every other day. In some embodiments, the method comprises administering to the patient about 50 mg to about 150 mg of the compound of Formula (I) or pharmaceutically acceptable salt thereof every other day. In some embodiments, the method comprises administering to the patient about 50 mg to about 100 mg of the compound of Formula (I) or pharmaceutically acceptable salt thereof every other day. In some embodiments, the method comprises administering to the patient about 60 mg to about 200 mg of the compound of Formula (I) or pharmaceutically acceptable salt thereof every other day. In some embodiments, the method comprises administering to the patient about 60 mg to about 150 mg of the compound of Formula (I) or pharmaceutically acceptable salt thereof every other day. In some embodiments, the method comprises administering to the patient about 60 mg to about 100 mg of the compound of Formula (I) or pharmaceutically acceptable salt thereof every other day. In some embodiments, the method comprises administering to the patient about 70 mg to about 200 mg of the compound of Formula (I) or pharmaceutically acceptable salt thereof every other day. In some embodiments, the method comprises administering to the patient about 70 mg to about 150 mg of the compound of Formula (I) or pharmaceutically acceptable salt thereof every other day. In some embodiments, the method comprises administering to the patient about 70 mg to about 100 mg of the compound of Formula (I) or pharmaceutically acceptable salt thereof every other day. In some embodiments, the method comprises administering to the patient about 80 mg to about 200 mg of the compound of Formula (I) or pharmaceutically acceptable salt thereof every other day. In some embodiments, the method comprises administering to the patient about 80 mg to about 150 mg of the compound of Formula (I) or pharmaceutically acceptable salt thereof every other day. In some embodiments, the method comprises administering to the patient about 80 mg to about 100 mg of the compound of Formula (I) or pharmaceutically acceptable salt thereof every other day. In some embodiments, the method comprises administering to the patient about 90 mg to about 200 mg of the compound of Formula (I) or pharmaceutically acceptable salt thereof every other day. In some embodiments, the method comprises administering to the patient about 90 mg to about 150 mg of the compound of Formula (I) or pharmaceutically acceptable salt thereof every other day. In some embodiments, the method comprises administering to the patient about 90 mg to about 100 mg of the compound of Formula (I) or pharmaceutically acceptable salt thereof every other day. In some embodiments, the method comprises administering to the patient about 100 mg to about 200 mg of the compound of Formula (I) or pharmaceutically acceptable salt thereof every other day. In some embodiments, the method comprises administering to the patient about 100 mg to about 150 mg of the compound of Formula (I) or pharmaceutically acceptable salt thereof every other day.

In some embodiments, the method comprises administering to the patient about 50 mg of the compound of Formula (I) or pharmaceutically acceptable salt thereof every other day. In some embodiments, the method comprises administering to the patient about 55 mg of the compound of Formula (I) or pharmaceutically acceptable salt thereof every other day. In some embodiments, the method comprises administering to the patient about 60 mg of the compound of Formula (I) or pharmaceutically acceptable salt thereof every other day. In some embodiments, the method comprises administering to the patient about 65 mg of the compound of Formula (I) or pharmaceutically acceptable salt thereof every other day. In some embodiments, the method comprises administering to the patient about 70 mg of the compound of Formula (I) or pharmaceutically acceptable salt thereof every other day. In some embodiments, the method comprises administering to the patient about 75 mg of the compound embodiments, the method comprises administering to the patient about 80 mg of the compound of Formula (I) or pharmaceutically acceptable salt thereof every other day. In some embodiments, the method comprises administering to the patient about 85 mg of the compound of Formula (I) or pharmaceutically acceptable salt thereof every other day. In some embodiments, the method comprises administering to the patient about 90 mg of the compound of Formula (I) or pharmaceutically acceptable salt thereof every other day. In some embodiments, the method comprises administering to the patient about 95 mg of the compound of Formula (I) or pharmaceutically acceptable salt thereof every other day. In some embodiments, the method comprises administering to the patient about 100 mg of the compound of Formula (I) or pharmaceutically acceptable salt thereof every other day. In some embodiments, the method comprises administering to the patient about 105 mg of the compound of Formula (I) or pharmaceutically acceptable salt thereof every other day. In some embodiments, the method comprises administering to the patient about 110 mg of the compound of Formula (I) or pharmaceutically acceptable salt thereof every other day. In some embodiments, the method comprises administering to the patient about 115 mg of the compound of Formula (I) or pharmaceutically acceptable salt thereof every other day. In some embodiments, the method comprises administering to the patient about 120 mg of the compound of Formula (I) or pharmaceutically acceptable salt thereof every other day. In some embodiments, the method comprises administering to the patient about 125 mg of the compound of Formula (I) or pharmaceutically acceptable salt thereof every other day. In some embodiments, the method comprises administering to the patient about 130 mg of the compound of Formula (I) or pharmaceutically acceptable salt thereof every other day. In some embodiments, the method comprises administering to the patient about 135 mg of the compound of Formula (I) or pharmaceutically acceptable salt thereof every other day. In some embodiments, the method comprises administering to the patient about 140 mg of the compound of Formula (I) or pharmaceutically acceptable salt thereof every other day. In some embodiments, the method comprises administering to the patient about 145 mg of the compound of Formula (I) or pharmaceutically acceptable salt thereof every other day. In some embodiments, the method comprises administering to the patient about 150 mg of the compound of Formula (I) or pharmaceutically acceptable salt thereof every other day. In some embodiments, the method comprises administering to the patient about 155 mg of the compound of Formula (I) or pharmaceutically acceptable salt thereof every other day. In some embodiments, the method comprises administering to the patient about 160 mg of the compound of Formula (I) or pharmaceutically acceptable salt thereof every other day. In some embodiments, the method comprises administering to the patient about 165 mg of the compound of Formula (I) or pharmaceutically acceptable salt thereof every other day. In some embodiments, the method comprises administering to the patient about 170 mg of the compound of Formula (I) or pharmaceutically acceptable salt thereof every other day. In some embodiments, the method comprises administering to the patient about 175 mg of the compound of Formula (I) or pharmaceutically acceptable salt thereof every other day. In some embodiments, the method comprises administering to the patient about 180 mg of the compound of Formula (I) or pharmaceutically acceptable salt thereof every other day. In some embodiments, the method comprises administering to the patient about 185 mg of the compound of Formula (I) or pharmaceutically acceptable salt thereof every other day. In some embodiments, the method comprises administering to the patient about 190 mg of the compound of Formula (I) or pharmaceutically acceptable salt thereof every other day. In some embodiments, the method comprises administering to the patient about 195 mg of the compound of Formula (I) or pharmaceutically acceptable salt thereof every other day. In some embodiments, the method comprises administering to the patient about 200 mg of the compound of Formula (I) or pharmaceutically acceptable salt thereof every other day.

In some embodiments, a method of treating an endometrial adenocarcinoma described herein further comprises administering paclitaxel to the patient in need thereof. In one embodiment, the paclitaxel is intravenously administered to the patient, e.g., by intravenous infusion. The intravenously administered paclitaxel can be administered to a patient at various dose levels. In some embodiments, the paclitaxel is administered to the patient at about 50 mg/m² to about 150 mg/m². In some embodiments, the paclitaxel is administered to the patient at about 50 mg/m² to about 100 mg/m². In some embodiments, the paclitaxel is administered to the patient at about 60 mg/m² to about 80 mg/m². In some embodiments, the paclitaxel is administered to the patient at about 60 mg/m². In some embodiments, the paclitaxel is administered to the patient at about 80 mg/m². In some embodiments, the paclitaxel is administered to the patient at about 135 mg/m². In some embodiments, the paclitaxel is administered to the patient at about 175 mg/m².

Furthermore, the paclitaxel can be administered to the patient at various frequencies. In some embodiments, the paclitaxel is administered to the patient once per week (e.g., at about 60 mg/m² once per week or about 80 mg/m² once per week). In some embodiments, the paclitaxel is administered to the patient once every three weeks (e.g., at about 135 mg/m² once every three weeks or about 175 mg/m² once every three weeks). In some embodiments, the paclitaxel is administered to the patient by intravenous infusion once per week. In some embodiments, the paclitaxel is administered to the patient by intravenous infusion once per week for three consecutive weeks. In some embodiments, the method comprises administering paclitaxel to the patient by intravenous infusion for more than one cycle, wherein each cycle comprises administering paclitaxel to the patient by intravenous infusion once per week for three consecutive weeks (e.g., once per week for three consecutive weeks at about 60 mg/m² or about 80 mg/m²). In some embodiments, the method comprises not administering paclitaxel to the patient for at least one week between each cycle.

In some embodiments, the paclitaxel is administered to the patient by infusion once every three weeks. In some embodiments, the paclitaxel is administered to the patient for about 60 minutes once per week. In some embodiments, the paclitaxel is administered to the patient for about 3 hours once per week. In some embodiments, the paclitaxel is administered to the patient for about 60 minutes once every three weeks. In some embodiments, the paclitaxel is administered to the patient for about 3 hours once every three weeks. In some embodiments, the paclitaxel is administered to the patient by intravenous infusion for about 60 minutes once per week. In some embodiments, the paclitaxel is administered to the patient by infusion for about 3 hours once per week. In some embodiments, the paclitaxel is administered to the patient by intravenous infusion for about 60 minutes once every three weeks. In some embodiments, the paclitaxel is administered to the patient by infusion for about 3 hours once every three weeks. In some embodiments, the paclitaxel is continuously administered to the patient for about 60 minutes once per week. In some embodiments, the paclitaxel is continuously administered to the patient for about 3 hours once per week. In some embodiments, the paclitaxel is continuously administered to the patient for about 60 minutes once every three weeks. In some embodiments, the paclitaxel is continuously administered to the patient for about 3 hours once every three weeks. In some embodiments, the paclitaxel is continuously administered to the patient by infusion for about 60 minutes once per week. In some embodiments, the paclitaxel is continuously administered to the patient by intravenous infusion for about 3 hours once per week. In some embodiments, the paclitaxel is continuously administered to the patient by infusion for about 60 minutes once every three weeks. In some embodiments, the paclitaxel is continuously administered to the patient by intravenous infusion for about 3 hours once every three weeks.

In embodiments of the methods of treating an endometrial adenocarcinoma described herein, the disclosure also contemplates administration of the compound of Formula (I) or a pharmaceutically acceptable salt to a patient in need thereof prior to (neo-adjuvant) or after (adjuvant) surgery (e.g., surgical treatment of a solid tumor). In some embodiments, the compound of Formula (I) or a pharmaceutically acceptable salt is administered as an adjuvant. In some embodiments, the compound of Formula (I) or a pharmaceutically acceptable salt is administered as a neo-adjuvant. In some embodiments, the compound of Formula (I) or a pharmaceutically acceptable salt is administered as a neo-adjuvant and an adjuvant. In some embodiments, the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 100 years. In some embodiments, administration of the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 5 years. In some embodiments, administration of the compound of Formula (I) or a pharmaceutically acceptable salt is administered to a patient in need thereof as a neo-adjuvant for a period of 3 months to 6 months, followed by administration of the compound of Formula (I) or a pharmaceutically acceptable salt as an adjuvant for a period from 1 day to 5 years. In some embodiments, no administration of the compound of Formula (I) or a pharmaceutically acceptable salt occurs prior to surgery. In some embodiments, the compound of Formula (I) or a pharmaceutically acceptable salt is administered as an adjuvant for a period from 1 day to 100 years. In some embodiments, the compound of Formula (I) or a pharmaceutically acceptable salt is administered as an adjuvant for a period from 1 day to 5 years.

In another embodiment, provided herein is a method for achieving at least a partial response in a patient having endometrial adenocarcinoma, comprising administering to the patient an effective amount of the compound of Formula (I)

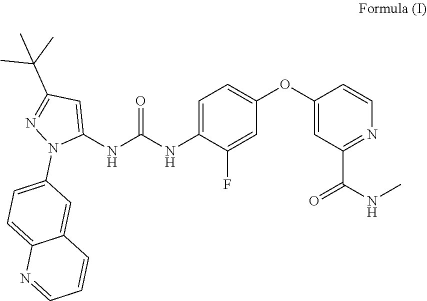 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In an embodiment, the patient has previously been administered at least one prior line of platinum-based therapy. In some embodiments, after 28 days or more of administering the compound of Formula (I) or pharmaceutically acceptable salt thereof to the patient, the patient has at least a partial response or a stable disease condition.

In some embodiments, the endometrial adenocarcinoma is characterized as having a serous histology. In some embodiments, the endometrial adenocarcinoma is characterized as having an endometroid histology. In some embodiments, the endometrial adenocarcinoma is a Grade 1 endometrioid adenocarcinoma. In some embodiments, the endometrial cancer is a Grade 2 endometrioid adenocarcinoma. In some embodiments, the endometrial cancer is a Grade 3 endometrioid adenocarcinoma.

In embodiments of the methods for achieving at least a partial response in a patient having endometrial adenocarcinoma described herein, the compound of Formula (I) or pharmaceutically acceptable salt thereof can be orally administered to the patient in need thereof. In certain embodiments, the compound of Formula (I) or pharmaceutically acceptable salt thereof is administered to the patient daily. In some embodiments, the method comprises administering to the patient about 50 mg to about 200 mg of the compound of Formula (I) or pharmaceutically acceptable salt thereof per day. In some embodiments, the method comprises administering to the patient about 50 mg to about 150 mg of the compound of Formula (I) or pharmaceutically acceptable salt thereof per day. In some embodiments, the method comprises administering to the patient about 50 mg to about 100 mg of the compound of Formula (I) or pharmaceutically acceptable salt thereof per day. In some embodiments, the method comprises administering to the patient about 60 mg to about 200 mg of the compound of Formula (I) or pharmaceutically acceptable salt thereof per day. In some embodiments, the method comprises administering to the patient about 60 mg to about 150 mg of the compound of Formula (I) or pharmaceutically acceptable salt thereof per day. In some embodiments, the method comprises administering to the patient about 60 mg to about 100 mg of the compound of Formula (I) or pharmaceutically acceptable salt thereof per day. In some embodiments, the method comprises administering to the patient about 70 mg to about 200 mg of the compound of Formula (I) or pharmaceutically acceptable salt thereof per day. In some embodiments, the method comprises administering to the patient about 70 mg to about 150 mg of the compound of Formula (I) or pharmaceutically acceptable salt thereof per day. In some embodiments, the method comprises administering to the patient about 70 mg to about 100 mg of the compound of Formula (I) or pharmaceutically acceptable salt thereof per day. In some embodiments, the method comprises administering to the patient about 80 mg to about 200 mg of the compound of Formula (I) or pharmaceutically acceptable salt thereof per day. In some embodiments, the method comprises administering to the patient about 80 mg to about 150 mg of the compound of Formula (I) or pharmaceutically acceptable salt thereof per day. In some embodiments, the method comprises administering to the patient about 80 mg to about 100 mg of the compound of Formula (I) or pharmaceutically acceptable salt thereof per day. In some embodiments, the method comprises administering to the patient about 90 mg to about 200 mg of the compound of Formula (I) or pharmaceutically acceptable salt thereof per day. In some embodiments, the method comprises administering to the patient about 90 mg to about 150 mg of the compound of Formula (I) or pharmaceutically acceptable salt thereof per day. In some embodiments, the method comprises administering to the patient about 90 mg to about 100 mg of the compound of Formula (I) or pharmaceutically acceptable salt thereof per day. In some embodiments, the method comprises administering to the patient about 100 mg to about 200 mg of the compound of Formula (I) or pharmaceutically acceptable salt thereof per day. In some embodiments, the method comprises administering to the patient about 100 mg to about 150 mg of the compound of Formula (I) or pharmaceutically acceptable salt thereof per day.

In some embodiments, the method comprises administering to the patient about 50 mg of the compound of Formula (I) or pharmaceutically acceptable salt thereof per day. In some embodiments, the method comprises administering to the patient about 55 mg of the compound of Formula (I) or pharmaceutically acceptable salt thereof per day. In some embodiments, the method comprises administering to the patient about 60 mg of the compound of Formula (I) or pharmaceutically acceptable salt thereof per day. In some embodiments, the method comprises administering to the patient about 65 mg of the compound of Formula (I) or pharmaceutically acceptable salt thereof per day. In some embodiments, the method comprises administering to the patient about 70 mg of the compound of Formula (I) or pharmaceutically acceptable salt thereof per day. In some embodiments, the method comprises administering to the patient about 75 mg of the compound of Formula (I) or pharmaceutically acceptable salt thereof per day. In some embodiments, the method comprises administering to the patient about 80 mg of the compound of Formula (I) or pharmaceutically acceptable salt thereof per day. In some embodiments, the method comprises administering to the patient about 85 mg of the compound of Formula (I) or pharmaceutically acceptable salt thereof per day. In some embodiments, the method comprises administering to the patient about 90 mg of the compound of Formula (I) or pharmaceutically acceptable salt thereof per day. In some embodiments, the method comprises administering to the patient about 95 mg of the compound of Formula (I) or pharmaceutically acceptable salt thereof per day. In some embodiments, the method comprises administering to the patient about 100 mg of the compound of Formula (I) or pharmaceutically acceptable salt thereof per day. In some embodiments, the method comprises administering to the patient about 105 mg of the compound of Formula (I) or pharmaceutically acceptable salt thereof per day. In some embodiments, the method comprises administering to the patient about 110 mg of the compound of Formula (I) or pharmaceutically acceptable salt thereof per day. In some embodiments, the method comprises administering to the patient about 115 mg of the compound of Formula (I) or pharmaceutically acceptable salt thereof per day. In some embodiments, the method comprises administering to the patient about 120 mg of the compound of Formula (I) or pharmaceutically acceptable salt thereof per day. In some embodiments, the method comprises administering to the patient about 125 mg of the compound of Formula (I) or pharmaceutically acceptable salt thereof per day. In some embodiments, the method comprises administering to the patient about 130 mg of the compound of Formula (I) or pharmaceutically acceptable salt thereof per day. In some embodiments, the method comprises administering to the patient about 135 mg of the compound of Formula (I) or pharmaceutically acceptable salt thereof per day. In some embodiments, the method comprises administering to the patient about 140 mg of the compound of Formula (I) or pharmaceutically acceptable salt thereof per day. In some embodiments, the method comprises administering to the patient about 145 mg of the compound of Formula (I) or pharmaceutically acceptable salt thereof per day. In some embodiments, the method comprises administering to the patient about 150 mg of the compound of Formula (I) or pharmaceutically acceptable salt thereof per day. In some embodiments, the method comprises administering to the patient about 155 mg of the compound of Formula (I) or pharmaceutically acceptable salt thereof per day. In some embodiments, the method comprises administering to the patient about 160 mg of the compound of Formula (I) or pharmaceutically acceptable salt thereof per day. In some embodiments, the method comprises administering to the patient about 165 mg of the compound of Formula (I) or pharmaceutically acceptable salt thereof per day. In some embodiments, the method comprises administering to the patient about 170 mg of the compound of Formula (I) or pharmaceutically acceptable salt thereof per day. In some embodiments, the method comprises administering to the patient about 175 mg of the compound of Formula (I) or pharmaceutically acceptable salt thereof per day. In some embodiments, the method comprises administering to the patient about 180 mg of the compound of Formula (I) or pharmaceutically acceptable salt thereof per day. In some embodiments, the method comprises administering to the patient about 185 mg of the compound of Formula (I) or pharmaceutically acceptable salt thereof per day. In some embodiments, the method comprises administering to the patient about 190 mg of the compound of Formula (I) or pharmaceutically acceptable salt thereof per day. In some embodiments, the method comprises administering to the patient about 195 mg of the compound of Formula (I) or pharmaceutically acceptable salt thereof per day. In some embodiments, the method comprises administering to the patient about 200 mg of the compound of Formula (I) or pharmaceutically acceptable salt thereof per day.

In some embodiments, the method comprises administering to the patient about 50 mg to about 200 mg of the compound of Formula (I) or pharmaceutically acceptable salt thereof once per day. In some embodiments, the method comprises administering to the patient about 50 mg to about 150 mg of the compound of Formula (I) or pharmaceutically acceptable salt thereof once per day. In some embodiments, the method comprises administering to the patient about 50 mg to about 100 mg of the compound of Formula (I) or pharmaceutically acceptable salt thereof once per day. In some embodiments, the method comprises administering to the patient about 60 mg to about 200 mg of the compound of Formula (I) or pharmaceutically acceptable salt thereof once per day. In some embodiments, the method comprises administering to the patient about 60 mg to about 150 mg of the compound of Formula (I) or pharmaceutically acceptable salt thereof once per day. In some embodiments, the method comprises administering to the patient about 60 mg to about 100 mg of the compound of Formula (I) or pharmaceutically acceptable salt thereof once per day. In some embodiments, the method comprises administering to the patient about 70 mg to about 200 mg of the compound of Formula (I) or pharmaceutically acceptable salt thereof once per day. In some embodiments, the method comprises administering to the patient about 70 mg to about 150 mg of the compound of Formula (I) or pharmaceutically acceptable salt thereof once per day. In some embodiments, the method comprises administering to the patient about 70 mg to about 100 mg of the compound of Formula (I) or pharmaceutically acceptable salt thereof once per day. In some embodiments, the method comprises administering to the patient about 80 mg to about 200 mg of the compound of Formula (I) or pharmaceutically acceptable salt thereof once per day. In some embodiments, the method comprises administering to the patient about 80 mg to about 150 mg of the compound of Formula (I) or pharmaceutically acceptable salt thereof once per day. In some embodiments, the method comprises administering to the patient about 80 mg to about 100 mg of the compound of Formula (I) or pharmaceutically acceptable salt thereof once per day. In some embodiments, the method comprises administering to the patient about 90 mg to about 200 mg of the compound of Formula (I) or pharmaceutically acceptable salt thereof once per day. In some embodiments, the method comprises administering to the patient about 90 mg to about 150 mg of the compound of Formula (I) or pharmaceutically acceptable salt thereof once per day. In some embodiments, the method comprises administering to the patient about 90 mg to about 100 mg of the compound of Formula (I) or pharmaceutically acceptable salt thereof once per day. In some embodiments, the method comprises administering to the patient about 100 mg to about 200 mg of the compound of Formula (I) or pharmaceutically acceptable salt thereof once per day. In some embodiments, the method comprises administering to the patient about 100 mg to about 150 mg of the compound of Formula (I) or pharmaceutically acceptable salt thereof once per day.

In some embodiments, the method comprises administering to the patient about 50 mg of the compound of Formula (I) or pharmaceutically acceptable salt thereof once per day. In some embodiments, the method comprises administering to the patient about 55 mg of the compound of Formula (I) or pharmaceutically acceptable salt thereof once per day. In some embodiments, the method comprises administering to the patient about 60 mg of the compound of Formula (I) or pharmaceutically acceptable salt thereof once per day. In some embodiments, the method comprises administering to the patient about 65 mg of the compound of Formula (I) or pharmaceutically acceptable salt thereof once per day. In some embodiments, the method comprises administering to the patient about 70 mg of the compound of Formula (I) or pharmaceutically acceptable salt thereof once per day. In some embodiments, the method comprises administering to the patient about 75 mg of the compound of Formula (I) or pharmaceutically acceptable salt thereof once per day. In some embodiments, the method comprises administering to the patient about 80 mg of the compound of Formula (I) or pharmaceutically acceptable salt thereof once per day. In some embodiments, the method comprises administering to the patient about 85 mg of the compound of Formula (I) or pharmaceutically acceptable salt thereof once per day. In some embodiments, the method comprises administering to the patient about 90 mg of the compound of Formula (I) or pharmaceutically acceptable salt thereof once per day. In some embodiments, the method comprises administering to the patient about 95 mg of the compound of Formula (I) or pharmaceutically acceptable salt thereof once per day. In some embodiments, the method comprises administering to the patient about 100 mg of the compound of Formula (I) or pharmaceutically acceptable salt thereof once per day. In some embodiments, the method comprises administering to the patient about 105 mg of the compound of Formula (I) or pharmaceutically acceptable salt thereof once per day. In some embodiments, the method comprises administering to the patient about 110 mg of the compound of Formula (I) or pharmaceutically acceptable salt thereof once per day. In some embodiments, the method comprises administering to the patient about 115 mg of the compound of Formula (I) or pharmaceutically acceptable salt thereof once per day. In some embodiments, the method comprises administering to the patient about 120 mg of the compound of Formula (I) or pharmaceutically acceptable salt thereof once per day. In some embodiments, the method comprises administering to the patient about 125 mg of the compound of Formula (I) or pharmaceutically acceptable salt thereof once per day. In some embodiments, the method comprises administering to the patient about 130 mg of the compound of Formula (I) or pharmaceutically acceptable salt thereof once per day. In some embodiments, the method comprises administering to the patient about 135 mg of the compound of Formula (I) or pharmaceutically acceptable salt thereof once per day. In some embodiments, the method comprises administering to the patient about 140 mg of the compound of Formula (I) or pharmaceutically acceptable salt thereof once per day. In some embodiments, the method comprises administering to the patient about 145 mg of the compound of Formula (I) or pharmaceutically acceptable salt thereof once per day. In some embodiments, the method comprises administering to the patient about 150 mg of the compound of Formula (I) or pharmaceutically acceptable salt thereof once per day. In some embodiments, the method comprises administering to the patient about 155 mg of the compound of Formula (I) or pharmaceutically acceptable salt thereof once per day. In some embodiments, the method comprises administering to the patient about 160 mg of the compound of Formula (I) or pharmaceutically acceptable salt thereof once per day. In some embodiments, the method comprises administering to the patient about 165 mg of the compound of Formula (I) or pharmaceutically acceptable salt thereof once per day. In some embodiments, the method comprises administering to the patient about 170 mg of the compound of Formula (I) or pharmaceutically acceptable salt thereof once per day. In some embodiments, the method comprises administering to the patient about 175 mg of the compound of Formula (I) or pharmaceutically acceptable salt thereof once per day. In some embodiments, the method comprises administering to the patient about 180 mg of the compound of Formula (I) or pharmaceutically acceptable salt thereof once per day. In some embodiments, the method comprises administering to the patient about 185 mg of the compound of Formula (I) or pharmaceutically acceptable salt thereof once per day. In some embodiments, the method comprises administering to the patient about 190 mg of the compound of Formula (I) or pharmaceutically acceptable salt thereof once per day. In some embodiments, the method comprises administering to the patient about 195 mg of the compound of Formula (I) or pharmaceutically acceptable salt thereof once per day. In some embodiments, the method comprises administering to the patient about 200 mg of the compound of Formula (I) or pharmaceutically acceptable salt thereof once per day.

In some embodiments, the method comprises administering to the patient about 50 mg to about 100 mg of the compound of Formula (I) or pharmaceutically acceptable salt thereof twice per day. In some embodiments, the method comprises administering to the patient about 60 mg to about 100 mg of the compound of Formula (I) or pharmaceutically acceptable salt thereof twice per day. In some embodiments, the method comprises administering to the patient about 70 mg to about 100 mg of the compound of Formula (I) or pharmaceutically acceptable salt thereof twice per day. In some embodiments, the method comprises administering to the patient about 80 mg to about 100 mg of the compound of Formula (I) or pharmaceutically acceptable salt thereof twice per day. In some embodiments, the method comprises administering to the patient about 90 mg to about 100 mg of the compound of Formula (I) or pharmaceutically acceptable salt thereof twice per day.

In some embodiments, the method comprises administering to the patient about 50 mg of the compound of Formula (I) or pharmaceutically acceptable salt thereof twice per day. In some embodiments, the method comprises administering to the patient about 55 mg of the compound of Formula (I) or pharmaceutically acceptable salt thereof twice per day. In some embodiments, the method comprises administering to the patient about 60 mg of the compound of Formula (I) or pharmaceutically acceptable salt thereof twice per day. In some embodiments, the method comprises administering to the patient about 65 mg of the compound of Formula (I) or pharmaceutically acceptable salt thereof twice per day. In some embodiments, the method comprises administering to the patient about 70 mg of the compound of Formula (I) or pharmaceutically acceptable salt thereof twice per day. In some embodiments, the method comprises administering to the patient about 75 mg of the compound of Formula (I) or pharmaceutically acceptable salt thereof twice per day. In some embodiments, the method comprises administering to the patient about 80 mg of the compound of Formula (I) or pharmaceutically acceptable salt thereof twice per day. In some embodiments, the method comprises administering to the patient about 85 mg of the compound of Formula (I) or pharmaceutically acceptable salt thereof twice per day. In some embodiments, the method comprises administering to the patient about 90 mg of the compound of Formula (I) or pharmaceutically acceptable salt thereof twice per day. In some embodiments, the method comprises administering to the patient about 95 mg of the compound of Formula (I) or pharmaceutically acceptable salt thereof twice per day. In some embodiments, the method comprises administering to the patient about 100 mg of the compound of Formula (I) or pharmaceutically acceptable salt thereof twice per day.

The compound of Formula (I) or pharmaceutically acceptable salt thereof may, in another embodiment, be administered to the patient on an intermittent basis in the methods for achieving at least a partial response in a patient having endometrial adenocarcinoma. In some embodiments, the compound of Formula (I) or pharmaceutically acceptable salt thereof is administered the patient every other day. In some embodiments, the compound of Formula (I) or pharmaceutically acceptable salt thereof is administered the patient every other day. In some embodiments, the compound of Formula (I) or pharmaceutically acceptable salt thereof is administered the patient once per week. In some embodiments, the compound of Formula (I) or pharmaceutically acceptable salt thereof is administered the patient twice per week. In some embodiments, the compound of Formula (I) or pharmaceutically acceptable salt thereof is administered the patient three times per week.

In some embodiments, the method comprises administering to the patient about 50 mg to about 200 mg of the compound of Formula (I) or pharmaceutically acceptable salt thereof once a week. In some embodiments, the method comprises administering to the patient about 50 mg to about 150 mg of the compound of Formula (I) or pharmaceutically acceptable salt thereof once a week. In some embodiments, the method comprises administering to the patient about 50 mg to about 100 mg of the compound of Formula (I) or pharmaceutically acceptable salt thereof once a week. In some embodiments, the method comprises administering to the patient about 60 mg to about 200 mg of the compound of Formula (I) or pharmaceutically acceptable salt thereof once a week. In some embodiments, the method comprises administering to the patient about 60 mg to about 150 mg of the compound of Formula (I) or pharmaceutically acceptable salt thereof once a week. In some embodiments, the method comprises administering to the patient about 60 mg to about 100 mg of the compound of Formula (I) or pharmaceutically acceptable salt thereof once a week. In some embodiments, the method comprises administering to the patient about 70 mg to about 200 mg of the compound of Formula (I) or pharmaceutically acceptable salt thereof once a week. In some embodiments, the method comprises administering to the patient about 70 mg to about 150 mg of the compound of Formula (I) or pharmaceutically acceptable salt thereof once a week. In some embodiments, the method comprises administering to the patient about 70 mg to about 100 mg of the compound of Formula (I) or pharmaceutically acceptable salt thereof once a week. In some embodiments, the method comprises administering to the patient about 80 mg to about 200 mg of the compound of Formula (I) or pharmaceutically acceptable salt thereof once a week. In some embodiments, the method comprises administering to the patient about 80 mg to about 150 mg of the compound of Formula (I) or pharmaceutically acceptable salt thereof once a week. In some embodiments, the method comprises administering to the patient about 80 mg to about 100 mg of the compound of Formula (I) or pharmaceutically acceptable salt thereof once a week. In some embodiments, the method comprises administering to the patient about 90 mg to about 200 mg of the compound of Formula (I) or pharmaceutically acceptable salt thereof once a week. In some embodiments, the method comprises administering to the patient about 90 mg to about 150 mg of the compound of Formula (I) or pharmaceutically acceptable salt thereof once a week. In some embodiments, the method comprises administering to the patient about 90 mg to about 100 mg of the compound of Formula (I) or pharmaceutically acceptable salt thereof once a week. In some embodiments, the method comprises administering to the patient about 100 mg to about 200 mg of the compound of Formula (I) or pharmaceutically acceptable salt thereof once a week. In some embodiments, the method comprises administering to the patient about 100 mg to about 150 mg of the compound of Formula (I) or pharmaceutically acceptable salt thereof once a week.

In some embodiments, the method comprises administering to the patient about 50 mg of the compound of Formula (I) or pharmaceutically acceptable salt thereof once a week. In some embodiments, the method comprises administering to the patient about 55 mg of the compound of Formula (I) or pharmaceutically acceptable salt thereof once a week. In some embodiments, the method comprises administering to the patient about 60 mg of the compound of Formula (I) or pharmaceutically acceptable salt thereof once a week. In some embodiments, the method comprises administering to the patient about 65 mg of the compound of Formula (I) or pharmaceutically acceptable salt thereof once a week. In some embodiments, the method comprises administering to the patient about 70 mg of the compound of Formula (I) or pharmaceutically acceptable salt thereof once a week. In some embodiments, the method comprises administering to the patient about 75 mg of the compound of Formula (I) or pharmaceutically acceptable salt thereof once a week. In some embodiments, the method comprises administering to the patient about 80 mg of the compound of Formula (I) or pharmaceutically acceptable salt thereof once a week. In some embodiments, the method comprises administering to the patient about 85 mg of the compound of Formula (I) or pharmaceutically acceptable salt thereof once a week. In some embodiments, the method comprises administering to the patient about 90 mg of the compound of Formula (I) or pharmaceutically acceptable salt thereof once a week. In some embodiments, the method comprises administering to the patient about 95 mg of the compound of Formula (I) or pharmaceutically acceptable salt thereof once a week. In some embodiments, the method comprises administering to the patient about 100 mg of the compound of Formula (I) or pharmaceutically acceptable salt thereof once a week. In some embodiments, the method comprises administering to the patient about 105 mg of the compound of Formula (I) or pharmaceutically acceptable salt thereof once a week. In some embodiments, the method comprises administering to the patient about 110 mg of the compound of Formula (I) or pharmaceutically acceptable salt thereof once a week. In some embodiments, the method comprises administering to the patient about 115 mg of the compound of Formula (I) or pharmaceutically acceptable salt thereof once a week. In some embodiments, the method comprises administering to the patient about 120 mg of the compound of Formula (I) or pharmaceutically acceptable salt thereof once a week. In some embodiments, the method comprises administering to the patient about 125 mg of the compound of Formula (I) or pharmaceutically acceptable salt thereof once a week. In some embodiments, the method comprises administering to the patient about 130 mg of the compound of Formula (I) or pharmaceutically acceptable salt thereof once a week. In some embodiments, the method comprises administering to the patient about 135 mg of the compound of Formula (I) or pharmaceutically acceptable salt thereof once a week. In some embodiments, the method comprises administering to the patient about 140 mg of the compound of Formula (I) or pharmaceutically acceptable salt thereof once a week. In some embodiments, the method comprises administering to the patient about 145 mg of the compound of Formula (I) or pharmaceutically acceptable salt thereof once a week. In some embodiments, the method comprises administering to the patient about 150 mg of the compound of Formula (I) or pharmaceutically acceptable salt thereof once a week. In some embodiments, the method comprises administering to the patient about 155 mg of the compound of Formula (I) or pharmaceutically acceptable salt thereof once a week. In some embodiments, the method comprises administering to the patient about 160 mg of the compound of Formula (I) or pharmaceutically acceptable salt thereof once a week. In some embodiments, the method comprises administering to the patient about 165 mg of the compound of Formula (I) or pharmaceutically acceptable salt thereof once a week. In some embodiments, the method comprises administering to the patient about 170 mg of the compound of Formula (I) or pharmaceutically acceptable salt thereof once a week. In some embodiments, the method comprises administering to the patient about 175 mg of the compound of Formula (I) or pharmaceutically acceptable salt thereof once a week. In some embodiments, the method comprises administering to the patient about 180 mg of the compound of Formula (I) or pharmaceutically acceptable salt thereof once a week. In some embodiments, the method comprises administering to the patient about 185 mg of the compound of Formula (I) or pharmaceutically acceptable salt thereof once a week. In some embodiments, the method comprises administering to the patient about 190 mg of the compound of Formula (I) or pharmaceutically acceptable salt thereof once a week. In some embodiments, the method comprises administering to the patient about 195 mg of the compound of Formula (I) or pharmaceutically acceptable salt thereof once a week. In some embodiments, the method comprises administering to the patient about 200 mg of the compound of Formula (I) or pharmaceutically acceptable salt thereof once a week.

In some embodiments, the method comprises administering to the patient about 50 mg to about 200 mg of the compound of Formula (I) or pharmaceutically acceptable salt thereof twice a week. In some embodiments, the method comprises administering to the patient about 50 mg to about 150 mg of the compound of Formula (I) or pharmaceutically acceptable salt thereof twice a week. In some embodiments, the method comprises administering to the patient about 50 mg to about 100 mg of the compound of Formula (I) or pharmaceutically acceptable salt thereof twice a week. In some embodiments, the method comprises administering to the patient about 60 mg to about 200 mg of the compound of Formula (I) or pharmaceutically acceptable salt thereof twice a week. In some embodiments, the method comprises administering to the patient about 60 mg to about 150 mg of the compound of Formula (I) or pharmaceutically acceptable salt thereof twice a week. In some embodiments, the method comprises administering to the patient about 60 mg to about 100 mg of the compound of Formula (I) or pharmaceutically acceptable salt thereof twice a week. In some embodiments, the method comprises administering to the patient about 70 mg to about 200 mg of the compound of Formula (I) or pharmaceutically acceptable salt thereof twice a week. In some embodiments, the method comprises administering to the patient about 70 mg to about 150 mg of the compound of Formula (I) or pharmaceutically acceptable salt thereof twice a week. In some embodiments, the method comprises administering to the patient about 70 mg to about 100 mg of the compound of Formula (I) or pharmaceutically acceptable salt thereof twice a week. In some embodiments, the method comprises administering to the patient about 80 mg to about 200 mg of the compound of Formula (I) or pharmaceutically acceptable salt thereof twice a week. In some embodiments, the method comprises administering to the patient about 80 mg to about 150 mg of the compound of Formula (I) or pharmaceutically acceptable salt thereof twice a week. In some embodiments, the method comprises administering to the patient about 80 mg to about 100 mg of the compound of Formula (I) or pharmaceutically acceptable salt thereof twice a week. In some embodiments, the method comprises administering to the patient about 90 mg to about 200 mg of the compound of Formula (I) or pharmaceutically acceptable salt thereof twice a week. In some embodiments, the method comprises administering to the patient about 90 mg to about 150 mg of the compound of Formula (I) or pharmaceutically acceptable salt thereof twice a week. In some embodiments, the method comprises administering to the patient about 90 mg to about 100 mg of the compound of Formula (I) or pharmaceutically acceptable salt thereof twice a week. In some embodiments, the method comprises administering to the patient about 100 mg to about 200 mg of the compound of Formula (I) or pharmaceutically acceptable salt thereof twice a week. In some embodiments, the method comprises administering to the patient about 100 mg to about 150 mg of the compound of Formula (I) or pharmaceutically acceptable salt thereof twice a week.

In some embodiments, the method comprises administering to the patient about 50 mg of the compound of Formula (I) or pharmaceutically acceptable salt thereof twice a week. In some embodiments, the method comprises administering to the patient about 55 mg of the compound of Formula (I) or pharmaceutically acceptable salt thereof twice a week. In some embodiments, the method comprises administering to the patient about 60 mg of the compound of Formula (I) or pharmaceutically acceptable salt thereof twice a week. In some embodiments, the method comprises administering to the patient about 65 mg of the compound of Formula (I) or pharmaceutically acceptable salt thereof twice a week. In some embodiments, the method comprises administering to the patient about 70 mg of the compound of Formula (I) or pharmaceutically acceptable salt thereof twice a week. In some embodiments, the method comprises administering to the patient about 75 mg of the compound of Formula (I) or pharmaceutically acceptable salt thereof twice a week. In some embodiments, the method comprises administering to the patient about 80 mg of the compound of Formula (I) or pharmaceutically acceptable salt thereof twice a week. In some embodiments, the method comprises administering to the patient about 85 mg of the compound of Formula (I) or pharmaceutically acceptable salt thereof twice a week. In some embodiments, the method comprises administering to the patient about 90 mg of the compound of Formula (I) or pharmaceutically acceptable salt thereof twice a week. In some embodiments, the method comprises administering to the patient about 95 mg of the compound of Formula (I) or pharmaceutically acceptable salt thereof twice a week. In some embodiments, the method comprises administering to the patient about 100 mg of the compound of Formula (I) or pharmaceutically acceptable salt thereof twice a week. In some embodiments, the method comprises administering to the patient about 105 mg of the compound of Formula (I) or pharmaceutically acceptable salt thereof twice a week. In some embodiments, the method comprises administering to the patient about 110 mg of the compound of Formula (I) or pharmaceutically acceptable salt thereof twice a week. In some embodiments, the method comprises administering to the patient about 115 mg of the compound of Formula (I) or pharmaceutically acceptable salt thereof twice a week. In some embodiments, the method comprises administering to the patient about 120 mg of the compound of Formula (I) or pharmaceutically acceptable salt thereof twice a week. In some embodiments, the method comprises administering to the patient about 125 mg of the compound of Formula (I) or pharmaceutically acceptable salt thereof twice a week. In some embodiments, the method comprises administering to the patient about 130 mg of the compound of Formula (I) or pharmaceutically acceptable salt thereof twice a week. In some embodiments, the method comprises administering to the patient about 135 mg of the compound of Formula (I) or pharmaceutically acceptable salt thereof twice a week. In some embodiments, the method comprises administering to the patient about 140 mg of the compound of Formula (I) or pharmaceutically acceptable salt thereof twice a week. In some embodiments, the method comprises administering to the patient about 145 mg of the compound of Formula (I) or pharmaceutically acceptable salt thereof twice a week. In some embodiments, the method comprises administering to the patient about 150 mg of the compound of Formula (I) or pharmaceutically acceptable salt thereof twice a week. In some embodiments, the method comprises administering to the patient about 155 mg of the compound of Formula (I) or pharmaceutically acceptable salt thereof twice a week. In some embodiments, the method comprises administering to the patient about 160 mg of the compound of Formula (I) or pharmaceutically acceptable salt thereof twice a week. In some embodiments, the method comprises administering to the patient about 165 mg of the compound of Formula (I) or pharmaceutically acceptable salt thereof twice a week. In some embodiments, the method comprises administering to the patient about 170 mg of the compound of Formula (I) or pharmaceutically acceptable salt thereof twice a week. In some embodiments, the method comprises administering to the patient about 175 mg of the compound of Formula (I) or pharmaceutically acceptable salt thereof twice a week. In some embodiments, the method comprises administering to the patient about 180 mg of the compound of Formula (I) or pharmaceutically acceptable salt thereof twice a week. In some embodiments, the method comprises administering to the patient about 185 mg of the compound of Formula (I) or pharmaceutically acceptable salt thereof twice a week. In some embodiments, the method comprises administering to the patient about 190 mg of the compound of Formula (I) or pharmaceutically acceptable salt thereof twice a week. In some embodiments, the method comprises administering to the patient about 195 mg of the compound of Formula (I) or pharmaceutically acceptable salt thereof twice a week. In some embodiments, the method comprises administering to the patient about 200 mg of the compound of Formula (I) or pharmaceutically acceptable salt thereof twice a week.

In some embodiments, the method comprises administering to the patient about 50 mg to about 200 mg of the compound of Formula (I) or pharmaceutically acceptable salt thereof three times a week. In some embodiments, the method comprises administering to the patient about 50 mg to about 150 mg of the compound of Formula (I) or pharmaceutically acceptable salt thereof three times a week. In some embodiments, the method comprises administering to the patient about 50 mg to about 100 mg of the compound of Formula (I) or pharmaceutically acceptable salt thereof three times a week. In some embodiments, the method comprises administering to the patient about 60 mg to about 200 mg of the compound of Formula (I) or pharmaceutically acceptable salt thereof three times a week. In some embodiments, the method comprises administering to the patient about 60 mg to about 150 mg of the compound of Formula (I) or pharmaceutically acceptable salt thereof three times a week. In some embodiments, the method comprises administering to the patient about 60 mg to about 100 mg of the compound of Formula (I) or pharmaceutically acceptable salt thereof three times a week. In some embodiments, the method comprises administering to the patient about 70 mg to about 200 mg of the compound of Formula (I) or pharmaceutically acceptable salt thereof three times a week. In some embodiments, the method comprises administering to the patient about 70 mg to about 150 mg of the compound of Formula (I) or pharmaceutically acceptable salt thereof three times a week. In some embodiments, the method comprises administering to the patient about 70 mg to about 100 mg of the compound of Formula (I) or pharmaceutically acceptable salt thereof three times a week. In some embodiments, the method comprises administering to the patient about 80 mg to about 200 mg of the compound of Formula (I) or pharmaceutically acceptable salt thereof three times a week. In some embodiments, the method comprises administering to the patient about 80 mg to about 150 mg of the compound of Formula (I) or pharmaceutically acceptable salt thereof three times a week. In some embodiments, the method comprises administering to the patient about 80 mg to about 100 mg of the compound of Formula (I) or pharmaceutically acceptable salt thereof three times a week. In some embodiments, the method comprises administering to the patient about 90 mg to about 200 mg of the compound of Formula (I) or pharmaceutically acceptable salt thereof three times a week. In some embodiments, the method comprises administering to the patient about 90 mg to about 150 mg of the compound of Formula (I) or pharmaceutically acceptable salt thereof three times a week. In some embodiments, the method comprises administering to the patient about 90 mg to about 100 mg of the compound of Formula (I) or pharmaceutically acceptable salt thereof three times a week. In some embodiments, the method comprises administering to the patient about 100 mg to about 200 mg of the compound of Formula (I) or pharmaceutically acceptable salt thereof three times a week. In some embodiments, the method comprises administering to the patient about 100 mg to about 150 mg of the compound of Formula (I) or pharmaceutically acceptable salt thereof three times a week.

In some embodiments, the method comprises administering to the patient about 50 mg of the compound of Formula (I) or pharmaceutically acceptable salt thereof three times a week. In some embodiments, the method comprises administering to the patient about 55 mg of the compound of Formula (I) or pharmaceutically acceptable salt thereof three times a week. In some embodiments, the method comprises administering to the patient about 60 mg of the compound of Formula (I) or pharmaceutically acceptable salt thereof three times a week. In some embodiments, the method comprises administering to the patient about 65 mg of the compound of Formula (I) or pharmaceutically acceptable salt thereof three times a week. In some embodiments, the method comprises administering to the patient about 70 mg of the compound of Formula (I) or pharmaceutically acceptable salt thereof three times a week. In some embodiments, the method comprises administering to the patient about 75 mg of the compound of Formula (I) or pharmaceutically acceptable salt thereof three times a week. In some embodiments, the method comprises administering to the patient about 80 mg of the compound of Formula (I) or pharmaceutically acceptable salt thereof three times a week. In some embodiments, the method comprises administering to the patient about 85 mg of the compound of Formula (I) or pharmaceutically acceptable salt thereof three times a week. In some embodiments, the method comprises administering to the patient about 90 mg of the compound of Formula (I) or pharmaceutically acceptable salt thereof three times a week. In some embodiments, the method comprises administering to the patient about 95 mg of the compound of Formula (I) or pharmaceutically acceptable salt thereof three times a week. In some embodiments, the method comprises administering to the patient about 100 mg of the compound of Formula (I) or pharmaceutically acceptable salt thereof three times a week. In some embodiments, the method comprises administering to the patient about 105 mg of the compound of Formula (I) or pharmaceutically acceptable salt thereof three times a week. In some embodiments, the method comprises administering to the patient about 110 mg of the compound of Formula (I) or pharmaceutically acceptable salt thereof three times a week. In some embodiments, the method comprises administering to the patient about 115 mg of the compound of Formula (I) or pharmaceutically acceptable salt thereof three times a week. In some embodiments, the method comprises administering to the patient about 120 mg of the compound of Formula (I) or pharmaceutically acceptable salt thereof three times a week. In some embodiments, the method comprises administering to the patient about 125 mg of the compound of Formula (I) or pharmaceutically acceptable salt thereof three times a week. In some embodiments, the method comprises administering to the patient about 130 mg of the compound of Formula (I) or pharmaceutically acceptable salt thereof three times a week. In some embodiments, the method comprises administering to the patient about 135 mg of the compound of Formula (I) or pharmaceutically acceptable salt thereof three times a week. In some embodiments, the method comprises administering to the patient about 140 mg of the compound of Formula (I) or pharmaceutically acceptable salt thereof three times a week. In some embodiments, the method comprises administering to the patient about 145 mg of the compound of Formula (I) or pharmaceutically acceptable salt thereof three times a week. In some embodiments, the method comprises administering to the patient about 150 mg of the compound of Formula (I) or pharmaceutically acceptable salt thereof three times a week. In some embodiments, the method comprises administering to the patient about 155 mg of the compound of Formula (I) or pharmaceutically acceptable salt thereof three times a week. In some embodiments, the method comprises administering to the patient about 160 mg of the compound of Formula (I) or pharmaceutically acceptable salt thereof three times a week. In some embodiments, the method comprises administering to the patient about 165 mg of the compound of Formula (I) or pharmaceutically acceptable salt thereof three times a week. In some embodiments, the method comprises administering to the patient about 170 mg of the compound of Formula (I) or pharmaceutically acceptable salt thereof three times a week. In some embodiments, the method comprises administering to the patient about 175 mg of the compound of Formula (I) or pharmaceutically acceptable salt thereof three times a week. In some embodiments, the method comprises administering to the patient about 180 mg of the compound of Formula (I) or pharmaceutically acceptable salt thereof three times a week. In some embodiments, the method comprises administering to the patient about 185 mg of the compound of Formula (I) or pharmaceutically acceptable salt thereof three times a week. In some embodiments, the method comprises administering to the patient about 190 mg of the compound of Formula (I) or pharmaceutically acceptable salt thereof three times a week. In some embodiments, the method comprises administering to the patient about 195 mg of the compound of Formula (I) or pharmaceutically acceptable salt thereof three times a week. In some embodiments, the method comprises administering to the patient about 200 mg of the compound of Formula (I) or pharmaceutically acceptable salt thereof three times a week.

In some embodiments, the method comprises administering to the patient about 50 mg to about 200 mg of the compound of Formula (I) or pharmaceutically acceptable salt thereof every other day. In some embodiments, the method comprises administering to the patient about 50 mg to about 150 mg of the compound of Formula (I) or pharmaceutically acceptable salt thereof every other day. In some embodiments, the method comprises administering to the patient about 50 mg to about 100 mg of the compound of Formula (I) or pharmaceutically acceptable salt thereof every other day. In some embodiments, the method comprises administering to the patient about 60 mg to about 200 mg of the compound of Formula (I) or pharmaceutically acceptable salt thereof every other day. In some embodiments, the method comprises administering to the patient about 60 mg to about 150 mg of the compound of Formula (I) or pharmaceutically acceptable salt thereof every other day. In some embodiments, the method comprises administering to the patient about 60 mg to about 100 mg of the compound of Formula (I) or pharmaceutically acceptable salt thereof every other day. In some embodiments, the method comprises administering to the patient about 70 mg to about 200 mg of the compound of Formula (I) or pharmaceutically acceptable salt thereof every other day. In some embodiments, the method comprises administering to the patient about 70 mg to about 150 mg of the compound of Formula (I) or pharmaceutically acceptable salt thereof every other day. In some embodiments, the method comprises administering to the patient about 70 mg to about 100 mg of the compound of Formula (I) or pharmaceutically acceptable salt thereof every other day. In some embodiments, the method comprises administering to the patient about 80 mg to about 200 mg of the compound of Formula (I) or pharmaceutically acceptable salt thereof every other day. In some embodiments, the method comprises administering to the patient about 80 mg to about 150 mg of the compound of Formula (I) or pharmaceutically acceptable salt thereof every other day. In some embodiments, the method comprises administering to the patient about 80 mg to about 100 mg of the compound of Formula (I) or pharmaceutically acceptable salt thereof every other day. In some embodiments, the method comprises administering to the patient about 90 mg to about 200 mg of the compound of Formula (I) or pharmaceutically acceptable salt thereof every other day. In some embodiments, the method comprises administering to the patient about 90 mg to about 150 mg of the compound of Formula (I) or pharmaceutically acceptable salt thereof every other day. In some embodiments, the method comprises administering to the patient about 90 mg to about 100 mg of the compound of Formula (I) or pharmaceutically acceptable salt thereof every other day. In some embodiments, the method comprises administering to the patient about 100 mg to about 200 mg of the compound of Formula (I) or pharmaceutically acceptable salt thereof every other day. In some embodiments, the method comprises administering to the patient about 100 mg to about 150 mg of the compound of Formula (I) or pharmaceutically acceptable salt thereof every other day.

In some embodiments, the method comprises administering to the patient about 50 mg of the compound of Formula (I) or pharmaceutically acceptable salt thereof every other day. In some embodiments, the method comprises administering to the patient about 55 mg of the compound of Formula (I) or pharmaceutically acceptable salt thereof every other day. In some embodiments, the method comprises administering to the patient about 60 mg of the compound of Formula (I) or pharmaceutically acceptable salt thereof every other day. In some embodiments, the method comprises administering to the patient about 65 mg of the compound of Formula (I) or pharmaceutically acceptable salt thereof every other day. In some embodiments, the method comprises administering to the patient about 70 mg of the compound of Formula (I) or pharmaceutically acceptable salt thereof every other day. In some embodiments, the method comprises administering to the patient about 75 mg of the compound of Formula (I) or pharmaceutically acceptable salt thereof every other day. In some embodiments, the method comprises administering to the patient about 80 mg of the compound of Formula (I) or pharmaceutically acceptable salt thereof every other day. In some embodiments, the method comprises administering to the patient about 85 mg of the compound of Formula (I) or pharmaceutically acceptable salt thereof every other day. In some embodiments, the method comprises administering to the patient about 90 mg of the compound of Formula (I) or pharmaceutically acceptable salt thereof every other day. In some embodiments, the method comprises administering to the patient about 95 mg of the compound of Formula (I) or pharmaceutically acceptable salt thereof every other day. In some embodiments, the method comprises administering to the patient about 100 mg of the compound of Formula (I) or pharmaceutically acceptable salt thereof every other day. In some embodiments, the method comprises administering to the patient about 105 mg of the compound of Formula (I) or pharmaceutically acceptable salt thereof every other day. In some embodiments, the method comprises administering to the patient about 110 mg of the compound of Formula (I) or pharmaceutically acceptable salt thereof every other day. In some embodiments, the method comprises administering to the patient about 115 mg of the compound of Formula (I) or pharmaceutically acceptable salt thereof every other day. In some embodiments, the method comprises administering to the patient about 120 mg of the compound embodiments, the method comprises administering to the patient about 125 mg of the compound of Formula (I) or pharmaceutically acceptable salt thereof every other day. In some embodiments, the method comprises administering to the patient about 130 mg of the compound of Formula (I) or pharmaceutically acceptable salt thereof every other day. In some embodiments, the method comprises administering to the patient about 135 mg of the compound of Formula (I) or pharmaceutically acceptable salt thereof every other day. In some embodiments, the method comprises administering to the patient about 140 mg of the compound of Formula (I) or pharmaceutically acceptable salt thereof every other day. In some embodiments, the method comprises administering to the patient about 145 mg of the compound of Formula (I) or pharmaceutically acceptable salt thereof every other day. In some embodiments, the method comprises administering to the patient about 150 mg of the compound of Formula (I) or pharmaceutically acceptable salt thereof every other day. In some embodiments, the method comprises administering to the patient about 155 mg of the compound of Formula (I) or pharmaceutically acceptable salt thereof every other day. In some embodiments, the method comprises administering to the patient about 160 mg of the compound of Formula (I) or pharmaceutically acceptable salt thereof every other day. In some embodiments, the method comprises administering to the patient about 165 mg of the compound of Formula (I) or pharmaceutically acceptable salt thereof every other day. In some embodiments, the method comprises administering to the patient about 170 mg of the compound of Formula (I) or pharmaceutically acceptable salt thereof every other day. In some embodiments, the method comprises administering to the patient about 175 mg of the compound of Formula (I) or pharmaceutically acceptable salt thereof every other day. In some embodiments, the method comprises administering to the patient about 180 mg of the compound of Formula (I) or pharmaceutically acceptable salt thereof every other day. In some embodiments, the method comprises administering to the patient about 185 mg of the compound of Formula (I) or pharmaceutically acceptable salt thereof every other day. In some embodiments, the method comprises administering to the patient about 190 mg of the compound of Formula (I) or pharmaceutically acceptable salt thereof every other day. In some embodiments, the method comprises administering to the patient about 195 mg of the compound of Formula (I) or pharmaceutically acceptable salt thereof every other day. In some embodiments, the method comprises administering to the patient about 200 mg of the compound of Formula (I) or pharmaceutically acceptable salt thereof every other day.

In some embodiments, a method of achieving at least a partial response in a patient having an endometrial adenocarcinoma described herein further comprises administering paclitaxel to the patient in need thereof. In some embodiments, the paclitaxel is intravenously administered to the patient, e.g., by intravenous infusion. The intravenously administered paclitaxel can be administered to a patient at various dose levels. In some embodiments, the paclitaxel is administered to the patient at about 50 mg/m² to about 150 mg/m². In some embodiments, the paclitaxel is administered to the patient at about 50 mg/m² to about 100 mg/m². In some embodiments, the paclitaxel is administered to the patient at about 60 mg/m² to about 80 mg/m². In some embodiments, the paclitaxel is administered to the patient at about 60 mg/m². In some embodiments, the paclitaxel is administered to the patient at about 80 mg/m². In some embodiments, the paclitaxel is administered to the patient at about 135 mg/m². In some embodiments, the paclitaxel is administered to the patient at about 175 mg/m².

Furthermore, the paclitaxel can be administered to the patient at various frequencies. In some embodiments, the paclitaxel is administered to the patient once per week (e.g., at about 60 mg/m² once per week or about 80 mg/m² once per week). In some embodiments, the paclitaxel is administered to the patient once every three weeks (e.g., at about 135 mg/m² once every three weeks or about 175 mg/m² once every three weeks). In some embodiments, the paclitaxel is administered to the patient by intravenous infusion once per week. In some embodiments, the paclitaxel is administered to the patient by intravenous infusion once per week for three consecutive weeks. In some embodiments, the method comprises administering paclitaxel to the patient by intravenous infusion for more than one cycle, wherein each cycle comprises administering paclitaxel to the patient by intravenous infusion once per week for three consecutive weeks (e.g., once per week for three consecutive weeks at about 60 mg/m² or about 80 mg/m²). In some embodiments, the method comprises not administering paclitaxel to the patient for at least one week between each cycle.

In some embodiments, the paclitaxel is administered to the patient by infusion once every three weeks. In some embodiments, the paclitaxel is administered to the patient for about 60 minutes once per week. In some embodiments, the paclitaxel is administered to the patient for about 3 hours once per week. In some embodiments, the paclitaxel is administered to the patient for about 60 minutes once every three weeks. In some embodiments, the paclitaxel is administered to the patient for about 3 hours once every three weeks. In some embodiments, the paclitaxel is administered to the patient by intravenous infusion for about 60 minutes once per week. In some embodiments, the paclitaxel is administered to the patient by infusion for about 3 hours once per week. In some embodiments, the paclitaxel is administered to the patient by intravenous infusion for about 60 minutes once every three weeks. In some embodiments, the paclitaxel is administered to the patient by infusion for about 3 hours once every three weeks. In some embodiments, the paclitaxel is continuously administered to the patient for about 60 minutes once per week. In some embodiments, the paclitaxel is continuously administered to the patient for about 3 hours once per week. In some embodiments, the paclitaxel is continuously administered to the patient for about 60 minutes once every three weeks. In some embodiments, the paclitaxel is continuously administered to the patient for about 3 hours once every three weeks. In some embodiments, the paclitaxel is continuously administered to the patient by infusion for about 60 minutes once per week. In some embodiments, the paclitaxel is continuously administered to the patient by intravenous infusion for about 3 hours once per week. In some embodiments, the paclitaxel is continuously administered to the patient by infusion for about 60 minutes once every three weeks. In some embodiments, the paclitaxel is continuously administered to the patient by intravenous infusion for about 3 hours once every three weeks.

In embodiments of the methods of achieving at least a partial response in a patient having an endometrial adenocarcinoma described herein, the disclosure also contemplates administration of the compound of Formula (I) or a pharmaceutically acceptable salt to a patient in need thereof prior to (neo-adjuvant) or after (adjuvant) surgery (e.g., surgical treatment of a solid tumor). In some embodiments, the compound of Formula (I) or a pharmaceutically acceptable salt is administered as an adjuvant. In some embodiments, the compound of Formula (I) or a pharmaceutically acceptable salt is administered as a neo-adjuvant. In some embodiments, the compound of Formula (I) or a pharmaceutically acceptable salt is administered as a neo-adjuvant and an adjuvant. In some embodiments, the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 100 years. In some embodiments, administration of the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 5 years. In some embodiments, administration of the compound of Formula (I) or a pharmaceutically acceptable salt is administered to a patient in need thereof as a neo-adjuvant for a period of 3 months to 6 months, followed by administration of the compound of Formula (I) or a pharmaceutically acceptable salt as an adjuvant for a period from 1 day to 5 years. In some embodiments, no administration of the compound of Formula (I) or a pharmaceutically acceptable salt occurs prior to surgery. In some embodiments, the compound of Formula (I) or a pharmaceutically acceptable salt is administered as an adjuvant for a period from 1 day to 100 years. In some embodiments, the compound of Formula (I) or a pharmaceutically acceptable salt is administered as an adjuvant for a period from 1 day to 5 years.

In another embodiment, provided herein is a method for achieving a stable disease condition in a patient having endometrial adenocarcinoma, comprising administering to the patient an effective amount of the compound of Formula (I)

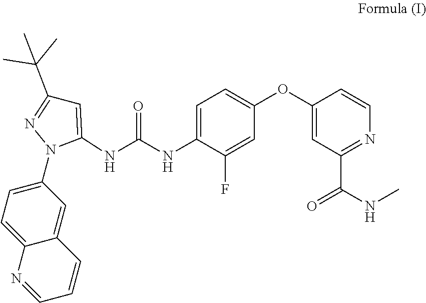 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In an embodiment, the patient has previously been administered at least one prior line of platinum-based therapy. In some embodiments, after 28 days or more of administering the compound of Formula (I) or pharmaceutically acceptable salt thereof to the patient, the patient has at least a partial response or a stable disease condition.

In some embodiments, the endometrial adenocarcinoma is characterized as having a serous histology. In some embodiments, the endometrial adenocarcinoma is characterized as having an endometroid histology. In some embodiments, the endometrial adenocarcinoma is a Grade 1 endometrioid adenocarcinoma. In some embodiments, the endometrial cancer is a Grade 2 endometrioid adenocarcinoma. In some embodiments, the endometrial cancer is a Grade 3 endometrioid adenocarcinoma.

In embodiments of the methods for achieving a stable disease condition in a patient having endometrial adenocarcinoma described herein, the compound of Formula (I) or pharmaceutically acceptable salt thereof can be orally administered to the patient in need thereof. In certain embodiments, the compound of Formula (I) or pharmaceutically acceptable salt thereof is administered to the patient daily. In some embodiments, the method comprises administering to the patient about 50 mg to about 200 mg of the compound of Formula (I) or pharmaceutically acceptable salt thereof per day. In some embodiments, the method comprises administering to the patient about 50 mg to about 150 mg of the compound of Formula (I) or pharmaceutically acceptable salt thereof per day. In some embodiments, the method comprises administering to the patient about 50 mg to about 100 mg of the compound of Formula (I) or pharmaceutically acceptable salt thereof per day. In some embodiments, the method comprises administering to the patient about 60 mg to about 200 mg of the compound of Formula (I) or pharmaceutically acceptable salt thereof per day. In some embodiments, the method comprises administering to the patient about 60 mg to about 150 mg of the compound of Formula (I) or pharmaceutically acceptable salt thereof per day. In some embodiments, the method comprises administering to the patient about 60 mg to about 100 mg of the compound of Formula (I) or pharmaceutically acceptable salt thereof per day. In some embodiments, the method comprises administering to the patient about 70 mg to about 200 mg of the compound of Formula (I) or pharmaceutically acceptable salt thereof per day. In some embodiments, the method comprises administering to the patient about 70 mg to about 150 mg of the compound of Formula (I) or pharmaceutically acceptable salt thereof per day. In some embodiments, the method comprises administering to the patient about 70 mg to about 100 mg of the compound of Formula (I) or pharmaceutically acceptable salt thereof per day. In some embodiments, the method comprises administering to the patient about 80 mg to about 200 mg of the compound of Formula (I) or pharmaceutically acceptable salt thereof per day. In some embodiments, the method comprises administering to the patient about 80 mg to about 150 mg of the compound of Formula (I) or pharmaceutically acceptable salt thereof per day. In some embodiments, the method comprises administering to the patient about 80 mg to about 100 mg of the compound of Formula (I) or pharmaceutically acceptable salt thereof per day. In some embodiments, the method comprises administering to the patient about 90 mg to about 200 mg of the compound of Formula (I) or pharmaceutically acceptable salt thereof per day. In some embodiments, the method comprises administering to the patient about 90 mg to about 150 mg of the compound of Formula (I) or pharmaceutically acceptable salt thereof per day. In some embodiments, the method comprises administering to the patient about 90 mg to about 100 mg of the compound of Formula (I) or pharmaceutically acceptable salt thereof per day. In some embodiments, the method comprises administering to the patient about 100 mg to about 200 mg of the compound of Formula (I) or pharmaceutically acceptable salt thereof per day. In some embodiments, the method comprises administering to the patient about 100 mg to about 150 mg of the compound of Formula (I) or pharmaceutically acceptable salt thereof per day.

In some embodiments, the method comprises administering to the patient about 50 mg of the compound of Formula (I) or pharmaceutically acceptable salt thereof per day. In some embodiments, the method comprises administering to the patient about 55 mg of the compound of Formula (I) or pharmaceutically acceptable salt thereof per day. In some embodiments, the method comprises administering to the patient about 60 mg of the compound of Formula (I) or pharmaceutically acceptable salt thereof per day. In some embodiments, the method comprises administering to the patient about 65 mg of the compound of Formula (I) or pharmaceutically acceptable salt thereof per day. In some embodiments, the method comprises administering to the patient about 70 mg of the compound of Formula (I) or pharmaceutically acceptable salt thereof per day. In some embodiments, the method comprises administering to the patient about 75 mg of the compound of Formula (I) or pharmaceutically acceptable salt thereof per day. In some embodiments, the method comprises administering to the patient about 80 mg of the compound of Formula (I) or pharmaceutically acceptable salt thereof per day. In some embodiments, the method comprises administering to the patient about 85 mg of the compound of Formula (I) or pharmaceutically acceptable salt thereof per day. In some embodiments, the method comprises administering to the patient about 90 mg of the compound of Formula (I) or pharmaceutically acceptable salt thereof per day. In some embodiments, the method comprises administering to the patient about 95 mg of the compound of Formula (I) or pharmaceutically acceptable salt thereof per day. In some embodiments, the method comprises administering to the patient about 100 mg of the compound of Formula (I) or pharmaceutically acceptable salt thereof per day. In some embodiments, the method comprises administering to the patient about 105 mg of the compound of Formula (I) or pharmaceutically acceptable salt thereof per day. In some embodiments, the method comprises administering to the patient about 110 mg of the compound of Formula (I) or pharmaceutically acceptable salt thereof per day. In some embodiments, the method comprises administering to the patient about 115 mg of the compound of Formula (I) or pharmaceutically acceptable salt thereof per day. In some embodiments, the method comprises administering to the patient about 120 mg of the compound of Formula (I) or pharmaceutically acceptable salt thereof per day. In some embodiments, the method comprises administering to the patient about 125 mg of the compound of Formula (I) or pharmaceutically acceptable salt thereof per day. In some embodiments, the method comprises administering to the patient about 130 mg of the compound of Formula (I) or pharmaceutically acceptable salt thereof per day. In some embodiments, the method comprises administering to the patient about 135 mg of the compound of Formula (I) or pharmaceutically acceptable salt thereof per day. In some embodiments, the method comprises administering to the patient about 140 mg of the compound of Formula (I) or pharmaceutically acceptable salt thereof per day. In some embodiments, the method comprises administering to the patient about 145 mg of the compound of Formula (I) or pharmaceutically acceptable salt thereof per day. In some embodiments, the method comprises administering to the patient about 150 mg of the compound of Formula (I) or pharmaceutically acceptable salt thereof per day. In some embodiments, the method comprises administering to the patient about 155 mg of the compound of Formula (I) or pharmaceutically acceptable salt thereof per day. In some embodiments, the method comprises administering to the patient about 160 mg of the compound of Formula (I) or pharmaceutically acceptable salt thereof per day. In some embodiments, the method comprises administering to the patient about 165 mg of the compound of Formula (I) or pharmaceutically acceptable salt thereof per day. In some embodiments, the method comprises administering to the patient about 170 mg of the compound of Formula (I) or pharmaceutically acceptable salt thereof per day. In some embodiments, the method comprises administering to the patient about 175 mg of the compound of Formula (I) or pharmaceutically acceptable salt thereof per day. In some embodiments, the method comprises administering to the patient about 180 mg of the compound of Formula (I) or pharmaceutically acceptable salt thereof per day. In some embodiments, the method comprises administering to the patient about 185 mg of the compound of Formula (I) or pharmaceutically acceptable salt thereof per day. In some embodiments, the method comprises administering to the patient about 190 mg of the compound of Formula (I) or pharmaceutically acceptable salt thereof per day. In some embodiments, the method comprises administering to the patient about 195 mg of the compound of Formula (I) or pharmaceutically acceptable salt thereof per day. In some embodiments, the method comprises administering to the patient about 200 mg of the compound of Formula (I) or pharmaceutically acceptable salt thereof per day.

In some embodiments, the method comprises administering to the patient about 50 mg to about 200 mg of the compound of Formula (I) or pharmaceutically acceptable salt thereof once per day. In some embodiments, the method comprises administering to the patient about 50 mg to about 150 mg of the compound of Formula (I) or pharmaceutically acceptable salt thereof once per day. In some embodiments, the method comprises administering to the patient about 50 mg to about 100 mg of the compound of Formula (I) or pharmaceutically acceptable salt thereof once per day. In some embodiments, the method comprises administering to the patient about 60 mg to about 200 mg of the compound of Formula (I) or pharmaceutically acceptable salt thereof once per day. In some embodiments, the method comprises administering to the patient about 60 mg to about 150 mg of the compound of Formula (I) or pharmaceutically acceptable salt thereof once per day. In some embodiments, the method comprises administering to the patient about 60 mg to about 100 mg of the compound of Formula (I) or pharmaceutically acceptable salt thereof once per day. In some embodiments, the method comprises administering to the patient about 70 mg to about 200 mg of the compound of Formula (I) or pharmaceutically acceptable salt thereof once per day. In some embodiments, the method comprises administering to the patient about 70 mg to about 150 mg of the compound of Formula (I) or pharmaceutically acceptable salt thereof once per day. In some embodiments, the method comprises administering to the patient about 70 mg to about 100 mg of the compound of Formula (I) or pharmaceutically acceptable salt thereof once per day. In some embodiments, the method comprises administering to the patient about 80 mg to about 200 mg of the compound of Formula (I) or pharmaceutically acceptable salt thereof once per day. In some embodiments, the method comprises administering to the patient about 80 mg to about 150 mg of the compound of Formula (I) or pharmaceutically acceptable salt thereof once per day. In some embodiments, the method comprises administering to the patient about 80 mg to about 100 mg of the compound of Formula (I) or pharmaceutically acceptable salt thereof once per day. In some embodiments, the method comprises administering to the patient about 90 mg to about 200 mg of the compound of Formula (I) or pharmaceutically acceptable salt thereof once per day. In some embodiments, the method comprises administering to the patient about 90 mg to about 150 mg of the compound of Formula (I) or pharmaceutically acceptable salt thereof once per day. In some embodiments, the method comprises administering to the patient about 90 mg to about 100 mg of the compound of Formula (I) or pharmaceutically acceptable salt thereof once per day. In some embodiments, the method comprises administering to the patient about 100 mg to about 200 mg of the compound of Formula (I) or pharmaceutically acceptable salt thereof once per day. In some embodiments, the method comprises administering to the patient about 100 mg to about 150 mg of the compound of Formula (I) or pharmaceutically acceptable salt thereof once per day.

In some embodiments, the method comprises administering to the patient about 50 mg of the compound of Formula (I) or pharmaceutically acceptable salt thereof once per day. In some embodiments, the method comprises administering to the patient about 55 mg of the compound of Formula (I) or pharmaceutically acceptable salt thereof once per day. In some embodiments, the method comprises administering to the patient about 60 mg of the compound of Formula (I) or pharmaceutically acceptable salt thereof once per day. In some embodiments, the method comprises administering to the patient about 65 mg of the compound of Formula (I) or pharmaceutically acceptable salt thereof once per day. In some embodiments, the method comprises administering to the patient about 70 mg of the compound of Formula (I) or pharmaceutically acceptable salt thereof once per day. In some embodiments, the method comprises administering to the patient about 75 mg of the compound of Formula (I) or pharmaceutically acceptable salt thereof once per day. In some embodiments, the method comprises administering to the patient about 80 mg of the compound of Formula (I) or pharmaceutically acceptable salt thereof once per day. In some embodiments, the method comprises administering to the patient about 85 mg of the compound of Formula (I) or pharmaceutically acceptable salt thereof once per day. In some embodiments, the method comprises administering to the patient about 90 mg of the compound of Formula (I) or pharmaceutically acceptable salt thereof once per day. In some embodiments, the method comprises administering to the patient about 95 mg of the compound of Formula (I) or pharmaceutically acceptable salt thereof once per day. In some embodiments, the method comprises administering to the patient about 100 mg of the compound of Formula (I) or pharmaceutically acceptable salt thereof once per day. In some embodiments, the method comprises administering to the patient about 105 mg of the compound of Formula (I) or pharmaceutically acceptable salt thereof once per day. In some embodiments, the method comprises administering to the patient about 110 mg of the compound of Formula (I) or pharmaceutically acceptable salt thereof once per day. In some embodiments, the method comprises administering to the patient about 115 mg of the compound of Formula (I) or pharmaceutically acceptable salt thereof once per day. In some embodiments, the method comprises administering to the patient about 120 mg of the compound of Formula (I) or pharmaceutically acceptable salt thereof once per day. In some embodiments, the method comprises administering to the patient about 125 mg of the compound of Formula (I) or pharmaceutically acceptable salt thereof once per day. In some embodiments, the method comprises administering to the patient about 130 mg of the compound of Formula (I) or pharmaceutically acceptable salt thereof once per day. In some embodiments, the method comprises administering to the patient about 135 mg of the compound of Formula (I) or pharmaceutically acceptable salt thereof once per day. In some embodiments, the method comprises administering to the patient about 140 mg of the compound of Formula (I) or pharmaceutically acceptable salt thereof once per day. In some embodiments, the method comprises administering to the patient about 145 mg of the compound of Formula (I) or pharmaceutically acceptable salt thereof once per day. In some embodiments, the method comprises administering to the patient about 150 mg of the compound of Formula (I) or pharmaceutically acceptable salt thereof once per day. In some embodiments, the method comprises administering to the patient about 155 mg of the compound of Formula (I) or pharmaceutically acceptable salt thereof once per day. In some embodiments, the method comprises administering to the patient about 160 mg of the compound of Formula (I) or pharmaceutically acceptable salt thereof once per day. In some embodiments, the method comprises administering to the patient about 165 mg of the compound of Formula (I) or pharmaceutically acceptable salt thereof once per day. In some embodiments, the method comprises administering to the patient about 170 mg of the compound of Formula (I) or pharmaceutically acceptable salt thereof once per day. In some embodiments, the method comprises administering to the patient about 175 mg of the compound of Formula (I) or pharmaceutically acceptable salt thereof once per day. In some embodiments, the method comprises administering to the patient about 180 mg of the compound of Formula (I) or pharmaceutically acceptable salt thereof once per day. In some embodiments, the method comprises administering to the patient about 185 mg of the compound of Formula (I) or pharmaceutically acceptable salt thereof once per day. In some embodiments, the method comprises administering to the patient about 190 mg of the compound of Formula (I) or pharmaceutically acceptable salt thereof once per day. In some embodiments, the method comprises administering to the patient about 195 mg of the compound of Formula (I) or pharmaceutically acceptable salt thereof once per day. In some embodiments, the method comprises administering to the patient about 200 mg of the compound of Formula (I) or pharmaceutically acceptable salt thereof once per day.

In some embodiments, the method comprises administering to the patient about 50 mg to about 100 mg of the compound of Formula (I) or pharmaceutically acceptable salt thereof twice per day. In some embodiments, the method comprises administering to the patient about 60 mg to about 100 mg of the compound of Formula (I) or pharmaceutically acceptable salt thereof twice per day. In some embodiments, the method comprises administering to the patient about 70 mg to about 100 mg of the compound of Formula (I) or pharmaceutically acceptable salt thereof twice per day. In some embodiments, the method comprises administering to the patient about 80 mg to about 100 mg of the compound of Formula (I) or pharmaceutically acceptable salt thereof twice per day. In some embodiments, the method comprises administering to the patient about 90 mg to about 100 mg of the compound of Formula (I) or pharmaceutically acceptable salt thereof twice per day.

In some embodiments, the method comprises administering to the patient about 50 mg of the compound of Formula (I) or pharmaceutically acceptable salt thereof twice per day. In some embodiments, the method comprises administering to the patient about 55 mg of the compound of Formula (I) or pharmaceutically acceptable salt thereof twice per day. In some embodiments, the method comprises administering to the patient about 60 mg of the compound of Formula (I) or pharmaceutically acceptable salt thereof twice per day. In some embodiments, the method comprises administering to the patient about 65 mg of the compound of Formula (I) or pharmaceutically acceptable salt thereof twice per day. In some embodiments, the method comprises administering to the patient about 70 mg of the compound of Formula (I) or pharmaceutically acceptable salt thereof twice per day. In some embodiments, the method comprises administering to the patient about 75 mg of the compound of Formula (I) or pharmaceutically acceptable salt thereof twice per day. In some embodiments, the method comprises administering to the patient about 80 mg of the compound of Formula (I) or pharmaceutically acceptable salt thereof twice per day. In some embodiments, the method comprises administering to the patient about 85 mg of the compound of Formula (I) or pharmaceutically acceptable salt thereof twice per day. In some embodiments, the method comprises administering to the patient about 90 mg of the compound of Formula (I) or pharmaceutically acceptable salt thereof twice per day. In some embodiments, the method comprises administering to the patient about 95 mg of the compound of Formula (I) or pharmaceutically acceptable salt thereof twice per day. In some embodiments, the method comprises administering to the patient about 100 mg of the compound of Formula (I) or pharmaceutically acceptable salt thereof twice per day.

The compound of Formula (I) or pharmaceutically acceptable salt thereof may, in another embodiment, be administered to the patient on an intermittent basis in the methods of achieving a stable disease condition in a patient having endometrial adenocarcinoma described herein. In some embodiments, the compound of Formula (I) or pharmaceutically acceptable salt thereof is administered the patient every other day. In some embodiments, the compound of Formula (I) or pharmaceutically acceptable salt thereof is administered the patient every other day. In some embodiments, the compound of Formula (I) or pharmaceutically acceptable salt thereof is administered the patient once per week. In some embodiments, the compound of Formula (I) or pharmaceutically acceptable salt thereof is administered the patient twice per week. In some embodiments, the compound of Formula (I) or pharmaceutically acceptable salt thereof is administered the patient three times per week.

In some embodiments, the method comprises administering to the patient about 50 mg to about 200 mg of the compound of Formula (I) or pharmaceutically acceptable salt thereof once a week. In some embodiments, the method comprises administering to the patient about 50 mg to about 150 mg of the compound of Formula (I) or pharmaceutically acceptable salt thereof once a week. In some embodiments, the method comprises administering to the patient about 50 mg to about 100 mg of the compound of Formula (I) or pharmaceutically acceptable salt thereof once a week. In some embodiments, the method comprises administering to the patient about 60 mg to about 200 mg of the compound of Formula (I) or pharmaceutically acceptable salt thereof once a week. In some embodiments, the method comprises administering to the patient about 60 mg to about 150 mg of the compound of Formula (I) or pharmaceutically acceptable salt thereof once a week. In some embodiments, the method comprises administering to the patient about 60 mg to about 100 mg of the compound of Formula (I) or pharmaceutically acceptable salt thereof once a week. In some embodiments, the method comprises administering to the patient about 70 mg to about 200 mg of the compound of Formula (I) or pharmaceutically acceptable salt thereof once a week. In some embodiments, the method comprises administering to the patient about 70 mg to about 150 mg of the compound of Formula (I) or pharmaceutically acceptable salt thereof once a week. In some embodiments, the method comprises administering to the patient about 70 mg to about 100 mg of the compound of Formula (I) or pharmaceutically acceptable salt thereof once a week. In some embodiments, the method comprises administering to the patient about 80 mg to about 200 mg of the compound of Formula (I) or pharmaceutically acceptable salt thereof once a week. In some embodiments, the method comprises administering to the patient about 80 mg to about 150 mg of the compound of Formula (I) or pharmaceutically acceptable salt thereof once a week. In some embodiments, the method comprises administering to the patient about 80 mg to about 100 mg of the compound of Formula (I) or pharmaceutically acceptable salt thereof once a week. In some embodiments, the method comprises administering to the patient about 90 mg to about 200 mg of the compound of Formula (I) or pharmaceutically acceptable salt thereof once a week. In some embodiments, the method comprises administering to the patient about 90 mg to about 150 mg of the compound of Formula (I) or pharmaceutically acceptable salt thereof once a week. In some embodiments, the method comprises administering to the patient about 90 mg to about 100 mg of the compound of Formula (I) or pharmaceutically acceptable salt thereof once a week. In some embodiments, the method comprises administering to the patient about 100 mg to about 200 mg of the compound of Formula (I) or pharmaceutically acceptable salt thereof once a week. In some embodiments, the method comprises administering to the patient about 100 mg to about 150 mg of the compound of Formula (I) or pharmaceutically acceptable salt thereof once a week.

In some embodiments, the method comprises administering to the patient about 50 mg of the compound of Formula (I) or pharmaceutically acceptable salt thereof once a week. In some embodiments, the method comprises administering to the patient about 55 mg of the compound of Formula (I) or pharmaceutically acceptable salt thereof once a week. In some embodiments, the method comprises administering to the patient about 60 mg of the compound of Formula (I) or pharmaceutically acceptable salt thereof once a week. In some embodiments, the method comprises administering to the patient about 65 mg of the compound of Formula (I) or pharmaceutically acceptable salt thereof once a week. In some embodiments, the method comprises administering to the patient about 70 mg of the compound of Formula (I) or pharmaceutically acceptable salt thereof once a week. In some embodiments, the method comprises administering to the patient about 75 mg of the compound of Formula (I) or pharmaceutically acceptable salt thereof once a week. In some embodiments, the method comprises administering to the patient about 80 mg of the compound of Formula (I) or pharmaceutically acceptable salt thereof once a week. In some embodiments, the method comprises administering to the patient about 85 mg of the compound of Formula (I) or pharmaceutically acceptable salt thereof once a week. In some embodiments, the method comprises administering to the patient about 90 mg of the compound of Formula (I) or pharmaceutically acceptable salt thereof once a week. In some embodiments, the method comprises administering to the patient about 95 mg of the compound of Formula (I) or pharmaceutically acceptable salt thereof once a week. In some embodiments, the method comprises administering to the patient about 100 mg of the compound of Formula (I) or pharmaceutically acceptable salt thereof once a week. In some embodiments, the method comprises administering to the patient about 105 mg of the compound of Formula (I) or pharmaceutically acceptable salt thereof once a week. In some embodiments, the method comprises administering to the patient about 110 mg of the compound of Formula (I) or pharmaceutically acceptable salt thereof once a week. In some embodiments, the method comprises administering to the patient about 115 mg of the compound of Formula (I) or pharmaceutically acceptable salt thereof once a week. In some embodiments, the method comprises administering to the patient about 120 mg of the compound of Formula (I) or pharmaceutically acceptable salt thereof once a week. In some embodiments, the method comprises administering to the patient about 125 mg of the compound of Formula (I) or pharmaceutically acceptable salt thereof once a week. In some embodiments, the method comprises administering to the patient about 130 mg of the compound of Formula (I) or pharmaceutically acceptable salt thereof once a week. In some embodiments, the method comprises administering to the patient about 135 mg of the compound of Formula (I) or pharmaceutically acceptable salt thereof once a week. In some embodiments, the method comprises administering to the patient about 140 mg of the compound of Formula (I) or pharmaceutically acceptable salt thereof once a week. In some embodiments, the method comprises administering to the patient about 145 mg of the compound of Formula (I) or pharmaceutically acceptable salt thereof once a week. In some embodiments, the method comprises administering to the patient about 150 mg of the compound of Formula (I) or pharmaceutically acceptable salt thereof once a week. In some embodiments, the method comprises administering to the patient about 155 mg of the compound of Formula (I) or pharmaceutically acceptable salt thereof once a week. In some embodiments, the method comprises administering to the patient about 160 mg of the compound of Formula (I) or pharmaceutically acceptable salt thereof once a week. In some embodiments, the method comprises administering to the patient about 165 mg of the compound of Formula (I) or pharmaceutically acceptable salt thereof once a week. In some embodiments, the method comprises administering to the patient about 170 mg of the compound of Formula (I) or pharmaceutically acceptable salt thereof once a week. In some embodiments, the method comprises administering to the patient about 175 mg of the compound of Formula (I) or pharmaceutically acceptable salt thereof once a week. In some embodiments, the method comprises administering to the patient about 180 mg of the compound of Formula (I) or pharmaceutically acceptable salt thereof once a week. In some embodiments, the method comprises administering to the patient about 185 mg of the compound of Formula (I) or pharmaceutically acceptable salt thereof once a week. In some embodiments, the method comprises administering to the patient about 190 mg of the compound of Formula (I) or pharmaceutically acceptable salt thereof once a week. In some embodiments, the method comprises administering to the patient about 195 mg of the compound of Formula (I) or pharmaceutically acceptable salt thereof once a week. In some embodiments, the method comprises administering to the patient about 200 mg of the compound of Formula (I) or pharmaceutically acceptable salt thereof once a week.

In some embodiments, the method comprises administering to the patient about 50 mg to about 200 mg of the compound of Formula (I) or pharmaceutically acceptable salt thereof twice a week. In some embodiments, the method comprises administering to the patient about 50 mg to about 150 mg of the compound of Formula (I) or pharmaceutically acceptable salt thereof twice a week. In some embodiments, the method comprises administering to the patient about 50 mg to about 100 mg of the compound of Formula (I) or pharmaceutically acceptable salt thereof twice a week. In some embodiments, the method comprises administering to the patient about 60 mg to about 200 mg of the compound of Formula (I) or pharmaceutically acceptable salt thereof twice a week. In some embodiments, the method comprises administering to the patient about 60 mg to about 150 mg of the compound of Formula (I) or pharmaceutically acceptable salt thereof twice a week. In some embodiments, the method comprises administering to the patient about 60 mg to about 100 mg of the compound of Formula (I) or pharmaceutically acceptable salt thereof twice a week. In some embodiments, the method comprises administering to the patient about 70 mg to about 200 mg of the compound of Formula (I) or pharmaceutically acceptable salt thereof twice a week. In some embodiments, the method comprises administering to the patient about 70 mg to about 150 mg of the compound of Formula (I) or pharmaceutically acceptable salt thereof twice a week. In some embodiments, the method comprises administering to the patient about 70 mg to about 100 mg of the compound of Formula (I) or pharmaceutically acceptable salt thereof twice a week. In some embodiments, the method comprises administering to the patient about 80 mg to about 200 mg of the compound of Formula (I) or pharmaceutically acceptable salt thereof twice a week. In some embodiments, the method comprises administering to the patient about 80 mg to about 150 mg of the compound of Formula (I) or pharmaceutically acceptable salt thereof twice a week. In some embodiments, the method comprises administering to the patient about 80 mg to about 100 mg of the compound of Formula (I) or pharmaceutically acceptable salt thereof twice a week. In some embodiments, the method comprises administering to the patient about 90 mg to about 200 mg of the compound of Formula (I) or pharmaceutically acceptable salt thereof twice a week. In some embodiments, the method comprises administering to the patient about 90 mg to about 150 mg of the compound of Formula (I) or pharmaceutically acceptable salt thereof twice a week. In some embodiments, the method comprises administering to the patient about 90 mg to about 100 mg of the compound of Formula (I) or pharmaceutically acceptable salt thereof twice a week. In some embodiments, the method comprises administering to the patient about 100 mg to about 200 mg of the compound of Formula (I) or pharmaceutically acceptable salt thereof twice a week. In some embodiments, the method comprises administering to the patient about 100 mg to about 150 mg of the compound of Formula (I) or pharmaceutically acceptable salt thereof twice a week.

In some embodiments, the method comprises administering to the patient about 50 mg of the compound of Formula (I) or pharmaceutically acceptable salt thereof twice a week. In some embodiments, the method comprises administering to the patient about 55 mg of the compound of Formula (I) or pharmaceutically acceptable salt thereof twice a week. In some embodiments, the method comprises administering to the patient about 60 mg of the compound of Formula (I) or pharmaceutically acceptable salt thereof twice a week. In some embodiments, the method comprises administering to the patient about 65 mg of the compound of Formula (I) or pharmaceutically acceptable salt thereof twice a week. In some embodiments, the method comprises administering to the patient about 70 mg of the compound of Formula (I) or pharmaceutically acceptable salt thereof twice a week. In some embodiments, the method comprises administering to the patient about 75 mg of the compound of Formula (I) or pharmaceutically acceptable salt thereof twice a week. In some embodiments, the method comprises administering to the patient about 80 mg of the compound of Formula (I) or pharmaceutically acceptable salt thereof twice a week. In some embodiments, the method comprises administering to the patient about 85 mg of the compound of Formula (I) or pharmaceutically acceptable salt thereof twice a week. In some embodiments, the method comprises administering to the patient about 90 mg of the compound of Formula (I) or pharmaceutically acceptable salt thereof twice a week. In some embodiments, the method comprises administering to the patient about 95 mg of the compound of Formula (I) or pharmaceutically acceptable salt thereof twice a week. In some embodiments, the method comprises administering to the patient about 100 mg of the compound of Formula (I) or pharmaceutically acceptable salt thereof twice a week. In some embodiments, the method comprises administering to the patient about 105 mg of the compound of Formula (I) or pharmaceutically acceptable salt thereof twice a week. In some embodiments, the method comprises administering to the patient about 110 mg of the compound of Formula (I) or pharmaceutically acceptable salt thereof twice a week. In some embodiments, the method comprises administering to the patient about 115 mg of the compound of Formula (I) or pharmaceutically acceptable salt thereof twice a week. In some embodiments, the method comprises administering to the patient about 120 mg of the compound of Formula (I) or pharmaceutically acceptable salt thereof twice a week. In some embodiments, the method comprises administering to the patient about 125 mg of the compound of Formula (I) or pharmaceutically acceptable salt thereof twice a week. In some embodiments, the method comprises administering to the patient about 130 mg of the compound of Formula (I) or pharmaceutically acceptable salt thereof twice a week. In some embodiments, the method comprises administering to the patient about 135 mg of the compound of Formula (I) or pharmaceutically acceptable salt thereof twice a week. In some embodiments, the method comprises administering to the patient about 140 mg of the compound of Formula (I) or pharmaceutically acceptable salt thereof twice a week. In some embodiments, the method comprises administering to the patient about 145 mg of the compound of Formula (I) or pharmaceutically acceptable salt thereof twice a week. In some embodiments, the method comprises administering to the patient about 150 mg of the compound of Formula (I) or pharmaceutically acceptable salt thereof twice a week. In some embodiments, the method comprises administering to the patient about 155 mg of the compound of Formula (I) or pharmaceutically acceptable salt thereof twice a week. In some embodiments, the method comprises administering to the patient about 160 mg of the compound of Formula (I) or pharmaceutically acceptable salt thereof twice a week. In some embodiments, the method comprises administering to the patient about 165 mg of the compound of Formula (I) or pharmaceutically acceptable salt thereof twice a week. In some embodiments, the method comprises administering to the patient about 170 mg of the compound of Formula (I) or pharmaceutically acceptable salt thereof twice a week. In some embodiments, the method comprises administering to the patient about 175 mg of the compound of Formula (I) or pharmaceutically acceptable salt thereof twice a week. In some embodiments, the method comprises administering to the patient about 180 mg of the compound of Formula (I) or pharmaceutically acceptable salt thereof twice a week. In some embodiments, the method comprises administering to the patient about 185 mg of the compound of Formula (I) or pharmaceutically acceptable salt thereof twice a week. In some embodiments, the method comprises administering to the patient about 190 mg of the compound of Formula (I) or pharmaceutically acceptable salt thereof twice a week. In some embodiments, the method comprises administering to the patient about 195 mg of the compound of Formula (I) or pharmaceutically acceptable salt thereof twice a week. In some embodiments, the method comprises administering to the patient about 200 mg of the compound of Formula (I) or pharmaceutically acceptable salt thereof twice a week.

In some embodiments, the method comprises administering to the patient about 50 mg to about 200 mg of the compound of Formula (I) or pharmaceutically acceptable salt thereof three times a week. In some embodiments, the method comprises administering to the patient about 50 mg to about 150 mg of the compound of Formula (I) or pharmaceutically acceptable salt thereof three times a week. In some embodiments, the method comprises administering to the patient about 50 mg to about 100 mg of the compound of Formula (I) or pharmaceutically acceptable salt thereof three times a week. In some embodiments, the method comprises administering to the patient about 60 mg to about 200 mg of the compound of Formula (I) or pharmaceutically acceptable salt thereof three times a week. In some embodiments, the method comprises administering to the patient about 60 mg to about 150 mg of the compound of Formula (I) or pharmaceutically acceptable salt thereof three times a week. In some embodiments, the method comprises administering to the patient about 60 mg to about 100 mg of the compound of Formula (I) or pharmaceutically acceptable salt thereof three times a week. In some embodiments, the method comprises administering to the patient about 70 mg to about 200 mg of the compound of Formula (I) or pharmaceutically acceptable salt thereof three times a week. In some embodiments, the method comprises administering to the patient about 70 mg to about 150 mg of the compound of Formula (I) or pharmaceutically acceptable salt thereof three times a week. In some embodiments, the method comprises administering to the patient about 70 mg to about 100 mg of the compound of Formula (I) or pharmaceutically acceptable salt thereof three times a week. In some embodiments, the method comprises administering to the patient about 80 mg to about 200 mg of the compound of Formula (I) or pharmaceutically acceptable salt thereof three times a week. In some embodiments, the method comprises administering to the patient about 80 mg to about 150 mg of the compound of Formula (I) or pharmaceutically acceptable salt thereof three times a week. In some embodiments, the method comprises administering to the patient about 80 mg to about 100 mg of the compound of Formula (I) or pharmaceutically acceptable salt thereof three times a week. In some embodiments, the method comprises administering to the patient about 90 mg to about 200 mg of the compound of Formula (I) or pharmaceutically acceptable salt thereof three times a week. In some embodiments, the method comprises administering to the patient about 90 mg to about 150 mg of the compound of Formula (I) or pharmaceutically acceptable salt thereof three times a week. In some embodiments, the method comprises administering to the patient about 90 mg to about 100 mg of the compound of Formula (I) or pharmaceutically acceptable salt thereof three times a week. In some embodiments, the method comprises administering to the patient about 100 mg to about 200 mg of the compound of Formula (I) or pharmaceutically acceptable salt thereof three times a week. In some embodiments, the method comprises administering to the patient about 100 mg to about 150 mg of the compound of Formula (I) or pharmaceutically acceptable salt thereof three times a week.

In some embodiments, the method comprises administering to the patient about 50 mg of the compound of Formula (I) or pharmaceutically acceptable salt thereof three times a week. In some embodiments, the method comprises administering to the patient about 55 mg of the compound of Formula (I) or pharmaceutically acceptable salt thereof three times a week. In some embodiments, the method comprises administering to the patient about 60 mg of the compound of Formula (I) or pharmaceutically acceptable salt thereof three times a week. In some embodiments, the method comprises administering to the patient about 65 mg of the compound of Formula (I) or pharmaceutically acceptable salt thereof three times a week. In some embodiments, the method comprises administering to the patient about 70 mg of the compound of Formula (I) or pharmaceutically acceptable salt thereof three times a week. In some embodiments, the method comprises administering to the patient about 75 mg of the compound of Formula (I) or pharmaceutically acceptable salt thereof three times a week. In some embodiments, the method comprises administering to the patient about 80 mg of the compound of Formula (I) or pharmaceutically acceptable salt thereof three times a week. In some embodiments, the method comprises administering to the patient about 85 mg of the compound of Formula (I) or pharmaceutically acceptable salt thereof three times a week. In some embodiments, the method comprises administering to the patient about 90 mg of the compound of Formula (I) or pharmaceutically acceptable salt thereof three times a week. In some embodiments, the method comprises administering to the patient about 95 mg of the compound of Formula (I) or pharmaceutically acceptable salt thereof three times a week. In some embodiments, the method comprises administering to the patient about 100 mg of the compound of Formula (I) or pharmaceutically acceptable salt thereof three times a week. In some embodiments, the method comprises administering to the patient about 105 mg of the compound of Formula (I) or pharmaceutically acceptable salt thereof three times a week. In some embodiments, the method comprises administering to the patient about 110 mg of the compound of Formula (I) or pharmaceutically acceptable salt thereof three times a week. In some embodiments, the method comprises administering to the patient about 115 mg of the compound of Formula (I) or pharmaceutically acceptable salt thereof three times a week. In some embodiments, the method comprises administering to the patient about 120 mg of the compound of Formula (I) or pharmaceutically acceptable salt thereof three times a week. In some embodiments, the method comprises administering to the patient about 125 mg of the compound of Formula (I) or pharmaceutically acceptable salt thereof three times a week. In some embodiments, the method comprises administering to the patient about 130 mg of the compound of Formula (I) or pharmaceutically acceptable salt thereof three times a week. In some embodiments, the method comprises administering to the patient about 135 mg of the compound of Formula (I) or pharmaceutically acceptable salt thereof three times a week. In some embodiments, the method comprises administering to the patient about 140 mg of the compound of Formula (I) or pharmaceutically acceptable salt thereof three times a week. In some embodiments, the method comprises administering to the patient about 145 mg of the compound of Formula (I) or pharmaceutically acceptable salt thereof three times a week. In some embodiments, the method comprises administering to the patient about 150 mg of the compound of Formula (I) or pharmaceutically acceptable salt thereof three times a week. In some embodiments, the method comprises administering to the patient about 155 mg of the compound of Formula (I) or pharmaceutically acceptable salt thereof three times a week. In some embodiments, the method comprises administering to the patient about 160 mg of the compound of Formula (I) or pharmaceutically acceptable salt thereof three times a week. In some embodiments, the method comprises administering to the patient about 165 mg of the compound of Formula (I) or pharmaceutically acceptable salt thereof three times a week. In some embodiments, the method comprises administering to the patient about 170 mg of the compound of Formula (I) or pharmaceutically acceptable salt thereof three times a week. In some embodiments, the method comprises administering to the patient about 175 mg of the compound of Formula (I) or pharmaceutically acceptable salt thereof three times a week. In some embodiments, the method comprises administering to the patient about 180 mg of the compound of Formula (I) or pharmaceutically acceptable salt thereof three times a week. In some embodiments, the method comprises administering to the patient about 185 mg of the compound of Formula (I) or pharmaceutically acceptable salt thereof three times a week. In some embodiments, the method comprises administering to the patient about 190 mg of the compound of Formula (I) or pharmaceutically acceptable salt thereof three times a week. In some embodiments, the method comprises administering to the patient about 195 mg of the compound of Formula (I) or pharmaceutically acceptable salt thereof three times a week. In some embodiments, the method comprises administering to the patient about 200 mg of the compound of Formula (I) or pharmaceutically acceptable salt thereof three times a week.

In some embodiments, the method comprises administering to the patient about 50 mg to about 200 mg of the compound of Formula (I) or pharmaceutically acceptable salt thereof every other day. In some embodiments, the method comprises administering to the patient about 50 mg to about 150 mg of the compound of Formula (I) or pharmaceutically acceptable salt thereof every other day. In some embodiments, the method comprises administering to the patient about 50 mg to about 100 mg of the compound of Formula (I) or pharmaceutically acceptable salt thereof every other day. In some embodiments, the method comprises administering to the patient about 60 mg to about 200 mg of the compound of Formula (I) or pharmaceutically acceptable salt thereof every other day. In some embodiments, the method comprises administering to the patient about 60 mg to about 150 mg of the compound of Formula (I) or pharmaceutically acceptable salt thereof every other day. In some embodiments, the method comprises administering to the patient about 60 mg to about 100 mg of the compound of Formula (I) or pharmaceutically acceptable salt thereof every other day. In some embodiments, the method comprises administering to the patient about 70 mg to about 200 mg of the compound of Formula (I) or pharmaceutically acceptable salt thereof every other day. In some embodiments, the method comprises administering to the patient about 70 mg to about 150 mg of the compound of Formula (I) or pharmaceutically acceptable salt thereof every other day. In some embodiments, the method comprises administering to the patient about 70 mg to about 100 mg of the compound of Formula (I) or pharmaceutically acceptable salt thereof every other day. In some embodiments, the method comprises administering to the patient about 80 mg to about 200 mg of the compound of Formula (I) or pharmaceutically acceptable salt thereof every other day. In some embodiments, the method comprises administering to the patient about 80 mg to about 150 mg of the compound of Formula (I) or pharmaceutically acceptable salt thereof every other day. In some embodiments, the method comprises administering to the patient about 80 mg to about 100 mg of the compound of Formula (I) or pharmaceutically acceptable salt thereof every other day. In some embodiments, the method comprises administering to the patient about 90 mg to about 200 mg of the compound of Formula (I) or pharmaceutically acceptable salt thereof every other day. In some embodiments, the method comprises administering to the patient about 90 mg to about 150 mg of the compound of Formula (I) or pharmaceutically acceptable salt thereof every other day. In some embodiments, the method comprises administering to the patient about 90 mg to about 100 mg of the compound of Formula (I) or pharmaceutically acceptable salt thereof every other day. In some embodiments, the method comprises administering to the patient about 100 mg to about 200 mg of the compound of Formula (I) or pharmaceutically acceptable salt thereof every other day. In some embodiments, the method comprises administering to the patient about 100 mg to about 150 mg of the compound of Formula (I) or pharmaceutically acceptable salt thereof every other day.

In some embodiments, the method comprises administering to the patient about 50 mg of the compound of Formula (I) or pharmaceutically acceptable salt thereof every other day. In some embodiments, the method comprises administering to the patient about 55 mg of the compound of Formula (I) or pharmaceutically acceptable salt thereof every other day. In some embodiments, the method comprises administering to the patient about 60 mg of the compound of Formula (I) or pharmaceutically acceptable salt thereof every other day. In some embodiments, the method comprises administering to the patient about 65 mg of the compound of Formula (I) or pharmaceutically acceptable salt thereof every other day. In some embodiments, the method comprises administering to the patient about 70 mg of the compound of Formula (I) or pharmaceutically acceptable salt thereof every other day. In some embodiments, the method comprises administering to the patient about 75 mg of the compound of Formula (I) or pharmaceutically acceptable salt thereof every other day. In some embodiments, the method comprises administering to the patient about 80 mg of the compound of Formula (I) or pharmaceutically acceptable salt thereof every other day. In some embodiments, the method comprises administering to the patient about 85 mg of the compound embodiments, the method comprises administering to the patient about 90 mg of the compound of Formula (I) or pharmaceutically acceptable salt thereof every other day. In some embodiments, the method comprises administering to the patient about 95 mg of the compound of Formula (I) or pharmaceutically acceptable salt thereof every other day. In some embodiments, the method comprises administering to the patient about 100 mg of the compound of Formula (I) or pharmaceutically acceptable salt thereof every other day. In some embodiments, the method comprises administering to the patient about 105 mg of the compound of Formula (I) or pharmaceutically acceptable salt thereof every other day. In some embodiments, the method comprises administering to the patient about 110 mg of the compound of Formula (I) or pharmaceutically acceptable salt thereof every other day. In some embodiments, the method comprises administering to the patient about 115 mg of the compound of Formula (I) or pharmaceutically acceptable salt thereof every other day. In some embodiments, the method comprises administering to the patient about 120 mg of the compound of Formula (I) or pharmaceutically acceptable salt thereof every other day. In some embodiments, the method comprises administering to the patient about 125 mg of the compound of Formula (I) or pharmaceutically acceptable salt thereof every other day. In some embodiments, the method comprises administering to the patient about 130 mg of the compound of Formula (I) or pharmaceutically acceptable salt thereof every other day. In some embodiments, the method comprises administering to the patient about 135 mg of the compound of Formula (I) or pharmaceutically acceptable salt thereof every other day. In some embodiments, the method comprises administering to the patient about 140 mg of the compound of Formula (I) or pharmaceutically acceptable salt thereof every other day. In some embodiments, the method comprises administering to the patient about 145 mg of the compound of Formula (I) or pharmaceutically acceptable salt thereof every other day. In some embodiments, the method comprises administering to the patient about 150 mg of the compound of Formula (I) or pharmaceutically acceptable salt thereof every other day. In some embodiments, the method comprises administering to the patient about 155 mg of the compound of Formula (I) or pharmaceutically acceptable salt thereof every other day. In some embodiments, the method comprises administering to the patient about 160 mg of the compound of Formula (I) or pharmaceutically acceptable salt thereof every other day. In some embodiments, the method comprises administering to the patient about 165 mg of the compound of Formula (I) or pharmaceutically acceptable salt thereof every other day. In some embodiments, the method comprises administering to the patient about 170 mg of the compound of Formula (I) or pharmaceutically acceptable salt thereof every other day. In some embodiments, the method comprises administering to the patient about 175 mg of the compound of Formula (I) or pharmaceutically acceptable salt thereof every other day. In some embodiments, the method comprises administering to the patient about 180 mg of the compound of Formula (I) or pharmaceutically acceptable salt thereof every other day. In some embodiments, the method comprises administering to the patient about 185 mg of the compound of Formula (I) or pharmaceutically acceptable salt thereof every other day. In some embodiments, the method comprises administering to the patient about 190 mg of the compound of Formula (I) or pharmaceutically acceptable salt thereof every other day. In some embodiments, the method comprises administering to the patient about 195 mg of the compound of Formula (I) or pharmaceutically acceptable salt thereof every other day. In some embodiments, the method comprises administering to the patient about 200 mg of the compound of Formula (I) or pharmaceutically acceptable salt thereof every other day.

In some embodiments, a method of achieving a stable disease condition in a patient having an endometrial adenocarcinoma described herein further comprises administering paclitaxel to the patient in need thereof. In some embodiments, the paclitaxel is intravenously administered to the patient, e.g., by intravenous infusion. The intravenously administered paclitaxel can be administered to a patient at various dose levels. In some embodiments, the paclitaxel is administered to the patient at about 50 mg/m² to about 150 mg/m². In some embodiments, the paclitaxel is administered to the patient at about 50 mg/m² to about 100 mg/m². In some embodiments, the paclitaxel is administered to the patient at about 60 mg/m² to about 80 mg/m². In some embodiments, the paclitaxel is administered to the patient at about 60 mg/m². In some embodiments, the paclitaxel is administered to the patient at about 80 mg/m². In some embodiments, the paclitaxel is administered to the patient at about 135 mg/m². In some embodiments, the paclitaxel is administered to the patient at about 175 mg/m².

Furthermore, the paclitaxel can be administered to the patient at various frequencies. In some embodiments, the paclitaxel is administered to the patient once per week (e.g., at about 60 mg/m² once per week or about 80 mg/m² once per week). In some embodiments, the paclitaxel is administered to the patient once every three weeks (e.g., at about 135 mg/m² once every three weeks or about 175 mg/m² once every three weeks). In some embodiments, the paclitaxel is administered to the patient by intravenous infusion once per week. In some embodiments, the paclitaxel is administered to the patient by intravenous infusion once per week for three consecutive weeks. In some embodiments, the method comprises administering paclitaxel to the patient by intravenous infusion for more than one cycle, wherein each cycle comprises administering paclitaxel to the patient by intravenous infusion once per week for three consecutive weeks (e.g., once per week for three consecutive weeks at about 60 mg/m² or about 80 mg/m²). In some embodiments, the method comprises not administering paclitaxel to the patient for at least one week between each cycle.

In some embodiments, the paclitaxel is administered to the patient by infusion once every three weeks. In some embodiments, the paclitaxel is administered to the patient for about 60 minutes once per week. In some embodiments, the paclitaxel is administered to the patient for about 3 hours once per week. In some embodiments, the paclitaxel is administered to the patient for about 60 minutes once every three weeks. In some embodiments, the paclitaxel is administered to the patient for about 3 hours once every three weeks. In some embodiments, the paclitaxel is administered to the patient by intravenous infusion for about 60 minutes once per week. In some embodiments, the paclitaxel is administered to the patient by infusion for about 3 hours once per week. In some embodiments, the paclitaxel is administered to the patient by intravenous infusion for about 60 minutes once every three weeks. In some embodiments, the paclitaxel is administered to the patient by infusion for about 3 hours once every three weeks. In some embodiments, the paclitaxel is continuously administered to the patient for about 60 minutes once per week. In some embodiments, the paclitaxel is continuously administered to the patient for about 3 hours once per week. In some embodiments, the paclitaxel is continuously administered to the patient for about 60 minutes once every three weeks. In some embodiments, the paclitaxel is continuously administered to the patient for about 3 hours once every three weeks. In some embodiments, the paclitaxel is continuously administered to the patient by infusion for about 60 minutes once per week. In some embodiments, the paclitaxel is continuously administered to the patient by intravenous infusion for about 3 hours once per week. In some embodiments, the paclitaxel is continuously administered to the patient by infusion for about 60 minutes once every three weeks. In some embodiments, the paclitaxel is continuously administered to the patient by intravenous infusion for about 3 hours once every three weeks.

In embodiments of the methods of achieving at least a stable disease condition in a patient having an endometrial adenocarcinoma described herein, the disclosure also contemplates administration of the compound of Formula (I) or a pharmaceutically acceptable salt to a patient in need thereof prior to (neo-adjuvant) or after (adjuvant) surgery (e.g., surgical treatment of a solid tumor). In some embodiments, the compound of Formula (I) or a pharmaceutically acceptable salt is administered as an adjuvant. In some embodiments, the compound of Formula (I) or a pharmaceutically acceptable salt is administered as a neo-adjuvant. In some embodiments, the compound of Formula (I) or a pharmaceutically acceptable salt is administered as a neo-adjuvant and an adjuvant. In some embodiments, the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 100 years. In some embodiments, administration of the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 5 years. In some embodiments, administration of the compound of Formula (I) or a pharmaceutically acceptable salt is administered to a patient in need thereof as a neo-adjuvant for a period of 3 months to 6 months, followed by administration of the compound of Formula (I) or a pharmaceutically acceptable salt as an adjuvant for a period from 1 day to 5 years. In some embodiments, no administration of the compound of Formula (I) or a pharmaceutically acceptable salt occurs prior to surgery. In some embodiments, the compound of Formula (I) or a pharmaceutically acceptable salt is administered as an adjuvant for a period from 1 day to 100 years. In some embodiments, the compound of Formula (I) or a pharmaceutically acceptable salt is administered as an adjuvant for a period from 1 day to 5 years.

### Mesothelioma

Also described herein are methods of treating mesothelioma in a patient in need thereof, comprising administering to the patient an effective amount of the compound of Formula (I)

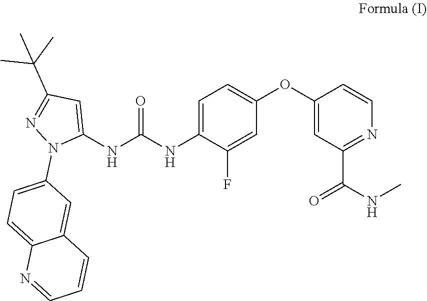 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In one embodiment, provided herein is a method of treating mesothelioma in a patient in need thereof, comprising administering to the patient about 50 mg to about 200 mg of the compound of Formula (I)

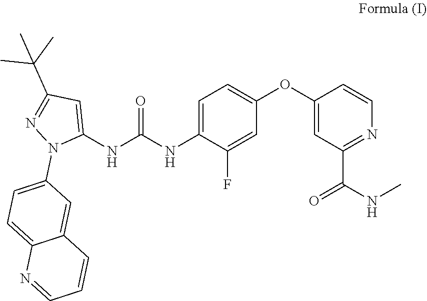 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof per day.

In embodiments of the methods for treating mesothelioma described herein, the compound of Formula (I) or pharmaceutically acceptable salt thereof can be orally administered to the patient in need thereof. In certain embodiments, the compound of Formula (I) or pharmaceutically acceptable salt thereof is administered to the patient daily. In some embodiments, the method comprises administering to the patient about 50 mg to about 150 mg of the compound of Formula (I) or pharmaceutically acceptable salt thereof per day. In some embodiments, the method comprises administering to the patient about 50 mg to about 100 mg of the compound of Formula (I) or pharmaceutically acceptable salt thereof per day. In some embodiments, the method comprises administering to the patient about 60 mg to about 200 mg of the compound of Formula (I) or pharmaceutically acceptable salt thereof per day. In some embodiments, the method comprises administering to the patient about 60 mg to about 150 mg of the compound of Formula (I) or pharmaceutically acceptable salt thereof per day. In some embodiments, the method comprises administering to the patient about 60 mg to about 100 mg of the compound of Formula (I) or pharmaceutically acceptable salt thereof per day. In some embodiments, the method comprises administering to the patient about 70 mg to about 200 mg of the compound of Formula (I) or pharmaceutically acceptable salt thereof per day. In some embodiments, the method comprises administering to the patient about 70 mg to about 150 mg of the compound of Formula (I) or pharmaceutically acceptable salt thereof per day. In some embodiments, the method comprises administering to the patient about 70 mg to about 100 mg of the compound of Formula (I) or pharmaceutically acceptable salt thereof per day. In some embodiments, the method comprises administering to the patient about 80 mg to about 200 mg of the compound of Formula (I) or pharmaceutically acceptable salt thereof per day. In some embodiments, the method comprises administering to the patient about 80 mg to about 150 mg of the compound of Formula (I) or pharmaceutically acceptable salt thereof per day. In some embodiments, the method comprises administering to the patient about 80 mg to about 100 mg of the compound of Formula (I) or pharmaceutically acceptable salt thereof per day. In some embodiments, the method comprises administering to the patient about 90 mg to about 200 mg of the compound of Formula (I) or pharmaceutically acceptable salt thereof per day. In some embodiments, the method comprises administering to the patient about 90 mg to about 150 mg of the compound of Formula (I) or pharmaceutically acceptable salt thereof per day. In some embodiments, the method comprises administering to the patient about 90 mg to about 100 mg of the compound of Formula (I) or pharmaceutically acceptable salt thereof per day. In some embodiments, the method comprises administering to the patient about 100 mg to about 200 mg of the compound of Formula (I) or pharmaceutically acceptable salt thereof per day. In some embodiments, the method comprises administering to the patient about 100 mg to about 150 mg of the compound of Formula (I) or pharmaceutically acceptable salt thereof per day.

In some embodiments, the method comprises administering to the patient about 50 mg of the compound of Formula (I) or pharmaceutically acceptable salt thereof per day. In some embodiments, the method comprises administering to the patient about 55 mg of the compound of Formula (I) or pharmaceutically acceptable salt thereof per day. In some embodiments, the method comprises administering to the patient about 60 mg of the compound of Formula (I) or pharmaceutically acceptable salt thereof per day. In some embodiments, the method comprises administering to the patient about 65 mg of the compound of Formula (I) or pharmaceutically acceptable salt thereof per day. In some embodiments, the method comprises administering to the patient about 70 mg of the compound of Formula (I) or pharmaceutically acceptable salt thereof per day. In some embodiments, the method comprises administering to the patient about 75 mg of the compound of Formula (I) or pharmaceutically acceptable salt thereof per day. In some embodiments, the method comprises administering to the patient about 80 mg of the compound of Formula (I) or pharmaceutically acceptable salt thereof per day. In some embodiments, the method comprises administering to the patient about 85 mg of the compound of Formula (I) or pharmaceutically acceptable salt thereof per day. In some embodiments, the method comprises administering to the patient about 90 mg of the compound of Formula (I) or pharmaceutically acceptable salt thereof per day. In some embodiments, the method comprises administering to the patient about 95 mg of the compound of Formula (I) or pharmaceutically acceptable salt thereof per day. In some embodiments, the method comprises administering to the patient about 100 mg of the compound of Formula (I) or pharmaceutically acceptable salt thereof per day. In some embodiments, the method comprises administering to the patient about 105 mg of the compound of Formula (I) or pharmaceutically acceptable salt thereof per day. In some embodiments, the method comprises administering to the patient about 110 mg of the compound of Formula (I) or pharmaceutically acceptable salt thereof per day. In some embodiments, the method comprises administering to the patient about 115 mg of the compound of Formula (I) or pharmaceutically acceptable salt thereof per day. In some embodiments, the method comprises administering to the patient about 120 mg of the compound of Formula (I) or pharmaceutically acceptable salt thereof per day. In some embodiments, the method comprises administering to the patient about 125 mg of the compound of Formula (I) or pharmaceutically acceptable salt thereof per day. In some embodiments, the method comprises administering to the patient about 130 mg of the compound of Formula (I) or pharmaceutically acceptable salt thereof per day. In some embodiments, the method comprises administering to the patient about 135 mg of the compound of Formula (I) or pharmaceutically acceptable salt thereof per day. In some embodiments, the method comprises administering to the patient about 140 mg of the compound of Formula (I) or pharmaceutically acceptable salt thereof per day. In some embodiments, the method comprises administering to the patient about 145 mg of the compound of Formula (I) or pharmaceutically acceptable salt thereof per day. In some embodiments, the method comprises administering to the patient about 150 mg of the compound of Formula (I) or pharmaceutically acceptable salt thereof per day. In some embodiments, the method comprises administering to the patient about 155 mg of the compound of Formula (I) or pharmaceutically acceptable salt thereof per day. In some embodiments, the method comprises administering to the patient about 160 mg of the compound of Formula (I) or pharmaceutically acceptable salt thereof per day. In some embodiments, the method comprises administering to the patient about 165 mg of the compound of Formula (I) or pharmaceutically acceptable salt thereof per day. In some embodiments, the method comprises administering to the patient about 170 mg of the compound of Formula (I) or pharmaceutically acceptable salt thereof per day. In some embodiments, the method comprises administering to the patient about 175 mg of the compound of Formula (I) or pharmaceutically acceptable salt thereof per day. In some embodiments, the method comprises administering to the patient about 180 mg of the compound of Formula (I) or pharmaceutically acceptable salt thereof per day. In some embodiments, the method comprises administering to the patient about 185 mg of the compound of Formula (I) or pharmaceutically acceptable salt thereof per day. In some embodiments, the method comprises administering to the patient about 190 mg of the compound of Formula (I) or pharmaceutically acceptable salt thereof per day. In some embodiments, the method comprises administering to the patient about 195 mg of the compound of Formula (I) or pharmaceutically acceptable salt thereof per day. In some embodiments, the method comprises administering to the patient about 200 mg of the compound of Formula (I) or pharmaceutically acceptable salt thereof per day.

In some embodiments, the method comprises administering to the patient about 50 mg to about 200 mg of the compound of Formula (I) or pharmaceutically acceptable salt thereof once per day. In some embodiments, the method comprises administering to the patient about 50 mg to about 150 mg of the compound of Formula (I) or pharmaceutically acceptable salt thereof once per day. In some embodiments, the method comprises administering to the patient about 50 mg to about 100 mg of the compound of Formula (I) or pharmaceutically acceptable salt thereof once per day. In some embodiments, the method comprises administering to the patient about 60 mg to about 200 mg of the compound of Formula (I) or pharmaceutically acceptable salt thereof once per day. In some embodiments, the method comprises administering to the patient about 60 mg to about 150 mg of the compound of Formula (I) or pharmaceutically acceptable salt thereof once per day. In some embodiments, the method comprises administering to the patient about 60 mg to about 100 mg of the compound of Formula (I) or pharmaceutically acceptable salt thereof once per day. In some embodiments, the method comprises administering to the patient about 70 mg to about 200 mg of the compound of Formula (I) or pharmaceutically acceptable salt thereof once per day. In some embodiments, the method comprises administering to the patient about 70 mg to about 150 mg of the compound of Formula (I) or pharmaceutically acceptable salt thereof once per day. In some embodiments, the method comprises administering to the patient about 70 mg to about 100 mg of the compound of Formula (I) or pharmaceutically acceptable salt thereof once per day. In some embodiments, the method comprises administering to the patient about 80 mg to about 200 mg of the compound of Formula (I) or pharmaceutically acceptable salt thereof once per day. In some embodiments, the method comprises administering to the patient about 80 mg to about 150 mg of the compound of Formula (I) or pharmaceutically acceptable salt thereof once per day. In some embodiments, the method comprises administering to the patient about 80 mg to about 100 mg of the compound of Formula (I) or pharmaceutically acceptable salt thereof once per day. In some embodiments, the method comprises administering to the patient about 90 mg to about 200 mg of the compound of Formula (I) or pharmaceutically acceptable salt thereof once per day. In some embodiments, the method comprises administering to the patient about 90 mg to about 150 mg of the compound of Formula (I) or pharmaceutically acceptable salt thereof once per day. In some embodiments, the method comprises administering to the patient about 90 mg to about 100 mg of the compound of Formula (I) or pharmaceutically acceptable salt thereof once per day. In some embodiments, the method comprises administering to the patient about 100 mg to about 200 mg of the compound of Formula (I) or pharmaceutically acceptable salt thereof once per day. In some embodiments, the method comprises administering to the patient about 100 mg to about 150 mg of the compound of Formula (I) or pharmaceutically acceptable salt thereof once per day.

In some embodiments, the method comprises administering to the patient about 50 mg of the compound of Formula (I) or pharmaceutically acceptable salt thereof once per day. In some embodiments, the method comprises administering to the patient about 55 mg of the compound of Formula (I) or pharmaceutically acceptable salt thereof once per day. In some embodiments, the method comprises administering to the patient about 60 mg of the compound of Formula (I) or pharmaceutically acceptable salt thereof once per day. In some embodiments, the method comprises administering to the patient about 65 mg of the compound of Formula (I) or pharmaceutically acceptable salt thereof once per day. In some embodiments, the method comprises administering to the patient about 70 mg of the compound of Formula (I) or pharmaceutically acceptable salt thereof once per day. In some embodiments, the method comprises administering to the patient about 75 mg of the compound of Formula (I) or pharmaceutically acceptable salt thereof once per day. In some embodiments, the method comprises administering to the patient about 80 mg of the compound of Formula (I) or pharmaceutically acceptable salt thereof once per day. In some embodiments, the method comprises administering to the patient about 85 mg of the compound of Formula (I) or pharmaceutically acceptable salt thereof once per day. In some embodiments, the method comprises administering to the patient about 90 mg of the compound of Formula (I) or pharmaceutically acceptable salt thereof once per day. In some embodiments, the method comprises administering to the patient about 95 mg of the compound of Formula (I) or pharmaceutically acceptable salt thereof once per day. In some embodiments, the method comprises administering to the patient about 100 mg of the compound of Formula (I) or pharmaceutically acceptable salt thereof once per day. In some embodiments, the method comprises administering to the patient about 105 mg of the compound of Formula (I) or pharmaceutically acceptable salt thereof once per day. In some embodiments, the method comprises administering to the patient about 110 mg of the compound of Formula (I) or pharmaceutically acceptable salt thereof once per day. In some embodiments, the method comprises administering to the patient about 115 mg of the compound of Formula (I) or pharmaceutically acceptable salt thereof once per day. In some embodiments, the method comprises administering to the patient about 120 mg of the compound of Formula (I) or pharmaceutically acceptable salt thereof once per day. In some embodiments, the method comprises administering to the patient about 125 mg of the compound of Formula (I) or pharmaceutically acceptable salt thereof once per day. In some embodiments, the method comprises administering to the patient about 130 mg of the compound of Formula (I) or pharmaceutically acceptable salt thereof once per day. In some embodiments, the method comprises administering to the patient about 135 mg of the compound of Formula (I) or pharmaceutically acceptable salt thereof once per day. In some embodiments, the method comprises administering to the patient about 140 mg of the compound of Formula (I) or pharmaceutically acceptable salt thereof once per day. In some embodiments, the method comprises administering to the patient about 145 mg of the compound of Formula (I) or pharmaceutically acceptable salt thereof once per day. In some embodiments, the method comprises administering to the patient about 150 mg of the compound of Formula (I) or pharmaceutically acceptable salt thereof once per day. In some embodiments, the method comprises administering to the patient about 155 mg of the compound of Formula (I) or pharmaceutically acceptable salt thereof once per day. In some embodiments, the method comprises administering to the patient about 160 mg of the compound of Formula (I) or pharmaceutically acceptable salt thereof once per day. In some embodiments, the method comprises administering to the patient about 165 mg of the compound of Formula (I) or pharmaceutically acceptable salt thereof once per day. In some embodiments, the method comprises administering to the patient about 170 mg of the compound of Formula (I) or pharmaceutically acceptable salt thereof once per day. In some embodiments, the method comprises administering to the patient about 175 mg of the compound of Formula (I) or pharmaceutically acceptable salt thereof once per day. In some embodiments, the method comprises administering to the patient about 180 mg of the compound of Formula (I) or pharmaceutically acceptable salt thereof once per day. In some embodiments, the method comprises administering to the patient about 185 mg of the compound of Formula (I) or pharmaceutically acceptable salt thereof once per day. In some embodiments, the method comprises administering to the patient about 190 mg of the compound of Formula (I) or pharmaceutically acceptable salt thereof once per day. In some embodiments, the method comprises administering to the patient about 195 mg of the compound of Formula (I) or pharmaceutically acceptable salt thereof once per day. In some embodiments, the method comprises administering to the patient about 200 mg of the compound of Formula (I) or pharmaceutically acceptable salt thereof once per day.

In some embodiments, the method comprises administering to the patient about 50 mg to about 100 mg of the compound of Formula (I) or pharmaceutically acceptable salt thereof twice per day. In some embodiments, the method comprises administering to the patient about 60 mg to about 100 mg of the compound of Formula (I) or pharmaceutically acceptable salt thereof twice per day. In some embodiments, the method comprises administering to the patient about 70 mg to about 100 mg of the compound of Formula (I) or pharmaceutically acceptable salt thereof twice per day. In some embodiments, the method comprises administering to the patient about 80 mg to about 100 mg of the compound of Formula (I) or pharmaceutically acceptable salt thereof twice per day. In some embodiments, the method comprises administering to the patient about 90 mg to about 100 mg of the compound of Formula (I) or pharmaceutically acceptable salt thereof twice per day.

In some embodiments, the method comprises administering to the patient about 50 mg of the compound of Formula (I) or pharmaceutically acceptable salt thereof twice per day. In some embodiments, the method comprises administering to the patient about 55 mg of the compound of Formula (I) or pharmaceutically acceptable salt thereof twice per day. In some embodiments, the method comprises administering to the patient about 60 mg of the compound of Formula (I) or pharmaceutically acceptable salt thereof twice per day. In some embodiments, the method comprises administering to the patient about 65 mg of the compound of Formula (I) or pharmaceutically acceptable salt thereof twice per day. In some embodiments, the method comprises administering to the patient about 70 mg of the compound of Formula (I) or pharmaceutically acceptable salt thereof twice per day. In some embodiments, the method comprises administering to the patient about 75 mg of the compound of Formula (I) or pharmaceutically acceptable salt thereof twice per day. In some embodiments, the method comprises administering to the patient about 80 mg of the compound of Formula (I) or pharmaceutically acceptable salt thereof twice per day. In some embodiments, the method comprises administering to the patient about 85 mg of the compound of Formula (I) or pharmaceutically acceptable salt thereof twice per day. In some embodiments, the method comprises administering to the patient about 90 mg of the compound of Formula (I) or pharmaceutically acceptable salt thereof twice per day. In some embodiments, the method comprises administering to the patient about 95 mg of the compound of Formula (I) or pharmaceutically acceptable salt thereof twice per day. In some embodiments, the method comprises administering to the patient about 100 mg of the compound of Formula (I) or pharmaceutically acceptable salt thereof twice per day.

The compound of Formula (I) or pharmaceutically acceptable salt thereof may, in another embodiment, be administered to the patient on an intermittent basis in the methods of treating mesothelioma described herein. In some embodiments, the compound of Formula (I) or pharmaceutically acceptable salt thereof is administered the patient every other day. In some embodiments, the compound of Formula (I) or pharmaceutically acceptable salt thereof is administered the patient every other day. In some embodiments, the compound of Formula (I) or pharmaceutically acceptable salt thereof is administered the patient once per week. In some embodiments, the compound of Formula (I) or pharmaceutically acceptable salt thereof is administered the patient twice per week. In some embodiments, the compound of Formula (I) or pharmaceutically acceptable salt thereof is administered the patient three times per week.

In some embodiments, the method comprises administering to the patient about 50 mg to about 200 mg of the compound of Formula (I) or pharmaceutically acceptable salt thereof once a week. In some embodiments, the method comprises administering to the patient about 50 mg to about 150 mg of the compound of Formula (I) or pharmaceutically acceptable salt thereof once a week. In some embodiments, the method comprises administering to the patient about 50 mg to about 100 mg of the compound of Formula (I) or pharmaceutically acceptable salt thereof once a week. In some embodiments, the method comprises administering to the patient about 60 mg to about 200 mg of the compound of Formula (I) or pharmaceutically acceptable salt thereof once a week. In some embodiments, the method comprises administering to the patient about 60 mg to about 150 mg of the compound of Formula (I) or pharmaceutically acceptable salt thereof once a week. In some embodiments, the method comprises administering to the patient about 60 mg to about 100 mg of the compound of Formula (I) or pharmaceutically acceptable salt thereof once a week. In some embodiments, the method comprises administering to the patient about 70 mg to about 200 mg of the compound of Formula (I) or pharmaceutically acceptable salt thereof once a week. In some embodiments, the method comprises administering to the patient about 70 mg to about 150 mg of the compound of Formula (I) or pharmaceutically acceptable salt thereof once a week. In some embodiments, the method comprises administering to the patient about 70 mg to about 100 mg of the compound of Formula (I) or pharmaceutically acceptable salt thereof once a week. In some embodiments, the method comprises administering to the patient about 80 mg to about 200 mg of the compound of Formula (I) or pharmaceutically acceptable salt thereof once a week. In some embodiments, the method comprises administering to the patient about 80 mg to about 150 mg of the compound of Formula (I) or pharmaceutically acceptable salt thereof once a week. In some embodiments, the method comprises administering to the patient about 80 mg to about 100 mg of the compound of Formula (I) or pharmaceutically acceptable salt thereof once a week. In some embodiments, the method comprises administering to the patient about 90 mg to about 200 mg of the compound of Formula (I) or pharmaceutically acceptable salt thereof once a week. In some embodiments, the method comprises administering to the patient about 90 mg to about 150 mg of the compound of Formula (I) or pharmaceutically acceptable salt thereof once a week. In some embodiments, the method comprises administering to the patient about 90 mg to about 100 mg of the compound of Formula (I) or pharmaceutically acceptable salt thereof once a week. In some embodiments, the method comprises administering to the patient about 100 mg to about 200 mg of the compound of Formula (I) or pharmaceutically acceptable salt thereof once a week. In some embodiments, the method comprises administering to the patient about 100 mg to about 150 mg of the compound of Formula (I) or pharmaceutically acceptable salt thereof once a week.

In some embodiments, the method comprises administering to the patient about 50 mg of the compound of Formula (I) or pharmaceutically acceptable salt thereof once a week. In some embodiments, the method comprises administering to the patient about 55 mg of the compound of Formula (I) or pharmaceutically acceptable salt thereof once a week. In some embodiments, the method comprises administering to the patient about 60 mg of the compound of Formula (I) or pharmaceutically acceptable salt thereof once a week. In some embodiments, the method comprises administering to the patient about 65 mg of the compound of Formula (I) or pharmaceutically acceptable salt thereof once a week. In some embodiments, the method comprises administering to the patient about 70 mg of the compound of Formula (I) or pharmaceutically acceptable salt thereof once a week. In some embodiments, the method comprises administering to the patient about 75 mg of the compound of Formula (I) or pharmaceutically acceptable salt thereof once a week. In some embodiments, the method comprises administering to the patient about 80 mg of the compound of Formula (I) or pharmaceutically acceptable salt thereof once a week. In some embodiments, the method comprises administering to the patient about 85 mg of the compound of Formula (I) or pharmaceutically acceptable salt thereof once a week. In some embodiments, the method comprises administering to the patient about 90 mg of the compound of Formula (I) or pharmaceutically acceptable salt thereof once a week. In some embodiments, the method comprises administering to the patient about 95 mg of the compound of Formula (I) or pharmaceutically acceptable salt thereof once a week. In some embodiments, the method comprises administering to the patient about 100 mg of the compound of Formula (I) or pharmaceutically acceptable salt thereof once a week. In some embodiments, the method comprises administering to the patient about 105 mg of the compound of Formula (I) or pharmaceutically acceptable salt thereof once a week. In some embodiments, the method comprises administering to the patient about 110 mg of the compound of Formula (I) or pharmaceutically acceptable salt thereof once a week. In some embodiments, the method comprises administering to the patient about 115 mg of the compound of Formula (I) or pharmaceutically acceptable salt thereof once a week. In some embodiments, the method comprises administering to the patient about 120 mg of the compound of Formula (I) or pharmaceutically acceptable salt thereof once a week. In some embodiments, the method comprises administering to the patient about 125 mg of the compound of Formula (I) or pharmaceutically acceptable salt thereof once a week. In some embodiments, the method comprises administering to the patient about 130 mg of the compound of Formula (I) or pharmaceutically acceptable salt thereof once a week. In some embodiments, the method comprises administering to the patient about 135 mg of the compound of Formula (I) or pharmaceutically acceptable salt thereof once a week. In some embodiments, the method comprises administering to the patient about 140 mg of the compound of Formula (I) or pharmaceutically acceptable salt thereof once a week. In some embodiments, the method comprises administering to the patient about 145 mg of the compound of Formula (I) or pharmaceutically acceptable salt thereof once a week. In some embodiments, the method comprises administering to the patient about 150 mg of the compound of Formula (I) or pharmaceutically acceptable salt thereof once a week. In some embodiments, the method comprises administering to the patient about 155 mg of the compound of Formula (I) or pharmaceutically acceptable salt thereof once a week. In some embodiments, the method comprises administering to the patient about 160 mg of the compound of Formula (I) or pharmaceutically acceptable salt thereof once a week. In some embodiments, the method comprises administering to the patient about 165 mg of the compound of Formula (I) or pharmaceutically acceptable salt thereof once a week. In some embodiments, the method comprises administering to the patient about 170 mg of the compound of Formula (I) or pharmaceutically acceptable salt thereof once a week. In some embodiments, the method comprises administering to the patient about 175 mg of the compound of Formula (I) or pharmaceutically acceptable salt thereof once a week. In some embodiments, the method comprises administering to the patient about 180 mg of the compound of Formula (I) or pharmaceutically acceptable salt thereof once a week. In some embodiments, the method comprises administering to the patient about 185 mg of the compound of Formula (I) or pharmaceutically acceptable salt thereof once a week. In some embodiments, the method comprises administering to the patient about 190 mg of the compound of Formula (I) or pharmaceutically acceptable salt thereof once a week. In some embodiments, the method comprises administering to the patient about 195 mg of the compound of Formula (I) or pharmaceutically acceptable salt thereof once a week. In some embodiments, the method comprises administering to the patient about 200 mg of the compound of Formula (I) or pharmaceutically acceptable salt thereof once a week.

In some embodiments, the method comprises administering to the patient about 50 mg to about 200 mg of the compound of Formula (I) or pharmaceutically acceptable salt thereof twice a week. In some embodiments, the method comprises administering to the patient about 50 mg to about 150 mg of the compound of Formula (I) or pharmaceutically acceptable salt thereof twice a week. In some embodiments, the method comprises administering to the patient about 50 mg to about 100 mg of the compound of Formula (I) or pharmaceutically acceptable salt thereof twice a week. In some embodiments, the method comprises administering to the patient about 60 mg to about 200 mg of the compound of Formula (I) or pharmaceutically acceptable salt thereof twice a week. In some embodiments, the method comprises administering to the patient about 60 mg to about 150 mg of the compound of Formula (I) or pharmaceutically acceptable salt thereof twice a week. In some embodiments, the method comprises administering to the patient about 60 mg to about 100 mg of the compound of Formula (I) or pharmaceutically acceptable salt thereof twice a week. In some embodiments, the method comprises administering to the patient about 70 mg to about 200 mg of the compound of Formula (I) or pharmaceutically acceptable salt thereof twice a week. In some embodiments, the method comprises administering to the patient about 70 mg to about 150 mg of the compound of Formula (I) or pharmaceutically acceptable salt thereof twice a week. In some embodiments, the method comprises administering to the patient about 70 mg to about 100 mg of the compound of Formula (I) or pharmaceutically acceptable salt thereof twice a week. In some embodiments, the method comprises administering to the patient about 80 mg to about 200 mg of the compound of Formula (I) or pharmaceutically acceptable salt thereof twice a week. In some embodiments, the method comprises administering to the patient about 80 mg to about 150 mg of the compound of Formula (I) or pharmaceutically acceptable salt thereof twice a week. In some embodiments, the method comprises administering to the patient about 80 mg to about 100 mg of the compound of Formula (I) or pharmaceutically acceptable salt thereof twice a week. In some embodiments, the method comprises administering to the patient about 90 mg to about 200 mg of the compound of Formula (I) or pharmaceutically acceptable salt thereof twice a week. In some embodiments, the method comprises administering to the patient about 90 mg to about 150 mg of the compound of Formula (I) or pharmaceutically acceptable salt thereof twice a week. In some embodiments, the method comprises administering to the patient about 90 mg to about 100 mg of the compound of Formula (I) or pharmaceutically acceptable salt thereof twice a week. In some embodiments, the method comprises administering to the patient about 100 mg to about 200 mg of the compound of Formula (I) or pharmaceutically acceptable salt thereof twice a week. In some embodiments, the method comprises administering to the patient about 100 mg to about 150 mg of the compound of Formula (I) or pharmaceutically acceptable salt thereof twice a week.

In some embodiments, the method comprises administering to the patient about 50 mg of the compound of Formula (I) or pharmaceutically acceptable salt thereof twice a week. In some embodiments, the method comprises administering to the patient about 55 mg of the compound of Formula (I) or pharmaceutically acceptable salt thereof twice a week. In some embodiments, the method comprises administering to the patient about 60 mg of the compound of Formula (I) or pharmaceutically acceptable salt thereof twice a week. In some embodiments, the method comprises administering to the patient about 65 mg of the compound of Formula (I) or pharmaceutically acceptable salt thereof twice a week. In some embodiments, the method comprises administering to the patient about 70 mg of the compound of Formula (I) or pharmaceutically acceptable salt thereof twice a week. In some embodiments, the method comprises administering to the patient about 75 mg of the compound of Formula (I) or pharmaceutically acceptable salt thereof twice a week. In some embodiments, the method comprises administering to the patient about 80 mg of the compound of Formula (I) or pharmaceutically acceptable salt thereof twice a week. In some embodiments, the method comprises administering to the patient about 85 mg of the compound of Formula (I) or pharmaceutically acceptable salt thereof twice a week. In some embodiments, the method comprises administering to the patient about 90 mg of the compound of Formula (I) or pharmaceutically acceptable salt thereof twice a week. In some embodiments, the method comprises administering to the patient about 95 mg of the compound of Formula (I) or pharmaceutically acceptable salt thereof twice a week. In some embodiments, the method comprises administering to the patient about 100 mg of the compound of Formula (I) or pharmaceutically acceptable salt thereof twice a week. In some embodiments, the method comprises administering to the patient about 105 mg of the compound of Formula (I) or pharmaceutically acceptable salt thereof twice a week. In some embodiments, the method comprises administering to the patient about 110 mg of the compound of Formula (I) or pharmaceutically acceptable salt thereof twice a week. In some embodiments, the method comprises administering to the patient about 115 mg of the compound of Formula (I) or pharmaceutically acceptable salt thereof twice a week. In some embodiments, the method comprises administering to the patient about 120 mg of the compound of Formula (I) or pharmaceutically acceptable salt thereof twice a week. In some embodiments, the method comprises administering to the patient about 125 mg of the compound of Formula (I) or pharmaceutically acceptable salt thereof twice a week. In some embodiments, the method comprises administering to the patient about 130 mg of the compound of Formula (I) or pharmaceutically acceptable salt thereof twice a week. In some embodiments, the method comprises administering to the patient about 135 mg of the compound of Formula (I) or pharmaceutically acceptable salt thereof twice a week. In some embodiments, the method comprises administering to the patient about 140 mg of the compound of Formula (I) or pharmaceutically acceptable salt thereof twice a week. In some embodiments, the method comprises administering to the patient about 145 mg of the compound of Formula (I) or pharmaceutically acceptable salt thereof twice a week. In some embodiments, the method comprises administering to the patient about 150 mg of the compound of Formula (I) or pharmaceutically acceptable salt thereof twice a week. In some embodiments, the method comprises administering to the patient about 155 mg of the compound of Formula (I) or pharmaceutically acceptable salt thereof twice a week. In some embodiments, the method comprises administering to the patient about 160 mg of the compound of Formula (I) or pharmaceutically acceptable salt thereof twice a week. In some embodiments, the method comprises administering to the patient about 165 mg of the compound of Formula (I) or pharmaceutically acceptable salt thereof twice a week. In some embodiments, the method comprises administering to the patient about 170 mg of the compound of Formula (I) or pharmaceutically acceptable salt thereof twice a week. In some embodiments, the method comprises administering to the patient about 175 mg of the compound of Formula (I) or pharmaceutically acceptable salt thereof twice a week. In some embodiments, the method comprises administering to the patient about 180 mg of the compound of Formula (I) or pharmaceutically acceptable salt thereof twice a week. In some embodiments, the method comprises administering to the patient about 185 mg of the compound of Formula (I) or pharmaceutically acceptable salt thereof twice a week. In some embodiments, the method comprises administering to the patient about 190 mg of the compound of Formula (I) or pharmaceutically acceptable salt thereof twice a week. In some embodiments, the method comprises administering to the patient about 195 mg of the compound of Formula (I) or pharmaceutically acceptable salt thereof twice a week. In some embodiments, the method comprises administering to the patient about 200 mg of the compound of Formula (I) or pharmaceutically acceptable salt thereof twice a week.

In some embodiments, the method comprises administering to the patient about 50 mg to about 200 mg of the compound of Formula (I) or pharmaceutically acceptable salt thereof three times a week. In some embodiments, the method comprises administering to the patient about 50 mg to about 150 mg of the compound of Formula (I) or pharmaceutically acceptable salt thereof three times a week. In some embodiments, the method comprises administering to the patient about 50 mg to about 100 mg of the compound of Formula (I) or pharmaceutically acceptable salt thereof three times a week. In some embodiments, the method comprises administering to the patient about 60 mg to about 200 mg of the compound of Formula (I) or pharmaceutically acceptable salt thereof three times a week. In some embodiments, the method comprises administering to the patient about 60 mg to about 150 mg of the compound of Formula (I) or pharmaceutically acceptable salt thereof three times a week. In some embodiments, the method comprises administering to the patient about 60 mg to about 100 mg of the compound of Formula (I) or pharmaceutically acceptable salt thereof three times a week. In some embodiments, the method comprises administering to the patient about 70 mg to about 200 mg of the compound of Formula (I) or pharmaceutically acceptable salt thereof three times a week. In some embodiments, the method comprises administering to the patient about 70 mg to about 150 mg of the compound of Formula (I) or pharmaceutically acceptable salt thereof three times a week. In some embodiments, the method comprises administering to the patient about 70 mg to about 100 mg of the compound of Formula (I) or pharmaceutically acceptable salt thereof three times a week. In some embodiments, the method comprises administering to the patient about 80 mg to about 200 mg of the compound of Formula (I) or pharmaceutically acceptable salt thereof three times a week. In some embodiments, the method comprises administering to the patient about 80 mg to about 150 mg of the compound of Formula (I) or pharmaceutically acceptable salt thereof three times a week. In some embodiments, the method comprises administering to the patient about 80 mg to about 100 mg of the compound of Formula (I) or pharmaceutically acceptable salt thereof three times a week. In some embodiments, the method comprises administering to the patient about 90 mg to about 200 mg of the compound of Formula (I) or pharmaceutically acceptable salt thereof three times a week. In some embodiments, the method comprises administering to the patient about 90 mg to about 150 mg of the compound of Formula (I) or pharmaceutically acceptable salt thereof three times a week. In some embodiments, the method comprises administering to the patient about 90 mg to about 100 mg of the compound of Formula (I) or pharmaceutically acceptable salt thereof three times a week. In some embodiments, the method comprises administering to the patient about 100 mg to about 200 mg of the compound of Formula (I) or pharmaceutically acceptable salt thereof three times a week. In some embodiments, the method comprises administering to the patient about 100 mg to about 150 mg of the compound of Formula (I) or pharmaceutically acceptable salt thereof three times a week.

In some embodiments, the method comprises administering to the patient about 50 mg of the compound of Formula (I) or pharmaceutically acceptable salt thereof three times a week. In some embodiments, the method comprises administering to the patient about 55 mg of the compound of Formula (I) or pharmaceutically acceptable salt thereof three times a week. In some embodiments, the method comprises administering to the patient about 60 mg of the compound of Formula (I) or pharmaceutically acceptable salt thereof three times a week. In some embodiments, the method comprises administering to the patient about 65 mg of the compound of Formula (I) or pharmaceutically acceptable salt thereof three times a week. In some embodiments, the method comprises administering to the patient about 70 mg of the compound of Formula (I) or pharmaceutically acceptable salt thereof three times a week. In some embodiments, the method comprises administering to the patient about 75 mg of the compound of Formula (I) or pharmaceutically acceptable salt thereof three times a week. In some embodiments, the method comprises administering to the patient about 80 mg of the compound of Formula (I) or pharmaceutically acceptable salt thereof three times a week. In some embodiments, the method comprises administering to the patient about 85 mg of the compound of Formula (I) or pharmaceutically acceptable salt thereof three times a week. In some embodiments, the method comprises administering to the patient about 90 mg of the compound of Formula (I) or pharmaceutically acceptable salt thereof three times a week. In some embodiments, the method comprises administering to the patient about 95 mg of the compound of Formula (I) or pharmaceutically acceptable salt thereof three times a week. In some embodiments, the method comprises administering to the patient about 100 mg of the compound of Formula (I) or pharmaceutically acceptable salt thereof three times a week. In some embodiments, the method comprises administering to the patient about 105 mg of the compound of Formula (I) or pharmaceutically acceptable salt thereof three times a week. In some embodiments, the method comprises administering to the patient about 110 mg of the compound of Formula (I) or pharmaceutically acceptable salt thereof three times a week. In some embodiments, the method comprises administering to the patient about 115 mg of the compound of Formula (I) or pharmaceutically acceptable salt thereof three times a week. In some embodiments, the method comprises administering to the patient about 120 mg of the compound of Formula (I) or pharmaceutically acceptable salt thereof three times a week. In some embodiments, the method comprises administering to the patient about 125 mg of the compound of Formula (I) or pharmaceutically acceptable salt thereof three times a week. In some embodiments, the method comprises administering to the patient about 130 mg of the compound of Formula (I) or pharmaceutically acceptable salt thereof three times a week. In some embodiments, the method comprises administering to the patient about 135 mg of the compound of Formula (I) or pharmaceutically acceptable salt thereof three times a week. In some embodiments, the method comprises administering to the patient about 140 mg of the compound of Formula (I) or pharmaceutically acceptable salt thereof three times a week. In some embodiments, the method comprises administering to the patient about 145 mg of the compound of Formula (I) or pharmaceutically acceptable salt thereof three times a week. In some embodiments, the method comprises administering to the patient about 150 mg of the compound of Formula (I) or pharmaceutically acceptable salt thereof three times a week. In some embodiments, the method comprises administering to the patient about 155 mg of the compound of Formula (I) or pharmaceutically acceptable salt thereof three times a week. In some embodiments, the method comprises administering to the patient about 160 mg of the compound of Formula (I) or pharmaceutically acceptable salt thereof three times a week. In some embodiments, the method comprises administering to the patient about 165 mg of the compound of Formula (I) or pharmaceutically acceptable salt thereof three times a week. In some embodiments, the method comprises administering to the patient about 170 mg of the compound of Formula (I) or pharmaceutically acceptable salt thereof three times a week. In some embodiments, the method comprises administering to the patient about 175 mg of the compound of Formula (I) or pharmaceutically acceptable salt thereof three times a week. In some embodiments, the method comprises administering to the patient about 180 mg of the compound of Formula (I) or pharmaceutically acceptable salt thereof three times a week. In some embodiments, the method comprises administering to the patient about 185 mg of the compound of Formula (I) or pharmaceutically acceptable salt thereof three times a week. In some embodiments, the method comprises administering to the patient about 190 mg of the compound of Formula (I) or pharmaceutically acceptable salt thereof three times a week. In some embodiments, the method comprises administering to the patient about 195 mg of the compound of Formula (I) or pharmaceutically acceptable salt thereof three times a week. In some embodiments, the method comprises administering to the patient about 200 mg of the compound of Formula (I) or pharmaceutically acceptable salt thereof three times a week.

In some embodiments, the method comprises administering to the patient about 50 mg to about 200 mg of the compound of Formula (I) or pharmaceutically acceptable salt thereof every other day. In some embodiments, the method comprises administering to the patient about 50 mg to about 150 mg of the compound of Formula (I) or pharmaceutically acceptable salt thereof every other day. In some embodiments, the method comprises administering to the patient about 50 mg to about 100 mg of the compound of Formula (I) or pharmaceutically acceptable salt thereof every other day. In some embodiments, the method comprises administering to the patient about 60 mg to about 200 mg of the compound of Formula (I) or pharmaceutically acceptable salt thereof every other day. In some embodiments, the method comprises administering to the patient about 60 mg to about 150 mg of the compound of Formula (I) or pharmaceutically acceptable salt thereof every other day. In some embodiments, the method comprises administering to the patient about 60 mg to about 100 mg of the compound of Formula (I) or pharmaceutically acceptable salt thereof every other day. In some embodiments, the method comprises administering to the patient about 70 mg to about 200 mg of the compound of Formula (I) or pharmaceutically acceptable salt thereof every other day. In some embodiments, the method comprises administering to the patient about 70 mg to about 150 mg of the compound of Formula (I) or pharmaceutically acceptable salt thereof every other day. In some embodiments, the method comprises administering to the patient about 70 mg to about 100 mg of the compound of Formula (I) or pharmaceutically acceptable salt thereof every other day. In some embodiments, the method comprises administering to the patient about 80 mg to about 200 mg of the compound of Formula (I) or pharmaceutically acceptable salt thereof every other day. In some embodiments, the method comprises administering to the patient about 80 mg to about 150 mg of the compound of Formula (I) or pharmaceutically acceptable salt thereof every other day. In some embodiments, the method comprises administering to the patient about 80 mg to about 100 mg of the compound of Formula (I) or pharmaceutically acceptable salt thereof every other day. In some embodiments, the method comprises administering to the patient about 90 mg to about 200 mg of the compound of Formula (I) or pharmaceutically acceptable salt thereof every other day. In some embodiments, the method comprises administering to the patient about 90 mg to about 150 mg of the compound of Formula (I) or pharmaceutically acceptable salt thereof every other day. In some embodiments, the method comprises administering to the patient about 90 mg to about 100 mg of the compound of Formula (I) or pharmaceutically acceptable salt thereof every other day. In some embodiments, the method comprises administering to the patient about 100 mg to about 200 mg of the compound of Formula (I) or pharmaceutically acceptable salt thereof every other day. In some embodiments, the method comprises administering to the patient about 100 mg to about 150 mg of the compound of Formula (I) or pharmaceutically acceptable salt thereof every other day.

In some embodiments, the method comprises administering to the patient about 50 mg of the compound of Formula (I) or pharmaceutically acceptable salt thereof every other day. In some embodiments, the method comprises administering to the patient about 55 mg of the compound of Formula (I) or pharmaceutically acceptable salt thereof every other day. In some embodiments, the method comprises administering to the patient about 60 mg of the compound of Formula (I) or pharmaceutically acceptable salt thereof every other day. In some embodiments, the method comprises administering to the patient about 65 mg of the compound of Formula (I) or pharmaceutically acceptable salt thereof every other day. In some embodiments, the method comprises administering to the patient about 70 mg of the compound of Formula (I) or pharmaceutically acceptable salt thereof every other day. In some embodiments, the method comprises administering to the patient about 75 mg of the compound of Formula (I) or pharmaceutically acceptable salt thereof every other day. In some embodiments, the method comprises administering to the patient about 80 mg of the compound of Formula (I) or pharmaceutically acceptable salt thereof every other day. In some embodiments, the method comprises administering to the patient about 85 mg of the compound of Formula (I) or pharmaceutically acceptable salt thereof every other day. In some embodiments, the method comprises administering to the patient about 90 mg of the compound of Formula (I) or pharmaceutically acceptable salt thereof every other day. In some embodiments, the method comprises administering to the patient about 95 mg of the compound of Formula (I) or pharmaceutically acceptable salt thereof every other day. In some embodiments, the method comprises administering to the patient about 100 mg of the compound of Formula (I) or pharmaceutically acceptable salt thereof every other day. In some embodiments, the method comprises administering to the patient about 105 mg of the compound of Formula (I) or pharmaceutically acceptable salt thereof every other day. In some embodiments, the method comprises administering to the patient about 110 mg of the compound of Formula (I) or pharmaceutically acceptable salt thereof every other day. In some embodiments, the method comprises administering to the patient about 115 mg of the compound of Formula (I) or pharmaceutically acceptable salt thereof every other day. In some embodiments, the method comprises administering to the patient about 120 mg of the compound embodiments, the method comprises administering to the patient about 125 mg of the compound of Formula (I) or pharmaceutically acceptable salt thereof every other day. In some embodiments, the method comprises administering to the patient about 130 mg of the compound of Formula (I) or pharmaceutically acceptable salt thereof every other day. In some embodiments, the method comprises administering to the patient about 135 mg of the compound of Formula (I) or pharmaceutically acceptable salt thereof every other day. In some embodiments, the method comprises administering to the patient about 140 mg of the compound of Formula (I) or pharmaceutically acceptable salt thereof every other day. In some embodiments, the method comprises administering to the patient about 145 mg of the compound of Formula (I) or pharmaceutically acceptable salt thereof every other day. In some embodiments, the method comprises administering to the patient about 150 mg of the compound of Formula (I) or pharmaceutically acceptable salt thereof every other day. In some embodiments, the method comprises administering to the patient about 155 mg of the compound of Formula (I) or pharmaceutically acceptable salt thereof every other day. In some embodiments, the method comprises administering to the patient about 160 mg of the compound of Formula (I) or pharmaceutically acceptable salt thereof every other day. In some embodiments, the method comprises administering to the patient about 165 mg of the compound of Formula (I) or pharmaceutically acceptable salt thereof every other day. In some embodiments, the method comprises administering to the patient about 170 mg of the compound of Formula (I) or pharmaceutically acceptable salt thereof every other day. In some embodiments, the method comprises administering to the patient about 175 mg of the compound of Formula (I) or pharmaceutically acceptable salt thereof every other day. In some embodiments, the method comprises administering to the patient about 180 mg of the compound of Formula (I) or pharmaceutically acceptable salt thereof every other day. In some embodiments, the method comprises administering to the patient about 185 mg of the compound of Formula (I) or pharmaceutically acceptable salt thereof every other day. In some embodiments, the method comprises administering to the patient about 190 mg of the compound of Formula (I) or pharmaceutically acceptable salt thereof every other day. In some embodiments, the method comprises administering to the patient about 195 mg of the compound of Formula (I) or pharmaceutically acceptable salt thereof every other day. In some embodiments, the method comprises administering to the patient about 200 mg of the compound of Formula (I) or pharmaceutically acceptable salt thereof every other day.

In some embodiments, the method further comprises administering a combination agent to the patient in need thereof. In some embodiments, the combination agent is carboplatin. In some embodiments, the combination agent is paclitaxel. In some embodiments, the method further comprises administering pemetrexed to the patient in need thereof. In some embodiments, the combination agent is intravenously administered to the patient in need thereof. In some embodiments, the method further comprises administering one mor more combination agents to the patient in need thereof. In some embodiments, the method further comprises administering carboplatin and paclitaxel.

In some embodiments, the patient has previously been administered at least one prior line of therapy. In some embodiments, the at least one prior line of therapy is a platinum-based therapy. In some embodiments, the patient has previously been administered carboplatin and/or pemetrexed. In some embodiments, the patient has previously been administered cisplatin and/or pemetrexed.

The one or more combination agents administered to a patient in the methods of treating mesothelioma described herein can be dosed to achieve various concentrations of the combination agents in the patient's plasma, which can be quantified by the area under the curve (AUC) for each combination agent administered, e.g., intravenously administered. In some embodiments, the combination agent (e.g., carboplatin) is intravenously administered to the patient in an amount sufficient to provide an AUC of about 5 to about 7 (e.g., an AUC of about 5 or about 6).

In some embodiments, the paclitaxel is intravenously administered to the patient, e.g., by intravenous infusion. The intravenously administered paclitaxel can be administered to a patient at various dose levels. In some embodiments, the paclitaxel is administered to the patient at about 50 mg/m² to about 150 mg/m². In some embodiments, the paclitaxel is administered to the patient at about 50 mg/m² to about 100 mg/m². In some embodiments, the paclitaxel is administered to the patient at about 60 mg/m² to about 80 mg/m². In some embodiments, the paclitaxel is administered to the patient at about 60 mg/m². In some embodiments, the paclitaxel is administered to the patient at about 80 mg/m². In some embodiments, the paclitaxel is administered to the patient at about 135 mg/m². In some embodiments, the paclitaxel is administered to the patient at about 175 mg/m².

Furthermore, the paclitaxel can be administered to the patient at various frequencies. In some embodiments, the paclitaxel is administered to the patient once per week (e.g., at about 60 mg/m² once per week or about 80 mg/m² once per week). In some embodiments, the paclitaxel is administered to the patient once every three weeks (e.g., at about 135 mg/m² once every three weeks or about 175 mg/m² once every three weeks). In some embodiments, the paclitaxel is administered to the patient by intravenous infusion once per week. In some embodiments, the paclitaxel is administered to the patient by intravenous infusion once per week for three consecutive weeks. In some embodiments, the method comprises administering paclitaxel to the patient by intravenous infusion for more than one cycle, wherein each cycle comprises administering paclitaxel to the patient by intravenous infusion once per week for three consecutive weeks (e.g., once per week for three consecutive weeks at about 60 mg/m² or about 80 mg/m²). In some embodiments, the method comprises not administering paclitaxel to the patient for at least one week between each cycle.

In some embodiments, the paclitaxel is administered to the patient by infusion once every three weeks. In some embodiments, the paclitaxel is administered to the patient for about 60 minutes once per week. In some embodiments, the paclitaxel is administered to the patient for about 3 hours once per week. In some embodiments, the paclitaxel is administered to the patient for about 60 minutes once every three weeks. In some embodiments, the paclitaxel is administered to the patient for about 3 hours once every three weeks. In some embodiments, the paclitaxel is administered to the patient by intravenous infusion for about 60 minutes once per week. In some embodiments, the paclitaxel is administered to the patient by infusion for about 3 hours once per week. In some embodiments, the paclitaxel is administered to the patient by intravenous infusion for about 60 minutes once every three weeks. In some embodiments, the paclitaxel is administered to the patient by infusion for about 3 hours once every three weeks. In some embodiments, the paclitaxel is continuously administered to the patient for about 60 minutes once per week. In some embodiments, the paclitaxel is continuously administered to the patient for about 3 hours once per week. In some embodiments, the paclitaxel is continuously administered to the patient for about 60 minutes once every three weeks. In some embodiments, the paclitaxel is continuously administered to the patient for about 3 hours once every three weeks. In some embodiments, the paclitaxel is continuously administered to the patient by infusion for about 60 minutes once per week. In some embodiments, the paclitaxel is continuously administered to the patient by intravenous infusion for about 3 hours once per week. In some embodiments, the paclitaxel is continuously administered to the patient by infusion for about 60 minutes once every three weeks. In some embodiments, the paclitaxel is continuously administered to the patient by intravenous infusion for about 3 hours once every three weeks.

A method of treating mesothelioma described herein can further comprise reducing each of the amount of the compound of Formula (I) or pharmaceutically acceptable salt thereof and the combination agent administered to the patient after one or more months of administering said compound of Formula (I) or pharmaceutically acceptable salt thereof and said combination agent to the patient in need thereof.

In embodiments of the methods of treating mesothelioma described herein, the disclosure also contemplates administration of the compound of Formula (I) or a pharmaceutically acceptable salt to a patient in need thereof prior to (neo-adjuvant) or after (adjuvant) surgery (e.g., surgical treatment of a solid tumor). In some embodiments, the compound of Formula (I) or a pharmaceutically acceptable salt is administered as an adjuvant. In some embodiments, the compound of Formula (I) or a pharmaceutically acceptable salt is administered as a neo-adjuvant. In some embodiments, the compound of Formula (I) or a pharmaceutically acceptable salt is administered as a neo-adjuvant and an adjuvant. In some embodiments, the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 100 years. In some embodiments, administration of the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 5 years. In some embodiments, administration of the compound of Formula (I) or a pharmaceutically acceptable salt is administered to a patient in need thereof as a neo-adjuvant for a period of 3 months to 6 months, followed by administration of the compound of Formula (I) or a pharmaceutically acceptable salt as an adjuvant for a period from 1 day to 5 years. In some embodiments, no administration of the compound of Formula (I) or a pharmaceutically acceptable salt occurs prior to surgery. In some embodiments, the compound of Formula (I) or a pharmaceutically acceptable salt is administered as an adjuvant for a period from 1 day to 100 years. In some embodiments, the compound of Formula (I) or a pharmaceutically acceptable salt is administered as an adjuvant for a period from 1 day to 5 years.

In another embodiment, provided herein is a method for achieving at least a partial response in a patient having mesothelioma, comprising administering to the patient an effective amount of the compound of Formula (I)

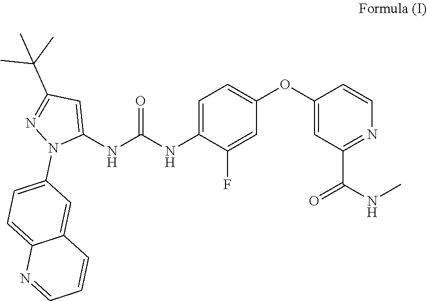 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In some embodiments, the methods further comprise administering a combination agent to the patient in need thereof. In some embodiments, the combination agent is carboplatin. In some embodiments, the combination agent is paclitaxel. In some embodiments, the method further comprises administering pemetrexed to the patient in need thereof. In some embodiments, the method further comprises administering one or more combination agents to the patient in need thereof. In some embodiments, the method further comprises administering carboplatin and paclitaxel.

In some embodiments, the patient has previously been administered at least one prior line of therapy. In some embodiments, the therapy is a platinum-based therapy. In some embodiments, the patient previously been administered carboplatin and/or pemetrexed. In some embodiments, the patient previously been administered cisplatin and/or pemetrexed.

In embodiments of the methods for treating mesothelioma described herein, the compound of Formula (I) or pharmaceutically acceptable salt thereof can be orally administered to the patient in need thereof. In certain embodiments, the compound of Formula (I) or pharmaceutically acceptable salt thereof is administered to the patient daily. In some embodiments, the method comprises administering to the patient about 50 mg to about 150 mg of the compound of Formula (I) or pharmaceutically acceptable salt thereof per day. In some embodiments, the method comprises administering to the patient about 50 mg to about 100 mg of the compound of Formula (I) or pharmaceutically acceptable salt thereof per day. In some embodiments, the method comprises administering to the patient about 60 mg to about 200 mg of the compound of Formula (I) or pharmaceutically acceptable salt thereof per day. In some embodiments, the method comprises administering to the patient about 60 mg to about 150 mg of the compound of Formula (I) or pharmaceutically acceptable salt thereof per day. In some embodiments, the method comprises administering to the patient about 60 mg to about 100 mg of the compound of Formula (I) or pharmaceutically acceptable salt thereof per day. In some embodiments, the method comprises administering to the patient about 70 mg to about 200 mg of the compound of Formula (I) or pharmaceutically acceptable salt thereof per day. In some embodiments, the method comprises administering to the patient about 70 mg to about 150 mg of the compound of Formula (I) or pharmaceutically acceptable salt thereof per day. In some embodiments, the method comprises administering to the patient about 70 mg to about 100 mg of the compound of Formula (I) or pharmaceutically acceptable salt thereof per day. In some embodiments, the method comprises administering to the patient about 80 mg to about 200 mg of the compound of Formula (I) or pharmaceutically acceptable salt thereof per day. In some embodiments, the method comprises administering to the patient about 80 mg to about 150 mg of the compound of Formula (I) or pharmaceutically acceptable salt thereof per day. In some embodiments, the method comprises administering to the patient about 80 mg to about 100 mg of the compound of Formula (I) or pharmaceutically acceptable salt thereof per day. In some embodiments, the method comprises administering to the patient about 90 mg to about 200 mg of the compound of Formula (I) or pharmaceutically acceptable salt thereof per day. In some embodiments, the method comprises administering to the patient about 90 mg to about 150 mg of the compound of Formula (I) or pharmaceutically acceptable salt thereof per day. In some embodiments, the method comprises administering to the patient about 90 mg to about 100 mg of the compound of Formula (I) or pharmaceutically acceptable salt thereof per day. In some embodiments, the method comprises administering to the patient about 100 mg to about 200 mg of the compound of Formula (I) or pharmaceutically acceptable salt thereof per day. In some embodiments, the method comprises administering to the patient about 100 mg to about 150 mg of the compound of Formula (I) or pharmaceutically acceptable salt thereof per day.

In some embodiments, the method comprises administering to the patient about 50 mg of the compound of Formula (I) or pharmaceutically acceptable salt thereof per day. In some embodiments, the method comprises administering to the patient about 55 mg of the compound of Formula (I) or pharmaceutically acceptable salt thereof per day. In some embodiments, the method comprises administering to the patient about 60 mg of the compound of Formula (I) or pharmaceutically acceptable salt thereof per day. In some embodiments, the method comprises administering to the patient about 65 mg of the compound of Formula (I) or pharmaceutically acceptable salt thereof per day. In some embodiments, the method comprises administering to the patient about 70 mg of the compound of Formula (I) or pharmaceutically acceptable salt thereof per day. In some embodiments, the method comprises administering to the patient about 75 mg of the compound of Formula (I) or pharmaceutically acceptable salt thereof per day. In some embodiments, the method comprises administering to the patient about 80 mg of the compound of Formula (I) or pharmaceutically acceptable salt thereof per day. In some embodiments, the method comprises administering to the patient about 85 mg of the compound of Formula (I) or pharmaceutically acceptable salt thereof per day. In some embodiments, the method comprises administering to the patient about 90 mg of the compound of Formula (I) or pharmaceutically acceptable salt thereof per day. In some embodiments, the method comprises administering to the patient about 95 mg of the compound of Formula (I) or pharmaceutically acceptable salt thereof per day. In some embodiments, the method comprises administering to the patient about 100 mg of the compound of Formula (I) or pharmaceutically acceptable salt thereof per day. In some embodiments, the method comprises administering to the patient about 105 mg of the compound of Formula (I) or pharmaceutically acceptable salt thereof per day. In some embodiments, the method comprises administering to the patient about 110 mg of the compound of Formula (I) or pharmaceutically acceptable salt thereof per day. In some embodiments, the method comprises administering to the patient about 115 mg of the compound of Formula (I) or pharmaceutically acceptable salt thereof per day. In some embodiments, the method comprises administering to the patient about 120 mg of the compound of Formula (I) or pharmaceutically acceptable salt thereof per day. In some embodiments, the method comprises administering to the patient about 125 mg of the compound of Formula (I) or pharmaceutically acceptable salt thereof per day. In some embodiments, the method comprises administering to the patient about 130 mg of the compound of Formula (I) or pharmaceutically acceptable salt thereof per day. In some embodiments, the method comprises administering to the patient about 135 mg of the compound of Formula (I) or pharmaceutically acceptable salt thereof per day. In some embodiments, the method comprises administering to the patient about 140 mg of the compound of Formula (I) or pharmaceutically acceptable salt thereof per day. In some embodiments, the method comprises administering to the patient about 145 mg of the compound of Formula (I) or pharmaceutically acceptable salt thereof per day. In some embodiments, the method comprises administering to the patient about 150 mg of the compound of Formula (I) or pharmaceutically acceptable salt thereof per day. In some embodiments, the method comprises administering to the patient about 155 mg of the compound of Formula (I) or pharmaceutically acceptable salt thereof per day. In some embodiments, the method comprises administering to the patient about 160 mg of the compound of Formula (I) or pharmaceutically acceptable salt thereof per day. In some embodiments, the method comprises administering to the patient about 165 mg of the compound of Formula (I) or pharmaceutically acceptable salt thereof per day. In some embodiments, the method comprises administering to the patient about 170 mg of the compound of Formula (I) or pharmaceutically acceptable salt thereof per day. In some embodiments, the method comprises administering to the patient about 175 mg of the compound of Formula (I) or pharmaceutically acceptable salt thereof per day. In some embodiments, the method comprises administering to the patient about 180 mg of the compound of Formula (I) or pharmaceutically acceptable salt thereof per day. In some embodiments, the method comprises administering to the patient about 185 mg of the compound of Formula (I) or pharmaceutically acceptable salt thereof per day. In some embodiments, the method comprises administering to the patient about 190 mg of the compound of Formula (I) or pharmaceutically acceptable salt thereof per day. In some embodiments, the method comprises administering to the patient about 195 mg of the compound of Formula (I) or pharmaceutically acceptable salt thereof per day. In some embodiments, the method comprises administering to the patient about 200 mg of the compound of Formula (I) or pharmaceutically acceptable salt thereof per day.

In some embodiments, the method comprises administering to the patient about 50 mg to about 200 mg of the compound of Formula (I) or pharmaceutically acceptable salt thereof once per day. In some embodiments, the method comprises administering to the patient about 50 mg to about 150 mg of the compound of Formula (I) or pharmaceutically acceptable salt thereof once per day. In some embodiments, the method comprises administering to the patient about 50 mg to about 100 mg of the compound of Formula (I) or pharmaceutically acceptable salt thereof once per day. In some embodiments, the method comprises administering to the patient about 60 mg to about 200 mg of the compound of Formula (I) or pharmaceutically acceptable salt thereof once per day. In some embodiments, the method comprises administering to the patient about 60 mg to about 150 mg of the compound of Formula (I) or pharmaceutically acceptable salt thereof once per day. In some embodiments, the method comprises administering to the patient about 60 mg to about 100 mg of the compound of Formula (I) or pharmaceutically acceptable salt thereof once per day. In some embodiments, the method comprises administering to the patient about 70 mg to about 200 mg of the compound of Formula (I) or pharmaceutically acceptable salt thereof once per day. In some embodiments, the method comprises administering to the patient about 70 mg to about 150 mg of the compound of Formula (I) or pharmaceutically acceptable salt thereof once per day. In some embodiments, the method comprises administering to the patient about 70 mg to about 100 mg of the compound of Formula (I) or pharmaceutically acceptable salt thereof once per day. In some embodiments, the method comprises administering to the patient about 80 mg to about 200 mg of the compound of Formula (I) or pharmaceutically acceptable salt thereof once per day. In some embodiments, the method comprises administering to the patient about 80 mg to about 150 mg of the compound of Formula (I) or pharmaceutically acceptable salt thereof once per day. In some embodiments, the method comprises administering to the patient about 80 mg to about 100 mg of the compound of Formula (I) or pharmaceutically acceptable salt thereof once per day. In some embodiments, the method comprises administering to the patient about 90 mg to about 200 mg of the compound of Formula (I) or pharmaceutically acceptable salt thereof once per day. In some embodiments, the method comprises administering to the patient about 90 mg to about 150 mg of the compound of Formula (I) or pharmaceutically acceptable salt thereof once per day. In some embodiments, the method comprises administering to the patient about 90 mg to about 100 mg of the compound of Formula (I) or pharmaceutically acceptable salt thereof once per day. In some embodiments, the method comprises administering to the patient about 100 mg to about 200 mg of the compound of Formula (I) or pharmaceutically acceptable salt thereof once per day. In some embodiments, the method comprises administering to the patient about 100 mg to about 150 mg of the compound of Formula (I) or pharmaceutically acceptable salt thereof once per day.

In some embodiments, the method comprises administering to the patient about 50 mg of the compound of Formula (I) or pharmaceutically acceptable salt thereof once per day. In some embodiments, the method comprises administering to the patient about 55 mg of the compound of Formula (I) or pharmaceutically acceptable salt thereof once per day. In some embodiments, the method comprises administering to the patient about 60 mg of the compound of Formula (I) or pharmaceutically acceptable salt thereof once per day. In some embodiments, the method comprises administering to the patient about 65 mg of the compound of Formula (I) or pharmaceutically acceptable salt thereof once per day. In some embodiments, the method comprises administering to the patient about 70 mg of the compound of Formula (I) or pharmaceutically acceptable salt thereof once per day. In some embodiments, the method comprises administering to the patient about 75 mg of the compound of Formula (I) or pharmaceutically acceptable salt thereof once per day. In some embodiments, the method comprises administering to the patient about 80 mg of the compound of Formula (I) or pharmaceutically acceptable salt thereof once per day. In some embodiments, the method comprises administering to the patient about 85 mg of the compound of Formula (I) or pharmaceutically acceptable salt thereof once per day. In some embodiments, the method comprises administering to the patient about 90 mg of the compound of Formula (I) or pharmaceutically acceptable salt thereof once per day. In some embodiments, the method comprises administering to the patient about 95 mg of the compound of Formula (I) or pharmaceutically acceptable salt thereof once per day. In some embodiments, the method comprises administering to the patient about 100 mg of the compound of Formula (I) or pharmaceutically acceptable salt thereof once per day. In some embodiments, the method comprises administering to the patient about 105 mg of the compound of Formula (I) or pharmaceutically acceptable salt thereof once per day. In some embodiments, the method comprises administering to the patient about 110 mg of the compound of Formula (I) or pharmaceutically acceptable salt thereof once per day. In some embodiments, the method comprises administering to the patient about 115 mg of the compound of Formula (I) or pharmaceutically acceptable salt thereof once per day. In some embodiments, the method comprises administering to the patient about 120 mg of the compound of Formula (I) or pharmaceutically acceptable salt thereof once per day. In some embodiments, the method comprises administering to the patient about 125 mg of the compound of Formula (I) or pharmaceutically acceptable salt thereof once per day. In some embodiments, the method comprises administering to the patient about 130 mg of the compound of Formula (I) or pharmaceutically acceptable salt thereof once per day. In some embodiments, the method comprises administering to the patient about 135 mg of the compound of Formula (I) or pharmaceutically acceptable salt thereof once per day. In some embodiments, the method comprises administering to the patient about 140 mg of the compound of Formula (I) or pharmaceutically acceptable salt thereof once per day. In some embodiments, the method comprises administering to the patient about 145 mg of the compound of Formula (I) or pharmaceutically acceptable salt thereof once per day. In some embodiments, the method comprises administering to the patient about 150 mg of the compound of Formula (I) or pharmaceutically acceptable salt thereof once per day. In some embodiments, the method comprises administering to the patient about 155 mg of the compound of Formula (I) or pharmaceutically acceptable salt thereof once per day. In some embodiments, the method comprises administering to the patient about 160 mg of the compound of Formula (I) or pharmaceutically acceptable salt thereof once per day. In some embodiments, the method comprises administering to the patient about 165 mg of the compound of Formula (I) or pharmaceutically acceptable salt thereof once per day. In some embodiments, the method comprises administering to the patient about 170 mg of the compound of Formula (I) or pharmaceutically acceptable salt thereof once per day. In some embodiments, the method comprises administering to the patient about 175 mg of the compound of Formula (I) or pharmaceutically acceptable salt thereof once per day. In some embodiments, the method comprises administering to the patient about 180 mg of the compound of Formula (I) or pharmaceutically acceptable salt thereof once per day. In some embodiments, the method comprises administering to the patient about 185 mg of the compound of Formula (I) or pharmaceutically acceptable salt thereof once per day. In some embodiments, the method comprises administering to the patient about 190 mg of the compound of Formula (I) or pharmaceutically acceptable salt thereof once per day. In some embodiments, the method comprises administering to the patient about 195 mg of the compound of Formula (I) or pharmaceutically acceptable salt thereof once per day. In some embodiments, the method comprises administering to the patient about 200 mg of the compound of Formula (I) or pharmaceutically acceptable salt thereof once per day.

In some embodiments, the method comprises administering to the patient about 50 mg to about 100 mg of the compound of Formula (I) or pharmaceutically acceptable salt thereof twice per day. In some embodiments, the method comprises administering to the patient about 60 mg to about 100 mg of the compound of Formula (I) or pharmaceutically acceptable salt thereof twice per day. In some embodiments, the method comprises administering to the patient about 70 mg to about 100 mg of the compound of Formula (I) or pharmaceutically acceptable salt thereof twice per day. In some embodiments, the method comprises administering to the patient about 80 mg to about 100 mg of the compound of Formula (I) or pharmaceutically acceptable salt thereof twice per day. In some embodiments, the method comprises administering to the patient about 90 mg to about 100 mg of the compound of Formula (I) or pharmaceutically acceptable salt thereof twice per day.

In some embodiments, the method comprises administering to the patient about 50 mg of the compound of Formula (I) or pharmaceutically acceptable salt thereof twice per day. In some embodiments, the method comprises administering to the patient about 55 mg of the compound of Formula (I) or pharmaceutically acceptable salt thereof twice per day. In some embodiments, the method comprises administering to the patient about 60 mg of the compound of Formula (I) or pharmaceutically acceptable salt thereof twice per day. In some embodiments, the method comprises administering to the patient about 65 mg of the compound of Formula (I) or pharmaceutically acceptable salt thereof twice per day. In some embodiments, the method comprises administering to the patient about 70 mg of the compound of Formula (I) or pharmaceutically acceptable salt thereof twice per day. In some embodiments, the method comprises administering to the patient about 75 mg of the compound of Formula (I) or pharmaceutically acceptable salt thereof twice per day. In some embodiments, the method comprises administering to the patient about 80 mg of the compound of Formula (I) or pharmaceutically acceptable salt thereof twice per day. In some embodiments, the method comprises administering to the patient about 85 mg of the compound of Formula (I) or pharmaceutically acceptable salt thereof twice per day. In some embodiments, the method comprises administering to the patient about 90 mg of the compound of Formula (I) or pharmaceutically acceptable salt thereof twice per day. In some embodiments, the method comprises administering to the patient about 95 mg of the compound of Formula (I) or pharmaceutically acceptable salt thereof twice per day. In some embodiments, the method comprises administering to the patient about 100 mg of the compound of Formula (I) or pharmaceutically acceptable salt thereof twice per day.

The compound of Formula (I) or pharmaceutically acceptable salt thereof may, in another embodiment, be administered to the patient on an intermittent basis in the methods of achieving at least a partial response in a patient having mesothelioma described herein. In some embodiments, the compound of Formula (I) or pharmaceutically acceptable salt thereof is administered the patient every other day. In some embodiments, the compound of Formula (I) or pharmaceutically acceptable salt thereof is administered the patient every other day. In some embodiments, the compound of Formula (I) or pharmaceutically acceptable salt thereof is administered the patient once per week. In some embodiments, the compound of Formula (I) or pharmaceutically acceptable salt thereof is administered the patient twice per week. In some embodiments, the compound of Formula (I) or pharmaceutically acceptable salt thereof is administered the patient three times per week.

In some embodiments, the method comprises administering to the patient about 50 mg to about 200 mg of the compound of Formula (I) or pharmaceutically acceptable salt thereof once a week. In some embodiments, the method comprises administering to the patient about 50 mg to about 150 mg of the compound of Formula (I) or pharmaceutically acceptable salt thereof once a week. In some embodiments, the method comprises administering to the patient about 50 mg to about 100 mg of the compound of Formula (I) or pharmaceutically acceptable salt thereof once a week. In some embodiments, the method comprises administering to the patient about 60 mg to about 200 mg of the compound of Formula (I) or pharmaceutically acceptable salt thereof once a week. In some embodiments, the method comprises administering to the patient about 60 mg to about 150 mg of the compound of Formula (I) or pharmaceutically acceptable salt thereof once a week. In some embodiments, the method comprises administering to the patient about 60 mg to about 100 mg of the compound of Formula (I) or pharmaceutically acceptable salt thereof once a week. In some embodiments, the method comprises administering to the patient about 70 mg to about 200 mg of the compound of Formula (I) or pharmaceutically acceptable salt thereof once a week. In some embodiments, the method comprises administering to the patient about 70 mg to about 150 mg of the compound of Formula (I) or pharmaceutically acceptable salt thereof once a week. In some embodiments, the method comprises administering to the patient about 70 mg to about 100 mg of the compound of Formula (I) or pharmaceutically acceptable salt thereof once a week. In some embodiments, the method comprises administering to the patient about 80 mg to about 200 mg of the compound of Formula (I) or pharmaceutically acceptable salt thereof once a week. In some embodiments, the method comprises administering to the patient about 80 mg to about 150 mg of the compound of Formula (I) or pharmaceutically acceptable salt thereof once a week. In some embodiments, the method comprises administering to the patient about 80 mg to about 100 mg of the compound of Formula (I) or pharmaceutically acceptable salt thereof once a week. In some embodiments, the method comprises administering to the patient about 90 mg to about 200 mg of the compound of Formula (I) or pharmaceutically acceptable salt thereof once a week. In some embodiments, the method comprises administering to the patient about 90 mg to about 150 mg of the compound of Formula (I) or pharmaceutically acceptable salt thereof once a week. In some embodiments, the method comprises administering to the patient about 90 mg to about 100 mg of the compound of Formula (I) or pharmaceutically acceptable salt thereof once a week. In some embodiments, the method comprises administering to the patient about 100 mg to about 200 mg of the compound of Formula (I) or pharmaceutically acceptable salt thereof once a week. In some embodiments, the method comprises administering to the patient about 100 mg to about 150 mg of the compound of Formula (I) or pharmaceutically acceptable salt thereof once a week.

In some embodiments, the method comprises administering to the patient about 50 mg of the compound of Formula (I) or pharmaceutically acceptable salt thereof once a week. In some embodiments, the method comprises administering to the patient about 55 mg of the compound of Formula (I) or pharmaceutically acceptable salt thereof once a week. In some embodiments, the method comprises administering to the patient about 60 mg of the compound of Formula (I) or pharmaceutically acceptable salt thereof once a week. In some embodiments, the method comprises administering to the patient about 65 mg of the compound of Formula (I) or pharmaceutically acceptable salt thereof once a week. In some embodiments, the method comprises administering to the patient about 70 mg of the compound of Formula (I) or pharmaceutically acceptable salt thereof once a week. In some embodiments, the method comprises administering to the patient about 75 mg of the compound of Formula (I) or pharmaceutically acceptable salt thereof once a week. In some embodiments, the method comprises administering to the patient about 80 mg of the compound of Formula (I) or pharmaceutically acceptable salt thereof once a week. In some embodiments, the method comprises administering to the patient about 85 mg of the compound of Formula (I) or pharmaceutically acceptable salt thereof once a week. In some embodiments, the method comprises administering to the patient about 90 mg of the compound of Formula (I) or pharmaceutically acceptable salt thereof once a week. In some embodiments, the method comprises administering to the patient about 95 mg of the compound of Formula (I) or pharmaceutically acceptable salt thereof once a week. In some embodiments, the method comprises administering to the patient about 100 mg of the compound of Formula (I) or pharmaceutically acceptable salt thereof once a week. In some embodiments, the method comprises administering to the patient about 105 mg of the compound of Formula (I) or pharmaceutically acceptable salt thereof once a week. In some embodiments, the method comprises administering to the patient about 110 mg of the compound of Formula (I) or pharmaceutically acceptable salt thereof once a week. In some embodiments, the method comprises administering to the patient about 115 mg of the compound of Formula (I) or pharmaceutically acceptable salt thereof once a week. In some embodiments, the method comprises administering to the patient about 120 mg of the compound of Formula (I) or pharmaceutically acceptable salt thereof once a week. In some embodiments, the method comprises administering to the patient about 125 mg of the compound of Formula (I) or pharmaceutically acceptable salt thereof once a week. In some embodiments, the method comprises administering to the patient about 130 mg of the compound of Formula (I) or pharmaceutically acceptable salt thereof once a week. In some embodiments, the method comprises administering to the patient about 135 mg of the compound of Formula (I) or pharmaceutically acceptable salt thereof once a week. In some embodiments, the method comprises administering to the patient about 140 mg of the compound of Formula (I) or pharmaceutically acceptable salt thereof once a week. In some embodiments, the method comprises administering to the patient about 145 mg of the compound of Formula (I) or pharmaceutically acceptable salt thereof once a week. In some embodiments, the method comprises administering to the patient about 150 mg of the compound of Formula (I) or pharmaceutically acceptable salt thereof once a week. In some embodiments, the method comprises administering to the patient about 155 mg of the compound of Formula (I) or pharmaceutically acceptable salt thereof once a week. In some embodiments, the method comprises administering to the patient about 160 mg of the compound of Formula (I) or pharmaceutically acceptable salt thereof once a week. In some embodiments, the method comprises administering to the patient about 165 mg of the compound of Formula (I) or pharmaceutically acceptable salt thereof once a week. In some embodiments, the method comprises administering to the patient about 170 mg of the compound of Formula (I) or pharmaceutically acceptable salt thereof once a week. In some embodiments, the method comprises administering to the patient about 175 mg of the compound of Formula (I) or pharmaceutically acceptable salt thereof once a week. In some embodiments, the method comprises administering to the patient about 180 mg of the compound of Formula (I) or pharmaceutically acceptable salt thereof once a week. In some embodiments, the method comprises administering to the patient about 185 mg of the compound of Formula (I) or pharmaceutically acceptable salt thereof once a week. In some embodiments, the method comprises administering to the patient about 190 mg of the compound of Formula (I) or pharmaceutically acceptable salt thereof once a week. In some embodiments, the method comprises administering to the patient about 195 mg of the compound of Formula (I) or pharmaceutically acceptable salt thereof once a week. In some embodiments, the method comprises administering to the patient about 200 mg of the compound of Formula (I) or pharmaceutically acceptable salt thereof once a week.

In some embodiments, the method comprises administering to the patient about 50 mg to about 200 mg of the compound of Formula (I) or pharmaceutically acceptable salt thereof twice a week. In some embodiments, the method comprises administering to the patient about 50 mg to about 150 mg of the compound of Formula (I) or pharmaceutically acceptable salt thereof twice a week. In some embodiments, the method comprises administering to the patient about 50 mg to about 100 mg of the compound of Formula (I) or pharmaceutically acceptable salt thereof twice a week. In some embodiments, the method comprises administering to the patient about 60 mg to about 200 mg of the compound of Formula (I) or pharmaceutically acceptable salt thereof twice a week. In some embodiments, the method comprises administering to the patient about 60 mg to about 150 mg of the compound of Formula (I) or pharmaceutically acceptable salt thereof twice a week. In some embodiments, the method comprises administering to the patient about 60 mg to about 100 mg of the compound of Formula (I) or pharmaceutically acceptable salt thereof twice a week. In some embodiments, the method comprises administering to the patient about 70 mg to about 200 mg of the compound of Formula (I) or pharmaceutically acceptable salt thereof twice a week. In some embodiments, the method comprises administering to the patient about 70 mg to about 150 mg of the compound of Formula (I) or pharmaceutically acceptable salt thereof twice a week. In some embodiments, the method comprises administering to the patient about 70 mg to about 100 mg of the compound of Formula (I) or pharmaceutically acceptable salt thereof twice a week. In some embodiments, the method comprises administering to the patient about 80 mg to about 200 mg of the compound of Formula (I) or pharmaceutically acceptable salt thereof twice a week. In some embodiments, the method comprises administering to the patient about 80 mg to about 150 mg of the compound of Formula (I) or pharmaceutically acceptable salt thereof twice a week. In some embodiments, the method comprises administering to the patient about 80 mg to about 100 mg of the compound of Formula (I) or pharmaceutically acceptable salt thereof twice a week. In some embodiments, the method comprises administering to the patient about 90 mg to about 200 mg of the compound of Formula (I) or pharmaceutically acceptable salt thereof twice a week. In some embodiments, the method comprises administering to the patient about 90 mg to about 150 mg of the compound of Formula (I) or pharmaceutically acceptable salt thereof twice a week. In some embodiments, the method comprises administering to the patient about 90 mg to about 100 mg of the compound of Formula (I) or pharmaceutically acceptable salt thereof twice a week. In some embodiments, the method comprises administering to the patient about 100 mg to about 200 mg of the compound of Formula (I) or pharmaceutically acceptable salt thereof twice a week. In some embodiments, the method comprises administering to the patient about 100 mg to about 150 mg of the compound of Formula (I) or pharmaceutically acceptable salt thereof twice a week.

In some embodiments, the method comprises administering to the patient about 50 mg of the compound of Formula (I) or pharmaceutically acceptable salt thereof twice a week. In some embodiments, the method comprises administering to the patient about 55 mg of the compound of Formula (I) or pharmaceutically acceptable salt thereof twice a week. In some embodiments, the method comprises administering to the patient about 60 mg of the compound of Formula (I) or pharmaceutically acceptable salt thereof twice a week. In some embodiments, the method comprises administering to the patient about 65 mg of the compound of Formula (I) or pharmaceutically acceptable salt thereof twice a week. In some embodiments, the method comprises administering to the patient about 70 mg of the compound of Formula (I) or pharmaceutically acceptable salt thereof twice a week. In some embodiments, the method comprises administering to the patient about 75 mg of the compound of Formula (I) or pharmaceutically acceptable salt thereof twice a week. In some embodiments, the method comprises administering to the patient about 80 mg of the compound of Formula (I) or pharmaceutically acceptable salt thereof twice a week. In some embodiments, the method comprises administering to the patient about 85 mg of the compound of Formula (I) or pharmaceutically acceptable salt thereof twice a week. In some embodiments, the method comprises administering to the patient about 90 mg of the compound of Formula (I) or pharmaceutically acceptable salt thereof twice a week. In some embodiments, the method comprises administering to the patient about 95 mg of the compound of Formula (I) or pharmaceutically acceptable salt thereof twice a week. In some embodiments, the method comprises administering to the patient about 100 mg of the compound of Formula (I) or pharmaceutically acceptable salt thereof twice a week. In some embodiments, the method comprises administering to the patient about 105 mg of the compound of Formula (I) or pharmaceutically acceptable salt thereof twice a week. In some embodiments, the method comprises administering to the patient about 110 mg of the compound of Formula (I) or pharmaceutically acceptable salt thereof twice a week. In some embodiments, the method comprises administering to the patient about 115 mg of the compound of Formula (I) or pharmaceutically acceptable salt thereof twice a week. In some embodiments, the method comprises administering to the patient about 120 mg of the compound of Formula (I) or pharmaceutically acceptable salt thereof twice a week. In some embodiments, the method comprises administering to the patient about 125 mg of the compound of Formula (I) or pharmaceutically acceptable salt thereof twice a week. In some embodiments, the method comprises administering to the patient about 130 mg of the compound of Formula (I) or pharmaceutically acceptable salt thereof twice a week. In some embodiments, the method comprises administering to the patient about 135 mg of the compound of Formula (I) or pharmaceutically acceptable salt thereof twice a week. In some embodiments, the method comprises administering to the patient about 140 mg of the compound of Formula (I) or pharmaceutically acceptable salt thereof twice a week. In some embodiments, the method comprises administering to the patient about 145 mg of the compound of Formula (I) or pharmaceutically acceptable salt thereof twice a week. In some embodiments, the method comprises administering to the patient about 150 mg of the compound of Formula (I) or pharmaceutically acceptable salt thereof twice a week. In some embodiments, the method comprises administering to the patient about 155 mg of the compound of Formula (I) or pharmaceutically acceptable salt thereof twice a week. In some embodiments, the method comprises administering to the patient about 160 mg of the compound of Formula (I) or pharmaceutically acceptable salt thereof twice a week. In some embodiments, the method comprises administering to the patient about 165 mg of the compound of Formula (I) or pharmaceutically acceptable salt thereof twice a week. In some embodiments, the method comprises administering to the patient about 170 mg of the compound of Formula (I) or pharmaceutically acceptable salt thereof twice a week. In some embodiments, the method comprises administering to the patient about 175 mg of the compound of Formula (I) or pharmaceutically acceptable salt thereof twice a week. In some embodiments, the method comprises administering to the patient about 180 mg of the compound of Formula (I) or pharmaceutically acceptable salt thereof twice a week. In some embodiments, the method comprises administering to the patient about 185 mg of the compound of Formula (I) or pharmaceutically acceptable salt thereof twice a week. In some embodiments, the method comprises administering to the patient about 190 mg of the compound of Formula (I) or pharmaceutically acceptable salt thereof twice a week. In some embodiments, the method comprises administering to the patient about 195 mg of the compound of Formula (I) or pharmaceutically acceptable salt thereof twice a week. In some embodiments, the method comprises administering to the patient about 200 mg of the compound of Formula (I) or pharmaceutically acceptable salt thereof twice a week.

In some embodiments, the method comprises administering to the patient about 50 mg to about 200 mg of the compound of Formula (I) or pharmaceutically acceptable salt thereof three times a week. In some embodiments, the method comprises administering to the patient about 50 mg to about 150 mg of the compound of Formula (I) or pharmaceutically acceptable salt thereof three times a week. In some embodiments, the method comprises administering to the patient about 50 mg to about 100 mg of the compound of Formula (I) or pharmaceutically acceptable salt thereof three times a week. In some embodiments, the method comprises administering to the patient about 60 mg to about 200 mg of the compound of Formula (I) or pharmaceutically acceptable salt thereof three times a week. In some embodiments, the method comprises administering to the patient about 60 mg to about 150 mg of the compound of Formula (I) or pharmaceutically acceptable salt thereof three times a week. In some embodiments, the method comprises administering to the patient about 60 mg to about 100 mg of the compound of Formula (I) or pharmaceutically acceptable salt thereof three times a week. In some embodiments, the method comprises administering to the patient about 70 mg to about 200 mg of the compound of Formula (I) or pharmaceutically acceptable salt thereof three times a week. In some embodiments, the method comprises administering to the patient about 70 mg to about 150 mg of the compound of Formula (I) or pharmaceutically acceptable salt thereof three times a week. In some embodiments, the method comprises administering to the patient about 70 mg to about 100 mg of the compound of Formula (I) or pharmaceutically acceptable salt thereof three times a week. In some embodiments, the method comprises administering to the patient about 80 mg to about 200 mg of the compound of Formula (I) or pharmaceutically acceptable salt thereof three times a week. In some embodiments, the method comprises administering to the patient about 80 mg to about 150 mg of the compound of Formula (I) or pharmaceutically acceptable salt thereof three times a week. In some embodiments, the method comprises administering to the patient about 80 mg to about 100 mg of the compound of Formula (I) or pharmaceutically acceptable salt thereof three times a week. In some embodiments, the method comprises administering to the patient about 90 mg to about 200 mg of the compound of Formula (I) or pharmaceutically acceptable salt thereof three times a week. In some embodiments, the method comprises administering to the patient about 90 mg to about 150 mg of the compound of Formula (I) or pharmaceutically acceptable salt thereof three times a week. In some embodiments, the method comprises administering to the patient about 90 mg to about 100 mg of the compound of Formula (I) or pharmaceutically acceptable salt thereof three times a week. In some embodiments, the method comprises administering to the patient about 100 mg to about 200 mg of the compound of Formula (I) or pharmaceutically acceptable salt thereof three times a week. In some embodiments, the method comprises administering to the patient about 100 mg to about 150 mg of the compound of Formula (I) or pharmaceutically acceptable salt thereof three times a week.

In some embodiments, the method comprises administering to the patient about 50 mg of the compound of Formula (I) or pharmaceutically acceptable salt thereof three times a week. In some embodiments, the method comprises administering to the patient about 55 mg of the compound of Formula (I) or pharmaceutically acceptable salt thereof three times a week. In some embodiments, the method comprises administering to the patient about 60 mg of the compound of Formula (I) or pharmaceutically acceptable salt thereof three times a week. In some embodiments, the method comprises administering to the patient about 65 mg of the compound of Formula (I) or pharmaceutically acceptable salt thereof three times a week. In some embodiments, the method comprises administering to the patient about 70 mg of the compound of Formula (I) or pharmaceutically acceptable salt thereof three times a week. In some embodiments, the method comprises administering to the patient about 75 mg of the compound of Formula (I) or pharmaceutically acceptable salt thereof three times a week. In some embodiments, the method comprises administering to the patient about 80 mg of the compound of Formula (I) or pharmaceutically acceptable salt thereof three times a week. In some embodiments, the method comprises administering to the patient about 85 mg of the compound of Formula (I) or pharmaceutically acceptable salt thereof three times a week. In some embodiments, the method comprises administering to the patient about 90 mg of the compound of Formula (I) or pharmaceutically acceptable salt thereof three times a week. In some embodiments, the method comprises administering to the patient about 95 mg of the compound of Formula (I) or pharmaceutically acceptable salt thereof three times a week. In some embodiments, the method comprises administering to the patient about 100 mg of the compound of Formula (I) or pharmaceutically acceptable salt thereof three times a week. In some embodiments, the method comprises administering to the patient about 105 mg of the compound of Formula (I) or pharmaceutically acceptable salt thereof three times a week. In some embodiments, the method comprises administering to the patient about 110 mg of the compound of Formula (I) or pharmaceutically acceptable salt thereof three times a week. In some embodiments, the method comprises administering to the patient about 115 mg of the compound of Formula (I) or pharmaceutically acceptable salt thereof three times a week. In some embodiments, the method comprises administering to the patient about 120 mg of the compound of Formula (I) or pharmaceutically acceptable salt thereof three times a week. In some embodiments, the method comprises administering to the patient about 125 mg of the compound of Formula (I) or pharmaceutically acceptable salt thereof three times a week. In some embodiments, the method comprises administering to the patient about 130 mg of the compound of Formula (I) or pharmaceutically acceptable salt thereof three times a week. In some embodiments, the method comprises administering to the patient about 135 mg of the compound of Formula (I) or pharmaceutically acceptable salt thereof three times a week. In some embodiments, the method comprises administering to the patient about 140 mg of the compound of Formula (I) or pharmaceutically acceptable salt thereof three times a week. In some embodiments, the method comprises administering to the patient about 145 mg of the compound of Formula (I) or pharmaceutically acceptable salt thereof three times a week. In some embodiments, the method comprises administering to the patient about 150 mg of the compound of Formula (I) or pharmaceutically acceptable salt thereof three times a week. In some embodiments, the method comprises administering to the patient about 155 mg of the compound of Formula (I) or pharmaceutically acceptable salt thereof three times a week. In some embodiments, the method comprises administering to the patient about 160 mg of the compound of Formula (I) or pharmaceutically acceptable salt thereof three times a week. In some embodiments, the method comprises administering to the patient about 165 mg of the compound of Formula (I) or pharmaceutically acceptable salt thereof three times a week. In some embodiments, the method comprises administering to the patient about 170 mg of the compound of Formula (I) or pharmaceutically acceptable salt thereof three times a week. In some embodiments, the method comprises administering to the patient about 175 mg of the compound of Formula (I) or pharmaceutically acceptable salt thereof three times a week. In some embodiments, the method comprises administering to the patient about 180 mg of the compound of Formula (I) or pharmaceutically acceptable salt thereof three times a week. In some embodiments, the method comprises administering to the patient about 185 mg of the compound of Formula (I) or pharmaceutically acceptable salt thereof three times a week. In some embodiments, the method comprises administering to the patient about 190 mg of the compound of Formula (I) or pharmaceutically acceptable salt thereof three times a week. In some embodiments, the method comprises administering to the patient about 195 mg of the compound of Formula (I) or pharmaceutically acceptable salt thereof three times a week. In some embodiments, the method comprises administering to the patient about 200 mg of the compound of Formula (I) or pharmaceutically acceptable salt thereof three times a week.

In some embodiments, the method comprises administering to the patient about 50 mg to about 200 mg of the compound of Formula (I) or pharmaceutically acceptable salt thereof every other day. In some embodiments, the method comprises administering to the patient about 50 mg to about 150 mg of the compound of Formula (I) or pharmaceutically acceptable salt thereof every other day. In some embodiments, the method comprises administering to the patient about 50 mg to about 100 mg of the compound of Formula (I) or pharmaceutically acceptable salt thereof every other day. In some embodiments, the method comprises administering to the patient about 60 mg to about 200 mg of the compound of Formula (I) or pharmaceutically acceptable salt thereof every other day. In some embodiments, the method comprises administering to the patient about 60 mg to about 150 mg of the compound of Formula (I) or pharmaceutically acceptable salt thereof every other day. In some embodiments, the method comprises administering to the patient about 60 mg to about 100 mg of the compound of Formula (I) or pharmaceutically acceptable salt thereof every other day. In some embodiments, the method comprises administering to the patient about 70 mg to about 200 mg of the compound of Formula (I) or pharmaceutically acceptable salt thereof every other day. In some embodiments, the method comprises administering to the patient about 70 mg to about 150 mg of the compound of Formula (I) or pharmaceutically acceptable salt thereof every other day. In some embodiments, the method comprises administering to the patient about 70 mg to about 100 mg of the compound of Formula (I) or pharmaceutically acceptable salt thereof every other day. In some embodiments, the method comprises administering to the patient about 80 mg to about 200 mg of the compound of Formula (I) or pharmaceutically acceptable salt thereof every other day. In some embodiments, the method comprises administering to the patient about 80 mg to about 150 mg of the compound of Formula (I) or pharmaceutically acceptable salt thereof every other day. In some embodiments, the method comprises administering to the patient about 80 mg to about 100 mg of the compound of Formula (I) or pharmaceutically acceptable salt thereof every other day. In some embodiments, the method comprises administering to the patient about 90 mg to about 200 mg of the compound of Formula (I) or pharmaceutically acceptable salt thereof every other day. In some embodiments, the method comprises administering to the patient about 90 mg to about 150 mg of the compound of Formula (I) or pharmaceutically acceptable salt thereof every other day. In some embodiments, the method comprises administering to the patient about 90 mg to about 100 mg of the compound of Formula (I) or pharmaceutically acceptable salt thereof every other day. In some embodiments, the method comprises administering to the patient about 100 mg to about 200 mg of the compound of Formula (I) or pharmaceutically acceptable salt thereof every other day. In some embodiments, the method comprises administering to the patient about 100 mg to about 150 mg of the compound of Formula (I) or pharmaceutically acceptable salt thereof every other day.

In some embodiments, the method comprises administering to the patient about 50 mg of the compound of Formula (I) or pharmaceutically acceptable salt thereof every other day. In some embodiments, the method comprises administering to the patient about 55 mg of the compound of Formula (I) or pharmaceutically acceptable salt thereof every other day. In some embodiments, the method comprises administering to the patient about 60 mg of the compound of Formula (I) or pharmaceutically acceptable salt thereof every other day. In some embodiments, the method comprises administering to the patient about 65 mg of the compound of Formula (I) or pharmaceutically acceptable salt thereof every other day. In some embodiments, the method comprises administering to the patient about 70 mg of the compound of Formula (I) or pharmaceutically acceptable salt thereof every other day. In some embodiments, the method comprises administering to the patient about 75 mg of the compound of Formula (I) or pharmaceutically acceptable salt thereof every other day. In some embodiments, the method comprises administering to the patient about 80 mg of the compound of Formula (I) or pharmaceutically acceptable salt thereof every other day. In some embodiments, the method comprises administering to the patient about 85 mg of the compound of Formula (I) or pharmaceutically acceptable salt thereof every other day. In some embodiments, the method comprises administering to the patient about 90 mg of the compound of Formula (I) or pharmaceutically acceptable salt thereof every other day. In some embodiments, the method comprises administering to the patient about 95 mg of the compound of Formula (I) or pharmaceutically acceptable salt thereof every other day. In some embodiments, the method comprises administering to the patient about 100 mg of the compound of Formula (I) or pharmaceutically acceptable salt thereof every other day. In some embodiments, the method comprises administering to the patient about 105 mg of the compound of Formula (I) or pharmaceutically acceptable salt thereof every other day. In some embodiments, the method comprises administering to the patient about 110 mg of the compound of Formula (I) or pharmaceutically acceptable salt thereof every other day. In some embodiments, the method comprises administering to the patient about 115 mg of the compound embodiments, the method comprises administering to the patient about 120 mg of the compound of Formula (I) or pharmaceutically acceptable salt thereof every other day. In some embodiments, the method comprises administering to the patient about 125 mg of the compound of Formula (I) or pharmaceutically acceptable salt thereof every other day. In some embodiments, the method comprises administering to the patient about 130 mg of the compound of Formula (I) or pharmaceutically acceptable salt thereof every other day. In some embodiments, the method comprises administering to the patient about 135 mg of the compound of Formula (I) or pharmaceutically acceptable salt thereof every other day. In some embodiments, the method comprises administering to the patient about 140 mg of the compound of Formula (I) or pharmaceutically acceptable salt thereof every other day. In some embodiments, the method comprises administering to the patient about 145 mg of the compound of Formula (I) or pharmaceutically acceptable salt thereof every other day. In some embodiments, the method comprises administering to the patient about 150 mg of the compound of Formula (I) or pharmaceutically acceptable salt thereof every other day. In some embodiments, the method comprises administering to the patient about 155 mg of the compound of Formula (I) or pharmaceutically acceptable salt thereof every other day. In some embodiments, the method comprises administering to the patient about 160 mg of the compound of Formula (I) or pharmaceutically acceptable salt thereof every other day. In some embodiments, the method comprises administering to the patient about 165 mg of the compound of Formula (I) or pharmaceutically acceptable salt thereof every other day. In some embodiments, the method comprises administering to the patient about 170 mg of the compound of Formula (I) or pharmaceutically acceptable salt thereof every other day. In some embodiments, the method comprises administering to the patient about 175 mg of the compound of Formula (I) or pharmaceutically acceptable salt thereof every other day. In some embodiments, the method comprises administering to the patient about 180 mg of the compound of Formula (I) or pharmaceutically acceptable salt thereof every other day. In some embodiments, the method comprises administering to the patient about 185 mg of the compound of Formula (I) or pharmaceutically acceptable salt thereof every other day. In some embodiments, the method comprises administering to the patient about 190 mg of the compound of Formula (I) or pharmaceutically acceptable salt thereof every other day. In some embodiments, the method comprises administering to the patient about 195 mg of the compound of Formula (I) or pharmaceutically acceptable salt thereof every other day. In some embodiments, the method comprises administering to the patient about 200 mg of the compound of Formula (I) or pharmaceutically acceptable salt thereof every other day.

In some embodiments, the methods further comprise administering a combination agent to the patient in need thereof. In some embodiments, the combination agent is carboplatin. In some embodiments, the combination agent is paclitaxel. In some embodiments, the method further comprises administering pemetrexed to the patient in need thereof. In some embodiments, the method further comprises administering one or more combination agents to the patient in need thereof. In some embodiments, the method further comprises administering carboplatin and paclitaxel.

In some embodiments, the combination agent is intravenously administered to the patient in need thereof. The one or more combination agents administered to a patient in the methods of achieving at least a partial response in patients having mesothelioma described herein can be dosed to achieve various concentrations of the combination agents in the patient's plasma, which can be quantified by the area under the curve (AUC) for each combination agent administered, e.g., intravenously administered. In some embodiments, the combination agent, (e.g., carboplatin) is intravenously administered to the patient in an amount sufficient to provide an AUC of about 5 to about 7 (e.g., an AUC of about 5 or about 6). In some embodiments, the method comprises reducing each of the amount of the compound of Formula (I) or pharmaceutically acceptable salt thereof and the combination agent administered to the patient after one or more months of administering to the patient in need thereof.

In some embodiments, the paclitaxel is intravenously administered to the patient, e.g., by intravenous infusion. The intravenously administered paclitaxel can be administered to a patient at various dose levels. In some embodiments, the paclitaxel is administered to the patient at about 50 mg/m² to about 150 mg/m². In some embodiments, the paclitaxel is administered to the patient at about 50 mg/m² to about 100 mg/m². In some embodiments, the paclitaxel is administered to the patient at about 60 mg/m² to about 80 mg/m². In some embodiments, the paclitaxel is administered to the patient at about 60 mg/m². In some embodiments, the paclitaxel is administered to the patient at about 80 mg/m². In some embodiments, the paclitaxel is administered to the patient at about 135 mg/m². In some embodiments, the paclitaxel is administered to the patient at about 175 mg/m².

Furthermore, the paclitaxel can be administered to the patient at various frequencies. In some embodiments, the paclitaxel is administered to the patient once per week (e.g., at about 60 mg/m² once per week or about 80 mg/m² once per week). In some embodiments, the paclitaxel is administered to the patient once every three weeks (e.g., at about 135 mg/m² once every three weeks or about 175 mg/m² once every three weeks). In some embodiments, the paclitaxel is administered to the patient by intravenous infusion once per week. In some embodiments, the paclitaxel is administered to the patient by intravenous infusion once per week for three consecutive weeks. In some embodiments, the method comprises administering paclitaxel to the patient by intravenous infusion for more than one cycle, wherein each cycle comprises administering paclitaxel to the patient by intravenous infusion once per week for three consecutive weeks (e.g., once per week for three consecutive weeks at about 60 mg/m² or about 80 mg/m²). In some embodiments, the method comprises not administering paclitaxel to the patient for at least one week between each cycle.

In some embodiments, the paclitaxel is administered to the patient by infusion once every three weeks. In some embodiments, the paclitaxel is administered to the patient for about 60 minutes once per week. In some embodiments, the paclitaxel is administered to the patient for about 3 hours once per week. In some embodiments, the paclitaxel is administered to the patient for about 60 minutes once every three weeks. In some embodiments, the paclitaxel is administered to the patient for about 3 hours once every three weeks. In some embodiments, the paclitaxel is administered to the patient by intravenous infusion for about 60 minutes once per week. In some embodiments, the paclitaxel is administered to the patient by infusion for about 3 hours once per week. In some embodiments, the paclitaxel is administered to the patient by intravenous infusion for about 60 minutes once every three weeks. In some embodiments, the paclitaxel is administered to the patient by infusion for about 3 hours once every three weeks. In some embodiments, the paclitaxel is continuously administered to the patient for about 60 minutes once per week. In some embodiments, the paclitaxel is continuously administered to the patient for about 3 hours once per week. In some embodiments, the paclitaxel is continuously administered to the patient for about 60 minutes once every three weeks. In some embodiments, the paclitaxel is continuously administered to the patient for about 3 hours once every three weeks. In some embodiments, the paclitaxel is continuously administered to the patient by infusion for about 60 minutes once per week. In some embodiments, the paclitaxel is continuously administered to the patient by intravenous infusion for about 3 hours once per week. In some embodiments, the paclitaxel is continuously administered to the patient by infusion for about 60 minutes once every three weeks. In some embodiments, the paclitaxel is continuously administered to the patient by intravenous infusion for about 3 hours once every three weeks.

The method of achieving at least a partial response in patients having mesothelioma described herein described herein can further comprise reducing each of the amount of the compound of Formula (I) or pharmaceutically acceptable salt thereof and the combination agent administered to the patient after one or more months of administering said compound of Formula (I) or pharmaceutically acceptable salt thereof and said combination agent to the patient in need thereof.

In embodiments of the methods of achieving at least a partial response in a patient having mesothelioma described herein, the disclosure also contemplates administration of the compound of Formula (I) or a pharmaceutically acceptable salt to a patient in need thereof prior to (neo-adjuvant) or after (adjuvant) surgery (e.g., surgical treatment of a solid tumor). In some embodiments, the compound of Formula (I) or a pharmaceutically acceptable salt is administered as an adjuvant. In some embodiments, the compound of Formula (I) or a pharmaceutically acceptable salt is administered as a neo-adjuvant. In some embodiments, the compound of Formula (I) or a pharmaceutically acceptable salt is administered as a neo-adjuvant and an adjuvant. In some embodiments, the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 100 years. In some embodiments, administration of the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 5 years. In some embodiments, administration of the compound of Formula (I) or a pharmaceutically acceptable salt is administered to a patient in need thereof as a neo-adjuvant for a period of 3 months to 6 months, followed by administration of the compound of Formula (I) or a pharmaceutically acceptable salt as an adjuvant for a period from 1 day to 5 years. In some embodiments, no administration of the compound of Formula (I) or a pharmaceutically acceptable salt occurs prior to surgery. In some embodiments, the compound of Formula (I) or a pharmaceutically acceptable salt is administered as an adjuvant for a period from 1 day to 100 years. In some embodiments, the compound of Formula (I) or a pharmaceutically acceptable salt is administered as an adjuvant for a period from 1 day to 5 years.

In yet another embodiment, provided herein is a method for achieving a stable disease condition in a patient having mesothelioma, comprising to the patient administering the compound of Formula (I)

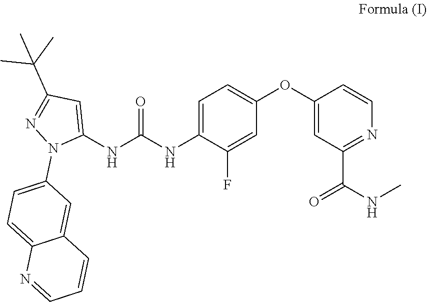 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In some embodiments, the method further comprises administering a combination agent to the patient in need thereof. In some embodiments, the combination agent is carboplatin. In some embodiments, the combination agent is paclitaxel. In some embodiments, the method further comprises administering pemetrexed to the patient in need thereof. In some embodiments, the method further comprises administering one or more combination agents to the patient in need thereof. In some embodiments, the method further comprises administering carboplatin and paclitaxel.

In some embodiments, the patient has previously been administered at least one prior line of therapy. In some embodiments, the therapy is a platinum-based therapy. In some embodiments, the patient previously been administered carboplatin and/or pemetrexed. In some embodiments, the patient previously been administered cisplatin and/or pemetrexed.

In embodiments of the methods for treating mesothelioma described herein, the compound of Formula (I) or pharmaceutically acceptable salt thereof can be orally administered to the patient in need thereof. In certain embodiments, the compound of Formula (I) or pharmaceutically acceptable salt thereof is administered to the patient daily. In some embodiments, the method comprises administering to the patient about 50 mg to about 150 mg of the compound of Formula (I) or pharmaceutically acceptable salt thereof per day. In some embodiments, the method comprises administering to the patient about 50 mg to about 100 mg of the compound of Formula (I) or pharmaceutically acceptable salt thereof per day. In some embodiments, the method comprises administering to the patient about 60 mg to about 200 mg of the compound of Formula (I) or pharmaceutically acceptable salt thereof per day. In some embodiments, the method comprises administering to the patient about 60 mg to about 150 mg of the compound of Formula (I) or pharmaceutically acceptable salt thereof per day. In some embodiments, the method comprises administering to the patient about 60 mg to about 100 mg of the compound of Formula (I) or pharmaceutically acceptable salt thereof per day. In some embodiments, the method comprises administering to the patient about 70 mg to about 200 mg of the compound of Formula (I) or pharmaceutically acceptable salt thereof per day. In some embodiments, the method comprises administering to the patient about 70 mg to about 150 mg of the compound of Formula (I) or pharmaceutically acceptable salt thereof per day. In some embodiments, the method comprises administering to the patient about 70 mg to about 100 mg of the compound of Formula (I) or pharmaceutically acceptable salt thereof per day. In some embodiments, the method comprises administering to the patient about 80 mg to about 200 mg of the compound of Formula (I) or pharmaceutically acceptable salt thereof per day. In some embodiments, the method comprises administering to the patient about 80 mg to about 150 mg of the compound of Formula (I) or pharmaceutically acceptable salt thereof per day. In some embodiments, the method comprises administering to the patient about 80 mg to about 100 mg of the compound of Formula (I) or pharmaceutically acceptable salt thereof per day. In some embodiments, the method comprises administering to the patient about 90 mg to about 200 mg of the compound of Formula (I) or pharmaceutically acceptable salt thereof per day. In some embodiments, the method comprises administering to the patient about 90 mg to about 150 mg of the compound of Formula (I) or pharmaceutically acceptable salt thereof per day. In some embodiments, the method comprises administering to the patient about 90 mg to about 100 mg of the compound of Formula (I) or pharmaceutically acceptable salt thereof per day. In some embodiments, the method comprises administering to the patient about 100 mg to about 200 mg of the compound of Formula (I) or pharmaceutically acceptable salt thereof per day. In some embodiments, the method comprises administering to the patient about 100 mg to about 150 mg of the compound of Formula (I) or pharmaceutically acceptable salt thereof per day.

In some embodiments, the method comprises administering to the patient about 50 mg of the compound of Formula (I) or pharmaceutically acceptable salt thereof per day. In some embodiments, the method comprises administering to the patient about 55 mg of the compound of Formula (I) or pharmaceutically acceptable salt thereof per day. In some embodiments, the method comprises administering to the patient about 60 mg of the compound of Formula (I) or pharmaceutically acceptable salt thereof per day. In some embodiments, the method comprises administering to the patient about 65 mg of the compound of Formula (I) or pharmaceutically acceptable salt thereof per day. In some embodiments, the method comprises administering to the patient about 70 mg of the compound of Formula (I) or pharmaceutically acceptable salt thereof per day. In some embodiments, the method comprises administering to the patient about 75 mg of the compound of Formula (I) or pharmaceutically acceptable salt thereof per day. In some embodiments, the method comprises administering to the patient about 80 mg of the compound of Formula (I) or pharmaceutically acceptable salt thereof per day. In some embodiments, the method comprises administering to the patient about 85 mg of the compound of Formula (I) or pharmaceutically acceptable salt thereof per day. In some embodiments, the method comprises administering to the patient about 90 mg of the compound of Formula (I) or pharmaceutically acceptable salt thereof per day. In some embodiments, the method comprises administering to the patient about 95 mg of the compound of Formula (I) or pharmaceutically acceptable salt thereof per day. In some embodiments, the method comprises administering to the patient about 100 mg of the compound of Formula (I) or pharmaceutically acceptable salt thereof per day. In some embodiments, the method comprises administering to the patient about 105 mg of the compound of Formula (I) or pharmaceutically acceptable salt thereof per day. In some embodiments, the method comprises administering to the patient about 110 mg of the compound of Formula (I) or pharmaceutically acceptable salt thereof per day. In some embodiments, the method comprises administering to the patient about 115 mg of the compound of Formula (I) or pharmaceutically acceptable salt thereof per day. In some embodiments, the method comprises administering to the patient about 120 mg of the compound of Formula (I) or pharmaceutically acceptable salt thereof per day. In some embodiments, the method comprises administering to the patient about 125 mg of the compound of Formula (I) or pharmaceutically acceptable salt thereof per day. In some embodiments, the method comprises administering to the patient about 130 mg of the compound of Formula (I) or pharmaceutically acceptable salt thereof per day. In some embodiments, the method comprises administering to the patient about 135 mg of the compound of Formula (I) or pharmaceutically acceptable salt thereof per day. In some embodiments, the method comprises administering to the patient about 140 mg of the compound of Formula (I) or pharmaceutically acceptable salt thereof per day. In some embodiments, the method comprises administering to the patient about 145 mg of the compound of Formula (I) or pharmaceutically acceptable salt thereof per day. In some embodiments, the method comprises administering to the patient about 150 mg of the compound of Formula (I) or pharmaceutically acceptable salt thereof per day. In some embodiments, the method comprises administering to the patient about 155 mg of the compound of Formula (I) or pharmaceutically acceptable salt thereof per day. In some embodiments, the method comprises administering to the patient about 160 mg of the compound of Formula (I) or pharmaceutically acceptable salt thereof per day. In some embodiments, the method comprises administering to the patient about 165 mg of the compound of Formula (I) or pharmaceutically acceptable salt thereof per day. In some embodiments, the method comprises administering to the patient about 170 mg of the compound of Formula (I) or pharmaceutically acceptable salt thereof per day. In some embodiments, the method comprises administering to the patient about 175 mg of the compound of Formula (I) or pharmaceutically acceptable salt thereof per day. In some embodiments, the method comprises administering to the patient about 180 mg of the compound of Formula (I) or pharmaceutically acceptable salt thereof per day. In some embodiments, the method comprises administering to the patient about 185 mg of the compound of Formula (I) or pharmaceutically acceptable salt thereof per day. In some embodiments, the method comprises administering to the patient about 190 mg of the compound of Formula (I) or pharmaceutically acceptable salt thereof per day. In some embodiments, the method comprises administering to the patient about 195 mg of the compound of Formula (I) or pharmaceutically acceptable salt thereof per day. In some embodiments, the method comprises administering to the patient about 200 mg of the compound of Formula (I) or pharmaceutically acceptable salt thereof per day.

In some embodiments, the method comprises administering to the patient about 50 mg to about 200 mg of the compound of Formula (I) or pharmaceutically acceptable salt thereof once per day. In some embodiments, the method comprises administering to the patient about 50 mg to about 150 mg of the compound of Formula (I) or pharmaceutically acceptable salt thereof once per day. In some embodiments, the method comprises administering to the patient about 50 mg to about 100 mg of the compound of Formula (I) or pharmaceutically acceptable salt thereof once per day. In some embodiments, the method comprises administering to the patient about 60 mg to about 200 mg of the compound of Formula (I) or pharmaceutically acceptable salt thereof once per day. In some embodiments, the method comprises administering to the patient about 60 mg to about 150 mg of the compound of Formula (I) or pharmaceutically acceptable salt thereof once per day. In some embodiments, the method comprises administering to the patient about 60 mg to about 100 mg of the compound of Formula (I) or pharmaceutically acceptable salt thereof once per day. In some embodiments, the method comprises administering to the patient about 70 mg to about 200 mg of the compound of Formula (I) or pharmaceutically acceptable salt thereof once per day. In some embodiments, the method comprises administering to the patient about 70 mg to about 150 mg of the compound of Formula (I) or pharmaceutically acceptable salt thereof once per day. In some embodiments, the method comprises administering to the patient about 70 mg to about 100 mg of the compound of Formula (I) or pharmaceutically acceptable salt thereof once per day. In some embodiments, the method comprises administering to the patient about 80 mg to about 200 mg of the compound of Formula (I) or pharmaceutically acceptable salt thereof once per day. In some embodiments, the method comprises administering to the patient about 80 mg to about 150 mg of the compound of Formula (I) or pharmaceutically acceptable salt thereof once per day. In some embodiments, the method comprises administering to the patient about 80 mg to about 100 mg of the compound of Formula (I) or pharmaceutically acceptable salt thereof once per day. In some embodiments, the method comprises administering to the patient about 90 mg to about 200 mg of the compound of Formula (I) or pharmaceutically acceptable salt thereof once per day. In some embodiments, the method comprises administering to the patient about 90 mg to about 150 mg of the compound of Formula (I) or pharmaceutically acceptable salt thereof once per day. In some embodiments, the method comprises administering to the patient about 90 mg to about 100 mg of the compound of Formula (I) or pharmaceutically acceptable salt thereof once per day. In some embodiments, the method comprises administering to the patient about 100 mg to about 200 mg of the compound of Formula (I) or pharmaceutically acceptable salt thereof once per day. In some embodiments, the method comprises administering to the patient about 100 mg to about 150 mg of the compound of Formula (I) or pharmaceutically acceptable salt thereof once per day.

In some embodiments, the method comprises administering to the patient about 50 mg of the compound of Formula (I) or pharmaceutically acceptable salt thereof once per day. In some embodiments, the method comprises administering to the patient about 55 mg of the compound of Formula (I) or pharmaceutically acceptable salt thereof once per day. In some embodiments, the method comprises administering to the patient about 60 mg of the compound of Formula (I) or pharmaceutically acceptable salt thereof once per day. In some embodiments, the method comprises administering to the patient about 65 mg of the compound of Formula (I) or pharmaceutically acceptable salt thereof once per day. In some embodiments, the method comprises administering to the patient about 70 mg of the compound of Formula (I) or pharmaceutically acceptable salt thereof once per day. In some embodiments, the method comprises administering to the patient about 75 mg of the compound of Formula (I) or pharmaceutically acceptable salt thereof once per day. In some embodiments, the method comprises administering to the patient about 80 mg of the compound of Formula (I) or pharmaceutically acceptable salt thereof once per day. In some embodiments, the method comprises administering to the patient about 85 mg of the compound of Formula (I) or pharmaceutically acceptable salt thereof once per day. In some embodiments, the method comprises administering to the patient about 90 mg of the compound of Formula (I) or pharmaceutically acceptable salt thereof once per day. In some embodiments, the method comprises administering to the patient about 95 mg of the compound of Formula (I) or pharmaceutically acceptable salt thereof once per day. In some embodiments, the method comprises administering to the patient about 100 mg of the compound of Formula (I) or pharmaceutically acceptable salt thereof once per day. In some embodiments, the method comprises administering to the patient about 105 mg of the compound of Formula (I) or pharmaceutically acceptable salt thereof once per day. In some embodiments, the method comprises administering to the patient about 110 mg of the compound of Formula (I) or pharmaceutically acceptable salt thereof once per day. In some embodiments, the method comprises administering to the patient about 115 mg of the compound of Formula (I) or pharmaceutically acceptable salt thereof once per day. In some embodiments, the method comprises administering to the patient about 120 mg of the compound of Formula (I) or pharmaceutically acceptable salt thereof once per day. In some embodiments, the method comprises administering to the patient about 125 mg of the compound of Formula (I) or pharmaceutically acceptable salt thereof once per day. In some embodiments, the method comprises administering to the patient about 130 mg of the compound of Formula (I) or pharmaceutically acceptable salt thereof once per day. In some embodiments, the method comprises administering to the patient about 135 mg of the compound of Formula (I) or pharmaceutically acceptable salt thereof once per day. In some embodiments, the method comprises administering to the patient about 140 mg of the compound of Formula (I) or pharmaceutically acceptable salt thereof once per day. In some embodiments, the method comprises administering to the patient about 145 mg of the compound of Formula (I) or pharmaceutically acceptable salt thereof once per day. In some embodiments, the method comprises administering to the patient about 150 mg of the compound of Formula (I) or pharmaceutically acceptable salt thereof once per day. In some embodiments, the method comprises administering to the patient about 155 mg of the compound of Formula (I) or pharmaceutically acceptable salt thereof once per day. In some embodiments, the method comprises administering to the patient about 160 mg of the compound of Formula (I) or pharmaceutically acceptable salt thereof once per day. In some embodiments, the method comprises administering to the patient about 165 mg of the compound of Formula (I) or pharmaceutically acceptable salt thereof once per day. In some embodiments, the method comprises administering to the patient about 170 mg of the compound of Formula (I) or pharmaceutically acceptable salt thereof once per day. In some embodiments, the method comprises administering to the patient about 175 mg of the compound of Formula (I) or pharmaceutically acceptable salt thereof once per day. In some embodiments, the method comprises administering to the patient about 180 mg of the compound of Formula (I) or pharmaceutically acceptable salt thereof once per day. In some embodiments, the method comprises administering to the patient about 185 mg of the compound of Formula (I) or pharmaceutically acceptable salt thereof once per day. In some embodiments, the method comprises administering to the patient about 190 mg of the compound of Formula (I) or pharmaceutically acceptable salt thereof once per day. In some embodiments, the method comprises administering to the patient about 195 mg of the compound of Formula (I) or pharmaceutically acceptable salt thereof once per day. In some embodiments, the method comprises administering to the patient about 200 mg of the compound of Formula (I) or pharmaceutically acceptable salt thereof once per day.

In some embodiments, the method comprises administering to the patient about 50 mg to about 100 mg of the compound of Formula (I) or pharmaceutically acceptable salt thereof twice per day. In some embodiments, the method comprises administering to the patient about 60 mg to about 100 mg of the compound of Formula (I) or pharmaceutically acceptable salt thereof twice per day. In some embodiments, the method comprises administering to the patient about 70 mg to about 100 mg of the compound of Formula (I) or pharmaceutically acceptable salt thereof twice per day. In some embodiments, the method comprises administering to the patient about 80 mg to about 100 mg of the compound of Formula (I) or pharmaceutically acceptable salt thereof twice per day. In some embodiments, the method comprises administering to the patient about 90 mg to about 100 mg of the compound of Formula (I) or pharmaceutically acceptable salt thereof twice per day.

In some embodiments, the method comprises administering to the patient about 50 mg of the compound of Formula (I) or pharmaceutically acceptable salt thereof twice per day. In some embodiments, the method comprises administering to the patient about 55 mg of the compound of Formula (I) or pharmaceutically acceptable salt thereof twice per day. In some embodiments, the method comprises administering to the patient about 60 mg of the compound of Formula (I) or pharmaceutically acceptable salt thereof twice per day. In some embodiments, the method comprises administering to the patient about 65 mg of the compound of Formula (I) or pharmaceutically acceptable salt thereof twice per day. In some embodiments, the method comprises administering to the patient about 70 mg of the compound of Formula (I) or pharmaceutically acceptable salt thereof twice per day. In some embodiments, the method comprises administering to the patient about 75 mg of the compound of Formula (I) or pharmaceutically acceptable salt thereof twice per day. In some embodiments, the method comprises administering to the patient about 80 mg of the compound of Formula (I) or pharmaceutically acceptable salt thereof twice per day. In some embodiments, the method comprises administering to the patient about 85 mg of the compound of Formula (I) or pharmaceutically acceptable salt thereof twice per day. In some embodiments, the method comprises administering to the patient about 90 mg of the compound of Formula (I) or pharmaceutically acceptable salt thereof twice per day. In some embodiments, the method comprises administering to the patient about 95 mg of the compound of Formula (I) or pharmaceutically acceptable salt thereof twice per day. In some embodiments, the method comprises administering to the patient about 100 mg of the compound of Formula (I) or pharmaceutically acceptable salt thereof twice per day.

The compound of Formula (I) or pharmaceutically acceptable salt thereof may, in another embodiment, be administered to the patient on an intermittent basis in the methods of achieving a stable disease condition in a patient having mesothelioma described herein. In some embodiments, the compound of Formula (I) or pharmaceutically acceptable salt thereof is administered the patient every other day. In some embodiments, the compound of Formula (I) or pharmaceutically acceptable salt thereof is administered the patient every other day. In some embodiments, the compound of Formula (I) or pharmaceutically acceptable salt thereof is administered the patient once per week. In some embodiments, the compound of Formula (I) or pharmaceutically acceptable salt thereof is administered the patient twice per week. In some embodiments, the compound of Formula (I) or pharmaceutically acceptable salt thereof is administered the patient three times per week.

In some embodiments, the method comprises administering to the patient about 50 mg to about 200 mg of the compound of Formula (I) or pharmaceutically acceptable salt thereof once a week. In some embodiments, the method comprises administering to the patient about 50 mg to about 150 mg of the compound of Formula (I) or pharmaceutically acceptable salt thereof once a week. In some embodiments, the method comprises administering to the patient about 50 mg to about 100 mg of the compound of Formula (I) or pharmaceutically acceptable salt thereof once a week. In some embodiments, the method comprises administering to the patient about 60 mg to about 200 mg of the compound of Formula (I) or pharmaceutically acceptable salt thereof once a week. In some embodiments, the method comprises administering to the patient about 60 mg to about 150 mg of the compound of Formula (I) or pharmaceutically acceptable salt thereof once a week. In some embodiments, the method comprises administering to the patient about 60 mg to about 100 mg of the compound of Formula (I) or pharmaceutically acceptable salt thereof once a week. In some embodiments, the method comprises administering to the patient about 70 mg to about 200 mg of the compound of Formula (I) or pharmaceutically acceptable salt thereof once a week. In some embodiments, the method comprises administering to the patient about 70 mg to about 150 mg of the compound of Formula (I) or pharmaceutically acceptable salt thereof once a week. In some embodiments, the method comprises administering to the patient about 70 mg to about 100 mg of the compound of Formula (I) or pharmaceutically acceptable salt thereof once a week. In some embodiments, the method comprises administering to the patient about 80 mg to about 200 mg of the compound of Formula (I) or pharmaceutically acceptable salt thereof once a week. In some embodiments, the method comprises administering to the patient about 80 mg to about 150 mg of the compound of Formula (I) or pharmaceutically acceptable salt thereof once a week. In some embodiments, the method comprises administering to the patient about 80 mg to about 100 mg of the compound of Formula (I) or pharmaceutically acceptable salt thereof once a week. In some embodiments, the method comprises administering to the patient about 90 mg to about 200 mg of the compound of Formula (I) or pharmaceutically acceptable salt thereof once a week. In some embodiments, the method comprises administering to the patient about 90 mg to about 150 mg of the compound of Formula (I) or pharmaceutically acceptable salt thereof once a week. In some embodiments, the method comprises administering to the patient about 90 mg to about 100 mg of the compound of Formula (I) or pharmaceutically acceptable salt thereof once a week. In some embodiments, the method comprises administering to the patient about 100 mg to about 200 mg of the compound of Formula (I) or pharmaceutically acceptable salt thereof once a week. In some embodiments, the method comprises administering to the patient about 100 mg to about 150 mg of the compound of Formula (I) or pharmaceutically acceptable salt thereof once a week.

In some embodiments, the method comprises administering to the patient about 50 mg of the compound of Formula (I) or pharmaceutically acceptable salt thereof once a week. In some embodiments, the method comprises administering to the patient about 55 mg of the compound of Formula (I) or pharmaceutically acceptable salt thereof once a week. In some embodiments, the method comprises administering to the patient about 60 mg of the compound of Formula (I) or pharmaceutically acceptable salt thereof once a week. In some embodiments, the method comprises administering to the patient about 65 mg of the compound of Formula (I) or pharmaceutically acceptable salt thereof once a week. In some embodiments, the method comprises administering to the patient about 70 mg of the compound of Formula (I) or pharmaceutically acceptable salt thereof once a week. In some embodiments, the method comprises administering to the patient about 75 mg of the compound of Formula (I) or pharmaceutically acceptable salt thereof once a week. In some embodiments, the method comprises administering to the patient about 80 mg of the compound of Formula (I) or pharmaceutically acceptable salt thereof once a week. In some embodiments, the method comprises administering to the patient about 85 mg of the compound of Formula (I) or pharmaceutically acceptable salt thereof once a week. In some embodiments, the method comprises administering to the patient about 90 mg of the compound of Formula (I) or pharmaceutically acceptable salt thereof once a week. In some embodiments, the method comprises administering to the patient about 95 mg of the compound of Formula (I) or pharmaceutically acceptable salt thereof once a week. In some embodiments, the method comprises administering to the patient about 100 mg of the compound of Formula (I) or pharmaceutically acceptable salt thereof once a week. In some embodiments, the method comprises administering to the patient about 105 mg of the compound of Formula (I) or pharmaceutically acceptable salt thereof once a week. In some embodiments, the method comprises administering to the patient about 110 mg of the compound of Formula (I) or pharmaceutically acceptable salt thereof once a week. In some embodiments, the method comprises administering to the patient about 115 mg of the compound of Formula (I) or pharmaceutically acceptable salt thereof once a week. In some embodiments, the method comprises administering to the patient about 120 mg of the compound of Formula (I) or pharmaceutically acceptable salt thereof once a week. In some embodiments, the method comprises administering to the patient about 125 mg of the compound of Formula (I) or pharmaceutically acceptable salt thereof once a week. In some embodiments, the method comprises administering to the patient about 130 mg of the compound of Formula (I) or pharmaceutically acceptable salt thereof once a week. In some embodiments, the method comprises administering to the patient about 135 mg of the compound of Formula (I) or pharmaceutically acceptable salt thereof once a week. In some embodiments, the method comprises administering to the patient about 140 mg of the compound of Formula (I) or pharmaceutically acceptable salt thereof once a week. In some embodiments, the method comprises administering to the patient about 145 mg of the compound of Formula (I) or pharmaceutically acceptable salt thereof once a week. In some embodiments, the method comprises administering to the patient about 150 mg of the compound of Formula (I) or pharmaceutically acceptable salt thereof once a week. In some embodiments, the method comprises administering to the patient about 155 mg of the compound of Formula (I) or pharmaceutically acceptable salt thereof once a week. In some embodiments, the method comprises administering to the patient about 160 mg of the compound of Formula (I) or pharmaceutically acceptable salt thereof once a week. In some embodiments, the method comprises administering to the patient about 165 mg of the compound of Formula (I) or pharmaceutically acceptable salt thereof once a week. In some embodiments, the method comprises administering to the patient about 170 mg of the compound of Formula (I) or pharmaceutically acceptable salt thereof once a week. In some embodiments, the method comprises administering to the patient about 175 mg of the compound of Formula (I) or pharmaceutically acceptable salt thereof once a week. In some embodiments, the method comprises administering to the patient about 180 mg of the compound of Formula (I) or pharmaceutically acceptable salt thereof once a week. In some embodiments, the method comprises administering to the patient about 185 mg of the compound of Formula (I) or pharmaceutically acceptable salt thereof once a week. In some embodiments, the method comprises administering to the patient about 190 mg of the compound of Formula (I) or pharmaceutically acceptable salt thereof once a week. In some embodiments, the method comprises administering to the patient about 195 mg of the compound of Formula (I) or pharmaceutically acceptable salt thereof once a week. In some embodiments, the method comprises administering to the patient about 200 mg of the compound of Formula (I) or pharmaceutically acceptable salt thereof once a week.

In some embodiments, the method comprises administering to the patient about 50 mg to about 200 mg of the compound of Formula (I) or pharmaceutically acceptable salt thereof twice a week. In some embodiments, the method comprises administering to the patient about 50 mg to about 150 mg of the compound of Formula (I) or pharmaceutically acceptable salt thereof twice a week. In some embodiments, the method comprises administering to the patient about 50 mg to about 100 mg of the compound of Formula (I) or pharmaceutically acceptable salt thereof twice a week. In some embodiments, the method comprises administering to the patient about 60 mg to about 200 mg of the compound of Formula (I) or pharmaceutically acceptable salt thereof twice a week. In some embodiments, the method comprises administering to the patient about 60 mg to about 150 mg of the compound of Formula (I) or pharmaceutically acceptable salt thereof twice a week. In some embodiments, the method comprises administering to the patient about 60 mg to about 100 mg of the compound of Formula (I) or pharmaceutically acceptable salt thereof twice a week. In some embodiments, the method comprises administering to the patient about 70 mg to about 200 mg of the compound of Formula (I) or pharmaceutically acceptable salt thereof twice a week. In some embodiments, the method comprises administering to the patient about 70 mg to about 150 mg of the compound of Formula (I) or pharmaceutically acceptable salt thereof twice a week. In some embodiments, the method comprises administering to the patient about 70 mg to about 100 mg of the compound of Formula (I) or pharmaceutically acceptable salt thereof twice a week. In some embodiments, the method comprises administering to the patient about 80 mg to about 200 mg of the compound of Formula (I) or pharmaceutically acceptable salt thereof twice a week. In some embodiments, the method comprises administering to the patient about 80 mg to about 150 mg of the compound of Formula (I) or pharmaceutically acceptable salt thereof twice a week. In some embodiments, the method comprises administering to the patient about 80 mg to about 100 mg of the compound of Formula (I) or pharmaceutically acceptable salt thereof twice a week. In some embodiments, the method comprises administering to the patient about 90 mg to about 200 mg of the compound of Formula (I) or pharmaceutically acceptable salt thereof twice a week. In some embodiments, the method comprises administering to the patient about 90 mg to about 150 mg of the compound of Formula (I) or pharmaceutically acceptable salt thereof twice a week. In some embodiments, the method comprises administering to the patient about 90 mg to about 100 mg of the compound of Formula (I) or pharmaceutically acceptable salt thereof twice a week. In some embodiments, the method comprises administering to the patient about 100 mg to about 200 mg of the compound of Formula (I) or pharmaceutically acceptable salt thereof twice a week. In some embodiments, the method comprises administering to the patient about 100 mg to about 150 mg of the compound of Formula (I) or pharmaceutically acceptable salt thereof twice a week.

In some embodiments, the method comprises administering to the patient about 50 mg of the compound of Formula (I) or pharmaceutically acceptable salt thereof twice a week. In some embodiments, the method comprises administering to the patient about 55 mg of the compound of Formula (I) or pharmaceutically acceptable salt thereof twice a week. In some embodiments, the method comprises administering to the patient about 60 mg of the compound of Formula (I) or pharmaceutically acceptable salt thereof twice a week. In some embodiments, the method comprises administering to the patient about 65 mg of the compound of Formula (I) or pharmaceutically acceptable salt thereof twice a week. In some embodiments, the method comprises administering to the patient about 70 mg of the compound of Formula (I) or pharmaceutically acceptable salt thereof twice a week. In some embodiments, the method comprises administering to the patient about 75 mg of the compound of Formula (I) or pharmaceutically acceptable salt thereof twice a week. In some embodiments, the method comprises administering to the patient about 80 mg of the compound of Formula (I) or pharmaceutically acceptable salt thereof twice a week. In some embodiments, the method comprises administering to the patient about 85 mg of the compound of Formula (I) or pharmaceutically acceptable salt thereof twice a week. In some embodiments, the method comprises administering to the patient about 90 mg of the compound of Formula (I) or pharmaceutically acceptable salt thereof twice a week. In some embodiments, the method comprises administering to the patient about 95 mg of the compound of Formula (I) or pharmaceutically acceptable salt thereof twice a week. In some embodiments, the method comprises administering to the patient about 100 mg of the compound of Formula (I) or pharmaceutically acceptable salt thereof twice a week. In some embodiments, the method comprises administering to the patient about 105 mg of the compound of Formula (I) or pharmaceutically acceptable salt thereof twice a week. In some embodiments, the method comprises administering to the patient about 110 mg of the compound of Formula (I) or pharmaceutically acceptable salt thereof twice a week. In some embodiments, the method comprises administering to the patient about 115 mg of the compound of Formula (I) or pharmaceutically acceptable salt thereof twice a week. In some embodiments, the method comprises administering to the patient about 120 mg of the compound of Formula (I) or pharmaceutically acceptable salt thereof twice a week. In some embodiments, the method comprises administering to the patient about 125 mg of the compound of Formula (I) or pharmaceutically acceptable salt thereof twice a week. In some embodiments, the method comprises administering to the patient about 130 mg of the compound of Formula (I) or pharmaceutically acceptable salt thereof twice a week. In some embodiments, the method comprises administering to the patient about 135 mg of the compound of Formula (I) or pharmaceutically acceptable salt thereof twice a week. In some embodiments, the method comprises administering to the patient about 140 mg of the compound of Formula (I) or pharmaceutically acceptable salt thereof twice a week. In some embodiments, the method comprises administering to the patient about 145 mg of the compound of Formula (I) or pharmaceutically acceptable salt thereof twice a week. In some embodiments, the method comprises administering to the patient about 150 mg of the compound of Formula (I) or pharmaceutically acceptable salt thereof twice a week. In some embodiments, the method comprises administering to the patient about 155 mg of the compound of Formula (I) or pharmaceutically acceptable salt thereof twice a week. In some embodiments, the method comprises administering to the patient about 160 mg of the compound of Formula (I) or pharmaceutically acceptable salt thereof twice a week. In some embodiments, the method comprises administering to the patient about 165 mg of the compound of Formula (I) or pharmaceutically acceptable salt thereof twice a week. In some embodiments, the method comprises administering to the patient about 170 mg of the compound of Formula (I) or pharmaceutically acceptable salt thereof twice a week. In some embodiments, the method comprises administering to the patient about 175 mg of the compound of Formula (I) or pharmaceutically acceptable salt thereof twice a week. In some embodiments, the method comprises administering to the patient about 180 mg of the compound of Formula (I) or pharmaceutically acceptable salt thereof twice a week. In some embodiments, the method comprises administering to the patient about 185 mg of the compound of Formula (I) or pharmaceutically acceptable salt thereof twice a week. In some embodiments, the method comprises administering to the patient about 190 mg of the compound of Formula (I) or pharmaceutically acceptable salt thereof twice a week. In some embodiments, the method comprises administering to the patient about 195 mg of the compound of Formula (I) or pharmaceutically acceptable salt thereof twice a week. In some embodiments, the method comprises administering to the patient about 200 mg of the compound of Formula (I) or pharmaceutically acceptable salt thereof twice a week.

In some embodiments, the method comprises administering to the patient about 50 mg to about 200 mg of the compound of Formula (I) or pharmaceutically acceptable salt thereof three times a week. In some embodiments, the method comprises administering to the patient about 50 mg to about 150 mg of the compound of Formula (I) or pharmaceutically acceptable salt thereof three times a week. In some embodiments, the method comprises administering to the patient about 50 mg to about 100 mg of the compound of Formula (I) or pharmaceutically acceptable salt thereof three times a week. In some embodiments, the method comprises administering to the patient about 60 mg to about 200 mg of the compound of Formula (I) or pharmaceutically acceptable salt thereof three times a week. In some embodiments, the method comprises administering to the patient about 60 mg to about 150 mg of the compound of Formula (I) or pharmaceutically acceptable salt thereof three times a week. In some embodiments, the method comprises administering to the patient about 60 mg to about 100 mg of the compound of Formula (I) or pharmaceutically acceptable salt thereof three times a week. In some embodiments, the method comprises administering to the patient about 70 mg to about 200 mg of the compound of Formula (I) or pharmaceutically acceptable salt thereof three times a week. In some embodiments, the method comprises administering to the patient about 70 mg to about 150 mg of the compound of Formula (I) or pharmaceutically acceptable salt thereof three times a week. In some embodiments, the method comprises administering to the patient about 70 mg to about 100 mg of the compound of Formula (I) or pharmaceutically acceptable salt thereof three times a week. In some embodiments, the method comprises administering to the patient about 80 mg to about 200 mg of the compound of Formula (I) or pharmaceutically acceptable salt thereof three times a week. In some embodiments, the method comprises administering to the patient about 80 mg to about 150 mg of the compound of Formula (I) or pharmaceutically acceptable salt thereof three times a week. In some embodiments, the method comprises administering to the patient about 80 mg to about 100 mg of the compound of Formula (I) or pharmaceutically acceptable salt thereof three times a week. In some embodiments, the method comprises administering to the patient about 90 mg to about 200 mg of the compound of Formula (I) or pharmaceutically acceptable salt thereof three times a week. In some embodiments, the method comprises administering to the patient about 90 mg to about 150 mg of the compound of Formula (I) or pharmaceutically acceptable salt thereof three times a week. In some embodiments, the method comprises administering to the patient about 90 mg to about 100 mg of the compound of Formula (I) or pharmaceutically acceptable salt thereof three times a week. In some embodiments, the method comprises administering to the patient about 100 mg to about 200 mg of the compound of Formula (I) or pharmaceutically acceptable salt thereof three times a week. In some embodiments, the method comprises administering to the patient about 100 mg to about 150 mg of the compound of Formula (I) or pharmaceutically acceptable salt thereof three times a week.

In some embodiments, the method comprises administering to the patient about 50 mg of the compound of Formula (I) or pharmaceutically acceptable salt thereof three times a week. In some embodiments, the method comprises administering to the patient about 55 mg of the compound of Formula (I) or pharmaceutically acceptable salt thereof three times a week. In some embodiments, the method comprises administering to the patient about 60 mg of the compound of Formula (I) or pharmaceutically acceptable salt thereof three times a week. In some embodiments, the method comprises administering to the patient about 65 mg of the compound of Formula (I) or pharmaceutically acceptable salt thereof three times a week. In some embodiments, the method comprises administering to the patient about 70 mg of the compound of Formula (I) or pharmaceutically acceptable salt thereof three times a week. In some embodiments, the method comprises administering to the patient about 75 mg of the compound of Formula (I) or pharmaceutically acceptable salt thereof three times a week. In some embodiments, the method comprises administering to the patient about 80 mg of the compound of Formula (I) or pharmaceutically acceptable salt thereof three times a week. In some embodiments, the method comprises administering to the patient about 85 mg of the compound of Formula (I) or pharmaceutically acceptable salt thereof three times a week. In some embodiments, the method comprises administering to the patient about 90 mg of the compound of Formula (I) or pharmaceutically acceptable salt thereof three times a week. In some embodiments, the method comprises administering to the patient about 95 mg of the compound of Formula (I) or pharmaceutically acceptable salt thereof three times a week. In some embodiments, the method comprises administering to the patient about 100 mg of the compound of Formula (I) or pharmaceutically acceptable salt thereof three times a week. In some embodiments, the method comprises administering to the patient about 105 mg of the compound of Formula (I) or pharmaceutically acceptable salt thereof three times a week. In some embodiments, the method comprises administering to the patient about 110 mg of the compound of Formula (I) or pharmaceutically acceptable salt thereof three times a week. In some embodiments, the method comprises administering to the patient about 115 mg of the compound of Formula (I) or pharmaceutically acceptable salt thereof three times a week. In some embodiments, the method comprises administering to the patient about 120 mg of the compound of Formula (I) or pharmaceutically acceptable salt thereof three times a week. In some embodiments, the method comprises administering to the patient about 125 mg of the compound of Formula (I) or pharmaceutically acceptable salt thereof three times a week. In some embodiments, the method comprises administering to the patient about 130 mg of the compound of Formula (I) or pharmaceutically acceptable salt thereof three times a week. In some embodiments, the method comprises administering to the patient about 135 mg of the compound of Formula (I) or pharmaceutically acceptable salt thereof three times a week. In some embodiments, the method comprises administering to the patient about 140 mg of the compound of Formula (I) or pharmaceutically acceptable salt thereof three times a week. In some embodiments, the method comprises administering to the patient about 145 mg of the compound of Formula (I) or pharmaceutically acceptable salt thereof three times a week. In some embodiments, the method comprises administering to the patient about 150 mg of the compound of Formula (I) or pharmaceutically acceptable salt thereof three times a week. In some embodiments, the method comprises administering to the patient about 155 mg of the compound of Formula (I) or pharmaceutically acceptable salt thereof three times a week. In some embodiments, the method comprises administering to the patient about 160 mg of the compound of Formula (I) or pharmaceutically acceptable salt thereof three times a week. In some embodiments, the method comprises administering to the patient about 165 mg of the compound of Formula (I) or pharmaceutically acceptable salt thereof three times a week. In some embodiments, the method comprises administering to the patient about 170 mg of the compound of Formula (I) or pharmaceutically acceptable salt thereof three times a week. In some embodiments, the method comprises administering to the patient about 175 mg of the compound of Formula (I) or pharmaceutically acceptable salt thereof three times a week. In some embodiments, the method comprises administering to the patient about 180 mg of the compound of Formula (I) or pharmaceutically acceptable salt thereof three times a week. In some embodiments, the method comprises administering to the patient about 185 mg of the compound of Formula (I) or pharmaceutically acceptable salt thereof three times a week. In some embodiments, the method comprises administering to the patient about 190 mg of the compound of Formula (I) or pharmaceutically acceptable salt thereof three times a week. In some embodiments, the method comprises administering to the patient about 195 mg of the compound of Formula (I) or pharmaceutically acceptable salt thereof three times a week. In some embodiments, the method comprises administering to the patient about 200 mg of the compound of Formula (I) or pharmaceutically acceptable salt thereof three times a week.

In some embodiments, the method comprises administering to the patient about 50 mg to about 200 mg of the compound of Formula (I) or pharmaceutically acceptable salt thereof every other day. In some embodiments, the method comprises administering to the patient about 50 mg to about 150 mg of the compound of Formula (I) or pharmaceutically acceptable salt thereof every other day. In some embodiments, the method comprises administering to the patient about 50 mg to about 100 mg of the compound of Formula (I) or pharmaceutically acceptable salt thereof every other day. In some embodiments, the method comprises administering to the patient about 60 mg to about 200 mg of the compound of Formula (I) or pharmaceutically acceptable salt thereof every other day. In some embodiments, the method comprises administering to the patient about 60 mg to about 150 mg of the compound of Formula (I) or pharmaceutically acceptable salt thereof every other day. In some embodiments, the method comprises administering to the patient about 60 mg to about 100 mg of the compound of Formula (I) or pharmaceutically acceptable salt thereof every other day. In some embodiments, the method comprises administering to the patient about 70 mg to about 200 mg of the compound of Formula (I) or pharmaceutically acceptable salt thereof every other day. In some embodiments, the method comprises administering to the patient about 70 mg to about 150 mg of the compound of Formula (I) or pharmaceutically acceptable salt thereof every other day. In some embodiments, the method comprises administering to the patient about 70 mg to about 100 mg of the compound of Formula (I) or pharmaceutically acceptable salt thereof every other day. In some embodiments, the method comprises administering to the patient about 80 mg to about 200 mg of the compound of Formula (I) or pharmaceutically acceptable salt thereof every other day. In some embodiments, the method comprises administering to the patient about 80 mg to about 150 mg of the compound of Formula (I) or pharmaceutically acceptable salt thereof every other day. In some embodiments, the method comprises administering to the patient about 80 mg to about 100 mg of the compound of Formula (I) or pharmaceutically acceptable salt thereof every other day. In some embodiments, the method comprises administering to the patient about 90 mg to about 200 mg of the compound of Formula (I) or pharmaceutically acceptable salt thereof every other day. In some embodiments, the method comprises administering to the patient about 90 mg to about 150 mg of the compound of Formula (I) or pharmaceutically acceptable salt thereof every other day. In some embodiments, the method comprises administering to the patient about 90 mg to about 100 mg of the compound of Formula (I) or pharmaceutically acceptable salt thereof every other day. In some embodiments, the method comprises administering to the patient about 100 mg to about 200 mg of the compound of Formula (I) or pharmaceutically acceptable salt thereof every other day. In some embodiments, the method comprises administering to the patient about 100 mg to about 150 mg of the compound of Formula (I) or pharmaceutically acceptable salt thereof every other day.

In some embodiments, the method comprises administering to the patient about 50 mg of the compound of Formula (I) or pharmaceutically acceptable salt thereof every other day. In some embodiments, the method comprises administering to the patient about 55 mg of the compound of Formula (I) or pharmaceutically acceptable salt thereof every other day. In some embodiments, the method comprises administering to the patient about 60 mg of the compound of Formula (I) or pharmaceutically acceptable salt thereof every other day. In some embodiments, the method comprises administering to the patient about 65 mg of the compound of Formula (I) or pharmaceutically acceptable salt thereof every other day. In some embodiments, the method comprises administering to the patient about 70 mg of the compound of Formula (I) or pharmaceutically acceptable salt thereof every other day. In some embodiments, the method comprises administering to the patient about 75 mg of the compound of Formula (I) or pharmaceutically acceptable salt thereof every other day. In some embodiments, the method comprises administering to the patient about 80 mg of the compound of Formula (I) or pharmaceutically acceptable salt thereof every other day. In some embodiments, the method comprises administering to the patient about 85 mg of the compound of Formula (I) or pharmaceutically acceptable salt thereof every other day. In some embodiments, the method comprises administering to the patient about 90 mg of the compound of Formula (I) or pharmaceutically acceptable salt thereof every other day. In some embodiments, the method comprises administering to the patient about 95 mg of the compound of Formula (I) or pharmaceutically acceptable salt thereof every other day. In some embodiments, the method comprises administering to the patient about 100 mg of the compound of Formula (I) or pharmaceutically acceptable salt thereof every other day. In some embodiments, the method comprises administering to the patient about 105 mg of the compound of Formula (I) or pharmaceutically acceptable salt thereof every other day. In some embodiments, the method comprises administering to the patient about 110 mg of the compound embodiments, the method comprises administering to the patient about 115 mg of the compound of Formula (I) or pharmaceutically acceptable salt thereof every other day. In some embodiments, the method comprises administering to the patient about 120 mg of the compound of Formula (I) or pharmaceutically acceptable salt thereof every other day. In some embodiments, the method comprises administering to the patient about 125 mg of the compound of Formula (I) or pharmaceutically acceptable salt thereof every other day. In some embodiments, the method comprises administering to the patient about 130 mg of the compound of Formula (I) or pharmaceutically acceptable salt thereof every other day. In some embodiments, the method comprises administering to the patient about 135 mg of the compound of Formula (I) or pharmaceutically acceptable salt thereof every other day. In some embodiments, the method comprises administering to the patient about 140 mg of the compound of Formula (I) or pharmaceutically acceptable salt thereof every other day. In some embodiments, the method comprises administering to the patient about 145 mg of the compound of Formula (I) or pharmaceutically acceptable salt thereof every other day. In some embodiments, the method comprises administering to the patient about 150 mg of the compound of Formula (I) or pharmaceutically acceptable salt thereof every other day. In some embodiments, the method comprises administering to the patient about 155 mg of the compound of Formula (I) or pharmaceutically acceptable salt thereof every other day. In some embodiments, the method comprises administering to the patient about 160 mg of the compound of Formula (I) or pharmaceutically acceptable salt thereof every other day. In some embodiments, the method comprises administering to the patient about 165 mg of the compound of Formula (I) or pharmaceutically acceptable salt thereof every other day. In some embodiments, the method comprises administering to the patient about 170 mg of the compound of Formula (I) or pharmaceutically acceptable salt thereof every other day. In some embodiments, the method comprises administering to the patient about 175 mg of the compound of Formula (I) or pharmaceutically acceptable salt thereof every other day. In some embodiments, the method comprises administering to the patient about 180 mg of the compound of Formula (I) or pharmaceutically acceptable salt thereof every other day. In some embodiments, the method comprises administering to the patient about 185 mg of the compound of Formula (I) or pharmaceutically acceptable salt thereof every other day. In some embodiments, the method comprises administering to the patient about 190 mg of the compound of Formula (I) or pharmaceutically acceptable salt thereof every other day. In some embodiments, the method comprises administering to the patient about 195 mg of the compound of Formula (I) or pharmaceutically acceptable salt thereof every other day. In some embodiments, the method comprises administering to the patient about 200 mg of the compound of Formula (I) or pharmaceutically acceptable salt thereof every other day.

In some embodiments, the combination agent is intravenously administered to the patient in need thereof. The one or more combination agents administered to a patient in the methods of achieving a stable disease condition in patients having mesothelioma described herein can be dosed to achieve various concentrations of the combination agents in the patient's plasma, which can be quantified by the area under the curve (AUC) for each combination agent administered, e.g., intravenously administered. In some embodiments, the combination agent, (e.g., carboplatin) is intravenously administered to the patient in an amount sufficient to provide an AUC of about 5 to about 7 (e.g., an AUC of about 5 or about 6). In some embodiments, the method comprises reducing each of the amount of the compound of Formula (I) or pharmaceutically acceptable salt thereof and the combination agent administered to the patient after one or more months of administering to the patient in need thereof.

In some embodiments, the paclitaxel is intravenously administered to the patient, e.g., by intravenous infusion. The intravenously administered paclitaxel can be administered to a patient at various dose levels. In some embodiments, the paclitaxel is administered to the patient at about 50 mg/m² to about 150 mg/m². In some embodiments, the paclitaxel is administered to the patient at about 50 mg/m² to about 100 mg/m². In some embodiments, the paclitaxel is administered to the patient at about 60 mg/m² to about 80 mg/m². In some embodiments, the paclitaxel is administered to the patient at about 60 mg/m². In some embodiments, the paclitaxel is administered to the patient at about 80 mg/m². In some embodiments, the paclitaxel is administered to the patient at about 135 mg/m². In some embodiments, the paclitaxel is administered to the patient at about 175 mg/m².

Furthermore, the paclitaxel can be administered to the patient at various frequencies. In some embodiments, the paclitaxel is administered to the patient once per week (e.g., at about 60 mg/m² once per week or about 80 mg/m² once per week). In some embodiments, the paclitaxel is administered to the patient once every three weeks (e.g., at about 135 mg/m² once every three weeks or about 175 mg/m² once every three weeks). In some embodiments, the paclitaxel is administered to the patient by intravenous infusion once per week. In some embodiments, the paclitaxel is administered to the patient by intravenous infusion once per week for three consecutive weeks. In some embodiments, the method comprises administering paclitaxel to the patient by intravenous infusion for more than one cycle, wherein each cycle comprises administering paclitaxel to the patient by intravenous infusion once per week for three consecutive weeks (e.g., once per week for three consecutive weeks at about 60 mg/m² or about 80 mg/m²). In some embodiments, the method comprises not administering paclitaxel to the patient for at least one week between each cycle.

In some embodiments, the paclitaxel is administered to the patient by infusion once every three weeks. In some embodiments, the paclitaxel is administered to the patient for about 60 minutes once per week. In some embodiments, the paclitaxel is administered to the patient for about 3 hours once per week. In some embodiments, the paclitaxel is administered to the patient for about 60 minutes once every three weeks. In some embodiments, the paclitaxel is administered to the patient for about 3 hours once every three weeks. In some embodiments, the paclitaxel is administered to the patient by intravenous infusion for about 60 minutes once per week. In some embodiments, the paclitaxel is administered to the patient by infusion for about 3 hours once per week. In some embodiments, the paclitaxel is administered to the patient by intravenous infusion for about 60 minutes once every three weeks. In some embodiments, the paclitaxel is administered to the patient by infusion for about 3 hours once every three weeks. In some embodiments, the paclitaxel is continuously administered to the patient for about 60 minutes once per week. In some embodiments, the paclitaxel is continuously administered to the patient for about 3 hours once per week. In some embodiments, the paclitaxel is continuously administered to the patient for about 60 minutes once every three weeks. In some embodiments, the paclitaxel is continuously administered to the patient for about 3 hours once every three weeks. In some embodiments, the paclitaxel is continuously administered to the patient by infusion for about 60 minutes once per week. In some embodiments, the paclitaxel is continuously administered to the patient by intravenous infusion for about 3 hours once per week. In some embodiments, the paclitaxel is continuously administered to the patient by infusion for about 60 minutes once every three weeks. In some embodiments, the paclitaxel is continuously administered to the patient by intravenous infusion for about 3 hours once every three weeks.

The method of achieving a stable disease condition in patients having mesothelioma described herein can further comprise reducing each of the amount of the compound of Formula (I) or pharmaceutically acceptable salt thereof and the combination agent administered to the patient after one or more months of administering said compound of Formula (I) or pharmaceutically acceptable salt thereof and said combination agent to the patient in need thereof.

In embodiments of the methods of achieving at least a stable disease condition in a patient having mesothelioma described herein, the disclosure also contemplates administration of the compound of Formula (I) or a pharmaceutically acceptable salt to a patient in need thereof prior to (neo-adjuvant) or after (adjuvant) surgery (e.g., surgical treatment of a solid tumor). In some embodiments, the compound of Formula (I) or a pharmaceutically acceptable salt is administered as an adjuvant. In some embodiments, the compound of Formula (I) or a pharmaceutically acceptable salt is administered as a neo-adjuvant. In some embodiments, the compound of Formula (I) or a pharmaceutically acceptable salt is administered as a neo-adjuvant and an adjuvant. In some embodiments, the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 100 years. In some embodiments, administration of the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 5 years. In some embodiments, administration of the compound of Formula (I) or a pharmaceutically acceptable salt is administered to a patient in need thereof as a neo-adjuvant for a period of 3 months to 6 months, followed by administration of the compound of Formula (I) or a pharmaceutically acceptable salt as an adjuvant for a period from 1 day to 5 years. In some embodiments, no administration of the compound of Formula (I) or a pharmaceutically acceptable salt occurs prior to surgery. In some embodiments, the compound of Formula (I) or a pharmaceutically acceptable salt is administered as an adjuvant for a period from 1 day to 100 years. In some embodiments, the compound of Formula (I) or a pharmaceutically acceptable salt is administered as an adjuvant for a period from 1 day to 5 years.

### Ovarian Cancer

Also provided herein is a method of treating ovarian cancer (for example, a platinum-resistant ovarian cancer) in a patient in need thereof, comprising administering to the patient an effective amount of the compound of Formula (I)

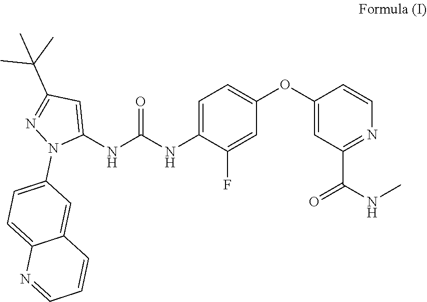 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In an embodiment, the patient has previously been administered at least one prior line of platinum-based therapy.

In some embodiments, the ovarian cancer is characterized as having a serous histology. In some embodiments, the ovarian cancer is characterized as having an endometroid histology. In some embodiments, the ovarian cancer has a mutation selected from the group consisting of a BRCA 1 positive mutation and BRCA 2 positive mutation. In some embodiments, the ovarian cancer does not have a mutation selected from the group consisting of a BRCA 1 positive mutation and BRCA 2 positive mutation. In some embodiments, the cancer is a platinum-resistant ovarian cancer.

A patient treated by the methods of treating an ovarian cancer, such as a platinum-resistant ovarian cancer described herein, can achieve a partial response condition or a stable disease condition. In some embodiments, after 28 days or more of administering the compound of Formula (I) or pharmaceutically acceptable salt thereof to the patient, the patient has at least a partial response or a stable disease condition.

In some embodiments, the ovarian cancer is characterized as having a serous histology. In some embodiments, the ovarian cancer is characterized as having an endometroid histology. In some embodiments, the ovarian cancer has a mutation selected from the group consisting of a BRCA 1 positive mutation and BRCA 2 positive mutation. In some embodiments, the ovarian cancer does not have a mutation selected from the group consisting of a BRCA 1 positive mutation and BRCA 2 positive mutation. In some embodiments, the cancer is a platinum-resistant ovarian cancer.

In embodiments of the methods of treating an ovarian cancer described herein, the compound of Formula (I) or pharmaceutically acceptable salt thereof can be orally administered to the patient in need thereof. In some embodiments, the method comprises administering to the patient about 50 mg to about 200 mg of the compound of Formula (I) or pharmaceutically acceptable salt thereof per day. In certain embodiments, the compound of Formula (I) or pharmaceutically acceptable salt thereof is administered to the patient daily. In some embodiments, the method comprises administering to the patient about 50 mg to about 150 mg of the compound of Formula (I) or pharmaceutically acceptable salt thereof per day. In some embodiments, the method comprises administering to the patient about 50 mg to about 100 mg of the compound of Formula (I) or pharmaceutically acceptable salt thereof per day. In some embodiments, the method comprises administering to the patient about 60 mg to about 200 mg of the compound of Formula (I) or pharmaceutically acceptable salt thereof per day. In some embodiments, the method comprises administering to the patient about 60 mg to about 150 mg of the compound of Formula (I) or pharmaceutically acceptable salt thereof per day. In some embodiments, the method comprises administering to the patient about 60 mg to about 100 mg of the compound of Formula (I) or pharmaceutically acceptable salt thereof per day. In some embodiments, the method comprises administering to the patient about 70 mg to about 200 mg of the compound of Formula (I) or pharmaceutically acceptable salt thereof per day. In some embodiments, the method comprises administering to the patient about 70 mg to about 150 mg of the compound of Formula (I) or pharmaceutically acceptable salt thereof per day. In some embodiments, the method comprises administering to the patient about 70 mg to about 100 mg of the compound of Formula (I) or pharmaceutically acceptable salt thereof per day. In some embodiments, the method comprises administering to the patient about 80 mg to about 200 mg of the compound of Formula (I) or pharmaceutically acceptable salt thereof per day. In some embodiments, the method comprises administering to the patient about 80 mg to about 150 mg of the compound of Formula (I) or pharmaceutically acceptable salt thereof per day. In some embodiments, the method comprises administering to the patient about 80 mg to about 100 mg of the compound of Formula (I) or pharmaceutically acceptable salt thereof per day. In some embodiments, the method comprises administering to the patient about 90 mg to about 200 mg of the compound of Formula (I) or pharmaceutically acceptable salt thereof per day. In some embodiments, the method comprises administering to the patient about 90 mg to about 150 mg of the compound of Formula (I) or pharmaceutically acceptable salt thereof per day. In some embodiments, the method comprises administering to the patient about 90 mg to about 100 mg of the compound of Formula (I) or pharmaceutically acceptable salt thereof per day. In some embodiments, the method comprises administering to the patient about 100 mg to about 200 mg of the compound of Formula (I) or pharmaceutically acceptable salt thereof per day. In some embodiments, the method comprises administering to the patient about 100 mg to about 150 mg of the compound of Formula (I) or pharmaceutically acceptable salt thereof per day.

In some embodiments, the method comprises administering to the patient about 50 mg of the compound of Formula (I) or pharmaceutically acceptable salt thereof per day. In some embodiments, the method comprises administering to the patient about 55 mg of the compound of Formula (I) or pharmaceutically acceptable salt thereof per day. In some embodiments, the method comprises administering to the patient about 60 mg of the compound of Formula (I) or pharmaceutically acceptable salt thereof per day. In some embodiments, the method comprises administering to the patient about 65 mg of the compound of Formula (I) or pharmaceutically acceptable salt thereof per day. In some embodiments, the method comprises administering to the patient about 70 mg of the compound of Formula (I) or pharmaceutically acceptable salt thereof per day. In some embodiments, the method comprises administering to the patient about 75 mg of the compound of Formula (I) or pharmaceutically acceptable salt thereof per day. In some embodiments, the method comprises administering to the patient about 80 mg of the compound of Formula (I) or pharmaceutically acceptable salt thereof per day. In some embodiments, the method comprises administering to the patient about 85 mg of the compound of Formula (I) or pharmaceutically acceptable salt thereof per day. In some embodiments, the method comprises administering to the patient about 90 mg of the compound of Formula (I) or pharmaceutically acceptable salt thereof per day. In some embodiments, the method comprises administering to the patient about 95 mg of the compound of Formula (I) or pharmaceutically acceptable salt thereof per day. In some embodiments, the method comprises administering to the patient about 100 mg of the compound of Formula (I) or pharmaceutically acceptable salt thereof per day. In some embodiments, the method comprises administering to the patient about 105 mg of the compound of Formula (I) or pharmaceutically acceptable salt thereof per day. In some embodiments, the method comprises administering to the patient about 110 mg of the compound of Formula (I) or pharmaceutically acceptable salt thereof per day. In some embodiments, the method comprises administering to the patient about 115 mg of the compound of Formula (I) or pharmaceutically acceptable salt thereof per day. In some embodiments, the method comprises administering to the patient about 120 mg of the compound of Formula (I) or pharmaceutically acceptable salt thereof per day. In some embodiments, the method comprises administering to the patient about 125 mg of the compound of Formula (I) or pharmaceutically acceptable salt thereof per day. In some embodiments, the method comprises administering to the patient about 130 mg of the compound of Formula (I) or pharmaceutically acceptable salt thereof per day. In some embodiments, the method comprises administering to the patient about 135 mg of the compound of Formula (I) or pharmaceutically acceptable salt thereof per day. In some embodiments, the method comprises administering to the patient about 140 mg of the compound of Formula (I) or pharmaceutically acceptable salt thereof per day. In some embodiments, the method comprises administering to the patient about 145 mg of the compound of Formula (I) or pharmaceutically acceptable salt thereof per day. In some embodiments, the method comprises administering to the patient about 150 mg of the compound of Formula (I) or pharmaceutically acceptable salt thereof per day. In some embodiments, the method comprises administering to the patient about 155 mg of the compound of Formula (I) or pharmaceutically acceptable salt thereof per day. In some embodiments, the method comprises administering to the patient about 160 mg of the compound of Formula (I) or pharmaceutically acceptable salt thereof per day. In some embodiments, the method comprises administering to the patient about 165 mg of the compound of Formula (I) or pharmaceutically acceptable salt thereof per day. In some embodiments, the method comprises administering to the patient about 170 mg of the compound of Formula (I) or pharmaceutically acceptable salt thereof per day. In some embodiments, the method comprises administering to the patient about 175 mg of the compound of Formula (I) or pharmaceutically acceptable salt thereof per day. In some embodiments, the method comprises administering to the patient about 180 mg of the compound of Formula (I) or pharmaceutically acceptable salt thereof per day. In some embodiments, the method comprises administering to the patient about 185 mg of the compound of Formula (I) or pharmaceutically acceptable salt thereof per day. In some embodiments, the method comprises administering to the patient about 190 mg of the compound of Formula (I) or pharmaceutically acceptable salt thereof per day. In some embodiments, the method comprises administering to the patient about 195 mg of the compound of Formula (I) or pharmaceutically acceptable salt thereof per day. In some embodiments, the method comprises administering to the patient about 200 mg of the compound of Formula (I) or pharmaceutically acceptable salt thereof per day.

In some embodiments, the method comprises administering to the patient about 50 mg to about 200 mg of the compound of Formula (I) or pharmaceutically acceptable salt thereof once per day. In some embodiments, the method comprises administering to the patient about 50 mg to about 150 mg of the compound of Formula (I) or pharmaceutically acceptable salt thereof once per day. In some embodiments, the method comprises administering to the patient about 50 mg to about 100 mg of the compound of Formula (I) or pharmaceutically acceptable salt thereof once per day. In some embodiments, the method comprises administering to the patient about 60 mg to about 200 mg of the compound of Formula (I) or pharmaceutically acceptable salt thereof once per day. In some embodiments, the method comprises administering to the patient about 60 mg to about 150 mg of the compound of Formula (I) or pharmaceutically acceptable salt thereof once per day. In some embodiments, the method comprises administering to the patient about 60 mg to about 100 mg of the compound of Formula (I) or pharmaceutically acceptable salt thereof once per day. In some embodiments, the method comprises administering to the patient about 70 mg to about 200 mg of the compound of Formula (I) or pharmaceutically acceptable salt thereof once per day. In some embodiments, the method comprises administering to the patient about 70 mg to about 150 mg of the compound of Formula (I) or pharmaceutically acceptable salt thereof once per day. In some embodiments, the method comprises administering to the patient about 70 mg to about 100 mg of the compound of Formula (I) or pharmaceutically acceptable salt thereof once per day. In some embodiments, the method comprises administering to the patient about 80 mg to about 200 mg of the compound of Formula (I) or pharmaceutically acceptable salt thereof once per day. In some embodiments, the method comprises administering to the patient about 80 mg to about 150 mg of the compound of Formula (I) or pharmaceutically acceptable salt thereof once per day. In some embodiments, the method comprises administering to the patient about 80 mg to about 100 mg of the compound of Formula (I) or pharmaceutically acceptable salt thereof once per day. In some embodiments, the method comprises administering to the patient about 90 mg to about 200 mg of the compound of Formula (I) or pharmaceutically acceptable salt thereof once per day. In some embodiments, the method comprises administering to the patient about 90 mg to about 150 mg of the compound of Formula (I) or pharmaceutically acceptable salt thereof once per day. In some embodiments, the method comprises administering to the patient about 90 mg to about 100 mg of the compound of Formula (I) or pharmaceutically acceptable salt thereof once per day. In some embodiments, the method comprises administering to the patient about 100 mg to about 200 mg of the compound of Formula (I) or pharmaceutically acceptable salt thereof once per day. In some embodiments, the method comprises administering to the patient about 100 mg to about 150 mg of the compound of Formula (I) or pharmaceutically acceptable salt thereof once per day.

In some embodiments, the method comprises administering to the patient about 50 mg of the compound of Formula (I) or pharmaceutically acceptable salt thereof once per day. In some embodiments, the method comprises administering to the patient about 55 mg of the compound of Formula (I) or pharmaceutically acceptable salt thereof once per day. In some embodiments, the method comprises administering to the patient about 60 mg of the compound of Formula (I) or pharmaceutically acceptable salt thereof once per day. In some embodiments, the method comprises administering to the patient about 65 mg of the compound of Formula (I) or pharmaceutically acceptable salt thereof once per day. In some embodiments, the method comprises administering to the patient about 70 mg of the compound of Formula (I) or pharmaceutically acceptable salt thereof once per day. In some embodiments, the method comprises administering to the patient about 75 mg of the compound of Formula (I) or pharmaceutically acceptable salt thereof once per day. In some embodiments, the method comprises administering to the patient about 80 mg of the compound of Formula (I) or pharmaceutically acceptable salt thereof once per day. In some embodiments, the method comprises administering to the patient about 85 mg of the compound of Formula (I) or pharmaceutically acceptable salt thereof once per day. In some embodiments, the method comprises administering to the patient about 90 mg of the compound of Formula (I) or pharmaceutically acceptable salt thereof once per day. In some embodiments, the method comprises administering to the patient about 95 mg of the compound of Formula (I) or pharmaceutically acceptable salt thereof once per day. In some embodiments, the method comprises administering to the patient about 100 mg of the compound of Formula (I) or pharmaceutically acceptable salt thereof once per day. In some embodiments, the method comprises administering to the patient about 105 mg of the compound of Formula (I) or pharmaceutically acceptable salt thereof once per day. In some embodiments, the method comprises administering to the patient about 110 mg of the compound of Formula (I) or pharmaceutically acceptable salt thereof once per day. In some embodiments, the method comprises administering to the patient about 115 mg of the compound of Formula (I) or pharmaceutically acceptable salt thereof once per day. In some embodiments, the method comprises administering to the patient about 120 mg of the compound of Formula (I) or pharmaceutically acceptable salt thereof once per day. In some embodiments, the method comprises administering to the patient about 125 mg of the compound of Formula (I) or pharmaceutically acceptable salt thereof once per day. In some embodiments, the method comprises administering to the patient about 130 mg of the compound of Formula (I) or pharmaceutically acceptable salt thereof once per day. In some embodiments, the method comprises administering to the patient about 135 mg of the compound of Formula (I) or pharmaceutically acceptable salt thereof once per day. In some embodiments, the method comprises administering to the patient about 140 mg of the compound of Formula (I) or pharmaceutically acceptable salt thereof once per day. In some embodiments, the method comprises administering to the patient about 145 mg of the compound of Formula (I) or pharmaceutically acceptable salt thereof once per day. In some embodiments, the method comprises administering to the patient about 150 mg of the compound of Formula (I) or pharmaceutically acceptable salt thereof once per day. In some embodiments, the method comprises administering to the patient about 155 mg of the compound of Formula (I) or pharmaceutically acceptable salt thereof once per day. In some embodiments, the method comprises administering to the patient about 160 mg of the compound of Formula (I) or pharmaceutically acceptable salt thereof once per day. In some embodiments, the method comprises administering to the patient about 165 mg of the compound of Formula (I) or pharmaceutically acceptable salt thereof once per day. In some embodiments, the method comprises administering to the patient about 170 mg of the compound of Formula (I) or pharmaceutically acceptable salt thereof once per day. In some embodiments, the method comprises administering to the patient about 175 mg of the compound of Formula (I) or pharmaceutically acceptable salt thereof once per day. In some embodiments, the method comprises administering to the patient about 180 mg of the compound of Formula (I) or pharmaceutically acceptable salt thereof once per day. In some embodiments, the method comprises administering to the patient about 185 mg of the compound of Formula (I) or pharmaceutically acceptable salt thereof once per day. In some embodiments, the method comprises administering to the patient about 190 mg of the compound of Formula (I) or pharmaceutically acceptable salt thereof once per day. In some embodiments, the method comprises administering to the patient about 195 mg of the compound of Formula (I) or pharmaceutically acceptable salt thereof once per day. In some embodiments, the method comprises administering to the patient about 200 mg of the compound of Formula (I) or pharmaceutically acceptable salt thereof once per day.

In some embodiments, the method comprises administering to the patient about 50 mg to about 100 mg of the compound of Formula (I) or pharmaceutically acceptable salt thereof twice per day. In some embodiments, the method comprises administering to the patient about 60 mg to about 100 mg of the compound of Formula (I) or pharmaceutically acceptable salt thereof twice per day. In some embodiments, the method comprises administering to the patient about 70 mg to about 100 mg of the compound of Formula (I) or pharmaceutically acceptable salt thereof twice per day. In some embodiments, the method comprises administering to the patient about 80 mg to about 100 mg of the compound of Formula (I) or pharmaceutically acceptable salt thereof twice per day. In some embodiments, the method comprises administering to the patient about 90 mg to about 100 mg of the compound of Formula (I) or pharmaceutically acceptable salt thereof twice per day.

In some embodiments, the method comprises administering to the patient about 50 mg of the compound of Formula (I) or pharmaceutically acceptable salt thereof twice per day. In some embodiments, the method comprises administering to the patient about 55 mg of the compound of Formula (I) or pharmaceutically acceptable salt thereof twice per day. In some embodiments, the method comprises administering to the patient about 60 mg of the compound of Formula (I) or pharmaceutically acceptable salt thereof twice per day. In some embodiments, the method comprises administering to the patient about 65 mg of the compound of Formula (I) or pharmaceutically acceptable salt thereof twice per day. In some embodiments, the method comprises administering to the patient about 70 mg of the compound of Formula (I) or pharmaceutically acceptable salt thereof twice per day. In some embodiments, the method comprises administering to the patient about 75 mg of the compound of Formula (I) or pharmaceutically acceptable salt thereof twice per day. In some embodiments, the method comprises administering to the patient about 80 mg of the compound of Formula (I) or pharmaceutically acceptable salt thereof twice per day. In some embodiments, the method comprises administering to the patient about 85 mg of the compound of Formula (I) or pharmaceutically acceptable salt thereof twice per day. In some embodiments, the method comprises administering to the patient about 90 mg of the compound of Formula (I) or pharmaceutically acceptable salt thereof twice per day. In some embodiments, the method comprises administering to the patient about 95 mg of the compound of Formula (I) or pharmaceutically acceptable salt thereof twice per day. In some embodiments, the method comprises administering to the patient about 100 mg of the compound of Formula (I) or pharmaceutically acceptable salt thereof twice per day.

The compound of Formula (I) or pharmaceutically acceptable salt thereof may, in another embodiment, be administered to the patient on an intermittent basis in the methods of treating ovarian cancer described herein. In some embodiments, the compound of Formula (I) or pharmaceutically acceptable salt thereof is administered the patient every other day. In some embodiments, the compound of Formula (I) or pharmaceutically acceptable salt thereof is administered the patient every other day. In some embodiments, the compound of Formula (I) or pharmaceutically acceptable salt thereof is administered the patient once per week. In some embodiments, the compound of Formula (I) or pharmaceutically acceptable salt thereof is administered the patient twice per week. In some embodiments, the compound of Formula (I) or pharmaceutically acceptable salt thereof is administered the patient three times per week.

In some embodiments, the method comprises administering to the patient about 50 mg to about 200 mg of the compound of Formula (I) or pharmaceutically acceptable salt thereof once a week. In some embodiments, the method comprises administering to the patient about 50 mg to about 150 mg of the compound of Formula (I) or pharmaceutically acceptable salt thereof once a week. In some embodiments, the method comprises administering to the patient about 50 mg to about 100 mg of the compound of Formula (I) or pharmaceutically acceptable salt thereof once a week. In some embodiments, the method comprises administering to the patient about 60 mg to about 200 mg of the compound of Formula (I) or pharmaceutically acceptable salt thereof once a week. In some embodiments, the method comprises administering to the patient about 60 mg to about 150 mg of the compound of Formula (I) or pharmaceutically acceptable salt thereof once a week. In some embodiments, the method comprises administering to the patient about 60 mg to about 100 mg of the compound of Formula (I) or pharmaceutically acceptable salt thereof once a week. In some embodiments, the method comprises administering to the patient about 70 mg to about 200 mg of the compound of Formula (I) or pharmaceutically acceptable salt thereof once a week. In some embodiments, the method comprises administering to the patient about 70 mg to about 150 mg of the compound of Formula (I) or pharmaceutically acceptable salt thereof once a week. In some embodiments, the method comprises administering to the patient about 70 mg to about 100 mg of the compound of Formula (I) or pharmaceutically acceptable salt thereof once a week. In some embodiments, the method comprises administering to the patient about 80 mg to about 200 mg of the compound of Formula (I) or pharmaceutically acceptable salt thereof once a week. In some embodiments, the method comprises administering to the patient about 80 mg to about 150 mg of the compound of Formula (I) or pharmaceutically acceptable salt thereof once a week. In some embodiments, the method comprises administering to the patient about 80 mg to about 100 mg of the compound of Formula (I) or pharmaceutically acceptable salt thereof once a week. In some embodiments, the method comprises administering to the patient about 90 mg to about 200 mg of the compound of Formula (I) or pharmaceutically acceptable salt thereof once a week. In some embodiments, the method comprises administering to the patient about 90 mg to about 150 mg of the compound of Formula (I) or pharmaceutically acceptable salt thereof once a week. In some embodiments, the method comprises administering to the patient about 90 mg to about 100 mg of the compound of Formula (I) or pharmaceutically acceptable salt thereof once a week. In some embodiments, the method comprises administering to the patient about 100 mg to about 200 mg of the compound of Formula (I) or pharmaceutically acceptable salt thereof once a week. In some embodiments, the method comprises administering to the patient about 100 mg to about 150 mg of the compound of Formula (I) or pharmaceutically acceptable salt thereof once a week.

In some embodiments, the method comprises administering to the patient about 50 mg of the compound of Formula (I) or pharmaceutically acceptable salt thereof once a week. In some embodiments, the method comprises administering to the patient about 55 mg of the compound of Formula (I) or pharmaceutically acceptable salt thereof once a week. In some embodiments, the method comprises administering to the patient about 60 mg of the compound of Formula (I) or pharmaceutically acceptable salt thereof once a week. In some embodiments, the method comprises administering to the patient about 65 mg of the compound of Formula (I) or pharmaceutically acceptable salt thereof once a week. In some embodiments, the method comprises administering to the patient about 70 mg of the compound of Formula (I) or pharmaceutically acceptable salt thereof once a week. In some embodiments, the method comprises administering to the patient about 75 mg of the compound of Formula (I) or pharmaceutically acceptable salt thereof once a week. In some embodiments, the method comprises administering to the patient about 80 mg of the compound of Formula (I) or pharmaceutically acceptable salt thereof once a week. In some embodiments, the method comprises administering to the patient about 85 mg of the compound of Formula (I) or pharmaceutically acceptable salt thereof once a week. In some embodiments, the method comprises administering to the patient about 90 mg of the compound of Formula (I) or pharmaceutically acceptable salt thereof once a week. In some embodiments, the method comprises administering to the patient about 95 mg of the compound of Formula (I) or pharmaceutically acceptable salt thereof once a week. In some embodiments, the method comprises administering to the patient about 100 mg of the compound of Formula (I) or pharmaceutically acceptable salt thereof once a week. In some embodiments, the method comprises administering to the patient about 105 mg of the compound of Formula (I) or pharmaceutically acceptable salt thereof once a week. In some embodiments, the method comprises administering to the patient about 110 mg of the compound of Formula (I) or pharmaceutically acceptable salt thereof once a week. In some embodiments, the method comprises administering to the patient about 115 mg of the compound of Formula (I) or pharmaceutically acceptable salt thereof once a week. In some embodiments, the method comprises administering to the patient about 120 mg of the compound of Formula (I) or pharmaceutically acceptable salt thereof once a week. In some embodiments, the method comprises administering to the patient about 125 mg of the compound of Formula (I) or pharmaceutically acceptable salt thereof once a week. In some embodiments, the method comprises administering to the patient about 130 mg of the compound of Formula (I) or pharmaceutically acceptable salt thereof once a week. In some embodiments, the method comprises administering to the patient about 135 mg of the compound of Formula (I) or pharmaceutically acceptable salt thereof once a week. In some embodiments, the method comprises administering to the patient about 140 mg of the compound of Formula (I) or pharmaceutically acceptable salt thereof once a week. In some embodiments, the method comprises administering to the patient about 145 mg of the compound of Formula (I) or pharmaceutically acceptable salt thereof once a week. In some embodiments, the method comprises administering to the patient about 150 mg of the compound of Formula (I) or pharmaceutically acceptable salt thereof once a week. In some embodiments, the method comprises administering to the patient about 155 mg of the compound of Formula (I) or pharmaceutically acceptable salt thereof once a week. In some embodiments, the method comprises administering to the patient about 160 mg of the compound of Formula (I) or pharmaceutically acceptable salt thereof once a week. In some embodiments, the method comprises administering to the patient about 165 mg of the compound of Formula (I) or pharmaceutically acceptable salt thereof once a week. In some embodiments, the method comprises administering to the patient about 170 mg of the compound of Formula (I) or pharmaceutically acceptable salt thereof once a week. In some embodiments, the method comprises administering to the patient about 175 mg of the compound of Formula (I) or pharmaceutically acceptable salt thereof once a week. In some embodiments, the method comprises administering to the patient about 180 mg of the compound of Formula (I) or pharmaceutically acceptable salt thereof once a week. In some embodiments, the method comprises administering to the patient about 185 mg of the compound of Formula (I) or pharmaceutically acceptable salt thereof once a week. In some embodiments, the method comprises administering to the patient about 190 mg of the compound of Formula (I) or pharmaceutically acceptable salt thereof once a week. In some embodiments, the method comprises administering to the patient about 195 mg of the compound of Formula (I) or pharmaceutically acceptable salt thereof once a week. In some embodiments, the method comprises administering to the patient about 200 mg of the compound of Formula (I) or pharmaceutically acceptable salt thereof once a week.

In some embodiments, the method comprises administering to the patient about 50 mg to about 200 mg of the compound of Formula (I) or pharmaceutically acceptable salt thereof twice a week. In some embodiments, the method comprises administering to the patient about 50 mg to about 150 mg of the compound of Formula (I) or pharmaceutically acceptable salt thereof twice a week. In some embodiments, the method comprises administering to the patient about 50 mg to about 100 mg of the compound of Formula (I) or pharmaceutically acceptable salt thereof twice a week. In some embodiments, the method comprises administering to the patient about 60 mg to about 200 mg of the compound of Formula (I) or pharmaceutically acceptable salt thereof twice a week. In some embodiments, the method comprises administering to the patient about 60 mg to about 150 mg of the compound of Formula (I) or pharmaceutically acceptable salt thereof twice a week. In some embodiments, the method comprises administering to the patient about 60 mg to about 100 mg of the compound of Formula (I) or pharmaceutically acceptable salt thereof twice a week. In some embodiments, the method comprises administering to the patient about 70 mg to about 200 mg of the compound of Formula (I) or pharmaceutically acceptable salt thereof twice a week. In some embodiments, the method comprises administering to the patient about 70 mg to about 150 mg of the compound of Formula (I) or pharmaceutically acceptable salt thereof twice a week. In some embodiments, the method comprises administering to the patient about 70 mg to about 100 mg of the compound of Formula (I) or pharmaceutically acceptable salt thereof twice a week. In some embodiments, the method comprises administering to the patient about 80 mg to about 200 mg of the compound of Formula (I) or pharmaceutically acceptable salt thereof twice a week. In some embodiments, the method comprises administering to the patient about 80 mg to about 150 mg of the compound of Formula (I) or pharmaceutically acceptable salt thereof twice a week. In some embodiments, the method comprises administering to the patient about 80 mg to about 100 mg of the compound of Formula (I) or pharmaceutically acceptable salt thereof twice a week. In some embodiments, the method comprises administering to the patient about 90 mg to about 200 mg of the compound of Formula (I) or pharmaceutically acceptable salt thereof twice a week. In some embodiments, the method comprises administering to the patient about 90 mg to about 150 mg of the compound of Formula (I) or pharmaceutically acceptable salt thereof twice a week. In some embodiments, the method comprises administering to the patient about 90 mg to about 100 mg of the compound of Formula (I) or pharmaceutically acceptable salt thereof twice a week. In some embodiments, the method comprises administering to the patient about 100 mg to about 200 mg of the compound of Formula (I) or pharmaceutically acceptable salt thereof twice a week. In some embodiments, the method comprises administering to the patient about 100 mg to about 150 mg of the compound of Formula (I) or pharmaceutically acceptable salt thereof twice a week.

In some embodiments, the method comprises administering to the patient about 50 mg of the compound of Formula (I) or pharmaceutically acceptable salt thereof twice a week. In some embodiments, the method comprises administering to the patient about 55 mg of the compound of Formula (I) or pharmaceutically acceptable salt thereof twice a week. In some embodiments, the method comprises administering to the patient about 60 mg of the compound of Formula (I) or pharmaceutically acceptable salt thereof twice a week. In some embodiments, the method comprises administering to the patient about 65 mg of the compound of Formula (I) or pharmaceutically acceptable salt thereof twice a week. In some embodiments, the method comprises administering to the patient about 70 mg of the compound of Formula (I) or pharmaceutically acceptable salt thereof twice a week. In some embodiments, the method comprises administering to the patient about 75 mg of the compound of Formula (I) or pharmaceutically acceptable salt thereof twice a week. In some embodiments, the method comprises administering to the patient about 80 mg of the compound of Formula (I) or pharmaceutically acceptable salt thereof twice a week. In some embodiments, the method comprises administering to the patient about 85 mg of the compound of Formula (I) or pharmaceutically acceptable salt thereof twice a week. In some embodiments, the method comprises administering to the patient about 90 mg of the compound of Formula (I) or pharmaceutically acceptable salt thereof twice a week. In some embodiments, the method comprises administering to the patient about 95 mg of the compound of Formula (I) or pharmaceutically acceptable salt thereof twice a week. In some embodiments, the method comprises administering to the patient about 100 mg of the compound of Formula (I) or pharmaceutically acceptable salt thereof twice a week. In some embodiments, the method comprises administering to the patient about 105 mg of the compound of Formula (I) or pharmaceutically acceptable salt thereof twice a week. In some embodiments, the method comprises administering to the patient about 110 mg of the compound of Formula (I) or pharmaceutically acceptable salt thereof twice a week. In some embodiments, the method comprises administering to the patient about 115 mg of the compound of Formula (I) or pharmaceutically acceptable salt thereof twice a week. In some embodiments, the method comprises administering to the patient about 120 mg of the compound of Formula (I) or pharmaceutically acceptable salt thereof twice a week. In some embodiments, the method comprises administering to the patient about 125 mg of the compound of Formula (I) or pharmaceutically acceptable salt thereof twice a week. In some embodiments, the method comprises administering to the patient about 130 mg of the compound of Formula (I) or pharmaceutically acceptable salt thereof twice a week. In some embodiments, the method comprises administering to the patient about 135 mg of the compound of Formula (I) or pharmaceutically acceptable salt thereof twice a week. In some embodiments, the method comprises administering to the patient about 140 mg of the compound of Formula (I) or pharmaceutically acceptable salt thereof twice a week. In some embodiments, the method comprises administering to the patient about 145 mg of the compound of Formula (I) or pharmaceutically acceptable salt thereof twice a week. In some embodiments, the method comprises administering to the patient about 150 mg of the compound of Formula (I) or pharmaceutically acceptable salt thereof twice a week. In some embodiments, the method comprises administering to the patient about 155 mg of the compound of Formula (I) or pharmaceutically acceptable salt thereof twice a week. In some embodiments, the method comprises administering to the patient about 160 mg of the compound of Formula (I) or pharmaceutically acceptable salt thereof twice a week. In some embodiments, the method comprises administering to the patient about 165 mg of the compound of Formula (I) or pharmaceutically acceptable salt thereof twice a week. In some embodiments, the method comprises administering to the patient about 170 mg of the compound of Formula (I) or pharmaceutically acceptable salt thereof twice a week. In some embodiments, the method comprises administering to the patient about 175 mg of the compound of Formula (I) or pharmaceutically acceptable salt thereof twice a week. In some embodiments, the method comprises administering to the patient about 180 mg of the compound of Formula (I) or pharmaceutically acceptable salt thereof twice a week. In some embodiments, the method comprises administering to the patient about 185 mg of the compound of Formula (I) or pharmaceutically acceptable salt thereof twice a week. In some embodiments, the method comprises administering to the patient about 190 mg of the compound of Formula (I) or pharmaceutically acceptable salt thereof twice a week. In some embodiments, the method comprises administering to the patient about 195 mg of the compound of Formula (I) or pharmaceutically acceptable salt thereof twice a week. In some embodiments, the method comprises administering to the patient about 200 mg of the compound of Formula (I) or pharmaceutically acceptable salt thereof twice a week.

In some embodiments, the method comprises administering to the patient about 50 mg to about 200 mg of the compound of Formula (I) or pharmaceutically acceptable salt thereof three times a week. In some embodiments, the method comprises administering to the patient about 50 mg to about 150 mg of the compound of Formula (I) or pharmaceutically acceptable salt thereof three times a week. In some embodiments, the method comprises administering to the patient about 50 mg to about 100 mg of the compound of Formula (I) or pharmaceutically acceptable salt thereof three times a week. In some embodiments, the method comprises administering to the patient about 60 mg to about 200 mg of the compound of Formula (I) or pharmaceutically acceptable salt thereof three times a week. In some embodiments, the method comprises administering to the patient about 60 mg to about 150 mg of the compound of Formula (I) or pharmaceutically acceptable salt thereof three times a week. In some embodiments, the method comprises administering to the patient about 60 mg to about 100 mg of the compound of Formula (I) or pharmaceutically acceptable salt thereof three times a week. In some embodiments, the method comprises administering to the patient about 70 mg to about 200 mg of the compound of Formula (I) or pharmaceutically acceptable salt thereof three times a week. In some embodiments, the method comprises administering to the patient about 70 mg to about 150 mg of the compound of Formula (I) or pharmaceutically acceptable salt thereof three times a week. In some embodiments, the method comprises administering to the patient about 70 mg to about 100 mg of the compound of Formula (I) or pharmaceutically acceptable salt thereof three times a week. In some embodiments, the method comprises administering to the patient about 80 mg to about 200 mg of the compound of Formula (I) or pharmaceutically acceptable salt thereof three times a week. In some embodiments, the method comprises administering to the patient about 80 mg to about 150 mg of the compound of Formula (I) or pharmaceutically acceptable salt thereof three times a week. In some embodiments, the method comprises administering to the patient about 80 mg to about 100 mg of the compound of Formula (I) or pharmaceutically acceptable salt thereof three times a week. In some embodiments, the method comprises administering to the patient about 90 mg to about 200 mg of the compound of Formula (I) or pharmaceutically acceptable salt thereof three times a week. In some embodiments, the method comprises administering to the patient about 90 mg to about 150 mg of the compound of Formula (I) or pharmaceutically acceptable salt thereof three times a week. In some embodiments, the method comprises administering to the patient about 90 mg to about 100 mg of the compound of Formula (I) or pharmaceutically acceptable salt thereof three times a week. In some embodiments, the method comprises administering to the patient about 100 mg to about 200 mg of the compound of Formula (I) or pharmaceutically acceptable salt thereof three times a week. In some embodiments, the method comprises administering to the patient about 100 mg to about 150 mg of the compound of Formula (I) or pharmaceutically acceptable salt thereof three times a week.

In some embodiments, the method comprises administering to the patient about 50 mg of the compound of Formula (I) or pharmaceutically acceptable salt thereof three times a week. In some embodiments, the method comprises administering to the patient about 55 mg of the compound of Formula (I) or pharmaceutically acceptable salt thereof three times a week. In some embodiments, the method comprises administering to the patient about 60 mg of the compound of Formula (I) or pharmaceutically acceptable salt thereof three times a week. In some embodiments, the method comprises administering to the patient about 65 mg of the compound of Formula (I) or pharmaceutically acceptable salt thereof three times a week. In some embodiments, the method comprises administering to the patient about 70 mg of the compound of Formula (I) or pharmaceutically acceptable salt thereof three times a week. In some embodiments, the method comprises administering to the patient about 75 mg of the compound of Formula (I) or pharmaceutically acceptable salt thereof three times a week. In some embodiments, the method comprises administering to the patient about 80 mg of the compound of Formula (I) or pharmaceutically acceptable salt thereof three times a week. In some embodiments, the method comprises administering to the patient about 85 mg of the compound of Formula (I) or pharmaceutically acceptable salt thereof three times a week. In some embodiments, the method comprises administering to the patient about 90 mg of the compound of Formula (I) or pharmaceutically acceptable salt thereof three times a week. In some embodiments, the method comprises administering to the patient about 95 mg of the compound of Formula (I) or pharmaceutically acceptable salt thereof three times a week. In some embodiments, the method comprises administering to the patient about 100 mg of the compound of Formula (I) or pharmaceutically acceptable salt thereof three times a week. In some embodiments, the method comprises administering to the patient about 105 mg of the compound of Formula (I) or pharmaceutically acceptable salt thereof three times a week. In some embodiments, the method comprises administering to the patient about 110 mg of the compound of Formula (I) or pharmaceutically acceptable salt thereof three times a week. In some embodiments, the method comprises administering to the patient about 115 mg of the compound of Formula (I) or pharmaceutically acceptable salt thereof three times a week. In some embodiments, the method comprises administering to the patient about 120 mg of the compound of Formula (I) or pharmaceutically acceptable salt thereof three times a week. In some embodiments, the method comprises administering to the patient about 125 mg of the compound of Formula (I) or pharmaceutically acceptable salt thereof three times a week. In some embodiments, the method comprises administering to the patient about 130 mg of the compound of Formula (I) or pharmaceutically acceptable salt thereof three times a week. In some embodiments, the method comprises administering to the patient about 135 mg of the compound of Formula (I) or pharmaceutically acceptable salt thereof three times a week. In some embodiments, the method comprises administering to the patient about 140 mg of the compound of Formula (I) or pharmaceutically acceptable salt thereof three times a week. In some embodiments, the method comprises administering to the patient about 145 mg of the compound of Formula (I) or pharmaceutically acceptable salt thereof three times a week. In some embodiments, the method comprises administering to the patient about 150 mg of the compound of Formula (I) or pharmaceutically acceptable salt thereof three times a week. In some embodiments, the method comprises administering to the patient about 155 mg of the compound of Formula (I) or pharmaceutically acceptable salt thereof three times a week. In some embodiments, the method comprises administering to the patient about 160 mg of the compound of Formula (I) or pharmaceutically acceptable salt thereof three times a week. In some embodiments, the method comprises administering to the patient about 165 mg of the compound of Formula (I) or pharmaceutically acceptable salt thereof three times a week. In some embodiments, the method comprises administering to the patient about 170 mg of the compound of Formula (I) or pharmaceutically acceptable salt thereof three times a week. In some embodiments, the method comprises administering to the patient about 175 mg of the compound of Formula (I) or pharmaceutically acceptable salt thereof three times a week. In some embodiments, the method comprises administering to the patient about 180 mg of the compound of Formula (I) or pharmaceutically acceptable salt thereof three times a week. In some embodiments, the method comprises administering to the patient about 185 mg of the compound of Formula (I) or pharmaceutically acceptable salt thereof three times a week. In some embodiments, the method comprises administering to the patient about 190 mg of the compound of Formula (I) or pharmaceutically acceptable salt thereof three times a week. In some embodiments, the method comprises administering to the patient about 195 mg of the compound of Formula (I) or pharmaceutically acceptable salt thereof three times a week. In some embodiments, the method comprises administering to the patient about 200 mg of the compound of Formula (I) or pharmaceutically acceptable salt thereof three times a week.

In some embodiments, the method comprises administering to the patient about 50 mg to about 200 mg of the compound of Formula (I) or pharmaceutically acceptable salt thereof every other day. In some embodiments, the method comprises administering to the patient about 50 mg to about 150 mg of the compound of Formula (I) or pharmaceutically acceptable salt thereof every other day. In some embodiments, the method comprises administering to the patient about 50 mg to about 100 mg of the compound of Formula (I) or pharmaceutically acceptable salt thereof every other day. In some embodiments, the method comprises administering to the patient about 60 mg to about 200 mg of the compound of Formula (I) or pharmaceutically acceptable salt thereof every other day. In some embodiments, the method comprises administering to the patient about 60 mg to about 150 mg of the compound of Formula (I) or pharmaceutically acceptable salt thereof every other day. In some embodiments, the method comprises administering to the patient about 60 mg to about 100 mg of the compound of Formula (I) or pharmaceutically acceptable salt thereof every other day. In some embodiments, the method comprises administering to the patient about 70 mg to about 200 mg of the compound of Formula (I) or pharmaceutically acceptable salt thereof every other day. In some embodiments, the method comprises administering to the patient about 70 mg to about 150 mg of the compound of Formula (I) or pharmaceutically acceptable salt thereof every other day. In some embodiments, the method comprises administering to the patient about 70 mg to about 100 mg of the compound of Formula (I) or pharmaceutically acceptable salt thereof every other day. In some embodiments, the method comprises administering to the patient about 80 mg to about 200 mg of the compound of Formula (I) or pharmaceutically acceptable salt thereof every other day. In some embodiments, the method comprises administering to the patient about 80 mg to about 150 mg of the compound of Formula (I) or pharmaceutically acceptable salt thereof every other day. In some embodiments, the method comprises administering to the patient about 80 mg to about 100 mg of the compound of Formula (I) or pharmaceutically acceptable salt thereof every other day. In some embodiments, the method comprises administering to the patient about 90 mg to about 200 mg of the compound of Formula (I) or pharmaceutically acceptable salt thereof every other day. In some embodiments, the method comprises administering to the patient about 90 mg to about 150 mg of the compound of Formula (I) or pharmaceutically acceptable salt thereof every other day. In some embodiments, the method comprises administering to the patient about 90 mg to about 100 mg of the compound of Formula (I) or pharmaceutically acceptable salt thereof every other day. In some embodiments, the method comprises administering to the patient about 100 mg to about 200 mg of the compound of Formula (I) or pharmaceutically acceptable salt thereof every other day. In some embodiments, the method comprises administering to the patient about 100 mg to about 150 mg of the compound of Formula (I) or pharmaceutically acceptable salt thereof every other day.

In some embodiments, the method comprises administering to the patient about 50 mg of the compound of Formula (I) or pharmaceutically acceptable salt thereof every other day. In some embodiments, the method comprises administering to the patient about 55 mg of the compound of Formula (I) or pharmaceutically acceptable salt thereof every other day. In some embodiments, the method comprises administering to the patient about 60 mg of the compound of Formula (I) or pharmaceutically acceptable salt thereof every other day. In some embodiments, the method comprises administering to the patient about 65 mg of the compound of Formula (I) or pharmaceutically acceptable salt thereof every other day. In some embodiments, the method comprises administering to the patient about 70 mg of the compound of Formula (I) or pharmaceutically acceptable salt thereof every other day. In some embodiments, the method comprises administering to the patient about 75 mg of the compound of Formula (I) or pharmaceutically acceptable salt thereof every other day. In some embodiments, the method comprises administering to the patient about 80 mg of the compound of Formula (I) or pharmaceutically acceptable salt thereof every other day. In some embodiments, the method comprises administering to the patient about 85 mg of the compound embodiments, the method comprises administering to the patient about 90 mg of the compound of Formula (I) or pharmaceutically acceptable salt thereof every other day. In some embodiments, the method comprises administering to the patient about 95 mg of the compound of Formula (I) or pharmaceutically acceptable salt thereof every other day. In some embodiments, the method comprises administering to the patient about 100 mg of the compound of Formula (I) or pharmaceutically acceptable salt thereof every other day. In some embodiments, the method comprises administering to the patient about 105 mg of the compound of Formula (I) or pharmaceutically acceptable salt thereof every other day. In some embodiments, the method comprises administering to the patient about 110 mg of the compound of Formula (I) or pharmaceutically acceptable salt thereof every other day. In some embodiments, the method comprises administering to the patient about 115 mg of the compound of Formula (I) or pharmaceutically acceptable salt thereof every other day. In some embodiments, the method comprises administering to the patient about 120 mg of the compound of Formula (I) or pharmaceutically acceptable salt thereof every other day. In some embodiments, the method comprises administering to the patient about 125 mg of the compound of Formula (I) or pharmaceutically acceptable salt thereof every other day. In some embodiments, the method comprises administering to the patient about 130 mg of the compound of Formula (I) or pharmaceutically acceptable salt thereof every other day. In some embodiments, the method comprises administering to the patient about 135 mg of the compound of Formula (I) or pharmaceutically acceptable salt thereof every other day. In some embodiments, the method comprises administering to the patient about 140 mg of the compound of Formula (I) or pharmaceutically acceptable salt thereof every other day. In some embodiments, the method comprises administering to the patient about 145 mg of the compound of Formula (I) or pharmaceutically acceptable salt thereof every other day. In some embodiments, the method comprises administering to the patient about 150 mg of the compound of Formula (I) or pharmaceutically acceptable salt thereof every other day. In some embodiments, the method comprises administering to the patient about 155 mg of the compound of Formula (I) or pharmaceutically acceptable salt thereof every other day. In some embodiments, the method comprises administering to the patient about 160 mg of the compound of Formula (I) or pharmaceutically acceptable salt thereof every other day. In some embodiments, the method comprises administering to the patient about 165 mg of the compound of Formula (I) or pharmaceutically acceptable salt thereof every other day. In some embodiments, the method comprises administering to the patient about 170 mg of the compound of Formula (I) or pharmaceutically acceptable salt thereof every other day. In some embodiments, the method comprises administering to the patient about 175 mg of the compound of Formula (I) or pharmaceutically acceptable salt thereof every other day. In some embodiments, the method comprises administering to the patient about 180 mg of the compound of Formula (I) or pharmaceutically acceptable salt thereof every other day. In some embodiments, the method comprises administering to the patient about 185 mg of the compound of Formula (I) or pharmaceutically acceptable salt thereof every other day. In some embodiments, the method comprises administering to the patient about 190 mg of the compound of Formula (I) or pharmaceutically acceptable salt thereof every other day. In some embodiments, the method comprises administering to the patient about 195 mg of the compound of Formula (I) or pharmaceutically acceptable salt thereof every other day. In some embodiments, the method comprises administering to the patient about 200 mg of the compound of Formula (I) or pharmaceutically acceptable salt thereof every other day.

In some embodiments, a method of treating an ovarian cancer described herein further comprises administering paclitaxel to the patient in need thereof. In one embodiment, the paclitaxel is intravenously administered to the patient, e.g., by intravenous infusion. The intravenously administered paclitaxel can be administered to a patient at various dose levels. In some embodiments, the paclitaxel is administered to the patient at about 50 mg/m² to about 150 mg/m². In some embodiments, the paclitaxel is administered to the patient at about 50 mg/m² to about 100 mg/m². In some embodiments, the paclitaxel is administered to the patient at about 60 mg/m² to about 80 mg/m². In some embodiments, the paclitaxel is administered to the patient at about 60 mg/m². In some embodiments, the paclitaxel is administered to the patient at about 80 mg/m². In some embodiments, the paclitaxel is administered to the patient at about 135 mg/m². In some embodiments, the paclitaxel is administered to the patient at about 175 mg/m².

Furthermore, the paclitaxel can be administered to the patient at various frequencies. In some embodiments, the paclitaxel is administered to the patient once per week (e.g., at about 60 mg/m² once per week or about 80 mg/m² once per week). In some embodiments, the paclitaxel is administered to the patient once every three weeks (e.g., at about 135 mg/m² once every three weeks or about 175 mg/m² once every three weeks). In some embodiments, the paclitaxel is administered to the patient by intravenous infusion once per week. In some embodiments, the paclitaxel is administered to the patient by intravenous infusion once per week for three consecutive weeks. In some embodiments, the method comprises administering paclitaxel to the patient by intravenous infusion for more than one cycle, wherein each cycle comprises administering paclitaxel to the patient by intravenous infusion once per week for three consecutive weeks (e.g., once per week for three consecutive weeks at about 60 mg/m² or about 80 mg/m²). In some embodiments, the method comprises not administering paclitaxel to the patient for at least one week between each cycle.

In some embodiments, the paclitaxel is administered to the patient by infusion once every three weeks. In some embodiments, the paclitaxel is administered to the patient for about 60 minutes once per week. In some embodiments, the paclitaxel is administered to the patient for about 3 hours once per week. In some embodiments, the paclitaxel is administered to the patient for about 60 minutes once every three weeks. In some embodiments, the paclitaxel is administered to the patient for about 3 hours once every three weeks. In some embodiments, the paclitaxel is administered to the patient by intravenous infusion for about 60 minutes once per week. In some embodiments, the paclitaxel is administered to the patient by infusion for about 3 hours once per week. In some embodiments, the paclitaxel is administered to the patient by intravenous infusion for about 60 minutes once every three weeks. In some embodiments, the paclitaxel is administered to the patient by infusion for about 3 hours once every three weeks. In some embodiments, the paclitaxel is continuously administered to the patient for about 60 minutes once per week. In some embodiments, the paclitaxel is continuously administered to the patient for about 3 hours once per week. In some embodiments, the paclitaxel is continuously administered to the patient for about 60 minutes once every three weeks. In some embodiments, the paclitaxel is continuously administered to the patient for about 3 hours once every three weeks. In some embodiments, the paclitaxel is continuously administered to the patient by infusion for about 60 minutes once per week. In some embodiments, the paclitaxel is continuously administered to the patient by intravenous infusion for about 3 hours once per week. In some embodiments, the paclitaxel is continuously administered to the patient by infusion for about 60 minutes once every three weeks. In some embodiments, the paclitaxel is continuously administered to the patient by intravenous infusion for about 3 hours once every three weeks.

In embodiments of the methods of treating an ovarian cancer described herein, the disclosure also contemplates administration of the compound of Formula (I) or a pharmaceutically acceptable salt to a patient in need thereof prior to (neo-adjuvant) or after (adjuvant) surgery (e.g., surgical treatment of a solid tumor). In some embodiments, the compound of Formula (I) or a pharmaceutically acceptable salt is administered as an adjuvant. In some embodiments, the compound of Formula (I) or a pharmaceutically acceptable salt is administered as a neo-adjuvant. In some embodiments, the compound of Formula (I) or a pharmaceutically acceptable salt is administered as a neo-adjuvant and an adjuvant. In some embodiments, the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 100 years. In some embodiments, administration of the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 5 years. In some embodiments, administration of the compound of Formula (I) or a pharmaceutically acceptable salt is administered to a patient in need thereof as a neo-adjuvant for a period of 3 months to 6 months, followed by administration of the compound of Formula (I) or a pharmaceutically acceptable salt as an adjuvant for a period from 1 day to 5 years. In some embodiments, no administration of the compound of Formula (I) or a pharmaceutically acceptable salt occurs prior to surgery. In some embodiments, the compound of Formula (I) or a pharmaceutically acceptable salt is administered as an adjuvant for a period from 1 day to 100 years. In some embodiments, the compound of Formula (I) or a pharmaceutically acceptable salt is administered as an adjuvant for a period from 1 day to 5 years.

In another embodiment, provided herein is a method for achieving at least a partial response in a patient having ovarian cancer, comprising administering to the patient an effective amount of the compound of Formula (I)

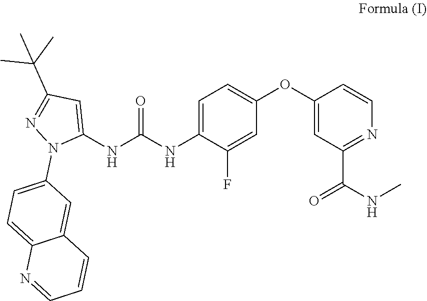 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In an embodiment, the patient has previously been administered at least one prior line of platinum-based therapy. In some embodiments, after 28 days or more of administering the compound of Formula (I) or pharmaceutically acceptable salt thereof to the patient, the patient has at least a partial response or a stable disease condition.

In some embodiments, the ovarian cancer is characterized as having a serous histology. In some embodiments, the ovarian cancer is characterized as having an endometroid histology. In some embodiments, the ovarian cancer has a mutation selected from the group consisting of a BRCA 1 positive mutation and BRCA 2 positive mutation. In some embodiments, the ovarian cancer does not have a mutation selected from the group consisting of a BRCA 1 positive mutation and BRCA 2 positive mutation. In some embodiments, the cancer is a platinum-resistant ovarian cancer.

In embodiments of the methods for achieving at least a partial response in a patient having ovarian cancer described herein, the compound of Formula (I) or pharmaceutically acceptable salt thereof can be orally administered to the patient in need thereof. In certain embodiments, the compound of Formula (I) or pharmaceutically acceptable salt thereof is administered to the patient daily. In some embodiments, the method comprises administering to the patient about 50 mg to about 200 mg of the compound of Formula (I) or pharmaceutically acceptable salt thereof per day. In some embodiments, the method comprises administering to the patient about 50 mg to about 150 mg of the compound of Formula (I) or pharmaceutically acceptable salt thereof per day. In some embodiments, the method comprises administering to the patient about 50 mg to about 100 mg of the compound of Formula (I) or pharmaceutically acceptable salt thereof per day. In some embodiments, the method comprises administering to the patient about 60 mg to about 200 mg of the compound of Formula (I) or pharmaceutically acceptable salt thereof per day. In some embodiments, the method comprises administering to the patient about 60 mg to about 150 mg of the compound of Formula (I) or pharmaceutically acceptable salt thereof per day. In some embodiments, the method comprises administering to the patient about 60 mg to about 100 mg of the compound of Formula (I) or pharmaceutically acceptable salt thereof per day. In some embodiments, the method comprises administering to the patient about 70 mg to about 200 mg of the compound of Formula (I) or pharmaceutically acceptable salt thereof per day. In some embodiments, the method comprises administering to the patient about 70 mg to about 150 mg of the compound of Formula (I) or pharmaceutically acceptable salt thereof per day. In some embodiments, the method comprises administering to the patient about 70 mg to about 100 mg of the compound of Formula (I) or pharmaceutically acceptable salt thereof per day. In some embodiments, the method comprises administering to the patient about 80 mg to about 200 mg of the compound of Formula (I) or pharmaceutically acceptable salt thereof per day. In some embodiments, the method comprises administering to the patient about 80 mg to about 150 mg of the compound of Formula (I) or pharmaceutically acceptable salt thereof per day. In some embodiments, the method comprises administering to the patient about 80 mg to about 100 mg of the compound of Formula (I) or pharmaceutically acceptable salt thereof per day. In some embodiments, the method comprises administering to the patient about 90 mg to about 200 mg of the compound of Formula (I) or pharmaceutically acceptable salt thereof per day. In some embodiments, the method comprises administering to the patient about 90 mg to about 150 mg of the compound of Formula (I) or pharmaceutically acceptable salt thereof per day. In some embodiments, the method comprises administering to the patient about 90 mg to about 100 mg of the compound of Formula (I) or pharmaceutically acceptable salt thereof per day. In some embodiments, the method comprises administering to the patient about 100 mg to about 200 mg of the compound of Formula (I) or pharmaceutically acceptable salt thereof per day. In some embodiments, the method comprises administering to the patient about 100 mg to about 150 mg of the compound of Formula (I) or pharmaceutically acceptable salt thereof per day.

In some embodiments, the method comprises administering to the patient about 50 mg of the compound of Formula (I) or pharmaceutically acceptable salt thereof per day. In some embodiments, the method comprises administering to the patient about 55 mg of the compound of Formula (I) or pharmaceutically acceptable salt thereof per day. In some embodiments, the method comprises administering to the patient about 60 mg of the compound of Formula (I) or pharmaceutically acceptable salt thereof per day. In some embodiments, the method comprises administering to the patient about 65 mg of the compound of Formula (I) or pharmaceutically acceptable salt thereof per day. In some embodiments, the method comprises administering to the patient about 70 mg of the compound of Formula (I) or pharmaceutically acceptable salt thereof per day. In some embodiments, the method comprises administering to the patient about 75 mg of the compound of Formula (I) or pharmaceutically acceptable salt thereof per day. In some embodiments, the method comprises administering to the patient about 80 mg of the compound of Formula (I) or pharmaceutically acceptable salt thereof per day. In some embodiments, the method comprises administering to the patient about 85 mg of the compound of Formula (I) or pharmaceutically acceptable salt thereof per day. In some embodiments, the method comprises administering to the patient about 90 mg of the compound of Formula (I) or pharmaceutically acceptable salt thereof per day. In some embodiments, the method comprises administering to the patient about 95 mg of the compound of Formula (I) or pharmaceutically acceptable salt thereof per day. In some embodiments, the method comprises administering to the patient about 100 mg of the compound of Formula (I) or pharmaceutically acceptable salt thereof per day. In some embodiments, the method comprises administering to the patient about 105 mg of the compound of Formula (I) or pharmaceutically acceptable salt thereof per day. In some embodiments, the method comprises administering to the patient about 110 mg of the compound of Formula (I) or pharmaceutically acceptable salt thereof per day. In some embodiments, the method comprises administering to the patient about 115 mg of the compound of Formula (I) or pharmaceutically acceptable salt thereof per day. In some embodiments, the method comprises administering to the patient about 120 mg of the compound of Formula (I) or pharmaceutically acceptable salt thereof per day. In some embodiments, the method comprises administering to the patient about 125 mg of the compound of Formula (I) or pharmaceutically acceptable salt thereof per day. In some embodiments, the method comprises administering to the patient about 130 mg of the compound of Formula (I) or pharmaceutically acceptable salt thereof per day. In some embodiments, the method comprises administering to the patient about 135 mg of the compound of Formula (I) or pharmaceutically acceptable salt thereof per day. In some embodiments, the method comprises administering to the patient about 140 mg of the compound of Formula (I) or pharmaceutically acceptable salt thereof per day. In some embodiments, the method comprises administering to the patient about 145 mg of the compound of Formula (I) or pharmaceutically acceptable salt thereof per day. In some embodiments, the method comprises administering to the patient about 150 mg of the compound of Formula (I) or pharmaceutically acceptable salt thereof per day. In some embodiments, the method comprises administering to the patient about 155 mg of the compound of Formula (I) or pharmaceutically acceptable salt thereof per day. In some embodiments, the method comprises administering to the patient about 160 mg of the compound of Formula (I) or pharmaceutically acceptable salt thereof per day. In some embodiments, the method comprises administering to the patient about 165 mg of the compound of Formula (I) or pharmaceutically acceptable salt thereof per day. In some embodiments, the method comprises administering to the patient about 170 mg of the compound of Formula (I) or pharmaceutically acceptable salt thereof per day. In some embodiments, the method comprises administering to the patient about 175 mg of the compound of Formula (I) or pharmaceutically acceptable salt thereof per day. In some embodiments, the method comprises administering to the patient about 180 mg of the compound of Formula (I) or pharmaceutically acceptable salt thereof per day. In some embodiments, the method comprises administering to the patient about 185 mg of the compound of Formula (I) or pharmaceutically acceptable salt thereof per day. In some embodiments, the method comprises administering to the patient about 190 mg of the compound of Formula (I) or pharmaceutically acceptable salt thereof per day. In some embodiments, the method comprises administering to the patient about 195 mg of the compound of Formula (I) or pharmaceutically acceptable salt thereof per day. In some embodiments, the method comprises administering to the patient about 200 mg of the compound of Formula (I) or pharmaceutically acceptable salt thereof per day.

In some embodiments, the method comprises administering to the patient about 50 mg to about 200 mg of the compound of Formula (I) or pharmaceutically acceptable salt thereof once per day. In some embodiments, the method comprises administering to the patient about 50 mg to about 150 mg of the compound of Formula (I) or pharmaceutically acceptable salt thereof once per day. In some embodiments, the method comprises administering to the patient about 50 mg to about 100 mg of the compound of Formula (I) or pharmaceutically acceptable salt thereof once per day. In some embodiments, the method comprises administering to the patient about 60 mg to about 200 mg of the compound of Formula (I) or pharmaceutically acceptable salt thereof once per day. In some embodiments, the method comprises administering to the patient about 60 mg to about 150 mg of the compound of Formula (I) or pharmaceutically acceptable salt thereof once per day. In some embodiments, the method comprises administering to the patient about 60 mg to about 100 mg of the compound of Formula (I) or pharmaceutically acceptable salt thereof once per day. In some embodiments, the method comprises administering to the patient about 70 mg to about 200 mg of the compound of Formula (I) or pharmaceutically acceptable salt thereof once per day. In some embodiments, the method comprises administering to the patient about 70 mg to about 150 mg of the compound of Formula (I) or pharmaceutically acceptable salt thereof once per day. In some embodiments, the method comprises administering to the patient about 70 mg to about 100 mg of the compound of Formula (I) or pharmaceutically acceptable salt thereof once per day. In some embodiments, the method comprises administering to the patient about 80 mg to about 200 mg of the compound of Formula (I) or pharmaceutically acceptable salt thereof once per day. In some embodiments, the method comprises administering to the patient about 80 mg to about 150 mg of the compound of Formula (I) or pharmaceutically acceptable salt thereof once per day. In some embodiments, the method comprises administering to the patient about 80 mg to about 100 mg of the compound of Formula (I) or pharmaceutically acceptable salt thereof once per day. In some embodiments, the method comprises administering to the patient about 90 mg to about 200 mg of the compound of Formula (I) or pharmaceutically acceptable salt thereof once per day. In some embodiments, the method comprises administering to the patient about 90 mg to about 150 mg of the compound of Formula (I) or pharmaceutically acceptable salt thereof once per day. In some embodiments, the method comprises administering to the patient about 90 mg to about 100 mg of the compound of Formula (I) or pharmaceutically acceptable salt thereof once per day. In some embodiments, the method comprises administering to the patient about 100 mg to about 200 mg of the compound of Formula (I) or pharmaceutically acceptable salt thereof once per day. In some embodiments, the method comprises administering to the patient about 100 mg to about 150 mg of the compound of Formula (I) or pharmaceutically acceptable salt thereof once per day.

In some embodiments, the method comprises administering to the patient about 50 mg of the compound of Formula (I) or pharmaceutically acceptable salt thereof once per day. In some embodiments, the method comprises administering to the patient about 55 mg of the compound of Formula (I) or pharmaceutically acceptable salt thereof once per day. In some embodiments, the method comprises administering to the patient about 60 mg of the compound of Formula (I) or pharmaceutically acceptable salt thereof once per day. In some embodiments, the method comprises administering to the patient about 65 mg of the compound of Formula (I) or pharmaceutically acceptable salt thereof once per day. In some embodiments, the method comprises administering to the patient about 70 mg of the compound of Formula (I) or pharmaceutically acceptable salt thereof once per day. In some embodiments, the method comprises administering to the patient about 75 mg of the compound of Formula (I) or pharmaceutically acceptable salt thereof once per day. In some embodiments, the method comprises administering to the patient about 80 mg of the compound of Formula (I) or pharmaceutically acceptable salt thereof once per day. In some embodiments, the method comprises administering to the patient about 85 mg of the compound of Formula (I) or pharmaceutically acceptable salt thereof once per day. In some embodiments, the method comprises administering to the patient about 90 mg of the compound of Formula (I) or pharmaceutically acceptable salt thereof once per day. In some embodiments, the method comprises administering to the patient about 95 mg of the compound of Formula (I) or pharmaceutically acceptable salt thereof once per day. In some embodiments, the method comprises administering to the patient about 100 mg of the compound of Formula (I) or pharmaceutically acceptable salt thereof once per day. In some embodiments, the method comprises administering to the patient about 105 mg of the compound of Formula (I) or pharmaceutically acceptable salt thereof once per day. In some embodiments, the method comprises administering to the patient about 110 mg of the compound of Formula (I) or pharmaceutically acceptable salt thereof once per day. In some embodiments, the method comprises administering to the patient about 115 mg of the compound of Formula (I) or pharmaceutically acceptable salt thereof once per day. In some embodiments, the method comprises administering to the patient about 120 mg of the compound of Formula (I) or pharmaceutically acceptable salt thereof once per day. In some embodiments, the method comprises administering to the patient about 125 mg of the compound of Formula (I) or pharmaceutically acceptable salt thereof once per day. In some embodiments, the method comprises administering to the patient about 130 mg of the compound of Formula (I) or pharmaceutically acceptable salt thereof once per day. In some embodiments, the method comprises administering to the patient about 135 mg of the compound of Formula (I) or pharmaceutically acceptable salt thereof once per day. In some embodiments, the method comprises administering to the patient about 140 mg of the compound of Formula (I) or pharmaceutically acceptable salt thereof once per day. In some embodiments, the method comprises administering to the patient about 145 mg of the compound of Formula (I) or pharmaceutically acceptable salt thereof once per day. In some embodiments, the method comprises administering to the patient about 150 mg of the compound of Formula (I) or pharmaceutically acceptable salt thereof once per day. In some embodiments, the method comprises administering to the patient about 155 mg of the compound of Formula (I) or pharmaceutically acceptable salt thereof once per day. In some embodiments, the method comprises administering to the patient about 160 mg of the compound of Formula (I) or pharmaceutically acceptable salt thereof once per day. In some embodiments, the method comprises administering to the patient about 165 mg of the compound of Formula (I) or pharmaceutically acceptable salt thereof once per day. In some embodiments, the method comprises administering to the patient about 170 mg of the compound of Formula (I) or pharmaceutically acceptable salt thereof once per day. In some embodiments, the method comprises administering to the patient about 175 mg of the compound of Formula (I) or pharmaceutically acceptable salt thereof once per day. In some embodiments, the method comprises administering to the patient about 180 mg of the compound of Formula (I) or pharmaceutically acceptable salt thereof once per day. In some embodiments, the method comprises administering to the patient about 185 mg of the compound of Formula (I) or pharmaceutically acceptable salt thereof once per day. In some embodiments, the method comprises administering to the patient about 190 mg of the compound of Formula (I) or pharmaceutically acceptable salt thereof once per day. In some embodiments, the method comprises administering to the patient about 195 mg of the compound of Formula (I) or pharmaceutically acceptable salt thereof once per day. In some embodiments, the method comprises administering to the patient about 200 mg of the compound of Formula (I) or pharmaceutically acceptable salt thereof once per day.

In some embodiments, the method comprises administering to the patient about 50 mg to about 100 mg of the compound of Formula (I) or pharmaceutically acceptable salt thereof twice per day. In some embodiments, the method comprises administering to the patient about 60 mg to about 100 mg of the compound of Formula (I) or pharmaceutically acceptable salt thereof twice per day. In some embodiments, the method comprises administering to the patient about 70 mg to about 100 mg of the compound of Formula (I) or pharmaceutically acceptable salt thereof twice per day. In some embodiments, the method comprises administering to the patient about 80 mg to about 100 mg of the compound of Formula (I) or pharmaceutically acceptable salt thereof twice per day. In some embodiments, the method comprises administering to the patient about 90 mg to about 100 mg of the compound of Formula (I) or pharmaceutically acceptable salt thereof twice per day.

In some embodiments, the method comprises administering to the patient about 50 mg of the compound of Formula (I) or pharmaceutically acceptable salt thereof twice per day. In some embodiments, the method comprises administering to the patient about 55 mg of the compound of Formula (I) or pharmaceutically acceptable salt thereof twice per day. In some embodiments, the method comprises administering to the patient about 60 mg of the compound of Formula (I) or pharmaceutically acceptable salt thereof twice per day. In some embodiments, the method comprises administering to the patient about 65 mg of the compound of Formula (I) or pharmaceutically acceptable salt thereof twice per day. In some embodiments, the method comprises administering to the patient about 70 mg of the compound of Formula (I) or pharmaceutically acceptable salt thereof twice per day. In some embodiments, the method comprises administering to the patient about 75 mg of the compound of Formula (I) or pharmaceutically acceptable salt thereof twice per day. In some embodiments, the method comprises administering to the patient about 80 mg of the compound of Formula (I) or pharmaceutically acceptable salt thereof twice per day. In some embodiments, the method comprises administering to the patient about 85 mg of the compound of Formula (I) or pharmaceutically acceptable salt thereof twice per day. In some embodiments, the method comprises administering to the patient about 90 mg of the compound of Formula (I) or pharmaceutically acceptable salt thereof twice per day. In some embodiments, the method comprises administering to the patient about 95 mg of the compound of Formula (I) or pharmaceutically acceptable salt thereof twice per day. In some embodiments, the method comprises administering to the patient about 100 mg of the compound of Formula (I) or pharmaceutically acceptable salt thereof twice per day.

The compound of Formula (I) or pharmaceutically acceptable salt thereof may, in another embodiment, be administered to the patient on an intermittent basis in the methods for achieving at least a partial response in a patient having ovarian cancer. In some embodiments, the compound of Formula (I) or pharmaceutically acceptable salt thereof is administered the patient every other day. In some embodiments, the compound of Formula (I) or pharmaceutically acceptable salt thereof is administered the patient every other day. In some embodiments, the compound of Formula (I) or pharmaceutically acceptable salt thereof is administered the patient once per week. In some embodiments, the compound of Formula (I) or pharmaceutically acceptable salt thereof is administered the patient twice per week. In some embodiments, the compound of Formula (I) or pharmaceutically acceptable salt thereof is administered the patient three times per week.

In some embodiments, the method comprises administering to the patient about 50 mg to about 200 mg of the compound of Formula (I) or pharmaceutically acceptable salt thereof once a week. In some embodiments, the method comprises administering to the patient about 50 mg to about 150 mg of the compound of Formula (I) or pharmaceutically acceptable salt thereof once a week. In some embodiments, the method comprises administering to the patient about 50 mg to about 100 mg of the compound of Formula (I) or pharmaceutically acceptable salt thereof once a week. In some embodiments, the method comprises administering to the patient about 60 mg to about 200 mg of the compound of Formula (I) or pharmaceutically acceptable salt thereof once a week. In some embodiments, the method comprises administering to the patient about 60 mg to about 150 mg of the compound of Formula (I) or pharmaceutically acceptable salt thereof once a week. In some embodiments, the method comprises administering to the patient about 60 mg to about 100 mg of the compound of Formula (I) or pharmaceutically acceptable salt thereof once a week. In some embodiments, the method comprises administering to the patient about 70 mg to about 200 mg of the compound of Formula (I) or pharmaceutically acceptable salt thereof once a week. In some embodiments, the method comprises administering to the patient about 70 mg to about 150 mg of the compound of Formula (I) or pharmaceutically acceptable salt thereof once a week. In some embodiments, the method comprises administering to the patient about 70 mg to about 100 mg of the compound of Formula (I) or pharmaceutically acceptable salt thereof once a week. In some embodiments, the method comprises administering to the patient about 80 mg to about 200 mg of the compound of Formula (I) or pharmaceutically acceptable salt thereof once a week. In some embodiments, the method comprises administering to the patient about 80 mg to about 150 mg of the compound of Formula (I) or pharmaceutically acceptable salt thereof once a week. In some embodiments, the method comprises administering to the patient about 80 mg to about 100 mg of the compound of Formula (I) or pharmaceutically acceptable salt thereof once a week. In some embodiments, the method comprises administering to the patient about 90 mg to about 200 mg of the compound of Formula (I) or pharmaceutically acceptable salt thereof once a week. In some embodiments, the method comprises administering to the patient about 90 mg to about 150 mg of the compound of Formula (I) or pharmaceutically acceptable salt thereof once a week. In some embodiments, the method comprises administering to the patient about 90 mg to about 100 mg of the compound of Formula (I) or pharmaceutically acceptable salt thereof once a week. In some embodiments, the method comprises administering to the patient about 100 mg to about 200 mg of the compound of Formula (I) or pharmaceutically acceptable salt thereof once a week. In some embodiments, the method comprises administering to the patient about 100 mg to about 150 mg of the compound of Formula (I) or pharmaceutically acceptable salt thereof once a week.

In some embodiments, the method comprises administering to the patient about 50 mg of the compound of Formula (I) or pharmaceutically acceptable salt thereof once a week. In some embodiments, the method comprises administering to the patient about 55 mg of the compound of Formula (I) or pharmaceutically acceptable salt thereof once a week. In some embodiments, the method comprises administering to the patient about 60 mg of the compound of Formula (I) or pharmaceutically acceptable salt thereof once a week. In some embodiments, the method comprises administering to the patient about 65 mg of the compound of Formula (I) or pharmaceutically acceptable salt thereof once a week. In some embodiments, the method comprises administering to the patient about 70 mg of the compound of Formula (I) or pharmaceutically acceptable salt thereof once a week. In some embodiments, the method comprises administering to the patient about 75 mg of the compound of Formula (I) or pharmaceutically acceptable salt thereof once a week. In some embodiments, the method comprises administering to the patient about 80 mg of the compound of Formula (I) or pharmaceutically acceptable salt thereof once a week. In some embodiments, the method comprises administering to the patient about 85 mg of the compound of Formula (I) or pharmaceutically acceptable salt thereof once a week. In some embodiments, the method comprises administering to the patient about 90 mg of the compound of Formula (I) or pharmaceutically acceptable salt thereof once a week. In some embodiments, the method comprises administering to the patient about 95 mg of the compound of Formula (I) or pharmaceutically acceptable salt thereof once a week. In some embodiments, the method comprises administering to the patient about 100 mg of the compound of Formula (I) or pharmaceutically acceptable salt thereof once a week. In some embodiments, the method comprises administering to the patient about 105 mg of the compound of Formula (I) or pharmaceutically acceptable salt thereof once a week. In some embodiments, the method comprises administering to the patient about 110 mg of the compound of Formula (I) or pharmaceutically acceptable salt thereof once a week. In some embodiments, the method comprises administering to the patient about 115 mg of the compound of Formula (I) or pharmaceutically acceptable salt thereof once a week. In some embodiments, the method comprises administering to the patient about 120 mg of the compound of Formula (I) or pharmaceutically acceptable salt thereof once a week. In some embodiments, the method comprises administering to the patient about 125 mg of the compound of Formula (I) or pharmaceutically acceptable salt thereof once a week. In some embodiments, the method comprises administering to the patient about 130 mg of the compound of Formula (I) or pharmaceutically acceptable salt thereof once a week. In some embodiments, the method comprises administering to the patient about 135 mg of the compound of Formula (I) or pharmaceutically acceptable salt thereof once a week. In some embodiments, the method comprises administering to the patient about 140 mg of the compound of Formula (I) or pharmaceutically acceptable salt thereof once a week. In some embodiments, the method comprises administering to the patient about 145 mg of the compound of Formula (I) or pharmaceutically acceptable salt thereof once a week. In some embodiments, the method comprises administering to the patient about 150 mg of the compound of Formula (I) or pharmaceutically acceptable salt thereof once a week. In some embodiments, the method comprises administering to the patient about 155 mg of the compound of Formula (I) or pharmaceutically acceptable salt thereof once a week. In some embodiments, the method comprises administering to the patient about 160 mg of the compound of Formula (I) or pharmaceutically acceptable salt thereof once a week. In some embodiments, the method comprises administering to the patient about 165 mg of the compound of Formula (I) or pharmaceutically acceptable salt thereof once a week. In some embodiments, the method comprises administering to the patient about 170 mg of the compound of Formula (I) or pharmaceutically acceptable salt thereof once a week. In some embodiments, the method comprises administering to the patient about 175 mg of the compound of Formula (I) or pharmaceutically acceptable salt thereof once a week. In some embodiments, the method comprises administering to the patient about 180 mg of the compound of Formula (I) or pharmaceutically acceptable salt thereof once a week. In some embodiments, the method comprises administering to the patient about 185 mg of the compound of Formula (I) or pharmaceutically acceptable salt thereof once a week. In some embodiments, the method comprises administering to the patient about 190 mg of the compound of Formula (I) or pharmaceutically acceptable salt thereof once a week. In some embodiments, the method comprises administering to the patient about 195 mg of the compound of Formula (I) or pharmaceutically acceptable salt thereof once a week. In some embodiments, the method comprises administering to the patient about 200 mg of the compound of Formula (I) or pharmaceutically acceptable salt thereof once a week.

In some embodiments, the method comprises administering to the patient about 50 mg to about 200 mg of the compound of Formula (I) or pharmaceutically acceptable salt thereof twice a week. In some embodiments, the method comprises administering to the patient about 50 mg to about 150 mg of the compound of Formula (I) or pharmaceutically acceptable salt thereof twice a week. In some embodiments, the method comprises administering to the patient about 50 mg to about 100 mg of the compound of Formula (I) or pharmaceutically acceptable salt thereof twice a week. In some embodiments, the method comprises administering to the patient about 60 mg to about 200 mg of the compound of Formula (I) or pharmaceutically acceptable salt thereof twice a week. In some embodiments, the method comprises administering to the patient about 60 mg to about 150 mg of the compound of Formula (I) or pharmaceutically acceptable salt thereof twice a week. In some embodiments, the method comprises administering to the patient about 60 mg to about 100 mg of the compound of Formula (I) or pharmaceutically acceptable salt thereof twice a week. In some embodiments, the method comprises administering to the patient about 70 mg to about 200 mg of the compound of Formula (I) or pharmaceutically acceptable salt thereof twice a week. In some embodiments, the method comprises administering to the patient about 70 mg to about 150 mg of the compound of Formula (I) or pharmaceutically acceptable salt thereof twice a week. In some embodiments, the method comprises administering to the patient about 70 mg to about 100 mg of the compound of Formula (I) or pharmaceutically acceptable salt thereof twice a week. In some embodiments, the method comprises administering to the patient about 80 mg to about 200 mg of the compound of Formula (I) or pharmaceutically acceptable salt thereof twice a week. In some embodiments, the method comprises administering to the patient about 80 mg to about 150 mg of the compound of Formula (I) or pharmaceutically acceptable salt thereof twice a week. In some embodiments, the method comprises administering to the patient about 80 mg to about 100 mg of the compound of Formula (I) or pharmaceutically acceptable salt thereof twice a week. In some embodiments, the method comprises administering to the patient about 90 mg to about 200 mg of the compound of Formula (I) or pharmaceutically acceptable salt thereof twice a week. In some embodiments, the method comprises administering to the patient about 90 mg to about 150 mg of the compound of Formula (I) or pharmaceutically acceptable salt thereof twice a week. In some embodiments, the method comprises administering to the patient about 90 mg to about 100 mg of the compound of Formula (I) or pharmaceutically acceptable salt thereof twice a week. In some embodiments, the method comprises administering to the patient about 100 mg to about 200 mg of the compound of Formula (I) or pharmaceutically acceptable salt thereof twice a week. In some embodiments, the method comprises administering to the patient about 100 mg to about 150 mg of the compound of Formula (I) or pharmaceutically acceptable salt thereof twice a week.

In some embodiments, the method comprises administering to the patient about 50 mg of the compound of Formula (I) or pharmaceutically acceptable salt thereof twice a week. In some embodiments, the method comprises administering to the patient about 55 mg of the compound of Formula (I) or pharmaceutically acceptable salt thereof twice a week. In some embodiments, the method comprises administering to the patient about 60 mg of the compound of Formula (I) or pharmaceutically acceptable salt thereof twice a week. In some embodiments, the method comprises administering to the patient about 65 mg of the compound of Formula (I) or pharmaceutically acceptable salt thereof twice a week. In some embodiments, the method comprises administering to the patient about 70 mg of the compound of Formula (I) or pharmaceutically acceptable salt thereof twice a week. In some embodiments, the method comprises administering to the patient about 75 mg of the compound of Formula (I) or pharmaceutically acceptable salt thereof twice a week. In some embodiments, the method comprises administering to the patient about 80 mg of the compound of Formula (I) or pharmaceutically acceptable salt thereof twice a week. In some embodiments, the method comprises administering to the patient about 85 mg of the compound of Formula (I) or pharmaceutically acceptable salt thereof twice a week. In some embodiments, the method comprises administering to the patient about 90 mg of the compound of Formula (I) or pharmaceutically acceptable salt thereof twice a week. In some embodiments, the method comprises administering to the patient about 95 mg of the compound of Formula (I) or pharmaceutically acceptable salt thereof twice a week. In some embodiments, the method comprises administering to the patient about 100 mg of the compound of Formula (I) or pharmaceutically acceptable salt thereof twice a week. In some embodiments, the method comprises administering to the patient about 105 mg of the compound of Formula (I) or pharmaceutically acceptable salt thereof twice a week. In some embodiments, the method comprises administering to the patient about 110 mg of the compound of Formula (I) or pharmaceutically acceptable salt thereof twice a week. In some embodiments, the method comprises administering to the patient about 115 mg of the compound of Formula (I) or pharmaceutically acceptable salt thereof twice a week. In some embodiments, the method comprises administering to the patient about 120 mg of the compound of Formula (I) or pharmaceutically acceptable salt thereof twice a week. In some embodiments, the method comprises administering to the patient about 125 mg of the compound of Formula (I) or pharmaceutically acceptable salt thereof twice a week. In some embodiments, the method comprises administering to the patient about 130 mg of the compound of Formula (I) or pharmaceutically acceptable salt thereof twice a week. In some embodiments, the method comprises administering to the patient about 135 mg of the compound of Formula (I) or pharmaceutically acceptable salt thereof twice a week. In some embodiments, the method comprises administering to the patient about 140 mg of the compound of Formula (I) or pharmaceutically acceptable salt thereof twice a week. In some embodiments, the method comprises administering to the patient about 145 mg of the compound of Formula (I) or pharmaceutically acceptable salt thereof twice a week. In some embodiments, the method comprises administering to the patient about 150 mg of the compound of Formula (I) or pharmaceutically acceptable salt thereof twice a week. In some embodiments, the method comprises administering to the patient about 155 mg of the compound of Formula (I) or pharmaceutically acceptable salt thereof twice a week. In some embodiments, the method comprises administering to the patient about 160 mg of the compound of Formula (I) or pharmaceutically acceptable salt thereof twice a week. In some embodiments, the method comprises administering to the patient about 165 mg of the compound of Formula (I) or pharmaceutically acceptable salt thereof twice a week. In some embodiments, the method comprises administering to the patient about 170 mg of the compound of Formula (I) or pharmaceutically acceptable salt thereof twice a week. In some embodiments, the method comprises administering to the patient about 175 mg of the compound of Formula (I) or pharmaceutically acceptable salt thereof twice a week. In some embodiments, the method comprises administering to the patient about 180 mg of the compound of Formula (I) or pharmaceutically acceptable salt thereof twice a week. In some embodiments, the method comprises administering to the patient about 185 mg of the compound of Formula (I) or pharmaceutically acceptable salt thereof twice a week. In some embodiments, the method comprises administering to the patient about 190 mg of the compound of Formula (I) or pharmaceutically acceptable salt thereof twice a week. In some embodiments, the method comprises administering to the patient about 195 mg of the compound of Formula (I) or pharmaceutically acceptable salt thereof twice a week. In some embodiments, the method comprises administering to the patient about 200 mg of the compound of Formula (I) or pharmaceutically acceptable salt thereof twice a week.

In some embodiments, the method comprises administering to the patient about 50 mg to about 200 mg of the compound of Formula (I) or pharmaceutically acceptable salt thereof three times a week. In some embodiments, the method comprises administering to the patient about 50 mg to about 150 mg of the compound of Formula (I) or pharmaceutically acceptable salt thereof three times a week. In some embodiments, the method comprises administering to the patient about 50 mg to about 100 mg of the compound of Formula (I) or pharmaceutically acceptable salt thereof three times a week. In some embodiments, the method comprises administering to the patient about 60 mg to about 200 mg of the compound of Formula (I) or pharmaceutically acceptable salt thereof three times a week. In some embodiments, the method comprises administering to the patient about 60 mg to about 150 mg of the compound of Formula (I) or pharmaceutically acceptable salt thereof three times a week. In some embodiments, the method comprises administering to the patient about 60 mg to about 100 mg of the compound of Formula (I) or pharmaceutically acceptable salt thereof three times a week. In some embodiments, the method comprises administering to the patient about 70 mg to about 200 mg of the compound of Formula (I) or pharmaceutically acceptable salt thereof three times a week. In some embodiments, the method comprises administering to the patient about 70 mg to about 150 mg of the compound of Formula (I) or pharmaceutically acceptable salt thereof three times a week. In some embodiments, the method comprises administering to the patient about 70 mg to about 100 mg of the compound of Formula (I) or pharmaceutically acceptable salt thereof three times a week. In some embodiments, the method comprises administering to the patient about 80 mg to about 200 mg of the compound of Formula (I) or pharmaceutically acceptable salt thereof three times a week. In some embodiments, the method comprises administering to the patient about 80 mg to about 150 mg of the compound of Formula (I) or pharmaceutically acceptable salt thereof three times a week. In some embodiments, the method comprises administering to the patient about 80 mg to about 100 mg of the compound of Formula (I) or pharmaceutically acceptable salt thereof three times a week. In some embodiments, the method comprises administering to the patient about 90 mg to about 200 mg of the compound of Formula (I) or pharmaceutically acceptable salt thereof three times a week. In some embodiments, the method comprises administering to the patient about 90 mg to about 150 mg of the compound of Formula (I) or pharmaceutically acceptable salt thereof three times a week. In some embodiments, the method comprises administering to the patient about 90 mg to about 100 mg of the compound of Formula (I) or pharmaceutically acceptable salt thereof three times a week. In some embodiments, the method comprises administering to the patient about 100 mg to about 200 mg of the compound of Formula (I) or pharmaceutically acceptable salt thereof three times a week. In some embodiments, the method comprises administering to the patient about 100 mg to about 150 mg of the compound of Formula (I) or pharmaceutically acceptable salt thereof three times a week.

In some embodiments, the method comprises administering to the patient about 50 mg of the compound of Formula (I) or pharmaceutically acceptable salt thereof three times a week. In some embodiments, the method comprises administering to the patient about 55 mg of the compound of Formula (I) or pharmaceutically acceptable salt thereof three times a week. In some embodiments, the method comprises administering to the patient about 60 mg of the compound of Formula (I) or pharmaceutically acceptable salt thereof three times a week. In some embodiments, the method comprises administering to the patient about 65 mg of the compound of Formula (I) or pharmaceutically acceptable salt thereof three times a week. In some embodiments, the method comprises administering to the patient about 70 mg of the compound of Formula (I) or pharmaceutically acceptable salt thereof three times a week. In some embodiments, the method comprises administering to the patient about 75 mg of the compound of Formula (I) or pharmaceutically acceptable salt thereof three times a week. In some embodiments, the method comprises administering to the patient about 80 mg of the compound of Formula (I) or pharmaceutically acceptable salt thereof three times a week. In some embodiments, the method comprises administering to the patient about 85 mg of the compound of Formula (I) or pharmaceutically acceptable salt thereof three times a week. In some embodiments, the method comprises administering to the patient about 90 mg of the compound of Formula (I) or pharmaceutically acceptable salt thereof three times a week. In some embodiments, the method comprises administering to the patient about 95 mg of the compound of Formula (I) or pharmaceutically acceptable salt thereof three times a week. In some embodiments, the method comprises administering to the patient about 100 mg of the compound of Formula (I) or pharmaceutically acceptable salt thereof three times a week. In some embodiments, the method comprises administering to the patient about 105 mg of the compound of Formula (I) or pharmaceutically acceptable salt thereof three times a week. In some embodiments, the method comprises administering to the patient about 110 mg of the compound of Formula (I) or pharmaceutically acceptable salt thereof three times a week. In some embodiments, the method comprises administering to the patient about 115 mg of the compound of Formula (I) or pharmaceutically acceptable salt thereof three times a week. In some embodiments, the method comprises administering to the patient about 120 mg of the compound of Formula (I) or pharmaceutically acceptable salt thereof three times a week. In some embodiments, the method comprises administering to the patient about 125 mg of the compound of Formula (I) or pharmaceutically acceptable salt thereof three times a week. In some embodiments, the method comprises administering to the patient about 130 mg of the compound of Formula (I) or pharmaceutically acceptable salt thereof three times a week. In some embodiments, the method comprises administering to the patient about 135 mg of the compound of Formula (I) or pharmaceutically acceptable salt thereof three times a week. In some embodiments, the method comprises administering to the patient about 140 mg of the compound of Formula (I) or pharmaceutically acceptable salt thereof three times a week. In some embodiments, the method comprises administering to the patient about 145 mg of the compound of Formula (I) or pharmaceutically acceptable salt thereof three times a week. In some embodiments, the method comprises administering to the patient about 150 mg of the compound of Formula (I) or pharmaceutically acceptable salt thereof three times a week. In some embodiments, the method comprises administering to the patient about 155 mg of the compound of Formula (I) or pharmaceutically acceptable salt thereof three times a week. In some embodiments, the method comprises administering to the patient about 160 mg of the compound of Formula (I) or pharmaceutically acceptable salt thereof three times a week. In some embodiments, the method comprises administering to the patient about 165 mg of the compound of Formula (I) or pharmaceutically acceptable salt thereof three times a week. In some embodiments, the method comprises administering to the patient about 170 mg of the compound of Formula (I) or pharmaceutically acceptable salt thereof three times a week. In some embodiments, the method comprises administering to the patient about 175 mg of the compound of Formula (I) or pharmaceutically acceptable salt thereof three times a week. In some embodiments, the method comprises administering to the patient about 180 mg of the compound of Formula (I) or pharmaceutically acceptable salt thereof three times a week. In some embodiments, the method comprises administering to the patient about 185 mg of the compound of Formula (I) or pharmaceutically acceptable salt thereof three times a week. In some embodiments, the method comprises administering to the patient about 190 mg of the compound of Formula (I) or pharmaceutically acceptable salt thereof three times a week. In some embodiments, the method comprises administering to the patient about 195 mg of the compound of Formula (I) or pharmaceutically acceptable salt thereof three times a week. In some embodiments, the method comprises administering to the patient about 200 mg of the compound of Formula (I) or pharmaceutically acceptable salt thereof three times a week.

In some embodiments, the method comprises administering to the patient about 50 mg to about 200 mg of the compound of Formula (I) or pharmaceutically acceptable salt thereof every other day. In some embodiments, the method comprises administering to the patient about 50 mg to about 150 mg of the compound of Formula (I) or pharmaceutically acceptable salt thereof every other day. In some embodiments, the method comprises administering to the patient about 50 mg to about 100 mg of the compound of Formula (I) or pharmaceutically acceptable salt thereof every other day. In some embodiments, the method comprises administering to the patient about 60 mg to about 200 mg of the compound of Formula (I) or pharmaceutically acceptable salt thereof every other day. In some embodiments, the method comprises administering to the patient about 60 mg to about 150 mg of the compound of Formula (I) or pharmaceutically acceptable salt thereof every other day. In some embodiments, the method comprises administering to the patient about 60 mg to about 100 mg of the compound of Formula (I) or pharmaceutically acceptable salt thereof every other day. In some embodiments, the method comprises administering to the patient about 70 mg to about 200 mg of the compound of Formula (I) or pharmaceutically acceptable salt thereof every other day. In some embodiments, the method comprises administering to the patient about 70 mg to about 150 mg of the compound of Formula (I) or pharmaceutically acceptable salt thereof every other day. In some embodiments, the method comprises administering to the patient about 70 mg to about 100 mg of the compound of Formula (I) or pharmaceutically acceptable salt thereof every other day. In some embodiments, the method comprises administering to the patient about 80 mg to about 200 mg of the compound of Formula (I) or pharmaceutically acceptable salt thereof every other day. In some embodiments, the method comprises administering to the patient about 80 mg to about 150 mg of the compound of Formula (I) or pharmaceutically acceptable salt thereof every other day. In some embodiments, the method comprises administering to the patient about 80 mg to about 100 mg of the compound of Formula (I) or pharmaceutically acceptable salt thereof every other day. In some embodiments, the method comprises administering to the patient about 90 mg to about 200 mg of the compound of Formula (I) or pharmaceutically acceptable salt thereof every other day. In some embodiments, the method comprises administering to the patient about 90 mg to about 150 mg of the compound of Formula (I) or pharmaceutically acceptable salt thereof every other day. In some embodiments, the method comprises administering to the patient about 90 mg to about 100 mg of the compound of Formula (I) or pharmaceutically acceptable salt thereof every other day. In some embodiments, the method comprises administering to the patient about 100 mg to about 200 mg of the compound of Formula (I) or pharmaceutically acceptable salt thereof every other day. In some embodiments, the method comprises administering to the patient about 100 mg to about 150 mg of the compound of Formula (I) or pharmaceutically acceptable salt thereof every other day.

In some embodiments, the method comprises administering to the patient about 50 mg of the compound of Formula (I) or pharmaceutically acceptable salt thereof every other day. In some embodiments, the method comprises administering to the patient about 55 mg of the compound of Formula (I) or pharmaceutically acceptable salt thereof every other day. In some embodiments, the method comprises administering to the patient about 60 mg of the compound of Formula (I) or pharmaceutically acceptable salt thereof every other day. In some embodiments, the method comprises administering to the patient about 65 mg of the compound of Formula (I) or pharmaceutically acceptable salt thereof every other day. In some embodiments, the method comprises administering to the patient about 70 mg of the compound of Formula (I) or pharmaceutically acceptable salt thereof every other day. In some embodiments, the method comprises administering to the patient about 75 mg of the compound of Formula (I) or pharmaceutically acceptable salt thereof every other day. In some embodiments, the method comprises administering to the patient about 80 mg of the compound of Formula (I) or pharmaceutically acceptable salt thereof every other day. In some embodiments, the method comprises administering to the patient about 85 mg of the compound of Formula (I) or pharmaceutically acceptable salt thereof every other day. In some embodiments, the method comprises administering to the patient about 90 mg of the compound of Formula (I) or pharmaceutically acceptable salt thereof every other day. In some embodiments, the method comprises administering to the patient about 95 mg of the compound of Formula (I) or pharmaceutically acceptable salt thereof every other day. In some embodiments, the method comprises administering to the patient about 100 mg of the compound of Formula (I) or pharmaceutically acceptable salt thereof every other day. In some embodiments, the method comprises administering to the patient about 105 mg of the compound of Formula (I) or pharmaceutically acceptable salt thereof every other day. In some embodiments, the method comprises administering to the patient about 110 mg of the compound of Formula (I) or pharmaceutically acceptable salt thereof every other day. In some embodiments, the method comprises administering to the patient about 115 mg of the compound of Formula (I) or pharmaceutically acceptable salt thereof every other day. In some embodiments, the method comprises administering to the patient about 120 mg of the compound of Formula (I) or pharmaceutically acceptable salt thereof every other day. In some embodiments, the method comprises administering to the patient about 125 mg of the compound of Formula (I) or pharmaceutically acceptable salt thereof every other day. In some embodiments, the method comprises administering to the patient about 130 mg of the compound embodiments, the method comprises administering to the patient about 135 mg of the compound of Formula (I) or pharmaceutically acceptable salt thereof every other day. In some embodiments, the method comprises administering to the patient about 140 mg of the compound of Formula (I) or pharmaceutically acceptable salt thereof every other day. In some embodiments, the method comprises administering to the patient about 145 mg of the compound of Formula (I) or pharmaceutically acceptable salt thereof every other day. In some embodiments, the method comprises administering to the patient about 150 mg of the compound of Formula (I) or pharmaceutically acceptable salt thereof every other day. In some embodiments, the method comprises administering to the patient about 155 mg of the compound of Formula (I) or pharmaceutically acceptable salt thereof every other day. In some embodiments, the method comprises administering to the patient about 160 mg of the compound of Formula (I) or pharmaceutically acceptable salt thereof every other day. In some embodiments, the method comprises administering to the patient about 165 mg of the compound of Formula (I) or pharmaceutically acceptable salt thereof every other day. In some embodiments, the method comprises administering to the patient about 170 mg of the compound of Formula (I) or pharmaceutically acceptable salt thereof every other day. In some embodiments, the method comprises administering to the patient about 175 mg of the compound of Formula (I) or pharmaceutically acceptable salt thereof every other day. In some embodiments, the method comprises administering to the patient about 180 mg of the compound of Formula (I) or pharmaceutically acceptable salt thereof every other day. In some embodiments, the method comprises administering to the patient about 185 mg of the compound of Formula (I) or pharmaceutically acceptable salt thereof every other day. In some embodiments, the method comprises administering to the patient about 190 mg of the compound of Formula (I) or pharmaceutically acceptable salt thereof every other day. In some embodiments, the method comprises administering to the patient about 195 mg of the compound of Formula (I) or pharmaceutically acceptable salt thereof every other day. In some embodiments, the method comprises administering to the patient about 200 mg of the compound of Formula (I) or pharmaceutically acceptable salt thereof every other day.

In some embodiments, a method of achieving at least a partial response in a patient having an ovarian cancer described herein further comprises administering paclitaxel to the patient in need thereof. In some embodiments, the paclitaxel is intravenously administered to the patient, e.g., by intravenous infusion. The intravenously administered paclitaxel can be administered to a patient at various dose levels. In some embodiments, the paclitaxel is administered to the patient at about 50 mg/m² to about 150 mg/m². In some embodiments, the paclitaxel is administered to the patient at about 50 mg/m² to about 100 mg/m². In some embodiments, the paclitaxel is administered to the patient at about 60 mg/m² to about 80 mg/m². In some embodiments, the paclitaxel is administered to the patient at about 60 mg/m². In some embodiments, the paclitaxel is administered to the patient at about 80 mg/m². In some embodiments, the paclitaxel is administered to the patient at about 135 mg/m². In some embodiments, the paclitaxel is administered to the patient at about 175 mg/m².

Furthermore, the paclitaxel can be administered to the patient at various frequencies. In some embodiments, the paclitaxel is administered to the patient once per week (e.g., at about 60 mg/m² once per week or about 80 mg/m² once per week). In some embodiments, the paclitaxel is administered to the patient once every three weeks (e.g., at about 135 mg/m² once every three weeks or about 175 mg/m² once every three weeks). In some embodiments, the paclitaxel is administered to the patient by intravenous infusion once per week. In some embodiments, the paclitaxel is administered to the patient by intravenous infusion once per week for three consecutive weeks. In some embodiments, the method comprises administering paclitaxel to the patient by intravenous infusion for more than one cycle, wherein each cycle comprises administering paclitaxel to the patient by intravenous infusion once per week for three consecutive weeks (e.g., once per week for three consecutive weeks at about 60 mg/m² or about 80 mg/m²). In some embodiments, the method comprises not administering paclitaxel to the patient for at least one week between each cycle.

In some embodiments, the paclitaxel is administered to the patient by infusion once every three weeks. In some embodiments, the paclitaxel is administered to the patient for about 60 minutes once per week. In some embodiments, the paclitaxel is administered to the patient for about 3 hours once per week. In some embodiments, the paclitaxel is administered to the patient for about 60 minutes once every three weeks. In some embodiments, the paclitaxel is administered to the patient for about 3 hours once every three weeks. In some embodiments, the paclitaxel is administered to the patient by intravenous infusion for about 60 minutes once per week. In some embodiments, the paclitaxel is administered to the patient by infusion for about 3 hours once per week. In some embodiments, the paclitaxel is administered to the patient by intravenous infusion for about 60 minutes once every three weeks. In some embodiments, the paclitaxel is administered to the patient by infusion for about 3 hours once every three weeks. In some embodiments, the paclitaxel is continuously administered to the patient for about 60 minutes once per week. In some embodiments, the paclitaxel is continuously administered to the patient for about 3 hours once per week. In some embodiments, the paclitaxel is continuously administered to the patient for about 60 minutes once every three weeks. In some embodiments, the paclitaxel is continuously administered to the patient for about 3 hours once every three weeks. In some embodiments, the paclitaxel is continuously administered to the patient by infusion for about 60 minutes once per week. In some embodiments, the paclitaxel is continuously administered to the patient by intravenous infusion for about 3 hours once per week. In some embodiments, the paclitaxel is continuously administered to the patient by infusion for about 60 minutes once every three weeks. In some embodiments, the paclitaxel is continuously administered to the patient by intravenous infusion for about 3 hours once every three weeks.

In embodiments of the methods of achieving at least a partial response in a patient having an ovarian cancer described herein, the disclosure also contemplates administration of the compound of Formula (I) or a pharmaceutically acceptable salt to a patient in need thereof prior to (neo-adjuvant) or after (adjuvant) surgery (e.g., surgical treatment of a solid tumor). In some embodiments, the compound of Formula (I) or a pharmaceutically acceptable salt is administered as an adjuvant. In some embodiments, the compound of Formula (I) or a pharmaceutically acceptable salt is administered as a neo-adjuvant. In some embodiments, the compound of Formula (I) or a pharmaceutically acceptable salt is administered as a neo-adjuvant and an adjuvant. In some embodiments, the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 100 years. In some embodiments, administration of the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 5 years. In some embodiments, administration of the compound of Formula (I) or a pharmaceutically acceptable salt is administered to a patient in need thereof as a neo-adjuvant for a period of 3 months to 6 months, followed by administration of the compound of Formula (I) or a pharmaceutically acceptable salt as an adjuvant for a period from 1 day to 5 years. In some embodiments, no administration of the compound of Formula (I) or a pharmaceutically acceptable salt occurs prior to surgery. In some embodiments, the compound of Formula (I) or a pharmaceutically acceptable salt is administered as an adjuvant for a period from 1 day to 100 years. In some embodiments, the compound of Formula (I) or a pharmaceutically acceptable salt is administered as an adjuvant for a period from 1 day to 5 years.

In another embodiment, provided herein is a method for achieving a stable disease condition in a patient having ovarian cancer, comprising administering to the patient an effective amount of the compound of Formula (I)

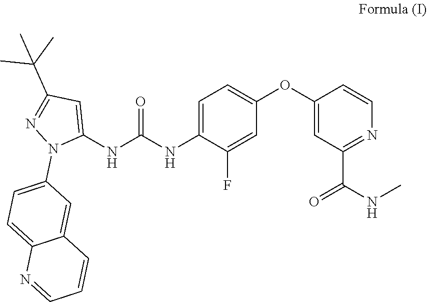 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In an embodiment, the patient has previously been administered at least one prior line of platinum-based therapy. In some embodiments, after 28 days or more of administering the compound of Formula (I) or pharmaceutically acceptable salt thereof to the patient, the patient has at least a partial response or a stable disease condition.

In some embodiments, the ovarian cancer is characterized as having a serous histology. In some embodiments, the ovarian cancer is characterized as having an endometroid histology. In some embodiments, the ovarian cancer has a mutation selected from the group consisting of a BRCA 1 positive mutation and BRCA 2 positive mutation. In some embodiments, the ovarian cancer does not have a mutation selected from the group consisting of a BRCA 1 positive mutation and BRCA 2 positive mutation. In some embodiments, the cancer is a platinum-resistant ovarian cancer.

In embodiments of the methods for achieving a stable disease condition in a patient having ovarian cancer described herein, the compound of Formula (I) or pharmaceutically acceptable salt thereof can be orally administered to the patient in need thereof. In certain embodiments, the compound of Formula (I) or pharmaceutically acceptable salt thereof is administered to the patient daily. In some embodiments, the method comprises administering to the patient about 50 mg to about 200 mg of the compound of Formula (I) or pharmaceutically acceptable salt thereof per day. In some embodiments, the method comprises administering to the patient about 50 mg to about 150 mg of the compound of Formula (I) or pharmaceutically acceptable salt thereof per day. In some embodiments, the method comprises administering to the patient about 50 mg to about 100 mg of the compound of Formula (I) or pharmaceutically acceptable salt thereof per day. In some embodiments, the method comprises administering to the patient about 60 mg to about 200 mg of the compound of Formula (I) or pharmaceutically acceptable salt thereof per day. In some embodiments, the method comprises administering to the patient about 60 mg to about 150 mg of the compound of Formula (I) or pharmaceutically acceptable salt thereof per day. In some embodiments, the method comprises administering to the patient about 60 mg to about 100 mg of the compound of Formula (I) or pharmaceutically acceptable salt thereof per day. In some embodiments, the method comprises administering to the patient about 70 mg to about 200 mg of the compound of Formula (I) or pharmaceutically acceptable salt thereof per day. In some embodiments, the method comprises administering to the patient about 70 mg to about 150 mg of the compound of Formula (I) or pharmaceutically acceptable salt thereof per day. In some embodiments, the method comprises administering to the patient about 70 mg to about 100 mg of the compound of Formula (I) or pharmaceutically acceptable salt thereof per day. In some embodiments, the method comprises administering to the patient about 80 mg to about 200 mg of the compound of Formula (I) or pharmaceutically acceptable salt thereof per day. In some embodiments, the method comprises administering to the patient about 80 mg to about 150 mg of the compound of Formula (I) or pharmaceutically acceptable salt thereof per day. In some embodiments, the method comprises administering to the patient about 80 mg to about 100 mg of the compound of Formula (I) or pharmaceutically acceptable salt thereof per day. In some embodiments, the method comprises administering to the patient about 90 mg to about 200 mg of the compound of Formula (I) or pharmaceutically acceptable salt thereof per day. In some embodiments, the method comprises administering to the patient about 90 mg to about 150 mg of the compound of Formula (I) or pharmaceutically acceptable salt thereof per day. In some embodiments, the method comprises administering to the patient about 90 mg to about 100 mg of the compound of Formula (I) or pharmaceutically acceptable salt thereof per day. In some embodiments, the method comprises administering to the patient about 100 mg to about 200 mg of the compound of Formula (I) or pharmaceutically acceptable salt thereof per day. In some embodiments, the method comprises administering to the patient about 100 mg to about 150 mg of the compound of Formula (I) or pharmaceutically acceptable salt thereof per day.

In some embodiments, the method comprises administering to the patient about 50 mg of the compound of Formula (I) or pharmaceutically acceptable salt thereof per day. In some embodiments, the method comprises administering to the patient about 55 mg of the compound of Formula (I) or pharmaceutically acceptable salt thereof per day. In some embodiments, the method comprises administering to the patient about 60 mg of the compound of Formula (I) or pharmaceutically acceptable salt thereof per day. In some embodiments, the method comprises administering to the patient about 65 mg of the compound of Formula (I) or pharmaceutically acceptable salt thereof per day. In some embodiments, the method comprises administering to the patient about 70 mg of the compound of Formula (I) or pharmaceutically acceptable salt thereof per day. In some embodiments, the method comprises administering to the patient about 75 mg of the compound of Formula (I) or pharmaceutically acceptable salt thereof per day. In some embodiments, the method comprises administering to the patient about 80 mg of the compound of Formula (I) or pharmaceutically acceptable salt thereof per day. In some embodiments, the method comprises administering to the patient about 85 mg of the compound of Formula (I) or pharmaceutically acceptable salt thereof per day. In some embodiments, the method comprises administering to the patient about 90 mg of the compound of Formula (I) or pharmaceutically acceptable salt thereof per day. In some embodiments, the method comprises administering to the patient about 95 mg of the compound of Formula (I) or pharmaceutically acceptable salt thereof per day. In some embodiments, the method comprises administering to the patient about 100 mg of the compound of Formula (I) or pharmaceutically acceptable salt thereof per day. In some embodiments, the method comprises administering to the patient about 105 mg of the compound of Formula (I) or pharmaceutically acceptable salt thereof per day. In some embodiments, the method comprises administering to the patient about 110 mg of the compound of Formula (I) or pharmaceutically acceptable salt thereof per day. In some embodiments, the method comprises administering to the patient about 115 mg of the compound of Formula (I) or pharmaceutically acceptable salt thereof per day. In some embodiments, the method comprises administering to the patient about 120 mg of the compound of Formula (I) or pharmaceutically acceptable salt thereof per day. In some embodiments, the method comprises administering to the patient about 125 mg of the compound of Formula (I) or pharmaceutically acceptable salt thereof per day. In some embodiments, the method comprises administering to the patient about 130 mg of the compound of Formula (I) or pharmaceutically acceptable salt thereof per day. In some embodiments, the method comprises administering to the patient about 135 mg of the compound of Formula (I) or pharmaceutically acceptable salt thereof per day. In some embodiments, the method comprises administering to the patient about 140 mg of the compound of Formula (I) or pharmaceutically acceptable salt thereof per day. In some embodiments, the method comprises administering to the patient about 145 mg of the compound of Formula (I) or pharmaceutically acceptable salt thereof per day. In some embodiments, the method comprises administering to the patient about 150 mg of the compound of Formula (I) or pharmaceutically acceptable salt thereof per day. In some embodiments, the method comprises administering to the patient about 155 mg of the compound of Formula (I) or pharmaceutically acceptable salt thereof per day. In some embodiments, the method comprises administering to the patient about 160 mg of the compound of Formula (I) or pharmaceutically acceptable salt thereof per day. In some embodiments, the method comprises administering to the patient about 165 mg of the compound of Formula (I) or pharmaceutically acceptable salt thereof per day. In some embodiments, the method comprises administering to the patient about 170 mg of the compound of Formula (I) or pharmaceutically acceptable salt thereof per day. In some embodiments, the method comprises administering to the patient about 175 mg of the compound of Formula (I) or pharmaceutically acceptable salt thereof per day. In some embodiments, the method comprises administering to the patient about 180 mg of the compound of Formula (I) or pharmaceutically acceptable salt thereof per day. In some embodiments, the method comprises administering to the patient about 185 mg of the compound of Formula (I) or pharmaceutically acceptable salt thereof per day. In some embodiments, the method comprises administering to the patient about 190 mg of the compound of Formula (I) or pharmaceutically acceptable salt thereof per day. In some embodiments, the method comprises administering to the patient about 195 mg of the compound of Formula (I) or pharmaceutically acceptable salt thereof per day. In some embodiments, the method comprises administering to the patient about 200 mg of the compound of Formula (I) or pharmaceutically acceptable salt thereof per day.

In some embodiments, the method comprises administering to the patient about 50 mg to about 200 mg of the compound of Formula (I) or pharmaceutically acceptable salt thereof once per day. In some embodiments, the method comprises administering to the patient about 50 mg to about 150 mg of the compound of Formula (I) or pharmaceutically acceptable salt thereof once per day. In some embodiments, the method comprises administering to the patient about 50 mg to about 100 mg of the compound of Formula (I) or pharmaceutically acceptable salt thereof once per day. In some embodiments, the method comprises administering to the patient about 60 mg to about 200 mg of the compound of Formula (I) or pharmaceutically acceptable salt thereof once per day. In some embodiments, the method comprises administering to the patient about 60 mg to about 150 mg of the compound of Formula (I) or pharmaceutically acceptable salt thereof once per day. In some embodiments, the method comprises administering to the patient about 60 mg to about 100 mg of the compound of Formula (I) or pharmaceutically acceptable salt thereof once per day. In some embodiments, the method comprises administering to the patient about 70 mg to about 200 mg of the compound of Formula (I) or pharmaceutically acceptable salt thereof once per day. In some embodiments, the method comprises administering to the patient about 70 mg to about 150 mg of the compound of Formula (I) or pharmaceutically acceptable salt thereof once per day. In some embodiments, the method comprises administering to the patient about 70 mg to about 100 mg of the compound of Formula (I) or pharmaceutically acceptable salt thereof once per day. In some embodiments, the method comprises administering to the patient about 80 mg to about 200 mg of the compound of Formula (I) or pharmaceutically acceptable salt thereof once per day. In some embodiments, the method comprises administering to the patient about 80 mg to about 150 mg of the compound of Formula (I) or pharmaceutically acceptable salt thereof once per day. In some embodiments, the method comprises administering to the patient about 80 mg to about 100 mg of the compound of Formula (I) or pharmaceutically acceptable salt thereof once per day. In some embodiments, the method comprises administering to the patient about 90 mg to about 200 mg of the compound of Formula (I) or pharmaceutically acceptable salt thereof once per day. In some embodiments, the method comprises administering to the patient about 90 mg to about 150 mg of the compound of Formula (I) or pharmaceutically acceptable salt thereof once per day. In some embodiments, the method comprises administering to the patient about 90 mg to about 100 mg of the compound of Formula (I) or pharmaceutically acceptable salt thereof once per day. In some embodiments, the method comprises administering to the patient about 100 mg to about 200 mg of the compound of Formula (I) or pharmaceutically acceptable salt thereof once per day. In some embodiments, the method comprises administering to the patient about 100 mg to about 150 mg of the compound of Formula (I) or pharmaceutically acceptable salt thereof once per day.

In some embodiments, the method comprises administering to the patient about 50 mg of the compound of Formula (I) or pharmaceutically acceptable salt thereof once per day. In some embodiments, the method comprises administering to the patient about 55 mg of the compound of Formula (I) or pharmaceutically acceptable salt thereof once per day. In some embodiments, the method comprises administering to the patient about 60 mg of the compound of Formula (I) or pharmaceutically acceptable salt thereof once per day. In some embodiments, the method comprises administering to the patient about 65 mg of the compound of Formula (I) or pharmaceutically acceptable salt thereof once per day. In some embodiments, the method comprises administering to the patient about 70 mg of the compound of Formula (I) or pharmaceutically acceptable salt thereof once per day. In some embodiments, the method comprises administering to the patient about 75 mg of the compound of Formula (I) or pharmaceutically acceptable salt thereof once per day. In some embodiments, the method comprises administering to the patient about 80 mg of the compound of Formula (I) or pharmaceutically acceptable salt thereof once per day. In some embodiments, the method comprises administering to the patient about 85 mg of the compound of Formula (I) or pharmaceutically acceptable salt thereof once per day. In some embodiments, the method comprises administering to the patient about 90 mg of the compound of Formula (I) or pharmaceutically acceptable salt thereof once per day. In some embodiments, the method comprises administering to the patient about 95 mg of the compound of Formula (I) or pharmaceutically acceptable salt thereof once per day. In some embodiments, the method comprises administering to the patient about 100 mg of the compound of Formula (I) or pharmaceutically acceptable salt thereof once per day. In some embodiments, the method comprises administering to the patient about 105 mg of the compound of Formula (I) or pharmaceutically acceptable salt thereof once per day. In some embodiments, the method comprises administering to the patient about 110 mg of the compound of Formula (I) or pharmaceutically acceptable salt thereof once per day. In some embodiments, the method comprises administering to the patient about 115 mg of the compound of Formula (I) or pharmaceutically acceptable salt thereof once per day. In some embodiments, the method comprises administering to the patient about 120 mg of the compound of Formula (I) or pharmaceutically acceptable salt thereof once per day. In some embodiments, the method comprises administering to the patient about 125 mg of the compound of Formula (I) or pharmaceutically acceptable salt thereof once per day. In some embodiments, the method comprises administering to the patient about 130 mg of the compound of Formula (I) or pharmaceutically acceptable salt thereof once per day. In some embodiments, the method comprises administering to the patient about 135 mg of the compound of Formula (I) or pharmaceutically acceptable salt thereof once per day. In some embodiments, the method comprises administering to the patient about 140 mg of the compound of Formula (I) or pharmaceutically acceptable salt thereof once per day. In some embodiments, the method comprises administering to the patient about 145 mg of the compound of Formula (I) or pharmaceutically acceptable salt thereof once per day. In some embodiments, the method comprises administering to the patient about 150 mg of the compound of Formula (I) or pharmaceutically acceptable salt thereof once per day. In some embodiments, the method comprises administering to the patient about 155 mg of the compound of Formula (I) or pharmaceutically acceptable salt thereof once per day. In some embodiments, the method comprises administering to the patient about 160 mg of the compound of Formula (I) or pharmaceutically acceptable salt thereof once per day. In some embodiments, the method comprises administering to the patient about 165 mg of the compound of Formula (I) or pharmaceutically acceptable salt thereof once per day. In some embodiments, the method comprises administering to the patient about 170 mg of the compound of Formula (I) or pharmaceutically acceptable salt thereof once per day. In some embodiments, the method comprises administering to the patient about 175 mg of the compound of Formula (I) or pharmaceutically acceptable salt thereof once per day. In some embodiments, the method comprises administering to the patient about 180 mg of the compound of Formula (I) or pharmaceutically acceptable salt thereof once per day. In some embodiments, the method comprises administering to the patient about 185 mg of the compound of Formula (I) or pharmaceutically acceptable salt thereof once per day. In some embodiments, the method comprises administering to the patient about 190 mg of the compound of Formula (I) or pharmaceutically acceptable salt thereof once per day. In some embodiments, the method comprises administering to the patient about 195 mg of the compound of Formula (I) or pharmaceutically acceptable salt thereof once per day. In some embodiments, the method comprises administering to the patient about 200 mg of the compound of Formula (I) or pharmaceutically acceptable salt thereof once per day.

In some embodiments, the method comprises administering to the patient about 50 mg to about 100 mg of the compound of Formula (I) or pharmaceutically acceptable salt thereof twice per day. In some embodiments, the method comprises administering to the patient about 60 mg to about 100 mg of the compound of Formula (I) or pharmaceutically acceptable salt thereof twice per day. In some embodiments, the method comprises administering to the patient about 70 mg to about 100 mg of the compound of Formula (I) or pharmaceutically acceptable salt thereof twice per day. In some embodiments, the method comprises administering to the patient about 80 mg to about 100 mg of the compound of Formula (I) or pharmaceutically acceptable salt thereof twice per day. In some embodiments, the method comprises administering to the patient about 90 mg to about 100 mg of the compound of Formula (I) or pharmaceutically acceptable salt thereof twice per day.

In some embodiments, the method comprises administering to the patient about 50 mg of the compound of Formula (I) or pharmaceutically acceptable salt thereof twice per day. In some embodiments, the method comprises administering to the patient about 55 mg of the compound of Formula (I) or pharmaceutically acceptable salt thereof twice per day. In some embodiments, the method comprises administering to the patient about 60 mg of the compound of Formula (I) or pharmaceutically acceptable salt thereof twice per day. In some embodiments, the method comprises administering to the patient about 65 mg of the compound of Formula (I) or pharmaceutically acceptable salt thereof twice per day. In some embodiments, the method comprises administering to the patient about 70 mg of the compound of Formula (I) or pharmaceutically acceptable salt thereof twice per day. In some embodiments, the method comprises administering to the patient about 75 mg of the compound of Formula (I) or pharmaceutically acceptable salt thereof twice per day. In some embodiments, the method comprises administering to the patient about 80 mg of the compound of Formula (I) or pharmaceutically acceptable salt thereof twice per day. In some embodiments, the method comprises administering to the patient about 85 mg of the compound of Formula (I) or pharmaceutically acceptable salt thereof twice per day. In some embodiments, the method comprises administering to the patient about 90 mg of the compound of Formula (I) or pharmaceutically acceptable salt thereof twice per day. In some embodiments, the method comprises administering to the patient about 95 mg of the compound of Formula (I) or pharmaceutically acceptable salt thereof twice per day. In some embodiments, the method comprises administering to the patient about 100 mg of the compound of Formula (I) or pharmaceutically acceptable salt thereof twice per day.

The compound of Formula (I) or pharmaceutically acceptable salt thereof may, in another embodiment, be administered to the patient on an intermittent basis in the methods of achieving a stable disease condition in a patient having ovarian cancer described herein. In some embodiments, the compound of Formula (I) or pharmaceutically acceptable salt thereof is administered the patient every other day. In some embodiments, the compound of Formula (I) or pharmaceutically acceptable salt thereof is administered the patient every other day. In some embodiments, the compound of Formula (I) or pharmaceutically acceptable salt thereof is administered the patient once per week. In some embodiments, the compound of Formula (I) or pharmaceutically acceptable salt thereof is administered the patient twice per week. In some embodiments, the compound of Formula (I) or pharmaceutically acceptable salt thereof is administered the patient three times per week.

In some embodiments, the method comprises administering to the patient about 50 mg to about 200 mg of the compound of Formula (I) or pharmaceutically acceptable salt thereof once a week. In some embodiments, the method comprises administering to the patient about 50 mg to about 150 mg of the compound of Formula (I) or pharmaceutically acceptable salt thereof once a week. In some embodiments, the method comprises administering to the patient about 50 mg to about 100 mg of the compound of Formula (I) or pharmaceutically acceptable salt thereof once a week. In some embodiments, the method comprises administering to the patient about 60 mg to about 200 mg of the compound of Formula (I) or pharmaceutically acceptable salt thereof once a week. In some embodiments, the method comprises administering to the patient about 60 mg to about 150 mg of the compound of Formula (I) or pharmaceutically acceptable salt thereof once a week. In some embodiments, the method comprises administering to the patient about 60 mg to about 100 mg of the compound of Formula (I) or pharmaceutically acceptable salt thereof once a week. In some embodiments, the method comprises administering to the patient about 70 mg to about 200 mg of the compound of Formula (I) or pharmaceutically acceptable salt thereof once a week. In some embodiments, the method comprises administering to the patient about 70 mg to about 150 mg of the compound of Formula (I) or pharmaceutically acceptable salt thereof once a week. In some embodiments, the method comprises administering to the patient about 70 mg to about 100 mg of the compound of Formula (I) or pharmaceutically acceptable salt thereof once a week. In some embodiments, the method comprises administering to the patient about 80 mg to about 200 mg of the compound of Formula (I) or pharmaceutically acceptable salt thereof once a week. In some embodiments, the method comprises administering to the patient about 80 mg to about 150 mg of the compound of Formula (I) or pharmaceutically acceptable salt thereof once a week. In some embodiments, the method comprises administering to the patient about 80 mg to about 100 mg of the compound of Formula (I) or pharmaceutically acceptable salt thereof once a week. In some embodiments, the method comprises administering to the patient about 90 mg to about 200 mg of the compound of Formula (I) or pharmaceutically acceptable salt thereof once a week. In some embodiments, the method comprises administering to the patient about 90 mg to about 150 mg of the compound of Formula (I) or pharmaceutically acceptable salt thereof once a week. In some embodiments, the method comprises administering to the patient about 90 mg to about 100 mg of the compound of Formula (I) or pharmaceutically acceptable salt thereof once a week. In some embodiments, the method comprises administering to the patient about 100 mg to about 200 mg of the compound of Formula (I) or pharmaceutically acceptable salt thereof once a week. In some embodiments, the method comprises administering to the patient about 100 mg to about 150 mg of the compound of Formula (I) or pharmaceutically acceptable salt thereof once a week.

In some embodiments, the method comprises administering to the patient about 50 mg of the compound of Formula (I) or pharmaceutically acceptable salt thereof once a week. In some embodiments, the method comprises administering to the patient about 55 mg of the compound of Formula (I) or pharmaceutically acceptable salt thereof once a week. In some embodiments, the method comprises administering to the patient about 60 mg of the compound of Formula (I) or pharmaceutically acceptable salt thereof once a week. In some embodiments, the method comprises administering to the patient about 65 mg of the compound of Formula (I) or pharmaceutically acceptable salt thereof once a week. In some embodiments, the method comprises administering to the patient about 70 mg of the compound of Formula (I) or pharmaceutically acceptable salt thereof once a week. In some embodiments, the method comprises administering to the patient about 75 mg of the compound of Formula (I) or pharmaceutically acceptable salt thereof once a week. In some embodiments, the method comprises administering to the patient about 80 mg of the compound of Formula (I) or pharmaceutically acceptable salt thereof once a week. In some embodiments, the method comprises administering to the patient about 85 mg of the compound of Formula (I) or pharmaceutically acceptable salt thereof once a week. In some embodiments, the method comprises administering to the patient about 90 mg of the compound of Formula (I) or pharmaceutically acceptable salt thereof once a week. In some embodiments, the method comprises administering to the patient about 95 mg of the compound of Formula (I) or pharmaceutically acceptable salt thereof once a week. In some embodiments, the method comprises administering to the patient about 100 mg of the compound of Formula (I) or pharmaceutically acceptable salt thereof once a week. In some embodiments, the method comprises administering to the patient about 105 mg of the compound of Formula (I) or pharmaceutically acceptable salt thereof once a week. In some embodiments, the method comprises administering to the patient about 110 mg of the compound of Formula (I) or pharmaceutically acceptable salt thereof once a week. In some embodiments, the method comprises administering to the patient about 115 mg of the compound of Formula (I) or pharmaceutically acceptable salt thereof once a week. In some embodiments, the method comprises administering to the patient about 120 mg of the compound of Formula (I) or pharmaceutically acceptable salt thereof once a week. In some embodiments, the method comprises administering to the patient about 125 mg of the compound of Formula (I) or pharmaceutically acceptable salt thereof once a week. In some embodiments, the method comprises administering to the patient about 130 mg of the compound of Formula (I) or pharmaceutically acceptable salt thereof once a week. In some embodiments, the method comprises administering to the patient about 135 mg of the compound of Formula (I) or pharmaceutically acceptable salt thereof once a week. In some embodiments, the method comprises administering to the patient about 140 mg of the compound of Formula (I) or pharmaceutically acceptable salt thereof once a week. In some embodiments, the method comprises administering to the patient about 145 mg of the compound of Formula (I) or pharmaceutically acceptable salt thereof once a week. In some embodiments, the method comprises administering to the patient about 150 mg of the compound of Formula (I) or pharmaceutically acceptable salt thereof once a week. In some embodiments, the method comprises administering to the patient about 155 mg of the compound of Formula (I) or pharmaceutically acceptable salt thereof once a week. In some embodiments, the method comprises administering to the patient about 160 mg of the compound of Formula (I) or pharmaceutically acceptable salt thereof once a week. In some embodiments, the method comprises administering to the patient about 165 mg of the compound of Formula (I) or pharmaceutically acceptable salt thereof once a week. In some embodiments, the method comprises administering to the patient about 170 mg of the compound of Formula (I) or pharmaceutically acceptable salt thereof once a week. In some embodiments, the method comprises administering to the patient about 175 mg of the compound of Formula (I) or pharmaceutically acceptable salt thereof once a week. In some embodiments, the method comprises administering to the patient about 180 mg of the compound of Formula (I) or pharmaceutically acceptable salt thereof once a week. In some embodiments, the method comprises administering to the patient about 185 mg of the compound of Formula (I) or pharmaceutically acceptable salt thereof once a week. In some embodiments, the method comprises administering to the patient about 190 mg of the compound of Formula (I) or pharmaceutically acceptable salt thereof once a week. In some embodiments, the method comprises administering to the patient about 195 mg of the compound of Formula (I) or pharmaceutically acceptable salt thereof once a week. In some embodiments, the method comprises administering to the patient about 200 mg of the compound of Formula (I) or pharmaceutically acceptable salt thereof once a week.

In some embodiments, the method comprises administering to the patient about 50 mg to about 200 mg of the compound of Formula (I) or pharmaceutically acceptable salt thereof twice a week. In some embodiments, the method comprises administering to the patient about 50 mg to about 150 mg of the compound of Formula (I) or pharmaceutically acceptable salt thereof twice a week. In some embodiments, the method comprises administering to the patient about 50 mg to about 100 mg of the compound of Formula (I) or pharmaceutically acceptable salt thereof twice a week. In some embodiments, the method comprises administering to the patient about 60 mg to about 200 mg of the compound of Formula (I) or pharmaceutically acceptable salt thereof twice a week. In some embodiments, the method comprises administering to the patient about 60 mg to about 150 mg of the compound of Formula (I) or pharmaceutically acceptable salt thereof twice a week. In some embodiments, the method comprises administering to the patient about 60 mg to about 100 mg of the compound of Formula (I) or pharmaceutically acceptable salt thereof twice a week. In some embodiments, the method comprises administering to the patient about 70 mg to about 200 mg of the compound of Formula (I) or pharmaceutically acceptable salt thereof twice a week. In some embodiments, the method comprises administering to the patient about 70 mg to about 150 mg of the compound of Formula (I) or pharmaceutically acceptable salt thereof twice a week. In some embodiments, the method comprises administering to the patient about 70 mg to about 100 mg of the compound of Formula (I) or pharmaceutically acceptable salt thereof twice a week. In some embodiments, the method comprises administering to the patient about 80 mg to about 200 mg of the compound of Formula (I) or pharmaceutically acceptable salt thereof twice a week. In some embodiments, the method comprises administering to the patient about 80 mg to about 150 mg of the compound of Formula (I) or pharmaceutically acceptable salt thereof twice a week. In some embodiments, the method comprises administering to the patient about 80 mg to about 100 mg of the compound of Formula (I) or pharmaceutically acceptable salt thereof twice a week. In some embodiments, the method comprises administering to the patient about 90 mg to about 200 mg of the compound of Formula (I) or pharmaceutically acceptable salt thereof twice a week. In some embodiments, the method comprises administering to the patient about 90 mg to about 150 mg of the compound of Formula (I) or pharmaceutically acceptable salt thereof twice a week. In some embodiments, the method comprises administering to the patient about 90 mg to about 100 mg of the compound of Formula (I) or pharmaceutically acceptable salt thereof twice a week. In some embodiments, the method comprises administering to the patient about 100 mg to about 200 mg of the compound of Formula (I) or pharmaceutically acceptable salt thereof twice a week. In some embodiments, the method comprises administering to the patient about 100 mg to about 150 mg of the compound of Formula (I) or pharmaceutically acceptable salt thereof twice a week.

In some embodiments, the method comprises administering to the patient about 50 mg of the compound of Formula (I) or pharmaceutically acceptable salt thereof twice a week. In some embodiments, the method comprises administering to the patient about 55 mg of the compound of Formula (I) or pharmaceutically acceptable salt thereof twice a week. In some embodiments, the method comprises administering to the patient about 60 mg of the compound of Formula (I) or pharmaceutically acceptable salt thereof twice a week. In some embodiments, the method comprises administering to the patient about 65 mg of the compound of Formula (I) or pharmaceutically acceptable salt thereof twice a week. In some embodiments, the method comprises administering to the patient about 70 mg of the compound of Formula (I) or pharmaceutically acceptable salt thereof twice a week. In some embodiments, the method comprises administering to the patient about 75 mg of the compound of Formula (I) or pharmaceutically acceptable salt thereof twice a week. In some embodiments, the method comprises administering to the patient about 80 mg of the compound of Formula (I) or pharmaceutically acceptable salt thereof twice a week. In some embodiments, the method comprises administering to the patient about 85 mg of the compound of Formula (I) or pharmaceutically acceptable salt thereof twice a week. In some embodiments, the method comprises administering to the patient about 90 mg of the compound of Formula (I) or pharmaceutically acceptable salt thereof twice a week. In some embodiments, the method comprises administering to the patient about 95 mg of the compound of Formula (I) or pharmaceutically acceptable salt thereof twice a week. In some embodiments, the method comprises administering to the patient about 100 mg of the compound of Formula (I) or pharmaceutically acceptable salt thereof twice a week. In some embodiments, the method comprises administering to the patient about 105 mg of the compound of Formula (I) or pharmaceutically acceptable salt thereof twice a week. In some embodiments, the method comprises administering to the patient about 110 mg of the compound of Formula (I) or pharmaceutically acceptable salt thereof twice a week. In some embodiments, the method comprises administering to the patient about 115 mg of the compound of Formula (I) or pharmaceutically acceptable salt thereof twice a week. In some embodiments, the method comprises administering to the patient about 120 mg of the compound of Formula (I) or pharmaceutically acceptable salt thereof twice a week. In some embodiments, the method comprises administering to the patient about 125 mg of the compound of Formula (I) or pharmaceutically acceptable salt thereof twice a week. In some embodiments, the method comprises administering to the patient about 130 mg of the compound of Formula (I) or pharmaceutically acceptable salt thereof twice a week. In some embodiments, the method comprises administering to the patient about 135 mg of the compound of Formula (I) or pharmaceutically acceptable salt thereof twice a week. In some embodiments, the method comprises administering to the patient about 140 mg of the compound of Formula (I) or pharmaceutically acceptable salt thereof twice a week. In some embodiments, the method comprises administering to the patient about 145 mg of the compound of Formula (I) or pharmaceutically acceptable salt thereof twice a week. In some embodiments, the method comprises administering to the patient about 150 mg of the compound of Formula (I) or pharmaceutically acceptable salt thereof twice a week. In some embodiments, the method comprises administering to the patient about 155 mg of the compound of Formula (I) or pharmaceutically acceptable salt thereof twice a week. In some embodiments, the method comprises administering to the patient about 160 mg of the compound of Formula (I) or pharmaceutically acceptable salt thereof twice a week. In some embodiments, the method comprises administering to the patient about 165 mg of the compound of Formula (I) or pharmaceutically acceptable salt thereof twice a week. In some embodiments, the method comprises administering to the patient about 170 mg of the compound of Formula (I) or pharmaceutically acceptable salt thereof twice a week. In some embodiments, the method comprises administering to the patient about 175 mg of the compound of Formula (I) or pharmaceutically acceptable salt thereof twice a week. In some embodiments, the method comprises administering to the patient about 180 mg of the compound of Formula (I) or pharmaceutically acceptable salt thereof twice a week. In some embodiments, the method comprises administering to the patient about 185 mg of the compound of Formula (I) or pharmaceutically acceptable salt thereof twice a week. In some embodiments, the method comprises administering to the patient about 190 mg of the compound of Formula (I) or pharmaceutically acceptable salt thereof twice a week. In some embodiments, the method comprises administering to the patient about 195 mg of the compound of Formula (I) or pharmaceutically acceptable salt thereof twice a week. In some embodiments, the method comprises administering to the patient about 200 mg of the compound of Formula (I) or pharmaceutically acceptable salt thereof twice a week.

In some embodiments, the method comprises administering to the patient about 50 mg to about 200 mg of the compound of Formula (I) or pharmaceutically acceptable salt thereof three times a week. In some embodiments, the method comprises administering to the patient about 50 mg to about 150 mg of the compound of Formula (I) or pharmaceutically acceptable salt thereof three times a week. In some embodiments, the method comprises administering to the patient about 50 mg to about 100 mg of the compound of Formula (I) or pharmaceutically acceptable salt thereof three times a week. In some embodiments, the method comprises administering to the patient about 60 mg to about 200 mg of the compound of Formula (I) or pharmaceutically acceptable salt thereof three times a week. In some embodiments, the method comprises administering to the patient about 60 mg to about 150 mg of the compound of Formula (I) or pharmaceutically acceptable salt thereof three times a week. In some embodiments, the method comprises administering to the patient about 60 mg to about 100 mg of the compound of Formula (I) or pharmaceutically acceptable salt thereof three times a week. In some embodiments, the method comprises administering to the patient about 70 mg to about 200 mg of the compound of Formula (I) or pharmaceutically acceptable salt thereof three times a week. In some embodiments, the method comprises administering to the patient about 70 mg to about 150 mg of the compound of Formula (I) or pharmaceutically acceptable salt thereof three times a week. In some embodiments, the method comprises administering to the patient about 70 mg to about 100 mg of the compound of Formula (I) or pharmaceutically acceptable salt thereof three times a week. In some embodiments, the method comprises administering to the patient about 80 mg to about 200 mg of the compound of Formula (I) or pharmaceutically acceptable salt thereof three times a week. In some embodiments, the method comprises administering to the patient about 80 mg to about 150 mg of the compound of Formula (I) or pharmaceutically acceptable salt thereof three times a week. In some embodiments, the method comprises administering to the patient about 80 mg to about 100 mg of the compound of Formula (I) or pharmaceutically acceptable salt thereof three times a week. In some embodiments, the method comprises administering to the patient about 90 mg to about 200 mg of the compound of Formula (I) or pharmaceutically acceptable salt thereof three times a week. In some embodiments, the method comprises administering to the patient about 90 mg to about 150 mg of the compound of Formula (I) or pharmaceutically acceptable salt thereof three times a week. In some embodiments, the method comprises administering to the patient about 90 mg to about 100 mg of the compound of Formula (I) or pharmaceutically acceptable salt thereof three times a week. In some embodiments, the method comprises administering to the patient about 100 mg to about 200 mg of the compound of Formula (I) or pharmaceutically acceptable salt thereof three times a week. In some embodiments, the method comprises administering to the patient about 100 mg to about 150 mg of the compound of Formula (I) or pharmaceutically acceptable salt thereof three times a week.

In some embodiments, the method comprises administering to the patient about 50 mg of the compound of Formula (I) or pharmaceutically acceptable salt thereof three times a week. In some embodiments, the method comprises administering to the patient about 55 mg of the compound of Formula (I) or pharmaceutically acceptable salt thereof three times a week. In some embodiments, the method comprises administering to the patient about 60 mg of the compound of Formula (I) or pharmaceutically acceptable salt thereof three times a week. In some embodiments, the method comprises administering to the patient about 65 mg of the compound of Formula (I) or pharmaceutically acceptable salt thereof three times a week. In some embodiments, the method comprises administering to the patient about 70 mg of the compound of Formula (I) or pharmaceutically acceptable salt thereof three times a week. In some embodiments, the method comprises administering to the patient about 75 mg of the compound of Formula (I) or pharmaceutically acceptable salt thereof three times a week. In some embodiments, the method comprises administering to the patient about 80 mg of the compound of Formula (I) or pharmaceutically acceptable salt thereof three times a week. In some embodiments, the method comprises administering to the patient about 85 mg of the compound of Formula (I) or pharmaceutically acceptable salt thereof three times a week. In some embodiments, the method comprises administering to the patient about 90 mg of the compound of Formula (I) or pharmaceutically acceptable salt thereof three times a week. In some embodiments, the method comprises administering to the patient about 95 mg of the compound of Formula (I) or pharmaceutically acceptable salt thereof three times a week. In some embodiments, the method comprises administering to the patient about 100 mg of the compound of Formula (I) or pharmaceutically acceptable salt thereof three times a week. In some embodiments, the method comprises administering to the patient about 105 mg of the compound of Formula (I) or pharmaceutically acceptable salt thereof three times a week. In some embodiments, the method comprises administering to the patient about 110 mg of the compound of Formula (I) or pharmaceutically acceptable salt thereof three times a week. In some embodiments, the method comprises administering to the patient about 115 mg of the compound of Formula (I) or pharmaceutically acceptable salt thereof three times a week. In some embodiments, the method comprises administering to the patient about 120 mg of the compound of Formula (I) or pharmaceutically acceptable salt thereof three times a week. In some embodiments, the method comprises administering to the patient about 125 mg of the compound of Formula (I) or pharmaceutically acceptable salt thereof three times a week. In some embodiments, the method comprises administering to the patient about 130 mg of the compound of Formula (I) or pharmaceutically acceptable salt thereof three times a week. In some embodiments, the method comprises administering to the patient about 135 mg of the compound of Formula (I) or pharmaceutically acceptable salt thereof three times a week. In some embodiments, the method comprises administering to the patient about 140 mg of the compound of Formula (I) or pharmaceutically acceptable salt thereof three times a week. In some embodiments, the method comprises administering to the patient about 145 mg of the compound of Formula (I) or pharmaceutically acceptable salt thereof three times a week. In some embodiments, the method comprises administering to the patient about 150 mg of the compound of Formula (I) or pharmaceutically acceptable salt thereof three times a week. In some embodiments, the method comprises administering to the patient about 155 mg of the compound of Formula (I) or pharmaceutically acceptable salt thereof three times a week. In some embodiments, the method comprises administering to the patient about 160 mg of the compound of Formula (I) or pharmaceutically acceptable salt thereof three times a week. In some embodiments, the method comprises administering to the patient about 165 mg of the compound of Formula (I) or pharmaceutically acceptable salt thereof three times a week. In some embodiments, the method comprises administering to the patient about 170 mg of the compound of Formula (I) or pharmaceutically acceptable salt thereof three times a week. In some embodiments, the method comprises administering to the patient about 175 mg of the compound of Formula (I) or pharmaceutically acceptable salt thereof three times a week. In some embodiments, the method comprises administering to the patient about 180 mg of the compound of Formula (I) or pharmaceutically acceptable salt thereof three times a week. In some embodiments, the method comprises administering to the patient about 185 mg of the compound of Formula (I) or pharmaceutically acceptable salt thereof three times a week. In some embodiments, the method comprises administering to the patient about 190 mg of the compound of Formula (I) or pharmaceutically acceptable salt thereof three times a week. In some embodiments, the method comprises administering to the patient about 195 mg of the compound of Formula (I) or pharmaceutically acceptable salt thereof three times a week. In some embodiments, the method comprises administering to the patient about 200 mg of the compound of Formula (I) or pharmaceutically acceptable salt thereof three times a week.

In some embodiments, the method comprises administering to the patient about 50 mg to about 200 mg of the compound of Formula (I) or pharmaceutically acceptable salt thereof every other day. In some embodiments, the method comprises administering to the patient about 50 mg to about 150 mg of the compound of Formula (I) or pharmaceutically acceptable salt thereof every other day. In some embodiments, the method comprises administering to the patient about 50 mg to about 100 mg of the compound of Formula (I) or pharmaceutically acceptable salt thereof every other day. In some embodiments, the method comprises administering to the patient about 60 mg to about 200 mg of the compound of Formula (I) or pharmaceutically acceptable salt thereof every other day. In some embodiments, the method comprises administering to the patient about 60 mg to about 150 mg of the compound of Formula (I) or pharmaceutically acceptable salt thereof every other day. In some embodiments, the method comprises administering to the patient about 60 mg to about 100 mg of the compound of Formula (I) or pharmaceutically acceptable salt thereof every other day. In some embodiments, the method comprises administering to the patient about 70 mg to about 200 mg of the compound of Formula (I) or pharmaceutically acceptable salt thereof every other day. In some embodiments, the method comprises administering to the patient about 70 mg to about 150 mg of the compound of Formula (I) or pharmaceutically acceptable salt thereof every other day. In some embodiments, the method comprises administering to the patient about 70 mg to about 100 mg of the compound of Formula (I) or pharmaceutically acceptable salt thereof every other day. In some embodiments, the method comprises administering to the patient about 80 mg to about 200 mg of the compound of Formula (I) or pharmaceutically acceptable salt thereof every other day. In some embodiments, the method comprises administering to the patient about 80 mg to about 150 mg of the compound of Formula (I) or pharmaceutically acceptable salt thereof every other day. In some embodiments, the method comprises administering to the patient about 80 mg to about 100 mg of the compound of Formula (I) or pharmaceutically acceptable salt thereof every other day. In some embodiments, the method comprises administering to the patient about 90 mg to about 200 mg of the compound of Formula (I) or pharmaceutically acceptable salt thereof every other day. In some embodiments, the method comprises administering to the patient about 90 mg to about 150 mg of the compound of Formula (I) or pharmaceutically acceptable salt thereof every other day. In some embodiments, the method comprises administering to the patient about 90 mg to about 100 mg of the compound of Formula (I) or pharmaceutically acceptable salt thereof every other day. In some embodiments, the method comprises administering to the patient about 100 mg to about 200 mg of the compound of Formula (I) or pharmaceutically acceptable salt thereof every other day. In some embodiments, the method comprises administering to the patient about 100 mg to about 150 mg of the compound of Formula (I) or pharmaceutically acceptable salt thereof every other day.

In some embodiments, the method comprises administering to the patient about 50 mg of the compound of Formula (I) or pharmaceutically acceptable salt thereof every other day. In some embodiments, the method comprises administering to the patient about 55 mg of the compound of Formula (I) or pharmaceutically acceptable salt thereof every other day. In some embodiments, the method comprises administering to the patient about 60 mg of the compound of Formula (I) or pharmaceutically acceptable salt thereof every other day. In some embodiments, the method comprises administering to the patient about 65 mg of the compound of Formula (I) or pharmaceutically acceptable salt thereof every other day. In some embodiments, the method comprises administering to the patient about 70 mg of the compound of Formula (I) or pharmaceutically acceptable salt thereof every other day. In some embodiments, the method comprises administering to the patient about 75 mg of the compound of Formula (I) or pharmaceutically acceptable salt thereof every other day. In some embodiments, the method comprises administering to the patient about 80 mg of the compound of Formula (I) or pharmaceutically acceptable salt thereof every other day. In some embodiments, the method comprises administering to the patient about 85 mg of the compound of Formula (I) or pharmaceutically acceptable salt thereof every other day. In some embodiments, the method comprises administering to the patient about 90 mg of the compound of Formula (I) or pharmaceutically acceptable salt thereof every other day. In some embodiments, the method comprises administering to the patient about 95 mg of the compound embodiments, the method comprises administering to the patient about 100 mg of the compound of Formula (I) or pharmaceutically acceptable salt thereof every other day. In some embodiments, the method comprises administering to the patient about 105 mg of the compound of Formula (I) or pharmaceutically acceptable salt thereof every other day. In some embodiments, the method comprises administering to the patient about 110 mg of the compound of Formula (I) or pharmaceutically acceptable salt thereof every other day. In some embodiments, the method comprises administering to the patient about 115 mg of the compound of Formula (I) or pharmaceutically acceptable salt thereof every other day. In some embodiments, the method comprises administering to the patient about 120 mg of the compound of Formula (I) or pharmaceutically acceptable salt thereof every other day. In some embodiments, the method comprises administering to the patient about 125 mg of the compound of Formula (I) or pharmaceutically acceptable salt thereof every other day. In some embodiments, the method comprises administering to the patient about 130 mg of the compound of Formula (I) or pharmaceutically acceptable salt thereof every other day. In some embodiments, the method comprises administering to the patient about 135 mg of the compound of Formula (I) or pharmaceutically acceptable salt thereof every other day. In some embodiments, the method comprises administering to the patient about 140 mg of the compound of Formula (I) or pharmaceutically acceptable salt thereof every other day. In some embodiments, the method comprises administering to the patient about 145 mg of the compound of Formula (I) or pharmaceutically acceptable salt thereof every other day. In some embodiments, the method comprises administering to the patient about 150 mg of the compound of Formula (I) or pharmaceutically acceptable salt thereof every other day. In some embodiments, the method comprises administering to the patient about 155 mg of the compound of Formula (I) or pharmaceutically acceptable salt thereof every other day. In some embodiments, the method comprises administering to the patient about 160 mg of the compound of Formula (I) or pharmaceutically acceptable salt thereof every other day. In some embodiments, the method comprises administering to the patient about 165 mg of the compound of Formula (I) or pharmaceutically acceptable salt thereof every other day. In some embodiments, the method comprises administering to the patient about 170 mg of the compound of Formula (I) or pharmaceutically acceptable salt thereof every other day. In some embodiments, the method comprises administering to the patient about 175 mg of the compound of Formula (I) or pharmaceutically acceptable salt thereof every other day. In some embodiments, the method comprises administering to the patient about 180 mg of the compound of Formula (I) or pharmaceutically acceptable salt thereof every other day. In some embodiments, the method comprises administering to the patient about 185 mg of the compound of Formula (I) or pharmaceutically acceptable salt thereof every other day. In some embodiments, the method comprises administering to the patient about 190 mg of the compound of Formula (I) or pharmaceutically acceptable salt thereof every other day. In some embodiments, the method comprises administering to the patient about 195 mg of the compound of Formula (I) or pharmaceutically acceptable salt thereof every other day. In some embodiments, the method comprises administering to the patient about 200 mg of the compound of Formula (I) or pharmaceutically acceptable salt thereof every other day.

In some embodiments, a method of achieving a stable disease condition in a patient having an ovarian cancer described herein further comprises administering paclitaxel to the patient in need thereof. In some embodiments, the paclitaxel is intravenously administered to the patient, e.g., by intravenous infusion. The intravenously administered paclitaxel can be administered to a patient at various dose levels. In some embodiments, the paclitaxel is administered to the patient at about 50 mg/m² to about 150 mg/m². In some embodiments, the paclitaxel is administered to the patient at about 50 mg/m² to about 100 mg/m². In some embodiments, the paclitaxel is administered to the patient at about 60 mg/m² to about 80 mg/m². In some embodiments, the paclitaxel is administered to the patient at about 60 mg/m². In some embodiments, the paclitaxel is administered to the patient at about 80 mg/m². In some embodiments, the paclitaxel is administered to the patient at about 135 mg/m². In some embodiments, the paclitaxel is administered to the patient at about 175 mg/m².

Furthermore, the paclitaxel can be administered to the patient at various frequencies. In some embodiments, the paclitaxel is administered to the patient once per week (e.g., at about 60 mg/m² once per week or about 80 mg/m² once per week). In some embodiments, the paclitaxel is administered to the patient once every three weeks (e.g., at about 135 mg/m² once every three weeks or about 175 mg/m² once every three weeks). In some embodiments, the paclitaxel is administered to the patient by intravenous infusion once per week. In some embodiments, the paclitaxel is administered to the patient by intravenous infusion once per week for three consecutive weeks. In some embodiments, the method comprises administering paclitaxel to the patient by intravenous infusion for more than one cycle, wherein each cycle comprises administering paclitaxel to the patient by intravenous infusion once per week for three consecutive weeks (e.g., once per week for three consecutive weeks at about 60 mg/m² or about 80 mg/m²). In some embodiments, the method comprises not administering paclitaxel to the patient for at least one week between each cycle.

In some embodiments, the paclitaxel is administered to the patient by infusion once every three weeks. In some embodiments, the paclitaxel is administered to the patient for about 60 minutes once per week. In some embodiments, the paclitaxel is administered to the patient for about 3 hours once per week. In some embodiments, the paclitaxel is administered to the patient for about 60 minutes once every three weeks. In some embodiments, the paclitaxel is administered to the patient for about 3 hours once every three weeks. In some embodiments, the paclitaxel is administered to the patient by intravenous infusion for about 60 minutes once per week. In some embodiments, the paclitaxel is administered to the patient by infusion for about 3 hours once per week. In some embodiments, the paclitaxel is administered to the patient by intravenous infusion for about 60 minutes once every three weeks. In some embodiments, the paclitaxel is administered to the patient by infusion for about 3 hours once every three weeks. In some embodiments, the paclitaxel is continuously administered to the patient for about 60 minutes once per week. In some embodiments, the paclitaxel is continuously administered to the patient for about 3 hours once per week. In some embodiments, the paclitaxel is continuously administered to the patient for about 60 minutes once every three weeks. In some embodiments, the paclitaxel is continuously administered to the patient for about 3 hours once every three weeks. In some embodiments, the paclitaxel is continuously administered to the patient by infusion for about 60 minutes once per week. In some embodiments, the paclitaxel is continuously administered to the patient by intravenous infusion for about 3 hours once per week. In some embodiments, the paclitaxel is continuously administered to the patient by infusion for about 60 minutes once every three weeks. In some embodiments, the paclitaxel is continuously administered to the patient by intravenous infusion for about 3 hours once every three weeks.

In embodiments of the methods of achieving at least a stable disease condition in a patient having an ovarian cancer described herein, the disclosure also contemplates administration of the compound of Formula (I) or a pharmaceutically acceptable salt to a patient in need thereof prior to (neo-adjuvant) or after (adjuvant) surgery (e.g., surgical treatment of a solid tumor). In some embodiments, the compound of Formula (I) or a pharmaceutically acceptable salt is administered as an adjuvant. In some embodiments, the compound of Formula (I) or a pharmaceutically acceptable salt is administered as a neo-adjuvant. In some embodiments, the compound of Formula (I) or a pharmaceutically acceptable salt is administered as a neo-adjuvant and an adjuvant. In some embodiments, the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 100 years. In some embodiments, administration of the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 5 years. In some embodiments, administration of the compound of Formula (I) or a pharmaceutically acceptable salt is administered to a patient in need thereof as a neo-adjuvant for a period of 3 months to 6 months, followed by administration of the compound of Formula (I) or a pharmaceutically acceptable salt as an adjuvant for a period from 1 day to 5 years. In some embodiments, no administration of the compound of Formula (I) or a pharmaceutically acceptable salt occurs prior to surgery. In some embodiments, the compound of Formula (I) or a pharmaceutically acceptable salt is administered as an adjuvant for a period from 1 day to 100 years. In some embodiments, the compound of Formula (I) or a pharmaceutically acceptable salt is administered as an adjuvant for a period from 1 day to 5 years.

In another embodiment, described herein is a method of treating ovarian cancer in a patient in need thereof, comprising administering to the patient 50 mg, twice per day, of a compound of Formula (I):

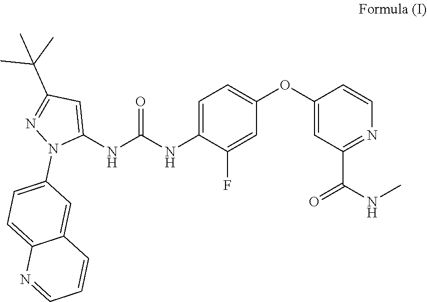 [SMILES: CNC(=O)c1cc(Oc2ccc(NC(=O)Nc3cc(C(C)(C)C)nn3-c3ccc4ncccc4c3)c(F)c2)ccn1]

and 80 mg/m² of paclitaxel once per week. In some embodiments, the patient had received at least one prior platinum therapy. In some embodiments, the ovarian cancer is a platinum-resistant ovarian cancer. In some embodiments, the ovarian cancer is characterized as having a serous histology. In some embodiments, the ovarian cancer is characterized as having an endometroid histology. In some embodiments, the ovarian cancer has a mutation selected from the group consisting of a BRCA 1 positive mutation and BRCA 2 positive mutation. In some embodiments, the ovarian cancer does not have a mutation selected from the group consisting of a BRCA 1 positive mutation and BRCA 2 positive mutation.

In another embodiment, described herein is a method of treating ovarian cancer in a patient in need thereof, comprising administering to the patient 100 mg, twice per day, of a compound of Formula (I):

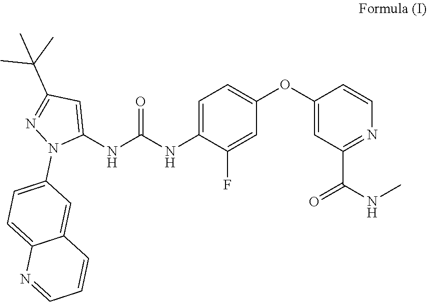 [SMILES: CNC(=O)c1cc(Oc2ccc(NC(=O)Nc3cc(C(C)(C)C)nn3-c3ccc4ncccc4c3)c(F)c2)ccn1]

and 80 mg/m² of paclitaxel once per week. In some embodiments, the patient had received at least one prior platinum therapy. In some embodiments, the ovarian cancer is a platinum-resistant ovarian cancer. In some embodiments, the ovarian cancer is characterized as having a serous histology. In some embodiments, the ovarian cancer is characterized as having an endometroid histology. In some embodiments, the ovarian cancer has a mutation selected from the group consisting of a BRCA 1 positive mutation and BRCA 2 positive mutation. In some embodiments, the ovarian cancer does not have a mutation selected from the group consisting of a BRCA 1 positive mutation and BRCA 2 positive mutation.

### Pancreatic Ductal Adenocarcinoma

Also provided herein is a method of treating pancreatic ductal adenocarcinoma in a patient in need thereof, comprising administering to the patient an effective amount of the compound of Formula (I)

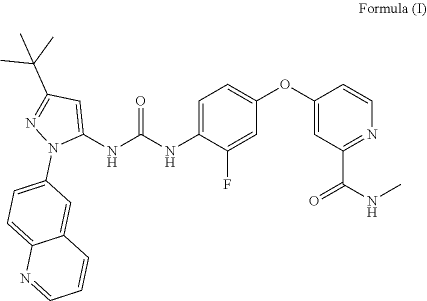 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In one embodiment, provided herein is a method of treating pancreatic ductal adenocarcinoma in a patient in need thereof, comprising administering to the patient about 50 mg to about 200 mg of the compound of Formula (I)

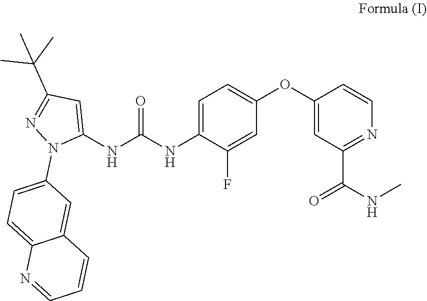 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof per day.

In embodiments of the methods of treating an pancreatic ductal adenocarcinoma described herein, the compound of Formula (I) or pharmaceutically acceptable salt thereof can be orally administered to the patient in need thereof. In certain embodiments, the compound of Formula (I) or pharmaceutically acceptable salt thereof is administered to the patient daily. In some embodiments, the method comprises administering to the patient about 50 mg to about 150 mg of the compound of Formula (I) or pharmaceutically acceptable salt thereof per day. In some embodiments, the method comprises administering to the patient about 50 mg to about 100 mg of the compound of Formula (I) or pharmaceutically acceptable salt thereof per day. In some embodiments, the method comprises administering to the patient about 60 mg to about 200 mg of the compound of Formula (I) or pharmaceutically acceptable salt thereof per day. In some embodiments, the method comprises administering to the patient about 60 mg to about 150 mg of the compound of Formula (I) or pharmaceutically acceptable salt thereof per day. In some embodiments, the method comprises administering to the patient about 60 mg to about 100 mg of the compound of Formula (I) or pharmaceutically acceptable salt thereof per day. In some embodiments, the method comprises administering to the patient about 70 mg to about 200 mg of the compound of Formula (I) or pharmaceutically acceptable salt thereof per day. In some embodiments, the method comprises administering to the patient about 70 mg to about 150 mg of the compound of Formula (I) or pharmaceutically acceptable salt thereof per day. In some embodiments, the method comprises administering to the patient about 70 mg to about 100 mg of the compound of Formula (I) or pharmaceutically acceptable salt thereof per day. In some embodiments, the method comprises administering to the patient about 80 mg to about 200 mg of the compound of Formula (I) or pharmaceutically acceptable salt thereof per day. In some embodiments, the method comprises administering to the patient about 80 mg to about 150 mg of the compound of Formula (I) or pharmaceutically acceptable salt thereof per day. In some embodiments, the method comprises administering to the patient about 80 mg to about 100 mg of the compound of Formula (I) or pharmaceutically acceptable salt thereof per day. In some embodiments, the method comprises administering to the patient about 90 mg to about 200 mg of the compound of Formula (I) or pharmaceutically acceptable salt thereof per day. In some embodiments, the method comprises administering to the patient about 90 mg to about 150 mg of the compound of Formula (I) or pharmaceutically acceptable salt thereof per day. In some embodiments, the method comprises administering to the patient about 90 mg to about 100 mg of the compound of Formula (I) or pharmaceutically acceptable salt thereof per day. In some embodiments, the method comprises administering to the patient about 100 mg to about 200 mg of the compound of Formula (I) or pharmaceutically acceptable salt thereof per day. In some embodiments, the method comprises administering to the patient about 100 mg to about 150 mg of the compound of Formula (I) or pharmaceutically acceptable salt thereof per day.

In some embodiments, the method comprises administering to the patient about 50 mg of the compound of Formula (I) or pharmaceutically acceptable salt thereof per day. In some embodiments, the method comprises administering to the patient about 55 mg of the compound of Formula (I) or pharmaceutically acceptable salt thereof per day. In some embodiments, the method comprises administering to the patient about 60 mg of the compound of Formula (I) or pharmaceutically acceptable salt thereof per day. In some embodiments, the method comprises administering to the patient about 65 mg of the compound of Formula (I) or pharmaceutically acceptable salt thereof per day. In some embodiments, the method comprises administering to the patient about 70 mg of the compound of Formula (I) or pharmaceutically acceptable salt thereof per day. In some embodiments, the method comprises administering to the patient about 75 mg of the compound of Formula (I) or pharmaceutically acceptable salt thereof per day. In some embodiments, the method comprises administering to the patient about 80 mg of the compound of Formula (I) or pharmaceutically acceptable salt thereof per day. In some embodiments, the method comprises administering to the patient about 85 mg of the compound of Formula (I) or pharmaceutically acceptable salt thereof per day. In some embodiments, the method comprises administering to the patient about 90 mg of the compound of Formula (I) or pharmaceutically acceptable salt thereof per day. In some embodiments, the method comprises administering to the patient about 95 mg of the compound of Formula (I) or pharmaceutically acceptable salt thereof per day. In some embodiments, the method comprises administering to the patient about 100 mg of the compound of Formula (I) or pharmaceutically acceptable salt thereof per day. In some embodiments, the method comprises administering to the patient about 105 mg of the compound of Formula (I) or pharmaceutically acceptable salt thereof per day. In some embodiments, the method comprises administering to the patient about 110 mg of the compound of Formula (I) or pharmaceutically acceptable salt thereof per day. In some embodiments, the method comprises administering to the patient about 115 mg of the compound of Formula (I) or pharmaceutically acceptable salt thereof per day. In some embodiments, the method comprises administering to the patient about 120 mg of the compound of Formula (I) or pharmaceutically acceptable salt thereof per day. In some embodiments, the method comprises administering to the patient about 125 mg of the compound of Formula (I) or pharmaceutically acceptable salt thereof per day. In some embodiments, the method comprises administering to the patient about 130 mg of the compound of Formula (I) or pharmaceutically acceptable salt thereof per day. In some embodiments, the method comprises administering to the patient about 135 mg of the compound of Formula (I) or pharmaceutically acceptable salt thereof per day. In some embodiments, the method comprises administering to the patient about 140 mg of the compound of Formula (I) or pharmaceutically acceptable salt thereof per day. In some embodiments, the method comprises administering to the patient about 145 mg of the compound of Formula (I) or pharmaceutically acceptable salt thereof per day. In some embodiments, the method comprises administering to the patient about 150 mg of the compound of Formula (I) or pharmaceutically acceptable salt thereof per day. In some embodiments, the method comprises administering to the patient about 155 mg of the compound of Formula (I) or pharmaceutically acceptable salt thereof per day. In some embodiments, the method comprises administering to the patient about 160 mg of the compound of Formula (I) or pharmaceutically acceptable salt thereof per day. In some embodiments, the method comprises administering to the patient about 165 mg of the compound of Formula (I) or pharmaceutically acceptable salt thereof per day. In some embodiments, the method comprises administering to the patient about 170 mg of the compound of Formula (I) or pharmaceutically acceptable salt thereof per day. In some embodiments, the method comprises administering to the patient about 175 mg of the compound of Formula (I) or pharmaceutically acceptable salt thereof per day. In some embodiments, the method comprises administering to the patient about 180 mg of the compound of Formula (I) or pharmaceutically acceptable salt thereof per day. In some embodiments, the method comprises administering to the patient about 185 mg of the compound of Formula (I) or pharmaceutically acceptable salt thereof per day. In some embodiments, the method comprises administering to the patient about 190 mg of the compound of Formula (I) or pharmaceutically acceptable salt thereof per day. In some embodiments, the method comprises administering to the patient about 195 mg of the compound of Formula (I) or pharmaceutically acceptable salt thereof per day. In some embodiments, the method comprises administering to the patient about 200 mg of the compound of Formula (I) or pharmaceutically acceptable salt thereof per day.

In some embodiments, the method comprises administering to the patient about 50 mg to about 200 mg of the compound of Formula (I) or pharmaceutically acceptable salt thereof once per day. In some embodiments, the method comprises administering to the patient about 50 mg to about 150 mg of the compound of Formula (I) or pharmaceutically acceptable salt thereof once per day. In some embodiments, the method comprises administering to the patient about 50 mg to about 100 mg of the compound of Formula (I) or pharmaceutically acceptable salt thereof once per day. In some embodiments, the method comprises administering to the patient about 60 mg to about 200 mg of the compound of Formula (I) or pharmaceutically acceptable salt thereof once per day. In some embodiments, the method comprises administering to the patient about 60 mg to about 150 mg of the compound of Formula (I) or pharmaceutically acceptable salt thereof once per day. In some embodiments, the method comprises administering to the patient about 60 mg to about 100 mg of the compound of Formula (I) or pharmaceutically acceptable salt thereof once per day. In some embodiments, the method comprises administering to the patient about 70 mg to about 200 mg of the compound of Formula (I) or pharmaceutically acceptable salt thereof once per day. In some embodiments, the method comprises administering to the patient about 70 mg to about 150 mg of the compound of Formula (I) or pharmaceutically acceptable salt thereof once per day. In some embodiments, the method comprises administering to the patient about 70 mg to about 100 mg of the compound of Formula (I) or pharmaceutically acceptable salt thereof once per day. In some embodiments, the method comprises administering to the patient about 80 mg to about 200 mg of the compound of Formula (I) or pharmaceutically acceptable salt thereof once per day. In some embodiments, the method comprises administering to the patient about 80 mg to about 150 mg of the compound of Formula (I) or pharmaceutically acceptable salt thereof once per day. In some embodiments, the method comprises administering to the patient about 80 mg to about 100 mg of the compound of Formula (I) or pharmaceutically acceptable salt thereof once per day. In some embodiments, the method comprises administering to the patient about 90 mg to about 200 mg of the compound of Formula (I) or pharmaceutically acceptable salt thereof once per day. In some embodiments, the method comprises administering to the patient about 90 mg to about 150 mg of the compound of Formula (I) or pharmaceutically acceptable salt thereof once per day. In some embodiments, the method comprises administering to the patient about 90 mg to about 100 mg of the compound of Formula (I) or pharmaceutically acceptable salt thereof once per day. In some embodiments, the method comprises administering to the patient about 100 mg to about 200 mg of the compound of Formula (I) or pharmaceutically acceptable salt thereof once per day. In some embodiments, the method comprises administering to the patient about 100 mg to about 150 mg of the compound of Formula (I) or pharmaceutically acceptable salt thereof once per day.

In some embodiments, the method comprises administering to the patient about 50 mg of the compound of Formula (I) or pharmaceutically acceptable salt thereof once per day. In some embodiments, the method comprises administering to the patient about 55 mg of the compound of Formula (I) or pharmaceutically acceptable salt thereof once per day. In some embodiments, the method comprises administering to the patient about 60 mg of the compound of Formula (I) or pharmaceutically acceptable salt thereof once per day. In some embodiments, the method comprises administering to the patient about 65 mg of the compound of Formula (I) or pharmaceutically acceptable salt thereof once per day. In some embodiments, the method comprises administering to the patient about 70 mg of the compound of Formula (I) or pharmaceutically acceptable salt thereof once per day. In some embodiments, the method comprises administering to the patient about 75 mg of the compound of Formula (I) or pharmaceutically acceptable salt thereof once per day. In some embodiments, the method comprises administering to the patient about 80 mg of the compound of Formula (I) or pharmaceutically acceptable salt thereof once per day. In some embodiments, the method comprises administering to the patient about 85 mg of the compound of Formula (I) or pharmaceutically acceptable salt thereof once per day. In some embodiments, the method comprises administering to the patient about 90 mg of the compound of Formula (I) or pharmaceutically acceptable salt thereof once per day. In some embodiments, the method comprises administering to the patient about 95 mg of the compound of Formula (I) or pharmaceutically acceptable salt thereof once per day. In some embodiments, the method comprises administering to the patient about 100 mg of the compound of Formula (I) or pharmaceutically acceptable salt thereof once per day. In some embodiments, the method comprises administering to the patient about 105 mg of the compound of Formula (I) or pharmaceutically acceptable salt thereof once per day. In some embodiments, the method comprises administering to the patient about 110 mg of the compound of Formula (I) or pharmaceutically acceptable salt thereof once per day. In some embodiments, the method comprises administering to the patient about 115 mg of the compound of Formula (I) or pharmaceutically acceptable salt thereof once per day. In some embodiments, the method comprises administering to the patient about 120 mg of the compound of Formula (I) or pharmaceutically acceptable salt thereof once per day. In some embodiments, the method comprises administering to the patient about 125 mg of the compound of Formula (I) or pharmaceutically acceptable salt thereof once per day. In some embodiments, the method comprises administering to the patient about 130 mg of the compound of Formula (I) or pharmaceutically acceptable salt thereof once per day. In some embodiments, the method comprises administering to the patient about 135 mg of the compound of Formula (I) or pharmaceutically acceptable salt thereof once per day. In some embodiments, the method comprises administering to the patient about 140 mg of the compound of Formula (I) or pharmaceutically acceptable salt thereof once per day. In some embodiments, the method comprises administering to the patient about 145 mg of the compound of Formula (I) or pharmaceutically acceptable salt thereof once per day. In some embodiments, the method comprises administering to the patient about 150 mg of the compound of Formula (I) or pharmaceutically acceptable salt thereof once per day. In some embodiments, the method comprises administering to the patient about 155 mg of the compound of Formula (I) or pharmaceutically acceptable salt thereof once per day. In some embodiments, the method comprises administering to the patient about 160 mg of the compound of Formula (I) or pharmaceutically acceptable salt thereof once per day. In some embodiments, the method comprises administering to the patient about 165 mg of the compound of Formula (I) or pharmaceutically acceptable salt thereof once per day. In some embodiments, the method comprises administering to the patient about 170 mg of the compound of Formula (I) or pharmaceutically acceptable salt thereof once per day. In some embodiments, the method comprises administering to the patient about 175 mg of the compound of Formula (I) or pharmaceutically acceptable salt thereof once per day. In some embodiments, the method comprises administering to the patient about 180 mg of the compound of Formula (I) or pharmaceutically acceptable salt thereof once per day. In some embodiments, the method comprises administering to the patient about 185 mg of the compound of Formula (I) or pharmaceutically acceptable salt thereof once per day. In some embodiments, the method comprises administering to the patient about 190 mg of the compound of Formula (I) or pharmaceutically acceptable salt thereof once per day. In some embodiments, the method comprises administering to the patient about 195 mg of the compound of Formula (I) or pharmaceutically acceptable salt thereof once per day. In some embodiments, the method comprises administering to the patient about 200 mg of the compound of Formula (I) or pharmaceutically acceptable salt thereof once per day.

In some embodiments, the method comprises administering to the patient about 50 mg to about 100 mg of the compound of Formula (I) or pharmaceutically acceptable salt thereof twice per day. In some embodiments, the method comprises administering to the patient about 60 mg to about 100 mg of the compound of Formula (I) or pharmaceutically acceptable salt thereof twice per day. In some embodiments, the method comprises administering to the patient about 70 mg to about 100 mg of the compound of Formula (I) or pharmaceutically acceptable salt thereof twice per day. In some embodiments, the method comprises administering to the patient about 80 mg to about 100 mg of the compound of Formula (I) or pharmaceutically acceptable salt thereof twice per day. In some embodiments, the method comprises administering to the patient about 90 mg to about 100 mg of the compound of Formula (I) or pharmaceutically acceptable salt thereof twice per day.

In some embodiments, the method comprises administering to the patient about 50 mg of the compound of Formula (I) or pharmaceutically acceptable salt thereof twice per day. In some embodiments, the method comprises administering to the patient about 55 mg of the compound of Formula (I) or pharmaceutically acceptable salt thereof twice per day. In some embodiments, the method comprises administering to the patient about 60 mg of the compound of Formula (I) or pharmaceutically acceptable salt thereof twice per day. In some embodiments, the method comprises administering to the patient about 65 mg of the compound of Formula (I) or pharmaceutically acceptable salt thereof twice per day. In some embodiments, the method comprises administering to the patient about 70 mg of the compound of Formula (I) or pharmaceutically acceptable salt thereof twice per day. In some embodiments, the method comprises administering to the patient about 75 mg of the compound of Formula (I) or pharmaceutically acceptable salt thereof twice per day. In some embodiments, the method comprises administering to the patient about 80 mg of the compound of Formula (I) or pharmaceutically acceptable salt thereof twice per day. In some embodiments, the method comprises administering to the patient about 85 mg of the compound of Formula (I) or pharmaceutically acceptable salt thereof twice per day. In some embodiments, the method comprises administering to the patient about 90 mg of the compound of Formula (I) or pharmaceutically acceptable salt thereof twice per day. In some embodiments, the method comprises administering to the patient about 95 mg of the compound of Formula (I) or pharmaceutically acceptable salt thereof twice per day. In some embodiments, the method comprises administering to the patient about 100 mg of the compound of Formula (I) or pharmaceutically acceptable salt thereof twice per day.

The compound of Formula (I) or pharmaceutically acceptable salt thereof may, in another embodiment, be administered to the patient on an intermittent basis in the methods of treating pancreatic ductal adenocarcinoma described herein. In some embodiments, the compound of Formula (I) or pharmaceutically acceptable salt thereof is administered the patient every other day. In some embodiments, the compound of Formula (I) or pharmaceutically acceptable salt thereof is administered the patient every other day. In some embodiments, the compound of Formula (I) or pharmaceutically acceptable salt thereof is administered the patient once per week. In some embodiments, the compound of Formula (I) or pharmaceutically acceptable salt thereof is administered the patient twice per week. In some embodiments, the compound of Formula (I) or pharmaceutically acceptable salt thereof is administered the patient three times per week.

In some embodiments, the method comprises administering to the patient about 50 mg to about 200 mg of the compound of Formula (I) or pharmaceutically acceptable salt thereof once a week. In some embodiments, the method comprises administering to the patient about 50 mg to about 150 mg of the compound of Formula (I) or pharmaceutically acceptable salt thereof once a week. In some embodiments, the method comprises administering to the patient about 50 mg to about 100 mg of the compound of Formula (I) or pharmaceutically acceptable salt thereof once a week. In some embodiments, the method comprises administering to the patient about 60 mg to about 200 mg of the compound of Formula (I) or pharmaceutically acceptable salt thereof once a week. In some embodiments, the method comprises administering to the patient about 60 mg to about 150 mg of the compound of Formula (I) or pharmaceutically acceptable salt thereof once a week. In some embodiments, the method comprises administering to the patient about 60 mg to about 100 mg of the compound of Formula (I) or pharmaceutically acceptable salt thereof once a week. In some embodiments, the method comprises administering to the patient about 70 mg to about 200 mg of the compound of Formula (I) or pharmaceutically acceptable salt thereof once a week. In some embodiments, the method comprises administering to the patient about 70 mg to about 150 mg of the compound of Formula (I) or pharmaceutically acceptable salt thereof once a week. In some embodiments, the method comprises administering to the patient about 70 mg to about 100 mg of the compound of Formula (I) or pharmaceutically acceptable salt thereof once a week. In some embodiments, the method comprises administering to the patient about 80 mg to about 200 mg of the compound of Formula (I) or pharmaceutically acceptable salt thereof once a week. In some embodiments, the method comprises administering to the patient about 80 mg to about 150 mg of the compound of Formula (I) or pharmaceutically acceptable salt thereof once a week. In some embodiments, the method comprises administering to the patient about 80 mg to about 100 mg of the compound of Formula (I) or pharmaceutically acceptable salt thereof once a week. In some embodiments, the method comprises administering to the patient about 90 mg to about 200 mg of the compound of Formula (I) or pharmaceutically acceptable salt thereof once a week. In some embodiments, the method comprises administering to the patient about 90 mg to about 150 mg of the compound of Formula (I) or pharmaceutically acceptable salt thereof once a week. In some embodiments, the method comprises administering to the patient about 90 mg to about 100 mg of the compound of Formula (I) or pharmaceutically acceptable salt thereof once a week. In some embodiments, the method comprises administering to the patient about 100 mg to about 200 mg of the compound of Formula (I) or pharmaceutically acceptable salt thereof once a week. In some embodiments, the method comprises administering to the patient about 100 mg to about 150 mg of the compound of Formula (I) or pharmaceutically acceptable salt thereof once a week.

In some embodiments, the method comprises administering to the patient about 50 mg of the compound of Formula (I) or pharmaceutically acceptable salt thereof once a week. In some embodiments, the method comprises administering to the patient about 55 mg of the compound of Formula (I) or pharmaceutically acceptable salt thereof once a week. In some embodiments, the method comprises administering to the patient about 60 mg of the compound of Formula (I) or pharmaceutically acceptable salt thereof once a week. In some embodiments, the method comprises administering to the patient about 65 mg of the compound of Formula (I) or pharmaceutically acceptable salt thereof once a week. In some embodiments, the method comprises administering to the patient about 70 mg of the compound of Formula (I) or pharmaceutically acceptable salt thereof once a week. In some embodiments, the method comprises administering to the patient about 75 mg of the compound of Formula (I) or pharmaceutically acceptable salt thereof once a week. In some embodiments, the method comprises administering to the patient about 80 mg of the compound of Formula (I) or pharmaceutically acceptable salt thereof once a week. In some embodiments, the method comprises administering to the patient about 85 mg of the compound of Formula (I) or pharmaceutically acceptable salt thereof once a week. In some embodiments, the method comprises administering to the patient about 90 mg of the compound of Formula (I) or pharmaceutically acceptable salt thereof once a week. In some embodiments, the method comprises administering to the patient about 95 mg of the compound of Formula (I) or pharmaceutically acceptable salt thereof once a week. In some embodiments, the method comprises administering to the patient about 100 mg of the compound of Formula (I) or pharmaceutically acceptable salt thereof once a week. In some embodiments, the method comprises administering to the patient about 105 mg of the compound of Formula (I) or pharmaceutically acceptable salt thereof once a week. In some embodiments, the method comprises administering to the patient about 110 mg of the compound of Formula (I) or pharmaceutically acceptable salt thereof once a week. In some embodiments, the method comprises administering to the patient about 115 mg of the compound of Formula (I) or pharmaceutically acceptable salt thereof once a week. In some embodiments, the method comprises administering to the patient about 120 mg of the compound of Formula (I) or pharmaceutically acceptable salt thereof once a week. In some embodiments, the method comprises administering to the patient about 125 mg of the compound of Formula (I) or pharmaceutically acceptable salt thereof once a week. In some embodiments, the method comprises administering to the patient about 130 mg of the compound of Formula (I) or pharmaceutically acceptable salt thereof once a week. In some embodiments, the method comprises administering to the patient about 135 mg of the compound of Formula (I) or pharmaceutically acceptable salt thereof once a week. In some embodiments, the method comprises administering to the patient about 140 mg of the compound of Formula (I) or pharmaceutically acceptable salt thereof once a week. In some embodiments, the method comprises administering to the patient about 145 mg of the compound of Formula (I) or pharmaceutically acceptable salt thereof once a week. In some embodiments, the method comprises administering to the patient about 150 mg of the compound of Formula (I) or pharmaceutically acceptable salt thereof once a week. In some embodiments, the method comprises administering to the patient about 155 mg of the compound of Formula (I) or pharmaceutically acceptable salt thereof once a week. In some embodiments, the method comprises administering to the patient about 160 mg of the compound of Formula (I) or pharmaceutically acceptable salt thereof once a week. In some embodiments, the method comprises administering to the patient about 165 mg of the compound of Formula (I) or pharmaceutically acceptable salt thereof once a week. In some embodiments, the method comprises administering to the patient about 170 mg of the compound of Formula (I) or pharmaceutically acceptable salt thereof once a week. In some embodiments, the method comprises administering to the patient about 175 mg of the compound of Formula (I) or pharmaceutically acceptable salt thereof once a week. In some embodiments, the method comprises administering to the patient about 180 mg of the compound of Formula (I) or pharmaceutically acceptable salt thereof once a week. In some embodiments, the method comprises administering to the patient about 185 mg of the compound of Formula (I) or pharmaceutically acceptable salt thereof once a week. In some embodiments, the method comprises administering to the patient about 190 mg of the compound of Formula (I) or pharmaceutically acceptable salt thereof once a week. In some embodiments, the method comprises administering to the patient about 195 mg of the compound of Formula (I) or pharmaceutically acceptable salt thereof once a week. In some embodiments, the method comprises administering to the patient about 200 mg of the compound of Formula (I) or pharmaceutically acceptable salt thereof once a week.

In some embodiments, the method comprises administering to the patient about 50 mg to about 200 mg of the compound of Formula (I) or pharmaceutically acceptable salt thereof twice a week. In some embodiments, the method comprises administering to the patient about 50 mg to about 150 mg of the compound of Formula (I) or pharmaceutically acceptable salt thereof twice a week. In some embodiments, the method comprises administering to the patient about 50 mg to about 100 mg of the compound of Formula (I) or pharmaceutically acceptable salt thereof twice a week. In some embodiments, the method comprises administering to the patient about 60 mg to about 200 mg of the compound of Formula (I) or pharmaceutically acceptable salt thereof twice a week. In some embodiments, the method comprises administering to the patient about 60 mg to about 150 mg of the compound of Formula (I) or pharmaceutically acceptable salt thereof twice a week. In some embodiments, the method comprises administering to the patient about 60 mg to about 100 mg of the compound of Formula (I) or pharmaceutically acceptable salt thereof twice a week. In some embodiments, the method comprises administering to the patient about 70 mg to about 200 mg of the compound of Formula (I) or pharmaceutically acceptable salt thereof twice a week. In some embodiments, the method comprises administering to the patient about 70 mg to about 150 mg of the compound of Formula (I) or pharmaceutically acceptable salt thereof twice a week. In some embodiments, the method comprises administering to the patient about 70 mg to about 100 mg of the compound of Formula (I) or pharmaceutically acceptable salt thereof twice a week. In some embodiments, the method comprises administering to the patient about 80 mg to about 200 mg of the compound of Formula (I) or pharmaceutically acceptable salt thereof twice a week. In some embodiments, the method comprises administering to the patient about 80 mg to about 150 mg of the compound of Formula (I) or pharmaceutically acceptable salt thereof twice a week. In some embodiments, the method comprises administering to the patient about 80 mg to about 100 mg of the compound of Formula (I) or pharmaceutically acceptable salt thereof twice a week. In some embodiments, the method comprises administering to the patient about 90 mg to about 200 mg of the compound of Formula (I) or pharmaceutically acceptable salt thereof twice a week. In some embodiments, the method comprises administering to the patient about 90 mg to about 150 mg of the compound of Formula (I) or pharmaceutically acceptable salt thereof twice a week. In some embodiments, the method comprises administering to the patient about 90 mg to about 100 mg of the compound of Formula (I) or pharmaceutically acceptable salt thereof twice a week. In some embodiments, the method comprises administering to the patient about 100 mg to about 200 mg of the compound of Formula (I) or pharmaceutically acceptable salt thereof twice a week. In some embodiments, the method comprises administering to the patient about 100 mg to about 150 mg of the compound of Formula (I) or pharmaceutically acceptable salt thereof twice a week.

In some embodiments, the method comprises administering to the patient about 50 mg of the compound of Formula (I) or pharmaceutically acceptable salt thereof twice a week. In some embodiments, the method comprises administering to the patient about 55 mg of the compound of Formula (I) or pharmaceutically acceptable salt thereof twice a week. In some embodiments, the method comprises administering to the patient about 60 mg of the compound of Formula (I) or pharmaceutically acceptable salt thereof twice a week. In some embodiments, the method comprises administering to the patient about 65 mg of the compound of Formula (I) or pharmaceutically acceptable salt thereof twice a week. In some embodiments, the method comprises administering to the patient about 70 mg of the compound of Formula (I) or pharmaceutically acceptable salt thereof twice a week. In some embodiments, the method comprises administering to the patient about 75 mg of the compound of Formula (I) or pharmaceutically acceptable salt thereof twice a week. In some embodiments, the method comprises administering to the patient about 80 mg of the compound of Formula (I) or pharmaceutically acceptable salt thereof twice a week. In some embodiments, the method comprises administering to the patient about 85 mg of the compound of Formula (I) or pharmaceutically acceptable salt thereof twice a week. In some embodiments, the method comprises administering to the patient about 90 mg of the compound of Formula (I) or pharmaceutically acceptable salt thereof twice a week. In some embodiments, the method comprises administering to the patient about 95 mg of the compound of Formula (I) or pharmaceutically acceptable salt thereof twice a week. In some embodiments, the method comprises administering to the patient about 100 mg of the compound of Formula (I) or pharmaceutically acceptable salt thereof twice a week. In some embodiments, the method comprises administering to the patient about 105 mg of the compound of Formula (I) or pharmaceutically acceptable salt thereof twice a week. In some embodiments, the method comprises administering to the patient about 110 mg of the compound of Formula (I) or pharmaceutically acceptable salt thereof twice a week. In some embodiments, the method comprises administering to the patient about 115 mg of the compound of Formula (I) or pharmaceutically acceptable salt thereof twice a week. In some embodiments, the method comprises administering to the patient about 120 mg of the compound of Formula (I) or pharmaceutically acceptable salt thereof twice a week. In some embodiments, the method comprises administering to the patient about 125 mg of the compound of Formula (I) or pharmaceutically acceptable salt thereof twice a week. In some embodiments, the method comprises administering to the patient about 130 mg of the compound of Formula (I) or pharmaceutically acceptable salt thereof twice a week. In some embodiments, the method comprises administering to the patient about 135 mg of the compound of Formula (I) or pharmaceutically acceptable salt thereof twice a week. In some embodiments, the method comprises administering to the patient about 140 mg of the compound of Formula (I) or pharmaceutically acceptable salt thereof twice a week. In some embodiments, the method comprises administering to the patient about 145 mg of the compound of Formula (I) or pharmaceutically acceptable salt thereof twice a week. In some embodiments, the method comprises administering to the patient about 150 mg of the compound of Formula (I) or pharmaceutically acceptable salt thereof twice a week. In some embodiments, the method comprises administering to the patient about 155 mg of the compound of Formula (I) or pharmaceutically acceptable salt thereof twice a week. In some embodiments, the method comprises administering to the patient about 160 mg of the compound of Formula (I) or pharmaceutically acceptable salt thereof twice a week. In some embodiments, the method comprises administering to the patient about 165 mg of the compound of Formula (I) or pharmaceutically acceptable salt thereof twice a week. In some embodiments, the method comprises administering to the patient about 170 mg of the compound of Formula (I) or pharmaceutically acceptable salt thereof twice a week. In some embodiments, the method comprises administering to the patient about 175 mg of the compound of Formula (I) or pharmaceutically acceptable salt thereof twice a week. In some embodiments, the method comprises administering to the patient about 180 mg of the compound of Formula (I) or pharmaceutically acceptable salt thereof twice a week. In some embodiments, the method comprises administering to the patient about 185 mg of the compound of Formula (I) or pharmaceutically acceptable salt thereof twice a week. In some embodiments, the method comprises administering to the patient about 190 mg of the compound of Formula (I) or pharmaceutically acceptable salt thereof twice a week. In some embodiments, the method comprises administering to the patient about 195 mg of the compound of Formula (I) or pharmaceutically acceptable salt thereof twice a week. In some embodiments, the method comprises administering to the patient about 200 mg of the compound of Formula (I) or pharmaceutically acceptable salt thereof twice a week.

In some embodiments, the method comprises administering to the patient about 50 mg to about 200 mg of the compound of Formula (I) or pharmaceutically acceptable salt thereof three times a week. In some embodiments, the method comprises administering to the patient about 50 mg to about 150 mg of the compound of Formula (I) or pharmaceutically acceptable salt thereof three times a week. In some embodiments, the method comprises administering to the patient about 50 mg to about 100 mg of the compound of Formula (I) or pharmaceutically acceptable salt thereof three times a week. In some embodiments, the method comprises administering to the patient about 60 mg to about 200 mg of the compound of Formula (I) or pharmaceutically acceptable salt thereof three times a week. In some embodiments, the method comprises administering to the patient about 60 mg to about 150 mg of the compound of Formula (I) or pharmaceutically acceptable salt thereof three times a week. In some embodiments, the method comprises administering to the patient about 60 mg to about 100 mg of the compound of Formula (I) or pharmaceutically acceptable salt thereof three times a week. In some embodiments, the method comprises administering to the patient about 70 mg to about 200 mg of the compound of Formula (I) or pharmaceutically acceptable salt thereof three times a week. In some embodiments, the method comprises administering to the patient about 70 mg to about 150 mg of the compound of Formula (I) or pharmaceutically acceptable salt thereof three times a week. In some embodiments, the method comprises administering to the patient about 70 mg to about 100 mg of the compound of Formula (I) or pharmaceutically acceptable salt thereof three times a week. In some embodiments, the method comprises administering to the patient about 80 mg to about 200 mg of the compound of Formula (I) or pharmaceutically acceptable salt thereof three times a week. In some embodiments, the method comprises administering to the patient about 80 mg to about 150 mg of the compound of Formula (I) or pharmaceutically acceptable salt thereof three times a week. In some embodiments, the method comprises administering to the patient about 80 mg to about 100 mg of the compound of Formula (I) or pharmaceutically acceptable salt thereof three times a week. In some embodiments, the method comprises administering to the patient about 90 mg to about 200 mg of the compound of Formula (I) or pharmaceutically acceptable salt thereof three times a week. In some embodiments, the method comprises administering to the patient about 90 mg to about 150 mg of the compound of Formula (I) or pharmaceutically acceptable salt thereof three times a week. In some embodiments, the method comprises administering to the patient about 90 mg to about 100 mg of the compound of Formula (I) or pharmaceutically acceptable salt thereof three times a week. In some embodiments, the method comprises administering to the patient about 100 mg to about 200 mg of the compound of Formula (I) or pharmaceutically acceptable salt thereof three times a week. In some embodiments, the method comprises administering to the patient about 100 mg to about 150 mg of the compound of Formula (I) or pharmaceutically acceptable salt thereof three times a week.

In some embodiments, the method comprises administering to the patient about 50 mg of the compound of Formula (I) or pharmaceutically acceptable salt thereof three times a week. In some embodiments, the method comprises administering to the patient about 55 mg of the compound of Formula (I) or pharmaceutically acceptable salt thereof three times a week. In some embodiments, the method comprises administering to the patient about 60 mg of the compound of Formula (I) or pharmaceutically acceptable salt thereof three times a week. In some embodiments, the method comprises administering to the patient about 65 mg of the compound of Formula (I) or pharmaceutically acceptable salt thereof three times a week. In some embodiments, the method comprises administering to the patient about 70 mg of the compound of Formula (I) or pharmaceutically acceptable salt thereof three times a week. In some embodiments, the method comprises administering to the patient about 75 mg of the compound of Formula (I) or pharmaceutically acceptable salt thereof three times a week. In some embodiments, the method comprises administering to the patient about 80 mg of the compound of Formula (I) or pharmaceutically acceptable salt thereof three times a week. In some embodiments, the method comprises administering to the patient about 85 mg of the compound of Formula (I) or pharmaceutically acceptable salt thereof three times a week. In some embodiments, the method comprises administering to the patient about 90 mg of the compound of Formula (I) or pharmaceutically acceptable salt thereof three times a week. In some embodiments, the method comprises administering to the patient about 95 mg of the compound of Formula (I) or pharmaceutically acceptable salt thereof three times a week. In some embodiments, the method comprises administering to the patient about 100 mg of the compound of Formula (I) or pharmaceutically acceptable salt thereof three times a week. In some embodiments, the method comprises administering to the patient about 105 mg of the compound of Formula (I) or pharmaceutically acceptable salt thereof three times a week. In some embodiments, the method comprises administering to the patient about 110 mg of the compound of Formula (I) or pharmaceutically acceptable salt thereof three times a week. In some embodiments, the method comprises administering to the patient about 115 mg of the compound of Formula (I) or pharmaceutically acceptable salt thereof three times a week. In some embodiments, the method comprises administering to the patient about 120 mg of the compound of Formula (I) or pharmaceutically acceptable salt thereof three times a week. In some embodiments, the method comprises administering to the patient about 125 mg of the compound of Formula (I) or pharmaceutically acceptable salt thereof three times a week. In some embodiments, the method comprises administering to the patient about 130 mg of the compound of Formula (I) or pharmaceutically acceptable salt thereof three times a week. In some embodiments, the method comprises administering to the patient about 135 mg of the compound of Formula (I) or pharmaceutically acceptable salt thereof three times a week. In some embodiments, the method comprises administering to the patient about 140 mg of the compound of Formula (I) or pharmaceutically acceptable salt thereof three times a week. In some embodiments, the method comprises administering to the patient about 145 mg of the compound of Formula (I) or pharmaceutically acceptable salt thereof three times a week. In some embodiments, the method comprises administering to the patient about 150 mg of the compound of Formula (I) or pharmaceutically acceptable salt thereof three times a week. In some embodiments, the method comprises administering to the patient about 155 mg of the compound of Formula (I) or pharmaceutically acceptable salt thereof three times a week. In some embodiments, the method comprises administering to the patient about 160 mg of the compound of Formula (I) or pharmaceutically acceptable salt thereof three times a week. In some embodiments, the method comprises administering to the patient about 165 mg of the compound of Formula (I) or pharmaceutically acceptable salt thereof three times a week. In some embodiments, the method comprises administering to the patient about 170 mg of the compound of Formula (I) or pharmaceutically acceptable salt thereof three times a week. In some embodiments, the method comprises administering to the patient about 175 mg of the compound of Formula (I) or pharmaceutically acceptable salt thereof three times a week. In some embodiments, the method comprises administering to the patient about 180 mg of the compound of Formula (I) or pharmaceutically acceptable salt thereof three times a week. In some embodiments, the method comprises administering to the patient about 185 mg of the compound of Formula (I) or pharmaceutically acceptable salt thereof three times a week. In some embodiments, the method comprises administering to the patient about 190 mg of the compound of Formula (I) or pharmaceutically acceptable salt thereof three times a week. In some embodiments, the method comprises administering to the patient about 195 mg of the compound of Formula (I) or pharmaceutically acceptable salt thereof three times a week. In some embodiments, the method comprises administering to the patient about 200 mg of the compound of Formula (I) or pharmaceutically acceptable salt thereof three times a week.

In some embodiments, the method comprises administering to the patient about 50 mg to about 200 mg of the compound of Formula (I) or pharmaceutically acceptable salt thereof every other day. In some embodiments, the method comprises administering to the patient about 50 mg to about 150 mg of the compound of Formula (I) or pharmaceutically acceptable salt thereof every other day. In some embodiments, the method comprises administering to the patient about 50 mg to about 100 mg of the compound of Formula (I) or pharmaceutically acceptable salt thereof every other day. In some embodiments, the method comprises administering to the patient about 60 mg to about 200 mg of the compound of Formula (I) or pharmaceutically acceptable salt thereof every other day. In some embodiments, the method comprises administering to the patient about 60 mg to about 150 mg of the compound of Formula (I) or pharmaceutically acceptable salt thereof every other day. In some embodiments, the method comprises administering to the patient about 60 mg to about 100 mg of the compound of Formula (I) or pharmaceutically acceptable salt thereof every other day. In some embodiments, the method comprises administering to the patient about 70 mg to about 200 mg of the compound of Formula (I) or pharmaceutically acceptable salt thereof every other day. In some embodiments, the method comprises administering to the patient about 70 mg to about 150 mg of the compound of Formula (I) or pharmaceutically acceptable salt thereof every other day. In some embodiments, the method comprises administering to the patient about 70 mg to about 100 mg of the compound of Formula (I) or pharmaceutically acceptable salt thereof every other day. In some embodiments, the method comprises administering to the patient about 80 mg to about 200 mg of the compound of Formula (I) or pharmaceutically acceptable salt thereof every other day. In some embodiments, the method comprises administering to the patient about 80 mg to about 150 mg of the compound of Formula (I) or pharmaceutically acceptable salt thereof every other day. In some embodiments, the method comprises administering to the patient about 80 mg to about 100 mg of the compound of Formula (I) or pharmaceutically acceptable salt thereof every other day. In some embodiments, the method comprises administering to the patient about 90 mg to about 200 mg of the compound of Formula (I) or pharmaceutically acceptable salt thereof every other day. In some embodiments, the method comprises administering to the patient about 90 mg to about 150 mg of the compound of Formula (I) or pharmaceutically acceptable salt thereof every other day. In some embodiments, the method comprises administering to the patient about 90 mg to about 100 mg of the compound of Formula (I) or pharmaceutically acceptable salt thereof every other day. In some embodiments, the method comprises administering to the patient about 100 mg to about 200 mg of the compound of Formula (I) or pharmaceutically acceptable salt thereof every other day. In some embodiments, the method comprises administering to the patient about 100 mg to about 150 mg of the compound of Formula (I) or pharmaceutically acceptable salt thereof every other day.

In some embodiments, the method comprises administering to the patient about 50 mg of the compound of Formula (I) or pharmaceutically acceptable salt thereof every other day. In some embodiments, the method comprises administering to the patient about 55 mg of the compound of Formula (I) or pharmaceutically acceptable salt thereof every other day. In some embodiments, the method comprises administering to the patient about 60 mg of the compound of Formula (I) or pharmaceutically acceptable salt thereof every other day. In some embodiments, the method comprises administering to the patient about 65 mg of the compound of Formula (I) or pharmaceutically acceptable salt thereof every other day. In some embodiments, the method comprises administering to the patient about 70 mg of the compound of Formula (I) or pharmaceutically acceptable salt thereof every other day. In some embodiments, the method comprises administering to the patient about 75 mg of the compound of Formula (I) or pharmaceutically acceptable salt thereof every other day. In some embodiments, the method comprises administering to the patient about 80 mg of the compound of Formula (I) or pharmaceutically acceptable salt thereof every other day. In some embodiments, the method comprises administering to the patient about 85 mg of the compound of Formula (I) or pharmaceutically acceptable salt thereof every other day. In some embodiments, the method comprises administering to the patient about 90 mg of the compound of Formula (I) or pharmaceutically acceptable salt thereof every other day. In some embodiments, the method comprises administering to the patient about 95 mg of the compound of Formula (I) or pharmaceutically acceptable salt thereof every other day. In some embodiments, the method comprises administering to the patient about 100 mg of the compound of Formula (I) or pharmaceutically acceptable salt thereof every other day. In some embodiments, the method comprises administering to the patient about 105 mg of the compound embodiments, the method comprises administering to the patient about 110 mg of the compound of Formula (I) or pharmaceutically acceptable salt thereof every other day. In some embodiments, the method comprises administering to the patient about 115 mg of the compound of Formula (I) or pharmaceutically acceptable salt thereof every other day. In some embodiments, the method comprises administering to the patient about 120 mg of the compound of Formula (I) or pharmaceutically acceptable salt thereof every other day. In some embodiments, the method comprises administering to the patient about 125 mg of the compound of Formula (I) or pharmaceutically acceptable salt thereof every other day. In some embodiments, the method comprises administering to the patient about 130 mg of the compound of Formula (I) or pharmaceutically acceptable salt thereof every other day. In some embodiments, the method comprises administering to the patient about 135 mg of the compound of Formula (I) or pharmaceutically acceptable salt thereof every other day. In some embodiments, the method comprises administering to the patient about 140 mg of the compound of Formula (I) or pharmaceutically acceptable salt thereof every other day. In some embodiments, the method comprises administering to the patient about 145 mg of the compound of Formula (I) or pharmaceutically acceptable salt thereof every other day. In some embodiments, the method comprises administering to the patient about 150 mg of the compound of Formula (I) or pharmaceutically acceptable salt thereof every other day. In some embodiments, the method comprises administering to the patient about 155 mg of the compound of Formula (I) or pharmaceutically acceptable salt thereof every other day. In some embodiments, the method comprises administering to the patient about 160 mg of the compound of Formula (I) or pharmaceutically acceptable salt thereof every other day. In some embodiments, the method comprises administering to the patient about 165 mg of the compound of Formula (I) or pharmaceutically acceptable salt thereof every other day. In some embodiments, the method comprises administering to the patient about 170 mg of the compound of Formula (I) or pharmaceutically acceptable salt thereof every other day. In some embodiments, the method comprises administering to the patient about 175 mg of the compound of Formula (I) or pharmaceutically acceptable salt thereof every other day. In some embodiments, the method comprises administering to the patient about 180 mg of the compound of Formula (I) or pharmaceutically acceptable salt thereof every other day. In some embodiments, the method comprises administering to the patient about 185 mg of the compound of Formula (I) or pharmaceutically acceptable salt thereof every other day. In some embodiments, the method comprises administering to the patient about 190 mg of the compound of Formula (I) or pharmaceutically acceptable salt thereof every other day. In some embodiments, the method comprises administering to the patient about 195 mg of the compound of Formula (I) or pharmaceutically acceptable salt thereof every other day. In some embodiments, the method comprises administering to the patient about 200 mg of the compound of Formula (I) or pharmaceutically acceptable salt thereof every other day.

In some embodiments, the method further comprises administering a combination agent to the patient in need thereof. In some embodiments, the combination agent is carboplatin. In some embodiments, the combination agent is paclitaxel. In some embodiments, the method further comprises administering pemetrexed to the patient in need thereof. In some embodiments, the combination agent is intravenously administered to the patient in need thereof. In some embodiments, the method further comprises administering one or more combination agents to the patient in need thereof. In some embodiments, the method further comprises administering carboplatin and paclitaxel.

In some embodiments, the patient has previously been administered at least one prior line of therapy. In some embodiments, the at least one prior line of therapy is a platinum-based therapy. In some embodiments, the patient has previously been administered carboplatin and/or pemetrexed. In some embodiments, the patient has previously been administered cisplatin and/or pemetrexed.

The one or more combination agents administered to a patient in the methods of treating pancreatic ductal adenocarcinoma described herein can be dosed to achieve various concentrations of the combination agents in the patient's plasma, which can be quantified by the area under the curve (AUC) for each combination agent administered, e.g., intravenously administered. In some embodiments, the combination agent (e.g., carboplatin) is intravenously administered to the patient in an amount sufficient to provide an AUC of about 5 to about 7 (e.g., an AUC of about 5 or about 6).

In some embodiments, the paclitaxel is intravenously administered to the patient, e.g., by intravenous infusion. The intravenously administered paclitaxel can be administered to a patient at various dose levels. In some embodiments, the paclitaxel is administered to the patient at about 50 mg/m² to about 150 mg/m². In some embodiments, the paclitaxel is administered to the patient at about 50 mg/m² to about 100 mg/m². In some embodiments, the paclitaxel is administered to the patient at about 60 mg/m² to about 80 mg/m². In some embodiments, the paclitaxel is administered to the patient at about 60 mg/m². In some embodiments, the paclitaxel is administered to the patient at about 80 mg/m². In some embodiments, the paclitaxel is administered to the patient at about 135 mg/m². In some embodiments, the paclitaxel is administered to the patient at about 175 mg/m².

Furthermore, the paclitaxel can be administered to the patient at various frequencies. In some embodiments, the paclitaxel is administered to the patient in need thereof once per week (e.g., at about 60 mg/m² once per week or about 80 mg/m² once per week). In some embodiments, the paclitaxel is administered to the patient in need thereof once every three weeks (e.g., at about 135 mg/m² once every three weeks or about 175 mg/m² once every three weeks). In some embodiments, the paclitaxel is administered to the patient in need thereof. On Day 1, Day 8 and Day 15 of repeated 28-day cycles. In some embodiments, the paclitaxel is administered to the patient by intravenous infusion once per week. In some embodiments, the paclitaxel is administered to the patient by intravenous infusion once per week for three consecutive weeks. In some embodiments, the method comprises administering paclitaxel to the patient by intravenous infusion for more than one cycle, wherein each cycle comprises administering paclitaxel to the patient by intravenous infusion once per week for three consecutive weeks (e.g., once per week for three consecutive weeks at about 60 mg/m² or about 80 mg/m²). In some embodiments, the method comprises not administering paclitaxel to the patient for at least one week between each cycle.

In some embodiments, the paclitaxel is administered to the patient by infusion once every three weeks. In some embodiments, the paclitaxel is administered to the patient for about 60 minutes once per week. In some embodiments, the paclitaxel is administered to the patient for about 3 hours once per week. In some embodiments, the paclitaxel is administered to the patient for about 60 minutes once every three weeks. In some embodiments, the paclitaxel is administered to the patient for about 3 hours once every three weeks. In some embodiments, the paclitaxel is administered to the patient by intravenous infusion for about 60 minutes once per week. In some embodiments, the paclitaxel is administered to the patient by infusion for about 3 hours once per week. In some embodiments, the paclitaxel is administered to the patient by intravenous infusion for about 60 minutes once every three weeks. In some embodiments, the paclitaxel is administered to the patient by infusion for about 3 hours once every three weeks. In some embodiments, the paclitaxel is continuously administered to the patient for about 60 minutes once per week. In some embodiments, the paclitaxel is continuously administered to the patient for about 3 hours once per week. In some embodiments, the paclitaxel is continuously administered to the patient for about 60 minutes once every three weeks. In some embodiments, the paclitaxel is continuously administered to the patient for about 3 hours once every three weeks. In some embodiments, the paclitaxel is continuously administered to the patient by infusion for about 60 minutes once per week. In some embodiments, the paclitaxel is continuously administered to the patient by intravenous infusion for about 3 hours once per week. In some embodiments, the paclitaxel is continuously administered to the patient by infusion for about 60 minutes once every three weeks. In some embodiments, the paclitaxel is continuously administered to the patient by intravenous infusion for about 3 hours once every three weeks. In some embodiments, the paclitaxel is administered for about 60 minutes to the patient in need thereof on Day 1, Day 8 and Day 15 of repeated 28-day cycles. In some embodiments, the paclitaxel is administered by intravenous infusion to the patient in need thereof on Day 1, Day 8 and Day 15 of repeated 28-day cycles. In some embodiments, the paclitaxel is administered by intravenous infusion for about 60 minutes to the patient in need thereof on Day 1, Day 8 and Day 15 of repeated 28-day cycles. In some embodiments, the paclitaxel is administered by intravenous infusion at about 60 mg/m² for about 60 minutes to the patient in need thereof on Day 1, Day 8 and Day 15 of repeated 28-day cycles. In some embodiments, the paclitaxel is administered by intravenous infusion at about 70 mg/m² for about 60 minutes to the patient in need thereof on Day 1, Day 8 and Day 15 of repeated 28-day cycles. In some embodiments, the paclitaxel is administered by intravenous infusion at about 80 mg/m² for about 60 minutes to the patient in need thereof on Day 1, Day 8 and Day 15 of repeated 28-day cycles. In some embodiments, the paclitaxel is administered by intravenous infusion at about 85 mg/m² for about 60 minutes to the patient in need thereof on Day 1, Day 8 and Day 15 of repeated 28-day cycles. In some embodiments, the paclitaxel is administered by intravenous infusion at about 90 mg/m² for about 60 minutes to the patient in need thereof on Day 1, Day 8 and Day 15 of repeated 28-day cycles. In some embodiments, the paclitaxel is administered by intravenous infusion at about 100 mg/m² for about 60 minutes to the patient in need thereof on Day 1, Day 8 and Day 15 of repeated 28-day cycles. In some embodiments, the paclitaxel is administered by intravenous infusion at about 110 mg/m² for about 60 minutes to the patient in need thereof on Day 1, Day 8 and Day 15 of repeated 28-day cycles. In some embodiments, the paclitaxel is administered by intravenous infusion at about 120 mg/m² for about 60 minutes to the patient in need thereof on Day 1, Day 8 and Day 15 of repeated 28-day cycles. In some embodiments, the paclitaxel is administered by intravenous infusion at about 130 mg/m² for about 60 minutes to the patient in need thereof on Day 1, Day 8 and Day 15 of repeated 28-day cycles. In some embodiments, the paclitaxel is administered by intravenous infusion at about 135 mg/m² for about 60 minutes to the patient in need thereof on Day 1, Day 8 and Day 15 of repeated 28-day cycles. In some embodiments, the paclitaxel is administered by intravenous infusion at about 140 mg/m² for about 60 minutes to the patient in need thereof on Day 1, Day 8 and Day 15 of repeated 28-day cycles. In some embodiments, the paclitaxel is administered by intravenous infusion at about 145 mg/m² for about 60 minutes to the patient in need thereof on Day 1, Day 8 and Day 15 of repeated 28-day cycles. In some embodiments, the paclitaxel is administered by intravenous infusion at about 150 mg/m² for about 60 minutes to the patient in need thereof on Day 1, Day 8 and Day 15 of repeated 28-day cycles. In some embodiments, the paclitaxel is administered by intravenous infusion at about 155 mg/m² for about 60 minutes to the patient in need thereof on Day 1, Day 8 and Day 15 of repeated 28-day cycles. In some embodiments, the paclitaxel is administered by intravenous infusion at about 160 mg/m² for about 60 minutes to the patient in need thereof on Day 1, Day 8 and Day 15 of repeated 28-day cycles. In some embodiments, the paclitaxel is administered by intravenous infusion at about 165 mg/m² for about 60 minutes to the patient in need thereof on Day 1, Day 8 and Day 15 of repeated 28-day cycles. In some embodiments, the paclitaxel is administered by intravenous infusion at about 170 mg/m² for about 60 minutes to the patient in need thereof on Day 1, Day 8 and Day 15 of repeated 28-day cycles. In some embodiments, the paclitaxel is administered by intravenous infusion at about 175 mg/m² for about 60 minutes to the patient in need thereof on Day 1, Day 8 and Day 15 of repeated 28-day cycles. In some embodiments, the paclitaxel is administered by intravenous infusion at about 180 mg/m² for about 60 minutes to the patient in need thereof on Day 1, Day 8 and Day 15 of repeated 28-day cycles.

A method of treating pancreatic ductal adenocarcinoma described herein can further comprise reducing each of the amount of the compound of Formula (I) or pharmaceutically acceptable salt thereof and the combination agent administered to the patient after one or more months of administering said compound of Formula (I) or pharmaceutically acceptable salt thereof and said combination agent to the patient in need thereof.

In embodiments of the methods of treating an pancreatic ductal adenocarcinoma described herein, the disclosure also contemplates administration of the compound of Formula (I) or a pharmaceutically acceptable salt to a patient in need thereof prior to (neo-adjuvant) or after (adjuvant) surgery (e.g., surgical treatment of a solid tumor). In some embodiments, the compound of Formula (I) or a pharmaceutically acceptable salt is administered as an adjuvant. In some embodiments, the compound of Formula (I) or a pharmaceutically acceptable salt is administered as a neo-adjuvant. In some embodiments, the compound of Formula (I) or a pharmaceutically acceptable salt is administered as a neo-adjuvant and an adjuvant. In some embodiments, the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 100 years. In some embodiments, administration of the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 5 years. In some embodiments, administration of the compound of Formula (I) or a pharmaceutically acceptable salt is administered to a patient in need thereof as a neo-adjuvant for a period of 3 months to 6 months, followed by administration of the compound of Formula (I) or a pharmaceutically acceptable salt as an adjuvant for a period from 1 day to 5 years. In some embodiments, no administration of the compound of Formula (I) or a pharmaceutically acceptable salt occurs prior to surgery. In some embodiments, the compound of Formula (I) or a pharmaceutically acceptable salt is administered as an adjuvant for a period from 1 day to 100 years. In some embodiments, the compound of Formula (I) or a pharmaceutically acceptable salt is administered as an adjuvant for a period from 1 day to 5 years.

In another embodiment, provided herein is a method for achieving at least a partial response in a patient having pancreatic ductal adenocarcinoma, comprising administering to the patient an effective amount of the compound of Formula (I)

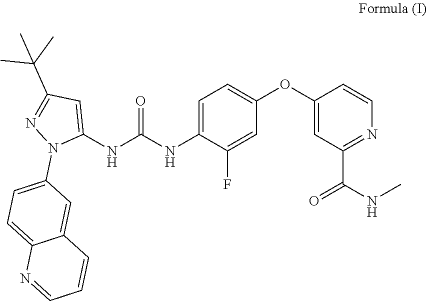 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In some embodiments, the methods further comprise administering a combination agent to the patient in need thereof. In some embodiments, the combination agent is carboplatin. In some embodiments, the combination agent is paclitaxel. In some embodiments, the method further comprises administering pemetrexed to the patient in need thereof. In some embodiments, the method further comprises administering one or more combination agents to the patient in need thereof. In some embodiments, the method further comprises administering carboplatin and paclitaxel.

In some embodiments, the patient has previously been administered at least one prior line of therapy. In some embodiments, the therapy is a platinum-based therapy. In some embodiments, the patient previously been administered carboplatin and/or pemetrexed. In some embodiments, the patient previously been administered cisplatin and/or pemetrexed.

In embodiments of the methods for achieving at least a partial response in a patient having pancreatic ductal adenocarcinoma described herein, the compound of Formula (I) or pharmaceutically acceptable salt thereof can be orally administered to the patient in need thereof. In certain embodiments, the compound of Formula (I) or pharmaceutically acceptable salt thereof is administered to the patient daily. In some embodiments, the method comprises administering to the patient about 50 mg to about 200 mg of the compound of Formula (I) or pharmaceutically acceptable salt thereof per day. In some embodiments, the method comprises administering to the patient about 50 mg to about 150 mg of the compound of Formula (I) or pharmaceutically acceptable salt thereof per day. In some embodiments, the method comprises administering to the patient about 50 mg to about 100 mg of the compound of Formula (I) or pharmaceutically acceptable salt thereof per day. In some embodiments, the method comprises administering to the patient about 60 mg to about 200 mg of the compound of Formula (I) or pharmaceutically acceptable salt thereof per day. In some embodiments, the method comprises administering to the patient about 60 mg to about 150 mg of the compound of Formula (I) or pharmaceutically acceptable salt thereof per day. In some embodiments, the method comprises administering to the patient about 60 mg to about 100 mg of the compound of Formula (I) or pharmaceutically acceptable salt thereof per day. In some embodiments, the method comprises administering to the patient about 70 mg to about 200 mg of the compound of Formula (I) or pharmaceutically acceptable salt thereof per day. In some embodiments, the method comprises administering to the patient about 70 mg to about 150 mg of the compound of Formula (I) or pharmaceutically acceptable salt thereof per day. In some embodiments, the method comprises administering to the patient about 70 mg to about 100 mg of the compound of Formula (I) or pharmaceutically acceptable salt thereof per day. In some embodiments, the method comprises administering to the patient about 80 mg to about 200 mg of the compound of Formula (I) or pharmaceutically acceptable salt thereof per day. In some embodiments, the method comprises administering to the patient about 80 mg to about 150 mg of the compound of Formula (I) or pharmaceutically acceptable salt thereof per day. In some embodiments, the method comprises administering to the patient about 80 mg to about 100 mg of the compound of Formula (I) or pharmaceutically acceptable salt thereof per day. In some embodiments, the method comprises administering to the patient about 90 mg to about 200 mg of the compound of Formula (I) or pharmaceutically acceptable salt thereof per day. In some embodiments, the method comprises administering to the patient about 90 mg to about 150 mg of the compound of Formula (I) or pharmaceutically acceptable salt thereof per day. In some embodiments, the method comprises administering to the patient about 90 mg to about 100 mg of the compound of Formula (I) or pharmaceutically acceptable salt thereof per day. In some embodiments, the method comprises administering to the patient about 100 mg to about 200 mg of the compound of Formula (I) or pharmaceutically acceptable salt thereof per day. In some embodiments, the method comprises administering to the patient about 100 mg to about 150 mg of the compound of Formula (I) or pharmaceutically acceptable salt thereof per day.

In some embodiments, the method comprises administering to the patient about 50 mg of the compound of Formula (I) or pharmaceutically acceptable salt thereof per day. In some embodiments, the method comprises administering to the patient about 55 mg of the compound of Formula (I) or pharmaceutically acceptable salt thereof per day. In some embodiments, the method comprises administering to the patient about 60 mg of the compound of Formula (I) or pharmaceutically acceptable salt thereof per day. In some embodiments, the method comprises administering to the patient about 65 mg of the compound of Formula (I) or pharmaceutically acceptable salt thereof per day. In some embodiments, the method comprises administering to the patient about 70 mg of the compound of Formula (I) or pharmaceutically acceptable salt thereof per day. In some embodiments, the method comprises administering to the patient about 75 mg of the compound of Formula (I) or pharmaceutically acceptable salt thereof per day. In some embodiments, the method comprises administering to the patient about 80 mg of the compound of Formula (I) or pharmaceutically acceptable salt thereof per day. In some embodiments, the method comprises administering to the patient about 85 mg of the compound of Formula (I) or pharmaceutically acceptable salt thereof per day. In some embodiments, the method comprises administering to the patient about 90 mg of the compound of Formula (I) or pharmaceutically acceptable salt thereof per day. In some embodiments, the method comprises administering to the patient about 95 mg of the compound of Formula (I) or pharmaceutically acceptable salt thereof per day. In some embodiments, the method comprises administering to the patient about 100 mg of the compound of Formula (I) or pharmaceutically acceptable salt thereof per day. In some embodiments, the method comprises administering to the patient about 105 mg of the compound of Formula (I) or pharmaceutically acceptable salt thereof per day. In some embodiments, the method comprises administering to the patient about 110 mg of the compound of Formula (I) or pharmaceutically acceptable salt thereof per day. In some embodiments, the method comprises administering to the patient about 115 mg of the compound of Formula (I) or pharmaceutically acceptable salt thereof per day. In some embodiments, the method comprises administering to the patient about 120 mg of the compound of Formula (I) or pharmaceutically acceptable salt thereof per day. In some embodiments, the method comprises administering to the patient about 125 mg of the compound of Formula (I) or pharmaceutically acceptable salt thereof per day. In some embodiments, the method comprises administering to the patient about 130 mg of the compound of Formula (I) or pharmaceutically acceptable salt thereof per day. In some embodiments, the method comprises administering to the patient about 135 mg of the compound of Formula (I) or pharmaceutically acceptable salt thereof per day. In some embodiments, the method comprises administering to the patient about 140 mg of the compound of Formula (I) or pharmaceutically acceptable salt thereof per day. In some embodiments, the method comprises administering to the patient about 145 mg of the compound of Formula (I) or pharmaceutically acceptable salt thereof per day. In some embodiments, the method comprises administering to the patient about 150 mg of the compound of Formula (I) or pharmaceutically acceptable salt thereof per day. In some embodiments, the method comprises administering to the patient about 155 mg of the compound of Formula (I) or pharmaceutically acceptable salt thereof per day. In some embodiments, the method comprises administering to the patient about 160 mg of the compound of Formula (I) or pharmaceutically acceptable salt thereof per day. In some embodiments, the method comprises administering to the patient about 165 mg of the compound of Formula (I) or pharmaceutically acceptable salt thereof per day. In some embodiments, the method comprises administering to the patient about 170 mg of the compound of Formula (I) or pharmaceutically acceptable salt thereof per day. In some embodiments, the method comprises administering to the patient about 175 mg of the compound of Formula (I) or pharmaceutically acceptable salt thereof per day. In some embodiments, the method comprises administering to the patient about 180 mg of the compound of Formula (I) or pharmaceutically acceptable salt thereof per day. In some embodiments, the method comprises administering to the patient about 185 mg of the compound of Formula (I) or pharmaceutically acceptable salt thereof per day. In some embodiments, the method comprises administering to the patient about 190 mg of the compound of Formula (I) or pharmaceutically acceptable salt thereof per day. In some embodiments, the method comprises administering to the patient about 195 mg of the compound of Formula (I) or pharmaceutically acceptable salt thereof per day. In some embodiments, the method comprises administering to the patient about 200 mg of the compound of Formula (I) or pharmaceutically acceptable salt thereof per day.

In some embodiments, the method comprises administering to the patient about 50 mg to about 200 mg of the compound of Formula (I) or pharmaceutically acceptable salt thereof once per day. In some embodiments, the method comprises administering to the patient about 50 mg to about 150 mg of the compound of Formula (I) or pharmaceutically acceptable salt thereof once per day. In some embodiments, the method comprises administering to the patient about 50 mg to about 100 mg of the compound of Formula (I) or pharmaceutically acceptable salt thereof once per day. In some embodiments, the method comprises administering to the patient about 60 mg to about 200 mg of the compound of Formula (I) or pharmaceutically acceptable salt thereof once per day. In some embodiments, the method comprises administering to the patient about 60 mg to about 150 mg of the compound of Formula (I) or pharmaceutically acceptable salt thereof once per day. In some embodiments, the method comprises administering to the patient about 60 mg to about 100 mg of the compound of Formula (I) or pharmaceutically acceptable salt thereof once per day. In some embodiments, the method comprises administering to the patient about 70 mg to about 200 mg of the compound of Formula (I) or pharmaceutically acceptable salt thereof once per day. In some embodiments, the method comprises administering to the patient about 70 mg to about 150 mg of the compound of Formula (I) or pharmaceutically acceptable salt thereof once per day. In some embodiments, the method comprises administering to the patient about 70 mg to about 100 mg of the compound of Formula (I) or pharmaceutically acceptable salt thereof once per day. In some embodiments, the method comprises administering to the patient about 80 mg to about 200 mg of the compound of Formula (I) or pharmaceutically acceptable salt thereof once per day. In some embodiments, the method comprises administering to the patient about 80 mg to about 150 mg of the compound of Formula (I) or pharmaceutically acceptable salt thereof once per day. In some embodiments, the method comprises administering to the patient about 80 mg to about 100 mg of the compound of Formula (I) or pharmaceutically acceptable salt thereof once per day. In some embodiments, the method comprises administering to the patient about 90 mg to about 200 mg of the compound of Formula (I) or pharmaceutically acceptable salt thereof once per day. In some embodiments, the method comprises administering to the patient about 90 mg to about 150 mg of the compound of Formula (I) or pharmaceutically acceptable salt thereof once per day. In some embodiments, the method comprises administering to the patient about 90 mg to about 100 mg of the compound of Formula (I) or pharmaceutically acceptable salt thereof once per day. In some embodiments, the method comprises administering to the patient about 100 mg to about 200 mg of the compound of Formula (I) or pharmaceutically acceptable salt thereof once per day. In some embodiments, the method comprises administering to the patient about 100 mg to about 150 mg of the compound of Formula (I) or pharmaceutically acceptable salt thereof once per day.

In some embodiments, the method comprises administering to the patient about 50 mg of the compound of Formula (I) or pharmaceutically acceptable salt thereof once per day. In some embodiments, the method comprises administering to the patient about 55 mg of the compound of Formula (I) or pharmaceutically acceptable salt thereof once per day. In some embodiments, the method comprises administering to the patient about 60 mg of the compound of Formula (I) or pharmaceutically acceptable salt thereof once per day. In some embodiments, the method comprises administering to the patient about 65 mg of the compound of Formula (I) or pharmaceutically acceptable salt thereof once per day. In some embodiments, the method comprises administering to the patient about 70 mg of the compound of Formula (I) or pharmaceutically acceptable salt thereof once per day. In some embodiments, the method comprises administering to the patient about 75 mg of the compound of Formula (I) or pharmaceutically acceptable salt thereof once per day. In some embodiments, the method comprises administering to the patient about 80 mg of the compound of Formula (I) or pharmaceutically acceptable salt thereof once per day. In some embodiments, the method comprises administering to the patient about 85 mg of the compound of Formula (I) or pharmaceutically acceptable salt thereof once per day. In some embodiments, the method comprises administering to the patient about 90 mg of the compound of Formula (I) or pharmaceutically acceptable salt thereof once per day. In some embodiments, the method comprises administering to the patient about 95 mg of the compound of Formula (I) or pharmaceutically acceptable salt thereof once per day. In some embodiments, the method comprises administering to the patient about 100 mg of the compound of Formula (I) or pharmaceutically acceptable salt thereof once per day. In some embodiments, the method comprises administering to the patient about 105 mg of the compound of Formula (I) or pharmaceutically acceptable salt thereof once per day. In some embodiments, the method comprises administering to the patient about 110 mg of the compound of Formula (I) or pharmaceutically acceptable salt thereof once per day. In some embodiments, the method comprises administering to the patient about 115 mg of the compound of Formula (I) or pharmaceutically acceptable salt thereof once per day. In some embodiments, the method comprises administering to the patient about 120 mg of the compound of Formula (I) or pharmaceutically acceptable salt thereof once per day. In some embodiments, the method comprises administering to the patient about 125 mg of the compound of Formula (I) or pharmaceutically acceptable salt thereof once per day. In some embodiments, the method comprises administering to the patient about 130 mg of the compound of Formula (I) or pharmaceutically acceptable salt thereof once per day. In some embodiments, the method comprises administering to the patient about 135 mg of the compound of Formula (I) or pharmaceutically acceptable salt thereof once per day. In some embodiments, the method comprises administering to the patient about 140 mg of the compound of Formula (I) or pharmaceutically acceptable salt thereof once per day. In some embodiments, the method comprises administering to the patient about 145 mg of the compound of Formula (I) or pharmaceutically acceptable salt thereof once per day. In some embodiments, the method comprises administering to the patient about 150 mg of the compound of Formula (I) or pharmaceutically acceptable salt thereof once per day. In some embodiments, the method comprises administering to the patient about 155 mg of the compound of Formula (I) or pharmaceutically acceptable salt thereof once per day. In some embodiments, the method comprises administering to the patient about 160 mg of the compound of Formula (I) or pharmaceutically acceptable salt thereof once per day. In some embodiments, the method comprises administering to the patient about 165 mg of the compound of Formula (I) or pharmaceutically acceptable salt thereof once per day. In some embodiments, the method comprises administering to the patient about 170 mg of the compound of Formula (I) or pharmaceutically acceptable salt thereof once per day. In some embodiments, the method comprises administering to the patient about 175 mg of the compound of Formula (I) or pharmaceutically acceptable salt thereof once per day. In some embodiments, the method comprises administering to the patient about 180 mg of the compound of Formula (I) or pharmaceutically acceptable salt thereof once per day. In some embodiments, the method comprises administering to the patient about 185 mg of the compound of Formula (I) or pharmaceutically acceptable salt thereof once per day. In some embodiments, the method comprises administering to the patient about 190 mg of the compound of Formula (I) or pharmaceutically acceptable salt thereof once per day. In some embodiments, the method comprises administering to the patient about 195 mg of the compound of Formula (I) or pharmaceutically acceptable salt thereof once per day. In some embodiments, the method comprises administering to the patient about 200 mg of the compound of Formula (I) or pharmaceutically acceptable salt thereof once per day.

In some embodiments, the method comprises administering to the patient about 50 mg to about 100 mg of the compound of Formula (I) or pharmaceutically acceptable salt thereof twice per day. In some embodiments, the method comprises administering to the patient about 60 mg to about 100 mg of the compound of Formula (I) or pharmaceutically acceptable salt thereof twice per day. In some embodiments, the method comprises administering to the patient about 70 mg to about 100 mg of the compound of Formula (I) or pharmaceutically acceptable salt thereof twice per day. In some embodiments, the method comprises administering to the patient about 80 mg to about 100 mg of the compound of Formula (I) or pharmaceutically acceptable salt thereof twice per day. In some embodiments, the method comprises administering to the patient about 90 mg to about 100 mg of the compound of Formula (I) or pharmaceutically acceptable salt thereof twice per day.

In some embodiments, the method comprises administering to the patient about 50 mg of the compound of Formula (I) or pharmaceutically acceptable salt thereof twice per day. In some embodiments, the method comprises administering to the patient about 55 mg of the compound of Formula (I) or pharmaceutically acceptable salt thereof twice per day. In some embodiments, the method comprises administering to the patient about 60 mg of the compound of Formula (I) or pharmaceutically acceptable salt thereof twice per day. In some embodiments, the method comprises administering to the patient about 65 mg of the compound of Formula (I) or pharmaceutically acceptable salt thereof twice per day. In some embodiments, the method comprises administering to the patient about 70 mg of the compound of Formula (I) or pharmaceutically acceptable salt thereof twice per day. In some embodiments, the method comprises administering to the patient about 75 mg of the compound of Formula (I) or pharmaceutically acceptable salt thereof twice per day. In some embodiments, the method comprises administering to the patient about 80 mg of the compound of Formula (I) or pharmaceutically acceptable salt thereof twice per day. In some embodiments, the method comprises administering to the patient about 85 mg of the compound of Formula (I) or pharmaceutically acceptable salt thereof twice per day. In some embodiments, the method comprises administering to the patient about 90 mg of the compound of Formula (I) or pharmaceutically acceptable salt thereof twice per day. In some embodiments, the method comprises administering to the patient about 95 mg of the compound of Formula (I) or pharmaceutically acceptable salt thereof twice per day. In some embodiments, the method comprises administering to the patient about 100 mg of the compound of Formula (I) or pharmaceutically acceptable salt thereof twice per day.

The compound of Formula (I) or pharmaceutically acceptable salt thereof may, in another embodiment, be administered to the patient on an intermittent basis in the methods for achieving at least a partial response in a patient having pancreatic ductal adenocarcinoma. In some embodiments, the compound of Formula (I) or pharmaceutically acceptable salt thereof is administered the patient every other day. In some embodiments, the compound of Formula (I) or pharmaceutically acceptable salt thereof is administered the patient every other day. In some embodiments, the compound of Formula (I) or pharmaceutically acceptable salt thereof is administered the patient once per week. In some embodiments, the compound of Formula (I) or pharmaceutically acceptable salt thereof is administered the patient twice per week. In some embodiments, the compound of Formula (I) or pharmaceutically acceptable salt thereof is administered the patient three times per week.

In some embodiments, the method comprises administering to the patient about 50 mg to about 200 mg of the compound of Formula (I) or pharmaceutically acceptable salt thereof once a week. In some embodiments, the method comprises administering to the patient about 50 mg to about 150 mg of the compound of Formula (I) or pharmaceutically acceptable salt thereof once a week. In some embodiments, the method comprises administering to the patient about 50 mg to about 100 mg of the compound of Formula (I) or pharmaceutically acceptable salt thereof once a week. In some embodiments, the method comprises administering to the patient about 60 mg to about 200 mg of the compound of Formula (I) or pharmaceutically acceptable salt thereof once a week. In some embodiments, the method comprises administering to the patient about 60 mg to about 150 mg of the compound of Formula (I) or pharmaceutically acceptable salt thereof once a week. In some embodiments, the method comprises administering to the patient about 60 mg to about 100 mg of the compound of Formula (I) or pharmaceutically acceptable salt thereof once a week. In some embodiments, the method comprises administering to the patient about 70 mg to about 200 mg of the compound of Formula (I) or pharmaceutically acceptable salt thereof once a week. In some embodiments, the method comprises administering to the patient about 70 mg to about 150 mg of the compound of Formula (I) or pharmaceutically acceptable salt thereof once a week. In some embodiments, the method comprises administering to the patient about 70 mg to about 100 mg of the compound of Formula (I) or pharmaceutically acceptable salt thereof once a week. In some embodiments, the method comprises administering to the patient about 80 mg to about 200 mg of the compound of Formula (I) or pharmaceutically acceptable salt thereof once a week. In some embodiments, the method comprises administering to the patient about 80 mg to about 150 mg of the compound of Formula (I) or pharmaceutically acceptable salt thereof once a week. In some embodiments, the method comprises administering to the patient about 80 mg to about 100 mg of the compound of Formula (I) or pharmaceutically acceptable salt thereof once a week. In some embodiments, the method comprises administering to the patient about 90 mg to about 200 mg of the compound of Formula (I) or pharmaceutically acceptable salt thereof once a week. In some embodiments, the method comprises administering to the patient about 90 mg to about 150 mg of the compound of Formula (I) or pharmaceutically acceptable salt thereof once a week. In some embodiments, the method comprises administering to the patient about 90 mg to about 100 mg of the compound of Formula (I) or pharmaceutically acceptable salt thereof once a week. In some embodiments, the method comprises administering to the patient about 100 mg to about 200 mg of the compound of Formula (I) or pharmaceutically acceptable salt thereof once a week. In some embodiments, the method comprises administering to the patient about 100 mg to about 150 mg of the compound of Formula (I) or pharmaceutically acceptable salt thereof once a week.

In some embodiments, the method comprises administering to the patient about 50 mg of the compound of Formula (I) or pharmaceutically acceptable salt thereof once a week. In some embodiments, the method comprises administering to the patient about 55 mg of the compound of Formula (I) or pharmaceutically acceptable salt thereof once a week. In some embodiments, the method comprises administering to the patient about 60 mg of the compound of Formula (I) or pharmaceutically acceptable salt thereof once a week. In some embodiments, the method comprises administering to the patient about 65 mg of the compound of Formula (I) or pharmaceutically acceptable salt thereof once a week. In some embodiments, the method comprises administering to the patient about 70 mg of the compound of Formula (I) or pharmaceutically acceptable salt thereof once a week. In some embodiments, the method comprises administering to the patient about 75 mg of the compound of Formula (I) or pharmaceutically acceptable salt thereof once a week. In some embodiments, the method comprises administering to the patient about 80 mg of the compound of Formula (I) or pharmaceutically acceptable salt thereof once a week. In some embodiments, the method comprises administering to the patient about 85 mg of the compound of Formula (I) or pharmaceutically acceptable salt thereof once a week. In some embodiments, the method comprises administering to the patient about 90 mg of the compound of Formula (I) or pharmaceutically acceptable salt thereof once a week. In some embodiments, the method comprises administering to the patient about 95 mg of the compound of Formula (I) or pharmaceutically acceptable salt thereof once a week. In some embodiments, the method comprises administering to the patient about 100 mg of the compound of Formula (I) or pharmaceutically acceptable salt thereof once a week. In some embodiments, the method comprises administering to the patient about 105 mg of the compound of Formula (I) or pharmaceutically acceptable salt thereof once a week. In some embodiments, the method comprises administering to the patient about 110 mg of the compound of Formula (I) or pharmaceutically acceptable salt thereof once a week. In some embodiments, the method comprises administering to the patient about 115 mg of the compound of Formula (I) or pharmaceutically acceptable salt thereof once a week. In some embodiments, the method comprises administering to the patient about 120 mg of the compound of Formula (I) or pharmaceutically acceptable salt thereof once a week. In some embodiments, the method comprises administering to the patient about 125 mg of the compound of Formula (I) or pharmaceutically acceptable salt thereof once a week. In some embodiments, the method comprises administering to the patient about 130 mg of the compound of Formula (I) or pharmaceutically acceptable salt thereof once a week. In some embodiments, the method comprises administering to the patient about 135 mg of the compound of Formula (I) or pharmaceutically acceptable salt thereof once a week. In some embodiments, the method comprises administering to the patient about 140 mg of the compound of Formula (I) or pharmaceutically acceptable salt thereof once a week. In some embodiments, the method comprises administering to the patient about 145 mg of the compound of Formula (I) or pharmaceutically acceptable salt thereof once a week. In some embodiments, the method comprises administering to the patient about 150 mg of the compound of Formula (I) or pharmaceutically acceptable salt thereof once a week. In some embodiments, the method comprises administering to the patient about 155 mg of the compound of Formula (I) or pharmaceutically acceptable salt thereof once a week. In some embodiments, the method comprises administering to the patient about 160 mg of the compound of Formula (I) or pharmaceutically acceptable salt thereof once a week. In some embodiments, the method comprises administering to the patient about 165 mg of the compound of Formula (I) or pharmaceutically acceptable salt thereof once a week. In some embodiments, the method comprises administering to the patient about 170 mg of the compound of Formula (I) or pharmaceutically acceptable salt thereof once a week. In some embodiments, the method comprises administering to the patient about 175 mg of the compound of Formula (I) or pharmaceutically acceptable salt thereof once a week. In some embodiments, the method comprises administering to the patient about 180 mg of the compound of Formula (I) or pharmaceutically acceptable salt thereof once a week. In some embodiments, the method comprises administering to the patient about 185 mg of the compound of Formula (I) or pharmaceutically acceptable salt thereof once a week. In some embodiments, the method comprises administering to the patient about 190 mg of the compound of Formula (I) or pharmaceutically acceptable salt thereof once a week. In some embodiments, the method comprises administering to the patient about 195 mg of the compound of Formula (I) or pharmaceutically acceptable salt thereof once a week. In some embodiments, the method comprises administering to the patient about 200 mg of the compound of Formula (I) or pharmaceutically acceptable salt thereof once a week.

In some embodiments, the method comprises administering to the patient about 50 mg to about 200 mg of the compound of Formula (I) or pharmaceutically acceptable salt thereof twice a week. In some embodiments, the method comprises administering to the patient about 50 mg to about 150 mg of the compound of Formula (I) or pharmaceutically acceptable salt thereof twice a week. In some embodiments, the method comprises administering to the patient about 50 mg to about 100 mg of the compound of Formula (I) or pharmaceutically acceptable salt thereof twice a week. In some embodiments, the method comprises administering to the patient about 60 mg to about 200 mg of the compound of Formula (I) or pharmaceutically acceptable salt thereof twice a week. In some embodiments, the method comprises administering to the patient about 60 mg to about 150 mg of the compound of Formula (I) or pharmaceutically acceptable salt thereof twice a week. In some embodiments, the method comprises administering to the patient about 60 mg to about 100 mg of the compound of Formula (I) or pharmaceutically acceptable salt thereof twice a week. In some embodiments, the method comprises administering to the patient about 70 mg to about 200 mg of the compound of Formula (I) or pharmaceutically acceptable salt thereof twice a week. In some embodiments, the method comprises administering to the patient about 70 mg to about 150 mg of the compound of Formula (I) or pharmaceutically acceptable salt thereof twice a week. In some embodiments, the method comprises administering to the patient about 70 mg to about 100 mg of the compound of Formula (I) or pharmaceutically acceptable salt thereof twice a week. In some embodiments, the method comprises administering to the patient about 80 mg to about 200 mg of the compound of Formula (I) or pharmaceutically acceptable salt thereof twice a week. In some embodiments, the method comprises administering to the patient about 80 mg to about 150 mg of the compound of Formula (I) or pharmaceutically acceptable salt thereof twice a week. In some embodiments, the method comprises administering to the patient about 80 mg to about 100 mg of the compound of Formula (I) or pharmaceutically acceptable salt thereof twice a week. In some embodiments, the method comprises administering to the patient about 90 mg to about 200 mg of the compound of Formula (I) or pharmaceutically acceptable salt thereof twice a week. In some embodiments, the method comprises administering to the patient about 90 mg to about 150 mg of the compound of Formula (I) or pharmaceutically acceptable salt thereof twice a week. In some embodiments, the method comprises administering to the patient about 90 mg to about 100 mg of the compound of Formula (I) or pharmaceutically acceptable salt thereof twice a week. In some embodiments, the method comprises administering to the patient about 100 mg to about 200 mg of the compound of Formula (I) or pharmaceutically acceptable salt thereof twice a week. In some embodiments, the method comprises administering to the patient about 100 mg to about 150 mg of the compound of Formula (I) or pharmaceutically acceptable salt thereof twice a week.

In some embodiments, the method comprises administering to the patient about 50 mg of the compound of Formula (I) or pharmaceutically acceptable salt thereof twice a week. In some embodiments, the method comprises administering to the patient about 55 mg of the compound of Formula (I) or pharmaceutically acceptable salt thereof twice a week. In some embodiments, the method comprises administering to the patient about 60 mg of the compound of Formula (I) or pharmaceutically acceptable salt thereof twice a week. In some embodiments, the method comprises administering to the patient about 65 mg of the compound of Formula (I) or pharmaceutically acceptable salt thereof twice a week. In some embodiments, the method comprises administering to the patient about 70 mg of the compound of Formula (I) or pharmaceutically acceptable salt thereof twice a week. In some embodiments, the method comprises administering to the patient about 75 mg of the compound of Formula (I) or pharmaceutically acceptable salt thereof twice a week. In some embodiments, the method comprises administering to the patient about 80 mg of the compound of Formula (I) or pharmaceutically acceptable salt thereof twice a week. In some embodiments, the method comprises administering to the patient about 85 mg of the compound of Formula (I) or pharmaceutically acceptable salt thereof twice a week. In some embodiments, the method comprises administering to the patient about 90 mg of the compound of Formula (I) or pharmaceutically acceptable salt thereof twice a week. In some embodiments, the method comprises administering to the patient about 95 mg of the compound of Formula (I) or pharmaceutically acceptable salt thereof twice a week. In some embodiments, the method comprises administering to the patient about 100 mg of the compound of Formula (I) or pharmaceutically acceptable salt thereof twice a week. In some embodiments, the method comprises administering to the patient about 105 mg of the compound of Formula (I) or pharmaceutically acceptable salt thereof twice a week. In some embodiments, the method comprises administering to the patient about 110 mg of the compound of Formula (I) or pharmaceutically acceptable salt thereof twice a week. In some embodiments, the method comprises administering to the patient about 115 mg of the compound of Formula (I) or pharmaceutically acceptable salt thereof twice a week. In some embodiments, the method comprises administering to the patient about 120 mg of the compound of Formula (I) or pharmaceutically acceptable salt thereof twice a week. In some embodiments, the method comprises administering to the patient about 125 mg of the compound of Formula (I) or pharmaceutically acceptable salt thereof twice a week. In some embodiments, the method comprises administering to the patient about 130 mg of the compound of Formula (I) or pharmaceutically acceptable salt thereof twice a week. In some embodiments, the method comprises administering to the patient about 135 mg of the compound of Formula (I) or pharmaceutically acceptable salt thereof twice a week. In some embodiments, the method comprises administering to the patient about 140 mg of the compound of Formula (I) or pharmaceutically acceptable salt thereof twice a week. In some embodiments, the method comprises administering to the patient about 145 mg of the compound of Formula (I) or pharmaceutically acceptable salt thereof twice a week. In some embodiments, the method comprises administering to the patient about 150 mg of the compound of Formula (I) or pharmaceutically acceptable salt thereof twice a week. In some embodiments, the method comprises administering to the patient about 155 mg of the compound of Formula (I) or pharmaceutically acceptable salt thereof twice a week. In some embodiments, the method comprises administering to the patient about 160 mg of the compound of Formula (I) or pharmaceutically acceptable salt thereof twice a week. In some embodiments, the method comprises administering to the patient about 165 mg of the compound of Formula (I) or pharmaceutically acceptable salt thereof twice a week. In some embodiments, the method comprises administering to the patient about 170 mg of the compound of Formula (I) or pharmaceutically acceptable salt thereof twice a week. In some embodiments, the method comprises administering to the patient about 175 mg of the compound of Formula (I) or pharmaceutically acceptable salt thereof twice a week. In some embodiments, the method comprises administering to the patient about 180 mg of the compound of Formula (I) or pharmaceutically acceptable salt thereof twice a week. In some embodiments, the method comprises administering to the patient about 185 mg of the compound of Formula (I) or pharmaceutically acceptable salt thereof twice a week. In some embodiments, the method comprises administering to the patient about 190 mg of the compound of Formula (I) or pharmaceutically acceptable salt thereof twice a week. In some embodiments, the method comprises administering to the patient about 195 mg of the compound of Formula (I) or pharmaceutically acceptable salt thereof twice a week. In some embodiments, the method comprises administering to the patient about 200 mg of the compound of Formula (I) or pharmaceutically acceptable salt thereof twice a week.

In some embodiments, the method comprises administering to the patient about 50 mg to about 200 mg of the compound of Formula (I) or pharmaceutically acceptable salt thereof three times a week. In some embodiments, the method comprises administering to the patient about 50 mg to about 150 mg of the compound of Formula (I) or pharmaceutically acceptable salt thereof three times a week. In some embodiments, the method comprises administering to the patient about 50 mg to about 100 mg of the compound of Formula (I) or pharmaceutically acceptable salt thereof three times a week. In some embodiments, the method comprises administering to the patient about 60 mg to about 200 mg of the compound of Formula (I) or pharmaceutically acceptable salt thereof three times a week. In some embodiments, the method comprises administering to the patient about 60 mg to about 150 mg of the compound of Formula (I) or pharmaceutically acceptable salt thereof three times a week. In some embodiments, the method comprises administering to the patient about 60 mg to about 100 mg of the compound of Formula (I) or pharmaceutically acceptable salt thereof three times a week. In some embodiments, the method comprises administering to the patient about 70 mg to about 200 mg of the compound of Formula (I) or pharmaceutically acceptable salt thereof three times a week. In some embodiments, the method comprises administering to the patient about 70 mg to about 150 mg of the compound of Formula (I) or pharmaceutically acceptable salt thereof three times a week. In some embodiments, the method comprises administering to the patient about 70 mg to about 100 mg of the compound of Formula (I) or pharmaceutically acceptable salt thereof three times a week. In some embodiments, the method comprises administering to the patient about 80 mg to about 200 mg of the compound of Formula (I) or pharmaceutically acceptable salt thereof three times a week. In some embodiments, the method comprises administering to the patient about 80 mg to about 150 mg of the compound of Formula (I) or pharmaceutically acceptable salt thereof three times a week. In some embodiments, the method comprises administering to the patient about 80 mg to about 100 mg of the compound of Formula (I) or pharmaceutically acceptable salt thereof three times a week. In some embodiments, the method comprises administering to the patient about 90 mg to about 200 mg of the compound of Formula (I) or pharmaceutically acceptable salt thereof three times a week. In some embodiments, the method comprises administering to the patient about 90 mg to about 150 mg of the compound of Formula (I) or pharmaceutically acceptable salt thereof three times a week. In some embodiments, the method comprises administering to the patient about 90 mg to about 100 mg of the compound of Formula (I) or pharmaceutically acceptable salt thereof three times a week. In some embodiments, the method comprises administering to the patient about 100 mg to about 200 mg of the compound of Formula (I) or pharmaceutically acceptable salt thereof three times a week. In some embodiments, the method comprises administering to the patient about 100 mg to about 150 mg of the compound of Formula (I) or pharmaceutically acceptable salt thereof three times a week.

In some embodiments, the method comprises administering to the patient about 50 mg of the compound of Formula (I) or pharmaceutically acceptable salt thereof three times a week. In some embodiments, the method comprises administering to the patient about 55 mg of the compound of Formula (I) or pharmaceutically acceptable salt thereof three times a week. In some embodiments, the method comprises administering to the patient about 60 mg of the compound of Formula (I) or pharmaceutically acceptable salt thereof three times a week. In some embodiments, the method comprises administering to the patient about 65 mg of the compound of Formula (I) or pharmaceutically acceptable salt thereof three times a week. In some embodiments, the method comprises administering to the patient about 70 mg of the compound of Formula (I) or pharmaceutically acceptable salt thereof three times a week. In some embodiments, the method comprises administering to the patient about 75 mg of the compound of Formula (I) or pharmaceutically acceptable salt thereof three times a week. In some embodiments, the method comprises administering to the patient about 80 mg of the compound of Formula (I) or pharmaceutically acceptable salt thereof three times a week. In some embodiments, the method comprises administering to the patient about 85 mg of the compound of Formula (I) or pharmaceutically acceptable salt thereof three times a week. In some embodiments, the method comprises administering to the patient about 90 mg of the compound of Formula (I) or pharmaceutically acceptable salt thereof three times a week. In some embodiments, the method comprises administering to the patient about 95 mg of the compound of Formula (I) or pharmaceutically acceptable salt thereof three times a week. In some embodiments, the method comprises administering to the patient about 100 mg of the compound of Formula (I) or pharmaceutically acceptable salt thereof three times a week. In some embodiments, the method comprises administering to the patient about 105 mg of the compound of Formula (I) or pharmaceutically acceptable salt thereof three times a week. In some embodiments, the method comprises administering to the patient about 110 mg of the compound of Formula (I) or pharmaceutically acceptable salt thereof three times a week. In some embodiments, the method comprises administering to the patient about 115 mg of the compound of Formula (I) or pharmaceutically acceptable salt thereof three times a week. In some embodiments, the method comprises administering to the patient about 120 mg of the compound of Formula (I) or pharmaceutically acceptable salt thereof three times a week. In some embodiments, the method comprises administering to the patient about 125 mg of the compound of Formula (I) or pharmaceutically acceptable salt thereof three times a week. In some embodiments, the method comprises administering to the patient about 130 mg of the compound of Formula (I) or pharmaceutically acceptable salt thereof three times a week. In some embodiments, the method comprises administering to the patient about 135 mg of the compound of Formula (I) or pharmaceutically acceptable salt thereof three times a week. In some embodiments, the method comprises administering to the patient about 140 mg of the compound of Formula (I) or pharmaceutically acceptable salt thereof three times a week. In some embodiments, the method comprises administering to the patient about 145 mg of the compound of Formula (I) or pharmaceutically acceptable salt thereof three times a week. In some embodiments, the method comprises administering to the patient about 150 mg of the compound of Formula (I) or pharmaceutically acceptable salt thereof three times a week. In some embodiments, the method comprises administering to the patient about 155 mg of the compound of Formula (I) or pharmaceutically acceptable salt thereof three times a week. In some embodiments, the method comprises administering to the patient about 160 mg of the compound of Formula (I) or pharmaceutically acceptable salt thereof three times a week. In some embodiments, the method comprises administering to the patient about 165 mg of the compound of Formula (I) or pharmaceutically acceptable salt thereof three times a week. In some embodiments, the method comprises administering to the patient about 170 mg of the compound of Formula (I) or pharmaceutically acceptable salt thereof three times a week. In some embodiments, the method comprises administering to the patient about 175 mg of the compound of Formula (I) or pharmaceutically acceptable salt thereof three times a week. In some embodiments, the method comprises administering to the patient about 180 mg of the compound of Formula (I) or pharmaceutically acceptable salt thereof three times a week. In some embodiments, the method comprises administering to the patient about 185 mg of the compound of Formula (I) or pharmaceutically acceptable salt thereof three times a week. In some embodiments, the method comprises administering to the patient about 190 mg of the compound of Formula (I) or pharmaceutically acceptable salt thereof three times a week. In some embodiments, the method comprises administering to the patient about 195 mg of the compound of Formula (I) or pharmaceutically acceptable salt thereof three times a week. In some embodiments, the method comprises administering to the patient about 200 mg of the compound of Formula (I) or pharmaceutically acceptable salt thereof three times a week.

In some embodiments, the method comprises administering to the patient about 50 mg to about 200 mg of the compound of Formula (I) or pharmaceutically acceptable salt thereof every other day. In some embodiments, the method comprises administering to the patient about 50 mg to about 150 mg of the compound of Formula (I) or pharmaceutically acceptable salt thereof every other day. In some embodiments, the method comprises administering to the patient about 50 mg to about 100 mg of the compound of Formula (I) or pharmaceutically acceptable salt thereof every other day. In some embodiments, the method comprises administering to the patient about 60 mg to about 200 mg of the compound of Formula (I) or pharmaceutically acceptable salt thereof every other day. In some embodiments, the method comprises administering to the patient about 60 mg to about 150 mg of the compound of Formula (I) or pharmaceutically acceptable salt thereof every other day. In some embodiments, the method comprises administering to the patient about 60 mg to about 100 mg of the compound of Formula (I) or pharmaceutically acceptable salt thereof every other day. In some embodiments, the method comprises administering to the patient about 70 mg to about 200 mg of the compound of Formula (I) or pharmaceutically acceptable salt thereof every other day. In some embodiments, the method comprises administering to the patient about 70 mg to about 150 mg of the compound of Formula (I) or pharmaceutically acceptable salt thereof every other day. In some embodiments, the method comprises administering to the patient about 70 mg to about 100 mg of the compound of Formula (I) or pharmaceutically acceptable salt thereof every other day. In some embodiments, the method comprises administering to the patient about 80 mg to about 200 mg of the compound of Formula (I) or pharmaceutically acceptable salt thereof every other day. In some embodiments, the method comprises administering to the patient about 80 mg to about 150 mg of the compound of Formula (I) or pharmaceutically acceptable salt thereof every other day. In some embodiments, the method comprises administering to the patient about 80 mg to about 100 mg of the compound of Formula (I) or pharmaceutically acceptable salt thereof every other day. In some embodiments, the method comprises administering to the patient about 90 mg to about 200 mg of the compound of Formula (I) or pharmaceutically acceptable salt thereof every other day. In some embodiments, the method comprises administering to the patient about 90 mg to about 150 mg of the compound of Formula (I) or pharmaceutically acceptable salt thereof every other day. In some embodiments, the method comprises administering to the patient about 90 mg to about 100 mg of the compound of Formula (I) or pharmaceutically acceptable salt thereof every other day. In some embodiments, the method comprises administering to the patient about 100 mg to about 200 mg of the compound of Formula (I) or pharmaceutically acceptable salt thereof every other day. In some embodiments, the method comprises administering to the patient about 100 mg to about 150 mg of the compound of Formula (I) or pharmaceutically acceptable salt thereof every other day.

In some embodiments, the method comprises administering to the patient about 50 mg of the compound of Formula (I) or pharmaceutically acceptable salt thereof every other day. In some embodiments, the method comprises administering to the patient about 55 mg of the compound of Formula (I) or pharmaceutically acceptable salt thereof every other day. In some embodiments, the method comprises administering to the patient about 60 mg of the compound of Formula (I) or pharmaceutically acceptable salt thereof every other day. In some embodiments, the method comprises administering to the patient about 65 mg of the compound of Formula (I) or pharmaceutically acceptable salt thereof every other day. In some embodiments, the method comprises administering to the patient about 70 mg of the compound of Formula (I) or pharmaceutically acceptable salt thereof every other day. In some embodiments, the method comprises administering to the patient about 75 mg of the compound of Formula (I) or pharmaceutically acceptable salt thereof every other day. In some embodiments, the method comprises administering to the patient about 80 mg of the compound of Formula (I) or pharmaceutically acceptable salt thereof every other day. In some embodiments, the method comprises administering to the patient about 85 mg of the compound of Formula (I) or pharmaceutically acceptable salt thereof every other day. In some embodiments, the method comprises administering to the patient about 90 mg of the compound of Formula (I) or pharmaceutically acceptable salt thereof every other day. In some embodiments, the method comprises administering to the patient about 95 mg of the compound of Formula (I) or pharmaceutically acceptable salt thereof every other day. In some embodiments, the method comprises administering to the patient about 100 mg of the compound of Formula (I) or pharmaceutically acceptable salt thereof every other day. In some embodiments, the method comprises administering to the patient about 105 mg of the compound of Formula (I) or pharmaceutically acceptable salt thereof every other day. In some embodiments, the method comprises administering to the patient about 110 mg of the compound of Formula (I) or pharmaceutically acceptable salt thereof every other day. In some embodiments, the method comprises administering to the patient about 115 mg of the compound of Formula (I) or pharmaceutically acceptable salt thereof every other day. In some embodiments, the method comprises administering to the patient about 120 mg of the compound of Formula (I) or pharmaceutically acceptable salt thereof every other day. In some embodiments, the method comprises administering to the patient about 125 mg of the compound of Formula (I) or pharmaceutically acceptable salt thereof every other day. In some embodiments, the method comprises administering to the patient about 130 mg of the compound embodiments, the method comprises administering to the patient about 135 mg of the compound of Formula (I) or pharmaceutically acceptable salt thereof every other day. In some embodiments, the method comprises administering to the patient about 140 mg of the compound of Formula (I) or pharmaceutically acceptable salt thereof every other day. In some embodiments, the method comprises administering to the patient about 145 mg of the compound of Formula (I) or pharmaceutically acceptable salt thereof every other day. In some embodiments, the method comprises administering to the patient about 150 mg of the compound of Formula (I) or pharmaceutically acceptable salt thereof every other day. In some embodiments, the method comprises administering to the patient about 155 mg of the compound of Formula (I) or pharmaceutically acceptable salt thereof every other day. In some embodiments, the method comprises administering to the patient about 160 mg of the compound of Formula (I) or pharmaceutically acceptable salt thereof every other day. In some embodiments, the method comprises administering to the patient about 165 mg of the compound of Formula (I) or pharmaceutically acceptable salt thereof every other day. In some embodiments, the method comprises administering to the patient about 170 mg of the compound of Formula (I) or pharmaceutically acceptable salt thereof every other day. In some embodiments, the method comprises administering to the patient about 175 mg of the compound of Formula (I) or pharmaceutically acceptable salt thereof every other day. In some embodiments, the method comprises administering to the patient about 180 mg of the compound of Formula (I) or pharmaceutically acceptable salt thereof every other day. In some embodiments, the method comprises administering to the patient about 185 mg of the compound of Formula (I) or pharmaceutically acceptable salt thereof every other day. In some embodiments, the method comprises administering to the patient about 190 mg of the compound of Formula (I) or pharmaceutically acceptable salt thereof every other day. In some embodiments, the method comprises administering to the patient about 195 mg of the compound of Formula (I) or pharmaceutically acceptable salt thereof every other day. In some embodiments, the method comprises administering to the patient about 200 mg of the compound of Formula (I) or pharmaceutically acceptable salt thereof every other day.

In some embodiments, the methods further comprise administering a combination agent to the patient in need thereof. In some embodiments, the combination agent is carboplatin. In some embodiments, the combination agent is paclitaxel. In some embodiments, the method further comprises administering pemetrexed to the patient in need thereof. In some embodiments, the method further comprises administering one or more combination agents to the patient in need thereof. In some embodiments, the method further comprises administering carboplatin and paclitaxel.

In some embodiments, the combination agent is intravenously administered to the patient in need thereof. The one or more combination agents administered to a patient in the methods of achieving at least a partial response in patients having pancreatic ductal adenocarcinoma described herein can be dosed to achieve various concentrations of the combination agents in the patient's plasma, which can be quantified by the area under the curve (AUC) for each combination agent administered, e.g., intravenously administered. In some embodiments, the combination agent, (e.g., carboplatin) is intravenously administered to the patient in an amount sufficient to provide an AUC of about 5 to about 7 (e.g., an AUC of about 5 or about 6). In some embodiments, the method comprises reducing each of the amount of the compound of Formula (I) or pharmaceutically acceptable salt thereof and the combination agent administered to the patient after one or more months of administering to the patient in need thereof.

In some embodiments, the paclitaxel is intravenously administered to the patient, e.g., by intravenous infusion. The intravenously administered paclitaxel can be administered to a patient at various dose levels. In some embodiments, the paclitaxel is administered to the patient at about 50 mg/m² to about 150 mg/m². In some embodiments, the paclitaxel is administered to the patient at about 50 mg/m² to about 100 mg/m². In some embodiments, the paclitaxel is administered to the patient at about 60 mg/m² to about 80 mg/m². In some embodiments, the paclitaxel is administered to the patient at about 60 mg/m². In some embodiments, the paclitaxel is administered to the patient at about 80 mg/m². In some embodiments, the paclitaxel is administered to the patient at about 135 mg/m². In some embodiments, the paclitaxel is administered to the patient at about 175 mg/m².

Furthermore, the paclitaxel can be administered to the patient at various frequencies. In some embodiments, the paclitaxel is administered to the patient once per week (e.g., at about 60 mg/m² once per week or about 80 mg/m² once per week). In some embodiments, the paclitaxel is administered to the patient once every three weeks (e.g., at about 135 mg/m² once every three weeks or about 175 mg/m² once every three weeks). In some embodiments, the paclitaxel is administered to the patient by intravenous infusion once per week. In some embodiments, the paclitaxel is administered to the patient by intravenous infusion once per week for three consecutive weeks. In some embodiments, the method comprises administering paclitaxel to the patient by intravenous infusion for more than one cycle, wherein each cycle comprises administering paclitaxel to the patient by intravenous infusion once per week for three consecutive weeks (e.g., once per week for three consecutive weeks at about 60 mg/m² or about 80 mg/m²). In some embodiments, the method comprises not administering paclitaxel to the patient for at least one week between each cycle.

In some embodiments, the paclitaxel is administered to the patient by infusion once every three weeks. In some embodiments, the paclitaxel is administered to the patient for about 60 minutes once per week. In some embodiments, the paclitaxel is administered to the patient for about 3 hours once per week. In some embodiments, the paclitaxel is administered to the patient for about 60 minutes once every three weeks. In some embodiments, the paclitaxel is administered to the patient for about 3 hours once every three weeks. In some embodiments, the paclitaxel is administered to the patient by intravenous infusion for about 60 minutes once per week. In some embodiments, the paclitaxel is administered to the patient by infusion for about 3 hours once per week. In some embodiments, the paclitaxel is administered to the patient by intravenous infusion for about 60 minutes once every three weeks. In some embodiments, the paclitaxel is administered to the patient by infusion for about 3 hours once every three weeks. In some embodiments, the paclitaxel is continuously administered to the patient for about 60 minutes once per week. In some embodiments, the paclitaxel is continuously administered to the patient for about 3 hours once per week. In some embodiments, the paclitaxel is continuously administered to the patient for about 60 minutes once every three weeks. In some embodiments, the paclitaxel is continuously administered to the patient for about 3 hours once every three weeks. In some embodiments, the paclitaxel is continuously administered to the patient by infusion for about 60 minutes once per week. In some embodiments, the paclitaxel is continuously administered to the patient by intravenous infusion for about 3 hours once per week. In some embodiments, the paclitaxel is continuously administered to the patient by infusion for about 60 minutes once every three weeks. In some embodiments, the paclitaxel is continuously administered to the patient by intravenous infusion for about 3 hours once every three weeks.

The method of achieving at least a partial response in patients having pancreatic ductal adenocarcinoma described herein described herein can further comprise reducing each of the amount of the compound of Formula (I) or pharmaceutically acceptable salt thereof and the combination agent administered to the patient after one or more months of administering said compound of Formula (I) or pharmaceutically acceptable salt thereof and said combination agent to the patient in need thereof.

In embodiments of the methods of achieving at least a partial response in a patient having an pancreatic ductal adenocarcinoma described herein, the disclosure also contemplates administration of the compound of Formula (I) or a pharmaceutically acceptable salt to a patient in need thereof prior to (neo-adjuvant) or after (adjuvant) surgery (e.g., surgical treatment of a solid tumor). In some embodiments, the compound of Formula (I) or a pharmaceutically acceptable salt is administered as an adjuvant. In some embodiments, the compound of Formula (I) or a pharmaceutically acceptable salt is administered as a neo-adjuvant. In some embodiments, the compound of Formula (I) or a pharmaceutically acceptable salt is administered as a neo-adjuvant and an adjuvant. In some embodiments, the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 100 years. In some embodiments, administration of the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 5 years. In some embodiments, administration of the compound of Formula (I) or a pharmaceutically acceptable salt is administered to a patient in need thereof as a neo-adjuvant for a period of 3 months to 6 months, followed by administration of the compound of Formula (I) or a pharmaceutically acceptable salt as an adjuvant for a period from 1 day to 5 years. In some embodiments, no administration of the compound of Formula (I) or a pharmaceutically acceptable salt occurs prior to surgery. In some embodiments, the compound of Formula (I) or a pharmaceutically acceptable salt is administered as an adjuvant for a period from 1 day to 100 years. In some embodiments, the compound of Formula (I) or a pharmaceutically acceptable salt is administered as an adjuvant for a period from 1 day to 5 years.

In another embodiment, provided herein is a method for achieving a stable disease condition in a patient having pancreatic ductal adenocarcinoma, comprising administering to the patient an effective amount of the compound of Formula (I)

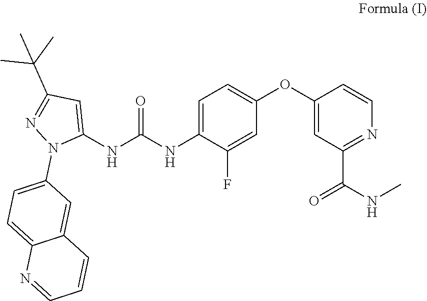 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In some embodiments, the method further comprises administering a combination agent to the patient in need thereof. In some embodiments, the combination agent is carboplatin. In some embodiments, the combination agent is paclitaxel. In some embodiments, the method further comprises administering pemetrexed to the patient in need thereof. In some embodiments, the method further comprises administering one or more combination agents to the patient in need thereof. In some embodiments, the method further comprises administering carboplatin and paclitaxel.

In some embodiments, the patient has previously been administered at least one prior line of therapy. In some embodiments, the therapy is a platinum-based therapy. In some embodiments, the patient previously been administered carboplatin and/or pemetrexed. In some embodiments, the patient previously been administered cisplatin and/or pemetrexed.

In embodiments of the methods for achieving a stable disease condition in a patient having pancreatic ductal adenocarcinoma described herein, the compound of Formula (I) or pharmaceutically acceptable salt thereof can be orally administered to the patient in need thereof. In certain embodiments, the compound of Formula (I) or pharmaceutically acceptable salt thereof is administered to the patient daily. In some embodiments, the method comprises administering to the patient about 50 mg to about 200 mg of the compound of Formula (I) or pharmaceutically acceptable salt thereof per day. In some embodiments, the method comprises administering to the patient about 50 mg to about 150 mg of the compound of Formula (I) or pharmaceutically acceptable salt thereof per day. In some embodiments, the method comprises administering to the patient about 50 mg to about 100 mg of the compound of Formula (I) or pharmaceutically acceptable salt thereof per day. In some embodiments, the method comprises administering to the patient about 60 mg to about 200 mg of the compound of Formula (I) or pharmaceutically acceptable salt thereof per day. In some embodiments, the method comprises administering to the patient about 60 mg to about 150 mg of the compound of Formula (I) or pharmaceutically acceptable salt thereof per day. In some embodiments, the method comprises administering to the patient about 60 mg to about 100 mg of the compound of Formula (I) or pharmaceutically acceptable salt thereof per day. In some embodiments, the method comprises administering to the patient about 70 mg to about 200 mg of the compound of Formula (I) or pharmaceutically acceptable salt thereof per day. In some embodiments, the method comprises administering to the patient about 70 mg to about 150 mg of the compound of Formula (I) or pharmaceutically acceptable salt thereof per day. In some embodiments, the method comprises administering to the patient about 70 mg to about 100 mg of the compound of Formula (I) or pharmaceutically acceptable salt thereof per day. In some embodiments, the method comprises administering to the patient about 80 mg to about 200 mg of the compound of Formula (I) or pharmaceutically acceptable salt thereof per day. In some embodiments, the method comprises administering to the patient about 80 mg to about 150 mg of the compound of Formula (I) or pharmaceutically acceptable salt thereof per day. In some embodiments, the method comprises administering to the patient about 80 mg to about 100 mg of the compound of Formula (I) or pharmaceutically acceptable salt thereof per day. In some embodiments, the method comprises administering to the patient about 90 mg to about 200 mg of the compound of Formula (I) or pharmaceutically acceptable salt thereof per day. In some embodiments, the method comprises administering to the patient about 90 mg to about 150 mg of the compound of Formula (I) or pharmaceutically acceptable salt thereof per day. In some embodiments, the method comprises administering to the patient about 90 mg to about 100 mg of the compound of Formula (I) or pharmaceutically acceptable salt thereof per day. In some embodiments, the method comprises administering to the patient about 100 mg to about 200 mg of the compound of Formula (I) or pharmaceutically acceptable salt thereof per day. In some embodiments, the method comprises administering to the patient about 100 mg to about 150 mg of the compound of Formula (I) or pharmaceutically acceptable salt thereof per day.

In some embodiments, the method comprises administering to the patient about 50 mg of the compound of Formula (I) or pharmaceutically acceptable salt thereof per day. In some embodiments, the method comprises administering to the patient about 55 mg of the compound of Formula (I) or pharmaceutically acceptable salt thereof per day. In some embodiments, the method comprises administering to the patient about 60 mg of the compound of Formula (I) or pharmaceutically acceptable salt thereof per day. In some embodiments, the method comprises administering to the patient about 65 mg of the compound of Formula (I) or pharmaceutically acceptable salt thereof per day. In some embodiments, the method comprises administering to the patient about 70 mg of the compound of Formula (I) or pharmaceutically acceptable salt thereof per day. In some embodiments, the method comprises administering to the patient about 75 mg of the compound of Formula (I) or pharmaceutically acceptable salt thereof per day. In some embodiments, the method comprises administering to the patient about 80 mg of the compound of Formula (I) or pharmaceutically acceptable salt thereof per day. In some embodiments, the method comprises administering to the patient about 85 mg of the compound of Formula (I) or pharmaceutically acceptable salt thereof per day. In some embodiments, the method comprises administering to the patient about 90 mg of the compound of Formula (I) or pharmaceutically acceptable salt thereof per day. In some embodiments, the method comprises administering to the patient about 95 mg of the compound of Formula (I) or pharmaceutically acceptable salt thereof per day. In some embodiments, the method comprises administering to the patient about 100 mg of the compound of Formula (I) or pharmaceutically acceptable salt thereof per day. In some embodiments, the method comprises administering to the patient about 105 mg of the compound of Formula (I) or pharmaceutically acceptable salt thereof per day. In some embodiments, the method comprises administering to the patient about 110 mg of the compound of Formula (I) or pharmaceutically acceptable salt thereof per day. In some embodiments, the method comprises administering to the patient about 115 mg of the compound of Formula (I) or pharmaceutically acceptable salt thereof per day. In some embodiments, the method comprises administering to the patient about 120 mg of the compound of Formula (I) or pharmaceutically acceptable salt thereof per day. In some embodiments, the method comprises administering to the patient about 125 mg of the compound of Formula (I) or pharmaceutically acceptable salt thereof per day. In some embodiments, the method comprises administering to the patient about 130 mg of the compound of Formula (I) or pharmaceutically acceptable salt thereof per day. In some embodiments, the method comprises administering to the patient about 135 mg of the compound of Formula (I) or pharmaceutically acceptable salt thereof per day. In some embodiments, the method comprises administering to the patient about 140 mg of the compound of Formula (I) or pharmaceutically acceptable salt thereof per day. In some embodiments, the method comprises administering to the patient about 145 mg of the compound of Formula (I) or pharmaceutically acceptable salt thereof per day. In some embodiments, the method comprises administering to the patient about 150 mg of the compound of Formula (I) or pharmaceutically acceptable salt thereof per day. In some embodiments, the method comprises administering to the patient about 155 mg of the compound of Formula (I) or pharmaceutically acceptable salt thereof per day. In some embodiments, the method comprises administering to the patient about 160 mg of the compound of Formula (I) or pharmaceutically acceptable salt thereof per day. In some embodiments, the method comprises administering to the patient about 165 mg of the compound of Formula (I) or pharmaceutically acceptable salt thereof per day. In some embodiments, the method comprises administering to the patient about 170 mg of the compound of Formula (I) or pharmaceutically acceptable salt thereof per day. In some embodiments, the method comprises administering to the patient about 175 mg of the compound of Formula (I) or pharmaceutically acceptable salt thereof per day. In some embodiments, the method comprises administering to the patient about 180 mg of the compound of Formula (I) or pharmaceutically acceptable salt thereof per day. In some embodiments, the method comprises administering to the patient about 185 mg of the compound of Formula (I) or pharmaceutically acceptable salt thereof per day. In some embodiments, the method comprises administering to the patient about 190 mg of the compound of Formula (I) or pharmaceutically acceptable salt thereof per day. In some embodiments, the method comprises administering to the patient about 195 mg of the compound of Formula (I) or pharmaceutically acceptable salt thereof per day. In some embodiments, the method comprises administering to the patient about 200 mg of the compound of Formula (I) or pharmaceutically acceptable salt thereof per day.

In some embodiments, the method comprises administering to the patient about 50 mg to about 200 mg of the compound of Formula (I) or pharmaceutically acceptable salt thereof once per day. In some embodiments, the method comprises administering to the patient about 50 mg to about 150 mg of the compound of Formula (I) or pharmaceutically acceptable salt thereof once per day. In some embodiments, the method comprises administering to the patient about 50 mg to about 100 mg of the compound of Formula (I) or pharmaceutically acceptable salt thereof once per day. In some embodiments, the method comprises administering to the patient about 60 mg to about 200 mg of the compound of Formula (I) or pharmaceutically acceptable salt thereof once per day. In some embodiments, the method comprises administering to the patient about 60 mg to about 150 mg of the compound of Formula (I) or pharmaceutically acceptable salt thereof once per day. In some embodiments, the method comprises administering to the patient about 60 mg to about 100 mg of the compound of Formula (I) or pharmaceutically acceptable salt thereof once per day. In some embodiments, the method comprises administering to the patient about 70 mg to about 200 mg of the compound of Formula (I) or pharmaceutically acceptable salt thereof once per day. In some embodiments, the method comprises administering to the patient about 70 mg to about 150 mg of the compound of Formula (I) or pharmaceutically acceptable salt thereof once per day. In some embodiments, the method comprises administering to the patient about 70 mg to about 100 mg of the compound of Formula (I) or pharmaceutically acceptable salt thereof once per day. In some embodiments, the method comprises administering to the patient about 80 mg to about 200 mg of the compound of Formula (I) or pharmaceutically acceptable salt thereof once per day. In some embodiments, the method comprises administering to the patient about 80 mg to about 150 mg of the compound of Formula (I) or pharmaceutically acceptable salt thereof once per day. In some embodiments, the method comprises administering to the patient about 80 mg to about 100 mg of the compound of Formula (I) or pharmaceutically acceptable salt thereof once per day. In some embodiments, the method comprises administering to the patient about 90 mg to about 200 mg of the compound of Formula (I) or pharmaceutically acceptable salt thereof once per day. In some embodiments, the method comprises administering to the patient about 90 mg to about 150 mg of the compound of Formula (I) or pharmaceutically acceptable salt thereof once per day. In some embodiments, the method comprises administering to the patient about 90 mg to about 100 mg of the compound of Formula (I) or pharmaceutically acceptable salt thereof once per day. In some embodiments, the method comprises administering to the patient about 100 mg to about 200 mg of the compound of Formula (I) or pharmaceutically acceptable salt thereof once per day. In some embodiments, the method comprises administering to the patient about 100 mg to about 150 mg of the compound of Formula (I) or pharmaceutically acceptable salt thereof once per day.

In some embodiments, the method comprises administering to the patient about 50 mg of the compound of Formula (I) or pharmaceutically acceptable salt thereof once per day. In some embodiments, the method comprises administering to the patient about 55 mg of the compound of Formula (I) or pharmaceutically acceptable salt thereof once per day. In some embodiments, the method comprises administering to the patient about 60 mg of the compound of Formula (I) or pharmaceutically acceptable salt thereof once per day. In some embodiments, the method comprises administering to the patient about 65 mg of the compound of Formula (I) or pharmaceutically acceptable salt thereof once per day. In some embodiments, the method comprises administering to the patient about 70 mg of the compound of Formula (I) or pharmaceutically acceptable salt thereof once per day. In some embodiments, the method comprises administering to the patient about 75 mg of the compound of Formula (I) or pharmaceutically acceptable salt thereof once per day. In some embodiments, the method comprises administering to the patient about 80 mg of the compound of Formula (I) or pharmaceutically acceptable salt thereof once per day. In some embodiments, the method comprises administering to the patient about 85 mg of the compound of Formula (I) or pharmaceutically acceptable salt thereof once per day. In some embodiments, the method comprises administering to the patient about 90 mg of the compound of Formula (I) or pharmaceutically acceptable salt thereof once per day. In some embodiments, the method comprises administering to the patient about 95 mg of the compound of Formula (I) or pharmaceutically acceptable salt thereof once per day. In some embodiments, the method comprises administering to the patient about 100 mg of the compound of Formula (I) or pharmaceutically acceptable salt thereof once per day. In some embodiments, the method comprises administering to the patient about 105 mg of the compound of Formula (I) or pharmaceutically acceptable salt thereof once per day. In some embodiments, the method comprises administering to the patient about 110 mg of the compound of Formula (I) or pharmaceutically acceptable salt thereof once per day. In some embodiments, the method comprises administering to the patient about 115 mg of the compound of Formula (I) or pharmaceutically acceptable salt thereof once per day. In some embodiments, the method comprises administering to the patient about 120 mg of the compound of Formula (I) or pharmaceutically acceptable salt thereof once per day. In some embodiments, the method comprises administering to the patient about 125 mg of the compound of Formula (I) or pharmaceutically acceptable salt thereof once per day. In some embodiments, the method comprises administering to the patient about 130 mg of the compound of Formula (I) or pharmaceutically acceptable salt thereof once per day. In some embodiments, the method comprises administering to the patient about 135 mg of the compound of Formula (I) or pharmaceutically acceptable salt thereof once per day. In some embodiments, the method comprises administering to the patient about 140 mg of the compound of Formula (I) or pharmaceutically acceptable salt thereof once per day. In some embodiments, the method comprises administering to the patient about 145 mg of the compound of Formula (I) or pharmaceutically acceptable salt thereof once per day. In some embodiments, the method comprises administering to the patient about 150 mg of the compound of Formula (I) or pharmaceutically acceptable salt thereof once per day. In some embodiments, the method comprises administering to the patient about 155 mg of the compound of Formula (I) or pharmaceutically acceptable salt thereof once per day. In some embodiments, the method comprises administering to the patient about 160 mg of the compound of Formula (I) or pharmaceutically acceptable salt thereof once per day. In some embodiments, the method comprises administering to the patient about 165 mg of the compound of Formula (I) or pharmaceutically acceptable salt thereof once per day. In some embodiments, the method comprises administering to the patient about 170 mg of the compound of Formula (I) or pharmaceutically acceptable salt thereof once per day. In some embodiments, the method comprises administering to the patient about 175 mg of the compound of Formula (I) or pharmaceutically acceptable salt thereof once per day. In some embodiments, the method comprises administering to the patient about 180 mg of the compound of Formula (I) or pharmaceutically acceptable salt thereof once per day. In some embodiments, the method comprises administering to the patient about 185 mg of the compound of Formula (I) or pharmaceutically acceptable salt thereof once per day. In some embodiments, the method comprises administering to the patient about 190 mg of the compound of Formula (I) or pharmaceutically acceptable salt thereof once per day. In some embodiments, the method comprises administering to the patient about 195 mg of the compound of Formula (I) or pharmaceutically acceptable salt thereof once per day. In some embodiments, the method comprises administering to the patient about 200 mg of the compound of Formula (I) or pharmaceutically acceptable salt thereof once per day.

In some embodiments, the method comprises administering to the patient about 50 mg to about 100 mg of the compound of Formula (I) or pharmaceutically acceptable salt thereof twice per day. In some embodiments, the method comprises administering to the patient about 60 mg to about 100 mg of the compound of Formula (I) or pharmaceutically acceptable salt thereof twice per day. In some embodiments, the method comprises administering to the patient about 70 mg to about 100 mg of the compound of Formula (I) or pharmaceutically acceptable salt thereof twice per day. In some embodiments, the method comprises administering to the patient about 80 mg to about 100 mg of the compound of Formula (I) or pharmaceutically acceptable salt thereof twice per day. In some embodiments, the method comprises administering to the patient about 90 mg to about 100 mg of the compound of Formula (I) or pharmaceutically acceptable salt thereof twice per day.

In some embodiments, the method comprises administering to the patient about 50 mg of the compound of Formula (I) or pharmaceutically acceptable salt thereof twice per day. In some embodiments, the method comprises administering to the patient about 55 mg of the compound of Formula (I) or pharmaceutically acceptable salt thereof twice per day. In some embodiments, the method comprises administering to the patient about 60 mg of the compound of Formula (I) or pharmaceutically acceptable salt thereof twice per day. In some embodiments, the method comprises administering to the patient about 65 mg of the compound of Formula (I) or pharmaceutically acceptable salt thereof twice per day. In some embodiments, the method comprises administering to the patient about 70 mg of the compound of Formula (I) or pharmaceutically acceptable salt thereof twice per day. In some embodiments, the method comprises administering to the patient about 75 mg of the compound of Formula (I) or pharmaceutically acceptable salt thereof twice per day. In some embodiments, the method comprises administering to the patient about 80 mg of the compound of Formula (I) or pharmaceutically acceptable salt thereof twice per day. In some embodiments, the method comprises administering to the patient about 85 mg of the compound of Formula (I) or pharmaceutically acceptable salt thereof twice per day. In some embodiments, the method comprises administering to the patient about 90 mg of the compound of Formula (I) or pharmaceutically acceptable salt thereof twice per day. In some embodiments, the method comprises administering to the patient about 95 mg of the compound of Formula (I) or pharmaceutically acceptable salt thereof twice per day. In some embodiments, the method comprises administering to the patient about 100 mg of the compound of Formula (I) or pharmaceutically acceptable salt thereof twice per day.

The compound of Formula (I) or pharmaceutically acceptable salt thereof may, in another embodiment, be administered to the patient on an intermittent basis in the methods of achieving a stable disease condition in a patient having pancreatic ductal adenocarcinoma described herein. In some embodiments, the compound of Formula (I) or pharmaceutically acceptable salt thereof is administered the patient every other day. In some embodiments, the compound of Formula (I) or pharmaceutically acceptable salt thereof is administered the patient every other day. In some embodiments, the compound of Formula (I) or pharmaceutically acceptable salt thereof is administered the patient once per week. In some embodiments, the compound of Formula (I) or pharmaceutically acceptable salt thereof is administered the patient twice per week. In some embodiments, the compound of Formula (I) or pharmaceutically acceptable salt thereof is administered the patient three times per week.

In some embodiments, the method comprises administering to the patient about 50 mg to about 200 mg of the compound of Formula (I) or pharmaceutically acceptable salt thereof once a week. In some embodiments, the method comprises administering to the patient about 50 mg to about 150 mg of the compound of Formula (I) or pharmaceutically acceptable salt thereof once a week. In some embodiments, the method comprises administering to the patient about 50 mg to about 100 mg of the compound of Formula (I) or pharmaceutically acceptable salt thereof once a week. In some embodiments, the method comprises administering to the patient about 60 mg to about 200 mg of the compound of Formula (I) or pharmaceutically acceptable salt thereof once a week. In some embodiments, the method comprises administering to the patient about 60 mg to about 150 mg of the compound of Formula (I) or pharmaceutically acceptable salt thereof once a week. In some embodiments, the method comprises administering to the patient about 60 mg to about 100 mg of the compound of Formula (I) or pharmaceutically acceptable salt thereof once a week. In some embodiments, the method comprises administering to the patient about 70 mg to about 200 mg of the compound of Formula (I) or pharmaceutically acceptable salt thereof once a week. In some embodiments, the method comprises administering to the patient about 70 mg to about 150 mg of the compound of Formula (I) or pharmaceutically acceptable salt thereof once a week. In some embodiments, the method comprises administering to the patient about 70 mg to about 100 mg of the compound of Formula (I) or pharmaceutically acceptable salt thereof once a week. In some embodiments, the method comprises administering to the patient about 80 mg to about 200 mg of the compound of Formula (I) or pharmaceutically acceptable salt thereof once a week. In some embodiments, the method comprises administering to the patient about 80 mg to about 150 mg of the compound of Formula (I) or pharmaceutically acceptable salt thereof once a week. In some embodiments, the method comprises administering to the patient about 80 mg to about 100 mg of the compound of Formula (I) or pharmaceutically acceptable salt thereof once a week. In some embodiments, the method comprises administering to the patient about 90 mg to about 200 mg of the compound of Formula (I) or pharmaceutically acceptable salt thereof once a week. In some embodiments, the method comprises administering to the patient about 90 mg to about 150 mg of the compound of Formula (I) or pharmaceutically acceptable salt thereof once a week. In some embodiments, the method comprises administering to the patient about 90 mg to about 100 mg of the compound of Formula (I) or pharmaceutically acceptable salt thereof once a week. In some embodiments, the method comprises administering to the patient about 100 mg to about 200 mg of the compound of Formula (I) or pharmaceutically acceptable salt thereof once a week. In some embodiments, the method comprises administering to the patient about 100 mg to about 150 mg of the compound of Formula (I) or pharmaceutically acceptable salt thereof once a week.

In some embodiments, the method comprises administering to the patient about 50 mg of the compound of Formula (I) or pharmaceutically acceptable salt thereof once a week. In some embodiments, the method comprises administering to the patient about 55 mg of the compound of Formula (I) or pharmaceutically acceptable salt thereof once a week. In some embodiments, the method comprises administering to the patient about 60 mg of the compound of Formula (I) or pharmaceutically acceptable salt thereof once a week. In some embodiments, the method comprises administering to the patient about 65 mg of the compound of Formula (I) or pharmaceutically acceptable salt thereof once a week. In some embodiments, the method comprises administering to the patient about 70 mg of the compound of Formula (I) or pharmaceutically acceptable salt thereof once a week. In some embodiments, the method comprises administering to the patient about 75 mg of the compound of Formula (I) or pharmaceutically acceptable salt thereof once a week. In some embodiments, the method comprises administering to the patient about 80 mg of the compound of Formula (I) or pharmaceutically acceptable salt thereof once a week. In some embodiments, the method comprises administering to the patient about 85 mg of the compound of Formula (I) or pharmaceutically acceptable salt thereof once a week. In some embodiments, the method comprises administering to the patient about 90 mg of the compound of Formula (I) or pharmaceutically acceptable salt thereof once a week. In some embodiments, the method comprises administering to the patient about 95 mg of the compound of Formula (I) or pharmaceutically acceptable salt thereof once a week. In some embodiments, the method comprises administering to the patient about 100 mg of the compound of Formula (I) or pharmaceutically acceptable salt thereof once a week. In some embodiments, the method comprises administering to the patient about 105 mg of the compound of Formula (I) or pharmaceutically acceptable salt thereof once a week. In some embodiments, the method comprises administering to the patient about 110 mg of the compound of Formula (I) or pharmaceutically acceptable salt thereof once a week. In some embodiments, the method comprises administering to the patient about 115 mg of the compound of Formula (I) or pharmaceutically acceptable salt thereof once a week. In some embodiments, the method comprises administering to the patient about 120 mg of the compound of Formula (I) or pharmaceutically acceptable salt thereof once a week. In some embodiments, the method comprises administering to the patient about 125 mg of the compound of Formula (I) or pharmaceutically acceptable salt thereof once a week. In some embodiments, the method comprises administering to the patient about 130 mg of the compound of Formula (I) or pharmaceutically acceptable salt thereof once a week. In some embodiments, the method comprises administering to the patient about 135 mg of the compound of Formula (I) or pharmaceutically acceptable salt thereof once a week. In some embodiments, the method comprises administering to the patient about 140 mg of the compound of Formula (I) or pharmaceutically acceptable salt thereof once a week. In some embodiments, the method comprises administering to the patient about 145 mg of the compound of Formula (I) or pharmaceutically acceptable salt thereof once a week. In some embodiments, the method comprises administering to the patient about 150 mg of the compound of Formula (I) or pharmaceutically acceptable salt thereof once a week. In some embodiments, the method comprises administering to the patient about 155 mg of the compound of Formula (I) or pharmaceutically acceptable salt thereof once a week. In some embodiments, the method comprises administering to the patient about 160 mg of the compound of Formula (I) or pharmaceutically acceptable salt thereof once a week. In some embodiments, the method comprises administering to the patient about 165 mg of the compound of Formula (I) or pharmaceutically acceptable salt thereof once a week. In some embodiments, the method comprises administering to the patient about 170 mg of the compound of Formula (I) or pharmaceutically acceptable salt thereof once a week. In some embodiments, the method comprises administering to the patient about 175 mg of the compound of Formula (I) or pharmaceutically acceptable salt thereof once a week. In some embodiments, the method comprises administering to the patient about 180 mg of the compound of Formula (I) or pharmaceutically acceptable salt thereof once a week. In some embodiments, the method comprises administering to the patient about 185 mg of the compound of Formula (I) or pharmaceutically acceptable salt thereof once a week. In some embodiments, the method comprises administering to the patient about 190 mg of the compound of Formula (I) or pharmaceutically acceptable salt thereof once a week. In some embodiments, the method comprises administering to the patient about 195 mg of the compound of Formula (I) or pharmaceutically acceptable salt thereof once a week. In some embodiments, the method comprises administering to the patient about 200 mg of the compound of Formula (I) or pharmaceutically acceptable salt thereof once a week.

In some embodiments, the method comprises administering to the patient about 50 mg to about 200 mg of the compound of Formula (I) or pharmaceutically acceptable salt thereof twice a week. In some embodiments, the method comprises administering to the patient about 50 mg to about 150 mg of the compound of Formula (I) or pharmaceutically acceptable salt thereof twice a week. In some embodiments, the method comprises administering to the patient about 50 mg to about 100 mg of the compound of Formula (I) or pharmaceutically acceptable salt thereof twice a week. In some embodiments, the method comprises administering to the patient about 60 mg to about 200 mg of the compound of Formula (I) or pharmaceutically acceptable salt thereof twice a week. In some embodiments, the method comprises administering to the patient about 60 mg to about 150 mg of the compound of Formula (I) or pharmaceutically acceptable salt thereof twice a week. In some embodiments, the method comprises administering to the patient about 60 mg to about 100 mg of the compound of Formula (I) or pharmaceutically acceptable salt thereof twice a week. In some embodiments, the method comprises administering to the patient about 70 mg to about 200 mg of the compound of Formula (I) or pharmaceutically acceptable salt thereof twice a week. In some embodiments, the method comprises administering to the patient about 70 mg to about 150 mg of the compound of Formula (I) or pharmaceutically acceptable salt thereof twice a week. In some embodiments, the method comprises administering to the patient about 70 mg to about 100 mg of the compound of Formula (I) or pharmaceutically acceptable salt thereof twice a week. In some embodiments, the method comprises administering to the patient about 80 mg to about 200 mg of the compound of Formula (I) or pharmaceutically acceptable salt thereof twice a week. In some embodiments, the method comprises administering to the patient about 80 mg to about 150 mg of the compound of Formula (I) or pharmaceutically acceptable salt thereof twice a week. In some embodiments, the method comprises administering to the patient about 80 mg to about 100 mg of the compound of Formula (I) or pharmaceutically acceptable salt thereof twice a week. In some embodiments, the method comprises administering to the patient about 90 mg to about 200 mg of the compound of Formula (I) or pharmaceutically acceptable salt thereof twice a week. In some embodiments, the method comprises administering to the patient about 90 mg to about 150 mg of the compound of Formula (I) or pharmaceutically acceptable salt thereof twice a week. In some embodiments, the method comprises administering to the patient about 90 mg to about 100 mg of the compound of Formula (I) or pharmaceutically acceptable salt thereof twice a week. In some embodiments, the method comprises administering to the patient about 100 mg to about 200 mg of the compound of Formula (I) or pharmaceutically acceptable salt thereof twice a week. In some embodiments, the method comprises administering to the patient about 100 mg to about 150 mg of the compound of Formula (I) or pharmaceutically acceptable salt thereof twice a week.

In some embodiments, the method comprises administering to the patient about 50 mg of the compound of Formula (I) or pharmaceutically acceptable salt thereof twice a week. In some embodiments, the method comprises administering to the patient about 55 mg of the compound of Formula (I) or pharmaceutically acceptable salt thereof twice a week. In some embodiments, the method comprises administering to the patient about 60 mg of the compound of Formula (I) or pharmaceutically acceptable salt thereof twice a week. In some embodiments, the method comprises administering to the patient about 65 mg of the compound of Formula (I) or pharmaceutically acceptable salt thereof twice a week. In some embodiments, the method comprises administering to the patient about 70 mg of the compound of Formula (I) or pharmaceutically acceptable salt thereof twice a week. In some embodiments, the method comprises administering to the patient about 75 mg of the compound of Formula (I) or pharmaceutically acceptable salt thereof twice a week. In some embodiments, the method comprises administering to the patient about 80 mg of the compound of Formula (I) or pharmaceutically acceptable salt thereof twice a week. In some embodiments, the method comprises administering to the patient about 85 mg of the compound of Formula (I) or pharmaceutically acceptable salt thereof twice a week. In some embodiments, the method comprises administering to the patient about 90 mg of the compound of Formula (I) or pharmaceutically acceptable salt thereof twice a week. In some embodiments, the method comprises administering to the patient about 95 mg of the compound of Formula (I) or pharmaceutically acceptable salt thereof twice a week. In some embodiments, the method comprises administering to the patient about 100 mg of the compound of Formula (I) or pharmaceutically acceptable salt thereof twice a week. In some embodiments, the method comprises administering to the patient about 105 mg of the compound of Formula (I) or pharmaceutically acceptable salt thereof twice a week. In some embodiments, the method comprises administering to the patient about 110 mg of the compound of Formula (I) or pharmaceutically acceptable salt thereof twice a week. In some embodiments, the method comprises administering to the patient about 115 mg of the compound of Formula (I) or pharmaceutically acceptable salt thereof twice a week. In some embodiments, the method comprises administering to the patient about 120 mg of the compound of Formula (I) or pharmaceutically acceptable salt thereof twice a week. In some embodiments, the method comprises administering to the patient about 125 mg of the compound of Formula (I) or pharmaceutically acceptable salt thereof twice a week. In some embodiments, the method comprises administering to the patient about 130 mg of the compound of Formula (I) or pharmaceutically acceptable salt thereof twice a week. In some embodiments, the method comprises administering to the patient about 135 mg of the compound of Formula (I) or pharmaceutically acceptable salt thereof twice a week. In some embodiments, the method comprises administering to the patient about 140 mg of the compound of Formula (I) or pharmaceutically acceptable salt thereof twice a week. In some embodiments, the method comprises administering to the patient about 145 mg of the compound of Formula (I) or pharmaceutically acceptable salt thereof twice a week. In some embodiments, the method comprises administering to the patient about 150 mg of the compound of Formula (I) or pharmaceutically acceptable salt thereof twice a week. In some embodiments, the method comprises administering to the patient about 155 mg of the compound of Formula (I) or pharmaceutically acceptable salt thereof twice a week. In some embodiments, the method comprises administering to the patient about 160 mg of the compound of Formula (I) or pharmaceutically acceptable salt thereof twice a week. In some embodiments, the method comprises administering to the patient about 165 mg of the compound of Formula (I) or pharmaceutically acceptable salt thereof twice a week. In some embodiments, the method comprises administering to the patient about 170 mg of the compound of Formula (I) or pharmaceutically acceptable salt thereof twice a week. In some embodiments, the method comprises administering to the patient about 175 mg of the compound of Formula (I) or pharmaceutically acceptable salt thereof twice a week. In some embodiments, the method comprises administering to the patient about 180 mg of the compound of Formula (I) or pharmaceutically acceptable salt thereof twice a week. In some embodiments, the method comprises administering to the patient about 185 mg of the compound of Formula (I) or pharmaceutically acceptable salt thereof twice a week. In some embodiments, the method comprises administering to the patient about 190 mg of the compound of Formula (I) or pharmaceutically acceptable salt thereof twice a week. In some embodiments, the method comprises administering to the patient about 195 mg of the compound of Formula (I) or pharmaceutically acceptable salt thereof twice a week. In some embodiments, the method comprises administering to the patient about 200 mg of the compound of Formula (I) or pharmaceutically acceptable salt thereof twice a week.

In some embodiments, the method comprises administering to the patient about 50 mg to about 200 mg of the compound of Formula (I) or pharmaceutically acceptable salt thereof three times a week. In some embodiments, the method comprises administering to the patient about 50 mg to about 150 mg of the compound of Formula (I) or pharmaceutically acceptable salt thereof three times a week. In some embodiments, the method comprises administering to the patient about 50 mg to about 100 mg of the compound of Formula (I) or pharmaceutically acceptable salt thereof three times a week. In some embodiments, the method comprises administering to the patient about 60 mg to about 200 mg of the compound of Formula (I) or pharmaceutically acceptable salt thereof three times a week. In some embodiments, the method comprises administering to the patient about 60 mg to about 150 mg of the compound of Formula (I) or pharmaceutically acceptable salt thereof three times a week. In some embodiments, the method comprises administering to the patient about 60 mg to about 100 mg of the compound of Formula (I) or pharmaceutically acceptable salt thereof three times a week. In some embodiments, the method comprises administering to the patient about 70 mg to about 200 mg of the compound of Formula (I) or pharmaceutically acceptable salt thereof three times a week. In some embodiments, the method comprises administering to the patient about 70 mg to about 150 mg of the compound of Formula (I) or pharmaceutically acceptable salt thereof three times a week. In some embodiments, the method comprises administering to the patient about 70 mg to about 100 mg of the compound of Formula (I) or pharmaceutically acceptable salt thereof three times a week. In some embodiments, the method comprises administering to the patient about 80 mg to about 200 mg of the compound of Formula (I) or pharmaceutically acceptable salt thereof three times a week. In some embodiments, the method comprises administering to the patient about 80 mg to about 150 mg of the compound of Formula (I) or pharmaceutically acceptable salt thereof three times a week. In some embodiments, the method comprises administering to the patient about 80 mg to about 100 mg of the compound of Formula (I) or pharmaceutically acceptable salt thereof three times a week. In some embodiments, the method comprises administering to the patient about 90 mg to about 200 mg of the compound of Formula (I) or pharmaceutically acceptable salt thereof three times a week. In some embodiments, the method comprises administering to the patient about 90 mg to about 150 mg of the compound of Formula (I) or pharmaceutically acceptable salt thereof three times a week. In some embodiments, the method comprises administering to the patient about 90 mg to about 100 mg of the compound of Formula (I) or pharmaceutically acceptable salt thereof three times a week. In some embodiments, the method comprises administering to the patient about 100 mg to about 200 mg of the compound of Formula (I) or pharmaceutically acceptable salt thereof three times a week. In some embodiments, the method comprises administering to the patient about 100 mg to about 150 mg of the compound of Formula (I) or pharmaceutically acceptable salt thereof three times a week.

In some embodiments, the method comprises administering to the patient about 50 mg of the compound of Formula (I) or pharmaceutically acceptable salt thereof three times a week. In some embodiments, the method comprises administering to the patient about 55 mg of the compound of Formula (I) or pharmaceutically acceptable salt thereof three times a week. In some embodiments, the method comprises administering to the patient about 60 mg of the compound of Formula (I) or pharmaceutically acceptable salt thereof three times a week. In some embodiments, the method comprises administering to the patient about 65 mg of the compound of Formula (I) or pharmaceutically acceptable salt thereof three times a week. In some embodiments, the method comprises administering to the patient about 70 mg of the compound of Formula (I) or pharmaceutically acceptable salt thereof three times a week. In some embodiments, the method comprises administering to the patient about 75 mg of the compound of Formula (I) or pharmaceutically acceptable salt thereof three times a week. In some embodiments, the method comprises administering to the patient about 80 mg of the compound of Formula (I) or pharmaceutically acceptable salt thereof three times a week. In some embodiments, the method comprises administering to the patient about 85 mg of the compound of Formula (I) or pharmaceutically acceptable salt thereof three times a week. In some embodiments, the method comprises administering to the patient about 90 mg of the compound of Formula (I) or pharmaceutically acceptable salt thereof three times a week. In some embodiments, the method comprises administering to the patient about 95 mg of the compound of Formula (I) or pharmaceutically acceptable salt thereof three times a week. In some embodiments, the method comprises administering to the patient about 100 mg of the compound of Formula (I) or pharmaceutically acceptable salt thereof three times a week. In some embodiments, the method comprises administering to the patient about 105 mg of the compound of Formula (I) or pharmaceutically acceptable salt thereof three times a week. In some embodiments, the method comprises administering to the patient about 110 mg of the compound of Formula (I) or pharmaceutically acceptable salt thereof three times a week. In some embodiments, the method comprises administering to the patient about 115 mg of the compound of Formula (I) or pharmaceutically acceptable salt thereof three times a week. In some embodiments, the method comprises administering to the patient about 120 mg of the compound of Formula (I) or pharmaceutically acceptable salt thereof three times a week. In some embodiments, the method comprises administering to the patient about 125 mg of the compound of Formula (I) or pharmaceutically acceptable salt thereof three times a week. In some embodiments, the method comprises administering to the patient about 130 mg of the compound of Formula (I) or pharmaceutically acceptable salt thereof three times a week. In some embodiments, the method comprises administering to the patient about 135 mg of the compound of Formula (I) or pharmaceutically acceptable salt thereof three times a week. In some embodiments, the method comprises administering to the patient about 140 mg of the compound of Formula (I) or pharmaceutically acceptable salt thereof three times a week. In some embodiments, the method comprises administering to the patient about 145 mg of the compound of Formula (I) or pharmaceutically acceptable salt thereof three times a week. In some embodiments, the method comprises administering to the patient about 150 mg of the compound of Formula (I) or pharmaceutically acceptable salt thereof three times a week. In some embodiments, the method comprises administering to the patient about 155 mg of the compound of Formula (I) or pharmaceutically acceptable salt thereof three times a week. In some embodiments, the method comprises administering to the patient about 160 mg of the compound of Formula (I) or pharmaceutically acceptable salt thereof three times a week. In some embodiments, the method comprises administering to the patient about 165 mg of the compound of Formula (I) or pharmaceutically acceptable salt thereof three times a week. In some embodiments, the method comprises administering to the patient about 170 mg of the compound of Formula (I) or pharmaceutically acceptable salt thereof three times a week. In some embodiments, the method comprises administering to the patient about 175 mg of the compound of Formula (I) or pharmaceutically acceptable salt thereof three times a week. In some embodiments, the method comprises administering to the patient about 180 mg of the compound of Formula (I) or pharmaceutically acceptable salt thereof three times a week. In some embodiments, the method comprises administering to the patient about 185 mg of the compound of Formula (I) or pharmaceutically acceptable salt thereof three times a week. In some embodiments, the method comprises administering to the patient about 190 mg of the compound of Formula (I) or pharmaceutically acceptable salt thereof three times a week. In some embodiments, the method comprises administering to the patient about 195 mg of the compound of Formula (I) or pharmaceutically acceptable salt thereof three times a week. In some embodiments, the method comprises administering to the patient about 200 mg of the compound of Formula (I) or pharmaceutically acceptable salt thereof three times a week.

In some embodiments, the method comprises administering to the patient about 50 mg to about 200 mg of the compound of Formula (I) or pharmaceutically acceptable salt thereof every other day. In some embodiments, the method comprises administering to the patient about 50 mg to about 150 mg of the compound of Formula (I) or pharmaceutically acceptable salt thereof every other day. In some embodiments, the method comprises administering to the patient about 50 mg to about 100 mg of the compound of Formula (I) or pharmaceutically acceptable salt thereof every other day. In some embodiments, the method comprises administering to the patient about 60 mg to about 200 mg of the compound of Formula (I) or pharmaceutically acceptable salt thereof every other day. In some embodiments, the method comprises administering to the patient about 60 mg to about 150 mg of the compound of Formula (I) or pharmaceutically acceptable salt thereof every other day. In some embodiments, the method comprises administering to the patient about 60 mg to about 100 mg of the compound of Formula (I) or pharmaceutically acceptable salt thereof every other day. In some embodiments, the method comprises administering to the patient about 70 mg to about 200 mg of the compound of Formula (I) or pharmaceutically acceptable salt thereof every other day. In some embodiments, the method comprises administering to the patient about 70 mg to about 150 mg of the compound of Formula (I) or pharmaceutically acceptable salt thereof every other day. In some embodiments, the method comprises administering to the patient about 70 mg to about 100 mg of the compound of Formula (I) or pharmaceutically acceptable salt thereof every other day. In some embodiments, the method comprises administering to the patient about 80 mg to about 200 mg of the compound of Formula (I) or pharmaceutically acceptable salt thereof every other day. In some embodiments, the method comprises administering to the patient about 80 mg to about 150 mg of the compound of Formula (I) or pharmaceutically acceptable salt thereof every other day. In some embodiments, the method comprises administering to the patient about 80 mg to about 100 mg of the compound of Formula (I) or pharmaceutically acceptable salt thereof every other day. In some embodiments, the method comprises administering to the patient about 90 mg to about 200 mg of the compound of Formula (I) or pharmaceutically acceptable salt thereof every other day. In some embodiments, the method comprises administering to the patient about 90 mg to about 150 mg of the compound of Formula (I) or pharmaceutically acceptable salt thereof every other day. In some embodiments, the method comprises administering to the patient about 90 mg to about 100 mg of the compound of Formula (I) or pharmaceutically acceptable salt thereof every other day. In some embodiments, the method comprises administering to the patient about 100 mg to about 200 mg of the compound of Formula (I) or pharmaceutically acceptable salt thereof every other day. In some embodiments, the method comprises administering to the patient about 100 mg to about 150 mg of the compound of Formula (I) or pharmaceutically acceptable salt thereof every other day.

In some embodiments, the method comprises administering to the patient about 50 mg of the compound of Formula (I) or pharmaceutically acceptable salt thereof every other day. In some embodiments, the method comprises administering to the patient about 55 mg of the compound of Formula (I) or pharmaceutically acceptable salt thereof every other day. In some embodiments, the method comprises administering to the patient about 60 mg of the compound of Formula (I) or pharmaceutically acceptable salt thereof every other day. In some embodiments, the method comprises administering to the patient about 65 mg of the compound of Formula (I) or pharmaceutically acceptable salt thereof every other day. In some embodiments, the method comprises administering to the patient about 70 mg of the compound of Formula (I) or pharmaceutically acceptable salt thereof every other day. In some embodiments, the method comprises administering to the patient about 75 mg of the compound of Formula (I) or pharmaceutically acceptable salt thereof every other day. In some embodiments, the method comprises administering to the patient about 80 mg of the compound of Formula (I) or pharmaceutically acceptable salt thereof every other day. In some embodiments, the method comprises administering to the patient about 85 mg of the compound of Formula (I) or pharmaceutically acceptable salt thereof every other day. In some embodiments, the method comprises administering to the patient about 90 mg of the compound of Formula (I) or pharmaceutically acceptable salt thereof every other day. In some embodiments, the method comprises administering to the patient about 95 mg of the compound of Formula (I) or pharmaceutically acceptable salt thereof every other day. In some embodiments, the method comprises administering to the patient about 100 mg of the compound of Formula (I) or pharmaceutically acceptable salt thereof every other day. In some embodiments, the method comprises administering to the patient about 105 mg of the compound of Formula (I) or pharmaceutically acceptable salt thereof every other day. In some embodiments, the method comprises administering to the patient about 110 mg of the compound of Formula (I) or pharmaceutically acceptable salt thereof every other day. In some embodiments, the method comprises administering to the patient about 115 mg of the compound embodiments, the method comprises administering to the patient about 120 mg of the compound of Formula (I) or pharmaceutically acceptable salt thereof every other day. In some embodiments, the method comprises administering to the patient about 125 mg of the compound of Formula (I) or pharmaceutically acceptable salt thereof every other day. In some embodiments, the method comprises administering to the patient about 130 mg of the compound of Formula (I) or pharmaceutically acceptable salt thereof every other day. In some embodiments, the method comprises administering to the patient about 135 mg of the compound of Formula (I) or pharmaceutically acceptable salt thereof every other day. In some embodiments, the method comprises administering to the patient about 140 mg of the compound of Formula (I) or pharmaceutically acceptable salt thereof every other day. In some embodiments, the method comprises administering to the patient about 145 mg of the compound of Formula (I) or pharmaceutically acceptable salt thereof every other day. In some embodiments, the method comprises administering to the patient about 150 mg of the compound of Formula (I) or pharmaceutically acceptable salt thereof every other day. In some embodiments, the method comprises administering to the patient about 155 mg of the compound of Formula (I) or pharmaceutically acceptable salt thereof every other day. In some embodiments, the method comprises administering to the patient about 160 mg of the compound of Formula (I) or pharmaceutically acceptable salt thereof every other day. In some embodiments, the method comprises administering to the patient about 165 mg of the compound of Formula (I) or pharmaceutically acceptable salt thereof every other day. In some embodiments, the method comprises administering to the patient about 170 mg of the compound of Formula (I) or pharmaceutically acceptable salt thereof every other day. In some embodiments, the method comprises administering to the patient about 175 mg of the compound of Formula (I) or pharmaceutically acceptable salt thereof every other day. In some embodiments, the method comprises administering to the patient about 180 mg of the compound of Formula (I) or pharmaceutically acceptable salt thereof every other day. In some embodiments, the method comprises administering to the patient about 185 mg of the compound of Formula (I) or pharmaceutically acceptable salt thereof every other day. In some embodiments, the method comprises administering to the patient about 190 mg of the compound of Formula (I) or pharmaceutically acceptable salt thereof every other day. In some embodiments, the method comprises administering to the patient about 195 mg of the compound of Formula (I) or pharmaceutically acceptable salt thereof every other day. In some embodiments, the method comprises administering to the patient about 200 mg of the compound of Formula (I) or pharmaceutically acceptable salt thereof every other day.

In some embodiments, the combination agent is intravenously administered to the patient in need thereof. The one or more combination agents administered to a patient in the methods of achieving a stable disease condition in patients having pancreatic ductal adenocarcinoma described herein can be dosed to achieve various concentrations of the combination agents in the patient's plasma, which can be quantified by the area under the curve (AUC) for each combination agent administered, e.g., intravenously administered. In some embodiments, the combination agent, (e.g., carboplatin) is intravenously administered to the patient in an amount sufficient to provide an AUC of about 5 to about 7 (e.g., an AUC of about 5 or about 6). In some embodiments, the method comprises reducing each of the amount of the compound of Formula (I) or pharmaceutically acceptable salt thereof and the combination agent administered to the patient after one or more months of administering to the patient in need thereof.

In some embodiments, the paclitaxel is intravenously administered to the patient, e.g., by intravenous infusion. The intravenously administered paclitaxel can be administered to a patient at various dose levels. In some embodiments, the paclitaxel is administered to the patient at about 50 mg/m² to about 150 mg/m². In some embodiments, the paclitaxel is administered to the patient at about 50 mg/m² to about 100 mg/m². In some embodiments, the paclitaxel is administered to the patient at about 60 mg/m² to about 80 mg/m². In some embodiments, the paclitaxel is administered to the patient at about 60 mg/m². In some embodiments, the paclitaxel is administered to the patient at about 80 mg/m². In some embodiments, the paclitaxel is administered to the patient at about 135 mg/m². In some embodiments, the paclitaxel is administered to the patient at about 175 mg/m².

Furthermore, the paclitaxel can be administered to the patient at various frequencies. In some embodiments, the paclitaxel is administered to the patient once per week (e.g., at about 60 mg/m² once per week or about 80 mg/m² once per week). In some embodiments, the paclitaxel is administered to the patient once every three weeks (e.g., at about 135 mg/m² once every three weeks or about 175 mg/m² once every three weeks). In some embodiments, the paclitaxel is administered to the patient by intravenous infusion once per week. In some embodiments, the paclitaxel is administered to the patient by intravenous infusion once per week for three consecutive weeks. In some embodiments, the method comprises administering paclitaxel to the patient by intravenous infusion for more than one cycle, wherein each cycle comprises administering paclitaxel to the patient by intravenous infusion once per week for three consecutive weeks (e.g., once per week for three consecutive weeks at about 60 mg/m² or about 80 mg/m²). In some embodiments, the method comprises not administering paclitaxel to the patient for at least one week between each cycle.

In some embodiments, the paclitaxel is administered to the patient by infusion once every three weeks. In some embodiments, the paclitaxel is administered to the patient for about 60 minutes once per week. In some embodiments, the paclitaxel is administered to the patient for about 3 hours once per week. In some embodiments, the paclitaxel is administered to the patient for about 60 minutes once every three weeks. In some embodiments, the paclitaxel is administered to the patient for about 3 hours once every three weeks. In some embodiments, the paclitaxel is administered to the patient by intravenous infusion for about 60 minutes once per week. In some embodiments, the paclitaxel is administered to the patient by infusion for about 3 hours once per week. In some embodiments, the paclitaxel is administered to the patient by intravenous infusion for about 60 minutes once every three weeks. In some embodiments, the paclitaxel is administered to the patient by infusion for about 3 hours once every three weeks. In some embodiments, the paclitaxel is continuously administered to the patient for about 60 minutes once per week. In some embodiments, the paclitaxel is continuously administered to the patient for about 3 hours once per week. In some embodiments, the paclitaxel is continuously administered to the patient for about 60 minutes once every three weeks. In some embodiments, the paclitaxel is continuously administered to the patient for about 3 hours once every three weeks. In some embodiments, the paclitaxel is continuously administered to the patient by infusion for about 60 minutes once per week. In some embodiments, the paclitaxel is continuously administered to the patient by intravenous infusion for about 3 hours once per week. In some embodiments, the paclitaxel is continuously administered to the patient by infusion for about 60 minutes once every three weeks. In some embodiments, the paclitaxel is continuously administered to the patient by intravenous infusion for about 3 hours once every three weeks.

The method of achieving a stable disease condition in patients having pancreatic ductal adenocarcinoma described herein can further comprise reducing each of the amount of the compound of Formula (I) or pharmaceutically acceptable salt thereof and the combination agent administered to the patient after one or more months of administering said compound of Formula (I) or pharmaceutically acceptable salt thereof and said combination agent to the patient in need thereof.

In embodiments of the methods of achieving at least a stable disease condition in a patient having an pancreatic ductal adenocarcinoma described herein, the disclosure also contemplates administration of the compound of Formula (I) or a pharmaceutically acceptable salt to a patient in need thereof prior to (neo-adjuvant) or after (adjuvant) surgery (e.g., surgical treatment of a solid tumor). In some embodiments, the compound of Formula (I) or a pharmaceutically acceptable salt is administered as an adjuvant. In some embodiments, the compound of Formula (I) or a pharmaceutically acceptable salt is administered as a neo-adjuvant. In some embodiments, the compound of Formula (I) or a pharmaceutically acceptable salt is administered as a neo-adjuvant and an adjuvant. In some embodiments, the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 100 years. In some embodiments, administration of the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 5 years. In some embodiments, administration of the compound of Formula (I) or a pharmaceutically acceptable salt is administered to a patient in need thereof as a neo-adjuvant for a period of 3 months to 6 months, followed by administration of the compound of Formula (I) or a pharmaceutically acceptable salt as an adjuvant for a period from 1 day to 5 years. In some embodiments, no administration of the compound of Formula (I) or a pharmaceutically acceptable salt occurs prior to surgery. In some embodiments, the compound of Formula (I) or a pharmaceutically acceptable salt is administered as an adjuvant for a period from 1 day to 100 years. In some embodiments, the compound of Formula (I) or a pharmaceutically acceptable salt is administered as an adjuvant for a period from 1 day to 5 years.

### Lung Cancer

Also provided herein is a method of treating lung cancer in a patient in need thereof, comprising administering to the patient an effective amount of the compound of Formula (I)

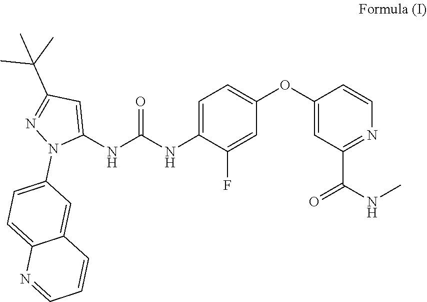 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In one embodiment, provided herein is a method of treating lung cancer in a patient in need thereof, comprising administering to the patient about 50 mg to about 200 mg of the compound of Formula (I)

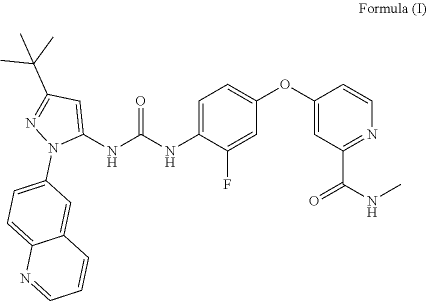 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof per day.

Contemplated lung cancers include non-small cell lung cancer (e.g., advanced non-small cell lung cancer) and small-cell lung cancer.

In embodiments of the methods of treating an lung cancer described herein, the compound of Formula (I) or pharmaceutically acceptable salt thereof can be orally administered to the patient in need thereof. In certain embodiments, the compound of Formula (I) or pharmaceutically acceptable salt thereof is administered to the patient daily. In some embodiments, the method comprises administering to the patient about 50 mg to about 150 mg of the compound of Formula (I) or pharmaceutically acceptable salt thereof per day. In some embodiments, the method comprises administering to the patient about 50 mg to about 100 mg of the compound of Formula (I) or pharmaceutically acceptable salt thereof per day. In some embodiments, the method comprises administering to the patient about 60 mg to about 200 mg of the compound of Formula (I) or pharmaceutically acceptable salt thereof per day. In some embodiments, the method comprises administering to the patient about 60 mg to about 150 mg of the compound of Formula (I) or pharmaceutically acceptable salt thereof per day. In some embodiments, the method comprises administering to the patient about 60 mg to about 100 mg of the compound of Formula (I) or pharmaceutically acceptable salt thereof per day. In some embodiments, the method comprises administering to the patient about 70 mg to about 200 mg of the compound of Formula (I) or pharmaceutically acceptable salt thereof per day. In some embodiments, the method comprises administering to the patient about 70 mg to about 150 mg of the compound of Formula (I) or pharmaceutically acceptable salt thereof per day. In some embodiments, the method comprises administering to the patient about 70 mg to about 100 mg of the compound of Formula (I) or pharmaceutically acceptable salt thereof per day. In some embodiments, the method comprises administering to the patient about 80 mg to about 200 mg of the compound of Formula (I) or pharmaceutically acceptable salt thereof per day. In some embodiments, the method comprises administering to the patient about 80 mg to about 150 mg of the compound of Formula (I) or pharmaceutically acceptable salt thereof per day. In some embodiments, the method comprises administering to the patient about 80 mg to about 100 mg of the compound of Formula (I) or pharmaceutically acceptable salt thereof per day. In some embodiments, the method comprises administering to the patient about 90 mg to about 200 mg of the compound of Formula (I) or pharmaceutically acceptable salt thereof per day. In some embodiments, the method comprises administering to the patient about 90 mg to about 150 mg of the compound of Formula (I) or pharmaceutically acceptable salt thereof per day. In some embodiments, the method comprises administering to the patient about 90 mg to about 100 mg of the compound of Formula (I) or pharmaceutically acceptable salt thereof per day. In some embodiments, the method comprises administering to the patient about 100 mg to about 200 mg of the compound of Formula (I) or pharmaceutically acceptable salt thereof per day. In some embodiments, the method comprises administering to the patient about 100 mg to about 150 mg of the compound of Formula (I) or pharmaceutically acceptable salt thereof per day.

In some embodiments, the method comprises administering to the patient about 50 mg of the compound of Formula (I) or pharmaceutically acceptable salt thereof per day. In some embodiments, the method comprises administering to the patient about 55 mg of the compound of Formula (I) or pharmaceutically acceptable salt thereof per day. In some embodiments, the method comprises administering to the patient about 60 mg of the compound of Formula (I) or pharmaceutically acceptable salt thereof per day. In some embodiments, the method comprises administering to the patient about 65 mg of the compound of Formula (I) or pharmaceutically acceptable salt thereof per day. In some embodiments, the method comprises administering to the patient about 70 mg of the compound of Formula (I) or pharmaceutically acceptable salt thereof per day. In some embodiments, the method comprises administering to the patient about 75 mg of the compound of Formula (I) or pharmaceutically acceptable salt thereof per day. In some embodiments, the method comprises administering to the patient about 80 mg of the compound of Formula (I) or pharmaceutically acceptable salt thereof per day. In some embodiments, the method comprises administering to the patient about 85 mg of the compound of Formula (I) or pharmaceutically acceptable salt thereof per day. In some embodiments, the method comprises administering to the patient about 90 mg of the compound of Formula (I) or pharmaceutically acceptable salt thereof per day. In some embodiments, the method comprises administering to the patient about 95 mg of the compound of Formula (I) or pharmaceutically acceptable salt thereof per day. In some embodiments, the method comprises administering to the patient about 100 mg of the compound of Formula (I) or pharmaceutically acceptable salt thereof per day. In some embodiments, the method comprises administering to the patient about 105 mg of the compound of Formula (I) or pharmaceutically acceptable salt thereof per day. In some embodiments, the method comprises administering to the patient about 110 mg of the compound of Formula (I) or pharmaceutically acceptable salt thereof per day. In some embodiments, the method comprises administering to the patient about 115 mg of the compound of Formula (I) or pharmaceutically acceptable salt thereof per day. In some embodiments, the method comprises administering to the patient about 120 mg of the compound of Formula (I) or pharmaceutically acceptable salt thereof per day. In some embodiments, the method comprises administering to the patient about 125 mg of the compound of Formula (I) or pharmaceutically acceptable salt thereof per day. In some embodiments, the method comprises administering to the patient about 130 mg of the compound of Formula (I) or pharmaceutically acceptable salt thereof per day. In some embodiments, the method comprises administering to the patient about 135 mg of the compound of Formula (I) or pharmaceutically acceptable salt thereof per day. In some embodiments, the method comprises administering to the patient about 140 mg of the compound of Formula (I) or pharmaceutically acceptable salt thereof per day. In some embodiments, the method comprises administering to the patient about 145 mg of the compound of Formula (I) or pharmaceutically acceptable salt thereof per day. In some embodiments, the method comprises administering to the patient about 150 mg of the compound of Formula (I) or pharmaceutically acceptable salt thereof per day. In some embodiments, the method comprises administering to the patient about 155 mg of the compound of Formula (I) or pharmaceutically acceptable salt thereof per day. In some embodiments, the method comprises administering to the patient about 160 mg of the compound of Formula (I) or pharmaceutically acceptable salt thereof per day. In some embodiments, the method comprises administering to the patient about 165 mg of the compound of Formula (I) or pharmaceutically acceptable salt thereof per day. In some embodiments, the method comprises administering to the patient about 170 mg of the compound of Formula (I) or pharmaceutically acceptable salt thereof per day. In some embodiments, the method comprises administering to the patient about 175 mg of the compound of Formula (I) or pharmaceutically acceptable salt thereof per day. In some embodiments, the method comprises administering to the patient about 180 mg of the compound of Formula (I) or pharmaceutically acceptable salt thereof per day. In some embodiments, the method comprises administering to the patient about 185 mg of the compound of Formula (I) or pharmaceutically acceptable salt thereof per day. In some embodiments, the method comprises administering to the patient about 190 mg of the compound of Formula (I) or pharmaceutically acceptable salt thereof per day. In some embodiments, the method comprises administering to the patient about 195 mg of the compound of Formula (I) or pharmaceutically acceptable salt thereof per day. In some embodiments, the method comprises administering to the patient about 200 mg of the compound of Formula (I) or pharmaceutically acceptable salt thereof per day.

In some embodiments, the method comprises administering to the patient about 50 mg to about 200 mg of the compound of Formula (I) or pharmaceutically acceptable salt thereof once per day. In some embodiments, the method comprises administering to the patient about 50 mg to about 150 mg of the compound of Formula (I) or pharmaceutically acceptable salt thereof once per day. In some embodiments, the method comprises administering to the patient about 50 mg to about 100 mg of the compound of Formula (I) or pharmaceutically acceptable salt thereof once per day. In some embodiments, the method comprises administering to the patient about 60 mg to about 200 mg of the compound of Formula (I) or pharmaceutically acceptable salt thereof once per day. In some embodiments, the method comprises administering to the patient about 60 mg to about 150 mg of the compound of Formula (I) or pharmaceutically acceptable salt thereof once per day. In some embodiments, the method comprises administering to the patient about 60 mg to about 100 mg of the compound of Formula (I) or pharmaceutically acceptable salt thereof once per day. In some embodiments, the method comprises administering to the patient about 70 mg to about 200 mg of the compound of Formula (I) or pharmaceutically acceptable salt thereof once per day. In some embodiments, the method comprises administering to the patient about 70 mg to about 150 mg of the compound of Formula (I) or pharmaceutically acceptable salt thereof once per day. In some embodiments, the method comprises administering to the patient about 70 mg to about 100 mg of the compound of Formula (I) or pharmaceutically acceptable salt thereof once per day. In some embodiments, the method comprises administering to the patient about 80 mg to about 200 mg of the compound of Formula (I) or pharmaceutically acceptable salt thereof once per day. In some embodiments, the method comprises administering to the patient about 80 mg to about 150 mg of the compound of Formula (I) or pharmaceutically acceptable salt thereof once per day. In some embodiments, the method comprises administering to the patient about 80 mg to about 100 mg of the compound of Formula (I) or pharmaceutically acceptable salt thereof once per day. In some embodiments, the method comprises administering to the patient about 90 mg to about 200 mg of the compound of Formula (I) or pharmaceutically acceptable salt thereof once per day. In some embodiments, the method comprises administering to the patient about 90 mg to about 150 mg of the compound of Formula (I) or pharmaceutically acceptable salt thereof once per day. In some embodiments, the method comprises administering to the patient about 90 mg to about 100 mg of the compound of Formula (I) or pharmaceutically acceptable salt thereof once per day. In some embodiments, the method comprises administering to the patient about 100 mg to about 200 mg of the compound of Formula (I) or pharmaceutically acceptable salt thereof once per day. In some embodiments, the method comprises administering to the patient about 100 mg to about 150 mg of the compound of Formula (I) or pharmaceutically acceptable salt thereof once per day.

In some embodiments, the method comprises administering to the patient about 50 mg of the compound of Formula (I) or pharmaceutically acceptable salt thereof once per day. In some embodiments, the method comprises administering to the patient about 55 mg of the compound of Formula (I) or pharmaceutically acceptable salt thereof once per day. In some embodiments, the method comprises administering to the patient about 60 mg of the compound of Formula (I) or pharmaceutically acceptable salt thereof once per day. In some embodiments, the method comprises administering to the patient about 65 mg of the compound of Formula (I) or pharmaceutically acceptable salt thereof once per day. In some embodiments, the method comprises administering to the patient about 70 mg of the compound of Formula (I) or pharmaceutically acceptable salt thereof once per day. In some embodiments, the method comprises administering to the patient about 75 mg of the compound of Formula (I) or pharmaceutically acceptable salt thereof once per day. In some embodiments, the method comprises administering to the patient about 80 mg of the compound of Formula (I) or pharmaceutically acceptable salt thereof once per day. In some embodiments, the method comprises administering to the patient about 85 mg of the compound of Formula (I) or pharmaceutically acceptable salt thereof once per day. In some embodiments, the method comprises administering to the patient about 90 mg of the compound of Formula (I) or pharmaceutically acceptable salt thereof once per day. In some embodiments, the method comprises administering to the patient about 95 mg of the compound of Formula (I) or pharmaceutically acceptable salt thereof once per day. In some embodiments, the method comprises administering to the patient about 100 mg of the compound of Formula (I) or pharmaceutically acceptable salt thereof once per day. In some embodiments, the method comprises administering to the patient about 105 mg of the compound of Formula (I) or pharmaceutically acceptable salt thereof once per day. In some embodiments, the method comprises administering to the patient about 110 mg of the compound of Formula (I) or pharmaceutically acceptable salt thereof once per day. In some embodiments, the method comprises administering to the patient about 115 mg of the compound of Formula (I) or pharmaceutically acceptable salt thereof once per day. In some embodiments, the method comprises administering to the patient about 120 mg of the compound of Formula (I) or pharmaceutically acceptable salt thereof once per day. In some embodiments, the method comprises administering to the patient about 125 mg of the compound of Formula (I) or pharmaceutically acceptable salt thereof once per day. In some embodiments, the method comprises administering to the patient about 130 mg of the compound of Formula (I) or pharmaceutically acceptable salt thereof once per day. In some embodiments, the method comprises administering to the patient about 135 mg of the compound of Formula (I) or pharmaceutically acceptable salt thereof once per day. In some embodiments, the method comprises administering to the patient about 140 mg of the compound of Formula (I) or pharmaceutically acceptable salt thereof once per day. In some embodiments, the method comprises administering to the patient about 145 mg of the compound of Formula (I) or pharmaceutically acceptable salt thereof once per day. In some embodiments, the method comprises administering to the patient about 150 mg of the compound of Formula (I) or pharmaceutically acceptable salt thereof once per day. In some embodiments, the method comprises administering to the patient about 155 mg of the compound of Formula (I) or pharmaceutically acceptable salt thereof once per day. In some embodiments, the method comprises administering to the patient about 160 mg of the compound of Formula (I) or pharmaceutically acceptable salt thereof once per day. In some embodiments, the method comprises administering to the patient about 165 mg of the compound of Formula (I) or pharmaceutically acceptable salt thereof once per day. In some embodiments, the method comprises administering to the patient about 170 mg of the compound of Formula (I) or pharmaceutically acceptable salt thereof once per day. In some embodiments, the method comprises administering to the patient about 175 mg of the compound of Formula (I) or pharmaceutically acceptable salt thereof once per day. In some embodiments, the method comprises administering to the patient about 180 mg of the compound of Formula (I) or pharmaceutically acceptable salt thereof once per day. In some embodiments, the method comprises administering to the patient about 185 mg of the compound of Formula (I) or pharmaceutically acceptable salt thereof once per day. In some embodiments, the method comprises administering to the patient about 190 mg of the compound of Formula (I) or pharmaceutically acceptable salt thereof once per day. In some embodiments, the method comprises administering to the patient about 195 mg of the compound of Formula (I) or pharmaceutically acceptable salt thereof once per day. In some embodiments, the method comprises administering to the patient about 200 mg of the compound of Formula (I) or pharmaceutically acceptable salt thereof once per day.

In some embodiments, the method comprises administering to the patient about 50 mg to about 100 mg of the compound of Formula (I) or pharmaceutically acceptable salt thereof twice per day. In some embodiments, the method comprises administering to the patient about 60 mg to about 100 mg of the compound of Formula (I) or pharmaceutically acceptable salt thereof twice per day. In some embodiments, the method comprises administering to the patient about 70 mg to about 100 mg of the compound of Formula (I) or pharmaceutically acceptable salt thereof twice per day. In some embodiments, the method comprises administering to the patient about 80 mg to about 100 mg of the compound of Formula (I) or pharmaceutically acceptable salt thereof twice per day. In some embodiments, the method comprises administering to the patient about 90 mg to about 100 mg of the compound of Formula (I) or pharmaceutically acceptable salt thereof twice per day.

In some embodiments, the method comprises administering to the patient about 50 mg of the compound of Formula (I) or pharmaceutically acceptable salt thereof twice per day. In some embodiments, the method comprises administering to the patient about 55 mg of the compound of Formula (I) or pharmaceutically acceptable salt thereof twice per day. In some embodiments, the method comprises administering to the patient about 60 mg of the compound of Formula (I) or pharmaceutically acceptable salt thereof twice per day. In some embodiments, the method comprises administering to the patient about 65 mg of the compound of Formula (I) or pharmaceutically acceptable salt thereof twice per day. In some embodiments, the method comprises administering to the patient about 70 mg of the compound of Formula (I) or pharmaceutically acceptable salt thereof twice per day. In some embodiments, the method comprises administering to the patient about 75 mg of the compound of Formula (I) or pharmaceutically acceptable salt thereof twice per day. In some embodiments, the method comprises administering to the patient about 80 mg of the compound of Formula (I) or pharmaceutically acceptable salt thereof twice per day. In some embodiments, the method comprises administering to the patient about 85 mg of the compound of Formula (I) or pharmaceutically acceptable salt thereof twice per day. In some embodiments, the method comprises administering to the patient about 90 mg of the compound of Formula (I) or pharmaceutically acceptable salt thereof twice per day. In some embodiments, the method comprises administering to the patient about 95 mg of the compound of Formula (I) or pharmaceutically acceptable salt thereof twice per day. In some embodiments, the method comprises administering to the patient about 100 mg of the compound of Formula (I) or pharmaceutically acceptable salt thereof twice per day.

The compound of Formula (I) or pharmaceutically acceptable salt thereof may, in another embodiment, be administered to the patient on an intermittent basis in the methods of treating lung cancer described herein. In some embodiments, the compound of Formula (I) or pharmaceutically acceptable salt thereof is administered the patient every other day. In some embodiments, the compound of Formula (I) or pharmaceutically acceptable salt thereof is administered the patient every other day. In some embodiments, the compound of Formula (I) or pharmaceutically acceptable salt thereof is administered the patient once per week. In some embodiments, the compound of Formula (I) or pharmaceutically acceptable salt thereof is administered the patient twice per week. In some embodiments, the compound of Formula (I) or pharmaceutically acceptable salt thereof is administered the patient three times per week.

In some embodiments, the method comprises administering to the patient about 50 mg to about 200 mg of the compound of Formula (I) or pharmaceutically acceptable salt thereof once a week. In some embodiments, the method comprises administering to the patient about 50 mg to about 150 mg of the compound of Formula (I) or pharmaceutically acceptable salt thereof once a week. In some embodiments, the method comprises administering to the patient about 50 mg to about 100 mg of the compound of Formula (I) or pharmaceutically acceptable salt thereof once a week. In some embodiments, the method comprises administering to the patient about 60 mg to about 200 mg of the compound of Formula (I) or pharmaceutically acceptable salt thereof once a week. In some embodiments, the method comprises administering to the patient about 60 mg to about 150 mg of the compound of Formula (I) or pharmaceutically acceptable salt thereof once a week. In some embodiments, the method comprises administering to the patient about 60 mg to about 100 mg of the compound of Formula (I) or pharmaceutically acceptable salt thereof once a week. In some embodiments, the method comprises administering to the patient about 70 mg to about 200 mg of the compound of Formula (I) or pharmaceutically acceptable salt thereof once a week. In some embodiments, the method comprises administering to the patient about 70 mg to about 150 mg of the compound of Formula (I) or pharmaceutically acceptable salt thereof once a week. In some embodiments, the method comprises administering to the patient about 70 mg to about 100 mg of the compound of Formula (I) or pharmaceutically acceptable salt thereof once a week. In some embodiments, the method comprises administering to the patient about 80 mg to about 200 mg of the compound of Formula (I) or pharmaceutically acceptable salt thereof once a week. In some embodiments, the method comprises administering to the patient about 80 mg to about 150 mg of the compound of Formula (I) or pharmaceutically acceptable salt thereof once a week. In some embodiments, the method comprises administering to the patient about 80 mg to about 100 mg of the compound of Formula (I) or pharmaceutically acceptable salt thereof once a week. In some embodiments, the method comprises administering to the patient about 90 mg to about 200 mg of the compound of Formula (I) or pharmaceutically acceptable salt thereof once a week. In some embodiments, the method comprises administering to the patient about 90 mg to about 150 mg of the compound of Formula (I) or pharmaceutically acceptable salt thereof once a week. In some embodiments, the method comprises administering to the patient about 90 mg to about 100 mg of the compound of Formula (I) or pharmaceutically acceptable salt thereof once a week. In some embodiments, the method comprises administering to the patient about 100 mg to about 200 mg of the compound of Formula (I) or pharmaceutically acceptable salt thereof once a week. In some embodiments, the method comprises administering to the patient about 100 mg to about 150 mg of the compound of Formula (I) or pharmaceutically acceptable salt thereof once a week.

In some embodiments, the method comprises administering to the patient about 50 mg of the compound of Formula (I) or pharmaceutically acceptable salt thereof once a week. In some embodiments, the method comprises administering to the patient about 55 mg of the compound of Formula (I) or pharmaceutically acceptable salt thereof once a week. In some embodiments, the method comprises administering to the patient about 60 mg of the compound of Formula (I) or pharmaceutically acceptable salt thereof once a week. In some embodiments, the method comprises administering to the patient about 65 mg of the compound of Formula (I) or pharmaceutically acceptable salt thereof once a week. In some embodiments, the method comprises administering to the patient about 70 mg of the compound of Formula (I) or pharmaceutically acceptable salt thereof once a week. In some embodiments, the method comprises administering to the patient about 75 mg of the compound of Formula (I) or pharmaceutically acceptable salt thereof once a week. In some embodiments, the method comprises administering to the patient about 80 mg of the compound of Formula (I) or pharmaceutically acceptable salt thereof once a week. In some embodiments, the method comprises administering to the patient about 85 mg of the compound of Formula (I) or pharmaceutically acceptable salt thereof once a week. In some embodiments, the method comprises administering to the patient about 90 mg of the compound of Formula (I) or pharmaceutically acceptable salt thereof once a week. In some embodiments, the method comprises administering to the patient about 95 mg of the compound of Formula (I) or pharmaceutically acceptable salt thereof once a week. In some embodiments, the method comprises administering to the patient about 100 mg of the compound of Formula (I) or pharmaceutically acceptable salt thereof once a week. In some embodiments, the method comprises administering to the patient about 105 mg of the compound of Formula (I) or pharmaceutically acceptable salt thereof once a week. In some embodiments, the method comprises administering to the patient about 110 mg of the compound of Formula (I) or pharmaceutically acceptable salt thereof once a week. In some embodiments, the method comprises administering to the patient about 115 mg of the compound of Formula (I) or pharmaceutically acceptable salt thereof once a week. In some embodiments, the method comprises administering to the patient about 120 mg of the compound of Formula (I) or pharmaceutically acceptable salt thereof once a week. In some embodiments, the method comprises administering to the patient about 125 mg of the compound of Formula (I) or pharmaceutically acceptable salt thereof once a week. In some embodiments, the method comprises administering to the patient about 130 mg of the compound of Formula (I) or pharmaceutically acceptable salt thereof once a week. In some embodiments, the method comprises administering to the patient about 135 mg of the compound of Formula (I) or pharmaceutically acceptable salt thereof once a week. In some embodiments, the method comprises administering to the patient about 140 mg of the compound of Formula (I) or pharmaceutically acceptable salt thereof once a week. In some embodiments, the method comprises administering to the patient about 145 mg of the compound of Formula (I) or pharmaceutically acceptable salt thereof once a week. In some embodiments, the method comprises administering to the patient about 150 mg of the compound of Formula (I) or pharmaceutically acceptable salt thereof once a week. In some embodiments, the method comprises administering to the patient about 155 mg of the compound of Formula (I) or pharmaceutically acceptable salt thereof once a week. In some embodiments, the method comprises administering to the patient about 160 mg of the compound of Formula (I) or pharmaceutically acceptable salt thereof once a week. In some embodiments, the method comprises administering to the patient about 165 mg of the compound of Formula (I) or pharmaceutically acceptable salt thereof once a week. In some embodiments, the method comprises administering to the patient about 170 mg of the compound of Formula (I) or pharmaceutically acceptable salt thereof once a week. In some embodiments, the method comprises administering to the patient about 175 mg of the compound of Formula (I) or pharmaceutically acceptable salt thereof once a week. In some embodiments, the method comprises administering to the patient about 180 mg of the compound of Formula (I) or pharmaceutically acceptable salt thereof once a week. In some embodiments, the method comprises administering to the patient about 185 mg of the compound of Formula (I) or pharmaceutically acceptable salt thereof once a week. In some embodiments, the method comprises administering to the patient about 190 mg of the compound of Formula (I) or pharmaceutically acceptable salt thereof once a week. In some embodiments, the method comprises administering to the patient about 195 mg of the compound of Formula (I) or pharmaceutically acceptable salt thereof once a week. In some embodiments, the method comprises administering to the patient about 200 mg of the compound of Formula (I) or pharmaceutically acceptable salt thereof once a week.

In some embodiments, the method comprises administering to the patient about 50 mg to about 200 mg of the compound of Formula (I) or pharmaceutically acceptable salt thereof twice a week. In some embodiments, the method comprises administering to the patient about 50 mg to about 150 mg of the compound of Formula (I) or pharmaceutically acceptable salt thereof twice a week. In some embodiments, the method comprises administering to the patient about 50 mg to about 100 mg of the compound of Formula (I) or pharmaceutically acceptable salt thereof twice a week. In some embodiments, the method comprises administering to the patient about 60 mg to about 200 mg of the compound of Formula (I) or pharmaceutically acceptable salt thereof twice a week. In some embodiments, the method comprises administering to the patient about 60 mg to about 150 mg of the compound of Formula (I) or pharmaceutically acceptable salt thereof twice a week. In some embodiments, the method comprises administering to the patient about 60 mg to about 100 mg of the compound of Formula (I) or pharmaceutically acceptable salt thereof twice a week. In some embodiments, the method comprises administering to the patient about 70 mg to about 200 mg of the compound of Formula (I) or pharmaceutically acceptable salt thereof twice a week. In some embodiments, the method comprises administering to the patient about 70 mg to about 150 mg of the compound of Formula (I) or pharmaceutically acceptable salt thereof twice a week. In some embodiments, the method comprises administering to the patient about 70 mg to about 100 mg of the compound of Formula (I) or pharmaceutically acceptable salt thereof twice a week. In some embodiments, the method comprises administering to the patient about 80 mg to about 200 mg of the compound of Formula (I) or pharmaceutically acceptable salt thereof twice a week. In some embodiments, the method comprises administering to the patient about 80 mg to about 150 mg of the compound of Formula (I) or pharmaceutically acceptable salt thereof twice a week. In some embodiments, the method comprises administering to the patient about 80 mg to about 100 mg of the compound of Formula (I) or pharmaceutically acceptable salt thereof twice a week. In some embodiments, the method comprises administering to the patient about 90 mg to about 200 mg of the compound of Formula (I) or pharmaceutically acceptable salt thereof twice a week. In some embodiments, the method comprises administering to the patient about 90 mg to about 150 mg of the compound of Formula (I) or pharmaceutically acceptable salt thereof twice a week. In some embodiments, the method comprises administering to the patient about 90 mg to about 100 mg of the compound of Formula (I) or pharmaceutically acceptable salt thereof twice a week. In some embodiments, the method comprises administering to the patient about 100 mg to about 200 mg of the compound of Formula (I) or pharmaceutically acceptable salt thereof twice a week. In some embodiments, the method comprises administering to the patient about 100 mg to about 150 mg of the compound of Formula (I) or pharmaceutically acceptable salt thereof twice a week.

In some embodiments, the method comprises administering to the patient about 50 mg of the compound of Formula (I) or pharmaceutically acceptable salt thereof twice a week. In some embodiments, the method comprises administering to the patient about 55 mg of the compound of Formula (I) or pharmaceutically acceptable salt thereof twice a week. In some embodiments, the method comprises administering to the patient about 60 mg of the compound of Formula (I) or pharmaceutically acceptable salt thereof twice a week. In some embodiments, the method comprises administering to the patient about 65 mg of the compound of Formula (I) or pharmaceutically acceptable salt thereof twice a week. In some embodiments, the method comprises administering to the patient about 70 mg of the compound of Formula (I) or pharmaceutically acceptable salt thereof twice a week. In some embodiments, the method comprises administering to the patient about 75 mg of the compound of Formula (I) or pharmaceutically acceptable salt thereof twice a week. In some embodiments, the method comprises administering to the patient about 80 mg of the compound of Formula (I) or pharmaceutically acceptable salt thereof twice a week. In some embodiments, the method comprises administering to the patient about 85 mg of the compound of Formula (I) or pharmaceutically acceptable salt thereof twice a week. In some embodiments, the method comprises administering to the patient about 90 mg of the compound of Formula (I) or pharmaceutically acceptable salt thereof twice a week. In some embodiments, the method comprises administering to the patient about 95 mg of the compound of Formula (I) or pharmaceutically acceptable salt thereof twice a week. In some embodiments, the method comprises administering to the patient about 100 mg of the compound of Formula (I) or pharmaceutically acceptable salt thereof twice a week. In some embodiments, the method comprises administering to the patient about 105 mg of the compound of Formula (I) or pharmaceutically acceptable salt thereof twice a week. In some embodiments, the method comprises administering to the patient about 110 mg of the compound of Formula (I) or pharmaceutically acceptable salt thereof twice a week. In some embodiments, the method comprises administering to the patient about 115 mg of the compound of Formula (I) or pharmaceutically acceptable salt thereof twice a week. In some embodiments, the method comprises administering to the patient about 120 mg of the compound of Formula (I) or pharmaceutically acceptable salt thereof twice a week. In some embodiments, the method comprises administering to the patient about 125 mg of the compound of Formula (I) or pharmaceutically acceptable salt thereof twice a week. In some embodiments, the method comprises administering to the patient about 130 mg of the compound of Formula (I) or pharmaceutically acceptable salt thereof twice a week. In some embodiments, the method comprises administering to the patient about 135 mg of the compound of Formula (I) or pharmaceutically acceptable salt thereof twice a week. In some embodiments, the method comprises administering to the patient about 140 mg of the compound of Formula (I) or pharmaceutically acceptable salt thereof twice a week. In some embodiments, the method comprises administering to the patient about 145 mg of the compound of Formula (I) or pharmaceutically acceptable salt thereof twice a week. In some embodiments, the method comprises administering to the patient about 150 mg of the compound of Formula (I) or pharmaceutically acceptable salt thereof twice a week. In some embodiments, the method comprises administering to the patient about 155 mg of the compound of Formula (I) or pharmaceutically acceptable salt thereof twice a week. In some embodiments, the method comprises administering to the patient about 160 mg of the compound of Formula (I) or pharmaceutically acceptable salt thereof twice a week. In some embodiments, the method comprises administering to the patient about 165 mg of the compound of Formula (I) or pharmaceutically acceptable salt thereof twice a week. In some embodiments, the method comprises administering to the patient about 170 mg of the compound of Formula (I) or pharmaceutically acceptable salt thereof twice a week. In some embodiments, the method comprises administering to the patient about 175 mg of the compound of Formula (I) or pharmaceutically acceptable salt thereof twice a week. In some embodiments, the method comprises administering to the patient about 180 mg of the compound of Formula (I) or pharmaceutically acceptable salt thereof twice a week. In some embodiments, the method comprises administering to the patient about 185 mg of the compound of Formula (I) or pharmaceutically acceptable salt thereof twice a week. In some embodiments, the method comprises administering to the patient about 190 mg of the compound of Formula (I) or pharmaceutically acceptable salt thereof twice a week. In some embodiments, the method comprises administering to the patient about 195 mg of the compound of Formula (I) or pharmaceutically acceptable salt thereof twice a week. In some embodiments, the method comprises administering to the patient about 200 mg of the compound of Formula (I) or pharmaceutically acceptable salt thereof twice a week.

In some embodiments, the method comprises administering to the patient about 50 mg to about 200 mg of the compound of Formula (I) or pharmaceutically acceptable salt thereof three times a week. In some embodiments, the method comprises administering to the patient about 50 mg to about 150 mg of the compound of Formula (I) or pharmaceutically acceptable salt thereof three times a week. In some embodiments, the method comprises administering to the patient about 50 mg to about 100 mg of the compound of Formula (I) or pharmaceutically acceptable salt thereof three times a week. In some embodiments, the method comprises administering to the patient about 60 mg to about 200 mg of the compound of Formula (I) or pharmaceutically acceptable salt thereof three times a week. In some embodiments, the method comprises administering to the patient about 60 mg to about 150 mg of the compound of Formula (I) or pharmaceutically acceptable salt thereof three times a week. In some embodiments, the method comprises administering to the patient about 60 mg to about 100 mg of the compound of Formula (I) or pharmaceutically acceptable salt thereof three times a week. In some embodiments, the method comprises administering to the patient about 70 mg to about 200 mg of the compound of Formula (I) or pharmaceutically acceptable salt thereof three times a week. In some embodiments, the method comprises administering to the patient about 70 mg to about 150 mg of the compound of Formula (I) or pharmaceutically acceptable salt thereof three times a week. In some embodiments, the method comprises administering to the patient about 70 mg to about 100 mg of the compound of Formula (I) or pharmaceutically acceptable salt thereof three times a week. In some embodiments, the method comprises administering to the patient about 80 mg to about 200 mg of the compound of Formula (I) or pharmaceutically acceptable salt thereof three times a week. In some embodiments, the method comprises administering to the patient about 80 mg to about 150 mg of the compound of Formula (I) or pharmaceutically acceptable salt thereof three times a week. In some embodiments, the method comprises administering to the patient about 80 mg to about 100 mg of the compound of Formula (I) or pharmaceutically acceptable salt thereof three times a week. In some embodiments, the method comprises administering to the patient about 90 mg to about 200 mg of the compound of Formula (I) or pharmaceutically acceptable salt thereof three times a week. In some embodiments, the method comprises administering to the patient about 90 mg to about 150 mg of the compound of Formula (I) or pharmaceutically acceptable salt thereof three times a week. In some embodiments, the method comprises administering to the patient about 90 mg to about 100 mg of the compound of Formula (I) or pharmaceutically acceptable salt thereof three times a week. In some embodiments, the method comprises administering to the patient about 100 mg to about 200 mg of the compound of Formula (I) or pharmaceutically acceptable salt thereof three times a week. In some embodiments, the method comprises administering to the patient about 100 mg to about 150 mg of the compound of Formula (I) or pharmaceutically acceptable salt thereof three times a week.

In some embodiments, the method comprises administering to the patient about 50 mg of the compound of Formula (I) or pharmaceutically acceptable salt thereof three times a week. In some embodiments, the method comprises administering to the patient about 55 mg of the compound of Formula (I) or pharmaceutically acceptable salt thereof three times a week. In some embodiments, the method comprises administering to the patient about 60 mg of the compound of Formula (I) or pharmaceutically acceptable salt thereof three times a week. In some embodiments, the method comprises administering to the patient about 65 mg of the compound of Formula (I) or pharmaceutically acceptable salt thereof three times a week. In some embodiments, the method comprises administering to the patient about 70 mg of the compound of Formula (I) or pharmaceutically acceptable salt thereof three times a week. In some embodiments, the method comprises administering to the patient about 75 mg of the compound of Formula (I) or pharmaceutically acceptable salt thereof three times a week. In some embodiments, the method comprises administering to the patient about 80 mg of the compound of Formula (I) or pharmaceutically acceptable salt thereof three times a week. In some embodiments, the method comprises administering to the patient about 85 mg of the compound of Formula (I) or pharmaceutically acceptable salt thereof three times a week. In some embodiments, the method comprises administering to the patient about 90 mg of the compound of Formula (I) or pharmaceutically acceptable salt thereof three times a week. In some embodiments, the method comprises administering to the patient about 95 mg of the compound of Formula (I) or pharmaceutically acceptable salt thereof three times a week. In some embodiments, the method comprises administering to the patient about 100 mg of the compound of Formula (I) or pharmaceutically acceptable salt thereof three times a week. In some embodiments, the method comprises administering to the patient about 105 mg of the compound of Formula (I) or pharmaceutically acceptable salt thereof three times a week. In some embodiments, the method comprises administering to the patient about 110 mg of the compound of Formula (I) or pharmaceutically acceptable salt thereof three times a week. In some embodiments, the method comprises administering to the patient about 115 mg of the compound of Formula (I) or pharmaceutically acceptable salt thereof three times a week. In some embodiments, the method comprises administering to the patient about 120 mg of the compound of Formula (I) or pharmaceutically acceptable salt thereof three times a week. In some embodiments, the method comprises administering to the patient about 125 mg of the compound of Formula (I) or pharmaceutically acceptable salt thereof three times a week. In some embodiments, the method comprises administering to the patient about 130 mg of the compound of Formula (I) or pharmaceutically acceptable salt thereof three times a week. In some embodiments, the method comprises administering to the patient about 135 mg of the compound of Formula (I) or pharmaceutically acceptable salt thereof three times a week. In some embodiments, the method comprises administering to the patient about 140 mg of the compound of Formula (I) or pharmaceutically acceptable salt thereof three times a week. In some embodiments, the method comprises administering to the patient about 145 mg of the compound of Formula (I) or pharmaceutically acceptable salt thereof three times a week. In some embodiments, the method comprises administering to the patient about 150 mg of the compound of Formula (I) or pharmaceutically acceptable salt thereof three times a week. In some embodiments, the method comprises administering to the patient about 155 mg of the compound of Formula (I) or pharmaceutically acceptable salt thereof three times a week. In some embodiments, the method comprises administering to the patient about 160 mg of the compound of Formula (I) or pharmaceutically acceptable salt thereof three times a week. In some embodiments, the method comprises administering to the patient about 165 mg of the compound of Formula (I) or pharmaceutically acceptable salt thereof three times a week. In some embodiments, the method comprises administering to the patient about 170 mg of the compound of Formula (I) or pharmaceutically acceptable salt thereof three times a week. In some embodiments, the method comprises administering to the patient about 175 mg of the compound of Formula (I) or pharmaceutically acceptable salt thereof three times a week. In some embodiments, the method comprises administering to the patient about 180 mg of the compound of Formula (I) or pharmaceutically acceptable salt thereof three times a week. In some embodiments, the method comprises administering to the patient about 185 mg of the compound of Formula (I) or pharmaceutically acceptable salt thereof three times a week. In some embodiments, the method comprises administering to the patient about 190 mg of the compound of Formula (I) or pharmaceutically acceptable salt thereof three times a week. In some embodiments, the method comprises administering to the patient about 195 mg of the compound of Formula (I) or pharmaceutically acceptable salt thereof three times a week. In some embodiments, the method comprises administering to the patient about 200 mg of the compound of Formula (I) or pharmaceutically acceptable salt thereof three times a week.

In some embodiments, the method comprises administering to the patient about 50 mg to about 200 mg of the compound of Formula (I) or pharmaceutically acceptable salt thereof every other day. In some embodiments, the method comprises administering to the patient about 50 mg to about 150 mg of the compound of Formula (I) or pharmaceutically acceptable salt thereof every other day. In some embodiments, the method comprises administering to the patient about 50 mg to about 100 mg of the compound of Formula (I) or pharmaceutically acceptable salt thereof every other day. In some embodiments, the method comprises administering to the patient about 60 mg to about 200 mg of the compound of Formula (I) or pharmaceutically acceptable salt thereof every other day. In some embodiments, the method comprises administering to the patient about 60 mg to about 150 mg of the compound of Formula (I) or pharmaceutically acceptable salt thereof every other day. In some embodiments, the method comprises administering to the patient about 60 mg to about 100 mg of the compound of Formula (I) or pharmaceutically acceptable salt thereof every other day. In some embodiments, the method comprises administering to the patient about 70 mg to about 200 mg of the compound of Formula (I) or pharmaceutically acceptable salt thereof every other day. In some embodiments, the method comprises administering to the patient about 70 mg to about 150 mg of the compound of Formula (I) or pharmaceutically acceptable salt thereof every other day. In some embodiments, the method comprises administering to the patient about 70 mg to about 100 mg of the compound of Formula (I) or pharmaceutically acceptable salt thereof every other day. In some embodiments, the method comprises administering to the patient about 80 mg to about 200 mg of the compound of Formula (I) or pharmaceutically acceptable salt thereof every other day. In some embodiments, the method comprises administering to the patient about 80 mg to about 150 mg of the compound of Formula (I) or pharmaceutically acceptable salt thereof every other day. In some embodiments, the method comprises administering to the patient about 80 mg to about 100 mg of the compound of Formula (I) or pharmaceutically acceptable salt thereof every other day. In some embodiments, the method comprises administering to the patient about 90 mg to about 200 mg of the compound of Formula (I) or pharmaceutically acceptable salt thereof every other day. In some embodiments, the method comprises administering to the patient about 90 mg to about 150 mg of the compound of Formula (I) or pharmaceutically acceptable salt thereof every other day. In some embodiments, the method comprises administering to the patient about 90 mg to about 100 mg of the compound of Formula (I) or pharmaceutically acceptable salt thereof every other day. In some embodiments, the method comprises administering to the patient about 100 mg to about 200 mg of the compound of Formula (I) or pharmaceutically acceptable salt thereof every other day. In some embodiments, the method comprises administering to the patient about 100 mg to about 150 mg of the compound of Formula (I) or pharmaceutically acceptable salt thereof every other day.

In some embodiments, the method comprises administering to the patient about 50 mg of the compound of Formula (I) or pharmaceutically acceptable salt thereof every other day. In some embodiments, the method comprises administering to the patient about 55 mg of the compound of Formula (I) or pharmaceutically acceptable salt thereof every other day. In some embodiments, the method comprises administering to the patient about 60 mg of the compound of Formula (I) or pharmaceutically acceptable salt thereof every other day. In some embodiments, the method comprises administering to the patient about 65 mg of the compound of Formula (I) or pharmaceutically acceptable salt thereof every other day. In some embodiments, the method comprises administering to the patient about 70 mg of the compound of Formula (I) or pharmaceutically acceptable salt thereof every other day. In some embodiments, the method comprises administering to the patient about 75 mg of the compound of Formula (I) or pharmaceutically acceptable salt thereof every other day. In some embodiments, the method comprises administering to the patient about 80 mg of the compound of Formula (I) or pharmaceutically acceptable salt thereof every other day. In some embodiments, the method comprises administering to the patient about 85 mg of the compound of Formula (I) or pharmaceutically acceptable salt thereof every other day. In some embodiments, the method comprises administering to the patient about 90 mg of the compound of Formula (I) or pharmaceutically acceptable salt thereof every other day. In some embodiments, the method comprises administering to the patient about 95 mg of the compound of Formula (I) or pharmaceutically acceptable salt thereof every other day. In some embodiments, the method comprises administering to the patient about 100 mg of the compound of Formula (I) or pharmaceutically acceptable salt thereof every other day. In some embodiments, the method comprises administering to the patient about 105 mg of the compound of Formula (I) or pharmaceutically acceptable salt thereof every other day. In some embodiments, the method comprises administering to the patient about 110 mg of the compound embodiments, the method comprises administering to the patient about 115 mg of the compound of Formula (I) or pharmaceutically acceptable salt thereof every other day. In some embodiments, the method comprises administering to the patient about 120 mg of the compound of Formula (I) or pharmaceutically acceptable salt thereof every other day. In some embodiments, the method comprises administering to the patient about 125 mg of the compound of Formula (I) or pharmaceutically acceptable salt thereof every other day. In some embodiments, the method comprises administering to the patient about 130 mg of the compound of Formula (I) or pharmaceutically acceptable salt thereof every other day. In some embodiments, the method comprises administering to the patient about 135 mg of the compound of Formula (I) or pharmaceutically acceptable salt thereof every other day. In some embodiments, the method comprises administering to the patient about 140 mg of the compound of Formula (I) or pharmaceutically acceptable salt thereof every other day. In some embodiments, the method comprises administering to the patient about 145 mg of the compound of Formula (I) or pharmaceutically acceptable salt thereof every other day. In some embodiments, the method comprises administering to the patient about 150 mg of the compound of Formula (I) or pharmaceutically acceptable salt thereof every other day. In some embodiments, the method comprises administering to the patient about 155 mg of the compound of Formula (I) or pharmaceutically acceptable salt thereof every other day. In some embodiments, the method comprises administering to the patient about 160 mg of the compound of Formula (I) or pharmaceutically acceptable salt thereof every other day. In some embodiments, the method comprises administering to the patient about 165 mg of the compound of Formula (I) or pharmaceutically acceptable salt thereof every other day. In some embodiments, the method comprises administering to the patient about 170 mg of the compound of Formula (I) or pharmaceutically acceptable salt thereof every other day. In some embodiments, the method comprises administering to the patient about 175 mg of the compound of Formula (I) or pharmaceutically acceptable salt thereof every other day. In some embodiments, the method comprises administering to the patient about 180 mg of the compound of Formula (I) or pharmaceutically acceptable salt thereof every other day. In some embodiments, the method comprises administering to the patient about 185 mg of the compound of Formula (I) or pharmaceutically acceptable salt thereof every other day. In some embodiments, the method comprises administering to the patient about 190 mg of the compound of Formula (I) or pharmaceutically acceptable salt thereof every other day. In some embodiments, the method comprises administering to the patient about 195 mg of the compound of Formula (I) or pharmaceutically acceptable salt thereof every other day. In some embodiments, the method comprises administering to the patient about 200 mg of the compound of Formula (I) or pharmaceutically acceptable salt thereof every other day.

In some embodiments, the method further comprises administering a combination agent to the patient in need thereof. In some embodiments, the combination agent is carboplatin. In some embodiments, the combination agent is paclitaxel. In some embodiments, the method further comprises administering pemetrexed to the patient in need thereof. In some embodiments, the combination agent is intravenously administered to the patient in need thereof. In some embodiments, the method further comprises administering one or more combination agents to the patient in need thereof. In some embodiments, the method further comprises administering carboplatin and paclitaxel.

In some embodiments, the patient has previously been administered at least one prior line of therapy. In some embodiments, the at least one prior line of therapy is a platinum-based therapy. In some embodiments, the patient has previously been administered carboplatin and/or pemetrexed. In some embodiments, the patient has previously been administered cisplatin and/or pemetrexed.

The one or more combination agents administered to a patient in the methods of treating lung cancer described herein can be dosed to achieve various concentrations of the combination agents in the patient's plasma, which can be quantified by the area under the curve (AUC) for each combination agent administered, e.g., intravenously administered. In some embodiments, the combination agent (e.g., carboplatin) is intravenously administered to the patient in an amount sufficient to provide an AUC of about 5 to about 7 (e.g., an AUC of about 5 or about 6).

In some embodiments, the paclitaxel is intravenously administered to the patient, e.g., by intravenous infusion. The intravenously administered paclitaxel can be administered to a patient at various dose levels. In some embodiments, the paclitaxel is administered to the patient at about 50 mg/m² to about 150 mg/m². In some embodiments, the paclitaxel is administered to the patient at about 50 mg/m² to about 100 mg/m². In some embodiments, the paclitaxel is administered to the patient at about 60 mg/m² to about 80 mg/m². In some embodiments, the paclitaxel is administered to the patient at about 60 mg/m². In some embodiments, the paclitaxel is administered to the patient at about 80 mg/m². In some embodiments, the paclitaxel is administered to the patient at about 135 mg/m². In some embodiments, the paclitaxel is administered to the patient at about 175 mg/m².

Furthermore, the paclitaxel can be administered to the patient at various frequencies. In some embodiments, the paclitaxel is administered to the patient once per week (e.g., at about 60 mg/m² once per week or about 80 mg/m² once per week). In some embodiments, the paclitaxel is administered to the patient once every three weeks (e.g., at about 135 mg/m² once every three weeks or about 175 mg/m² once every three weeks). In some embodiments, the paclitaxel is administered to the patient by intravenous infusion once per week. In some embodiments, the paclitaxel is administered to the patient by intravenous infusion once per week for three consecutive weeks. In some embodiments, the method comprises administering paclitaxel to the patient by intravenous infusion for more than one cycle, wherein each cycle comprises administering paclitaxel to the patient by intravenous infusion once per week for three consecutive weeks (e.g., once per week for three consecutive weeks at about 60 mg/m² or about 80 mg/m²). In some embodiments, the method comprises not administering paclitaxel to the patient for at least one week between each cycle.

In some embodiments, the paclitaxel is administered to the patient by infusion once every three weeks. In some embodiments, the paclitaxel is administered to the patient for about 60 minutes once per week. In some embodiments, the paclitaxel is administered to the patient for about 3 hours once per week. In some embodiments, the paclitaxel is administered to the patient for about 60 minutes once every three weeks. In some embodiments, the paclitaxel is administered to the patient for about 3 hours once every three weeks. In some embodiments, the paclitaxel is administered to the patient by intravenous infusion for about 60 minutes once per week. In some embodiments, the paclitaxel is administered to the patient by infusion for about 3 hours once per week. In some embodiments, the paclitaxel is administered to the patient by intravenous infusion for about 60 minutes once every three weeks. In some embodiments, the paclitaxel is administered to the patient by infusion for about 3 hours once every three weeks. In some embodiments, the paclitaxel is continuously administered to the patient for about 60 minutes once per week. In some embodiments, the paclitaxel is continuously administered to the patient for about 3 hours once per week. In some embodiments, the paclitaxel is continuously administered to the patient for about 60 minutes once every three weeks. In some embodiments, the paclitaxel is continuously administered to the patient for about 3 hours once every three weeks. In some embodiments, the paclitaxel is continuously administered to the patient by infusion for about 60 minutes once per week. In some embodiments, the paclitaxel is continuously administered to the patient by intravenous infusion for about 3 hours once per week. In some embodiments, the paclitaxel is continuously administered to the patient by infusion for about 60 minutes once every three weeks. In some embodiments, the paclitaxel is continuously administered to the patient by intravenous infusion for about 3 hours once every three weeks.

A method of treating lung cancer described herein can further comprise reducing each of the amount of the compound of Formula (I) or pharmaceutically acceptable salt thereof and the combination agent administered to the patient after one or more months of administering said compound of Formula (I) or pharmaceutically acceptable salt thereof and said combination agent to the patient in need thereof.

In embodiments of the methods of treating an lung cancer described herein, the disclosure also contemplates administration of the compound of Formula (I) or a pharmaceutically acceptable salt to a patient in need thereof prior to (neo-adjuvant) or after (adjuvant) surgery (e.g., surgical treatment of a solid tumor). In some embodiments, the compound of Formula (I) or a pharmaceutically acceptable salt is administered as an adjuvant. In some embodiments, the compound of Formula (I) or a pharmaceutically acceptable salt is administered as a neo-adjuvant. In some embodiments, the compound of Formula (I) or a pharmaceutically acceptable salt is administered as a neo-adjuvant and an adjuvant. In some embodiments, the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 100 years. In some embodiments, administration of the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 5 years. In some embodiments, administration of the compound of Formula (I) or a pharmaceutically acceptable salt is administered to a patient in need thereof as a neo-adjuvant for a period of 3 months to 6 months, followed by administration of the compound of Formula (I) or a pharmaceutically acceptable salt as an adjuvant for a period from 1 day to 5 years. In some embodiments, no administration of the compound of Formula (I) or a pharmaceutically acceptable salt occurs prior to surgery. In some embodiments, the compound of Formula (I) or a pharmaceutically acceptable salt is administered as an adjuvant for a period from 1 day to 100 years. In some embodiments, the compound of Formula (I) or a pharmaceutically acceptable salt is administered as an adjuvant for a period from 1 day to 5 years.

In another embodiment, provided herein is a method for achieving at least a partial response in a patient having lung cancer, comprising administering to the patient an effective amount of the compound of Formula (I)

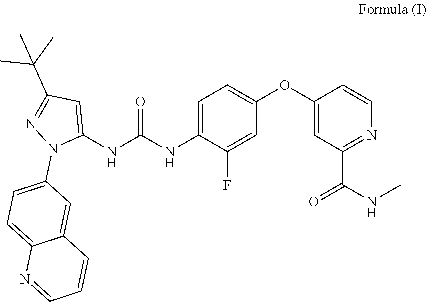 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In some embodiments, the methods further comprise administering a combination agent to the patient in need thereof. In some embodiments, the combination agent is carboplatin. In some embodiments, the combination agent is paclitaxel. In some embodiments, the method further comprises administering pemetrexed to the patient in need thereof. In some embodiments, the method further comprises administering one or more combination agents to the patient in need thereof. In some embodiments, the method further comprises administering carboplatin and paclitaxel.

In some embodiments, the patient has previously been administered at least one prior line of therapy. In some embodiments, the therapy is a platinum-based therapy. In some embodiments, the patient previously been administered carboplatin and/or pemetrexed. In some embodiments, the patient previously been administered cisplatin and/or pemetrexed.

In embodiments of the methods for achieving at least a partial response in a patient having lung cancer described herein, the compound of Formula (I) or pharmaceutically acceptable salt thereof can be orally administered to the patient in need thereof. In certain embodiments, the compound of Formula (I) or pharmaceutically acceptable salt thereof is administered to the patient daily. In some embodiments, the method comprises administering to the patient about 50 mg to about 200 mg of the compound of Formula (I) or pharmaceutically acceptable salt thereof per day. In some embodiments, the method comprises administering to the patient about 50 mg to about 150 mg of the compound of Formula (I) or pharmaceutically acceptable salt thereof per day. In some embodiments, the method comprises administering to the patient about 50 mg to about 100 mg of the compound of Formula (I) or pharmaceutically acceptable salt thereof per day. In some embodiments, the method comprises administering to the patient about 60 mg to about 200 mg of the compound of Formula (I) or pharmaceutically acceptable salt thereof per day. In some embodiments, the method comprises administering to the patient about 60 mg to about 150 mg of the compound of Formula (I) or pharmaceutically acceptable salt thereof per day. In some embodiments, the method comprises administering to the patient about 60 mg to about 100 mg of the compound of Formula (I) or pharmaceutically acceptable salt thereof per day. In some embodiments, the method comprises administering to the patient about 70 mg to about 200 mg of the compound of Formula (I) or pharmaceutically acceptable salt thereof per day. In some embodiments, the method comprises administering to the patient about 70 mg to about 150 mg of the compound of Formula (I) or pharmaceutically acceptable salt thereof per day. In some embodiments, the method comprises administering to the patient about 70 mg to about 100 mg of the compound of Formula (I) or pharmaceutically acceptable salt thereof per day. In some embodiments, the method comprises administering to the patient about 80 mg to about 200 mg of the compound of Formula (I) or pharmaceutically acceptable salt thereof per day. In some embodiments, the method comprises administering to the patient about 80 mg to about 150 mg of the compound of Formula (I) or pharmaceutically acceptable salt thereof per day. In some embodiments, the method comprises administering to the patient about 80 mg to about 100 mg of the compound of Formula (I) or pharmaceutically acceptable salt thereof per day. In some embodiments, the method comprises administering to the patient about 90 mg to about 200 mg of the compound of Formula (I) or pharmaceutically acceptable salt thereof per day. In some embodiments, the method comprises administering to the patient about 90 mg to about 150 mg of the compound of Formula (I) or pharmaceutically acceptable salt thereof per day. In some embodiments, the method comprises administering to the patient about 90 mg to about 100 mg of the compound of Formula (I) or pharmaceutically acceptable salt thereof per day. In some embodiments, the method comprises administering to the patient about 100 mg to about 200 mg of the compound of Formula (I) or pharmaceutically acceptable salt thereof per day. In some embodiments, the method comprises administering to the patient about 100 mg to about 150 mg of the compound of Formula (I) or pharmaceutically acceptable salt thereof per day.

In some embodiments, the method comprises administering to the patient about 50 mg of the compound of Formula (I) or pharmaceutically acceptable salt thereof per day. In some embodiments, the method comprises administering to the patient about 55 mg of the compound of Formula (I) or pharmaceutically acceptable salt thereof per day. In some embodiments, the method comprises administering to the patient about 60 mg of the compound of Formula (I) or pharmaceutically acceptable salt thereof per day. In some embodiments, the method comprises administering to the patient about 65 mg of the compound of Formula (I) or pharmaceutically acceptable salt thereof per day. In some embodiments, the method comprises administering to the patient about 70 mg of the compound of Formula (I) or pharmaceutically acceptable salt thereof per day. In some embodiments, the method comprises administering to the patient about 75 mg of the compound of Formula (I) or pharmaceutically acceptable salt thereof per day. In some embodiments, the method comprises administering to the patient about 80 mg of the compound of Formula (I) or pharmaceutically acceptable salt thereof per day. In some embodiments, the method comprises administering to the patient about 85 mg of the compound of Formula (I) or pharmaceutically acceptable salt thereof per day. In some embodiments, the method comprises administering to the patient about 90 mg of the compound of Formula (I) or pharmaceutically acceptable salt thereof per day. In some embodiments, the method comprises administering to the patient about 95 mg of the compound of Formula (I) or pharmaceutically acceptable salt thereof per day. In some embodiments, the method comprises administering to the patient about 100 mg of the compound of Formula (I) or pharmaceutically acceptable salt thereof per day. In some embodiments, the method comprises administering to the patient about 105 mg of the compound of Formula (I) or pharmaceutically acceptable salt thereof per day. In some embodiments, the method comprises administering to the patient about 110 mg of the compound of Formula (I) or pharmaceutically acceptable salt thereof per day. In some embodiments, the method comprises administering to the patient about 115 mg of the compound of Formula (I) or pharmaceutically acceptable salt thereof per day. In some embodiments, the method comprises administering to the patient about 120 mg of the compound of Formula (I) or pharmaceutically acceptable salt thereof per day. In some embodiments, the method comprises administering to the patient about 125 mg of the compound of Formula (I) or pharmaceutically acceptable salt thereof per day. In some embodiments, the method comprises administering to the patient about 130 mg of the compound of Formula (I) or pharmaceutically acceptable salt thereof per day. In some embodiments, the method comprises administering to the patient about 135 mg of the compound of Formula (I) or pharmaceutically acceptable salt thereof per day. In some embodiments, the method comprises administering to the patient about 140 mg of the compound of Formula (I) or pharmaceutically acceptable salt thereof per day. In some embodiments, the method comprises administering to the patient about 145 mg of the compound of Formula (I) or pharmaceutically acceptable salt thereof per day. In some embodiments, the method comprises administering to the patient about 150 mg of the compound of Formula (I) or pharmaceutically acceptable salt thereof per day. In some embodiments, the method comprises administering to the patient about 155 mg of the compound of Formula (I) or pharmaceutically acceptable salt thereof per day. In some embodiments, the method comprises administering to the patient about 160 mg of the compound of Formula (I) or pharmaceutically acceptable salt thereof per day. In some embodiments, the method comprises administering to the patient about 165 mg of the compound of Formula (I) or pharmaceutically acceptable salt thereof per day. In some embodiments, the method comprises administering to the patient about 170 mg of the compound of Formula (I) or pharmaceutically acceptable salt thereof per day. In some embodiments, the method comprises administering to the patient about 175 mg of the compound of Formula (I) or pharmaceutically acceptable salt thereof per day. In some embodiments, the method comprises administering to the patient about 180 mg of the compound of Formula (I) or pharmaceutically acceptable salt thereof per day. In some embodiments, the method comprises administering to the patient about 185 mg of the compound of Formula (I) or pharmaceutically acceptable salt thereof per day. In some embodiments, the method comprises administering to the patient about 190 mg of the compound of Formula (I) or pharmaceutically acceptable salt thereof per day. In some embodiments, the method comprises administering to the patient about 195 mg of the compound of Formula (I) or pharmaceutically acceptable salt thereof per day. In some embodiments, the method comprises administering to the patient about 200 mg of the compound of Formula (I) or pharmaceutically acceptable salt thereof per day.

In some embodiments, the method comprises administering to the patient about 50 mg to about 200 mg of the compound of Formula (I) or pharmaceutically acceptable salt thereof once per day. In some embodiments, the method comprises administering to the patient about 50 mg to about 150 mg of the compound of Formula (I) or pharmaceutically acceptable salt thereof once per day. In some embodiments, the method comprises administering to the patient about 50 mg to about 100 mg of the compound of Formula (I) or pharmaceutically acceptable salt thereof once per day. In some embodiments, the method comprises administering to the patient about 60 mg to about 200 mg of the compound of Formula (I) or pharmaceutically acceptable salt thereof once per day. In some embodiments, the method comprises administering to the patient about 60 mg to about 150 mg of the compound of Formula (I) or pharmaceutically acceptable salt thereof once per day. In some embodiments, the method comprises administering to the patient about 60 mg to about 100 mg of the compound of Formula (I) or pharmaceutically acceptable salt thereof once per day. In some embodiments, the method comprises administering to the patient about 70 mg to about 200 mg of the compound of Formula (I) or pharmaceutically acceptable salt thereof once per day. In some embodiments, the method comprises administering to the patient about 70 mg to about 150 mg of the compound of Formula (I) or pharmaceutically acceptable salt thereof once per day. In some embodiments, the method comprises administering to the patient about 70 mg to about 100 mg of the compound of Formula (I) or pharmaceutically acceptable salt thereof once per day. In some embodiments, the method comprises administering to the patient about 80 mg to about 200 mg of the compound of Formula (I) or pharmaceutically acceptable salt thereof once per day. In some embodiments, the method comprises administering to the patient about 80 mg to about 150 mg of the compound of Formula (I) or pharmaceutically acceptable salt thereof once per day. In some embodiments, the method comprises administering to the patient about 80 mg to about 100 mg of the compound of Formula (I) or pharmaceutically acceptable salt thereof once per day. In some embodiments, the method comprises administering to the patient about 90 mg to about 200 mg of the compound of Formula (I) or pharmaceutically acceptable salt thereof once per day. In some embodiments, the method comprises administering to the patient about 90 mg to about 150 mg of the compound of Formula (I) or pharmaceutically acceptable salt thereof once per day. In some embodiments, the method comprises administering to the patient about 90 mg to about 100 mg of the compound of Formula (I) or pharmaceutically acceptable salt thereof once per day. In some embodiments, the method comprises administering to the patient about 100 mg to about 200 mg of the compound of Formula (I) or pharmaceutically acceptable salt thereof once per day. In some embodiments, the method comprises administering to the patient about 100 mg to about 150 mg of the compound of Formula (I) or pharmaceutically acceptable salt thereof once per day.

In some embodiments, the method comprises administering to the patient about 50 mg of the compound of Formula (I) or pharmaceutically acceptable salt thereof once per day. In some embodiments, the method comprises administering to the patient about 55 mg of the compound of Formula (I) or pharmaceutically acceptable salt thereof once per day. In some embodiments, the method comprises administering to the patient about 60 mg of the compound of Formula (I) or pharmaceutically acceptable salt thereof once per day. In some embodiments, the method comprises administering to the patient about 65 mg of the compound of Formula (I) or pharmaceutically acceptable salt thereof once per day. In some embodiments, the method comprises administering to the patient about 70 mg of the compound of Formula (I) or pharmaceutically acceptable salt thereof once per day. In some embodiments, the method comprises administering to the patient about 75 mg of the compound of Formula (I) or pharmaceutically acceptable salt thereof once per day. In some embodiments, the method comprises administering to the patient about 80 mg of the compound of Formula (I) or pharmaceutically acceptable salt thereof once per day. In some embodiments, the method comprises administering to the patient about 85 mg of the compound of Formula (I) or pharmaceutically acceptable salt thereof once per day. In some embodiments, the method comprises administering to the patient about 90 mg of the compound of Formula (I) or pharmaceutically acceptable salt thereof once per day. In some embodiments, the method comprises administering to the patient about 95 mg of the compound of Formula (I) or pharmaceutically acceptable salt thereof once per day. In some embodiments, the method comprises administering to the patient about 100 mg of the compound of Formula (I) or pharmaceutically acceptable salt thereof once per day. In some embodiments, the method comprises administering to the patient about 105 mg of the compound of Formula (I) or pharmaceutically acceptable salt thereof once per day. In some embodiments, the method comprises administering to the patient about 110 mg of the compound of Formula (I) or pharmaceutically acceptable salt thereof once per day. In some embodiments, the method comprises administering to the patient about 115 mg of the compound of Formula (I) or pharmaceutically acceptable salt thereof once per day. In some embodiments, the method comprises administering to the patient about 120 mg of the compound of Formula (I) or pharmaceutically acceptable salt thereof once per day. In some embodiments, the method comprises administering to the patient about 125 mg of the compound of Formula (I) or pharmaceutically acceptable salt thereof once per day. In some embodiments, the method comprises administering to the patient about 130 mg of the compound of Formula (I) or pharmaceutically acceptable salt thereof once per day. In some embodiments, the method comprises administering to the patient about 135 mg of the compound of Formula (I) or pharmaceutically acceptable salt thereof once per day. In some embodiments, the method comprises administering to the patient about 140 mg of the compound of Formula (I) or pharmaceutically acceptable salt thereof once per day. In some embodiments, the method comprises administering to the patient about 145 mg of the compound of Formula (I) or pharmaceutically acceptable salt thereof once per day. In some embodiments, the method comprises administering to the patient about 150 mg of the compound of Formula (I) or pharmaceutically acceptable salt thereof once per day. In some embodiments, the method comprises administering to the patient about 155 mg of the compound of Formula (I) or pharmaceutically acceptable salt thereof once per day. In some embodiments, the method comprises administering to the patient about 160 mg of the compound of Formula (I) or pharmaceutically acceptable salt thereof once per day. In some embodiments, the method comprises administering to the patient about 165 mg of the compound of Formula (I) or pharmaceutically acceptable salt thereof once per day. In some embodiments, the method comprises administering to the patient about 170 mg of the compound of Formula (I) or pharmaceutically acceptable salt thereof once per day. In some embodiments, the method comprises administering to the patient about 175 mg of the compound of Formula (I) or pharmaceutically acceptable salt thereof once per day. In some embodiments, the method comprises administering to the patient about 180 mg of the compound of Formula (I) or pharmaceutically acceptable salt thereof once per day. In some embodiments, the method comprises administering to the patient about 185 mg of the compound of Formula (I) or pharmaceutically acceptable salt thereof once per day. In some embodiments, the method comprises administering to the patient about 190 mg of the compound of Formula (I) or pharmaceutically acceptable salt thereof once per day. In some embodiments, the method comprises administering to the patient about 195 mg of the compound of Formula (I) or pharmaceutically acceptable salt thereof once per day. In some embodiments, the method comprises administering to the patient about 200 mg of the compound of Formula (I) or pharmaceutically acceptable salt thereof once per day.

In some embodiments, the method comprises administering to the patient about 50 mg to about 100 mg of the compound of Formula (I) or pharmaceutically acceptable salt thereof twice per day. In some embodiments, the method comprises administering to the patient about 60 mg to about 100 mg of the compound of Formula (I) or pharmaceutically acceptable salt thereof twice per day. In some embodiments, the method comprises administering to the patient about 70 mg to about 100 mg of the compound of Formula (I) or pharmaceutically acceptable salt thereof twice per day. In some embodiments, the method comprises administering to the patient about 80 mg to about 100 mg of the compound of Formula (I) or pharmaceutically acceptable salt thereof twice per day. In some embodiments, the method comprises administering to the patient about 90 mg to about 100 mg of the compound of Formula (I) or pharmaceutically acceptable salt thereof twice per day.

In some embodiments, the method comprises administering to the patient about 50 mg of the compound of Formula (I) or pharmaceutically acceptable salt thereof twice per day. In some embodiments, the method comprises administering to the patient about 55 mg of the compound of Formula (I) or pharmaceutically acceptable salt thereof twice per day. In some embodiments, the method comprises administering to the patient about 60 mg of the compound of Formula (I) or pharmaceutically acceptable salt thereof twice per day. In some embodiments, the method comprises administering to the patient about 65 mg of the compound of Formula (I) or pharmaceutically acceptable salt thereof twice per day. In some embodiments, the method comprises administering to the patient about 70 mg of the compound of Formula (I) or pharmaceutically acceptable salt thereof twice per day. In some embodiments, the method comprises administering to the patient about 75 mg of the compound of Formula (I) or pharmaceutically acceptable salt thereof twice per day. In some embodiments, the method comprises administering to the patient about 80 mg of the compound of Formula (I) or pharmaceutically acceptable salt thereof twice per day. In some embodiments, the method comprises administering to the patient about 85 mg of the compound of Formula (I) or pharmaceutically acceptable salt thereof twice per day. In some embodiments, the method comprises administering to the patient about 90 mg of the compound of Formula (I) or pharmaceutically acceptable salt thereof twice per day. In some embodiments, the method comprises administering to the patient about 95 mg of the compound of Formula (I) or pharmaceutically acceptable salt thereof twice per day. In some embodiments, the method comprises administering to the patient about 100 mg of the compound of Formula (I) or pharmaceutically acceptable salt thereof twice per day.

The compound of Formula (I) or pharmaceutically acceptable salt thereof may, in another embodiment, be administered to the patient on an intermittent basis in the methods for achieving at least a partial response in a patient having lung cancer. In some embodiments, the compound of Formula (I) or pharmaceutically acceptable salt thereof is administered the patient every other day. In some embodiments, the compound of Formula (I) or pharmaceutically acceptable salt thereof is administered the patient every other day. In some embodiments, the compound of Formula (I) or pharmaceutically acceptable salt thereof is administered the patient once per week. In some embodiments, the compound of Formula (I) or pharmaceutically acceptable salt thereof is administered the patient twice per week. In some embodiments, the compound of Formula (I) or pharmaceutically acceptable salt thereof is administered the patient three times per week.

In some embodiments, the method comprises administering to the patient about 50 mg to about 200 mg of the compound of Formula (I) or pharmaceutically acceptable salt thereof once a week. In some embodiments, the method comprises administering to the patient about 50 mg to about 150 mg of the compound of Formula (I) or pharmaceutically acceptable salt thereof once a week. In some embodiments, the method comprises administering to the patient about 50 mg to about 100 mg of the compound of Formula (I) or pharmaceutically acceptable salt thereof once a week. In some embodiments, the method comprises administering to the patient about 60 mg to about 200 mg of the compound of Formula (I) or pharmaceutically acceptable salt thereof once a week. In some embodiments, the method comprises administering to the patient about 60 mg to about 150 mg of the compound of Formula (I) or pharmaceutically acceptable salt thereof once a week. In some embodiments, the method comprises administering to the patient about 60 mg to about 100 mg of the compound of Formula (I) or pharmaceutically acceptable salt thereof once a week. In some embodiments, the method comprises administering to the patient about 70 mg to about 200 mg of the compound of Formula (I) or pharmaceutically acceptable salt thereof once a week. In some embodiments, the method comprises administering to the patient about 70 mg to about 150 mg of the compound of Formula (I) or pharmaceutically acceptable salt thereof once a week. In some embodiments, the method comprises administering to the patient about 70 mg to about 100 mg of the compound of Formula (I) or pharmaceutically acceptable salt thereof once a week. In some embodiments, the method comprises administering to the patient about 80 mg to about 200 mg of the compound of Formula (I) or pharmaceutically acceptable salt thereof once a week. In some embodiments, the method comprises administering to the patient about 80 mg to about 150 mg of the compound of Formula (I) or pharmaceutically acceptable salt thereof once a week. In some embodiments, the method comprises administering to the patient about 80 mg to about 100 mg of the compound of Formula (I) or pharmaceutically acceptable salt thereof once a week. In some embodiments, the method comprises administering to the patient about 90 mg to about 200 mg of the compound of Formula (I) or pharmaceutically acceptable salt thereof once a week. In some embodiments, the method comprises administering to the patient about 90 mg to about 150 mg of the compound of Formula (I) or pharmaceutically acceptable salt thereof once a week. In some embodiments, the method comprises administering to the patient about 90 mg to about 100 mg of the compound of Formula (I) or pharmaceutically acceptable salt thereof once a week. In some embodiments, the method comprises administering to the patient about 100 mg to about 200 mg of the compound of Formula (I) or pharmaceutically acceptable salt thereof once a week. In some embodiments, the method comprises administering to the patient about 100 mg to about 150 mg of the compound of Formula (I) or pharmaceutically acceptable salt thereof once a week.

In some embodiments, the method comprises administering to the patient about 50 mg of the compound of Formula (I) or pharmaceutically acceptable salt thereof once a week. In some embodiments, the method comprises administering to the patient about 55 mg of the compound of Formula (I) or pharmaceutically acceptable salt thereof once a week. In some embodiments, the method comprises administering to the patient about 60 mg of the compound of Formula (I) or pharmaceutically acceptable salt thereof once a week. In some embodiments, the method comprises administering to the patient about 65 mg of the compound of Formula (I) or pharmaceutically acceptable salt thereof once a week. In some embodiments, the method comprises administering to the patient about 70 mg of the compound of Formula (I) or pharmaceutically acceptable salt thereof once a week. In some embodiments, the method comprises administering to the patient about 75 mg of the compound of Formula (I) or pharmaceutically acceptable salt thereof once a week. In some embodiments, the method comprises administering to the patient about 80 mg of the compound of Formula (I) or pharmaceutically acceptable salt thereof once a week. In some embodiments, the method comprises administering to the patient about 85 mg of the compound of Formula (I) or pharmaceutically acceptable salt thereof once a week. In some embodiments, the method comprises administering to the patient about 90 mg of the compound of Formula (I) or pharmaceutically acceptable salt thereof once a week. In some embodiments, the method comprises administering to the patient about 95 mg of the compound of Formula (I) or pharmaceutically acceptable salt thereof once a week. In some embodiments, the method comprises administering to the patient about 100 mg of the compound of Formula (I) or pharmaceutically acceptable salt thereof once a week. In some embodiments, the method comprises administering to the patient about 105 mg of the compound of Formula (I) or pharmaceutically acceptable salt thereof once a week. In some embodiments, the method comprises administering to the patient about 110 mg of the compound of Formula (I) or pharmaceutically acceptable salt thereof once a week. In some embodiments, the method comprises administering to the patient about 115 mg of the compound of Formula (I) or pharmaceutically acceptable salt thereof once a week. In some embodiments, the method comprises administering to the patient about 120 mg of the compound of Formula (I) or pharmaceutically acceptable salt thereof once a week. In some embodiments, the method comprises administering to the patient about 125 mg of the compound of Formula (I) or pharmaceutically acceptable salt thereof once a week. In some embodiments, the method comprises administering to the patient about 130 mg of the compound of Formula (I) or pharmaceutically acceptable salt thereof once a week. In some embodiments, the method comprises administering to the patient about 135 mg of the compound of Formula (I) or pharmaceutically acceptable salt thereof once a week. In some embodiments, the method comprises administering to the patient about 140 mg of the compound of Formula (I) or pharmaceutically acceptable salt thereof once a week. In some embodiments, the method comprises administering to the patient about 145 mg of the compound of Formula (I) or pharmaceutically acceptable salt thereof once a week. In some embodiments, the method comprises administering to the patient about 150 mg of the compound of Formula (I) or pharmaceutically acceptable salt thereof once a week. In some embodiments, the method comprises administering to the patient about 155 mg of the compound of Formula (I) or pharmaceutically acceptable salt thereof once a week. In some embodiments, the method comprises administering to the patient about 160 mg of the compound of Formula (I) or pharmaceutically acceptable salt thereof once a week. In some embodiments, the method comprises administering to the patient about 165 mg of the compound of Formula (I) or pharmaceutically acceptable salt thereof once a week. In some embodiments, the method comprises administering to the patient about 170 mg of the compound of Formula (I) or pharmaceutically acceptable salt thereof once a week. In some embodiments, the method comprises administering to the patient about 175 mg of the compound of Formula (I) or pharmaceutically acceptable salt thereof once a week. In some embodiments, the method comprises administering to the patient about 180 mg of the compound of Formula (I) or pharmaceutically acceptable salt thereof once a week. In some embodiments, the method comprises administering to the patient about 185 mg of the compound of Formula (I) or pharmaceutically acceptable salt thereof once a week. In some embodiments, the method comprises administering to the patient about 190 mg of the compound of Formula (I) or pharmaceutically acceptable salt thereof once a week. In some embodiments, the method comprises administering to the patient about 195 mg of the compound of Formula (I) or pharmaceutically acceptable salt thereof once a week. In some embodiments, the method comprises administering to the patient about 200 mg of the compound of Formula (I) or pharmaceutically acceptable salt thereof once a week.

In some embodiments, the method comprises administering to the patient about 50 mg to about 200 mg of the compound of Formula (I) or pharmaceutically acceptable salt thereof twice a week. In some embodiments, the method comprises administering to the patient about 50 mg to about 150 mg of the compound of Formula (I) or pharmaceutically acceptable salt thereof twice a week. In some embodiments, the method comprises administering to the patient about 50 mg to about 100 mg of the compound of Formula (I) or pharmaceutically acceptable salt thereof twice a week. In some embodiments, the method comprises administering to the patient about 60 mg to about 200 mg of the compound of Formula (I) or pharmaceutically acceptable salt thereof twice a week. In some embodiments, the method comprises administering to the patient about 60 mg to about 150 mg of the compound of Formula (I) or pharmaceutically acceptable salt thereof twice a week. In some embodiments, the method comprises administering to the patient about 60 mg to about 100 mg of the compound of Formula (I) or pharmaceutically acceptable salt thereof twice a week. In some embodiments, the method comprises administering to the patient about 70 mg to about 200 mg of the compound of Formula (I) or pharmaceutically acceptable salt thereof twice a week. In some embodiments, the method comprises administering to the patient about 70 mg to about 150 mg of the compound of Formula (I) or pharmaceutically acceptable salt thereof twice a week. In some embodiments, the method comprises administering to the patient about 70 mg to about 100 mg of the compound of Formula (I) or pharmaceutically acceptable salt thereof twice a week. In some embodiments, the method comprises administering to the patient about 80 mg to about 200 mg of the compound of Formula (I) or pharmaceutically acceptable salt thereof twice a week. In some embodiments, the method comprises administering to the patient about 80 mg to about 150 mg of the compound of Formula (I) or pharmaceutically acceptable salt thereof twice a week. In some embodiments, the method comprises administering to the patient about 80 mg to about 100 mg of the compound of Formula (I) or pharmaceutically acceptable salt thereof twice a week. In some embodiments, the method comprises administering to the patient about 90 mg to about 200 mg of the compound of Formula (I) or pharmaceutically acceptable salt thereof twice a week. In some embodiments, the method comprises administering to the patient about 90 mg to about 150 mg of the compound of Formula (I) or pharmaceutically acceptable salt thereof twice a week. In some embodiments, the method comprises administering to the patient about 90 mg to about 100 mg of the compound of Formula (I) or pharmaceutically acceptable salt thereof twice a week. In some embodiments, the method comprises administering to the patient about 100 mg to about 200 mg of the compound of Formula (I) or pharmaceutically acceptable salt thereof twice a week. In some embodiments, the method comprises administering to the patient about 100 mg to about 150 mg of the compound of Formula (I) or pharmaceutically acceptable salt thereof twice a week.

In some embodiments, the method comprises administering to the patient about 50 mg of the compound of Formula (I) or pharmaceutically acceptable salt thereof twice a week. In some embodiments, the method comprises administering to the patient about 55 mg of the compound of Formula (I) or pharmaceutically acceptable salt thereof twice a week. In some embodiments, the method comprises administering to the patient about 60 mg of the compound of Formula (I) or pharmaceutically acceptable salt thereof twice a week. In some embodiments, the method comprises administering to the patient about 65 mg of the compound of Formula (I) or pharmaceutically acceptable salt thereof twice a week. In some embodiments, the method comprises administering to the patient about 70 mg of the compound of Formula (I) or pharmaceutically acceptable salt thereof twice a week. In some embodiments, the method comprises administering to the patient about 75 mg of the compound of Formula (I) or pharmaceutically acceptable salt thereof twice a week. In some embodiments, the method comprises administering to the patient about 80 mg of the compound of Formula (I) or pharmaceutically acceptable salt thereof twice a week. In some embodiments, the method comprises administering to the patient about 85 mg of the compound of Formula (I) or pharmaceutically acceptable salt thereof twice a week. In some embodiments, the method comprises administering to the patient about 90 mg of the compound of Formula (I) or pharmaceutically acceptable salt thereof twice a week. In some embodiments, the method comprises administering to the patient about 95 mg of the compound of Formula (I) or pharmaceutically acceptable salt thereof twice a week. In some embodiments, the method comprises administering to the patient about 100 mg of the compound of Formula (I) or pharmaceutically acceptable salt thereof twice a week. In some embodiments, the method comprises administering to the patient about 105 mg of the compound of Formula (I) or pharmaceutically acceptable salt thereof twice a week. In some embodiments, the method comprises administering to the patient about 110 mg of the compound of Formula (I) or pharmaceutically acceptable salt thereof twice a week. In some embodiments, the method comprises administering to the patient about 115 mg of the compound of Formula (I) or pharmaceutically acceptable salt thereof twice a week. In some embodiments, the method comprises administering to the patient about 120 mg of the compound of Formula (I) or pharmaceutically acceptable salt thereof twice a week. In some embodiments, the method comprises administering to the patient about 125 mg of the compound of Formula (I) or pharmaceutically acceptable salt thereof twice a week. In some embodiments, the method comprises administering to the patient about 130 mg of the compound of Formula (I) or pharmaceutically acceptable salt thereof twice a week. In some embodiments, the method comprises administering to the patient about 135 mg of the compound of Formula (I) or pharmaceutically acceptable salt thereof twice a week. In some embodiments, the method comprises administering to the patient about 140 mg of the compound of Formula (I) or pharmaceutically acceptable salt thereof twice a week. In some embodiments, the method comprises administering to the patient about 145 mg of the compound of Formula (I) or pharmaceutically acceptable salt thereof twice a week. In some embodiments, the method comprises administering to the patient about 150 mg of the compound of Formula (I) or pharmaceutically acceptable salt thereof twice a week. In some embodiments, the method comprises administering to the patient about 155 mg of the compound of Formula (I) or pharmaceutically acceptable salt thereof twice a week. In some embodiments, the method comprises administering to the patient about 160 mg of the compound of Formula (I) or pharmaceutically acceptable salt thereof twice a week. In some embodiments, the method comprises administering to the patient about 165 mg of the compound of Formula (I) or pharmaceutically acceptable salt thereof twice a week. In some embodiments, the method comprises administering to the patient about 170 mg of the compound of Formula (I) or pharmaceutically acceptable salt thereof twice a week. In some embodiments, the method comprises administering to the patient about 175 mg of the compound of Formula (I) or pharmaceutically acceptable salt thereof twice a week. In some embodiments, the method comprises administering to the patient about 180 mg of the compound of Formula (I) or pharmaceutically acceptable salt thereof twice a week. In some embodiments, the method comprises administering to the patient about 185 mg of the compound of Formula (I) or pharmaceutically acceptable salt thereof twice a week. In some embodiments, the method comprises administering to the patient about 190 mg of the compound of Formula (I) or pharmaceutically acceptable salt thereof twice a week. In some embodiments, the method comprises administering to the patient about 195 mg of the compound of Formula (I) or pharmaceutically acceptable salt thereof twice a week. In some embodiments, the method comprises administering to the patient about 200 mg of the compound of Formula (I) or pharmaceutically acceptable salt thereof twice a week.

In some embodiments, the method comprises administering to the patient about 50 mg to about 200 mg of the compound of Formula (I) or pharmaceutically acceptable salt thereof three times a week. In some embodiments, the method comprises administering to the patient about 50 mg to about 150 mg of the compound of Formula (I) or pharmaceutically acceptable salt thereof three times a week. In some embodiments, the method comprises administering to the patient about 50 mg to about 100 mg of the compound of Formula (I) or pharmaceutically acceptable salt thereof three times a week. In some embodiments, the method comprises administering to the patient about 60 mg to about 200 mg of the compound of Formula (I) or pharmaceutically acceptable salt thereof three times a week. In some embodiments, the method comprises administering to the patient about 60 mg to about 150 mg of the compound of Formula (I) or pharmaceutically acceptable salt thereof three times a week. In some embodiments, the method comprises administering to the patient about 60 mg to about 100 mg of the compound of Formula (I) or pharmaceutically acceptable salt thereof three times a week. In some embodiments, the method comprises administering to the patient about 70 mg to about 200 mg of the compound of Formula (I) or pharmaceutically acceptable salt thereof three times a week. In some embodiments, the method comprises administering to the patient about 70 mg to about 150 mg of the compound of Formula (I) or pharmaceutically acceptable salt thereof three times a week. In some embodiments, the method comprises administering to the patient about 70 mg to about 100 mg of the compound of Formula (I) or pharmaceutically acceptable salt thereof three times a week. In some embodiments, the method comprises administering to the patient about 80 mg to about 200 mg of the compound of Formula (I) or pharmaceutically acceptable salt thereof three times a week. In some embodiments, the method comprises administering to the patient about 80 mg to about 150 mg of the compound of Formula (I) or pharmaceutically acceptable salt thereof three times a week. In some embodiments, the method comprises administering to the patient about 80 mg to about 100 mg of the compound of Formula (I) or pharmaceutically acceptable salt thereof three times a week. In some embodiments, the method comprises administering to the patient about 90 mg to about 200 mg of the compound of Formula (I) or pharmaceutically acceptable salt thereof three times a week. In some embodiments, the method comprises administering to the patient about 90 mg to about 150 mg of the compound of Formula (I) or pharmaceutically acceptable salt thereof three times a week. In some embodiments, the method comprises administering to the patient about 90 mg to about 100 mg of the compound of Formula (I) or pharmaceutically acceptable salt thereof three times a week. In some embodiments, the method comprises administering to the patient about 100 mg to about 200 mg of the compound of Formula (I) or pharmaceutically acceptable salt thereof three times a week. In some embodiments, the method comprises administering to the patient about 100 mg to about 150 mg of the compound of Formula (I) or pharmaceutically acceptable salt thereof three times a week.

In some embodiments, the method comprises administering to the patient about 50 mg of the compound of Formula (I) or pharmaceutically acceptable salt thereof three times a week. In some embodiments, the method comprises administering to the patient about 55 mg of the compound of Formula (I) or pharmaceutically acceptable salt thereof three times a week. In some embodiments, the method comprises administering to the patient about 60 mg of the compound of Formula (I) or pharmaceutically acceptable salt thereof three times a week. In some embodiments, the method comprises administering to the patient about 65 mg of the compound of Formula (I) or pharmaceutically acceptable salt thereof three times a week. In some embodiments, the method comprises administering to the patient about 70 mg of the compound of Formula (I) or pharmaceutically acceptable salt thereof three times a week. In some embodiments, the method comprises administering to the patient about 75 mg of the compound of Formula (I) or pharmaceutically acceptable salt thereof three times a week. In some embodiments, the method comprises administering to the patient about 80 mg of the compound of Formula (I) or pharmaceutically acceptable salt thereof three times a week. In some embodiments, the method comprises administering to the patient about 85 mg of the compound of Formula (I) or pharmaceutically acceptable salt thereof three times a week. In some embodiments, the method comprises administering to the patient about 90 mg of the compound of Formula (I) or pharmaceutically acceptable salt thereof three times a week. In some embodiments, the method comprises administering to the patient about 95 mg of the compound of Formula (I) or pharmaceutically acceptable salt thereof three times a week. In some embodiments, the method comprises administering to the patient about 100 mg of the compound of Formula (I) or pharmaceutically acceptable salt thereof three times a week. In some embodiments, the method comprises administering to the patient about 105 mg of the compound of Formula (I) or pharmaceutically acceptable salt thereof three times a week. In some embodiments, the method comprises administering to the patient about 110 mg of the compound of Formula (I) or pharmaceutically acceptable salt thereof three times a week. In some embodiments, the method comprises administering to the patient about 115 mg of the compound of Formula (I) or pharmaceutically acceptable salt thereof three times a week. In some embodiments, the method comprises administering to the patient about 120 mg of the compound of Formula (I) or pharmaceutically acceptable salt thereof three times a week. In some embodiments, the method comprises administering to the patient about 125 mg of the compound of Formula (I) or pharmaceutically acceptable salt thereof three times a week. In some embodiments, the method comprises administering to the patient about 130 mg of the compound of Formula (I) or pharmaceutically acceptable salt thereof three times a week. In some embodiments, the method comprises administering to the patient about 135 mg of the compound of Formula (I) or pharmaceutically acceptable salt thereof three times a week. In some embodiments, the method comprises administering to the patient about 140 mg of the compound of Formula (I) or pharmaceutically acceptable salt thereof three times a week. In some embodiments, the method comprises administering to the patient about 145 mg of the compound of Formula (I) or pharmaceutically acceptable salt thereof three times a week. In some embodiments, the method comprises administering to the patient about 150 mg of the compound of Formula (I) or pharmaceutically acceptable salt thereof three times a week. In some embodiments, the method comprises administering to the patient about 155 mg of the compound of Formula (I) or pharmaceutically acceptable salt thereof three times a week. In some embodiments, the method comprises administering to the patient about 160 mg of the compound of Formula (I) or pharmaceutically acceptable salt thereof three times a week. In some embodiments, the method comprises administering to the patient about 165 mg of the compound of Formula (I) or pharmaceutically acceptable salt thereof three times a week. In some embodiments, the method comprises administering to the patient about 170 mg of the compound of Formula (I) or pharmaceutically acceptable salt thereof three times a week. In some embodiments, the method comprises administering to the patient about 175 mg of the compound of Formula (I) or pharmaceutically acceptable salt thereof three times a week. In some embodiments, the method comprises administering to the patient about 180 mg of the compound of Formula (I) or pharmaceutically acceptable salt thereof three times a week. In some embodiments, the method comprises administering to the patient about 185 mg of the compound of Formula (I) or pharmaceutically acceptable salt thereof three times a week. In some embodiments, the method comprises administering to the patient about 190 mg of the compound of Formula (I) or pharmaceutically acceptable salt thereof three times a week. In some embodiments, the method comprises administering to the patient about 195 mg of the compound of Formula (I) or pharmaceutically acceptable salt thereof three times a week. In some embodiments, the method comprises administering to the patient about 200 mg of the compound of Formula (I) or pharmaceutically acceptable salt thereof three times a week.

In some embodiments, the method comprises administering to the patient about 50 mg to about 200 mg of the compound of Formula (I) or pharmaceutically acceptable salt thereof every other day. In some embodiments, the method comprises administering to the patient about 50 mg to about 150 mg of the compound of Formula (I) or pharmaceutically acceptable salt thereof every other day. In some embodiments, the method comprises administering to the patient about 50 mg to about 100 mg of the compound of Formula (I) or pharmaceutically acceptable salt thereof every other day. In some embodiments, the method comprises administering to the patient about 60 mg to about 200 mg of the compound of Formula (I) or pharmaceutically acceptable salt thereof every other day. In some embodiments, the method comprises administering to the patient about 60 mg to about 150 mg of the compound of Formula (I) or pharmaceutically acceptable salt thereof every other day. In some embodiments, the method comprises administering to the patient about 60 mg to about 100 mg of the compound of Formula (I) or pharmaceutically acceptable salt thereof every other day. In some embodiments, the method comprises administering to the patient about 70 mg to about 200 mg of the compound of Formula (I) or pharmaceutically acceptable salt thereof every other day. In some embodiments, the method comprises administering to the patient about 70 mg to about 150 mg of the compound of Formula (I) or pharmaceutically acceptable salt thereof every other day. In some embodiments, the method comprises administering to the patient about 70 mg to about 100 mg of the compound of Formula (I) or pharmaceutically acceptable salt thereof every other day. In some embodiments, the method comprises administering to the patient about 80 mg to about 200 mg of the compound of Formula (I) or pharmaceutically acceptable salt thereof every other day. In some embodiments, the method comprises administering to the patient about 80 mg to about 150 mg of the compound of Formula (I) or pharmaceutically acceptable salt thereof every other day. In some embodiments, the method comprises administering to the patient about 80 mg to about 100 mg of the compound of Formula (I) or pharmaceutically acceptable salt thereof every other day. In some embodiments, the method comprises administering to the patient about 90 mg to about 200 mg of the compound of Formula (I) or pharmaceutically acceptable salt thereof every other day. In some embodiments, the method comprises administering to the patient about 90 mg to about 150 mg of the compound of Formula (I) or pharmaceutically acceptable salt thereof every other day. In some embodiments, the method comprises administering to the patient about 90 mg to about 100 mg of the compound of Formula (I) or pharmaceutically acceptable salt thereof every other day. In some embodiments, the method comprises administering to the patient about 100 mg to about 200 mg of the compound of Formula (I) or pharmaceutically acceptable salt thereof every other day. In some embodiments, the method comprises administering to the patient about 100 mg to about 150 mg of the compound of Formula (I) or pharmaceutically acceptable salt thereof every other day.

In some embodiments, the method comprises administering to the patient about 50 mg of the compound of Formula (I) or pharmaceutically acceptable salt thereof every other day. In some embodiments, the method comprises administering to the patient about 55 mg of the compound of Formula (I) or pharmaceutically acceptable salt thereof every other day. In some embodiments, the method comprises administering to the patient about 60 mg of the compound of Formula (I) or pharmaceutically acceptable salt thereof every other day. In some embodiments, the method comprises administering to the patient about 65 mg of the compound of Formula (I) or pharmaceutically acceptable salt thereof every other day. In some embodiments, the method comprises administering to the patient about 70 mg of the compound of Formula (I) or pharmaceutically acceptable salt thereof every other day. In some embodiments, the method comprises administering to the patient about 75 mg of the compound of Formula (I) or pharmaceutically acceptable salt thereof every other day. In some embodiments, the method comprises administering to the patient about 80 mg of the compound of Formula (I) or pharmaceutically acceptable salt thereof every other day. In some embodiments, the method comprises administering to the patient about 85 mg of the compound of Formula (I) or pharmaceutically acceptable salt thereof every other day. In some embodiments, the method comprises administering to the patient about 90 mg of the compound of Formula (I) or pharmaceutically acceptable salt thereof every other day. In some embodiments, the method comprises administering to the patient about 95 mg of the compound embodiments, the method comprises administering to the patient about 100 mg of the compound of Formula (I) or pharmaceutically acceptable salt thereof every other day. In some embodiments, the method comprises administering to the patient about 105 mg of the compound of Formula (I) or pharmaceutically acceptable salt thereof every other day. In some embodiments, the method comprises administering to the patient about 110 mg of the compound of Formula (I) or pharmaceutically acceptable salt thereof every other day. In some embodiments, the method comprises administering to the patient about 115 mg of the compound of Formula (I) or pharmaceutically acceptable salt thereof every other day. In some embodiments, the method comprises administering to the patient about 120 mg of the compound of Formula (I) or pharmaceutically acceptable salt thereof every other day. In some embodiments, the method comprises administering to the patient about 125 mg of the compound of Formula (I) or pharmaceutically acceptable salt thereof every other day. In some embodiments, the method comprises administering to the patient about 130 mg of the compound of Formula (I) or pharmaceutically acceptable salt thereof every other day. In some embodiments, the method comprises administering to the patient about 135 mg of the compound of Formula (I) or pharmaceutically acceptable salt thereof every other day. In some embodiments, the method comprises administering to the patient about 140 mg of the compound of Formula (I) or pharmaceutically acceptable salt thereof every other day. In some embodiments, the method comprises administering to the patient about 145 mg of the compound of Formula (I) or pharmaceutically acceptable salt thereof every other day. In some embodiments, the method comprises administering to the patient about 150 mg of the compound of Formula (I) or pharmaceutically acceptable salt thereof every other day. In some embodiments, the method comprises administering to the patient about 155 mg of the compound of Formula (I) or pharmaceutically acceptable salt thereof every other day. In some embodiments, the method comprises administering to the patient about 160 mg of the compound of Formula (I) or pharmaceutically acceptable salt thereof every other day. In some embodiments, the method comprises administering to the patient about 165 mg of the compound of Formula (I) or pharmaceutically acceptable salt thereof every other day. In some embodiments, the method comprises administering to the patient about 170 mg of the compound of Formula (I) or pharmaceutically acceptable salt thereof every other day. In some embodiments, the method comprises administering to the patient about 175 mg of the compound of Formula (I) or pharmaceutically acceptable salt thereof every other day. In some embodiments, the method comprises administering to the patient about 180 mg of the compound of Formula (I) or pharmaceutically acceptable salt thereof every other day. In some embodiments, the method comprises administering to the patient about 185 mg of the compound of Formula (I) or pharmaceutically acceptable salt thereof every other day. In some embodiments, the method comprises administering to the patient about 190 mg of the compound of Formula (I) or pharmaceutically acceptable salt thereof every other day. In some embodiments, the method comprises administering to the patient about 195 mg of the compound of Formula (I) or pharmaceutically acceptable salt thereof every other day. In some embodiments, the method comprises administering to the patient about 200 mg of the compound of Formula (I) or pharmaceutically acceptable salt thereof every other day.

In some embodiments, the methods further comprise administering a combination agent to the patient in need thereof. In some embodiments, the combination agent is carboplatin. In some embodiments, the combination agent is paclitaxel. In some embodiments, the method further comprises administering pemetrexed to the patient in need thereof. In some embodiments, the method further comprises administering one or more combination agents to the patient in need thereof. In some embodiments, the method further comprises administering carboplatin and paclitaxel.

In some embodiments, the combination agent is intravenously administered to the patient in need thereof. The one or more combination agents administered to a patient in the methods of achieving at least a partial response in patients having lung cancer described herein can be dosed to achieve various concentrations of the combination agents in the patient's plasma, which can be quantified by the area under the curve (AUC) for each combination agent administered, e.g., intravenously administered. In some embodiments, the combination agent, (e.g., carboplatin) is intravenously administered to the patient in an amount sufficient to provide an AUC of about 5 to about 7 (e.g., an AUC of about 5 or about 6). In some embodiments, the method comprises reducing each of the amount of the compound of Formula (I) or pharmaceutically acceptable salt thereof and the combination agent administered to the patient after one or more months of administering to the patient in need thereof.

In some embodiments, the paclitaxel is intravenously administered to the patient, e.g., by intravenous infusion. The intravenously administered paclitaxel can be administered to a patient at various dose levels. In some embodiments, the paclitaxel is administered to the patient at about 50 mg/m² to about 150 mg/m². In some embodiments, the paclitaxel is administered to the patient at about 50 mg/m² to about 100 mg/m². In some embodiments, the paclitaxel is administered to the patient at about 60 mg/m² to about 80 mg/m². In some embodiments, the paclitaxel is administered to the patient at about 60 mg/m². In some embodiments, the paclitaxel is administered to the patient at about 80 mg/m². In some embodiments, the paclitaxel is administered to the patient at about 135 mg/m². In some embodiments, the paclitaxel is administered to the patient at about 175 mg/m².

Furthermore, the paclitaxel can be administered to the patient at various frequencies. In some embodiments, the paclitaxel is administered to the patient once per week (e.g., at about 60 mg/m² once per week or about 80 mg/m² once per week). In some embodiments, the paclitaxel is administered to the patient once every three weeks (e.g., at about 135 mg/m² once every three weeks or about 175 mg/m² once every three weeks). In some embodiments, the paclitaxel is administered to the patient by intravenous infusion once per week. In some embodiments, the paclitaxel is administered to the patient by intravenous infusion once per week for three consecutive weeks. In some embodiments, the method comprises administering paclitaxel to the patient by intravenous infusion for more than one cycle, wherein each cycle comprises administering paclitaxel to the patient by intravenous infusion once per week for three consecutive weeks (e.g., once per week for three consecutive weeks at about 60 mg/m² or about 80 mg/m²). In some embodiments, the method comprises not administering paclitaxel to the patient for at least one week between each cycle.

In some embodiments, the paclitaxel is administered to the patient by infusion once every three weeks. In some embodiments, the paclitaxel is administered to the patient for about 60 minutes once per week. In some embodiments, the paclitaxel is administered to the patient for about 3 hours once per week. In some embodiments, the paclitaxel is administered to the patient for about 60 minutes once every three weeks. In some embodiments, the paclitaxel is administered to the patient for about 3 hours once every three weeks. In some embodiments, the paclitaxel is administered to the patient by intravenous infusion for about 60 minutes once per week. In some embodiments, the paclitaxel is administered to the patient by infusion for about 3 hours once per week. In some embodiments, the paclitaxel is administered to the patient by intravenous infusion for about 60 minutes once every three weeks. In some embodiments, the paclitaxel is administered to the patient by infusion for about 3 hours once every three weeks. In some embodiments, the paclitaxel is continuously administered to the patient for about 60 minutes once per week. In some embodiments, the paclitaxel is continuously administered to the patient for about 3 hours once per week. In some embodiments, the paclitaxel is continuously administered to the patient for about 60 minutes once every three weeks. In some embodiments, the paclitaxel is continuously administered to the patient for about 3 hours once every three weeks. In some embodiments, the paclitaxel is continuously administered to the patient by infusion for about 60 minutes once per week. In some embodiments, the paclitaxel is continuously administered to the patient by intravenous infusion for about 3 hours once per week. In some embodiments, the paclitaxel is continuously administered to the patient by infusion for about 60 minutes once every three weeks. In some embodiments, the paclitaxel is continuously administered to the patient by intravenous infusion for about 3 hours once every three weeks.

The method of achieving at least a partial response in patients having lung cancer described herein described herein can further comprise reducing each of the amount of the compound of Formula (I) or pharmaceutically acceptable salt thereof and the combination agent administered to the patient after one or more months of administering said compound of Formula (I) or pharmaceutically acceptable salt thereof and said combination agent to the patient in need thereof.

In embodiments of the methods of achieving at least a partial response in a patient having an lung cancer described herein, the disclosure also contemplates administration of the compound of Formula (I) or a pharmaceutically acceptable salt to a patient in need thereof prior to (neo-adjuvant) or after (adjuvant) surgery (e.g., surgical treatment of a solid tumor). In some embodiments, the compound of Formula (I) or a pharmaceutically acceptable salt is administered as an adjuvant. In some embodiments, the compound of Formula (I) or a pharmaceutically acceptable salt is administered as a neo-adjuvant. In some embodiments, the compound of Formula (I) or a pharmaceutically acceptable salt is administered as a neo-adjuvant and an adjuvant. In some embodiments, the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 100 years. In some embodiments, administration of the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 5 years. In some embodiments, administration of the compound of Formula (I) or a pharmaceutically acceptable salt is administered to a patient in need thereof as a neo-adjuvant for a period of 3 months to 6 months, followed by administration of the compound of Formula (I) or a pharmaceutically acceptable salt as an adjuvant for a period from 1 day to 5 years. In some embodiments, no administration of the compound of Formula (I) or a pharmaceutically acceptable salt occurs prior to surgery. In some embodiments, the compound of Formula (I) or a pharmaceutically acceptable salt is administered as an adjuvant for a period from 1 day to 100 years. In some embodiments, the compound of Formula (I) or a pharmaceutically acceptable salt is administered as an adjuvant for a period from 1 day to 5 years.

In another embodiment, provided herein is a method for achieving a stable disease condition in a patient having lung cancer, comprising administering to the patient an effective amount of the compound of Formula (I)

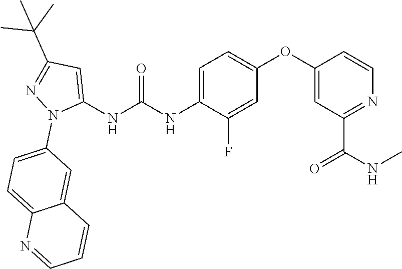 [SMILES: CNC(=O)c1cc(Oc2ccc(NC(=O)Nc3cc(C(C)(C)C)nn3-c3ccc4ncccc4c3)c(F)c2)ccn1]

or a pharmaceutically acceptable salt thereof.

In some embodiments, the method further comprises administering a combination agent to the patient in need thereof. In some embodiments, the combination agent is carboplatin. In some embodiments, the combination agent is paclitaxel. In some embodiments, the method further comprises administering pemetrexed to the patient in need thereof. In some embodiments, the method further comprises administering one or more combination agents to the patient in need thereof. In some embodiments, the method further comprises administering carboplatin and paclitaxel.

In some embodiments, the patient has previously been administered at least one prior line of therapy. In some embodiments, the therapy is a platinum-based therapy. In some embodiments, the patient previously been administered carboplatin and/or pemetrexed. In some embodiments, the patient previously been administered cisplatin and/or pemetrexed.

In embodiments of the methods for achieving a stable disease condition in a patient having lung cancer described herein, the compound of Formula (I) or pharmaceutically acceptable salt thereof can be orally administered to the patient in need thereof. In certain embodiments, the compound of Formula (I) or pharmaceutically acceptable salt thereof is administered to the patient daily. In some embodiments, the method comprises administering to the patient about 50 mg to about 200 mg of the compound of Formula (I) or pharmaceutically acceptable salt thereof per day. In some embodiments, the method comprises administering to the patient about 50 mg to about 150 mg of the compound of Formula (I) or pharmaceutically acceptable salt thereof per day. In some embodiments, the method comprises administering to the patient about 50 mg to about 100 mg of the compound of Formula (I) or pharmaceutically acceptable salt thereof per day. In some embodiments, the method comprises administering to the patient about 60 mg to about 200 mg of the compound of Formula (I) or pharmaceutically acceptable salt thereof per day. In some embodiments, the method comprises administering to the patient about 60 mg to about 150 mg of the compound of Formula (I) or pharmaceutically acceptable salt thereof per day. In some embodiments, the method comprises administering to the patient about 60 mg to about 100 mg of the compound of Formula (I) or pharmaceutically acceptable salt thereof per day. In some embodiments, the method comprises administering to the patient about 70 mg to about 200 mg of the compound of Formula (I) or pharmaceutically acceptable salt thereof per day. In some embodiments, the method comprises administering to the patient about 70 mg to about 150 mg of the compound of Formula (I) or pharmaceutically acceptable salt thereof per day. In some embodiments, the method comprises administering to the patient about 70 mg to about 100 mg of the compound of Formula (I) or pharmaceutically acceptable salt thereof per day. In some embodiments, the method comprises administering to the patient about 80 mg to about 200 mg of the compound of Formula (I) or pharmaceutically acceptable salt thereof per day. In some embodiments, the method comprises administering to the patient about 80 mg to about 150 mg of the compound of Formula (I) or pharmaceutically acceptable salt thereof per day. In some embodiments, the method comprises administering to the patient about 80 mg to about 100 mg of the compound of Formula (I) or pharmaceutically acceptable salt thereof per day. In some embodiments, the method comprises administering to the patient about 90 mg to about 200 mg of the compound of Formula (I) or pharmaceutically acceptable salt thereof per day. In some embodiments, the method comprises administering to the patient about 90 mg to about 150 mg of the compound of Formula (I) or pharmaceutically acceptable salt thereof per day. In some embodiments, the method comprises administering to the patient about 90 mg to about 100 mg of the compound of Formula (I) or pharmaceutically acceptable salt thereof per day. In some embodiments, the method comprises administering to the patient about 100 mg to about 200 mg of the compound of Formula (I) or pharmaceutically acceptable salt thereof per day. In some embodiments, the method comprises administering to the patient about 100 mg to about 150 mg of the compound of Formula (I) or pharmaceutically acceptable salt thereof per day.

In some embodiments, the method comprises administering to the patient about 50 mg of the compound of Formula (I) or pharmaceutically acceptable salt thereof per day. In some embodiments, the method comprises administering to the patient about 55 mg of the compound of Formula (I) or pharmaceutically acceptable salt thereof per day. In some embodiments, the method comprises administering to the patient about 60 mg of the compound of Formula (I) or pharmaceutically acceptable salt thereof per day. In some embodiments, the method comprises administering to the patient about 65 mg of the compound of Formula (I) or pharmaceutically acceptable salt thereof per day. In some embodiments, the method comprises administering to the patient about 70 mg of the compound of Formula (I) or pharmaceutically acceptable salt thereof per day. In some embodiments, the method comprises administering to the patient about 75 mg of the compound of Formula (I) or pharmaceutically acceptable salt thereof per day. In some embodiments, the method comprises administering to the patient about 80 mg of the compound of Formula (I) or pharmaceutically acceptable salt thereof per day. In some embodiments, the method comprises administering to the patient about 85 mg of the compound of Formula (I) or pharmaceutically acceptable salt thereof per day. In some embodiments, the method comprises administering to the patient about 90 mg of the compound of Formula (I) or pharmaceutically acceptable salt thereof per day. In some embodiments, the method comprises administering to the patient about 95 mg of the compound of Formula (I) or pharmaceutically acceptable salt thereof per day. In some embodiments, the method comprises administering to the patient about 100 mg of the compound of Formula (I) or pharmaceutically acceptable salt thereof per day. In some embodiments, the method comprises administering to the patient about 105 mg of the compound of Formula (I) or pharmaceutically acceptable salt thereof per day. In some embodiments, the method comprises administering to the patient about 110 mg of the compound of Formula (I) or pharmaceutically acceptable salt thereof per day. In some embodiments, the method comprises administering to the patient about 115 mg of the compound of Formula (I) or pharmaceutically acceptable salt thereof per day. In some embodiments, the method comprises administering to the patient about 120 mg of the compound of Formula (I) or pharmaceutically acceptable salt thereof per day. In some embodiments, the method comprises administering to the patient about 125 mg of the compound of Formula (I) or pharmaceutically acceptable salt thereof per day. In some embodiments, the method comprises administering to the patient about 130 mg of the compound of Formula (I) or pharmaceutically acceptable salt thereof per day. In some embodiments, the method comprises administering to the patient about 135 mg of the compound of Formula (I) or pharmaceutically acceptable salt thereof per day. In some embodiments, the method comprises administering to the patient about 140 mg of the compound of Formula (I) or pharmaceutically acceptable salt thereof per day. In some embodiments, the method comprises administering to the patient about 145 mg of the compound of Formula (I) or pharmaceutically acceptable salt thereof per day. In some embodiments, the method comprises administering to the patient about 150 mg of the compound of Formula (I) or pharmaceutically acceptable salt thereof per day. In some embodiments, the method comprises administering to the patient about 155 mg of the compound of Formula (I) or pharmaceutically acceptable salt thereof per day. In some embodiments, the method comprises administering to the patient about 160 mg of the compound of Formula (I) or pharmaceutically acceptable salt thereof per day. In some embodiments, the method comprises administering to the patient about 165 mg of the compound of Formula (I) or pharmaceutically acceptable salt thereof per day. In some embodiments, the method comprises administering to the patient about 170 mg of the compound of Formula (I) or pharmaceutically acceptable salt thereof per day. In some embodiments, the method comprises administering to the patient about 175 mg of the compound of Formula (I) or pharmaceutically acceptable salt thereof per day. In some embodiments, the method comprises administering to the patient about 180 mg of the compound of Formula (I) or pharmaceutically acceptable salt thereof per day. In some embodiments, the method comprises administering to the patient about 185 mg of the compound of Formula (I) or pharmaceutically acceptable salt thereof per day. In some embodiments, the method comprises administering to the patient about 190 mg of the compound of Formula (I) or pharmaceutically acceptable salt thereof per day. In some embodiments, the method comprises administering to the patient about 195 mg of the compound of Formula (I) or pharmaceutically acceptable salt thereof per day. In some embodiments, the method comprises administering to the patient about 200 mg of the compound of Formula (I) or pharmaceutically acceptable salt thereof per day.

In some embodiments, the method comprises administering to the patient about 50 mg to about 200 mg of the compound of Formula (I) or pharmaceutically acceptable salt thereof once per day. In some embodiments, the method comprises administering to the patient about 50 mg to about 150 mg of the compound of Formula (I) or pharmaceutically acceptable salt thereof once per day. In some embodiments, the method comprises administering to the patient about 50 mg to about 100 mg of the compound of Formula (I) or pharmaceutically acceptable salt thereof once per day. In some embodiments, the method comprises administering to the patient about 60 mg to about 200 mg of the compound of Formula (I) or pharmaceutically acceptable salt thereof once per day. In some embodiments, the method comprises administering to the patient about 60 mg to about 150 mg of the compound of Formula (I) or pharmaceutically acceptable salt thereof once per day. In some embodiments, the method comprises administering to the patient about 60 mg to about 100 mg of the compound of Formula (I) or pharmaceutically acceptable salt thereof once per day. In some embodiments, the method comprises administering to the patient about 70 mg to about 200 mg of the compound of Formula (I) or pharmaceutically acceptable salt thereof once per day. In some embodiments, the method comprises administering to the patient about 70 mg to about 150 mg of the compound of Formula (I) or pharmaceutically acceptable salt thereof once per day. In some embodiments, the method comprises administering to the patient about 70 mg to about 100 mg of the compound of Formula (I) or pharmaceutically acceptable salt thereof once per day. In some embodiments, the method comprises administering to the patient about 80 mg to about 200 mg of the compound of Formula (I) or pharmaceutically acceptable salt thereof once per day. In some embodiments, the method comprises administering to the patient about 80 mg to about 150 mg of the compound of Formula (I) or pharmaceutically acceptable salt thereof once per day. In some embodiments, the method comprises administering to the patient about 80 mg to about 100 mg of the compound of Formula (I) or pharmaceutically acceptable salt thereof once per day. In some embodiments, the method comprises administering to the patient about 90 mg to about 200 mg of the compound of Formula (I) or pharmaceutically acceptable salt thereof once per day. In some embodiments, the method comprises administering to the patient about 90 mg to about 150 mg of the compound of Formula (I) or pharmaceutically acceptable salt thereof once per day. In some embodiments, the method comprises administering to the patient about 90 mg to about 100 mg of the compound of Formula (I) or pharmaceutically acceptable salt thereof once per day. In some embodiments, the method comprises administering to the patient about 100 mg to about 200 mg of the compound of Formula (I) or pharmaceutically acceptable salt thereof once per day. In some embodiments, the method comprises administering to the patient about 100 mg to about 150 mg of the compound of Formula (I) or pharmaceutically acceptable salt thereof once per day.

In some embodiments, the method comprises administering to the patient about 50 mg of the compound of Formula (I) or pharmaceutically acceptable salt thereof once per day. In some embodiments, the method comprises administering to the patient about 55 mg of the compound of Formula (I) or pharmaceutically acceptable salt thereof once per day. In some embodiments, the method comprises administering to the patient about 60 mg of the compound of Formula (I) or pharmaceutically acceptable salt thereof once per day. In some embodiments, the method comprises administering to the patient about 65 mg of the compound of Formula (I) or pharmaceutically acceptable salt thereof once per day. In some embodiments, the method comprises administering to the patient about 70 mg of the compound of Formula (I) or pharmaceutically acceptable salt thereof once per day. In some embodiments, the method comprises administering to the patient about 75 mg of the compound of Formula (I) or pharmaceutically acceptable salt thereof once per day. In some embodiments, the method comprises administering to the patient about 80 mg of the compound of Formula (I) or pharmaceutically acceptable salt thereof once per day. In some embodiments, the method comprises administering to the patient about 85 mg of the compound of Formula (I) or pharmaceutically acceptable salt thereof once per day. In some embodiments, the method comprises administering to the patient about 90 mg of the compound of Formula (I) or pharmaceutically acceptable salt thereof once per day. In some embodiments, the method comprises administering to the patient about 95 mg of the compound of Formula (I) or pharmaceutically acceptable salt thereof once per day. In some embodiments, the method comprises administering to the patient about 100 mg of the compound of Formula (I) or pharmaceutically acceptable salt thereof once per day. In some embodiments, the method comprises administering to the patient about 105 mg of the compound of Formula (I) or pharmaceutically acceptable salt thereof once per day. In some embodiments, the method comprises administering to the patient about 110 mg of the compound of Formula (I) or pharmaceutically acceptable salt thereof once per day. In some embodiments, the method comprises administering to the patient about 115 mg of the compound of Formula (I) or pharmaceutically acceptable salt thereof once per day. In some embodiments, the method comprises administering to the patient about 120 mg of the compound of Formula (I) or pharmaceutically acceptable salt thereof once per day. In some embodiments, the method comprises administering to the patient about 125 mg of the compound of Formula (I) or pharmaceutically acceptable salt thereof once per day. In some embodiments, the method comprises administering to the patient about 130 mg of the compound of Formula (I) or pharmaceutically acceptable salt thereof once per day. In some embodiments, the method comprises administering to the patient about 135 mg of the compound of Formula (I) or pharmaceutically acceptable salt thereof once per day. In some embodiments, the method comprises administering to the patient about 140 mg of the compound of Formula (I) or pharmaceutically acceptable salt thereof once per day. In some embodiments, the method comprises administering to the patient about 145 mg of the compound of Formula (I) or pharmaceutically acceptable salt thereof once per day. In some embodiments, the method comprises administering to the patient about 150 mg of the compound of Formula (I) or pharmaceutically acceptable salt thereof once per day. In some embodiments, the method comprises administering to the patient about 155 mg of the compound of Formula (I) or pharmaceutically acceptable salt thereof once per day. In some embodiments, the method comprises administering to the patient about 160 mg of the compound of Formula (I) or pharmaceutically acceptable salt thereof once per day. In some embodiments, the method comprises administering to the patient about 165 mg of the compound of Formula (I) or pharmaceutically acceptable salt thereof once per day. In some embodiments, the method comprises administering to the patient about 170 mg of the compound of Formula (I) or pharmaceutically acceptable salt thereof once per day. In some embodiments, the method comprises administering to the patient about 175 mg of the compound of Formula (I) or pharmaceutically acceptable salt thereof once per day. In some embodiments, the method comprises administering to the patient about 180 mg of the compound of Formula (I) or pharmaceutically acceptable salt thereof once per day. In some embodiments, the method comprises administering to the patient about 185 mg of the compound of Formula (I) or pharmaceutically acceptable salt thereof once per day. In some embodiments, the method comprises administering to the patient about 190 mg of the compound of Formula (I) or pharmaceutically acceptable salt thereof once per day. In some embodiments, the method comprises administering to the patient about 195 mg of the compound of Formula (I) or pharmaceutically acceptable salt thereof once per day. In some embodiments, the method comprises administering to the patient about 200 mg of the compound of Formula (I) or pharmaceutically acceptable salt thereof once per day.

In some embodiments, the method comprises administering to the patient about 50 mg to about 100 mg of the compound of Formula (I) or pharmaceutically acceptable salt thereof twice per day. In some embodiments, the method comprises administering to the patient about 60 mg to about 100 mg of the compound of Formula (I) or pharmaceutically acceptable salt thereof twice per day. In some embodiments, the method comprises administering to the patient about 70 mg to about 100 mg of the compound of Formula (I) or pharmaceutically acceptable salt thereof twice per day. In some embodiments, the method comprises administering to the patient about 80 mg to about 100 mg of the compound of Formula (I) or pharmaceutically acceptable salt thereof twice per day. In some embodiments, the method comprises administering to the patient about 90 mg to about 100 mg of the compound of Formula (I) or pharmaceutically acceptable salt thereof twice per day.

In some embodiments, the method comprises administering to the patient about 50 mg of the compound of Formula (I) or pharmaceutically acceptable salt thereof twice per day. In some embodiments, the method comprises administering to the patient about 55 mg of the compound of Formula (I) or pharmaceutically acceptable salt thereof twice per day. In some embodiments, the method comprises administering to the patient about 60 mg of the compound of Formula (I) or pharmaceutically acceptable salt thereof twice per day. In some embodiments, the method comprises administering to the patient about 65 mg of the compound of Formula (I) or pharmaceutically acceptable salt thereof twice per day. In some embodiments, the method comprises administering to the patient about 70 mg of the compound of Formula (I) or pharmaceutically acceptable salt thereof twice per day. In some embodiments, the method comprises administering to the patient about 75 mg of the compound of Formula (I) or pharmaceutically acceptable salt thereof twice per day. In some embodiments, the method comprises administering to the patient about 80 mg of the compound of Formula (I) or pharmaceutically acceptable salt thereof twice per day. In some embodiments, the method comprises administering to the patient about 85 mg of the compound of Formula (I) or pharmaceutically acceptable salt thereof twice per day. In some embodiments, the method comprises administering to the patient about 90 mg of the compound of Formula (I) or pharmaceutically acceptable salt thereof twice per day. In some embodiments, the method comprises administering to the patient about 95 mg of the compound of Formula (I) or pharmaceutically acceptable salt thereof twice per day. In some embodiments, the method comprises administering to the patient about 100 mg of the compound of Formula (I) or pharmaceutically acceptable salt thereof twice per day.

The compound of Formula (I) or pharmaceutically acceptable salt thereof may, in another embodiment, be administered to the patient on an intermittent basis in the methods of achieving a stable disease condition in a patient having lung cancer described herein. In some embodiments, the compound of Formula (I) or pharmaceutically acceptable salt thereof is administered the patient every other day. In some embodiments, the compound of Formula (I) or pharmaceutically acceptable salt thereof is administered the patient every other day. In some embodiments, the compound of Formula (I) or pharmaceutically acceptable salt thereof is administered the patient once per week. In some embodiments, the compound of Formula (I) or pharmaceutically acceptable salt thereof is administered the patient twice per week. In some embodiments, the compound of Formula (I) or pharmaceutically acceptable salt thereof is administered the patient three times per week.

In some embodiments, the method comprises administering to the patient about 50 mg to about 200 mg of the compound of Formula (I) or pharmaceutically acceptable salt thereof once a week. In some embodiments, the method comprises administering to the patient about 50 mg to about 150 mg of the compound of Formula (I) or pharmaceutically acceptable salt thereof once a week. In some embodiments, the method comprises administering to the patient about 50 mg to about 100 mg of the compound of Formula (I) or pharmaceutically acceptable salt thereof once a week. In some embodiments, the method comprises administering to the patient about 60 mg to about 200 mg of the compound of Formula (I) or pharmaceutically acceptable salt thereof once a week. In some embodiments, the method comprises administering to the patient about 60 mg to about 150 mg of the compound of Formula (I) or pharmaceutically acceptable salt thereof once a week. In some embodiments, the method comprises administering to the patient about 60 mg to about 100 mg of the compound of Formula (I) or pharmaceutically acceptable salt thereof once a week. In some embodiments, the method comprises administering to the patient about 70 mg to about 200 mg of the compound of Formula (I) or pharmaceutically acceptable salt thereof once a week. In some embodiments, the method comprises administering to the patient about 70 mg to about 150 mg of the compound of Formula (I) or pharmaceutically acceptable salt thereof once a week. In some embodiments, the method comprises administering to the patient about 70 mg to about 100 mg of the compound of Formula (I) or pharmaceutically acceptable salt thereof once a week. In some embodiments, the method comprises administering to the patient about 80 mg to about 200 mg of the compound of Formula (I) or pharmaceutically acceptable salt thereof once a week. In some embodiments, the method comprises administering to the patient about 80 mg to about 150 mg of the compound of Formula (I) or pharmaceutically acceptable salt thereof once a week. In some embodiments, the method comprises administering to the patient about 80 mg to about 100 mg of the compound of Formula (I) or pharmaceutically acceptable salt thereof once a week. In some embodiments, the method comprises administering to the patient about 90 mg to about 200 mg of the compound of Formula (I) or pharmaceutically acceptable salt thereof once a week. In some embodiments, the method comprises administering to the patient about 90 mg to about 150 mg of the compound of Formula (I) or pharmaceutically acceptable salt thereof once a week. In some embodiments, the method comprises administering to the patient about 90 mg to about 100 mg of the compound of Formula (I) or pharmaceutically acceptable salt thereof once a week. In some embodiments, the method comprises administering to the patient about 100 mg to about 200 mg of the compound of Formula (I) or pharmaceutically acceptable salt thereof once a week. In some embodiments, the method comprises administering to the patient about 100 mg to about 150 mg of the compound of Formula (I) or pharmaceutically acceptable salt thereof once a week.

In some embodiments, the method comprises administering to the patient about 50 mg of the compound of Formula (I) or pharmaceutically acceptable salt thereof once a week. In some embodiments, the method comprises administering to the patient about 55 mg of the compound of Formula (I) or pharmaceutically acceptable salt thereof once a week. In some embodiments, the method comprises administering to the patient about 60 mg of the compound of Formula (I) or pharmaceutically acceptable salt thereof once a week. In some embodiments, the method comprises administering to the patient about 65 mg of the compound of Formula (I) or pharmaceutically acceptable salt thereof once a week. In some embodiments, the method comprises administering to the patient about 70 mg of the compound of Formula (I) or pharmaceutically acceptable salt thereof once a week. In some embodiments, the method comprises administering to the patient about 75 mg of the compound of Formula (I) or pharmaceutically acceptable salt thereof once a week. In some embodiments, the method comprises administering to the patient about 80 mg of the compound of Formula (I) or pharmaceutically acceptable salt thereof once a week. In some embodiments, the method comprises administering to the patient about 85 mg of the compound of Formula (I) or pharmaceutically acceptable salt thereof once a week. In some embodiments, the method comprises administering to the patient about 90 mg of the compound of Formula (I) or pharmaceutically acceptable salt thereof once a week. In some embodiments, the method comprises administering to the patient about 95 mg of the compound of Formula (I) or pharmaceutically acceptable salt thereof once a week. In some embodiments, the method comprises administering to the patient about 100 mg of the compound of Formula (I) or pharmaceutically acceptable salt thereof once a week. In some embodiments, the method comprises administering to the patient about 105 mg of the compound of Formula (I) or pharmaceutically acceptable salt thereof once a week. In some embodiments, the method comprises administering to the patient about 110 mg of the compound of Formula (I) or pharmaceutically acceptable salt thereof once a week. In some embodiments, the method comprises administering to the patient about 115 mg of the compound of Formula (I) or pharmaceutically acceptable salt thereof once a week. In some embodiments, the method comprises administering to the patient about 120 mg of the compound of Formula (I) or pharmaceutically acceptable salt thereof once a week. In some embodiments, the method comprises administering to the patient about 125 mg of the compound of Formula (I) or pharmaceutically acceptable salt thereof once a week. In some embodiments, the method comprises administering to the patient about 130 mg of the compound of Formula (I) or pharmaceutically acceptable salt thereof once a week. In some embodiments, the method comprises administering to the patient about 135 mg of the compound of Formula (I) or pharmaceutically acceptable salt thereof once a week. In some embodiments, the method comprises administering to the patient about 140 mg of the compound of Formula (I) or pharmaceutically acceptable salt thereof once a week. In some embodiments, the method comprises administering to the patient about 145 mg of the compound of Formula (I) or pharmaceutically acceptable salt thereof once a week. In some embodiments, the method comprises administering to the patient about 150 mg of the compound of Formula (I) or pharmaceutically acceptable salt thereof once a week. In some embodiments, the method comprises administering to the patient about 155 mg of the compound of Formula (I) or pharmaceutically acceptable salt thereof once a week. In some embodiments, the method comprises administering to the patient about 160 mg of the compound of Formula (I) or pharmaceutically acceptable salt thereof once a week. In some embodiments, the method comprises administering to the patient about 165 mg of the compound of Formula (I) or pharmaceutically acceptable salt thereof once a week. In some embodiments, the method comprises administering to the patient about 170 mg of the compound of Formula (I) or pharmaceutically acceptable salt thereof once a week. In some embodiments, the method comprises administering to the patient about 175 mg of the compound of Formula (I) or pharmaceutically acceptable salt thereof once a week. In some embodiments, the method comprises administering to the patient about 180 mg of the compound of Formula (I) or pharmaceutically acceptable salt thereof once a week. In some embodiments, the method comprises administering to the patient about 185 mg of the compound of Formula (I) or pharmaceutically acceptable salt thereof once a week. In some embodiments, the method comprises administering to the patient about 190 mg of the compound of Formula (I) or pharmaceutically acceptable salt thereof once a week. In some embodiments, the method comprises administering to the patient about 195 mg of the compound of Formula (I) or pharmaceutically acceptable salt thereof once a week. In some embodiments, the method comprises administering to the patient about 200 mg of the compound of Formula (I) or pharmaceutically acceptable salt thereof once a week.

In some embodiments, the method comprises administering to the patient about 50 mg to about 200 mg of the compound of Formula (I) or pharmaceutically acceptable salt thereof twice a week. In some embodiments, the method comprises administering to the patient about 50 mg to about 150 mg of the compound of Formula (I) or pharmaceutically acceptable salt thereof twice a week. In some embodiments, the method comprises administering to the patient about 50 mg to about 100 mg of the compound of Formula (I) or pharmaceutically acceptable salt thereof twice a week. In some embodiments, the method comprises administering to the patient about 60 mg to about 200 mg of the compound of Formula (I) or pharmaceutically acceptable salt thereof twice a week. In some embodiments, the method comprises administering to the patient about 60 mg to about 150 mg of the compound of Formula (I) or pharmaceutically acceptable salt thereof twice a week. In some embodiments, the method comprises administering to the patient about 60 mg to about 100 mg of the compound of Formula (I) or pharmaceutically acceptable salt thereof twice a week. In some embodiments, the method comprises administering to the patient about 70 mg to about 200 mg of the compound of Formula (I) or pharmaceutically acceptable salt thereof twice a week. In some embodiments, the method comprises administering to the patient about 70 mg to about 150 mg of the compound of Formula (I) or pharmaceutically acceptable salt thereof twice a week. In some embodiments, the method comprises administering to the patient about 70 mg to about 100 mg of the compound of Formula (I) or pharmaceutically acceptable salt thereof twice a week. In some embodiments, the method comprises administering to the patient about 80 mg to about 200 mg of the compound of Formula (I) or pharmaceutically acceptable salt thereof twice a week. In some embodiments, the method comprises administering to the patient about 80 mg to about 150 mg of the compound of Formula (I) or pharmaceutically acceptable salt thereof twice a week. In some embodiments, the method comprises administering to the patient about 80 mg to about 100 mg of the compound of Formula (I) or pharmaceutically acceptable salt thereof twice a week. In some embodiments, the method comprises administering to the patient about 90 mg to about 200 mg of the compound of Formula (I) or pharmaceutically acceptable salt thereof twice a week. In some embodiments, the method comprises administering to the patient about 90 mg to about 150 mg of the compound of Formula (I) or pharmaceutically acceptable salt thereof twice a week. In some embodiments, the method comprises administering to the patient about 90 mg to about 100 mg of the compound of Formula (I) or pharmaceutically acceptable salt thereof twice a week. In some embodiments, the method comprises administering to the patient about 100 mg to about 200 mg of the compound of Formula (I) or pharmaceutically acceptable salt thereof twice a week. In some embodiments, the method comprises administering to the patient about 100 mg to about 150 mg of the compound of Formula (I) or pharmaceutically acceptable salt thereof twice a week.

In some embodiments, the method comprises administering to the patient about 50 mg of the compound of Formula (I) or pharmaceutically acceptable salt thereof twice a week. In some embodiments, the method comprises administering to the patient about 55 mg of the compound of Formula (I) or pharmaceutically acceptable salt thereof twice a week. In some embodiments, the method comprises administering to the patient about 60 mg of the compound of Formula (I) or pharmaceutically acceptable salt thereof twice a week. In some embodiments, the method comprises administering to the patient about 65 mg of the compound of Formula (I) or pharmaceutically acceptable salt thereof twice a week. In some embodiments, the method comprises administering to the patient about 70 mg of the compound of Formula (I) or pharmaceutically acceptable salt thereof twice a week. In some embodiments, the method comprises administering to the patient about 75 mg of the compound of Formula (I) or pharmaceutically acceptable salt thereof twice a week. In some embodiments, the method comprises administering to the patient about 80 mg of the compound of Formula (I) or pharmaceutically acceptable salt thereof twice a week. In some embodiments, the method comprises administering to the patient about 85 mg of the compound of Formula (I) or pharmaceutically acceptable salt thereof twice a week. In some embodiments, the method comprises administering to the patient about 90 mg of the compound of Formula (I) or pharmaceutically acceptable salt thereof twice a week. In some embodiments, the method comprises administering to the patient about 95 mg of the compound of Formula (I) or pharmaceutically acceptable salt thereof twice a week. In some embodiments, the method comprises administering to the patient about 100 mg of the compound of Formula (I) or pharmaceutically acceptable salt thereof twice a week. In some embodiments, the method comprises administering to the patient about 105 mg of the compound of Formula (I) or pharmaceutically acceptable salt thereof twice a week. In some embodiments, the method comprises administering to the patient about 110 mg of the compound of Formula (I) or pharmaceutically acceptable salt thereof twice a week. In some embodiments, the method comprises administering to the patient about 115 mg of the compound of Formula (I) or pharmaceutically acceptable salt thereof twice a week. In some embodiments, the method comprises administering to the patient about 120 mg of the compound of Formula (I) or pharmaceutically acceptable salt thereof twice a week. In some embodiments, the method comprises administering to the patient about 125 mg of the compound of Formula (I) or pharmaceutically acceptable salt thereof twice a week. In some embodiments, the method comprises administering to the patient about 130 mg of the compound of Formula (I) or pharmaceutically acceptable salt thereof twice a week. In some embodiments, the method comprises administering to the patient about 135 mg of the compound of Formula (I) or pharmaceutically acceptable salt thereof twice a week. In some embodiments, the method comprises administering to the patient about 140 mg of the compound of Formula (I) or pharmaceutically acceptable salt thereof twice a week. In some embodiments, the method comprises administering to the patient about 145 mg of the compound of Formula (I) or pharmaceutically acceptable salt thereof twice a week. In some embodiments, the method comprises administering to the patient about 150 mg of the compound of Formula (I) or pharmaceutically acceptable salt thereof twice a week. In some embodiments, the method comprises administering to the patient about 155 mg of the compound of Formula (I) or pharmaceutically acceptable salt thereof twice a week. In some embodiments, the method comprises administering to the patient about 160 mg of the compound of Formula (I) or pharmaceutically acceptable salt thereof twice a week. In some embodiments, the method comprises administering to the patient about 165 mg of the compound of Formula (I) or pharmaceutically acceptable salt thereof twice a week. In some embodiments, the method comprises administering to the patient about 170 mg of the compound of Formula (I) or pharmaceutically acceptable salt thereof twice a week. In some embodiments, the method comprises administering to the patient about 175 mg of the compound of Formula (I) or pharmaceutically acceptable salt thereof twice a week. In some embodiments, the method comprises administering to the patient about 180 mg of the compound of Formula (I) or pharmaceutically acceptable salt thereof twice a week. In some embodiments, the method comprises administering to the patient about 185 mg of the compound of Formula (I) or pharmaceutically acceptable salt thereof twice a week. In some embodiments, the method comprises administering to the patient about 190 mg of the compound of Formula (I) or pharmaceutically acceptable salt thereof twice a week. In some embodiments, the method comprises administering to the patient about 195 mg of the compound of Formula (I) or pharmaceutically acceptable salt thereof twice a week. In some embodiments, the method comprises administering to the patient about 200 mg of the compound of Formula (I) or pharmaceutically acceptable salt thereof twice a week.

In some embodiments, the method comprises administering to the patient about 50 mg to about 200 mg of the compound of Formula (I) or pharmaceutically acceptable salt thereof three times a week. In some embodiments, the method comprises administering to the patient about 50 mg to about 150 mg of the compound of Formula (I) or pharmaceutically acceptable salt thereof three times a week. In some embodiments, the method comprises administering to the patient about 50 mg to about 100 mg of the compound of Formula (I) or pharmaceutically acceptable salt thereof three times a week. In some embodiments, the method comprises administering to the patient about 60 mg to about 200 mg of the compound of Formula (I) or pharmaceutically acceptable salt thereof three times a week. In some embodiments, the method comprises administering to the patient about 60 mg to about 150 mg of the compound of Formula (I) or pharmaceutically acceptable salt thereof three times a week. In some embodiments, the method comprises administering to the patient about 60 mg to about 100 mg of the compound of Formula (I) or pharmaceutically acceptable salt thereof three times a week. In some embodiments, the method comprises administering to the patient about 70 mg to about 200 mg of the compound of Formula (I) or pharmaceutically acceptable salt thereof three times a week. In some embodiments, the method comprises administering to the patient about 70 mg to about 150 mg of the compound of Formula (I) or pharmaceutically acceptable salt thereof three times a week. In some embodiments, the method comprises administering to the patient about 70 mg to about 100 mg of the compound of Formula (I) or pharmaceutically acceptable salt thereof three times a week. In some embodiments, the method comprises administering to the patient about 80 mg to about 200 mg of the compound of Formula (I) or pharmaceutically acceptable salt thereof three times a week. In some embodiments, the method comprises administering to the patient about 80 mg to about 150 mg of the compound of Formula (I) or pharmaceutically acceptable salt thereof three times a week. In some embodiments, the method comprises administering to the patient about 80 mg to about 100 mg of the compound of Formula (I) or pharmaceutically acceptable salt thereof three times a week. In some embodiments, the method comprises administering to the patient about 90 mg to about 200 mg of the compound of Formula (I) or pharmaceutically acceptable salt thereof three times a week. In some embodiments, the method comprises administering to the patient about 90 mg to about 150 mg of the compound of Formula (I) or pharmaceutically acceptable salt thereof three times a week. In some embodiments, the method comprises administering to the patient about 90 mg to about 100 mg of the compound of Formula (I) or pharmaceutically acceptable salt thereof three times a week. In some embodiments, the method comprises administering to the patient about 100 mg to about 200 mg of the compound of Formula (I) or pharmaceutically acceptable salt thereof three times a week. In some embodiments, the method comprises administering to the patient about 100 mg to about 150 mg of the compound of Formula (I) or pharmaceutically acceptable salt thereof three times a week.

In some embodiments, the method comprises administering to the patient about 50 mg of the compound of Formula (I) or pharmaceutically acceptable salt thereof three times a week. In some embodiments, the method comprises administering to the patient about 55 mg of the compound of Formula (I) or pharmaceutically acceptable salt thereof three times a week. In some embodiments, the method comprises administering to the patient about 60 mg of the compound of Formula (I) or pharmaceutically acceptable salt thereof three times a week. In some embodiments, the method comprises administering to the patient about 65 mg of the compound of Formula (I) or pharmaceutically acceptable salt thereof three times a week. In some embodiments, the method comprises administering to the patient about 70 mg of the compound of Formula (I) or pharmaceutically acceptable salt thereof three times a week. In some embodiments, the method comprises administering to the patient about 75 mg of the compound of Formula (I) or pharmaceutically acceptable salt thereof three times a week. In some embodiments, the method comprises administering to the patient about 80 mg of the compound of Formula (I) or pharmaceutically acceptable salt thereof three times a week. In some embodiments, the method comprises administering to the patient about 85 mg of the compound of Formula (I) or pharmaceutically acceptable salt thereof three times a week. In some embodiments, the method comprises administering to the patient about 90 mg of the compound of Formula (I) or pharmaceutically acceptable salt thereof three times a week. In some embodiments, the method comprises administering to the patient about 95 mg of the compound of Formula (I) or pharmaceutically acceptable salt thereof three times a week. In some embodiments, the method comprises administering to the patient about 100 mg of the compound of Formula (I) or pharmaceutically acceptable salt thereof three times a week. In some embodiments, the method comprises administering to the patient about 105 mg of the compound of Formula (I) or pharmaceutically acceptable salt thereof three times a week. In some embodiments, the method comprises administering to the patient about 110 mg of the compound of Formula (I) or pharmaceutically acceptable salt thereof three times a week. In some embodiments, the method comprises administering to the patient about 115 mg of the compound of Formula (I) or pharmaceutically acceptable salt thereof three times a week. In some embodiments, the method comprises administering to the patient about 120 mg of the compound of Formula (I) or pharmaceutically acceptable salt thereof three times a week. In some embodiments, the method comprises administering to the patient about 125 mg of the compound of Formula (I) or pharmaceutically acceptable salt thereof three times a week. In some embodiments, the method comprises administering to the patient about 130 mg of the compound of Formula (I) or pharmaceutically acceptable salt thereof three times a week. In some embodiments, the method comprises administering to the patient about 135 mg of the compound of Formula (I) or pharmaceutically acceptable salt thereof three times a week. In some embodiments, the method comprises administering to the patient about 140 mg of the compound of Formula (I) or pharmaceutically acceptable salt thereof three times a week. In some embodiments, the method comprises administering to the patient about 145 mg of the compound of Formula (I) or pharmaceutically acceptable salt thereof three times a week. In some embodiments, the method comprises administering to the patient about 150 mg of the compound of Formula (I) or pharmaceutically acceptable salt thereof three times a week. In some embodiments, the method comprises administering to the patient about 155 mg of the compound of Formula (I) or pharmaceutically acceptable salt thereof three times a week. In some embodiments, the method comprises administering to the patient about 160 mg of the compound of Formula (I) or pharmaceutically acceptable salt thereof three times a week. In some embodiments, the method comprises administering to the patient about 165 mg of the compound of Formula (I) or pharmaceutically acceptable salt thereof three times a week. In some embodiments, the method comprises administering to the patient about 170 mg of the compound of Formula (I) or pharmaceutically acceptable salt thereof three times a week. In some embodiments, the method comprises administering to the patient about 175 mg of the compound of Formula (I) or pharmaceutically acceptable salt thereof three times a week. In some embodiments, the method comprises administering to the patient about 180 mg of the compound of Formula (I) or pharmaceutically acceptable salt thereof three times a week. In some embodiments, the method comprises administering to the patient about 185 mg of the compound of Formula (I) or pharmaceutically acceptable salt thereof three times a week. In some embodiments, the method comprises administering to the patient about 190 mg of the compound of Formula (I) or pharmaceutically acceptable salt thereof three times a week. In some embodiments, the method comprises administering to the patient about 195 mg of the compound of Formula (I) or pharmaceutically acceptable salt thereof three times a week. In some embodiments, the method comprises administering to the patient about 200 mg of the compound of Formula (I) or pharmaceutically acceptable salt thereof three times a week.

In some embodiments, the method comprises administering to the patient about 50 mg to about 200 mg of the compound of Formula (I) or pharmaceutically acceptable salt thereof every other day. In some embodiments, the method comprises administering to the patient about 50 mg to about 150 mg of the compound of Formula (I) or pharmaceutically acceptable salt thereof every other day. In some embodiments, the method comprises administering to the patient about 50 mg to about 100 mg of the compound of Formula (I) or pharmaceutically acceptable salt thereof every other day. In some embodiments, the method comprises administering to the patient about 60 mg to about 200 mg of the compound of Formula (I) or pharmaceutically acceptable salt thereof every other day. In some embodiments, the method comprises administering to the patient about 60 mg to about 150 mg of the compound of Formula (I) or pharmaceutically acceptable salt thereof every other day. In some embodiments, the method comprises administering to the patient about 60 mg to about 100 mg of the compound of Formula (I) or pharmaceutically acceptable salt thereof every other day. In some embodiments, the method comprises administering to the patient about 70 mg to about 200 mg of the compound of Formula (I) or pharmaceutically acceptable salt thereof every other day. In some embodiments, the method comprises administering to the patient about 70 mg to about 150 mg of the compound of Formula (I) or pharmaceutically acceptable salt thereof every other day. In some embodiments, the method comprises administering to the patient about 70 mg to about 100 mg of the compound of Formula (I) or pharmaceutically acceptable salt thereof every other day. In some embodiments, the method comprises administering to the patient about 80 mg to about 200 mg of the compound of Formula (I) or pharmaceutically acceptable salt thereof every other day. In some embodiments, the method comprises administering to the patient about 80 mg to about 150 mg of the compound of Formula (I) or pharmaceutically acceptable salt thereof every other day. In some embodiments, the method comprises administering to the patient about 80 mg to about 100 mg of the compound of Formula (I) or pharmaceutically acceptable salt thereof every other day. In some embodiments, the method comprises administering to the patient about 90 mg to about 200 mg of the compound of Formula (I) or pharmaceutically acceptable salt thereof every other day. In some embodiments, the method comprises administering to the patient about 90 mg to about 150 mg of the compound of Formula (I) or pharmaceutically acceptable salt thereof every other day. In some embodiments, the method comprises administering to the patient about 90 mg to about 100 mg of the compound of Formula (I) or pharmaceutically acceptable salt thereof every other day. In some embodiments, the method comprises administering to the patient about 100 mg to about 200 mg of the compound of Formula (I) or pharmaceutically acceptable salt thereof every other day. In some embodiments, the method comprises administering to the patient about 100 mg to about 150 mg of the compound of Formula (I) or pharmaceutically acceptable salt thereof every other day.

In some embodiments, the method comprises administering to the patient about 50 mg of the compound of Formula (I) or pharmaceutically acceptable salt thereof every other day. In some embodiments, the method comprises administering to the patient about 55 mg of the compound of Formula (I) or pharmaceutically acceptable salt thereof every other day. In some embodiments, the method comprises administering to the patient about 60 mg of the compound of Formula (I) or pharmaceutically acceptable salt thereof every other day. In some embodiments, the method comprises administering to the patient about 65 mg of the compound of Formula (I) or pharmaceutically acceptable salt thereof every other day. In some embodiments, the method comprises administering to the patient about 70 mg of the compound of Formula (I) or pharmaceutically acceptable salt thereof every other day. In some embodiments, the method comprises administering to the patient about 75 mg of the compound of Formula (I) or pharmaceutically acceptable salt thereof every other day. In some embodiments, the method comprises administering to the patient about 80 mg of the compound embodiments, the method comprises administering to the patient about 85 mg of the compound of Formula (I) or pharmaceutically acceptable salt thereof every other day. In some embodiments, the method comprises administering to the patient about 90 mg of the compound of Formula (I) or pharmaceutically acceptable salt thereof every other day. In some embodiments, the method comprises administering to the patient about 95 mg of the compound of Formula (I) or pharmaceutically acceptable salt thereof every other day. In some embodiments, the method comprises administering to the patient about 100 mg of the compound of Formula (I) or pharmaceutically acceptable salt thereof every other day. In some embodiments, the method comprises administering to the patient about 105 mg of the compound of Formula (I) or pharmaceutically acceptable salt thereof every other day. In some embodiments, the method comprises administering to the patient about 110 mg of the compound of Formula (I) or pharmaceutically acceptable salt thereof every other day. In some embodiments, the method comprises administering to the patient about 115 mg of the compound of Formula (I) or pharmaceutically acceptable salt thereof every other day. In some embodiments, the method comprises administering to the patient about 120 mg of the compound of Formula (I) or pharmaceutically acceptable salt thereof every other day. In some embodiments, the method comprises administering to the patient about 125 mg of the compound of Formula (I) or pharmaceutically acceptable salt thereof every other day. In some embodiments, the method comprises administering to the patient about 130 mg of the compound of Formula (I) or pharmaceutically acceptable salt thereof every other day. In some embodiments, the method comprises administering to the patient about 135 mg of the compound of Formula (I) or pharmaceutically acceptable salt thereof every other day. In some embodiments, the method comprises administering to the patient about 140 mg of the compound of Formula (I) or pharmaceutically acceptable salt thereof every other day. In some embodiments, the method comprises administering to the patient about 145 mg of the compound of Formula (I) or pharmaceutically acceptable salt thereof every other day. In some embodiments, the method comprises administering to the patient about 150 mg of the compound of Formula (I) or pharmaceutically acceptable salt thereof every other day. In some embodiments, the method comprises administering to the patient about 155 mg of the compound of Formula (I) or pharmaceutically acceptable salt thereof every other day. In some embodiments, the method comprises administering to the patient about 160 mg of the compound of Formula (I) or pharmaceutically acceptable salt thereof every other day. In some embodiments, the method comprises administering to the patient about 165 mg of the compound of Formula (I) or pharmaceutically acceptable salt thereof every other day. In some embodiments, the method comprises administering to the patient about 170 mg of the compound of Formula (I) or pharmaceutically acceptable salt thereof every other day. In some embodiments, the method comprises administering to the patient about 175 mg of the compound of Formula (I) or pharmaceutically acceptable salt thereof every other day. In some embodiments, the method comprises administering to the patient about 180 mg of the compound of Formula (I) or pharmaceutically acceptable salt thereof every other day. In some embodiments, the method comprises administering to the patient about 185 mg of the compound of Formula (I) or pharmaceutically acceptable salt thereof every other day. In some embodiments, the method comprises administering to the patient about 190 mg of the compound of Formula (I) or pharmaceutically acceptable salt thereof every other day. In some embodiments, the method comprises administering to the patient about 195 mg of the compound of Formula (I) or pharmaceutically acceptable salt thereof every other day. In some embodiments, the method comprises administering to the patient about 200 mg of the compound of Formula (I) or pharmaceutically acceptable salt thereof every other day.

In some embodiments, the combination agent is intravenously administered to the patient in need thereof. The one or more combination agents administered to a patient in the methods of achieving a stable disease condition in patients having lung cancer described herein can be dosed to achieve various concentrations of the combination agents in the patient's plasma, which can be quantified by the area under the curve (AUC) for each combination agent administered, e.g., intravenously administered. In some embodiments, the combination agent, (e.g., carboplatin) is intravenously administered to the patient in an amount sufficient to provide an AUC of about 5 to about 7 (e.g., an AUC of about 5 or about 6). In some embodiments, the method comprises reducing each of the amount of the compound of Formula (I) or pharmaceutically acceptable salt thereof and the combination agent administered to the patient after one or more months of administering to the patient in need thereof.

In some embodiments, the paclitaxel is intravenously administered to the patient, e.g., by intravenous infusion. The intravenously administered paclitaxel can be administered to a patient at various dose levels. In some embodiments, the paclitaxel is administered to the patient at about 50 mg/m² to about 150 mg/m². In some embodiments, the paclitaxel is administered to the patient at about 50 mg/m² to about 100 mg/m². In some embodiments, the paclitaxel is administered to the patient at about 60 mg/m² to about 80 mg/m². In some embodiments, the paclitaxel is administered to the patient at about 60 mg/m². In some embodiments, the paclitaxel is administered to the patient at about 80 mg/m². In some embodiments, the paclitaxel is administered to the patient at about 135 mg/m². In some embodiments, the paclitaxel is administered to the patient at about 175 mg/m².

Furthermore, the paclitaxel can be administered to the patient at various frequencies. In some embodiments, the paclitaxel is administered to the patient once per week (e.g., at about 60 mg/m² once per week or about 80 mg/m² once per week). In some embodiments, the paclitaxel is administered to the patient once every three weeks (e.g., at about 135 mg/m² once every three weeks or about 175 mg/m² once every three weeks). In some embodiments, the paclitaxel is administered to the patient by intravenous infusion once per week. In some embodiments, the paclitaxel is administered to the patient by intravenous infusion once per week for three consecutive weeks. In some embodiments, the method comprises administering paclitaxel to the patient by intravenous infusion for more than one cycle, wherein each cycle comprises administering paclitaxel to the patient by intravenous infusion once per week for three consecutive weeks (e.g., once per week for three consecutive weeks at about 60 mg/m² or about 80 mg/m²). In some embodiments, the method comprises not administering paclitaxel to the patient for at least one week between each cycle.

In some embodiments, the paclitaxel is administered to the patient by infusion once every three weeks. In some embodiments, the paclitaxel is administered to the patient for about 60 minutes once per week. In some embodiments, the paclitaxel is administered to the patient for about 3 hours once per week. In some embodiments, the paclitaxel is administered to the patient for about 60 minutes once every three weeks. In some embodiments, the paclitaxel is administered to the patient for about 3 hours once every three weeks. In some embodiments, the paclitaxel is administered to the patient by intravenous infusion for about 60 minutes once per week. In some embodiments, the paclitaxel is administered to the patient by infusion for about 3 hours once per week. In some embodiments, the paclitaxel is administered to the patient by intravenous infusion for about 60 minutes once every three weeks. In some embodiments, the paclitaxel is administered to the patient by infusion for about 3 hours once every three weeks. In some embodiments, the paclitaxel is continuously administered to the patient for about 60 minutes once per week. In some embodiments, the paclitaxel is continuously administered to the patient for about 3 hours once per week. In some embodiments, the paclitaxel is continuously administered to the patient for about 60 minutes once every three weeks. In some embodiments, the paclitaxel is continuously administered to the patient for about 3 hours once every three weeks. In some embodiments, the paclitaxel is continuously administered to the patient by infusion for about 60 minutes once per week. In some embodiments, the paclitaxel is continuously administered to the patient by intravenous infusion for about 3 hours once per week. In some embodiments, the paclitaxel is continuously administered to the patient by infusion for about 60 minutes once every three weeks. In some embodiments, the paclitaxel is continuously administered to the patient by intravenous infusion for about 3 hours once every three weeks.

The method of achieving a stable disease condition in patients having lung cancer described herein can further comprise reducing each of the amount of the compound of Formula (I) or pharmaceutically acceptable salt thereof and the combination agent administered to the patient after one or more months of administering said compound of Formula (I) or pharmaceutically acceptable salt thereof and said combination agent to the patient in need thereof.

In embodiments of the methods of achieving at least a stable disease condition in a patient having an lung cancer described herein, the disclosure also contemplates administration of the compound of Formula (I) or a pharmaceutically acceptable salt to a patient in need thereof prior to (neo-adjuvant) or after (adjuvant) surgery (e.g., surgical treatment of a solid tumor). In some embodiments, the compound of Formula (I) or a pharmaceutically acceptable salt is administered as an adjuvant. In some embodiments, the compound of Formula (I) or a pharmaceutically acceptable salt is administered as a neo-adjuvant. In some embodiments, the compound of Formula (I) or a pharmaceutically acceptable salt is administered as a neo-adjuvant and an adjuvant. In some embodiments, the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 100 years. In some embodiments, administration of the compound of Formula (I) or a pharmaceutically acceptable salt is administered to a patient in need thereof as a neo-adjuvant for a period of 1 month to 6 months, followed by administration of the compound of Formula (I) or a pharmaceutically acceptable salt as an adjuvant for a period from 1 day to 5 years. In some embodiments, administration of the compound of Formula (I) or a pharmaceutically acceptable salt is administered to a patient in need thereof as a neo-adjuvant for a period of 3 months to 6 months, followed by administration of the compound of Formula (I) or a pharmaceutically acceptable salt as an adjuvant for a period from 1 day to 5 years. In some embodiments, no administration of the compound of Formula (I) or a pharmaceutically acceptable salt occurs prior to surgery. In some embodiments, the compound of Formula (I) or a pharmaceutically acceptable salt is administered as an adjuvant for a period from 1 day to 100 years. In some embodiments, the compound of Formula (I) or a pharmaceutically acceptable salt is administered as an adjuvant for a period from 1 day to 5 years.

### Combination Therapy

Compounds described herein, e.g., rebastinib or a pharmaceutically acceptable salt thereof, and pharmaceutical compositions thereof can be administered in combination with one or more additional therapeutic agents to treat a disorder described herein, such as a gynecologic carcinosarcoma, an endometrial adenocarcinoma, mesothelioma, ovarian cancer, pancreatic ductal adenocarcinoma, or lung cancer. In an embodiment, rebastinib or a pharmaceutically acceptable salt thereof and one additional therapeutic agent is administered. In an embodiment, rebastinib or a pharmaceutically acceptable salt thereof and two additional therapeutic agents are administered (e.g., rebastinib or a pharmaceutically acceptable salt thereof is administered to the patient in combination with paclitaxel and bevacizumab). In an embodiment, rebastinib or a pharmaceutically acceptable salt thereof and three additional therapeutic agents are administered.

Combination therapy can be achieved by administering two or more therapeutic agents, each of which is formulated and administered separately. For example, rebastinib or a pharmaceutically acceptable salt thereof and an additional therapeutic agent can be formulated and administered separately. Combination therapy can also be achieved by administering two or more therapeutic agents in a single formulation, for example a pharmaceutical composition comprising rebastinib or a pharmaceutically acceptable salt thereof as one therapeutic agent and one or more additional therapeutic agents. For example, rebastinib or a pharmaceutically acceptable salt thereof as defined herein and an additional therapeutic agent can be administered in a single formulation. Other combinations are also encompassed by combination therapy. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 1, 2, 3, 4, 5, 6, 7, 8, 9, or 10 weeks of each other. In some cases, even longer intervals are possible. While in many cases it is desirable that the two or more agents used in a combination therapy be present in within the patient's body at the same time, this need not be so.

Combination therapy can also include two or more administrations of one or more of the agents used in the combination using different sequencing of the component agents. For example, if agent X and agent Y are used in a combination, one could administer them sequentially in any combination one or more times, e.g., in the order X-Y-X, X-X-Y, Y-X-Y, Y—Y-X, X-X-Y-Y, etc. In some embodiments, the compound of Formula (I) or pharmaceutically acceptable salt thereof is administered prior to the administration of one or more therapeutic agents. In some embodiments, the compound of Formula (I) or pharmaceutically acceptable salt thereof is administered prior to the administration of paclitaxel.

In one embodiment, a compound such as rebastinib or pharmaceutically acceptable salt thereof is administered in combination with other therapeutic agents selected from the group consisting of chemotherapeutic agents, targeted therapeutics, biological agents, and radiotherapy.

The methods of the present disclosure can comprise administering to a patient the compound of Formula (I) or pharmaceutically acceptable salt in combination with paclitaxel. In some embodiments, the paclitaxel is provided as paclitaxel formulated for intravenous administration. In some embodiments, the paclitaxel administered to the patient is provided as nanoparticulate paclitaxel formulated for intraperitoneal administration (e.g., NANOTAX®). In some embodiments, the paclitaxel administered to the patient is provided as paclitaxel bound to human albumin (e.g., ABRAXANE®). In some embodiments, the paclitaxel administered to the patient is provided as paclitaxel bound to a fatty acid (e.g., paclitaxel bound to decosahexanoic acid, e.g., TAXOPREXIN®). In some embodiments, the paclitaxel administered to the patient is provided as paclitaxel conjugated to a polypeptide (e.g., paclitaxel bound to poly(L-glutamic acid), e.g., PACLICAL® poliglumex). In some embodiments, the paclitaxel administered to the patient is provided as a composition comprising paclitaxel and a surfactant-based derivative of retinoic acid (e.g., PACCAL®).

Other exemplary non-limiting embodiments of additional therapeutic agents that can be administered with rebastinib or a pharmaceutically acceptable salt thereof, alone or in combination with one or more other therapeutic agents, are provided below.

In an embodiment, a compound such as rebastinib or pharmaceutically acceptable salt thereof is administered in combination with one or more therapeutic agents including but not limited to anti-tubulin agents (e.g., paclitaxel, paclitaxel protein-bound particles for injectable suspension, eribulin, docetaxel, ixabepilone, and vincristine), vinorelbine, DNA-alkylating agents (e.g., cisplatin, carboplatin, oxaliplatin, cyclophosphamide, ifosfamide, and temozolomide), DNA intercalating agents (e.g., doxorubicin, pegylated liposomal doxorubicin, daunorubicin, idarubicin, and epirubicin), 5-fluorouracil, capecitabine, cytarabine, decitabine, 5-aza cytadine, gemcitabine and methotrexate.

In an embodiment, a compound such as rebastinib or pharmaceutically acceptable salt thereof is administered in combination with paclitaxel.

In an embodiment, a compound such as rebastinib or pharmaceutically acceptable salt thereof is administered in combination with carboplatin.

In an embodiment, a compound such as rebastinib or pharmaceutically acceptable salt thereof is administered in combination with one or more kinase inhibitors including but not limited to axitinib, erlotinib, gefitinib, lapatanib, lenvatinib, everolimus, ripretinib, temsirolimus, LY2835219, LEE011, PD 0332991, crizotinib, cabozantinib, sunitinib, pazopanib, sorafenib, regorafenib, axitinib, dasatinib, imatinib, nilotinib, vandetanib, vemurafenib, dabrafenib, trametinib, idelalisib, and quizartinib.

In another embodiment, a compound such as rebastinib or pharmaceutically acceptable salt thereof is administered in combination with one or more anti-estrogen agents including but not limited to tamoxifen, fulvestrant, anastrozole, letrozole, and exemestane.

In another embodiment, a compound such as rebastinib or pharmaceutically acceptable salt thereof is administered in combination with one or more anti-androgen agents including but not limited to abiraterone acetate, enzalutamide, nilutamide, bicalutamide, flutamide, cyproterone acetate.

In another embodiment, a compound such as rebastinib or pharmaceutically acceptable salt thereof is administered in combination with one or more steroid agents including but not limited to prednisone and dexamethazone.

In another embodiment, a compound such as rebastinib or pharmaceutically acceptable salt thereof is administered in combination with one or more topoisomerase I inhibitors including but not limited to irinotecan, camptothecin, and topotecan.

In another embodiment, a compound such as rebastinib or pharmaceutically acceptable salt thereof is administered in combination with one or more topoisomerase II inhibitors including but not limited to etoposide, etoposide phosphate, and mitoxantrone.

In another embodiment, a compound such as rebastinib or pharmaceutically acceptable salt thereof is administered in combination with one or more Histone Deacetylase (HDAC) inhibitors including but not limited to vorinostat, romidepsin, panobinostat, valproic acid, and belinostat.

In another embodiment, a compound such as rebastinib or pharmaceutically acceptable salt thereof is administered in combination with one or more DNA methylation inhibitors including but not limited to DZNep and 5-aza-2′-deoxycytidine.

In another embodiment, a compound such as rebastinib or pharmaceutically acceptable salt thereof is administered in combination with one or more proteasome inhibitors including but not limited to bortezomib and carfilzomib.

In another embodiment, a compound such as rebastinib or pharmaceutically acceptable salt thereof is administered in combination with one or more agents selected from the group consisting of thalidomide, lenalidomide and pomalidomide.

In another embodiment, a compound such as rebastinib or pharmaceutically acceptable salt thereof is administered in combination with one or more biological agents including but not limited to trastuzumab, ado-trastuzumab, pertuzumab, cetuximab, panitumumab, ipilimumab, anti-PD-1 agents including pembrolizumab, and nivolumab, anti-PD-L1 agents including atezolizumab (formerly MPDL3280A), durvalumab (formerly MEDI4736), avelumab, PDR001, anti-angiogenic agents including bevacizumab, AMG386, RG7716, and aflibercept, and antibody-drug-conjugates (ADCs) including brentuximab, vedotin, and trastuzumab emtansine.

In some embodiments, the additional therapeutic agent that may be administered according to the present disclosure include, but are not limited to, cytotoxic agents, cisplatin, doxorubicin, etoposide, irinotecan, topotecan, paclitaxel, docetaxel, the epothilones, tamoxifen, 5-fluorouracil, methotrexate, temozolomide, cyclophosphamide, lonafarib, tipifarnib, 4-((5-((4-(3-chlorophenyl)-3-oxopiperazin-1-yl)methyl)-1H-imidazol-1-yl)methyl)benzonitrile hydrochloride, (R)-1-((1H-imidazol-5-yl)methyl)-3-benzyl-4-(thiophen-2-ylsulfonyl)-2,3,4,5-tetrahydro-1H-benzo diazepine-7-carbonitrile, cetuximab, imatinib, interferon alfa-2b, pegylated interferon alfa-2b, aromatase combinations, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, leucovorin, oxaliplatin, pentostatine, vinblastine, vincristine, vindesine, bleomycin, dactinomycin, daunorubicin, epirubicin, idarubicin, mithramycin, deoxycoformycin, mitomycin-C, L-asparaginase, teniposide 17α-ethinyl estradiol, diethylstilbestrol, testosterone, prednisone, fluoxymesterone, dromostanolone propionate, testolactone, megestrol acetate, methylprednisolone, methyltestosterone, prednisolone, triamcinolone, chlorotrianisene, 17α-hydroxyprogesterone, aminoglutethimide, estramustine, medroxyprogesterone acetate, leuprolide acetate, flutamide, toremifene citrate, goserelin acetate, carboplatin, hydroxyurea, amsacrine, procarbazine, mitotane, mitoxantrone, levamisole, vinorelbine, anastrazole, letrozole, capecitabine, raloxifene, droloxafine, hexamethylmelamine, bevacizumab, trastuzumab, tositumomab, bortezomib, ibritumomab tiuxetan, arsenic trioxide, porfimer sodium, cetuximab, thioTEPA, altretamine, fulvestrant, exemestane, rituximab, alemtuzumab, dexamethasone, bicalutamide, chlorambucil, and valrubicin.

In some embodiments, the additional therapeutic agent that can be administered may include, without limitation, an AKT inhibitor, alkylating agent, all-trans retinoic acid, antiandrogen, azacitidine, BCL2 inhibitor, BCL-XL inhibitor, BCR-ABL inhibitor, BTK inhibitor, BTK/LCK/LYN inhibitor, CDK1/2/4/6/7/9 inhibitor, CDK4/6 inhibitor, CDK9 inhibitor, CBP/p300 inhibitor, EGFR inhibitor, endothelin receptor antagonist, RAF inhibitor, MEK inhibitor, ERK inhibitor, farnesyltransferase inhibitor, FLT3 inhibitor, glucocorticoid receptor agonist, HDM2 inhibitor, histone deacetylase inhibitor, IKKβ inhibitor, immunomodulatory drug (IMiD), ingenol, ITK inhibitor, JAK1/JAK2/JAK3/TYK2 inhibitor, MTOR inhibitor, PI3 kinase inhibitor, dual PI3 kinase/MTOR inhibitor, proteasome inhibitor, protein kinase C agonist, SUV39H1 inhibitor, TRAIL, VEGFR2 inhibitor, Wnt/β-catenin signaling inhibitor, decitabine, and anti-CD20 monoclonal antibody.

In some embodiments, the additional therapeutic agent is an immunomodulatory agent selected from the group consisting of CTLA4 inhibitors such as, but not limited to ipilimumab and tremelimumab; PD1 inhibitors such as, but not limited to pembrolizumab, and nivolumab; PDL1 inhibitors such as, but not limited to atezolizumab (formerly MPDL3280A), durvalumab (formerly MEDI4736), avelumab, PDR001; 4 1BB or 4 1BB ligand inhibitors such as, but not limited to urelumab and PF-05082566; OX40 ligand agonists such as, but not limited to MEDI6469; GITR agents such as, but not limited to TRX518; CD27 inhibitors such as, but not limited to varlilumab; TNFRSF25 or TL1A inhibitors; CD40 agonists such as, but not limited to CP-870893; HVEM or LIGHT or LTA or BTLA or CD160 inhibitors; LAG3 inhibitors such as, but not limited to BMS-986016; TIM3 inhibitors; Siglecs inhibitors; ICOS or ICOS ligand agonists; B7 H3 inhibitors such as, but not limited to MGA271; B7 H4 inhibitors; VISTA inhibitors; HHLA2 or TMIGD2 inhibitors; inhibitors of Butyrophilins, including BTNL2 inhibitors; CD244 or CD48 inhibitors; inhibitors of TIGIT and PVR family members; KIRs inhibitors such as, but not limited to lirilumab; inhibitors of ILTs and LIRs; NKG2D and NKG2A inhibitors such as, but not limited to IPH2201; inhibitors of MICA and MICB; CD244 inhibitors; CSF1R inhibitors such as, but not limited to emactuzumab, cabiralizumab, pexidartinib, ARRY382, BLZ945, DCC-3014; IDO inhibitors such as, but not limited to INCB024360; thalidomide, lenalidomide, TGFβ inhibitors such as, but not limited to galunisertib; adenosine or CD39 or CD73 inhibitors; CXCR4 or CXCL12 inhibitors such as, but not limited to ulocuplumab and (3S,6S,9S,12R,17R,20S,23S,26S,29S,34aS)-N—((S)-1-amino-5-guanidino-1-oxopentan-2-yl)-26,29-bis(4-aminobutyl)-17-((S)-2-((S)-2-((S)-2-(4-fluorobenzamido)-5-guanidinopentanamido)-5-guanidinopentanamido)-3-(naphthalen-2-yl)propanamido)-6-(3-guanidinopropyl)-3,20-bis(4-hydroxybenzyl)-1,4,7,10,18,21,24,27,30-nonaoxo-9,23-bis(3-ureidopropyl)triacontahydro-1H,16H-pyrrolo[2,1-p][1,2]dithia[5,8,11,14,17,20,23,26,29]nonaazacyclodotriacontine-12-carboxamide BKT140; phosphatidylserine inhibitors such as, but not limited to bavituximab; SIRPA or CD47 inhibitors such as, but not limited to CC-90002; VEGF inhibitors such as, but not limited to bevacizumab; and neuropilin inhibitors such as, but not limited to MNRP1685A.

In some embodiments, the additional therapeutic agent is a chemotherapeutic agent selected from the group consisting of chemotherapeutic agents including but not limited to anti-tubulin agents (paclitaxel, paclitaxel protein-bound particles for injectable suspension such as nab-paclitaxel, eribulin, docetaxel, ixabepilone, vincristine), vinorelbine, DNA-alkylating agents (including cisplatin, carboplatin, oxaliplatin, cyclophosphamide, ifosfamide, temozolomide), DNA intercalating agents (including doxorubicin, pegylated liposomal doxorubicin, daunorubicin, idarubicin, and epirubicin), 5-fluorouracil, capecitabine, cytarabine, decitabine, 5-aza cytadine, gemcitabine and methotrexate.

In some embodiments, the additional therapeutic agent is selected from the group consisting of paclitaxel, paclitaxel protein-bound particles for injectable suspension, eribulin, docetaxel, ixabepilone, vincristine, vinorelbine, cisplatin, carboplatin, oxaliplatin, cyclophosphamide, ifosfamide, temozolomide, doxorubicin, pegylated liposomal doxorubicin, daunorubicin, idarubicin, epirubicin, 5-fluorouracil, capecitabine, cytarabine, decitabine, 5-azacytadine, gemcitabine, methotrexate, erlotinib, gefitinib, lapatinib, everolimus, temsirolimus, LY2835219, LEE011, PD 0332991, crizotinib, cabozantinib, sunitinib, pazopanib, sorafenib, regorafenib, axitinib, dasatinib, imatinib, nilotinib, vemurafenib, dabrafenib, trametinib, idelasib, quizartinib, tamoxifen, fulvestrant, anastrozole, letrozole, exemestane, abiraterone acetate, enzalutamide, nilutamide, bicalutamide, flutamide, cyproterone acetate, prednisone, dexamethasone, irinotecan, camptothecin, topotecan, etoposide, etoposide phosphate, mitoxantrone, vorinostat, romidepsin, panobinostat, valproic acid, belinostat, DZNep 5-aza-2′-deoxycytidine, bortezomib, carfilzomib, thalidomide, lenalidomide, pomalidomide, trastuzumab, pertuzumab, cetuximab, panitumumab, ipilimumab, labrolizumab, nivolumab, MPDL3280A, bevacizumab, aflibercept, brentuximab vedotin, ado-trastuzumab emtansine, radiotherapy, and sipuleucel T.

In some embodiments, the additional therapeutic agent is a kinase inhibitor selected from the group consisting of erlotinib, gefitinib, lapatanib, everolimus, temsirolimus, LY2835219, LEE011, PD 0332991, crizotinib, cabozantinib, lenvatinib, sunitinib, pazopanib, sorafenib, regorafenib, axitinib, dasatinib, imatinib, nilotinib, vemurafenib, dabrafenib, trametinib, binimetinib, cobimetinib, idelalisib, and quizartinib.

In some embodiments, the additional therapeutic agent is an anti-PD1 therapeutic. Examples of anti-PD1 therapeutics that may be administered in combination with the compound of Formula (I) or pharmaceutically acceptable salt thereof or a composition comprising the compound of Formula (I) or pharmaceutically acceptable salt thereof described herein include, but are not limited to, nivolumab, pidilizumab, cemiplimab, tislelizumab, AMP-224, AMP-514, and pembrolizumab.

In some embodiments, the additional therapeutic agent is selected from the group consisting of immunomodulatory agents including but not limited to anti-PD-L1 therapeutics including atezolizumab, durvalumab, BMS-936559, and avelumab, anti-TIM3 therapeutics including TSR-022 and MBG453, anti-LAG3 therapeutics including relatlimab, LAG525, and TSR-033, CD40 agonist therapeutics including SGN-40, CP-870,893 and R07009789, anti-CD47 therapeutics including Hu5F9-G4, anti-CD20 therapeutics, anti-CD38 therapeutics, and other immunomodulatory therapeutics including thalidomide, lenalidomide, pomalidomide, prednisone, and dexamethasone. In some embodiments, the additional therapeutic agent is avelumab.

In some embodiments, the additional therapeutic agent is a chemotherapeutic agent selected from the group consisting of anti-tubulin agents (e.g., paclitaxel, paclitaxel protein-bound particles for injectable suspension, eribulin, abraxane, docetaxel, ixabepilone, taxiterem, vincristine or vinorelbine), LHRH antagonists including but not limited to leuprolide, goserelin, triptorelin, or histrelin, anti-androgen agents including but not limited to abiraterone, flutamide, bicalutamide, nilutamide, cyproterone acetate, enzalutamide, and apalutamide, anti-estrogen agents including but not limited to tamoxifen, fulvestrant, anastrozole, letrozole, and exemestane, DNA-alkylating agents (including cisplatin, carboplatin, oxaliplatin, cyclophosphamide, ifosfamide, and temozolomide), DNA intercalating agents (including doxorubicin, pegylated liposomal doxorubicin, daunorubicin, idarubicin, and epirubicin), 5-fluorouracil, capecitabine, cytarabine, decitabine, 5-aza cytadine, gemcitabine methotrexate, bortezomib, and carfilzomib.

In some embodiments, the additional therapeutic agent is selected from the group consisting of targeted therapeutics including kinase inhibitors erlotinib, gefitinib, lapatanib, everolimus, temsirolimus, abemaciclib, LEE011, palbociclib, crizotinib, cabozantinib, lenvatinib, sunitinib, pazopanib, sorafenib, regorafenib, axitinib, dasatinib, imatinib, nilotinib, vemurafenib, dabrafenib, trametinib, cobimetinib, binimetinib, idelalisib, quizartinib, avapritinib, BLU-667, BLU-263, Loxo 292, larotrectinib, and quizartinib, anti-estrogen agents including but not limited to tamoxifen, fulvestrant, anastrozole, letrozole, and exemestane, anti-androgen agents including but not limited to abiraterone acetate, enzalutamide, nilutamide, bicalutamide, flutamide, cyproterone acetate, steroid agents including but not limited to prednisone and dexamethasone, PARP inhibitors including but not limited to neraparib, olaparib, and rucaparib, topoisomerase I inhibitors including but not limited to irinotecan, camptothecin, and topotecan, topoisomerase II inhibitors including but not limited to etoposide, etoposide phosphate, and mitoxantrone, Histone Deacetylase (HDAC) inhibitors including but not limited to vorinostat, romidepsin, panobinostat, valproic acid, and belinostat, DNA methylation inhibitors including but not limited to DZNep and 5-aza-2′-deoxycytidine, proteasome inhibitors including but not limited to bortezomib and carfilzomib, thalidomide, lenalidomide, pomalidomide, biological agents including but not limited to trastuzumab, ado-trastuzumab, pertuzumab, cetuximab, panitumumab, ipilimumab, tremelimumab, vaccines including but not limited to sipuleucel-T, and radiotherapy.

In some embodiments, the additional therapeutic agent is selected from the group consisting of anti-angiogenic agents including AMG386, bevacizumab, RG7716 and aflibercept, and antibody-drug-conjugates (ADCs) including brentuximab vedotin, trastuzumab emtansine, and ADCs containing a payload such as a derivative of camptothecin, a pyrrolobenzodiazepine dimer (PBD), an indolinobenzodiazepine dimer (IGN), DM1, DM4, MMAE, or MMAF.

In some embodiments, the additional therapeutic agent is selected from a luteinizing hormone-releasing hormone (LHRH) analog, including goserelin and leuprolide.

In some embodiments, the additional therapeutic agent is selected from the group consisting of selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, lenvatinib, vandetanib, ARQ-197, MK-0457, MLN8054, PHA-739358, R-763, AT-9263, pemetrexed, erlotinib, dasatanib, nilotinib, decatanib, panitumumab, amrubicin, oregovomab, Lep-etu, nolatrexed, azd2171, batabulin, of atumtunab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)-ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258); 3-[5-(methylsulfonylpiperadinemethyl)-indolylj-quinolone, vatalanib, AG-013736, AVE-0005, the acetate salt of [D-Ser(Bu t) 6, Azgly 10] (pyro-Glu-His-Trp-Ser-Tyr-D-Ser(Bu 0-Leu-Arg-Pro-Azgly-NH₂ acetate [C₅₉H₈₄N₁₈Oi₄-(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutanide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, ipilumumab, vemurafenib, and mixtures thereof.

In some embodiments, the additional therapeutic agent is an HSP90 inhibitor (e.g., AT13387). In some embodiments, the additional therapeutic agent is cyclophosphamide. In some embodiments, the additional therapeutic agent is an AKT inhibitor (e.g., perifosine). In some embodiments, the additional therapeutic agent is a BCR-ABL inhibitor (e.g., nilotinib). In some embodiments, the additional therapeutic agent is an mTOR inhibitor (e.g., RAD001). In some embodiments, the additional therapeutic agent is an FGFR inhibitor (e.g., erdafitinib, KO947, or BGJ398). In some embodiments, the additional therapeutic agent is an anti-PDL1 therapeutic. In some embodiments, the additional therapeutic agent is a Bcl2 inhibitor (e.g., venetoclax). In some embodiments, the additional therapeutic agent is an autophagy inhibitor (e.g., hydroxychloroquine). In some embodiments, the additional therapeutic agent is a MET inhibitor.

### EXAMPLES

The following examples are set forth to illustrate the compounds, pharmaceutical compositions and methods described herein and are not to be construed in any way as limiting their scope.

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

The following abbreviations are used in the Examples provided below: BID: twice daily; RP2D: recommended phase 2 dose; MTD: maximum tolerable dose; PR: partial response; CR: complete response; ORR: objective response rate; CBR: clinical benefit rate; conc.: concentrated; DIEA: N,N-diisopropylethylamine; DMA: N,N-dimethylacetamide; DMF: N,N-dimethylformamide; DMAP: is 4-(dimethylamino)pyridine; DMSO: dimethylsulfoxide; Et₂O: diethyl ether; EtOAc: ethyl acetate; EtOH: ethanol; ESI: electrospray ionization; MS: mass spectrometry; NMR: is nuclear magnetic resonance; pet ether: petroleum ether; RT: room temperature which is also known as “ambient temp,” which will be understood to consist of a range of normal laboratory temperatures ranging from 15-25° C.; THF: tetrahydrofuran.

### Example 1: Exemplary Preparation of Rebastinib

Intermediate 1 (4-chloro-N-methylpicolinamide): To stirring anhydrous DMF (25 mL) was slowly added SOCl₂ (125 mL) at such a rate that the reaction temperature was maintained at 40-50° C. Pyridine-2-carboxylic acid (25 g, 0.2 mol) was added in portions over 30 min and the resulting mixture was heated at reflux for 16 h during which time a yellow solid precipitated. After cooling to RT, the mixture was diluted with toluene (80 mL) and concentrated. This process was repeated three times. The resulting dry residue was washed with toluene and dried under reduced pressure to yield 4-chloro-pyridine-2-carbonyl chloride (27.6 g, 79% yield), which was used in the next step without purification.

To a solution of 4-chloro-pyridine-2-carbonyl chloride (27.6 g, 0.16 mol) in anhydrous THF (100 mL) at 0° C. was added dropwise a solution of MeNH₂ in EtOH. The resulting mixture was stirred at 3° C. for 4 h. The reaction mixture was concentrated under reduced pressure to yield a solid, which was suspended in EtOAc and filtered. The filtrate was washed with brine (2×100 mL), dried and concentrated to yield 4-chloro-N-methylpicolinamide (16.4 g, 60% yield) as a yellow solid. ¹H NMR (400 MHz, DMSO-d₆) δ 8.78 (br s, 1H), 8.55 (d, J=5.2 Hz, 1H), 7.97 (d, J=2.0 Hz, 1H), 7.66 (m, 1H), 2.82 (d, J=4.8 Hz, 3H); MS (ESI) m/z: 171.0 (M+H⁺).

Intermediate 2: 4-(4-amino-3-fluorophenoxy)-N-methylpicolinamide): A solution of 4-amino-3-fluorophenol (2.00 g, 15.7 mmol) in anhydrous DMA (32 mL) was degassed by evacuation of the head space and backfilling with argon (repeated 3×). The solution was treated with potassium tert-butoxide (2.12 g, 18.9 mmol) and the resultant mixture was sonicated briefly to bring all solids into the solvent volume and was stirred at RT for 30 min. Intermediate 1 (2.68 g, 15.7 mmol) was added. The reaction mixture was degassed a second time and the reaction mixture was heated to 100° C. overnight under argon. The reaction mixture was poured into ethyl acetate (400 mL) and washed with water (3×100 mL) and saturated brine (2×100 mL). The combined aqueous was extracted with EtOAc (100 mL). The combined organics were dried (MgSO₄), concentrated in vacuo to a brown oil and purified by silica gel chromatography to provide 4-(4-amino-3-fluorophenoxy)-N-methylpicolinamide (3.18 g, 77% yield). ¹H NMR (400 MHz, DMSO-d₆) δ 8.76 (m, 1H), 8.48 (d, J=5.7 Hz, 1H), 7.36 (d, J=2.6 Hz, 1H), 7.10 (dd, J=5.7, 2.6 Hz, 1H), 7.02 (dd, J=11.8, 2.6 Hz, 1H), 6.86 (t, J=9.8 Hz, 1H), 6.79 (dd, J=8.9, 2.5 Hz, 1H), 5.23 (s, 2H), 2.79 (d, J=4.9 Hz, 3H); MS (ESI) m/z: 262.0 (M+H⁺).

Intermediate 3: (2,2,2-trichloroethyl 3-tert-butyl-1-(quinolin-6-yl)-1H-pyrazol-5-ylcarbamate): A solution of triflic anhydride (42.8 g, 0.15 mol) in CH₂Cl₂ (100 mL) was added dropwise to a 0° C. solution of 6-hydroxyquinoline (20.00 g, 0.138 mol) and pyridine (23 g, 0.277 mol) in CH₂Cl₂ (500 mL). The cooling bath was removed and the resulting solution was stirred at RT for 4 h. The reaction mixture was washed with water (3×300 mL) and the organic phase was dried (MgSO₄) and concentrated under vacuum to afford crude quinolin-6-yl trifluoromethanesulfonate (40 g, >100% yield) as an oil. ¹H-NMR (400 MHz, DMSO-d₆) δ 9.00 (d, 1H, J=2.8 Hz), 8.50 (d, 1H, J=8.0 Hz), 8.21 (d, J=2.8 Hz, 1H), 8.18 (d, J=9.2 Hz, 1H), 7.80 (m, 1H), 7.64 (m, 1H); MS (ESI) m/z: 277.9 (M+H⁺).

To a suspension of quinolin-6-yl trifluoromethanesulfonate (40 g, 0.14 mol), benzophenone hydrazone (35.6 g, 0.18 mol), cesium carbonate (74 g, 0.23 mol) and 1,1′-bis(diphenylphosphino)ferrocene (2.5 g, 4.5 mmol) in degassed toluene (1 L) was added palladium acetate (0.013 g, 0.058 mmol). The resultant mixture was heated to 90 degrees C. under a nitrogen atmosphere. After 16 h, the mixture was concentrated in vacuo and the residue was purified via silica gel column chromatography (EtOAc/pet ether) to provide 1-(diphenylmethylene)-2-(quinolin-6-yl)hydrazine (32 g, 68.6% yield). ¹H-NMR (300 MHz, DMSO-d.sub.6) δ 9.22 (s, 1H), 8.58 (t, J=1.8 Hz, 1H), 8.13 (d, J=3.6 Hz, 1H), 7.80 (d, J=3.6 Hz, 1H), 7.61 (d, J=3.9 Hz, 1H), 7.59-7.51 (m, 4H), 7.50 (d, J=3.6 Hz, 2H), 7.33-7.39 (m, 6H); MS (ESI) m/z: 324 (M+H⁺).

A solution of 1-(diphenylmethylene)-2-(quinolin-6-yl)hydrazine (32 g, 99 mmol) and 4,4-dimethyl-3-oxo-pentanenitrile (26 g, 0.15 mol) in ethanol (500 mL) was treated with conc. HCl (80 ml, 12N, 0.96 mol) and the mixture was heated to reflux overnight. The cooled reaction mixture was concentrated under vacuum and the residue was washed with Et₂O to remove the diphenylketone. The crude product was dissolved in EtOAc and neutralized (pH 8) with saturated Na₂CO₃ solution. The organic layer was dried (Na₂SO₄), concentrated in vacuo and purified by silica gel chromatography to give 5-tert-butyl-2-quinolin-6-yl-2H-pyrazol-3-ylamine (23 g, 87% yield). ¹H-NMR (300 MHz, DMSO-d₆) δ 8.86 (m, 1H), 8.39 (d, J=5.7 Hz, 1H), 8.11-8.02 (m, 3H), 7.54 (m, 1H), 5.46 (s, 1H), 5.42 (br s, 2H), 1.23 (s, 9H); MS (ESI) m/z: 267.2 (M+H⁺).

To a cold solution (−10° C.) of 5-tert-butyl-2-quinolin-6-yl-2H-pyrazol-3-ylamine (8.00 g, 30 mmol) in 100 ml of CH₂Cl₂ was added pyridine (8.0 ml, 99 mmol) and DMAP (100 mg), followed by a solution of trichloroethyl chloroformate (8.9 ml, 42 mmol) in 30 ml of CH₂Cl₂ over a period of 20 minutes. After stirring for 1 hour, water (100 ml) was added, stirring continued for 10 more minutes and the organic layer separated. The organic layer was washed with brine, dried and the dark brown residue obtained after removal of the solvent crystallized from acetonitrile to furnish 2,2,2-trichloroethyl 3-tert-butyl-1-(quinolin-6-yl)-1H-pyrazol-5-ylcarbamate as a white solid (8.23 g, 62% yield). ¹H NMR (DMSO-d₆) δ 10.15 (br s, 1H) 8.93 (m, 1H), 8.41 (d, J=8 Hz, 1H), 8.11 (m, 2H), 7.90 (dd, J=8, 2 Hz, 1H), 7.60 (dd, J=6.4, 4.2 Hz, 1H), 6.39 (s, 1H), 4.85 (s, 2H), 1.32 (s, 9H); MS (ESI) m/z: 442 (M+H⁺).

Rebastinib: A solution of Intermediate 3 (7.0 g, 15.8 mmol), Intermediate 2 (4.14 g, 15.8 mmol) and DIEA (4.5 g, 34.9 mmol) in DMSO (70 ml) was heated in an oil-bath at 70.degree. C. for 8 hrs. The reaction mixture was poured into water (500 ml), stirred overnight and the solids were separated by filtration. Successive crystallization of the crude product from toluene and acetone provided rebastinib as a white crystalline solid (4.06 g, 46% yield). ¹H NMR (DMSO-d₆) δ 8.90 (m, 2H), 8.79 (m, 1H), 8.52 (m, 2H), 8.2 (m, 3H), 7.96 (dd, J=9, 2 Hz, 1H), 7.63 (dd, J=8, 4 Hz, 1H), 7.40 (br s, 1H), 7.30 (dd, J=3, 12 Hz, 1H), 7.17 (m, 1H), 7.05 (d, J=9 Hz, 1H), 6.50 (s, 1H), 2.80 (d, J=5 Hz), 1.32 (s, 9H); MS (ESI) m/z: 554 (M+H⁺). The free base was treated with 0.1 M HCl to provide rebastinib bis hydrochloride salt as a pale yellow fluffy solid (2.40 g). ¹H NMR (DMSO-d₆) δ 9.56 (s, 1H), 9.26 (m, 2H), 9.10 (d, J=8 Hz, 1H), 8.85 (m, 1H), 8.55 (m, 2H), 8.46 (d, J=9 Hz, 1H), 8.33 (dd, J=9, 2 Hz, 1H), 8.11 (t, J=9 Hz, 1H), 8.03 (dd, dd, J=9, 2 Hz, 1H), 7.46 (d, J=3 Hz, 1H), 7.30 (dd, J=3, 12 Hz, 1H), 7.20 (dd, J=3, 6 Hz, 1H), 7.04 (brd, J=7 Hz, 1H), 6.49 (s, 1H), 2.80 (d, J=4.5 Hz), 1.33 (s, 9H).

### Example 2. A Clinical Study of Treating Advanced or Metastatic Tumors with Rebastinib and Paclitaxel

This is an open-label Phase 1b/2 multicenter study in patients with advanced or metastatic solid tumors where paclitaxel may be considered appropriate treatment. Rebastinib will be administered in combination with paclitaxel in repeated 28-day cycles to primarily assess the safety, tolerability and preliminary efficacy of the combination. Adverse events (AEs) will be assessed, and laboratory values, vital sign measurements, electrocardiograms (ECGs), ophthalmologic examinations, physical examinations and Eastern Cooperative Oncology Group (ECOG) Performance Status (PS) will be obtained to evaluate the safety and tolerability of rebastinib when administered in combination with paclitaxel. Pharmacokinetic (PK) and pharmacodynamic (PD) samples will be collected at pre-specified time points.

The study consists of two parts (Part 1 and Part 2). In Part 1, patients were assigned to one of two pre-defined dose levels of rebastinib (50 or 100 mg BID) in combination with paclitaxel administered by intravenous (IV) infusion at 80 mg/m². Paclitaxel is to be administered by IV infusion at 80 mg/m² over approximately 60 minutes on Day 1, Day 8 and Day 15 of repeated 28-day cycles. Rebastinib is to be administered orally prior to the paclitaxel infusion.

Each arm was planned to enroll at least 12 evaluable patients. Additionally, if rebastinib at 100 mg BID was deemed unsafe, an additional arm dosing at 75 mg BID of rebastinib in combination with paclitaxel may be initiated. Up to 36 evaluable patients were dosed in Part 1. Safety was continuously monitored, and any arm may be terminated early if deemed unsafe by the Sponsor.

Safety and tolerability, PK and PD, and preliminary efficacy data obtained in Part 1 was used to determine the RP2D. Data for at least 12 evaluable patients through Cycle 1 must be available in an arm to declare the dose as the RP2D. Patients must receive ≥80% of planned doses of rebastinib and paclitaxel in Cycle 1 to be considered as evaluable. Enrollment of patients was paused for determination of the RP2D prior to initiation of Part 2. An RP2D was chosen by the Sponsor in consultation with the Investigators.

Upon determination of the RP2D, Part 2 is initiated to dose up to 165 evaluable patients using the RP2D across five indication-specific cohorts. A Simon's two-stage design is applied to Part 2 to further evaluate the safety, tolerability, and preliminary efficacy of rebastinib in combination with paclitaxel in triple-negative breast (Cohort 1), inflammatory breast (Cohort 2), ovarian (Cohort 3), endometrial adenocarcinoma (Cohort 4), and gynecologic carcinosarcoma (Cohort 5) cancers. Each cohort initially enrolls up to 18 evaluable patients in the first stage. Patients must receive at least 1 dose of the combination and have 1 post-baseline assessment (including assessment of clinical progression) or be discontinued prior to the postbaseline disease assessment due to an AE at least possibly related to rebastinib, to be considered evaluable. Tumor response is assessed according to Response Evaluation Criteria in Solid Tumors (RECIST) Version 1.1. The decision to enroll patients beyond the first stage is based on response assessments obtained after the first post-dose response assessment of the last patient enrolled in the first stage of a cohort. If >4 responses (defined as partial response [PR] or complete response [CR]) are seen in a cohort, additional patients are enrolled for a total of up to 33 patients. If ≤4 responses are seen in a cohort, the cohort will be terminated. If >4 responses are seen prior to the last evaluable patient in the first stage, expanding the cohort may be triggered earlier. Enrollment in each cohort will pause between the first and second stage of the Simon two-stage for evaluation of response. Patients will receive study treatment until they develop progressive disease, experience unacceptable toxicity, or withdraw consent.

Rebastinib 50 and 100 mg BID in combination with weekly paclitaxel at 80 mg/m² was generally well tolerated in patients with advanced tumors. There was a partial response observed in 8 patients total: 5 and 3 at 50 and 100 mg BID, respectively. Responses were observed in heavily pre-treated patients, including prior therapy with paclitaxel. Exposure to rebastinib was dose proportional at the 50 mg BID and 100 mg BID doses when given in combination with paclitaxel. Mean circulating Ang-2 levels increased with exposure to higher doses of rebastinib in combination with paclitaxel, indicating TIE2 inhibition.

The data from Part 1 support further exploration of the combination in Part 2 with disease specific cohorts. Rebastinib at 100 mg BID in combination with weekly paclitaxel at 80 mg/m² was chosen as RP2D at the initiation of Part 2. However, a high incidence of muscular weakness with rebastinib at 100 mg BID in combination with weekly paclitaxel at 80 mg/m² in Part 2 required dose reduction of rebastinib to 50 mg BID with weekly paclitaxel at 80 mg/m², including the starting dose.

Part 1 included treatment of a patient with mesothelioma with 50 mg BID rebastinib and paclitaxel at 80 mg/m². The patient had two prior lines of treatment with carboplatin and alimta. At Cycle 4, the dose was reduced to 25 mg BID rebastinib and paclitaxel at 60 mg/m², with partial response reported at Cycle 8, Day 1.

### Triple Negative Breast Cancer (Cohort 1)

Patient responses in disease-specific cohorts in Part 2 were evaluated. In the triple negative breast cancer cohort (Cohort 1), one patient has been reported to achieve partial response at Cycle 8, Day 1 and one patient has been reported to achieve partial response at both Cycle 3, Day 1 and Cycle 5, Day 1. In Cohort 1, two patients have been reported to achieve stable disease at both Cycle 3, Day 1 and Cycle 5, Day 1, and one patient has been reported to achieve stable disease at both Cycle 3, Day 1.

### Inflammatory Breast Cancer (Cohort 2)

In the inflammatory breast cancer cohort (Cohort 2), two patients have been reported to achieve partial response at Cycle 3, Day 1, and one patient was reported to achieve partial response in Cycle 3, Day 1, Cycle 5, Day 1, and Cycle 8, Day 1. In Cohort 2, one patient was reported to achieve stable disease in both Cycle 3, Day 1 and Cycle 5, Day 1 and one patient was reported to achieve stable disease in Cycle 3, Day 1.

### Ovarian Cancer (Cohort 3)

### Summary of Clinical Results—Time Point 1

In the ovarian cancer cohort (Cohort 3), a summary of clinical results of the study are provided at different time points. At one time point, twelve patients were reported to achieve stable disease at Cycle 3, Day 1; of these twelve patients, three patients were reported to achieve partial response at Cycle 5, Day 1, four patients were reported to achieve stable disease at Cycle 5, Day 1, one patient was reported to achieve stable disease at Cycle 5, Day 8, two patients were reported to achieve stable disease at Cycle 8, Day 1, and one patient was reported to achieve a partial response at Cycle 5, Day 1. Two patients were reported to achieve a partial response in Cycle 3, Day 1; of these two patients, one patient was reported to achieve a partial response at Cycle 5, Day 1.

### Summary of Clinical Results—Time Point 2

At a second time point, 20 patients are enrolled; the median age is 58 years. All received at least 1 prior regimen with paclitaxel/carboplatin; the median number of prior therapies was 5 (2, 7). Ten patients were initially treated with 100 mg BID rebastinib (reduced to 50 mg BID due to muscular weakness [4 patients in this cohort]) and 10 patients with 50 mg BID rebastinib with a treatment median duration of 17.4 weeks (0.7, 31.8). In 17 evaluable patients, there were 5 partial responses and 9 stable diseases for an ORR of 29% and CBR of 82%. In addition, 7/15 (47%) pts had a CA-125 response. No additional muscular weakness was observed at reduced dose of 50 mg BID in this cohort.

### Summary of Clinical Results—Time Point 3

29 patients with ovarian cancer were enrolled in this portion of the study as a safety population. Patients were treated with rebastinib (50 or 100 mg BID) in combination with 80 mg/m² intravenous weekly paclitaxel (day 1, day 8, and day 15 of repeated 28-day cycles). 10 of the patients treated with rebastinib were at a starting dose of 100 mg BID and 80 mg/m² weekly paclitaxel, of which the doses of 7 were reduced to 50 mg BID and 80 mg/m² weekly paclitaxel, and 28 patients were at a starting dose of 50 mg BID and 80 mg/m² weekly paclitaxel. 23 (79%) patients received ≥4 prior anticancer regimens, and all patients received prior platinum and taxane-based therapy; 89% received anti-angiogenic therapy, 62% received anti-PARP therapy, and 31% received immunotherapy.

In the safety population, the patients received a starting dose of rebastinib at 100 mg BID (n=7), dose-reduced rebastinib from 100 to 50 mg BID (n=3), or had a starting dose rebastinib 50 mg BID (n=19). 24 of the 29 patients met the modified intention-to-treat (mITT) criteria. Anti-tumor activity was assessed using RECIST v1.1 and Gynecological Cancer Intergroup CA-125 response. From 24 patients in the mITT population, the objective response rate was 38% and the clinical benefit rate at 8 weeks was 88%. From 24 evaluable patients, 17 had CA-125 measurements and 10 (59%) had a CA-125 response.

### Summary of Clinical Results—Time Point 4

38 patients in a platinum-resistant ovarian cancer cohort were enrolled in this portion of the study as a safety population. Patients were treated with rebastinib (50 or 100 mg BID) in combination with 80 mg/m² intravenous weekly paclitaxel (day 1, day 8, and day 15 of repeated 28-day cycles). 34 of the 38 patients met the met the modified intention-to-treat (mITT) criteria. 27 (71%) patients received ≥4 prior anticancer regimens, and 97% patients received prior platinum and taxane-based therapy; 84% received bevacizumab, 66% received a PARP inhibitor, 31% received immunotherapy, and the average number of lines of therapy after platinum resistance was 2.

The objective response rate was 35% (confirmed+unconfirmed) and the clinical benefit rate at 8 weeks was 88% in the 34 patients in the mITT population. A CA-125 response occurred in 17 of 26 evaluable patients (65.4%). Table 1 summarizes efficacy outcomes of the 34 patients in the mITT population at this time point.

TABLE 1

| | Total |
|---|---|
| Outcome | (N = 34) |
| Best Overall Response | |
| Partial Response | 12 (35.3%) |
| Confirmed Partial Response | 5 (14.7%) |
| Single Response Awaiting Confirmation | 4 (11.8%) |
| Unconfirmed Single Response | 3 (8.8%) |
| Stable Disease | 18 (52.9%) |
| Progressive Disease | 1 (2.9%) |
| Not Evaluable | 3 (8.8%) |
| Time to Response | |
| n | 12 |
| Mean (SD) | 3.1 (1.3) |
| Median [range] | 3.4 [1.7, 6.4] |
| Duration of Response | |
| n | 12 |
| Events (%) | 5 (41.7%) |
| Kaplan-Meier Median (80% CI) | 5.5 (1.9, NE) |
| Range | 0.03+, 5.6 |
| Clinical Benefit Ratio at 16 weeks | 18 of 27 (66.7%) |

NE: Not evaluated.

### Endometrial Cancer (Cohort 4)

### Summary of Clinical Results—Time Point 1

In the endometrial adenocarcinoma cohort (Cohort 4), a summary of clinical results of the study are provided at different time points. At one time point, four patients have been reported to achieve partial response at Cycle 3, Day 1; of these four patients, two patients were reported to achieve a partial response in Cycle 5, Day 1 and two patients were reported to achieve a partial response in both Cycle 5, Day 1 and Cycle 8, Day 1. Eight patients have been reported to achieve stable disease at Cycle 3, Day 1; of these eight patients, four patients have been reported to achieve stable disease at Cycle 5, Day 1, one patient was reported to achieve partial response at Cycle 5, Day 1, and one patient was reported to achieve stable disease at Cycle 5, Day 1 and partial response at Cycle 8, Day 1.

### Summary of Clinical Results—Time Point 2

At a second time point, 21 patients were reported to complete at least Cycle 1, Day 1 in Cohort 4. The median age is 66 years, and all received at least 1 prior line of paclitaxel/carboplatin, and 20 (95%) received at least 2 prior anti-cancer therapies.

21 patients were enrolled and completed at least Cycle 1, Day 1 (C1D1); 18 of which had post baseline measurements. 16 of the patients treated with rebastinib were at a starting dose of 100 mg BID and 80 mg/m² weekly paclitaxel, of which the doses of 11 were reduced to 50 mg BID and 80 mg/m² weekly paclitaxel, and 5 patients were at a starting dose of 50 mg BID and 80 mg/m² weekly paclitaxel. Of the 21 patients who completed at least Cycle 1, Day 1, the median age was 66 years, all received at least 1 prior line of paclitaxel/carboplatin, and 20 (95%) received at least 2 prior anti-cancer therapies. Of the 21 patients who completed at least Cycle 1, Day 1, the median duration of treatment was 16 weeks. Of the 21 patients, 18 were determined to be efficacy evaluable patients (mITT). From these 18 evaluable patients, there were 7 partial response (PR) and 6 stable disease (SD) determinations for an ORR of 39% and a CBR of 72% at 8 weeks.

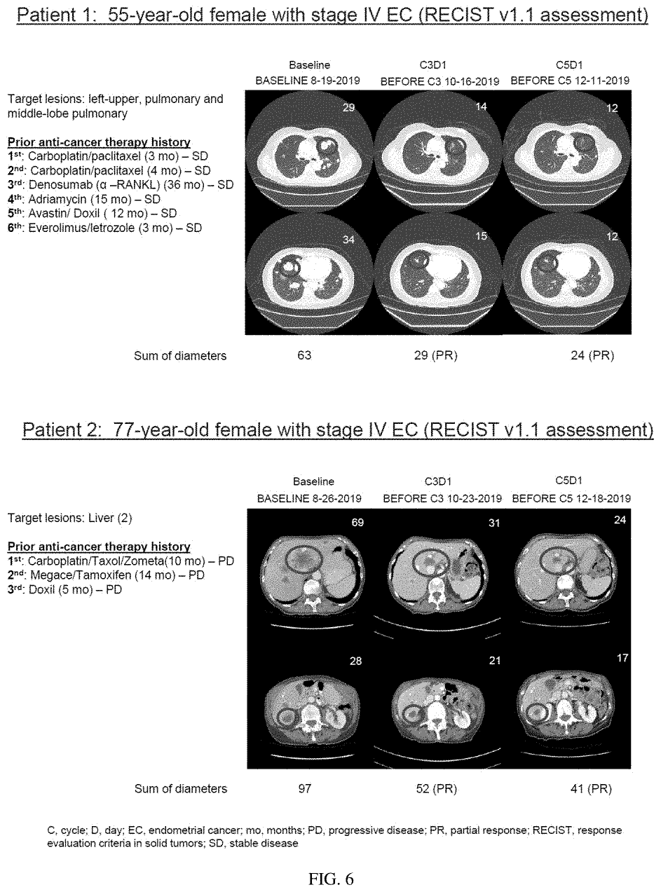

FIG. 6 depicts RECIST v1.1 assessments of two stage IV endometrial cancer patients with corresponding anticancer history listed. Sum of diameters across indicated target lesions are depicted with respect to measurements at baseline, Cycle 3, Day 1, and Cycle 5, Day 1.

### Summary of Clinical Results—Time Point 3

At a third time point, 38 patients with endometrial cancer were enrolled in this portion of the study as a safety population. 16 of the patients treated with rebastinib were at a starting dose of 100 mg BID and 80 mg/m² weekly paclitaxel, of which the doses of 11 were reduced to 50 mg BID and 80 mg/m² weekly paclitaxel, and 22 patients were at a starting dose of 50 mg BID and 80 mg/m² weekly paclitaxel. 33 of the 38 patients met the met the modified intention-to-treat (mITT) criteria.

All patients received ≥1 prior line of the combination of paclitaxel/carboplatin and 20 (95%) received ≥2 prior anti-cancer regimens including multiple regimens of paclitaxel. 40% received prior anti-angiogenesis therapy (mostly bevacizumab). 29% received hormonal therapy including 55% (6 of 11) of patients reported to achieve a partial response. 3 patients received Lenvatinib/pembrolizumab, of which 2 achieved partial response and 1 achieved stable disease.

From 33 patients in the mITT population, the objective response rate (ORR) was 33% and the clinical benefit rate at 16 weeks was 53%.

Table 2 summarizes efficacy outcomes of the 33 patients in the mITT population at this time point.

TABLE 2

| | Total |
|---|---|
| Efficacy Outcome | (N = 33) |
| Best Overall Response | |
| Partial Response | 11 (33.3%) |
| Confirmed Partial Response | 8 (24.2%) |
| Single Response Awaiting Confirmation | 1 (3.0%) |
| Unconfirmed Single Response | 2 (6.1%) |
| Stable Disease | 11 (33.3%) |
| Progressive Disease | 6 (18.2%) |
| Not Evaluable: discontinued prior to | 5 (15.2%) |
| radiological assessment | |
| Time to Response | |
| n | 11 |
| Mean (SD) | 3.3 (1.7) |
| Median [range] | 3.5 [1.6, 6.4] |
| Duration of Response | |
| n | 11 |
| Events (%) | 3 (27.3) |
| Kaplan-Meier Median (80% CI) | 7.4 (7.4, NE) |
| Range | 0.03+, 10.2 |
| Clinical Benefit Ratio at 16 weeks | 17/32 (53.1) |

NE: Not evaluated.

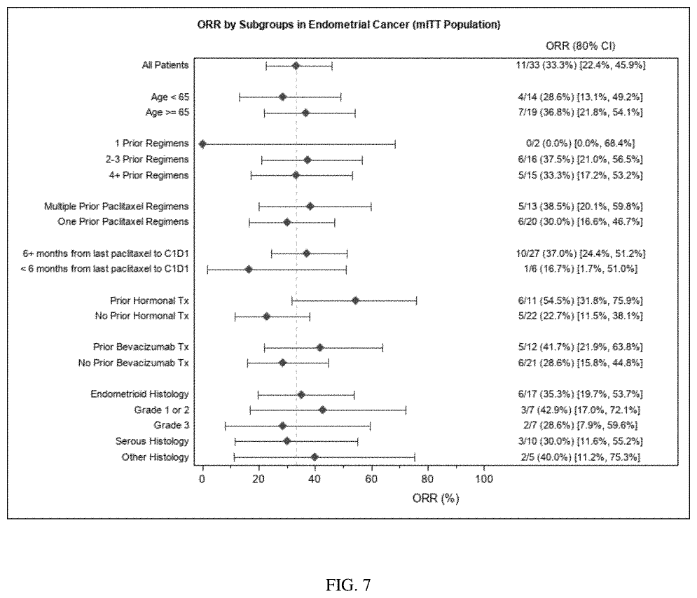

FIG. 7 shows objective response rate (ORR) data for the mITT population by Subgroup Analyses. The ORR was consistent across subgroups defined by age, number of prior regimens, number of prior paclitaxel regimens, and histology.

### Summary of Clinical Results—Time Point 4

At a fourth time point, 38 patients with endometrial cancer were enrolled in this portion of the study as a safety population. 16 of the patients treated with rebastinib were at a starting dose of 100 mg BID and 80 mg/m² weekly paclitaxel, of which 11 were reduced to 50 mg BID and 80 mg/m² weekly paclitaxel. From 33 evaluable patients, ORR was 33% and clinical benefit at 8 and 16 weeks was 70% and 54%, respectively including 11 PRs (8 confirmed) and 12 SD. At this time point, the median follow up was 4.4 months. The KM median duration of response was 7.4 months (90% CI: 2.7, NE) with median PFS of 6.2 months (90% CI: 3.7, 9.4) and. Treatment-emergent adverse events (>20% mostly ≤grade 2) included, fatigue (n=18), constipation, peripheral edema (each n=16), peripheral sensory neuropathy, nausea (each n=15), dyspnea (n=13), alopecia, hypokalemia (each at n=11), diarrhea, hypomagnesemia (each n=10) dry mouth, dysgeusia (each at n=9), arthralgia, hypertension, dehydration, GERD and muscular weakness (each at n=8). Serious adverse events (SAE) related (or possibly) to rebastinib included reversible muscular weakness (n=2 at 100, n=1 at 50 mg BID), nausea (n=2), acute myocardial infarction, angina pectoris, atrial flutter, constipation, dehydration, dyspnea, fatigue, non-infective encephalitis, peri tonsilitis, stress cardiomyopathy and UTI (each at n=1) and were resolved after dose interruption.

### Gynecologic Carcinosarcoma (Cohort 5)

In the gynecologic carcinosarcoma cohort (Cohort 5), one patient was reported to achieve a partial response on Cycle 5, Day 1.

### Example 3. A Clinical Study of Treating Breast Cancer Patients, Ovarian Cancer Patients, and Mesothelioma Patients with Rebastinib and Carboplatin

This is a Phase 1b/2 study where patients with mesothelioma are administered rebastinib in combination with carboplatin. The study follows a Simon 2-stage design at the MTD and R2PD. Safety and tolerability, PK and PD, and preliminary efficacy data was used to determine the RP2D. Upon determination of the RP2D or MTD, the Simon 2-stage design is initiated to dose evaluable patients using the RP2D or MTD across three indication-specific cohorts (breast cancer, ovarian cancer, and mesothelioma).

Each cohort initially enrolls up to 18 evaluable patients in the first stage. Patients must receive at least 1 dose of the combination and have 1 post-baseline assessment (including assessment of clinical progression), or be discontinued prior to the postbaseline disease assessment due to an AE at least possibly related to rebastinib, to be considered evaluable. Tumor response is assessed according to Response Evaluation Criteria in Solid Tumors (RECIST) Version 1.1. The decision to enroll patients beyond the first stage is based on response assessments obtained after the first post-dose response assessment of the last patient enrolled in the first stage of a cohort. If >4 responses (defined as partial response [PR] or complete response [CR]) are seen in a cohort, additional patients are enrolled for a total of up to 33 patients. If ≤4 responses are seen in a cohort, the cohort will be terminated. If >4 responses are seen prior to the last evaluable patient in the first stage, expanding the cohort may be triggered earlier. Enrollment in each cohort will pause between the first and second stage of the Simon two-stage for evaluation of response. Patients will receive study treatment until they develop progressive disease, experience unacceptable toxicity, or withdraw consent.

### Summary of Clinical Results—Time Point 1

A summary of clinical results of the study are provided at different time points. At one time point in the study, rebastinib at 50 mg and 100 mg BID in combination with carboplatin AUC5 were tolerated and rebastinib at 100 mg BID in combination with carboplatin AUC6 was tolerated (3 patients). No muscular weakness was reported with a rebastinib and carboplatin combination. No response has been seen in the dose escalation cohorts. The mesothelioma disease-specific cohort was initiated with rebastinib at 100 mg BID in combination with carboplatin AUC5.

### Summary of Clinical Results—Time Point 2

At a second time point, 22 patients were enrolled in the study (3 cohorts: 3 patients at rebastinib 50 mg BID+carboplatin AUC5, 14 patients at rebastinib 100 mg BID+carboplatin AUC5, and 5 pts at rebastinib 100 mg BID+carboplatin AUC6). The median age was 61 yrs. Most frequent diagnoses were breast cancer (n=5); neuroendocrine carcinoma (n=3); pancreatic adenocarcinoma, NSCLC, and cholangiocarcinoma (n=2 each). The median number of prior anti-cancer therapies was 4 (1, 12). Median duration of treatment was 8.4 weeks (0.9, 22.6).

Preliminary clinical activity included 1 partial response (PR), 10 stable disease (SD) and a CBR of 52% at 6 weeks. Rebastinib exposure was dose proportional. Rebastinib 100 mg BID with carboplatin AUC5 was selected as recommended phase 2 dose (RP2D). An increased frequency of muscular weakness in the dose expansion cohort was observed at this dose and as a result the RP2D was adjusted to rebastinib 50 mg BID with carboplatin AUC5.

### Example 4. A Preclinical Study of the Combination Efficacy of Rebastinib and Carboplatin in a Mesothelioma Model

This study was conducted to obtain preclinical evidence of rebastinib/carboplatin combination efficacy in mesothelioma to support clinical development. The study was an in vivo evaluation of rebastinib single agent and in combination with carboplatin (SOC), in Low Passage Champions TUMORGRAFT® Models of Human Mesothelioma in immunocompromised mice.

Mesothelioma PDx models were studied each with 8 animals per group for total of 4 groups per model. The study endpoint was when the mean tumor volume of the control reaches 1500 mm³ or 60 days max. Terminal tumors were be collected, fixed, and embedded.

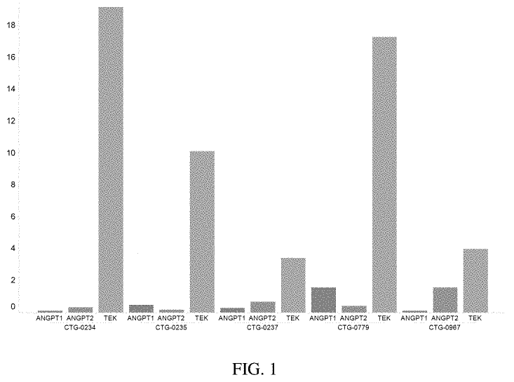

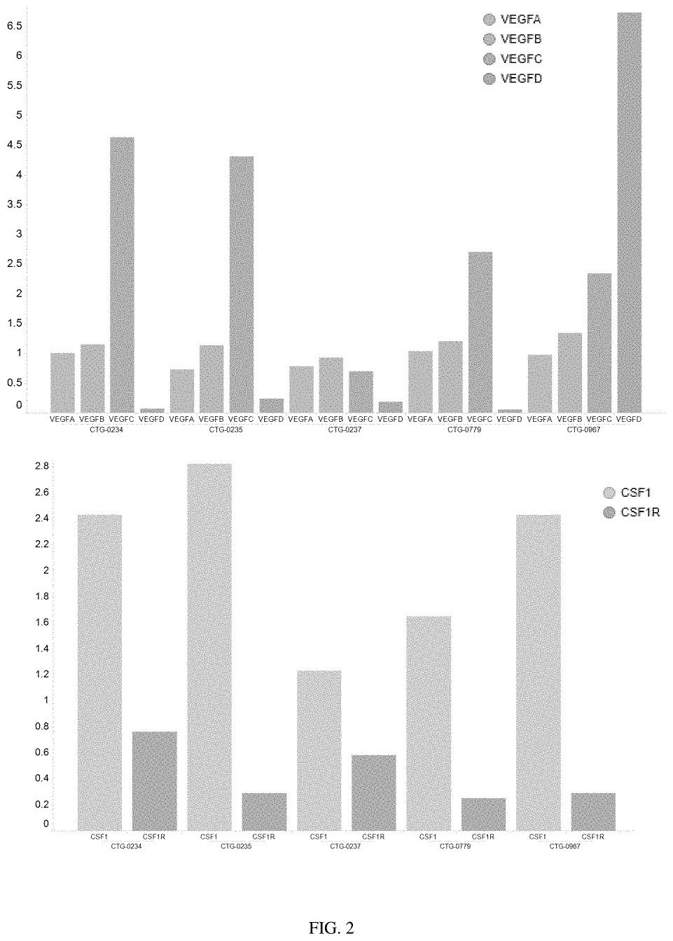

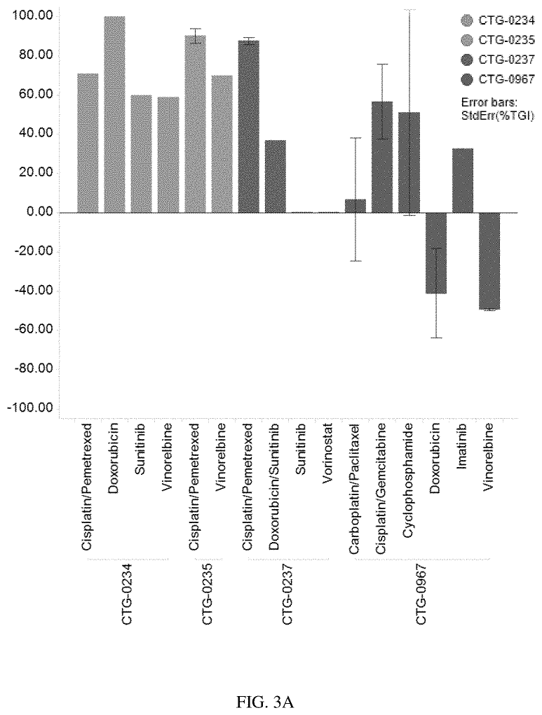

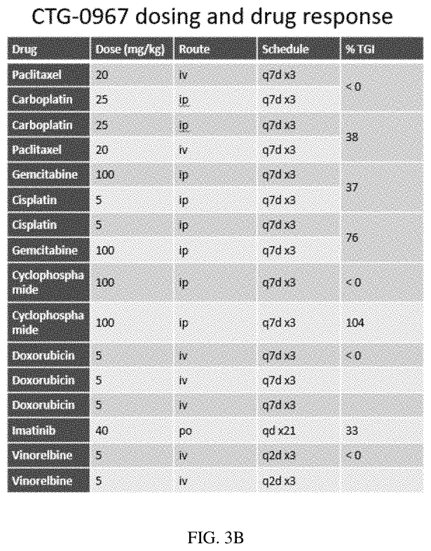

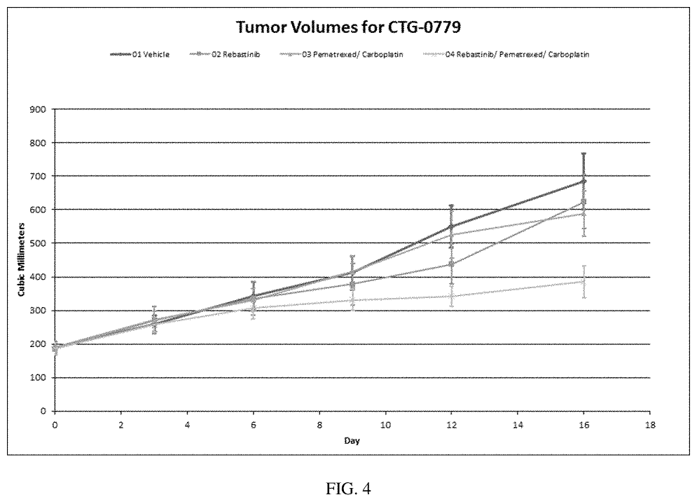

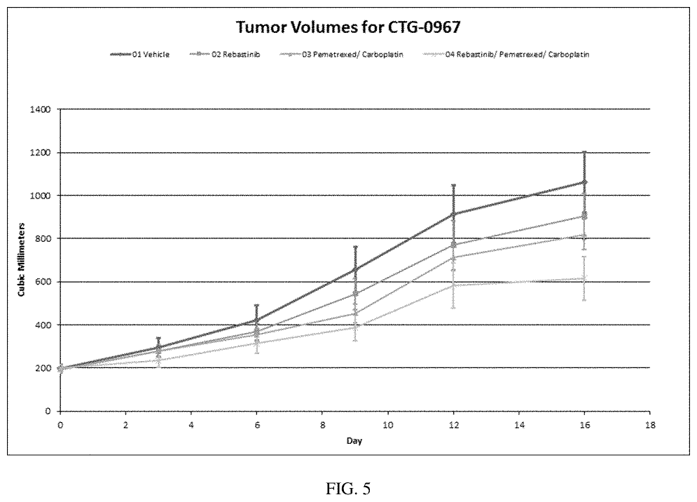

FIG. 1 shows the TEK/ANG1/Ang2 gene expression in models evaluated. FIG. 2 shows the VEGF/CSF1/CSF1R gene expression in models evaluated. FIG. 3A shows the tumor growth inhibition in the exemplary evaluated models using various dosing of drugs and drug combinations further described in FIG. 3B. FIG. 4 and FIG. 5 demonstrate tumor volume changes in Models 1 and 2 after administering vehicle, rebastinib, a combination of carboplatin and pemetrexed, and a combination of rebastinib, pemetrexed, and carboplatin. For example, a combination of 25 mg/kg rebastinib administered via intraperitoneal administration and 20 mg/kg carboplatin administered intravenously demonstrated a 38% tumor inhibition in Model 2.

### Example 5. A Clinical Study of Treating Endometrial Cancer Patients with Rebastinib and Paclitaxel

This is a Phase 3 multicenter study in patients with advanced endometrial cancer who have received at least 2 and no more than 3 lines of prior systemic therapy where a combination therapy of rebastinib and paclitaxel may be considered appropriate treatment.

Rebastinib will be administered in combination with paclitaxel to primarily assess the efficacy such as progression-free survival (PFS) and objective response rate (ORR) by independent radiologic review and overall survival (OS) of rebastinib and paclitaxel combination as compared to placebo and paclitaxel combination. Other parameters of efficacy, including but not limited to the clinical benefit rate (CBR), duration of response (DOR), and time to response will also be assessed, in addition to safety and tolerability of rebastinib and paclitaxel combination. In addition, patient-reported outcome (PRO) parameters of improvement of disease-related symptoms and quality of life (QOL) will be obtained as measured by Endometrial Cancer Module of the EORTC QLQ-EN24, European Organisation for Research and Treatment of Cancer (EORTC) Quality of Life Questionnaire (QLQ)-C30, EuroQol 5 Dimension 5 Level Scale (EQ-5D-5L), and GP5 question (“treatment bother”) from the Functional Assessment of Chronic Illness Therapy (FACIT). Additionally, frequency of treatment-emergent adverse events (TEAEs), serious adverse events (SAEs), adverse events of special interest (AESIs), dose modifications, interruptions, or discontinuation of study drug due to a TEAE, laboratory evaluations, physical examinations, electrocardiogram (ECGs), and vital signs will be obtained to evaluate the safety and tolerability of rebastinib when administered in combination with paclitaxel. Pharmacokinetic (PK) samples will be collected at pre-specified time points.

Rebastinib and matching placebo will be provided as 25 mg tablets for oral administration at 50 mg BID (approximately 12 hours apart with minimum of 6 hours between doses) on 28-day cycles. Paclitaxel will be administered in the clinic by intravenous (IV) infusion at 80 mg/m2 over approximately 60 minutes on Days 1, 8, and 15 in a 28-day cycle. Oral administration of rebastinib or placebo shall occur prior to the initiation of paclitaxel infusion. Premedication required by patients prior to paclitaxel infusion shall be administered per institutional practice before or after oral administration of rebastinib or placebo. In the event paclitaxel retreatment criteria (absolute neutrophil count ≥1000/μL and platelets ≥75,000/μL) are not met resulting in missed paclitaxel dosing on Days 1, 8, or 15, a single dose of paclitaxel may be administered on Day 22 of the 28-day cycle. For patients who are unable to tolerate paclitaxel, infusions may be stopped and patient will be discontinued from the study. Each arm is planned to enroll about 135 patients. Patients will receive study treatment until disease progression, unacceptable toxicity, or withdrawal of consent.

Study drugs may be interrupted or modified (i.e., dose reduced) at the discretion of the Investigator at any time due to AE, to accommodate palliative treatment, or for other reasons after consultation with the Sponsor. The causal relationship of each AE should be assessed in relation to rebastinib and to paclitaxel separately so that dose modifications can be made accordingly. Rebastinib should be interrupted for treatment-related Grade 3 AEs of any duration and can be resumed at the same or lower dose level when the toxicity has recovered to ≤Grade 1 or baseline. Oral study treatment may be dose reduced to 25 mg BID. If it is known or suspected that a patient cannot tolerate oral study treatment at 25 mg BID, then the patient must discontinue from study treatment (i.e., both treatments: oral and paclitaxel). Paclitaxel should be interrupted for treatment-related Grade 3 AEs of any duration and can be resumed at the same or lower dose level when the toxicity has recovered to ≤Grade 1 or baseline. Dose modifications are allowed per approved package insert or institutional guidelines. Paclitaxel dosing maybe reduced initially to 70 mg/m2 followed by another reduction to 60 mg/m2 if the initial reduction is not tolerable. A patient will be discontinued from paclitaxel if further dose reduction is needed.

Permanent discontinuation of rebastinib or paclitaxel should be considered for any treatment-emergent severe or life-threatening event. Upon experiencing any of the following conditions, a patient should discontinue study treatment permanently: any Grade 4 AE excluding hematological AEs or requirement for more than 2 dose reductions from the assigned dose at enrollment to manage AEs. For rebastinib, patient should discontinue study treatment if there is a treatment interruption for >14 days due to treatment-related AEs, and for paclitaxel, patient should discontinue study treatment if there is a treatment interruption for >21 days due to treatment-related AEs.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A method of treating a gynecologic carcinosarcoma in a patient that has previously received at least one line of treatment for the gynecologic carcinosarcoma, and is in need thereof, comprising administering to the patient an effective amount of the compound of Formula (I): 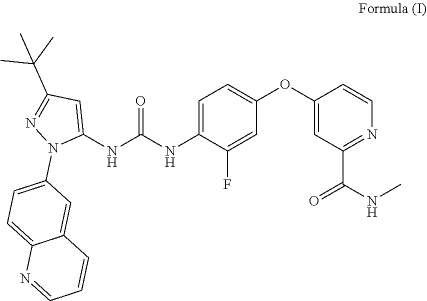 [SMILES: CNC(=O)c1cc(Oc2ccc(NC(=O)Nc3cc(C(C)(C)C)nn3-c3ccc4ncccc4c3)c(F)c2)ccn1]
or a pharmaceutically acceptable salt thereof.

2. A method of treating a gynecologic carcinosarcoma in a patient that has previously received at least one line of treatment for the gynecologic carcinosarcoma, and is in need thereof, comprising administering to the patient an effective amount of:
a) the compound of Formula (I) 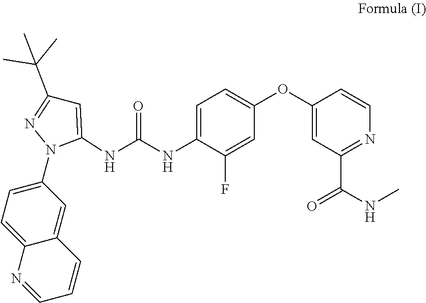 [SMILES: CNC(=O)c1cc(Oc2ccc(NC(=O)Nc3cc(C(C)(C)C)nn3-c3ccc4ncccc4c3)c(F)c2)ccn1]
or a pharmaceutically acceptable salt thereof; and
b) paclitaxel.

3. The method of claim 2, wherein after 4 to 6 cycles of treatment with the Compound of Formula (I) and the paclitaxel, or after occurrence of toxicity, the method further comprises administering to the patient an effective amount of the compound of Formula (I) or pharmaceutically acceptable salt thereof until disease progression or toxicity in the patient occurs.

4. The method of any one of claims 1-3, wherein the gynecologic carcinosarcoma is advanced gynecologic carcinosarcoma.

5. The method of any one of claims 1-4, further comprising histologically determining if the gynecologic carcinosarcoma is advanced, persistent or recurrent.

6. The method of any one of claims 1-5, wherein the at least one line of treatment is selected from the group consisting of a chemotherapy treatment, a checkpoint inhibitor treatment, a tyrosine-kinase inhibitor treatment, and a PARP-inhibitor treatment.

7. The method of claim 6, wherein the chemotherapy treatment comprises a chemotherapeutic agent selected from the group consisting of paclitaxel, paclitaxel protein-bound particles for injectable suspension, eribulin, docetaxel, ixabepilone, vincristine, vinorelbine, cisplatin, carboplatin, oxaliplatin, cyclophosphamide, ifosfamide, temozolomide, doxorubicin, pegylated liposomal doxorubicin, daunorubicin, idarubicin, and epirubicin, 5-fluorouracil, capecitabine, cytarabine, decitabine, 5-aza cytadine, gemcitabine, and methotrexate.

8. The method of claim 6, wherein the checkpoint inhibitor treatment comprises an anti-PD1 therapeutic or an anti-PD-L1 therapeutic.

9. The method of claim 6, wherein the tyrosine-kinase inhibitor treatment comprises a tyrosine kinase inhibitor selected from the group consisting of afatinib, axitinib, binimetinib, bosutinib, cabozantinib, cediranib, ceritinib, cobimetinib, crizotinib, dabrafenib, dasatinib, DCC-3014, erlotinib, everolimus, gefitinib, ibrutinib, imatinib, lapatinib, lenvatinib, lestaurtinib, nilotinib, nintedanib, palbociclib, pazopanib, ponatinib, regorafenib, ruxolitinib, semananib, sirolimus, sorafenib, sunitinib, temsirolimus, tofacitinib, trametinib, vandetanib, and vemurafenib.

10. The method of claim 6, wherein the PARP-inhibitor treatment comprises a PARP inhibitor selected from the group consisting of neraparib, olaparib, and rucaparib.

11. The method of claim 6 or claim 7, wherein the chemotherapy treatment comprises paclitaxel and/or carboplatin.

12. The method of any one of claims 1-11, wherein the gynecologic carcinosarcoma is of a homologous type or heterologous type.

13. The method of any one of claims 1-12, wherein after 28 days or more of administering the compound of Formula (I) or pharmaceutically acceptable salt to the patient, the patient has achieved a partial response condition or a stable disease condition.

14. The method of any one of claims 1-13, wherein the gynecologic carcinosarcoma is an endometrial carcinosarcoma.

15. The method of any one of claims 1-14, wherein the gynecologic carcinosarcoma is an ovarian carcinosarcoma.

16. The method of any one of claims 1-15, wherein the gynecologic carcinosarcoma is a vaginal carcinosarcoma.

17. The method of any one of claims 1-16, wherein the gynecologic carcinosarcoma is a cervical carcinosarcoma.

18. The method of any one of claims 1-17, comprising administering to the patient about 100 mg of the compound of Formula (I) or pharmaceutically acceptable salt thereof per day.

19. The method of any one of claims 1-17, comprising administering to the patient about 150 mg of the compound of Formula (I) or pharmaceutically acceptable salt thereof per day.

20. The method of any one of claims 1-17, comprising administering to the patient about 200 mg of the compound of Formula (I) or pharmaceutically acceptable salt thereof per day.

21. The method of any one of claims 1-20, wherein the compound of Formula (I) or pharmaceutically acceptable salt thereof is orally administered to the patient.

22. The method of any one of claims 2-21, wherein the paclitaxel is intravenously administered to the patient.

23. The method of any one of claims 2-22, wherein the paclitaxel is administered to the patient once per week.

24. The method of any one of claims 2-23, wherein the paclitaxel is administered to the patient once every three weeks.

25. The method of any one of claims 2-24, wherein the paclitaxel is administered to the patient by infusion once per week.

26. The method of any one of claims 2-25, wherein the paclitaxel is administered to the patient by infusion once every three weeks.

27. A method of treating endometrial adenocarcinoma in a patient in need thereof, comprising administering to the patient an effective amount of the compound of Formula (I) 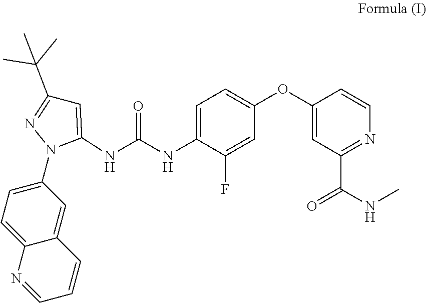 [SMILES: CNC(=O)c1cc(Oc2ccc(NC(=O)Nc3cc(C(C)(C)C)nn3-c3ccc4ncccc4c3)c(F)c2)ccn1]
or a pharmaceutically acceptable salt thereof.

28. A method for achieving at least a partial response in a patient having endometrial adenocarcinoma, comprising administering to the patient an effective amount of the compound of Formula (I) 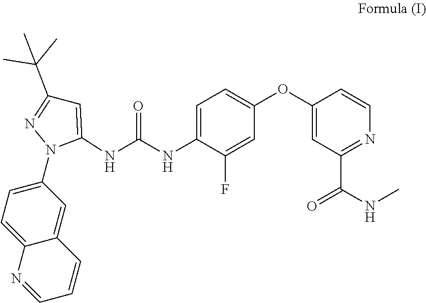 [SMILES: CNC(=O)c1cc(Oc2ccc(NC(=O)Nc3cc(C(C)(C)C)nn3-c3ccc4ncccc4c3)c(F)c2)ccn1]
or a pharmaceutically acceptable salt thereof.

29. A method for achieving a stable disease condition in a patient having endometrial adenocarcinoma, comprising administering to the patient an effective amount of the compound of Formula (I) 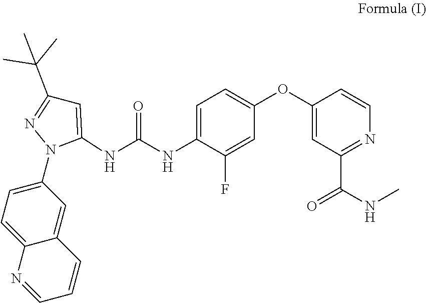 [SMILES: CNC(=O)c1cc(Oc2ccc(NC(=O)Nc3cc(C(C)(C)C)nn3-c3ccc4ncccc4c3)c(F)c2)ccn1]
or a pharmaceutically acceptable salt thereof.

30. The method of any one of claims 27-29, wherein the patient has previously been administered at least one prior line of platinum-based therapy.

31. The method of any one of claims 27-30, wherein after 28 days or more of administering the compound of Formula (I) or pharmaceutically acceptable salt thereof to the patient, the patient has at least a partial response or a stable disease condition.

32. The method of any one of claims 27-31, comprising administering to the patient about 100 mg of Formula (I) or pharmaceutically acceptable salt thereof per day.

33. The method of any one of claims 27-31, comprising administering to the patient about 200 mg of the compound of Formula (I) or pharmaceutically acceptable salt thereof per day.

34. The method of any one of claims 27-31, comprising administering to the patient about 150 mg of the compound of Formula (I) or pharmaceutically acceptable salt thereof per day.

35. The method of any one of claims 27-31, comprising administering to the patient about 50 mg of the compound of Formula (I) or pharmaceutically acceptable salt thereof twice per day.

36. The method of any one of claims 27-31, comprising administering to the patient about 100 mg of the compound of Formula (I) or pharmaceutically acceptable salt thereof twice per day.

37. The method of any one of claims 27-36, further comprising administering paclitaxel to the patient.

38. The method of any one of claims 27-37, wherein the compound of Formula (I) or pharmaceutically acceptable salt thereof is orally administered to the patient.

39. The method of claim 37 or claim 38, wherein the paclitaxel is intravenously administered to the patient.

40. The method of any one of claims 37-39, wherein the paclitaxel is administered to the patient once per week.

41. The method of any one of claims 37-39, wherein the paclitaxel is administered to the patient once every three weeks.

42. The method of any one of claims 37-40, wherein the paclitaxel is administered to the patient by infusion once per week.

43. The method of any one of claims 37-39 or claim 41, wherein the paclitaxel is administered to the patient by infusion once every three weeks.

44. A method of treating mesothelioma in a patient in need thereof, comprising administering to the patient about 50 mg to about 200 mg of the compound of Formula (I) 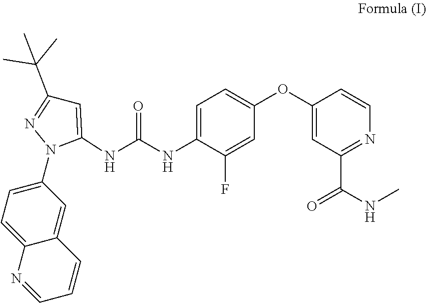 [SMILES: CNC(=O)c1cc(Oc2ccc(NC(=O)Nc3cc(C(C)(C)C)nn3-c3ccc4ncccc4c3)c(F)c2)ccn1]
or a pharmaceutically acceptable salt thereof per day.

45. The method of claim 44, comprising administering to the patient about 100 mg of the compound of Formula (I) or pharmaceutically acceptable salt thereof per day.

46. The method of claim 44, comprising administering to the patient about 150 mg of the compound of Formula (I) or pharmaceutically acceptable salt thereof per day.

47. The method of claim 44, comprising administering to the patient about 200 mg of Formula (I) or pharmaceutically acceptable salt thereof per day.

48. The method of claim 44 or claim 45, comprising administering to the patient about 50 mg of the compound of Formula (I) or pharmaceutically acceptable salt thereof twice per day.

49. The method of claim 44 or claim 47, comprising administering to the patient about 100 mg of the compound of Formula (I) or pharmaceutically acceptable salt thereof twice per day.

50. The method of any one of claims 44-49, further comprising administering a combination agent to the patient.

51. The method of claim 50, wherein the combination agent is carboplatin.

52. The method of claim 50, wherein the combination agent is paclitaxel.

53. The method of any one of claims 44-52, further comprising administering pemetrexed to the patient.

54. The method of any one of claims 44-53, wherein the patient has previously been administered at least one prior line of therapy.

55. The method of any one of claims 44-54, wherein the at least one prior line of therapy is a platinum-based therapy.

56. The method of any one of claims 44-55, wherein the patient has previously been administered carboplatin and/or pemetrexed.

57. The method of any one of claims 44-56, wherein the patient has previously been administered cisplatin and/or pemetrexed.

58. The method of any one of claims 44-57, wherein the compound of Formula (I) or pharmaceutically acceptable salt thereof is orally administered to the patient.

59. The method of any one of claims 44-58, wherein the combination agent is intravenously administered to the patient.

60. The method of any one of claims 44-59, wherein the combination agent is intravenously administered to the patient in an amount sufficient to provide an area under the curve of about 5 to about 7.

61. The method of any one of claims 44-60, comprising reducing each of the amount of the compound of Formula (I) or pharmaceutically acceptable salt thereof and the combination agent administered to the patient after one or more months of administering to the patient.

62. A method for achieving at least a partial response in a patient having mesothelioma, comprising administering to the patient an effective amount of the compound of Formula (I) 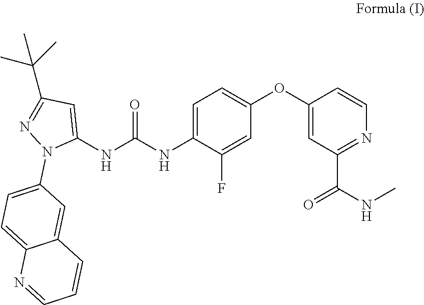 [SMILES: CNC(=O)c1cc(Oc2ccc(NC(=O)Nc3cc(C(C)(C)C)nn3-c3ccc4ncccc4c3)c(F)c2)ccn1]
or a pharmaceutically acceptable salt thereof.

63. A method for achieving a stable disease condition in a patient having mesothelioma, comprising administering to the patient the compound of Formula (I) 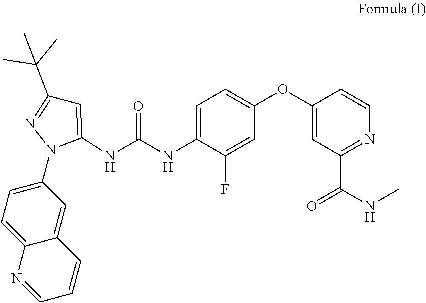 [SMILES: CNC(=O)c1cc(Oc2ccc(NC(=O)Nc3cc(C(C)(C)C)nn3-c3ccc4ncccc4c3)c(F)c2)ccn1]
or a pharmaceutically acceptable salt thereof.

64. The method of claim 62 or 63, further comprising administering a combination agent to the patient.

65. The method of claim 64, wherein the combination agent is carboplatin.

66. The method of claim 64, wherein the combination agent is paclitaxel.

67. The method of any one of claims 62-66, further comprising administering pemetrexed to the patient.

68. The method of any one of claims 62-67, wherein the patient has previously been administered at least one prior line of therapy.

69. The method of claim 68, wherein the therapy is a platinum-based therapy.

70. The method of any one of claims 62-69, wherein the patient previously been administered carboplatin and/or pemetrexed.

71. The method any one of claims 62-70, wherein the patient previously been administered cisplatin and/or pemetrexed.

72. The method of any one of claims 62-71, comprising administering to the patient about 50 mg to about 200 mg of the compound of Formula (I) or pharmaceutically acceptable salt thereof per day.

73. The method of any one of claims 62-71, comprising administering to the patient about 50 mg of the compound of Formula (I) or pharmaceutically acceptable salt thereof twice per day.

74. The method of any one of claims 62-71, comprising administering to the patient about 100 mg of the compound of Formula (I) or pharmaceutically acceptable salt thereof twice per day.

75. The method of any one of claims 62-71, wherein the compound of Formula (I) or pharmaceutically acceptable salt thereof is orally administered to the patient.

76. The method of any one of claims 64-71, wherein the combination agent is intravenously administered to the patient.

77. The method of any one of claims 62-76, wherein the combination agent is intravenously administered to the patient in an amount sufficient to provide an area under the curve of about 5 to about 7.

78. The method of any one of claims 62-77, comprising reducing each of the amount of the compound of Formula (I) or pharmaceutically acceptable salt thereof and the combination agent administered to the patient after one or more months of administering to the patient.

79. The method of any one of claims 62-78, comprising reducing each of the amount of the compound of Formula (I) or pharmaceutically acceptable salt thereof and the combination agent administered to the patient after one or more months of administering to the patient.

80. A method of treating ovarian cancer in a patient in need thereof, comprising administering to the patient 50 mg, twice per day, of a compound of Formula (I): 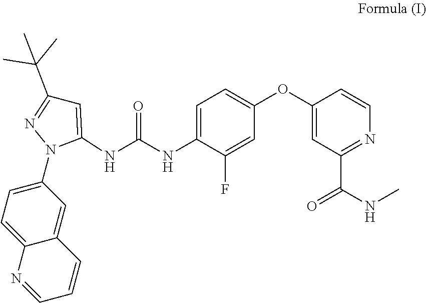 [SMILES: CNC(=O)c1cc(Oc2ccc(NC(=O)Nc3cc(C(C)(C)C)nn3-c3ccc4ncccc4c3)c(F)c2)ccn1]
and 80 mg/m² of paclitaxel once per week.

81. The method of claim 80, wherein the patient had received at least one prior platinum therapy.

82. The method of claim 80 or 81, wherein the ovarian cancer is a platinum-resistant ovarian cancer.

83. A method of treating ovarian cancer in a patient in need thereof, comprising administering to the patient 100 mg, twice per day, of a compound of Formula (I): 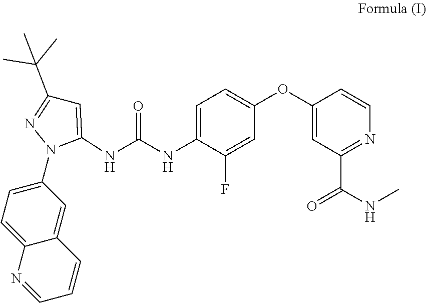 [SMILES: CNC(=O)c1cc(Oc2ccc(NC(=O)Nc3cc(C(C)(C)C)nn3-c3ccc4ncccc4c3)c(F)c2)ccn1]
and 80 mg/m² of paclitaxel once per week.

84. The method of claim 83, wherein the patient had received at least one prior platinum therapy.

85. The method of claim 83 or 84, wherein the ovarian cancer is a platinum-resistant ovarian cancer.

86. A method of treating pancreatic ductal adenocarcinoma in a patient in need thereof, comprising administering to the patient an effective amount of a compound of Formula (I) 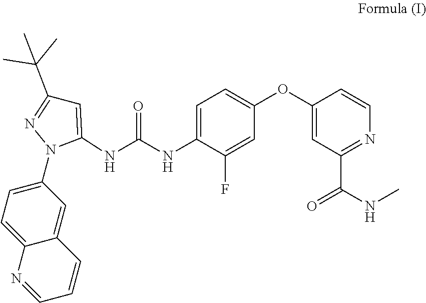 [SMILES: CNC(=O)c1cc(Oc2ccc(NC(=O)Nc3cc(C(C)(C)C)nn3-c3ccc4ncccc4c3)c(F)c2)ccn1]
or a pharmaceutically acceptable salt thereof.

87. A method for achieving at least a partial response in a patient having pancreatic ductal adenocarcinoma, comprising administering to the patient an effective amount of the compound of Formula (I) 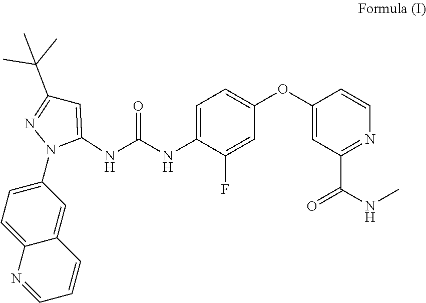 [SMILES: CNC(=O)c1cc(Oc2ccc(NC(=O)Nc3cc(C(C)(C)C)nn3-c3ccc4ncccc4c3)c(F)c2)ccn1]
or a pharmaceutically acceptable salt thereof.

88. A method for achieving a stable disease condition in a patient having pancreatic ductal adenocarcinoma, comprising administering to the patient an effective amount of the compound of Formula (I) 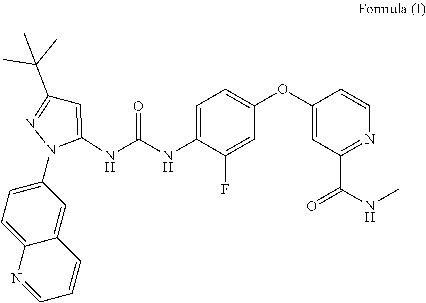 [SMILES: CNC(=O)c1cc(Oc2ccc(NC(=O)Nc3cc(C(C)(C)C)nn3-c3ccc4ncccc4c3)c(F)c2)ccn1]
or a pharmaceutically acceptable salt thereof.

89. The method of any one of claims 86-88, wherein the patient has previously been administered at least one prior line of platinum-based therapy.

90. The method of any one of claims 86-89, wherein after 28 days or more of administering the compound of Formula (I) or pharmaceutically acceptable salt thereof to the patient, the patient has at least a partial response or a stable disease condition.

91. The method of any one of claims 86-90, comprising administering to the patient about 100 mg of Formula (I) or pharmaceutically acceptable salt thereof per day.

92. The method of any one of claims 86-90, comprising administering to the patient about 150 mg of the compound of Formula (I) or pharmaceutically acceptable salt thereof per day.

93. The method of any one of claims 86-90, comprising administering to the patient about 200 mg of the compound of Formula (I) or pharmaceutically acceptable salt thereof per day.

94. The method of any one of claims 86-90, comprising administering to the patient about 50 mg of the compound of Formula (I) or pharmaceutically acceptable salt thereof twice per day.

95. The method of any one of claims 86-90, comprising administering to the patient about 100 mg of the compound of Formula (I) or pharmaceutically acceptable salt thereof twice per day.

96. The method of any one of claims 86-95, further comprising administering a combination agent to the patient.

97. The method of any one of claims 86-96, wherein the compound of Formula (I) or pharmaceutically acceptable salt thereof is orally administered to the patient.

98. The method of claim 96, wherein the combination agent is carboplatin.

99. The method of claim 96, wherein the combination agent is paclitaxel.

100. The method of any one of claims 86-99, further comprising administering pemetrexed to the patient.

101. The method of any one of claims 86-100, wherein the patient has previously been administered at least one prior line of therapy.

102. The method of any one of claim 101, wherein the at least one prior line of therapy is a platinum-based therapy.

103. The method of any one of claims 86-102, wherein the patient has previously been administered carboplatin and/or pemetrexed.

104. The method of any one of claims 86-102, wherein the patient has previously been administered cisplatin and/or pemetrexed.

105. The method of any one of claims 96-104, wherein the combination agent is intravenously administered to the patient.

106. A method of treating lung cancer in a patient in need thereof, comprising administering to the patient an effective amount of a compound of Formula (I) 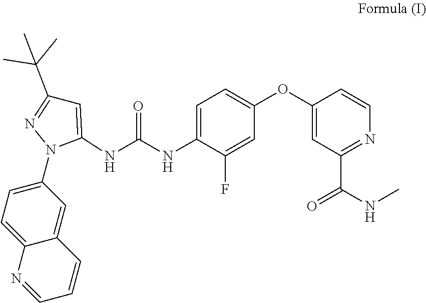 [SMILES: CNC(=O)c1cc(Oc2ccc(NC(=O)Nc3cc(C(C)(C)C)nn3-c3ccc4ncccc4c3)c(F)c2)ccn1]
or a pharmaceutically acceptable salt thereof.

107. A method for achieving at least a partial response in a patient having lung cancer, comprising administering to the patient an effective amount of the compound of Formula (I) 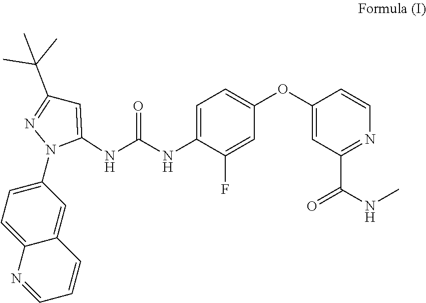 [SMILES: CNC(=O)c1cc(Oc2ccc(NC(=O)Nc3cc(C(C)(C)C)nn3-c3ccc4ncccc4c3)c(F)c2)ccn1]
or a pharmaceutically acceptable salt thereof.

108. A method for achieving a stable disease condition in a patient having lung cancer, comprising administering to the patient an effective amount of the compound of Formula (I) 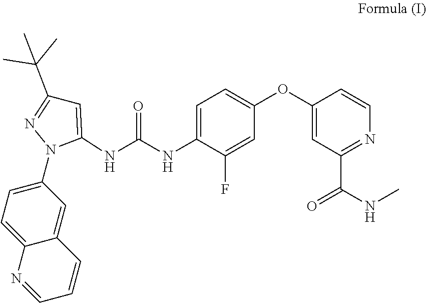 [SMILES: CNC(=O)c1cc(Oc2ccc(NC(=O)Nc3cc(C(C)(C)C)nn3-c3ccc4ncccc4c3)c(F)c2)ccn1]
or a pharmaceutically acceptable salt thereof.

109. The method of any one of claims 106-108, wherein the patient has previously been administered at least one prior line of platinum-based therapy.

110. The method of any one of claims 106-109, wherein after 28 days or more of administering the compound of Formula (I) or pharmaceutically acceptable salt thereof to the patient, the patient has at least a partial response or a stable disease condition.

111. The method of any one of claims 106-110, comprising administering to the patient about 100 mg of Formula (I) or pharmaceutically acceptable salt thereof per day.

112. The method of any one of claims 106-110, comprising administering to the patient about 150 mg of the compound of Formula (I) or pharmaceutically acceptable salt thereof per day.

113. The method of any one of claims 106-110, comprising administering to the patient about 200 mg of the compound of Formula (I) or pharmaceutically acceptable salt thereof per day.

114. The method of any one of claims 106-110, comprising administering to the patient about 50 mg of the compound of Formula (I) or pharmaceutically acceptable salt thereof twice per day.

115. The method of any one of claims 106-110, comprising administering to the patient about 100 mg of the compound of Formula (I) or pharmaceutically acceptable salt thereof twice per day.

116. The method of any one of claims 106-115, further comprising administering a combination agent to the patient.

117. The method of any one of claims 106-116, wherein the compound of Formula (I) or pharmaceutically acceptable salt thereof is orally administered to the patient.

118. The method of claim 116, wherein the combination agent is carboplatin.

119. The method of claim 116, wherein the combination agent is paclitaxel.

120. The method of any one of claims 106-119, further comprising administering pemetrexed to the patient.

121. The method of any one of claims 106-120, wherein the patient has previously been administered at least one prior line of therapy.

122. The method of any one of claim 121, wherein the at least one prior line of therapy is a platinum-based therapy.

123. The method of any one of claims 106-122, wherein the patient has previously been administered carboplatin and/or pemetrexed.

124. The method of any one of claims 106-122, wherein the patient has previously been administered cisplatin and/or pemetrexed.

125. The method of any one of claims 116-124, wherein the combination agent is intravenously administered to the patient.
